SEQUENCE LISTING (558 sequences)

SEQ ID NO: 1 - protein, 27 aa, synthetic construct
DSWQEEVIKLCGRELVRAQIAICGKST

SEQ ID NO: 2 - protein, 27 aa, synthetic construct
DSWQEEVIKLCGRELVRAQIAICGQST

SEQ ID NO: 3 - protein, 27 aa, synthetic construct
DSYQEEVIKLCGRELVRAQIAICGKST

SEQ ID NO: 4 - protein, 27 aa, synthetic construct
DSFQEEVIKLCGRELVRAQIAICGKST

SEQ ID NO: 5 - protein, 27 aa, synthetic construct
DSLQEEVIKLCGRELVRAQIAICGKST

SEQ ID NO: 6 - protein, 27 aa, synthetic construct
DSIQEEVIKLCGRELVRAQIAICGKST

SEQ ID NO: 7 - protein, 27 aa, synthetic construct
  VARIANT 3: note = any amino acid except for M, H, and C
  VARIANT 9: note = K, Q, D, E, L, I or Y
  VARIANT 25: note = M, K, Q, or N
DSXQEEVIXLCGRELVRAQIAICGXST

SEQ ID NO: 8 - protein, 24 aa, synthetic construct
QLYSALANKCCHVGCTKRSLAQFC

SEQ ID NO: 9 - protein, 24 aa, synthetic construct
QLYSALANKCCHVGCTKQSLAQFC

SEQ ID NO: 10 - protein, 24 aa, synthetic construct
QLYSALANKCCYVGCTKRSLAQFC

SEQ ID NO: 11 - protein, 24 aa, synthetic construct
QLYSALANKCCLVGCTKRSLAQFC

SEQ ID NO: 12 - protein, 24 aa, synthetic construct
QLYSALANKCCQVGCTKRSLAQFC

SEQ ID NO: 13 - protein, 24 aa, synthetic construct
QLYSALANKCCKVGCTKRSLAQFC

SEQ ID NO: 14 - protein, 24 aa, synthetic construct
QLYSALANKCCYVGCTKQSLAQFC

SEQ ID NO: 15 - protein, 24 aa, synthetic construct
QLYSALANKCCKVGCTKQSLAQFC

SEQ ID NO: 16 - protein, 24 aa, synthetic construct
  VARIANT 9: note = K, Q, D, E, L, I, or Y
  VARIANT 12: note = any amino acid except for M, W, and C
  VARIANT 17: note = K, Q, D, E, L, I, or Y
  VARIANT 18: note = Q, D, E, L, I, Y or R
QLYSALANXCCXVGCTXXSLAQFC

SEQ ID NO: 17 - protein, 13 aa, synthetic construct
  VARIANT 8: note = D, E, N, or Q
  VARIANT 10: note = D, E, N, or Q
ASDAAGAXAXAGA

SEQ ID NO: 18 - protein, 13 aa, synthetic construct
ASDAAGADADAGA

SEQ ID NO: 19 - protein, 13 aa, synthetic construct
ASDAAGADANAGA

SEQ ID NO: 20 - protein, 13 aa, synthetic construct
ASDAAGADAQAGA

SEQ ID NO: 21 - protein, 13 aa, synthetic construct
ASDAAGAEAEAGA

SEQ ID NO: 22 - protein, 13 aa, synthetic construct
ASDAAGANADAGA

SEQ ID NO: 23 - protein, 13 aa, synthetic construct
ASDAAGAQADAGA

SEQ ID NO: 24 - protein, 13 aa, synthetic construct
ASDAAGAQAQAGA

SEQ ID NO: 25 - protein, 13 aa, synthetic construct
  VARIANT 10: note = E or S
GGEGSGGEGXGGG

SEQ ID NO: 26 - protein, 13 aa, synthetic construct
GGEGSGGEGEGGG

SEQ ID NO: 27 - protein, 13 aa, synthetic construct
GGEGSGGEGSGGG

SEQ ID NO: 28 - protein, 65 aa, synthetic construct
DSWQEEVIKLCGRELVRAQIAICGKSTASDAAGADANAGARQLYSALANKCCHVGCTKRSLAQFC

SEQ ID NO: 29 - protein, 65 aa, synthetic construct
DSWQEEVIKLCGRELVRAQIAICGKSTASDAAGANADAGARQLYSALANKCCHVGCTKRSLAQFC

SEQ ID NO: 30 - protein, 65 aa, synthetic construct
DSWQEEVIKLCGRELVRAQIAICGKSTASDAAGADADAGARQLYSALANKCCHVGCTKRSLAQFC

SEQ ID NO: 31 - protein, 65 aa, synthetic construct
DSWQEEVIKLCGRELVRAQIAICGKSTASDAAGAEAEAGARQLYSALANKCCHVGCTKRSLAQFC

SEQ ID NO: 32 - protein, 65 aa, synthetic construct
DSWQEEVIKLCGRELVRAQIAICGKSTASDAAGAQADAGARQLYSALANKCCHVGCTKRSLAQFC

SEQ ID NO: 33 - protein, 65 aa, synthetic construct
DSWQEEVIKLCGRELVRAQIAICGKSTASDAAGADAQAGARQLYSALANKCCHVGCTKRSLAQFC

SEQ ID NO: 34 - protein, 65 aa, synthetic construct
DSWQEEVIKLCGRELVRAQIAICGKSTASDAAGAQAQAGARQLYSALANKCCHVGCTKRSLAQFC

SEQ ID NO: 35 - protein, 65 aa, synthetic construct
DSWQEEVIKLCGRELVRAQIAICGQSTASDAAGADANAGARQLYSALANKCCHVGCTKRSLAQFC

SEQ ID NO: 36 - protein, 65 aa, synthetic construct
DSWQEEVIKLCGRELVRAQIAICGQSTASDAAGANADAGARQLYSALANKCCHVGCTKRSLAQFC

SEQ ID NO: 37 - protein, 65 aa, synthetic construct
DSWQEEVIKLCGRELVRAQIAICGQSTASDAAGADADAGARQLYSALANKCCHVGCTKRSLAQFC

SEQ ID NO: 38 - protein, 65 aa, synthetic construct
DSWQEEVIKLCGRELVRAQIAICGQSTASDAAGAEAEAGARQLYSALANKCCHVGCTKRSLAQFC

SEQ ID NO: 39 - protein, 65 aa, synthetic construct
DSWQEEVIKLCGRELVRAQIAICGQSTASDAAGAQADAGARQLYSALANKCCHVGCTKRSLAQFC

SEQ ID NO: 40 - protein, 65 aa, synthetic construct
DSWQEEVIKLCGRELVRAQIAICGQSTASDAAGADAQAGARQLYSALANKCCHVGCTKRSLAQFC

SEQ ID NO: 41 - protein, 65 aa, synthetic construct
DSWQEEVIKLCGRELVRAQIAICGQSTASDAAGAQAQAGARQLYSALANKCCHVGCTKRSLAQFC

SEQ ID NO: 42 - protein, 65 aa, synthetic construct
DSWQEEVIKLCGRELVRAQIAICGKSTASDAAGADANAGARQLYSALANKCCHVGCTKQSLAQFC

SEQ ID NO: 43 - protein, 65 aa, synthetic construct
DSWQEEVIKLCGRELVRAQIAICGKSTASDAAGANADAGARQLYSALANKCCHVGCTKQSLAQFC

SEQ ID NO: 44 - protein, 65 aa, synthetic construct
DSWQEEVIKLCGRELVRAQIAICGKSTASDAAGADADAGARQLYSALANKCCHVGCTKQSLAQFC

SEQ ID NO: 45 - protein, 65 aa, synthetic construct
DSWQEEVIKLCGRELVRAQIAICGKSTASDAAGAEAEAGARQLYSALANKCCHVGCTKQSLAQFC

SEQ ID NO: 46 - protein, 65 aa, synthetic construct
DSWQEEVIKLCGRELVRAQIAICGKSTASDAAGAQADAGARQLYSALANKCCHVGCTKQSLAQFC

SEQ ID NO: 47 - protein, 65 aa, synthetic construct
DSWQEEVIKLCGRELVRAQIAICGKSTASDAAGADAQAGARQLYSALANKCCHVGCTKQSLAQFC

SEQ ID NO: 48 - protein, 65 aa, synthetic construct
DSWQEEVIKLCGRELVRAQIAICGKSTASDAAGAQAQAGARQLYSALANKCCHVGCTKQSLAQFC

SEQ ID NO: 49 - protein, 65 aa, synthetic construct
DSWQEEVIKLCGRELVRAQIAICGQSTASDAAGADANAGARQLYSALANKCCHVGCTKQSLAQFC

SEQ ID NO: 50 - protein, 65 aa, synthetic construct
DSWQEEVIKLCGRELVRAQIAICGQSTASDAAGANADAGARQLYSALANKCCHVGCTKQSLAQFC

SEQ ID NO: 51 - protein, 65 aa, synthetic construct
DSWQEEVIKLCGRELVRAQIAICGQSTASDAAGADADAGARQLYSALANKCCHVGCTKQSLAQFC

SEQ ID NO: 52 - protein, 65 aa, synthetic construct
DSWQEEVIKLCGRELVRAQIAICGQSTASDAAGAEAEAGARQLYSALANKCCHVGCTKQSLAQFC

SEQ ID NO: 53 - protein, 65 aa, synthetic construct
DSWQEEVIKLCGRELVRAQIAICGQSTASDAAGAQADAGARQLYSALANKCCHVGCTKQSLAQFC

SEQ ID NO: 54 - protein, 65 aa, synthetic construct
DSWQEEVIKLCGRELVRAQIAICGQSTASDAAGADAQAGARQLYSALANKCCHVGCTKQSLAQFC

SEQ ID NO: 55 - protein, 65 aa, synthetic construct
DSWQEEVIKLCGRELVRAQIAICGQSTASDAAGAQAQAGARQLYSALANKCCHVGCTKQSLAQFC

SEQ ID NO: 56 - protein, 65 aa, synthetic construct
DSYQEEVIKLCGRELVRAQIAICGKSTASDAAGAEAEAGARQLYSALANKCCHVGCTKRSLAQFC

SEQ ID NO: 57 - protein, 65 aa, synthetic construct
DSFQEEVIKLCGRELVRAQIAICGKSTASDAAGAEAEAGARQLYSALANKCCHVGCTKRSLAQFC

SEQ ID NO: 58 - protein, 65 aa, synthetic construct
DSLQEEVIKLCGRELVRAQIAICGKSTASDAAGAEAEAGARQLYSALANKCCHVGCTKRSLAQFC

SEQ ID NO: 59 - protein, 65 aa, synthetic construct
DSIQEEVIKLCGRELVRAQIAICGKSTASDAAGAEAEAGARQLYSALANKCCHVGCTKRSLAQFC

SEQ ID NO: 60 - protein, 65 aa, synthetic construct
DSWQEEVIKLCGRELVRAQIAICGKSTASDAAGAEAEAGARQLYSALANKCCYVGCTKRSLAQFC

SEQ ID NO: 61 - protein, 65 aa, synthetic construct
DSWQEEVIKLCGRELVRAQIAICGKSTASDAAGAEAEAGARQLYSALANKCCLVGCTKRSLAQFC

SEQ ID NO: 62 - protein, 65 aa, synthetic construct
DSWQEEVIKLCGRELVRAQIAICGKSTASDAAGAEAEAGARQLYSALANKCCQVGCTKRSLAQFC

SEQ ID NO: 63 - protein, 65 aa, synthetic construct
DSWQEEVIKLCGRELVRAQIAICGKSTASDAAGAEAEAGARQLYSALANKCCKVGCTKRSLAQFC

SEQ ID NO: 64 - protein, 65 aa, synthetic construct
DSYQEEVIKLCGRELVRAQIAICGKSTASDAAGAEAEAGARQLYSALANKCCYVGCTKRSLAQFC

SEQ ID NO: 65 - protein, 65 aa, synthetic construct
DSYQEEVIKLCGRELVRAQIAICGKSTASDAAGAEAEAGARQLYSALANKCCKVGCTKRSLAQFC

SEQ ID NO: 66 - protein, 65 aa, synthetic construct
DSYQEEVIKLCGRELVRAQIAICGKSTGGEGSGGEGEGGGRQLYSALANKCCYVGCTKRSLAQFC

SEQ ID NO: 67 - protein, 65 aa, synthetic construct
DSYQEEVIKLCGRELVRAQIAICGKSTGGEGSGGEGEGGGRQLYSALANKCCKVGCTKRSLAQFC

SEQ ID NO: 68 - protein, 65 aa, synthetic construct
DSYQEEVIKLCGRELVRAQIAICGKSTGGEGSGGEGSGGGRQLYSALANKCCYVGCTKRSLAQFC

SEQ ID NO: 69 - protein, 65 aa, synthetic construct
DSYQEEVIKLCGRELVRAQIAICGKSTGGEGSGGEGSGGGRQLYSALANKCCKVGCTKRSLAQFC

SEQ ID NO: 70 - protein, 65 aa, synthetic construct
DSYQEEVIKLCGRELVRAQIAICGKSTASDAAGAEAEAGARQLYSALANKCCYVGCTKQSLAQFC

SEQ ID NO: 71 - protein, 65 aa, synthetic construct
DSYQEEVIKLCGRELVRAQIAICGKSTASDAAGAEAEAGARQLYSALANKCCKVGCTKQSLAQFC

SEQ ID NO: 72 - protein, 65 aa, synthetic construct
DSYQEEVIKLCGRELVRAQIAICGKSTGGEGSGGEGEGGGRQLYSALANKCCYVGCTKQSLAQFC

SEQ ID NO: 73 - protein, 65 aa, synthetic construct
DSYQEEVIKLCGRELVRAQIAICGKSTGGEGSGGEGEGGGRQLYSALANKCCKVGCTKQSLAQFC

SEQ ID NO: 74 - protein, 65 aa, synthetic construct
DSYQEEVIKLCGRELVRAQIAICGKSTGGEGSGGEGSGGGRQLYSALANKCCYVGCTKQSLAQFC

SEQ ID NO: 75 - protein, 65 aa, synthetic construct
DSYQEEVIKLCGRELVRAQIAICGKSTGGEGSGGEGSGGGRQLYSALANKCCKVGCTKQSLAQFC

SEQ ID NO: 76 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 80 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 84 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGADANAGARQLYSALANKCCHVGCTKRSLAQFC

SEQ ID NO: 85 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGANADAGARQLYSALANKCCHVGCTKRSLAQFC

SEQ ID NO: 86 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGADADAGARQLYSALANKCCHVGCTKRSLAQFC

SEQ ID NO: 87 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAEAEAGARQLYSALANKCCHVGCTKRSLAQFC

SEQ ID NO: 88 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAQADAGARQLYSALANKCCHVGCTKRSLAQFC

SEQ ID NO: 89 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGADAQAGARQLYSALANKCCHVGCTKRSLAQFC

SEQ ID NO: 90 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAQAQAGARQLYSALANKCCHVGCTKRSLAQFC

SEQ ID NO: 91 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADANAGARQLYSALANKCCHVGCTKRSLAQFC

SEQ ID NO: 92 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGANADAGARQLYSALANKCCHVGCTKRSLAQFC

SEQ ID NO: 93 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADADAGARQLYSALANKCCHVGCTKRSLAQFC

SEQ ID NO: 94 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAEAEAGARQLYSALANKCCHVGCTKRSLAQFC

SEQ ID NO: 95 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAQADAGARQLYSALANKCCHVGCTKRSLAQFC

SEQ ID NO: 96 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADAQAGARQLYSALANKCCHVGCTKRSLAQFC

SEQ ID NO: 97 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAQAQAGARQLYSALANKCCHVGCTKRSLAQFC

SEQ ID NO: 98 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADANAGARQLYSALANKCCHVGCTKRSLAQFC

SEQ ID NO: 99 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGANADAGARQLYSALANKCCHVGCTKRSLAQFC

SEQ ID NO: 100 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADADAGARQLYSALANKCCHVGCTKRSLAQFC

SEQ ID NO: 101 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAEAEAGARQLYSALANKCCHVGCTKRSLAQFC

SEQ ID NO: 102 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAQADAGARQLYSALANKCCHVGCTKRSLAQFC

SEQ ID NO: 103 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADAQAGARQLYSALANKCCHVGCTKRSLAQFC

SEQ ID NO: 104 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAQAQAGARQLYSALANKCCHVGCTKRSLAQFC

SEQ ID NO: 105 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADANAGARQLYSALANKCCHVGCTKQSLAQFC

SEQ ID NO: 106 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGANADAGARQLYSALANKCCHVGCTKQSLAQFC

SEQ ID NO: 107 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADADAGARQLYSALANKCCHVGCTKQSLAQFC

SEQ ID NO: 108 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAEAEAGARQLYSALANKCCHVGCTKQSLAQFC

SEQ ID NO: 109 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAQADAGARQLYSALANKCCHVGCTKQSLAQFC

SEQ ID NO: 110 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADAQAGARQLYSALANKCCHVGCTKQSLAQFC

SEQ ID NO: 111 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAQAQAGARQLYSALANKCCHVGCTKQSLAQFC

SEQ ID NO: 112 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADANAGARQLYSALANKCCHVGCTKQSLAQFC

SEQ ID NO: 113 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGANADAGARQLYSALANKCCHVGCTKQSLAQFC

SEQ ID NO: 114 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADADAGARQLYSALANKCCHVGCTKQSLAQFC

SEQ ID NO: 115 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAEAEAGARQLYSALANKCCHVGCTKQSLAQFC

SEQ ID NO: 116 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAQADAGARQLYSALANKCCHVGCTKQSLAQFC

SEQ ID NO: 117 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADAQAGARQLYSALANKCCHVGCTKQSLAQFC

SEQ ID NO: 118 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAQAQAGARQLYSALANKCCHVGCTKQSLAQFC

SEQ ID NO: 119 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTASDAAGAEAEAGARQLYSALANKCCHVGCTKRSLAQFC

SEQ ID NO: 120 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSFQEEVIKLCGRELVRAQIAICGKSTASDAAGAEAEAGARQLYSALANKCCHVGCTKRSLAQFC

SEQ ID NO: 121 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSLQEEVIKLCGRELVRAQIAICGKSTASDAAGAEAEAGARQLYSALANKCCHVGCTKRSLAQFC

SEQ ID NO: 122 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSIQEEVIKLCGRELVRAQIAICGKSTASDAAGAEAEAGARQLYSALANKCCHVGCTKRSLAQFC

SEQ ID NO: 123 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAEAEAGARQLYSALANKCCYVGCTKRSLAQFC

SEQ ID NO: 124 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAEAEAGARQLYSALANKCCLVGCTKRSLAQFC

SEQ ID NO: 125 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAEAEAGARQLYSALANKCCQVGCTKRSLAQFC

SEQ ID NO: 126 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAEAEAGARQLYSALANKCCKVGCTKRSLAQFC

SEQ ID NO: 127 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTASDAAGAEAEAGARQLYSALANKCCYVGCTKRSLAQFC

SEQ ID NO: 128 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTASDAAGAEAEAGARQLYSALANKCCKVGCTKRSLAQFC

SEQ ID NO: 129 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTGGEGSGGEGEGGGRQLYSALANKCCYVGCTKRSLAQFC

SEQ ID NO: 130 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTGGEGSGGEGEGGGRQLYSALANKCCKVGCTKRSLAQFC

SEQ ID NO: 131 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTGGEGSGGEGSGGGRQLYSALANKCCYVGCTKRSLAQFC

SEQ ID NO: 132 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTGGEGSGGEGSGGGRQLYSALANKCCKVGCTKRSLAQFC

SEQ ID NO: 133 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTASDAAGAEAEAGARQLYSALANKCCYVGCTKQSLAQFC

SEQ ID NO: 134 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTASDAAGAEAEAGARQLYSALANKCCKVGCTKQSLAQFC

SEQ ID NO: 135 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCYVGCTKQSLAQFC

SEQ ID NO: 136 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCKVGCTKQSLAQFC

SEQ ID NO: 137 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCYVGCTKQSLAQFC

SEQ ID NO: 138 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCKVGCTKQSLAQFC

SEQ ID NO: 139 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGADANAGARQLYSALANKCCHVGCTKRSLAQFC

SEQ ID NO: 140 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGANADAGARQLYSALANKCCHVGCTKRSLAQFC

SEQ ID NO: 141 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGADADAGARQLYSALANKCCHVGCTKRSLAQFC

SEQ ID NO: 142 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAEAEAGARQLYSALANKCCHVGCTKRSLAQFC

SEQ ID NO: 143 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAQADAGARQLYSALANKCCHVGCTKRSLAQFC

SEQ ID NO: 144 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGADAQAGARQLYSALANKCCHVGCTKRSLAQFC

SEQ ID NO: 145 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAQAQAGARQLYSALANKCCHVGCTKRSLAQFC

SEQ ID NO: 146 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADANAGARQLYSALANKCCHVGCTKRSLAQFC

SEQ ID NO: 147 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGANADAGARQLYSALANKCCHVGCTKRSLAQFC

SEQ ID NO: 148 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADADAGARQLYSALANKCCHVGCTKRSLAQFC

SEQ ID NO: 149 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAEAEAGARQLYSALANKCCHVGCTKRSLAQFC

SEQ ID NO: 150 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAQADAGARQLYSALANKCCHVGCTKRSLAQFC

SEQ ID NO: 151 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADAQAGARQLYSALANKCCHVGCTKRSLAQFC

SEQ ID NO: 152 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAQAQAGARQLYSALANKCCHVGCTKRSLAQFC

SEQ ID NO: 153 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADANAGARQLYSALANKCCHVGCTKRSLAQFC

SEQ ID NO: 154 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGANADAGARQLYSALANKCCHVGCTKRSLAQFC

SEQ ID NO: 155 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADADAGARQLYSALANKCCHVGCTKRSLAQFC

SEQ ID NO: 156 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAEAEAGARQLYSALANKCCHVGCTKRSLAQFC

SEQ ID NO: 157 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAQADAGARQLYSALANKCCHVGCTKRSLAQFC

SEQ ID NO: 158 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADAQAGARQLYSALANKCCHVGCTKRSLAQFC

SEQ ID NO: 159 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAQAQAGARQLYSALANKCCHVGCTKRSLAQFC

SEQ ID NO: 160 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADANAGARQLYSALANKCCHVGCTKQSLAQFC

SEQ ID NO: 161 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGANADAGARQLYSALANKCCHVGCTKQSLAQFC

SEQ ID NO: 162 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADADAGARQLYSALANKCCHVGCTKQSLAQFC

SEQ ID NO: 163 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAEAEAGARQLYSALANKCCHVGCTKQSLAQFC

SEQ ID NO: 164 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAQADAGARQLYSALANKCCHVGCTKQSLAQFC

SEQ ID NO: 165 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGADAQAGARQLYSALANKCCHVGCTKQSLAQFC

SEQ ID NO: 166 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGKSTASDAAGAQAQAGARQLYSALANKCCHVGCTKQSLAQFC

SEQ ID NO: 167 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADANAGARQLYSALANKCCHVGCTKQSLAQFC

SEQ ID NO: 168 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGANADAGARQLYSALANKCCHVGCTKQSLAQFC

SEQ ID NO: 169 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADADAGARQLYSALANKCCHVGCTKQSLAQFC

SEQ ID NO: 170 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAEAEAGARQLYSALANKCCHVGCTKQSLAQFC

SEQ ID NO: 171 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAQADAGARQLYSALANKCCHVGCTKQSLAQFC

SEQ ID NO: 172 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGADAQAGARQLYSALANKCCHVGCTKQSLAQFC

SEQ ID NO: 173 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSWQEEVIKLCGRELVRAQIAICGQSTASDAAGAQAQAGARQLYSALANKCCHVGCTKQSLAQFC

SEQ ID NO: 174 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTASDAAGAEAEAGARQLYSALANKCCHVGCTKRSLAQFC

SEQ ID NO: 175 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSFQEEVIKLCGRELVRAQIAICGKSTASDAAGAEAEAGARQLYSALANKCCHVGCTKRSLAQFC

SEQ ID NO: 176 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSLQEEVIKLCGRELVRAQIAICGKSTASDAAGAEAEAGARQLYSALANKCCHVGCTKRSLAQFC

SEQ ID NO: 177 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSIQEEVIKLCGRELVRAQIAICGKSTASDAAGAEAEAGARQLYSALANKCCHVGCTKRSLAQFC

SEQ ID NO: 178 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAEAEAGARQLYSALANKCCYVGCTKRSLAQFC

SEQ ID NO: 179 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAEAEAGARQLYSALANKCCLVGCTKRSLAQFC

SEQ ID NO: 180 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAEAEAGARQLYSALANKCCQVGCTKRSLAQFC

SEQ ID NO: 181 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAEAEAGARQLYSALANKCCKVGCTKRSLAQFC

SEQ ID NO: 182 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTASDAAGAEAEAGARQLYSALANKCCYVGCTKRSLAQFC

SEQ ID NO: 183 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTASDAAGAEAEAGARQLYSALANKCCKVGCTKRSLAQFC

SEQ ID NO: 184 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTGGEGSGGEGEGGGRQLYSALANKCCYVGCTKRSLAQFC

SEQ ID NO: 185 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTGGEGSGGEGEGGGRQLYSALANKCCKVGCTKRSLAQFC

SEQ ID NO: 186 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTGGEGSGGEGSGGGRQLYSALANKCCYVGCTKRSLAQFC

SEQ ID NO: 187 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGEGGSDSYQEEVIKLCGRELVRAQIAICGKSTGGEGSGGEGSGGGRQLYSALANKCCKVGCTKRSLAQFC

SEQ ID NO: 188 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTASDAAGAEAEAGARQLYSALANKCCYVGCTKQSLAQFC

SEQ ID NO: 189 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTASDAAGAEAEAGARQLYSALANKCCKVGCTKQSLAQFC

SEQ ID NO: 190 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCYVGCTKQSLAQFC

SEQ ID NO: 191 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCKVGCTKQSLAQFC

SEQ ID NO: 192 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCYVGCTKQSLAQFC

SEQ ID NO: 193 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCKVGCTKQSLAQFC

SEQ ID NO: 194 - DNA, 945 bp, synthetic construct
  source 1..945: mol_type = other DNA
atggagactgacacactcctcctttgggttttgctgttgtgggtgcctggatctactggcgacaagacccatacatgtccgccttgtcctgcgcctgaggcagcaggcggaccatcagtcttcttgtttccccccaagccgaaggacacccttatgatctcacgcacccccgaagtaacttgtgtagtcgttgatgtctcacacgaagacccggaagtaaagtttaattggtatgtcgatggtgttgaggtccacaacgctaaaacgaaaccgcgggaagaacaatacaactccacatatcgagtagtctccgtcctgactgttcttcaccaggactggctgaatggtaaagaatacaaatgtaaagtgagtaacaaggcccttgcagcacccatcgagaagacgatatccaaagccaaggggcaaccgcgcgagccacaagtttacacgctcccaccctcaagagacgaactcaccaaaaatcaagtgtccctgacatgtctggtgaaaggattctatcccagcgacatagctgtagaatgggagagtaatggccaacccgaaaacaattacaaaactacccccccggttttggatagtgatggttcattcttcctctatagtaaacttaccgtggataagtctcggtggcagcaggggaacgtgtttagctgttcagtcctccatgaggcactccatagtcactatacgcaaaagtcattgtccctttctccgggcaagggcgggtcagactcctggcaggaagaggtaattaagctttgtgggcgagaactcgttagggcacagatagcaatctgcgggaaaagtacagcttccgatgctgccggggctgacgccaatgcgggagcacgccagctctactcagccctcgccaacaagtgttgtcatgtaggttgcaccaaaagaagtctggcacagttttgc

SEQ ID NO: 195 - DNA, 945 bp, synthetic construct
  source 1..945: mol_type = other DNA
atggaaacagacactcttcttttgtgggtcttgttgctctgggtccctggctcaacaggcgacaagacgcatacttgtcctccctgcccagctcccgaagcggctggggggccctctgtctttctgtttccgcctaagcccaaggacacgctcatgataagtcgcactccggaagtcacctgtgttgtcgtcgatgttagccatgaagatccagaggtgaaatttaactggtacgtcgacggagtggaggttcacaatgctaaaaccaaaccgcgagaagagcaatacaattccacgtatagggtcgtctccgtcctgacagtactccatcaggattggctgaatggaaaagaatacaagtgcaaggtttccaataaagccttggccgcacctattgagaaaacgatatcaaaagctaagggacaacctcgggagccgcaagtatatacactccccccttctagggacgaactgacaaaaaaccaagttagtctgacttgtcttgtgaaaggtttttacccgagtgatatagccgtagaatgggagagcaatggccagcccgaaaacaattacaaaacaactcccccagtattggacagtgacgggtcattttttttgtattctaaattgaccgtagacaagtcacgctggcaacaagggaatgtatttagctgttccgtccttcatgaggcgctccatagccattacactcagaagtctttgtcactgtcaccgggcaagggtggttctgattcatggcaagaggaagtgattaagctgtgcggtcgggagttggtaagagctcaaattgcgatttgtggcaagagcactgcgtccgatgccgcaggtgctaatgccgacgccggtgcgagacagctttattctgcgctggccaacaagtgctgccacgtcggatgcaccaaacggagccttgctcagttttgc

SEQ ID NO: 196 - DNA, 945 bp, synthetic construct
  source 1..945: mol_type = other DNA
atggaaaccgacaccctgcttctctgggtgctgctgctctgggtaccagggtctacaggtgacaaaactcatacttgtccaccatgcccagcccccgaggcggctggcggccccagcgtattccttttccccccaaaacctaaggacacgcttatgatatctagaaccccggaggtcacatgtgtcgtcgtagacgtaagtcacgaagatcctgaagtcaagtttaactggtacgtcgatggagtcgaggtccataatgctaaaacgaagcctcgcgaagaacagtataattctacctatcgcgtagtctctgtcctcaccgtcttgcatcaagactggttgaacggcaaggagtacaagtgtaaggtttcaaacaaagcccttgccgcgccgatagagaaaacaattagcaaagcgaaggggcagccgagagagccgcaagtgtatacccttcctcctagtagagacgagttgaccaaaaaccaggtgtcacttacatgcctcgtgaaaggcttctacccgagtgatattgcagtcgagtgggaatccaacggccagcccgagaataactacaaaacgacgccgcccgtactggacagtgatggaagtttttttttgtactcaaaactcacggttgacaaaagtcggtggcagcaagggaacgtttttagctgctctgtcctccatgaagcactccattctcattatacccagaagtctctgtctctctcccctggtaagggaggttctgacagttggcaggaagaggtaataaaactctgcggtcgagagcttgttcgagcacaaattgctatatgtggaaaatctaccgcttcagacgccgccggagctgatgcggatgccggggctcgccagctctatagcgccttggccaacaaatgttgtcacgttggctgcacgaagcgctccctggctcagttttgc

SEQ ID NO: 197 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatggcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcacgttgggtgtaccaagcggtcccttgcgcaattctgc

SEQ ID NO: 198 - DNA, 945 bp, synthetic construct
  source 1..945: mol_type = other DNA
atggagactgatacacttcttctctgggtcctcctcctctgggttccaggctcaacaggtgataaaactcatacttgccccccctgccccgcgcctgaagctgcaggggggccatcagtcttcttgtttccaccaaaacctaaggatactctcatgattagccggacccctgaggtgacatgtgttgtggtcgatgtatctcatgaagatcccgaagtaaaatttaactggtacgtagacggggttgaagttcataacgcgaaaacgaaacctcgggaggagcaatataatagcacgtatagagttgtttcagtccttacagttctccaccaagactggctgaatggcaaggagtataagtgtaaagtatccaataaagccttggctgcgccaatcgagaagacgatcagcaaagccaaaggtcagcctcgcgaaccgcaggtctatacattgcccccttcacgcgacgaactcacgaaaaatcaagtctctttgacttgccttgtgaaaggcttctacccctccgatattgccgtcgaatgggaaagcaatggacagccggaaaataattacaaaacgacaccccccgtgttggattccgatgggtccttcttcctctattccaagctgacggtcgataagtctcgatggcagcagggaaatgtcttctcttgctccgtccttcatgaggcattgcacagccattatactcaaaagagtctctctctgtctccaggcaaagggggttccgactcttggcaagaagaggtcataaaactgtgcggccgggagctcgtcagagcgcagatcgctatatgtggaaaatccaccgcgagtgacgcagcaggtgcacaagccgacgcaggagctaggcaactgtactcagcccttgccaataagtgttgtcacgtaggttgtactaaacgctccctggcacaattttgt

SEQ ID NO: 199 - DNA, 945 bp, synthetic construct
  source 1..945: mol_type = other DNA
atggaaaccgataccttgctcctttgggttctgctcctttgggtccctggaagtacaggggataagacacacacgtgcccaccctgccctgccccagaggcagccggaggtcctagtgtgtttctgttccccccaaagcccaaggacacccttatgatatctaggacaccagaagttacgtgcgtcgttgtggacgttagccacgaagacccagaagtgaagtttaattggtacgttgatggagtcgaagtgcacaatgcaaaaacaaaaccacgagaagagcagtataacagtacttatagagtagtcagcgtccttactgtattgcatcaggattggctgaatgggaaggaatataaatgtaaggttagcaataaagcccttgcggctcctatcgagaaaactattagcaaagcaaagggccaaccccgagagccccaagtttatacactgccacccagtcgagatgagctgactaaaaatcaagtatccctgacctgcttggttaaggggttttatcctagtgacatcgcggttgagtgggaatccaacggccaaccggagaataattacaaaaccacgccacctgtattggattccgatggtagcttctttctctatagtaaacttacagtcgataagtcaagatggcagcagggaaacgtattttcatgctcagttctgcatgaggccttgcactcccattacactcaaaaatcactgagcctcagtcctggtaagggtggctctgactcatggcaggaggaagtaatcaagctgtgtgggagggaattggtaagggctcagattgcaatttgtggaaagagcacagcgtctgacgctgcaggtgccgacgcacaggcgggcgcgaggcagctctacagtgctcttgcgaacaagtgttgtcatgtaggttgcacgaaacgaagtttggcgcaattctgt

SEQ ID NO: 200 - DNA, 945 bp, synthetic construct
  source 1..945: mol_type = other DNA
atggagactgacactctccttctttgggtgctgttgctctgggttcccggaagcacgggggataaaacacacacctgccccccttgcccagcacctgaagcagcgggtggtcccagcgtttttcttttcccccctaagccaaaggacacgctcatgataagtcgcaccccggaggtcacctgcgtcgttgttgacgtatcacatgaagatcctgaggtgaagtttaattggtacgtagatggtgttgaggtccacaacgcaaagacgaaaccgagggaagaacagtacaacagtacttatcgcgtagtctccgttctgactgtcctgcatcaagattggttgaacgggaaggagtacaagtgcaaagttagtaacaaggctctcgcggccccaattgagaagacgatatccaaagcgaaaggacagccgagagagccccaagtctacactctgcccccttccagggatgagctcaccaaaaatcaggtcagtctcacgtgcctggttaagggattctacccaagtgatatagcagttgaatgggagagtaacggccagcccgagaacaactataaaactacaccgcccgttcttgattccgatgggtctttcttcctttatagtaagctcaccgttgataagtcccgatggcagcaaggtaatgtcttctcatgttcagttcttcatgaagccctgcattcccattatacacaaaagagcttgtccttgtcaccgggcaaaggcggtagcgattcttggcaagaagaagttataaagttgtgcggtagggaactggtacgcgctcaaatagctatatgcggtaagtctactgcttcagatgcggctggcgcacaggcacaggccggtgctagacaactctatagtgcgctggccaacaagtgctgccatgtggggtgtacaaaacggagtcttgcccagttttgt

SEQ ID NO: 201 - DNA, 945 bp, synthetic construct
  source 1..945: mol_type = other DNA
atggaaactgacacgcttttgctttgggtcctccttctttgggttcccggctctactggagacaaaactcatacatgccccccatgcccagcacccgaagcggccggaggtccgtctgtctttctgtttccgccgaaacctaaagatacgttgatgattagcagaacccctgaggtaacatgtgtggtagtcgatgtctcccatgaggaccccgaggtaaagttcaattggtatgttgacggcgtcgaagtccataacgcaaaaacgaagccccgagaggagcaatataactctacctatcgcgttgtttctgttttgactgtgttgcaccaggattggctcaacggcaaggaatacaaatgtaaagtgtccaacaaggcccttgctgcacctatcgaaaaaacgattagtaaggcaaagggacaaccgcgcgaaccacaggtatatactttgccgcctagcagagatgaactcaccaagaatcaagtttcccttacctgtttggttaaaggattttacccgtctgacatagctgttgaatgggagagcaatggtcagccggaaaataattataaaaccaccccgccagtattggattcagatgggtcctttttcttgtattctaaacttaccgtggataagtctaggtggcaacagggaaacgtcttttcatgtagtgtacttcatgaagccctccatagtcactacacgcagaaatccttgtctcttagtccgggtgaaggtgggtctgattcctggcaggaagaggtgataaagctctgtggtcgggaacttgttagggcgcagatcgctatttgcggcaaatctacagcatcagatgccgccggagctgatgcgaacgcaggagcgaggcagctgtactccgcacttgctaacaagtgttgccatgtcggctgcaccaagaggagtcttgctcaattctgc

SEQ ID NO: 202 - DNA, 945 bp, synthetic construct
  source 1..945: mol_type = other DNA
atggaaaccgacaccctccttctttgggtcttgctgctttgggttcctggttctactggagataagacccatacatgcccgccatgtcccgcacccgaggcagcgggtggaccctctgtctttctgttccctccaaagccaaaagataccctgatgattagccgaaccccggaggtgacttgtgtcgtagtagatgtcagtcacgaggatcccgaagtaaagtttaattggtatgtggacggtgtggaggtacataacgctaagacgaaaccccgagaggaacaatacaactctacgtacagggtcgtctcagtgctcacggtcctgcaccaggactggcttaatgggaaggaatataaatgcaaagtctctaataaggcgcttgctgcacctattgaaaaaacgatttctaaggcgaagggacaaccccgggagccacaagtctacacccttcctccaagcagagatgagcttacgaaaaatcaagtgtctcttacgtgcctcgtaaagggcttttacccatccgacattgcggtggagtgggaatcaaacgggcagccggaaaataactacaaaacaacgccgcctgtattggattccgacggctctttcttcctttacagcaaactgacagtcgataaatccagatggcaacaagggaacgttttttcatgttccgttctgcatgaagcccttcacagtcattacacccaaaagtcactttcactttcaccgggcgagggggggtcagactcctggcaagaggaagttataaagttgtgcggcagggaactggttagagcgcagatagcgatttgcggaaaatctactgcgagtgatgctgcgggagcgaatgcggacgccggggcccgacagctctattccgcactcgccaataagtgctgccatgttggttgtacgaagagaagtcttgcacaattttgc

SEQ ID NO: 203 - DNA, 945 bp, synthetic construct
  source 1..945: mol_type = other DNA
atggaaacagacacgctgcttttgtgggtactgttgctttgggtcccaggatccacaggtgataagacacatacatgccccccctgcccggctcccgaagctgccgggggaccgtcagtgtttttgtttccgcccaagccgaaggatactttgatgattagtcggacaccagaagtgacatgtgttgtcgttgacgtgagtcacgaggatcccgaggtcaagttcaactggtacgttgatggggttgaagttcacaacgctaaaacgaaaccccgcgaagagcagtataactccacttaccgggtcgtcagtgtcctgacggtcttgcaccaggactggctgaatggaaaggaatacaagtgtaaagtttccaataaagcactggccgccccgatcgaaaaaacaatttccaaagctaagggacagcccagggaaccgcaagtttatactcttccaccctcccgggatgaactgaccaaaaaccaagtgtctttgacgtgcctcgtaaagggcttctacccgtcagacatagctgtcgaatgggagtctaatggacagccggaaaacaattataagactacaccgccggtgcttgatagtgatggaagtttctttttgtactccaaacttacggtcgataaaagccggtggcagcagggaaacgtattcagttgtagcgttctgcatgaagctcttcattctcactacacccagaagtctctgtctctgagccccggagagggtggatctgattcttggcaggaagaagtgataaagttgtgcggccgggaattggtacgcgcccagatagccatttgcgggaagtctacggcgagtgacgcagcaggtgctgacgcggacgctggtgctagacagctgtattctgccctggctaataagtgttgccacgttggctgcaccaagagatccctggcccaattctgt

SEQ ID NO: 204 - DNA, 945 bp, synthetic construct
  source 1..945: mol_type = other DNA
atggaaactgacactttgctgttgtgggtcctcctgttgtgggtccccggaagtacaggagacaagacacatacttgtcccccctgcccagctccagaagctgccggagggccgtcagtcttccttttccctccaaaacctaaggatacgcttatgatttctcgaacgccagaggttacgtgtgtagtcgtggacgtttcccacgaggatcctgaggtcaagtttaactggtatgtagacggggttgaggtccataatgccaagacaaagccgcgcgaggaacaatacaacagtacatatagggtggtgagcgtcctcacagtcttgcatcaagattggctcaacggcaaagagtacaaatgtaaggttagcaacaaagccctcgctgctcccatcgaaaagacgatttctaaggcgaagggccaaccacgagaaccgcaagtatatactcttcccccttcacgggacgagctgaccaaaaaccaggtatccttgacttgcctggtcaaaggattttacccctctgatattgcggtcgagtgggagagtaatgggcaaccagaaaataattataaaacgacccccccggtactcgacagtgatgggtcttttttcctgtattctaagcttacggttgataagtctagatggcagcaagggaatgtcttctcatgtagtgttctgcatgaagcacttcattctcactatactcagaaatctctttcccttagtccgggagaaggtgggagcgatagttggcaagaggaggtgataaaactgtgtggtcgggagctggtgagagcccaaatagctatctgcggcaaatcaacagcaagtgatgcggcaggagcggaagcggaggcgggagcgcggcaattgtatagtgcccttgctaataaatgctgtcacgttgggtgtactaaacgatctcttgctcaattctgc

SEQ ID NO: 205 - DNA, 945 bp, synthetic construct
  source 1..945: mol_type = other DNA
atggagacggatacgttgttgctttgggtcctcctgttgtgggtgcccggctctaccggtgataaaacccatacatgtcctccgtgtcccgctccagaagccgctggcgggccatctgtgtttttgttcccccccaagcctaaggatacgttgatgatcagcaggaccccggaggttacatgcgtagtagttgacgtttctcatgaagacccagaagtaaaatttaactggtatgtcgatggcgtcgaagtacataatgctaaaactaagcccagggaagagcaatacaattcaacgtaccgagttgtgagtgtccttacggtcctgcaccaagactggttgaacggcaaagagtacaaatgcaaagtgtctaacaaggcattggccgcgcctatagaaaagaccattagcaaagcaaaagggcagcctcgggaaccccaggtctacacgctgccaccttcccgagatgaattgacgaaaaaccaggtctctttgacctgcttggttaaaggcttctacccaagcgacattgcagtggagtgggagtctaacgggcaacccgaaaacaactataagacgactccccctgttcttgattctgatgggagtttttttctgtacagtaagttgacagtggataaatcaagatggcagcaaggtaatgtcttctcttgttcagtgcttcacgaagcattgcattctcactacacacaaaagtctttgtccttgtctccaggtgaaggcggtagcgattcatggcaagaagaagtcattaagctgtgtggaagggaactggttagggcccaaattgcgatatgtggaaagtctacggcgagtgatgcggccggtgctcaagcggatgcgggtgctagacagttgtactcagcccttgcgaacaaatgttgtcacgttggctgtacgaaacgcagccttgctcaattctgc

SEQ ID NO: 206 - DNA, 945 bp, synthetic construct
  source 1..945: mol_type = other DNA
atggaaactgacacccttctgctctgggtactcttgttgtgggtccccggcagtacaggcgataaaactcacacatgccccccatgcccagcaccggaagctgccggaggaccgtctgtattcctctttccgcccaaaccgaaagacacgttgatgatttctcggactcccgaggtaacttgtgtcgtggtcgacgtctcacacgaggacccggaggtcaaatttaactggtatgtcgatggggtggaggtccataatgctaagacgaagcccagagaagaacagtataactctacttatagagttgtaagcgtgctcactgtattgcaccaggactggctcaacgggaaagaatataagtgtaaggtctcaaacaaagctctcgcagccccgatagagaaaacaatatctaaggccaagggccaaccgcgcgagccgcaggtttatacacttccaccctcccgcgatgagctgaccaagaaccaggtctctctcacctgtctcgtaaagggcttttatccctccgacattgcagtggagtgggaatcaaacggccagccggaaaataattacaagaccactcctcccgtcctcgactccgatgggtcatttttcctgtacagtaagctcaccgttgataagtcaaggtggcagcagggcaacgtgtttagctgtagtgttctgcatgaggcgctccacagtcactacacccagaaaagtctgagcctttccccaggtgagggtggtagcgatagctggcaggaggaagtaattaaactctgcggtagagaattggtaagggcccaaattgccatctgcggaaagagcaccgcatcagatgctgcgggcgcggatgcgcaggctggtgctaggcaactctactctgccctggcgaataaatgttgccacgtcggttgcacgaaacgaagtttggctcaattttgc

SEQ ID NO: 207 - DNA, 945 bp, synthetic construct
  source 1..945: mol_type = other DNA
atggaaacggacacacttctcttgtgggttcttctgctctgggttcccggcagtacaggcgacaaaacacatacatgccccccttgcccggctcccgaggccgccggtggtcctagcgtctttcttttccctcccaaacccaaagacacacttatgattagcagaactcccgaggtaacatgtgtggtcgtagacgtaagtcacgaagatcccgaagttaaattcaactggtacgttgatggtgtggaagttcataatgcaaaaaccaaaccgcgagaggaacagtataactctacctaccgcgtggtctcagtgctgactgtcctgcatcaggactggctcaacgggaaggaatataagtgcaaagtgagtaataaggcccttgcagctcccatagaaaagacgatatcaaaggctaaaggacagccgagggagccacaggtgtacactttgcctccgagtagagatgaactcactaaaaaccaagtaagtttgacatgcctggtcaaaggtttttaccccagtgatatagcggttgagtgggagtccaatgggcaaccggagaacaactataagactactccacctgtcctggatagcgatggaagtttttttctttactcaaagctgacggtggataagagtcgatggcagcagggcaatgtgtttagctgttctgtgcttcacgaagcacttcactctcattatacccagaagtcattgagcctttcccctggtgaaggagggtcagattcctggcaggaggaggttataaagctgtgtggccgggaactcgtgcgagctcaaattgcgatctgtggaaaatccaccgctagtgatgcggcgggagcacaagctcaagcgggcgctcgacaactttatagcgctttggctaataagtgctgccatgtgggttgtacaaagcgcagcctcgctcaattttgc

SEQ ID NO: 208 - DNA, 945 bp, synthetic construct
  source 1..945: mol_type = other DNA
atggaaacagacactctcctcctgtgggtacttctcttgtgggtaccaggatccaccggggataagacgcacacttgccctccttgcccggcacccgaagccgctggtgggcctagtgtattcctgttccccccgaagccgaaggatactcttatgatttcacgcacgcccgaggttacatgcgtagtagtggacgtatctcacgaagatcccgaagtcaagttcaattggtatgtcgacggagtagaagttcacaacgcaaagacaaaaccgcgggaagagcaatacaactccacgtaccgcgtcgtttctgttcttacggtcttgcaccaggactggctcaatggcaaggagtataagtgcaaggtatccaacaaggcccttgccgcacctattgaaaagactatcagcaaggccaagggacagccaagggagcctcaagtctacacgctcccgcctagtagagacgagttgacaaagaatcaagtgagtttgacttgtctggttaaaggtttttacccgtcagatattgcagtagaatgggaatctaacggacaacccgaaaacaactataaaacgacgcctcctgtgttggattcagatgggtcattttttctctactcaaagctcacggtagataaatcaagatggcaacaaggcaatgtattttcctgctccgtgctccacgaggctctgcacagccattatacgcaaaagagtctgtctttgagcccaggtgagggtggctccgattcctggcaggaggaagtaattaagttgtgcggcagggaacttgttcgcgcacaaatagccatttgtggtcagagcacagcatcagatgccgccggagccgacgccaacgcaggtgcccgccaactttattctgccctcgcaaacaaatgctgccacgtcggctgcacgaagaggagcctcgcccaattttgc

SEQ ID NO: 209 - DNA, 945 bp, synthetic construct
  source 1..945: mol_type = other DNA
atggaaacagacacgctgctcctctgggtgttgcttctctgggtgcctggcagtacaggtgataagacccatacgtgcccgccatgtccagcccccgaggcagccggaggtccttccgttttccttttcccccctaagcccaaggacactctgatgatctcccggacgcctgaagtcacttgcgtagtcgtagacgtttcacatgaggatccagaagttaaatttaactggtacgtcgatggcgtcgaggtccataacgcgaaaaccaagcccagggaggaacaatataactccacctatagggtcgtgagtgtgctcaccgttttgcaccaagactggctcaacgggaaagagtacaaatgtaaagtttcaaataaggctttggccgccccaatagagaagactatatccaaggctaagggacagcctcgagaaccgcaggtatatacgcttcctccgtctagggatgaactcacaaaaaaccaggtttctttgacctgcttggtaaagggattttatccctccgacattgcggtcgaatgggagagcaacggacagccggaaaacaattacaaaacgacacccccggttttggactctgatggaagcttcttcctctatagtaagttgaccgtagacaagtctcgctggcagcagggaaacgtcttcagttgctcagttctccatgaggcgttgcatagtcactatacacagaagagtcttagtttgtctccaggagaaggaggttctgattcttggcaagaggaggtaatcaaattgtgtggccgagaacttgttagagctcagatagccatctgcggacagtctacggcgtccgatgcggccggagctaatgctgacgcaggtgcgcgacagctgtactccgcactggcgaataagtgctgccacgtgggatgcactaagcggtctctcgcgcaattctgt

SEQ ID NO: 210 - DNA, 945 bp, synthetic construct
  source 1..945: mol_type = other DNA
atggagaccgacactcttttgctctgggtgctgttgctgtgggttccaggttcaacgggagataaaacccacacctgtccaccatgcccggcgccggaagccgccgggggacccagcgtatttcttttcccccccaagcccaaagacacgctgatgatttcacgaacgccggaggtgacttgcgtggtagtggacgtctcccatgaggatcccgaagttaaatttaattggtatgtagatggtgttgaggtccataatgctaaaacaaagccgcgggaagagcaatataactccacctatagagtggtctctgtactcactgtcctgcaccaggattggctgaatgggaaagagtacaagtgtaaagttagcaacaaagcgctcgccgcgcctatcgaaaaaacgatttccaaagcaaagggccaaccacgagaaccccaggtttacaccctgccacccagtcgagatgaactcactaagaatcaggtgtcccttacatgcctcgtcaagggattctatccgagcgatatagcggtggaatgggagagtaacggtcaacccgaaaataactataaaaccactccgccggtactcgattctgacggttccttctttctttattccaaactgactgtagacaaatcacggtggcagcagggcaacgtgtttagctgctctgtactccatgaggccttgcattctcattatactcaaaagagtctgagtctgagtccaggtgaagggggttccgattcatggcaagaggaagtcattaaactctgcggaagggaacttgtaagagcacaaatcgcgatttgtgggcaatctaccgcatccgacgcggctggagcagatgcagatgccggagcgaggcagctgtattcagcattggctaacaaatgttgccatgttggatgtacgaagagatcacttgcacagttctgt

SEQ ID NO: 211 - DNA, 945 bp, synthetic construct
  source 1..945: mol_type = other DNA
atggaaactgacaccctccttctctgggtactgctcttgtgggttccaggctctactggcgataagactcatacctgcccgccctgtcccgcacccgaggctgccggagggccatcagtgttccttttcccaccaaagccgaaggatacacttatgatcagcaggacacccgaagtgacctgtgtagtcgtagacgtgtcccacgaagaccccgaagtaaaatttaattggtatgtcgatggcgtagaggtccacaacgcgaaaacgaaaccccgcgaagaacaatataattccacataccgagttgtcagcgtcctcactgttctccatcaggactggctgaatgggaaggaatataagtgcaaggtctcaaacaaggcgctggcggcccccatagagaaaacgatttctaaggccaaaggacagccacgggaaccgcaggtctatacgctcccacctagtagggatgagttgaccaagaatcaggtatccctcacatgtctcgtcaagggattctatcccagcgacatagccgtggagtgggaatctaacggtcaacctgagaataactataaaacaacccccccggtcctcgactccgatggtagcttctttctgtattccaaactgacggtagataaaagccgatggcaacagggtaacgtctttagttgttctgtattgcacgaggcgctccatagtcactacacacagaagtctttgagcctctcacctggggaggggggtagcgattcttggcaggaggaagtgatcaaactgtgcggcagggaactggtcagagcacagatagcaatatgcggtcagagtacggcctctgacgccgccggtgcggaggctgaggcaggggcgagacagctctacagcgctcttgcaaataagtgttgtcacgtggggtgcacaaagagatccttggcgcaattttgt

SEQ ID NO: 212 - DNA, 945 bp, synthetic construct
  source 1..945: mol_type = other DNA
atggaaaccgatacattgcttttgtgggtcttgttgctgtgggtgcccggttctactggtgataagacccacacatgtccgccatgtccagccccagaggcagcagggggcccgtccgtattcttgtttcccccgaaacccaaagatacccttatgattagtcgaactccagaagtcacgtgtgtggtggtggacgtatcccacgaggaccccgaagtgaaattcaattggtatgtggacggggtggaagtccataacgctaagacgaagcccagagaggagcagtacaattctacctatcgggttgtatctgtgcttactgttctccatcaagattggctgaacgggaaggaatacaaatgtaaagttagtaacaaagcattggcagctcctatcgaaaagacgataagcaaggctaaaggtcaaccccgagagcctcaggtctacactttgccgccctccagggatgagcttaccaagaaccaagtgagcttgacgtgtctcgtgaagggattctacccatcagatatagcggtagaatgggagtctaatgggcagcccgagaacaactataagaccacccctcccgttcttgactccgacggttcctttttcttgtactccaaactcacggtcgacaagtctaggtggcagcaaggcaatgttttcagttgttccgtgctgcacgaagctcttcattctcactatacgcaaaaaagcctgagtctttcacctggagaggggggttccgattcttggcaggaagaagtcattaagctgtgcggcagagaacttgtgcgcgcacaaattgctatttgtggacagtcaactgcatctgacgccgctggagcccaagcggacgcaggggcaaggcagctttattcagcgcttgcgaataagtgttgccatgtgggttgcacgaaacgaagcctggcgcaattttgt

SEQ ID NO: 213 - DNA, 945 bp, synthetic construct
  source 1..945: mol_type = other DNA
atggagactgatacattgttgctttgggtactcttgctttgggtgcccggaagtacaggggataagacacatacatgtcctccctgtcccgctccggaggcagccggtgggccttcagttttcttgtttccgccgaagcctaaggacacgttgatgatatcccgaacaccagaggtcacatgcgtcgtcgtggacgtctcacacgaggaccctgaagtgaaattcaactggtatgtagacggggtcgaagttcacaatgcgaaaactaaacctcgcgaggagcaatataactcaacataccgcgtagtgtccgtcttgactgtccttcatcaggattggctgaatggtaaagaatataaatgtaaagtttctaataaagcgcttgcggcacccattgagaagacaatttccaaagccaaaggccaaccccgagagcctcaggtatatacgctgcctccgtctcgagatgagttgacaaaaaatcaagtcagcttgacttgtcttgtaaaggggttctatccgtcagacatagcagtggagtgggaatccaacgggcaaccagaaaataattacaaaaccactccgcccgtgcttgactcagatgggagcttcttcctttatagcaaacttacggtagataaatccagatggcagcaaggcaacgtattcagctgtagtgtgctgcatgaagcgcttcactcccattatactcaaaaatctctttctctgtcaccgggcgagggcggaagtgatagttggcaggaagaggtcatcaagctctgtgggagagagcttgtacgcgctcagattgctatatgcggccagtcaactgcaagcgatgcagcgggtgccgatgcccaagcgggggcacggcaactctactcagccctcgcgaataaatgttgtcatgtagggtgtactaagagaagcctcgcgcaattttgt

SEQ ID NO: 214 - DNA, 945 bp, synthetic construct
  source 1..945: mol_type = other DNA
atggaaactgatactcttcttttgtgggtactgttgttgtgggtcccaggaagtaccggcgataaaacgcatacttgcccgccgtgcccagcacctgaggcagccggcggccctagtgtcttcttgttcccgcccaagcccaaggatacactcatgatctcccgaacgccagaggtcacatgcgtagttgttgacgtttcccatgaggaccctgaagtgaaatttaactggtacgtcgacggcgttgaggttcacaacgctaagactaagccaagagaggaacagtacaattcaacttatagagtggtgtctgtattgacagttctccatcaggattggctgaacggaaaagaatataagtgcaaggtctcaaataaggcgctcgctgcacccatagaaaaaaccatatcaaaagcgaaggggcaaccaagagaaccccaggtgtacacgctccccccgtccagagatgaactcacgaagaatcaagtgtcactcacatgtcttgtaaaggggttctacccctctgatattgccgtagaatgggaaagcaacggacagcccgagaataactacaagacgacaccgccagttcttgattctgacggaagctttttcctctattcaaaattgaccgttgacaagtcccgatggcaacagggcaacgttttctcatgctccgtccttcacgaagccttgcattcccactatacgcagaagagtctctctttgagccccggagagggaggcagtgattcatggcaagaggaagtgatcaaactttgcggcagagaattggttagagcccagattgccatttgtggacaaagtacggcctcagatgctgcgggggcacaagctcaggcgggcgcacgccagttgtacagtgctctggcgaataagtgctgccacgttggttgcaccaagcgatccttggcgcaattttgc

SEQ ID NO: 215 - DNA, 945 bp, synthetic construct
  source 1..945: mol_type = other DNA
atggaaacagatacgctccttttgtgggtactgttgttgtgggtgcccggctctacgggcgacaagactcatacttgtccgccctgccccgctcctgaggctgccggaggcccttcagtattcttgtttccgccgaaaccgaaggataccttgatgattagtaggacaccggaagtcacctgcgtagtggtggacgtaagccacgaagatcccgaagtaaagtttaattggtatgttgatggcgtagaggtgcataatgcgaaaaccaaacctagggaggaacagtacaatagtacttaccgcgtagtgtcagtgcttaccgtgctgcatcaggactggcttaatgggaaggaatacaaatgtaaagtatccaataaagcgctggcggctcccatcgagaaaacgatctcaaaagccaaaggacaaccacgggaaccgcaggtctatactctgccaccttcaagagacgaacttaccaagaaccaagtctcattgacgtgcttggtaaaaggtttttatccgtctgacatcgctgttgaatgggagtctaacggccagccggagaacaattacaaaacaactccaccagtcttggattcagatgggtctttttttttgtattcaaagcttaccgttgacaaaagccgctggcaacaaggaaacgttttcagctgcagtgtgctgcacgaagcgctccacagtcattatacccagaaatctttgagcctgtctccaggggaaggtgggagtgactcttggcaagaagaggttatcaaactttgcgggcgggagctggtaagggcccaaattgcaatatgcggcaaaagtactgcatctgatgccgctggggccgatgctaacgcgggcgcaagacaactttatagcgcgttggcgaacaaatgctgtcatgtgggatgcaccaaacaaagtttggcgcaattttgt

SEQ ID NO: 216 - DNA, 945 bp, synthetic construct
  source 1..945: mol_type = other DNA
atggaaaccgataccttgctgctttgggtgctcttgctgtgggttcccggttccacgggtgataaaactcacacgtgtccgccatgccccgcacctgaagcggcgggtggtccgagcgtgtttttgtttccgcctaagcccaaggataccctgatgattagtcggacacccgaagtaacatgtgtcgtcgtggatgtaagtcacgaggatcccgaagtgaaattcaactggtatgtggatggagttgaagtccataatgcgaaaacaaaaccgagagaggaacagtacaactcaacataccgggtggtaagtgtactgacggtactccaccaggactggctgaatggtaaggagtacaaatgcaaagtttcaaataaggcgctcgctgcccccatcgagaaaaccattagtaaggctaaaggtcaacctagggagccacaagtatatacattgccgccttctagagatgagctgaccaaaaaccaggtcagcctgacctgtttggtgaaaggcttctatccaagcgacattgctgtcgagtgggagtcaaatgggcagccggaaaataactataaaacgactcctcctgttctcgactccgatggttcattcttcctctactcaaagcttaccgtggataaatccaggtggcaacaaggtaacgtgttctcatgttccgttctgcacgaagcactgcattcccattatacacaaaaatccctgagcctctcacctggggagggcggaagcgatagttggcaagaggaagtaataaagctgtgtggcagggaactcgtaagggctcagattgcgatatgtggaaaaagcactgcttctgacgccgcaggggccaacgcagatgctggcgcccgacaactctattctgcgcttgcgaacaagtgttgtcatgtaggatgtaccaagcaaagccttgctcagttctgt

SEQ ID NO: 217 - DNA, 945 bp, synthetic construct
  source 1..945: mol_type = other DNA
atggagaccgatacactcttgctctgggtccttcttctttgggttccaggctccacaggagacaaaacccacacttgtccgccctgtcccgctccggaggctgcaggcggcccaagtgtgtttcttttccccccaaagccgaaagacaccttgatgatatcccgcacacccgaagtgacttgcgttgtcgtcgacgtgtctcatgaggacccagaagtcaagtttaattggtacgttgatggcgtggaagttcacaatgcgaaaactaagcccagagaggagcaatataactcaacctaccgggtggtaagtgttctgacagttctccaccaggactggttgaacggaaaagaatacaaatgcaaagtgagtaacaaagccctggctgcccctatcgaaaagaccatatccaaagcgaagggccagccacgggaaccgcaagtatatacacttccaccatctagagatgagcttacaaagaaccaggtgtcccttacctgccttgtcaaaggcttctatccctctgacatcgcagtggagtgggagtccaacggacaaccagagaacaactataagacaacgccgccagtactggattcagatggttcattcttcttgtattctaaactgactgttgataaatcccgatggcagcagggcaacgtttttagttgtagtgttctgcacgaagcccttcattcccattatacacaaaaatctctttccctcagcccaggcgagggaggaagtgacagttggcaagaggaggtgataaagctctgtgggagggagctggtacgcgcacagattgcaatctgcggaaagagcacagcaagcgatgctgctggggccgatgccgatgctggcgctcgacaattgtattcagctcttgctaacaaatgctgtcacgtaggatgcactaaacagagccttgctcaattttgt

SEQ ID NO: 218 - DNA, 945 bp, synthetic construct
  source 1..945: mol_type = other DNA
atggaaaccgacactcttctgctgtgggttcttctcctgtgggtgcctggatcaaccggagataagacccacacatgtccaccatgcccagccccagaagcggcaggtggtccttctgtgtttctctttcctcccaaaccgaaagatactctgatgataagccggaccccagaagttacgtgcgttgtagtagacgtgtctcacgaggacccagaagtgaagtttaactggtatgtcgacggtgtagaagttcataatgcgaaaacaaagcccagggaagaacaatataattcaacgtaccgggtcgtttccgtgctgacagttctgcaccaagattggctcaacgggaaagagtacaaatgcaaagtatcaaataaggccttggctgcgccgattgaaaagacgatttccaaagcaaagggccagccaagggaaccccaggtctataccctccctcctagcagagatgaacttacaaaaaaccaagtctccctcacctgcctggtcaaaggattctatccctcagatatagcagtagaatgggaaagtaacgggcagcccgaaaacaattataagaccactcctccagtactcgattcagacggtagcttctttctgtattccaagctgaccgtagataaaagtaggtggcagcaaggtaatgtcttctcatgtagtgtacttcatgaggcgttgcattcccattacacgcaaaagtctttgagtctcagtccgggtgaaggaggtagcgattcttggcaggaagaagtaattaagctgtgcggccgggagctcgtcagggctcagatagctatatgcggcaagagcacggccagtgatgctgctggtgcagaggctgaagcaggtgccaggcagttgtacagcgcactcgctaataagtgttgccacgtggggtgtacaaagcaatctttggcacaattctgt

SEQ ID NO: 219 - DNA, 945 bp, synthetic construct
  source 1..945: mol_type = other DNA
atggagactgatactttgctgctgtgggttctccttctttgggtcccaggttccacaggagataagacccatacttgtcctccgtgcccggcaccagaggctgcgggtggcccatccgttttcctgtttccgccaaagcctaaggatactctgatgatttcacgcacacccgaagtgacctgcgtggtggtcgacgtatctcacgaagacccagaggtaaaattcaattggtacgtggacggcgtcgaggttcataacgcgaaaactaagccgagagaagagcagtacaactctacgtatcgcgtggtgtccgtactgacagtattgcatcaggactggttgaatggcaaggagtataagtgcaaggtatctaataaggcattggctgccccaatagagaaaacgatcagcaaagcaaaggggcagccgcgcgagccgcaggtatatacacttccaccatcacgggatgagttgacgaaaaatcaagtctctctcacatgtctggtaaaaggtttctatccttctgatatcgccgtggaatgggaaagcaacggccaacccgaaaacaactataagacgacgccgccggtactcgacagcgacggaagctttttcttgtattccaagttgacagtggacaagtctcgatggcagcaaggaaacgtgttctcatgttctgttcttcacgaagcccttcatagccattatactcagaaatctctctcactctccccaggtgaagggggaagtgactcttggcaagaagaagtcattaagctttgcggtcgagaattggttcgggctcaaatagctatttgtggcaagtccacggcaagtgatgcagcgggggctcaggcagacgcgggcgcaaggcagctttattccgcacttgcaaataagtgctgtcacgtcggatgtactaaacaatcacttgcacaattctgc

SEQ ID NO: 220 - DNA, 945 bp, synthetic construct
  source 1..945: mol_type = other DNA
atggagaccgacacacttctcctctgggtcctgctcctttgggtgccaggcagtacaggtgacaagacacatacatgtcccccatgccctgcacccgaagctgctggggggcccagcgtgttcctgtttccgccgaagcccaaggacacattgatgattagtagaaccccagaggtaacttgtgttgtggtcgatgtgtcacatgaagaccccgaggtaaagtttaactggtatgtggatggggtagaggtacataatgcaaaaaccaagccgcgggaggagcagtataattcaacctatcgagtcgtgtcagtcttgaccgtgctccaccaggactggcttaacggtaaggagtataaatgcaaagtcagtaataaggcattggccgcccccattgagaagaccatcagtaaagctaaggggcaacctagagagccacaggtttacaccctccctccctcccgggatgaactcaccaaaaaccaggtgtcccttacttgtttggtaaagggcttttatccttctgatattgctgttgaatgggagtctaacgggcaacctgaaaataactacaaaacaactccccccgttctggactctgatgggtcattcttcctttattcaaaattgacagttgataagagtagatggcaacaaggcaacgtattttcatgttctgtgctccacgaggctctccattcccactacacacagaaaagtctctcactgtccccaggagagggcgggagcgactcttggcaggaagaagtaatcaagttgtgtggcagggaactcgtacgcgctcagattgcaatatgcgggaaatccacggcaagtgacgctgccggggccgacgcgcaagcaggggcacggcagctttactccgccctcgcaaataaatgttgtcatgtgggatgcactaaacagtcccttgcccagttttgc

SEQ ID NO: 221 - DNA, 945 bp, synthetic construct
  source 1..945: mol_type = other DNA
atggagacagatacactgcttctgtgggtgctcttgctttgggtccccggttccacaggcgataaaacccatacctgtccaccatgccccgcgccagaggcagcgggtggtccaagcgttttcctttttccaccgaaaccaaaagatacacttatgatatcaaggacccccgaggtaacgtgcgtcgtagttgacgtttctcacgaagatcccgaggtgaaattcaattggtacgtagatggtgtagaggtacacaatgcgaagacaaaaccgcgggaagagcagtataatagcacatacagagtcgtgagcgtcctcaccgtacttcaccaagattggctgaatggaaaggagtacaaatgtaaggtaagtaataaagcacttgcggcccccatcgagaaaactatcagtaaagcaaaagggcaaccacgagaaccccaggtctacactttgccaccatcacgggatgaactgacaaaaaatcaggtgtcactcacttgccttgttaaagggttctatcctagtgacatagcggtagaatgggagtctaacgggcagcctgagaacaattataaaactacgccccctgttcttgattccgatggatcattttttctctactccaaactcaccgtagacaaatcccgctggcagcagggcaacgtgtttagttgcagcgttcttcacgaagcacttcactcacattacacacaaaagtccctgagcttgagtcctggggagggtggatctgattcttggcaggaagaagttataaaactttgtggcagagagttggtccgcgcacaaatcgccatatgtggtaaaagcacagcgtctgacgcggcgggagcgcaagcccaggcgggggctcggcaactctactcagccctggctaacaagtgctgtcacgtgggatgcactaaacaaagtctggcgcagttctgc

SEQ ID NO: 222 - DNA, 945 bp, synthetic construct
  source 1..945: mol_type = other DNA
atggagaccgatacgttgctgttgtgggttttgttgctgtgggtacctggatctacaggtgacaaaacccatacgtgtcccccgtgtccggctccagaggctgcgggaggaccgtctgtgttcttgttcccgccgaagcctaaagatacgctgatgattagtcggacccccgaggtgacctgcgtggtagtagacgtatctcatgaagatccggaagtaaagtttaactggtacgtagacggcgtcgaggtacataatgccaagacgaaacccagagaagagcaatataatagcacttatcgagttgtaagcgtattgacggtccttcaccaggactggttgaacggcaaagagtacaaatgtaaggtatccaataaagcattggctgcgccaattgaaaagacaatttccaaagcgaaggggcaacctcgagagccgcaagtctacacgctgccaccgagtagggatgaattgactaagaatcaggtgagtctcacgtgtctcgtgaaggggttttaccccagtgatattgcggtagaatgggagtccaacggtcagccagaaaataattataaaacaacgccccctgtattggattctgacgggagctttttcctgtactcaaaactcaccgtagataagagtcgctggcaacagggcaacgtattctcatgtagcgttctgcacgaggcgctgcactctcactacacacagaagagtttgagtttgtcccctggcgaaggaggttctgattcctggcaggaggaggtgattaagctgtgtggccgcgaattggtgagggctcaaattgctatttgcggacagagcacagcgtccgatgccgccggcgcagatgctaatgccggtgcaaggcaactgtactccgctctcgccaataagtgttgtcatgtcggctgcaccaagcaatccctggcccagttttgc

SEQ ID NO: 223 - DNA, 945 bp, synthetic construct
  source 1..945: mol_type = other DNA
atggagacagatacgctcttgctgtgggtactcctcctctgggtcccaggctcaacgggcgacaagactcacacttgtcccccatgtccagcaccggaagctgccggcggtccctcagttttccttttcccccccaaacccaaggacacccttatgatttcaaggacaccagaggtaacgtgcgtagtggtggacgtcagtcatgaagacccagaggtaaagtttaactggtacgtggatggggtagaggttcataatgctaaaacaaaaccacgcgaggaacagtacaatagtacgtatagagtggtctccgttcttacggtgctgcatcaggactggctgaacggaaaagagtacaagtgtaaggttagcaataaggcgctggcggccccaatcgaaaagacgatttctaaggccaaaggccagccaagggagccacaagtatatacccttcccccttcccgagatgagctgactaagaatcaagtcagtctcacctgccttgtcaaagggttctacccatccgatattgctgttgaatgggagtctaatggccagccggagaacaattacaagacaactccgcctgtattggattccgacgggtcttttttcctctattcaaaactcacagtagacaaaagtcgatggcagcaaggtaacgtgttttcttgctctgtgttgcatgaagcacttcattctcattatactcaaaaatcattgagcctcagtccaggcgaagggggtagtgactcatggcaggaggaggtaatcaagctttgcggacgagagttggtcagggcccagatagctatttgtggtcagtccacggcgagtgacgcagcaggggcgaatgccgatgcaggagcaagacaactgtattctgctctggccaacaagtgttgtcatgtagggtgtactaaacaaagtctcgcccagttctgc

SEQ ID NO: 224 - DNA, 945 bp, synthetic construct
  source 1..945: mol_type = other DNA
atggaaacggatacgctgttgttgtgggtcttgctcctgtgggtccccggatccacaggtgataaaacccacacctgtcccccatgtccggctcccgaagcagcggggggcccttcagtttttctctttccccccaaaccgaaagacacgctgatgattagcagaactccagaggttacctgtgtagttgtggacgtttcacacgaggatcccgaggttaaattcaactggtatgtggacggcgtcgaagtgcataatgcaaaaacaaagccccgggaagaacaatataatagtacctatagggtcgtttccgtactgaccgtacttcatcaagattggctcaatgggaaggaatacaaatgtaaagtgagtaataaagccctggcggcaccgatcgaaaaaaccatttcaaaggctaagggacaaccgcgcgaacctcaggtctataccttgcccccttcacgcgacgagcttacgaagaatcaggtaagccttacttgtcttgtcaagggtttttaccccagcgacatagctgtcgagtgggaatccaatggccaaccggagaataattacaaaactacccctcctgttcttgatagcgacggaagcttcttcttgtattccaaactcacagtagataaaagtaggtggcagcagggcaatgtattttcttgcagcgtcctgcatgaagcactgcatagccattatactcaaaagtccctgtctttgtctcctggagagggcggaagcgattcttggcaagaggaagttattaagctgtgcgggcgcgaacttgtgagggctcaaatagcgatatgtggtcagagcaccgctagcgatgcggctggtgcagacgccgatgccggtgctaggcaactttacagtgcacttgcgaataagtgctgtcacgtcggatgtactaaacaaagcctcgcccagttctgc

SEQ ID NO: 225 - DNA, 945 bp, synthetic construct
  source 1..945: mol_type = other DNA
atggaaacggatactcttcttttgtgggttcttcttctgtgggtaccaggaagtactggtgataaaacacatacttgccctccttgtccggctcccgaagccgcaggtggaccttccgtctttcttttcccacccaaacctaaagacactttgatgattagccggacccccgaggtaacctgtgtcgtagttgacgtttcccatgaagaccccgaagttaagttcaactggtatgtcgacggcgtcgaggtgcacaacgcgaagactaagccaagagaggagcaatacaattcaacttacagggtcgtgtccgtcttgacagtgcttcatcaagactggcttaatggaaaggaatacaaatgtaaagtctccaacaaggctctcgcagcgcccattgagaaaacgatatccaaagcgaagggtcaaccaagagaaccccaggtttacaccctcccccctagtcgggacgagcttacgaagaaccaggtcagtttgacatgcctggtgaaaggcttctatccgtcagacatcgccgtagagtgggaaagcaacgggcaacccgagaacaactataagacgactcccccggtgttggatagcgatggctctttcttcctgtactctaagctgaccgtagataaatccaggtggcaacaggggaacgtgttttcatgctcagtgctccatgaagccctccattcacactatacacaaaagtctttgtcactgtcccccggtgaaggcggcagtgatagctggcaagaagaagtcataaagctctgtggtcgcgagcttgttagggcccaaattgcgatctgtggtcagtcaacggcttctgacgccgccggagcggaagccgaggcgggtgctcggcaattgtattcagcactggcgaacaaatgttgccatgttggttgtactaaacaaagcctggcccagttttgc

SEQ ID NO: 226 - DNA, 945 bp, synthetic construct
  source 1..945: mol_type = other DNA
atggagaccgataccttgttgctgtgggtgcttcttctctgggttccgggatctacaggagacaagactcacacttgtccaccctgtccagcacctgaagccgctggtggaccatctgtctttctgttcccccctaaaccaaaggatacacttatgatcagcagaacacctgaagtcacatgcgttgtggtagacgtttcccacgaggatcctgaagtgaagtttaactggtacgtggatggcgttgaggttcataatgccaagacgaaacctcgggaggagcagtataattctacttatagggtggtaagcgtactgacagtcctccatcaagactggttgaacgggaaggaatacaagtgtaaagtttccaacaaagctctggcggcgcctatagaaaagacaatatcaaaagcgaaagggcaacccagagagcctcaagtatatacattgccccctagcagagacgaattgacgaaaaatcaggtctctctcacgtgcctcgtgaagggcttctatcctagtgatatagctgtggaatgggaatccaatggacagccagaaaacaactacaagaccacgccccccgtcttggattccgacgggtcattcttcctgtacagcaagctgactgtcgacaagagtcgatggcaacagggcaacgtctttagctgcagcgtcctgcacgaagctctgcatagtcattacacccaaaagtccctttctctctcccctggtgaaggcggttccgattcatggcaagaagaagtaattaagctctgtggacgagagcttgtccgagcacaaattgcgatctgcgggcagagtaccgcatctgacgctgctggcgcgcaggcagatgcgggtgcacggcagctttattcagctctcgccaacaagtgttgtcatgtggggtgtacaaagcagagccttgcccagttttgt

SEQ ID NO: 227 - DNA, 945 bp, synthetic construct
  source 1..945: mol_type = other DNA
atggaaacggacacactgctgctttgggttctcttgctgtgggtcccaggctctacaggggataagacccatacgtgtcccccttgccctgcacccgaggcggctgggggcccttccgtattcttgtttcctcctaagcccaaagataccttgatgataagtcgaacgccagaagtgacttgcgttgttgtggatgtctcccacgaggatccagaagtcaaatttaactggtatgtcgatggggtcgaagtgcataatgctaaaacgaaacccagagaggaacaatacaattcaacataccgcgtagtcagtgttcttactgtgctccatcaggattggctcaatgggaaagaatacaagtgtaaagtctcaaataaagcattggcggcccctatagagaagaccataagcaaggctaaaggtcagcctagggagcctcaagtatataccttgcctcctagcagagatgagttgaccaagaaccaggtcagcctcacatgcctggtgaaagggttttacccatctgatattgccgtcgagtgggaaagtaatgggcagccagagaacaactacaagacgacaccaccggtactggatagtgacggaagtttttttctttacagtaagctcacagtcgacaaaagccggtggcaacaaggaaatgtattttcatgtagcgtacttcatgaagccctccactctcattacacgcagaagtcactttcacttagtccgggtgagggtggaagcgatagctggcaagaggaggttatcaagctctgtggacgagaactcgtgagagcgcaaattgcaatctgcgggcagagcacggcgagtgatgcggccggggcggacgcgcaagcaggagcacgacaactttatagtgctttggctaataaatgttgccacgttggatgtactaaacagagcttggcacagttttgc

SEQ ID NO: 228 - DNA, 945 bp, synthetic construct
  source 1..945: mol_type = other DNA
atggagactgatacactgcttctgtgggtattgttgttgtgggtccccggttccacaggtgataaaacgcacacttgtccgccatgtccggcacctgaggcagcgggaggaccgtccgtgtttctgtttccccctaaaccaaaggacacgctgatgatcagccgaacacctgaagtaacatgcgtggtcgttgacgtgtctcacgaggatccagaagtaaagttcaattggtatgttgacggagttgaagtacataatgctaagactaaaccccgcgaagaacaatataattctacgtacagagttgtatccgtgctcacggtacttcaccaagattggcttaacgggaaagaatataagtgtaaggtctcaaataaggccctggctgctccgatcgaaaaaacgatatcaaaggcaaagggtcaacctcgggagcctcaagtatataccctccccccatctagggatgagctgacaaagaaccaagtttcactgacctgtctcgtaaagggtttctatccttctgacatcgcagttgaatgggagtccaacggccaaccagagaacaactataagacgacaccccccgtgttggacagtgacggaagttttttcctgtactccaagctgacggttgataaaagtagatggcaacaaggaaatgttttcagttgttctgtgttgcacgaggccctccactcacactatacccaaaaaagtttgtctctgagtcccggtgaaggcgggagcgattcatggcaggaggaagtaatcaaactttgtgggcgagaactggtcagggcgcaaatagcgatatgtgggcaaagcacagcttcagatgcagccggtgctcaagctcaggctggagctcgacagctttatagcgccttggctaataaatgttgtcacgttggctgtacgaagcagagcctggcacagttctgc

SEQ ID NO: 229 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcacgttgggtgtaccaagcggtcccttgcgcaattctgc

SEQ ID NO: 230 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatttcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcacgttgggtgtaccaagcggtcccttgcgcaattctgc

SEQ ID NO: 231 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcacttcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcacgttgggtgtaccaagcggtcccttgcgcaattctgc

SEQ ID NO: 232 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcaattcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcacgttgggtgtaccaagcggtcccttgcgcaattctgc

SEQ ID NO: 233 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatggcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgttacgttgggtgtaccaagcggtcccttgcgcaattctgc

SEQ ID NO: 234 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatggcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcttgttgggtgtaccaagcggtcccttgcgcaattctgc

SEQ ID NO: 235 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatggcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcaagttgggtgtaccaagcggtcccttgcgcaattctgc

SEQ ID NO: 236 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatggcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtaaagttgggtgtaccaagcggtcccttgcgcaattctgc

SEQ ID NO: 237 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgttacgttgggtgtaccaagcggtcccttgcgcaattctgc

SEQ ID NO: 238 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtaaagttgggtgtaccaagcggtcccttgcgcaattctgc

SEQ ID NO: 239 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacgggaggagaaggctctggaggcgaaggagaagggggcggaagacagctttactctgctctggctaacaagtgttgttacgttgggtgtaccaagcggtcccttgcgcaattctgc

SEQ ID NO: 240 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacgggaggagaaggctctggaggcgaaggagaagggggcggaagacagctttactctgctctggctaacaagtgttgtaaagttgggtgtaccaagcggtcccttgcgcaattctgc

SEQ ID NO: 241 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacgggaggagaaggctctggaggcgaaggatctgggggcggaagacagctttactctgctctggctaacaagtgttgttacgttgggtgtaccaagcggtcccttgcgcaattctgc

SEQ ID NO: 242 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacgggaggagaaggctctggaggcgaaggatctgggggcggaagacagctttactctgctctggctaacaagtgttgtaaagttgggtgtaccaagcggtcccttgcgcaattctgc

SEQ ID NO: 243 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgttacgttgggtgtaccaagcaatcccttgcgcaattctgc

SEQ ID NO: 244 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtaaagttgggtgtaccaagcaatcccttgcgcaattctgc

SEQ ID NO: 245 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaagacagctttactctgctctggctaacaagtgttgttacgttgggtgtaccaagcaatcccttgcgcaattctgc

SEQ ID NO: 246 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaagacagctttactctgctctggctaacaagtgttgtaaagttgggtgtaccaagcaatcccttgcgcaattctgc

SEQ ID NO: 247 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaagacagctttactctgctctggctaacaagtgttgttacgttgggtgtaccaagcaatcccttgcgcaattctgc

SEQ ID NO: 248 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaagacagctttactctgctctggctaacaagtgttgtaaagttgggtgtaccaagcaatcccttgcgcaattctgc

SEQ ID NO: 249 - protein, 29 aa, synthetic construct
DSWMEEVIKLCGRELVRAQIAICGMSTWS

SEQ ID NO: 250 - protein, 27 aa, synthetic construct
  VARIANT 4: note = X is absent or any amino acid
  VARIANT 25: note = X is absent or any amino acid
DSWXEEVIKLCGRELVRAQIAICGXST

SEQ ID NO: 251 - protein, 29 aa, synthetic construct
DSWKEEVIKLCGRELVRAQIAICGKSTAS

SEQ ID NO: 252 - protein, 29 aa, synthetic construct
DSWKEEVIKLCGRELVRAQIAICGKSTWS

SEQ ID NO: 253 - protein, 29 aa, synthetic construct
DSWMEEVIKLCGRELVRAQIAICGKSTAS

SEQ ID NO: 254 - protein, 29 aa, synthetic construct
DSWMEEVIKLCGRELVRAQIAICGKSTWS

SEQ ID NO: 255 - protein, 27 aa, synthetic construct
DSWKEEVIKLCGRELVRAQIAICGKST

SEQ ID NO: 256 - protein, 27 aa, synthetic construct
DSWMEEVIKLCGRELVRAQIAICGKST

SEQ ID NO: 257 - protein, 24 aa, synthetic construct
QLYSALANKCCHVGCTKRSLARFC

SEQ ID NO: 258 - protein, 25 aa, synthetic construct
  VARIANT 1: note = X is absent or any amino acid
  VARIANT 3: note = X is absent or any amino acid
  VARIANT 23: note = X is absent or any amino acid
XQXYSALANKCCHVGCTKRSLAXFC

SEQ ID NO: 259 - protein, 25 aa, synthetic construct
RQLYSALANKCCHVGCTKRSLARFC

SEQ ID NO: 260 - protein, 25 aa, synthetic construct
RQLYSALANKCCHVGCTKRSLAQFC

SEQ ID NO: 261 - protein, 25 aa, synthetic construct
RQLYSALANKCCHVGCTKRSLAEFC

SEQ ID NO: 262-266 - skipped (5 entries)

SEQ ID NO: 267 - protein, 4 aa, synthetic construct
GGGE

SEQ ID NO: 268 - protein, 4 aa, synthetic construct
GEGE

SEQ ID NO: 269 - protein, 4 aa, synthetic construct
GGEG

SEQ ID NO: 270 - protein, 4 aa, synthetic construct
GEGG

SEQ ID NO: 271 - protein, 4 aa, synthetic construct
GGEE

SEQ ID NO: 272 - protein, 13 aa, synthetic construct
GGGEGGGEGGGEG

SEQ ID NO: 273 - protein, 15 aa, synthetic construct
GGGEGGGEGGGEGGG

SEQ ID NO: 274 - protein, 13 aa, synthetic construct
GEGGGEEGGGEGG

SEQ ID NO: 275 - protein, 13 aa, synthetic construct
GGGEEGGGEEGGG

SEQ ID NO: 276 - protein, 13 aa, synthetic construct
GGEGEGGEGEGGS

SEQ ID NO: 277 - protein, 12 aa, synthetic construct
GGGEGGGEGGGE

SEQ ID NO: 278 - protein, 65 aa, synthetic construct
DSWKEEVIKLCGRELVRAQIAICGKSTGGGEGGGEGGGEGRQLYSALANKCCHVGCTKRSLARFC

SEQ ID NO: 279 - protein, 66 aa, synthetic construct
DSWKEEVIKLCGRELVRAQIAICGKSTGGGEGGGEGGGEGGGQLYSALANKCCHVGCTKRSLARFC

SEQ ID NO: 280 - protein, 65 aa, synthetic construct
DSWMEEVIKLCGRELVRAQIAICGKSTGGGEGGGEGGGEGRQLYSALANKCCHVGCTKRSLARFC

SEQ ID NO: 281 - protein, 65 aa, synthetic construct
DSWQEEVIKLCGRELVRAQIAICGKSTGGGEGGGEGGGEGRQLYSALANKCCHVGCTKRSLARFC

SEQ ID NO: 282 - protein, 66 aa, synthetic construct
DSWMEEVIKLCGRELVRAQIAICGKSTGGGEGGGEGGGEGGGQLYSALANKCCHVGCTKRSLARFC

SEQ ID NO: 283 - protein, 66 aa, synthetic construct
DSWQEEVIKLCGRELVRAQIAICGKSTGGGEGGGEGGGEGGGQLYSALANKCCHVGCTKRSLARFC

SEQ ID NO: 284 - protein, 65 aa, synthetic construct
DSWMEEVIKLCGRELVRAQIAICGKSTGGEGGGEEGGGEGGQLYSALANKCCHVGCTKRSLARFC

SEQ ID NO: 285 - protein, 65 aa, synthetic construct
DSWQEEVIKLCGRELVRAQIAICGKSTGGEGGGEEGGGEGGQLYSALANKCCHVGCTKRSLARFC

SEQ ID NO: 286 - protein, 65 aa, synthetic construct; identical to SEQ ID NO: 284

SEQ ID NO: 287 - protein, 65 aa, synthetic construct; identical to SEQ ID NO: 285

SEQ ID NO: 288 - protein, 65 aa, synthetic construct
DSWQEEVIKLCGRELVRAQIAICGKSTGGGEGGGEGGGEGRQLYSALANKCCHVGCTKRSLAQFC

SEQ ID NO: 289 - protein, 65 aa, synthetic construct; identical to SEQ ID NO: 281

SEQ ID NO: 290 - protein, 65 aa, synthetic construct; identical to SEQ ID NO: 288

SEQ ID NO: 291 - protein, 65 aa, synthetic construct
DSWQEEVIKLCGRELVRAQIAICGKSTGGGEEGGGEEGGGRQLYSALANKCCHVGCTKRSLARFC

SEQ ID NO: 292 - protein, 65 aa, synthetic construct
DSWQEEVIKLCGRELVRAQIAICGKSTGGGEEGGGEEGGGRQLYSALANKCCHVGCTKRSLAQFC

SEQ ID NO: 293 - protein, 65 aa, synthetic construct; identical to SEQ ID NO: 291

SEQ ID NO: 294 - protein, 65 aa, synthetic construct; identical to SEQ ID NO: 292

SEQ ID NO: 295 - protein, 65 aa, synthetic construct
DSWQEEVIKLCGRELVRAQIAICGKSTGGEGEGGEGEGGSRQLYSALANKCCHVGCTKRSLAQFC

SEQ ID NO: 296 - protein, 65 aa, synthetic construct
DSWQEEVIKLCGRELVRAQIAICGKSTGGEGEGGEGEGGSRQLYSALANKCCHVGCTKRSLARFC

SEQ ID NO: 297 - protein, 65 aa, synthetic construct; identical to SEQ ID NO: 295

SEQ ID NO: 298 - skipped

SEQ ID NO: 299 - protein, 4 aa, synthetic construct
EGGS

SEQ ID NO: 300 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTGGGEGGGEGGGEGRQLYSALANKCCHVGCTKRSLARFC

SEQ ID NO: 301 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTGGGEGGGEGGGEGGGQLYSALANKCCHVGCTKRSLARFC

SEQ ID NO: 302 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGGEGGGEGGGEGRQLYSALANKCCHVGCTKRSLARFC

SEQ ID NO: 303 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGRQLYSALANKCCHVGCTKRSLARFC

SEQ ID NO: 304 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGRQLYSALANKCCHVGCTKRSLARFC

SEQ ID NO: 305 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGGEGGGEGGGEGGGQLYSALANKCCHVGCTKRSLARFC

SEQ ID NO: 306 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGGGQLYSALANKCCHVGCTKRSLARFC

SEQ ID NO: 307 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGGGQLYSALANKCCHVGCTKRSLARFC

SEQ ID NO: 308 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGEGGGEEGGGEGGQLYSALANKCCHVGCTKRSLARFC

SEQ ID NO: 309 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGEGGGEEGGGEGGQLYSALANKCCHVGCTKRSLARFC

SEQ ID NO: 310 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWMEEVIKLCGRELVRAQIAICGKSTGGEGGGEEGGGEGGQLYSALANKCCHVGCTKRSLARFC

SEQ ID NO: 311 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGEGGGEEGGGEGGQLYSALANKCCHVGCTKRSLARFC

SEQ ID NO: 312 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGRQLYSALANKCCHVGCTKRSLAQFC

SEQ ID NO: 313 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGRQLYSALANKCCHVGCTKRSLAQFC

SEQ ID NO: 314 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEGGGEGGGEGRQLYSALANKCCHVGCTKRSLARFC

SEQ ID NO: 315 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQLYSALANKCCHVGCTKRSLARFC

SEQ ID NO: 316 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEGGGEGGGEGRQLYSALANKCCHVGCTKRSLAQFC

SEQ ID NO: 317 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQLYSALANKCCHVGCTKRSLAQFC

SEQ ID NO: 318 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEEGGGEEGGGRQLYSALANKCCHVGCTKRSLARFC

SEQ ID NO: 319 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEEGGGEEGGGRQLYSALANKCCHVGCTKRSLARFC

SEQ ID NO: 320 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEEGGGEEGGGRQLYSALANKCCHVGCTKRSLAQFC

SEQ ID NO: 321 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEEGGGEEGGGRQLYSALANKCCHVGCTKRSLAQFC

SEQ ID NO: 322 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEEGGGEEGGGRQLYSALANKCCHVGCTKRSLARFC

SEQ ID NO: 323 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEEGGGEEGGGRQLYSALANKCCHVGCTKRSLARFC

SEQ ID NO: 324 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEEGGGEEGGGRQLYSALANKCCHVGCTKRSLAQFC

SEQ ID NO: 325 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEEGGGEEGGGRQLYSALANKCCHVGCTKRSLAQFC

SEQ ID NO: 326 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGEGEGGEGEGGSRQLYSALANKCCHVGCTKRSLAQFC

SEQ ID NO: 327 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGEGGEGEGGSRQLYSALANKCCHVGCTKRSLARFC

SEQ ID NO: 328 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGEGGEGEGGSRQLYSALANKCCHVGCTKRSLAQFC

SEQ ID NO: 329 - protein, 20 aa, synthetic construct
METDTLLLWVLLLWVPGSTG

SEQ ID NO: 330 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTGGGEGGGEGGGEGRQLYSALANKCCHVGCTKRSLARFC

SEQ ID NO: 331 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTGGGEGGGEGGGEGGGQLYSALANKCCHVGCTKRSLARFC

SEQ ID NO: 332 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGGEGGGEGGGEGRQLYSALANKCCHVGCTKRSLARFC

SEQ ID NO: 333 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGRQLYSALANKCCHVGCTKRSLARFC

SEQ ID NO: 334 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGRQLYSALANKCCHVGCTKRSLARFC

SEQ ID NO: 335 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGGEGGGEGGGEGGGQLYSALANKCCHVGCTKRSLARFC

SEQ ID NO: 336 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGGGQLYSALANKCCHVGCTKRSLARFC

SEQ ID NO: 337 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGGGQLYSALANKCCHVGCTKRSLARFC

SEQ ID NO: 338 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGEGGGEEGGGEGGQLYSALANKCCHVGCTKRSLARFC

SEQ ID NO: 339 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGEGGGEEGGGEGGQLYSALANKCCHVGCTKRSLARFC

SEQ ID NO: 340 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWMEEVIKLCGRELVRAQIAICGKSTGGEGGGEEGGGEGGQLYSALANKCCHVGCTKRSLARFC

SEQ ID NO: 341 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGEGGGEEGGGEGGQLYSALANKCCHVGCTKRSLARFC

SEQ ID NO: 342 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGRQLYSALANKCCHVGCTKRSLAQFC

SEQ ID NO: 343 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGRQLYSALANKCCHVGCTKRSLAQFC

SEQ ID NO: 344 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEGGGEGGGEGRQLYSALANKCCHVGCTKRSLARFC

SEQ ID NO: 345 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQLYSALANKCCHVGCTKRSLARFC

SEQ ID NO: 346 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEGGGEGGGEGRQLYSALANKCCHVGCTKRSLAQFC

SEQ ID NO: 347 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQLYSALANKCCHVGCTKRSLAQFC

SEQ ID NO: 348 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEEGGGEEGGGRQLYSALANKCCHVGCTKRSLARFC

SEQ ID NO: 349 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEEGGGEEGGGRQLYSALANKCCHVGCTKRSLARFC

SEQ ID NO: 350 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEEGGGEEGGGRQLYSALANKCCHVGCTKRSLAQFC

SEQ ID NO: 351 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEEGGGEEGGGRQLYSALANKCCHVGCTKRSLAQFC

SEQ ID NO: 352 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEEGGGEEGGGRQLYSALANKCCHVGCTKRSLARFC

SEQ ID NO: 353 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEEGGGEEGGGRQLYSALANKCCHVGCTKRSLARFC

SEQ ID NO: 354 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEEGGGEEGGGRQLYSALANKCCHVGCTKRSLAQFC

SEQ ID NO: 355 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEEGGGEEGGGRQLYSALANKCCHVGCTKRSLAQFC

SEQ ID NO: 356 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGEGEGGEGEGGSRQLYSALANKCCHVGCTKRSLAQFC

SEQ ID NO: 357 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGEGGEGEGGSRQLYSALANKCCHVGCTKRSLARFC

SEQ ID NO: 358 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGEGGEGEGGSRQLYSALANKCCHVGCTKRSLAQFC

SEQ ID NO: 359 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTASDAAGANANAGARQLYSALANKCCHVGCTKRSLAQFC

SEQ ID NO: 360 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTASDAAGANANAGARQLYSALANKCCHVGCTKRSLAEFC

SEQ ID NO: 361 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTGGGEGGGEGGGEGRQLYSALANKCCHVGCTKRSLARFC

SEQ ID NO: 362 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTGGGEGGGEGGGEGGGQLYSALANKCCHVGCTKRSLARFC

SEQ ID NO: 363 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGGEGGGEGGGEGRQLYSALANKCCHVGCTKRSLARFC

SEQ ID NO: 364 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGGEGGGEGGGEGGGQLYSALANKCCHVGCTKRSLARFC

SEQ ID NO: 365 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGEGGGEEGGGEGGQLYSALANKCCHVGCTKRSLARFC

SEQ ID NO: 366 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGEGGGEEGGGEGGQLYSALANKCCHVGCTKRSLARFC

SEQ ID NO: 367 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWMEEVIKLCGRELVRAQIAICGKSTGGEGGGEEGGGEGGQLYSALANKCCHVGCTKRSLARFC

SEQ ID NO: 368 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGGGEEGGGEGGQLYSALANKCCHVGCTKRSLARFC

SEQ ID NO: 369 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTASDAAGANANAGARQLYSALANKCCHVGCTKRSLAQFC

SEQ ID NO: 370 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTASDAAGANANAGARQLYSALANKCCHVGCTKRSLAEFC

SEQ ID NO: 371 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTGGGEGGGEGGGEGRQLYSALANKCCHVGCTKRSLARFC

SEQ ID NO: 372 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTGGGEGGGEGGGEGGGQLYSALANKCCHVGCTKRSLARFC

SEQ ID NO: 373 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGGEGGGEGGGEGRQLYSALANKCCHVGCTKRSLARFC

SEQ ID NO: 374 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGGEGGGEGGGEGGGQLYSALANKCCHVGCTKRSLARFC

SEQ ID NO: 375 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGEGGGEEGGGEGGQLYSALANKCCHVGCTKRSLARFC

SEQ ID NO: 376 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGEGGGEEGGGEGGQLYSALANKCCHVGCTKRSLARFC

SEQ ID NO: 377 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWMEEVIKLCGRELVRAQIAICGKSTGGEGGGEEGGGEGGQLYSALANKCCHVGCTKRSLARFC

SEQ ID NO: 378 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGGGEEGGGEGGQLYSALANKCCHVGCTKRSLARFC

SEQ ID NO: 379 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTASDAAGANANAGARQLYSALANKCCHVGCTKRSLAQFC

SEQ ID NO: 380 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTASDAAGANANAGARQLYSALANKCCHVGCTKRSLAEFC

SEQ ID NO: 381 - DNA, 945 bp, synthetic construct
  source 1..945: mol_type = other DNA
atggagacggacactttgctgctttgggtactgctgctttgggttcctggatctactggcgataaaacacacacgtgtcccccctgcccggctccagaggcggctggtggtcccagcgtattcttgtttcctcccaaacctaaggatacgctcatgatatcccgcaccccagaagttacgtgtgtagtcgtcgacgtcagtcacgaagatccagaggtcaaatttaactggtatgtcgacggagtagaggtccacaatgcgaaaaccaagcccagagaagagcagtacaactccacgtatcgcgtcgtctccgtcctcaccgtactccatcaagattggctgaatgggaaagagtataaatgcaaagtatctaacaaggctctgccagctccgatagaaaagactatatcaaaggccaaggggcagccaagggagcctcaagtctatactttgcccccatctcgggatgagcttacgaaaaaccaggtcagccttacctgtcttgttaaaggtttttatccgagtgacatcgcagtggaatgggaatctaatggtcaacctgaaaacaattacaaaaccacaccgccagtattggacagcgatggtagtttttttctttactcaaaactgactgtagataaaagcagatggcagcagggcaatgtcttttcatgtagcgttatgcatgaggctcttcacaaccactatacccaaaagtcattgtctcttagtcccggaaagggcggaagtgattcttggaaggaggaggtaatcaagttgtgcgggcgagagttggtacgggcacagatcgcgatatgcggaaaatccacaggtgggggcgaaggaggaggtgagggtggaggtgaaggacgacagttgtattccgccttggcaaacaagtgttgccatgtgggttgcacaaaacgcagtcttgcccgcttctgt

SEQ ID NO: 382 - DNA, 948 bp, synthetic construct
  source 1..948: mol_type = other DNA
atggagaccgatacgctgttgctgtgggtattgcttctctgggtgcccggctcaactggggataagacacatacatgccctccctgtccggctccagaggcagccgggggtccatcagtcttcctttttccgcctaaacctaaggatacactgatgatctctcgaacaccggaggtcacttgtgttgtcgttgacgtatcacatgaggatcccgaagtaaagttcaactggtatgtcgatggtgtggaggttcataatgctaaaactaaaccacgggaggagcaatataattccacatatagggtcgtgagcgtgttgacggtgcttcatcaagactggcttaatgggaaggaatataaatgcaaagtgtcaaataaagcacttcctgcgccaatcgagaaaacaattagtaaggcaaaggggcagccgcgagaacctcaggtgtacaccttgccgccttctagagacgagctcacaaagaaccaagtttccctgacttgcctcgttaaggggttttatccgtccgatatagccgtggagtgggagtcaaacggccaaccggaaaataattacaaaacgacacccccagtattggatagtgacggctcttttttcctttattctaagctgactgtggacaaaagccgctggcagcagggcaatgtcttttcatgcagcgtaatgcatgaagccctgcacaaccactacacgcaaaaatccctttccttgtcacccggcaagggcggctctgactcctggaaagaggaagttataaaactctgtggccgagaacttgttcgagctcaaatcgcgatttgtggtaagtcaacgggtgggggcgaaggtggaggcgagggtgggggagaaggaggaggccagttgtactcagctcttgcaaataagtgttgccacgttggttgtacgaagcggagccttgctcgcttctgc

SEQ ID NO: 383 - DNA, 945 bp, synthetic construct
  source 1..945: mol_type = other DNA
atggaaactgatactcttctgctgtgggtcctgctgctgtgggttccaggatctactggagacaaaacacatacttgtccgccttgcccggcacccgaagcggccggcggacccagtgtctttctcttcccacccaaaccgaaagacactctgatgatttccaggacgcctgaagtgacctgcgttgtagttgatgtatcacacgaggatcccgaggtcaagttcaattggtatgtagatggggtggaggtccataatgcaaagacgaagccacgggaggaacagtacaactctacgtacagagttgtcagtgttttgaccgtccttcatcaggattggctgaacggtaaagaatataaatgcaaggttagcaataaagctttgcccgcccctatagagaaaacgatcagtaaggcgaaggggcagcctagggaaccccaggtatataccttgccgccaagtcgagatgagctgacgaagaaccaagtgagtctgacatgcctcgtgaagggcttctatccgagcgatatcgctgtcgaatgggagagcaatgggcagcctgagaataactataaaacaacgccacccgtcctcgactccgatggctcattcttcctgtacagtaaacttacagtagataagagtagatggcagcagggtaacgtctttagttgctccgtgatgcacgaggcattgcacaatcattacactcaaaaatctctgtccctgagtccgggcaaaggcggttcagatagctggatggaggaggtcataaagctttgtggacgagaactcgttcgcgcccagatagctatttgtgggaaatcaaccgggggtggagaaggtggcggagaagggggaggcgaagggcgccaactgtattctgcattggctaataagtgctgtcacgtaggatgtacaaaaaggtctctggcgagattctgc

SEQ ID NO: 384 - DNA, 945 bp, synthetic construct
  source 1..945: mol_type = other DNA
atggagaccgacaccctcttgttgtgggttctcctcttgtgggtgcccggcagtactggagacaagacgcacacttgtccaccttgccctgcgccggaagctgctggaggccccagtgtctttttgttcccgcccaaaccgaaggacactttgatgataagtcgcacgcccgaggttacctgtgtggttgtcgatgtctcacacgaagatccggaggtgaagtttaattggtatgtagatggcgtggaggttcataacgccaaaacgaaacccagagaagaacaatataacagtacatatcgagtagtatccgttctcactgtcctgcatcaagactggttgaacgggaaggaatataagtgcaaggtgagcaataaagcactcccggccccaatcgaaaagaccatcagcaaagcgaaggggcaacctcgagaaccccaggtatatacgctcccccctagtcgggatgaacttactaaaaatcaggttagcctcacttgccttgttaaagggttctatcccagtgatattgccgtcgaatgggaatcaaacgggcagccggaaaataactacaagacaacccctcctgtgctcgatagcgatggctcttttttcctctacagcaaacttaccgttgataagagccggtggcaacaaggtaatgttttctcctgctccgttatgcatgaagcactccataaccattatacccaaaaaagcctgtcacttagtccgggtaaaggaggtagtgattcttggcaggaggaggtaatcaaactttgtgggagggagctggtacgagctcagattgctatatgtggaaaaagcacgggcggaggagaaggaggtggcgaaggcgggggtgaaggtcggcaactctactccgctctcgctaataagtgctgccacgtcgggtgtacgaagcgctccctggcgcgattctgc

SEQ ID NO: 385 - DNA, 945 bp, synthetic construct
  source 1..945: mol_type = other DNA
atggaaacagataccctcctcctctgggtccttcttctttgggtgcctggctcaactggagataaaacgcacacgtgtccgccctgcccagcgcctgaagccgcaggcgggccgtccgtcttcctctttcctccaaaacccaaagacacacttatgatcagtaggaccccagaggtaacctgcgtcgtggtcgacgtttcccatgaagacccagaggtcaagttcaactggtacgtcgacggtgtcgaagtacataatgctaaaacgaagcctcgggaagagcagtacaactctacctaccgcgtcgtttccgtactcaccgtacttcaccaggactggcttaacggtaaagagtataaatgcaaagtatctaataaggctctcgccgcgccgattgagaagacaatttcaaaggccaaggggcagccgcgggagccccaagtgtataccttgcccccgtcccgagatgagctgactaaaaaccaagtaagcttgacttgcttggtcaaaggcttctacccttccgatatagctgtcgaatgggagtcaaatggccaaccagagaacaattataaaactacacccccggtcttggattctgatggctcattttttctctattctaaactgaccgtggataagtctcgctggcagcaaggtaacgtgttcagttgctctgttcttcacgaagcactgcacagtcattacactcagaagagtcttagcctgagccctggtaaagggggttctgattcctggcaggaggaagtaataaaactctgtggccgggagttggtacgggcgcagattgcgatatgcggtaagagcaccggcggaggcgaaggcggtggggaaggaggaggagaagggagacaactctattccgcattggcaaataagtgctgccacgtcgggtgtaccaaacgatcccttgcacggttctgt

SEQ ID NO: 386 - DNA, 948 bp, synthetic construct
  source 1..948: mol_type = other DNA
atggagacggacaccctccttctctgggttttgctcctttgggtccctggttccactggagataagacccatacgtgccccccttgccctgcgcctgaggcagcgggtggcccatcagtctttttgttcccgcccaagccaaaggacaccctcatgattagtagaacaccggaggttacgtgcgtcgtagtggatgtcagccacgaggatcccgaggttaagtttaactggtacgttgatggggttgaggtccataatgcgaagactaagccgagagaggaacagtacaattccacgtatagagttgtctctgtactgactgtgctgcatcaagattggcttaacggtaaggagtacaagtgcaaagtctctaataaggctcttcctgcacccattgagaaaactataagcaaagcaaaaggtcaacctcgcgaacctcaggtgtacacactgccaccctctagggacgagcttaccaaaaatcaagtatctcttacctgccttgtgaaagggttttatccctcagatattgcggttgagtgggagtctaacggacaacctgagaacaactataagactactcccccggtgcttgattcagacgggagtttttttttgtatagcaaacttaccgtcgacaaaagccggtggcaacagggcaatgtattcagttgttctgtaatgcatgaagctttgcataatcattacacccaaaagagtctttccctgtctcctggaaaaggggggtcagactcctggatggaggaggtgatcaaactgtgtgggagagagctcgtccgggctcagatagctatatgcggcaagtctacgggtgggggagagggcggaggagagggcggtggagaaggaggcggccaactctacagcgctctggccaataaatgttgtcatgtcgggtgtactaagcgctcactggcacgcttttgc

SEQ ID NO: 387 - DNA, 948 bp, synthetic construct
  source 1..948: mol_type = other DNA
atggaaaccgacacccttttgttgtgggtattgctgttgtgggttcccggtagcacgggggacaagacgcatacatgcccgccatgcccggcccccgaagctgctgggggaccatccgtattcctcttccctcccaaaccaaaagacacgttgatgataagtagaacaccagaggtaacgtgcgtggttgtcgatgtttcccacgaagatccggaggtaaaattcaattggtatgtagatggggtggaagtgcacaatgccaaaacaaagccgcgagaagaacaatacaatagtacttaccgggttgtgagcgtgctcacggtgttgcaccaagactggctcaacggcaaggaatacaagtgcaaagtatctaataaagctctgcctgcgccgatagagaagaccatcagtaaggccaaagggcagccccgagagccgcaagtttacactcttcctccgagcagagatgaattgaccaagaaccaagtaagtttgacgtgcctggtgaagggcttctacccctcagacattgcggtggagtgggaaagtaatggtcaaccggaaaacaactacaagaccacgccgcccgtcctcgactccgatgggtctttctttctttattcaaagttgacagtagataagtcaaggtggcagcaaggtaacgtgtttagttgtagtgtaatgcacgaggccctgcataatcattatacccaaaagagtttgagcctctcaccaggaaaaggcggatcagacagctggcaggaggaggtaattaaattgtgtggacgggagttggtcagggcgcaaatagccatctgcggtaagagcacgggtggaggagagggtggaggggaaggtgggggagaaggcggcgggcagctctattctgcactcgccaacaagtgttgtcacgtcggatgcacaaagagatctcttgctcgattctgc

SEQ ID NO: 388 - DNA, 948 bp, synthetic construct
  source 1..948: mol_type = other DNA
atggagactgatactcttttgttgtgggtactgctcctgtgggttccaggctccacaggagacaaaacacacacctgtccgccttgcccggctcctgaagccgcgggtggccctagtgtgtttttgtttccgccgaaacctaaggataccctcatgataagccggacgcccgaggttacctgtgtcgtggtcgatgttagtcatgaggatccagaagtcaagtttaattggtacgtcgacggcgttgaagtccacaatgcaaaaactaaaccgcgagaagaacagtacaactccacctacagagttgtctcagttttgacagttctccatcaggattggctcaatggaaaggaatataagtgcaaggtcagcaataaagcgcttgccgcccctatagagaagaccattagcaaggcgaaaggacagccccgcgagccccaggtctatacgctgcctcctagcagagatgagctcacgaaaaatcaggtcagcttgacatgcttggtgaagggcttctaccccagtgacatcgcagttgaatgggagagcaacggccaacctgagaacaactacaaaacaacgcccccggttcttgacagcgatgggtccttctttctttactctaagcttacagttgataaaagcaggtggcagcaggggaatgtgttctcatgttccgtactgcatgaggctctgcattctcactacacccaaaaaagccttagcctgagccccggtaagggaggtagtgactcatggcaagaggaagtgattaagctctgcggccgggagttggtgagagcccaaatcgccatttgcggtaaaagtaccggagggggcgagggaggaggcgaaggtggaggtgaaggaggtggacagttgtactcagctcttgcaaataaatgttgtcatgttggttgcacgaaaagatctcttgcgaggttctgt

SEQ ID NO: 389 - DNA, 945 bp, synthetic construct
  source 1..945: mol_type = other DNA
atggagactgacactcttttgttgtgggtgcttcttctgtgggtacctggatccactggggataagacgcatacttgtccaccgtgccccgcaccggaagcggctggtggtccatcagtttttctgttcccaccgaaacctaaggacacgttgatgatatcacggacaccagaggttacgtgcgtagtggtggatgtgagccacgaggatccagaagttaaatttaattggtacgtagatggagtggaggttcataatgcgaagacaaagcctcgcgaggaacagtataattccacctatcgcgtcgtatctgtgcttacggtacttcaccaagactggttgaacggtaaggaatataaatgcaaggtttccaataaagcacttcctgcgccaattgagaagacaatatccaaagctaaaggtcaacccagggaaccgcaagtctacactctccccccgtctcgcgatgaattgacgaagaaccaggttagtctcacctgcctggtcaaggggttttacccctctgacatagctgtagaatgggagtctaatggacagccagagaacaattacaaaacgacccccccggtcctcgattctgatgggagtttttttctttattcaaaattgactgtcgataagtcaagatggcaacagggtaacgtattttcttgcagtgttatgcatgaagcattgcacaaccactatacacaaaaatcattgagtttgagtcccggtaaagggggaagcgactcatggatggaagaagtaatcaagctgtgcgggcgagagcttgtgcgagctcagatagcaatctgtggtaagtctacaggtggagagggtggcggtgaagaaggcgggggagagggaggccagctttattctgccctggctaacaagtgctgtcacgttggatgcacgaagcgctccctggcccgattctgc

SEQ ID NO: 390 - DNA, 945 bp, synthetic construct
  source 1..945: mol_type = other DNA
atggaaaccgatacattgcttttgtgggtcctccttctttgggttcctggctctacaggcgataagacgcatacttgtcccccatgtcccgctccggaagccgctggcggcccctccgtttttctgttcccgccgaaaccgaaagacaccctgatgatatcacgcactcccgaggtcacttgcgtggtagtcgatgttagtcatgaagatcctgaggtcaaattcaattggtatgtagatggcgttgaggtacacaacgcgaagacaaaaccccgagaagaacagtataactcaacctaccgcgtagtttcagttcttaccgtactgcaccaagactggttgaacggtaaagagtacaaatgtaaagtcagcaataaagctttgccagcacctatcgaaaaaaccatcagtaaggccaagggtcaacccagggagccgcaagtgtacactcttccccctagcagggatgaattgaccaagaatcaggtctctttgacgtgcctcgttaagggtttctatcccagcgatatagccgtagaatgggagtctaacggtcagccagaaaataactataagacaaccccgcctgttttggattccgacggctctttttttctctactctaagttgaccgttgataagagcagatggcagcagggaaacgtattttcttgttccgtgatgcacgaagccctgcacaatcactatacgcaaaagtctctgagcttgagtccgggtaaaggcggttctgactcctggcaggaggaagtcataaaactctgcggaagagagctcgtaagggcgcaaatcgctatttgtggtaagagcaccggtggggaaggaggcggtgaagagggtggcggcgagggtgggcaattgtattccgcgcttgccaataaatgttgtcacgtaggctgcacaaagcgaagtctcgctaggttctgc

SEQ ID NO: 391 - DNA, 942 bp, synthetic construct
  source 1..942: mol_type = other DNA
atggaaaccgacaccttgcttttgtgggtgctcttgctgtgggttccggggagcactggcgacaagacccacacatgtcccccgtgtccggcaccagaagcagcggggggaccgtcagtattcttgtttccaccgaagcccaaagacacattgatgatttcacgaactcctgaagttacctgtgtggttgtagatgtatcacacgaagacccagaagtcaaattcaattggtatgtcgacggggttgaagttcacaatgcgaagacgaagccccgggaggaacagtacaacagcacgtacagggttgtgagcgttcttactgtattgcaccaggattggctcaacggcaaggagtataaatgtaaagtttctaataaggctcttcctgccccaattgaaaagacgatatctaaagcgaagggccaaccacgggaacctcaggtgtacacacttccgcctagcagggatgagttgaccaagaatcaagtctctttgacgtgcctggtcaaggggttttacccatcagatatcgccgtcgaatgggagtcaaacggacaacccgaaaataactataaaactactccaccagttctggatagcgacggctcattttttctgtattcaaagctcactgtagacaagtctaggtggcagcagggtaatgtcttctcctgctcagtaatgcatgaggctcttcacaaccactatactcaaaagagcctttccctgtcacctggcggtggaagcgactcatggatggaggaggtaataaagctctgcggaagagaactggtacgcgcacaaatcgcaatttgtggtaagagtactggcggggaaggaggtggggaagaagggggcggtgagggcggacagctctattctgcacttgcaaacaaatgttgccacgtgggatgtactaagcgaagccttgcaagattctgc

SEQ ID NO: 392 - DNA, 942 bp, synthetic construct
  source 1..942: mol_type = other DNA
atggagaccgacacactgttgctgtgggtactcctcctgtgggtgccaggaagcacgggcgataaaacccacacatgccctccatgccctgctccagaggccgccggtgggccatcagttttcttgtttccgcctaaaccaaaggacacgcttatgatctccaggacccccgaagttacgtgtgtggtggttgatgttagtcacgaggacccggaagtcaagttcaactggtacgttgatggtgtagaggtgcacaatgcaaagacgaagccacgcgaagaacaatacaacagcacatatcgagttgtgagcgtactcacggtactgcatcaggactggctgaacggtaaagaatacaaatgtaaagtctccaataaggcacttcctgcgccgatagaaaaaacgatcagtaaggccaagggccaaccccgagaaccacaggtatatacgctcccaccgtcacgagacgagttgacaaaaaatcaggtctccctgacttgcctcgtgaaaggtttttatccctcagatattgctgttgagtgggaaagcaatgggcagccagagaataattataagacgactcctccggttttggattccgacggtagttttttcttgtatagtaagcttactgtagacaagtcaagatggcaacaaggtaatgtgttctcttgctcagttatgcatgaagctcttcataaccattacacgcaaaagagtctcagtctgagccccggtggcggtagcgacagttggcaggaagaggtgattaagttgtgcggtcgcgagctcgttcgggcccaaattgcaatctgcggaaaatctacgggcggagagggcgggggtgaggagggtgggggtgaaggtgggcagctctatagcgcccttgcgaataaatgttgtcacgtcggatgcacaaagaggtccctcgccaggttctgc

SEQ ID NO: 393 - DNA, 945 bp, synthetic construct
  source 1..945: mol_type = other DNA
atggaaactgacacactgttgctgtgggtgctgctcctttgggtacccggatcaaccggggataagacccacacttgccccccttgccctgcccccgaagcggccggaggtccttcagtatttttgtttccaccgaaacccaaagatactttgatgatatcaagaactcctgaagtcacctgcgtggtagttgacgtatctcatgaggatcccgaggtgaagttcaattggtacgtcgatggcgtcgaggttcataacgctaagactaagccgagggaagagcaatataattccacttatagggtggtgtccgtcttgactgttttgcaccaggattggttgaacgggaaagagtacaaatgtaaggtgagtaataaagctttggctgctcccatcgaaaagacaataagcaaggccaaggggcaacctcgggagccgcaggtgtacacccttcctcccagtagagacgaactgacaaaaaaccaggtgtccctgacctgccttgtgaaggggttttacccgagcgacatagcggttgaatgggagagcaacgggcaacccgagaacaactacaaaactacaccgcctgtcctggactccgatggaagcttcttcctctactccaaactgaccgtggacaaaagcagatggcaacaaggaaacgtattctcatgctcagtaatgcacgaagcattgcacaatcactacacccaaaagtccctctcactctcccctggtaagggcggatcagactcatggcaagaggaggtaattaagttgtgcgggagggagctcgtccgcgcgcaaatagccatttgtggcaagtccactggaggaggcgagggtggaggagagggtggtggggagggcaggcaactctacagtgcgctcgccaataaatgctgccatgttgggtgcacgaagcgcagtctcgcacaattctgc

SEQ ID NO: 394 - DNA, 945 bp, synthetic construct
  source 1..945: mol_type = other DNA
atggagaccgacactctgctgctctgggtactcttgctgtgggtgcctgggtctactggggataagacccacacgtgtcctccatgtccggcaccggaggctgctggcgggccttctgtattcctcttcccacccaagccaaaagacacattgatgatatcaaggacgccggaagtcacctgtgttgttgtggacgtttcccatgaagacccagaggtaaaattcaattggtatgtggacggcgtagaggttcacaacgccaaaaccaaaccccgagaggaacagtataatagcacatatcgagtagtatctgttctcacagtgctccatcaagactggcttaatggtaaagagtataaatgcaaagtttccaataaagccctcgctgcaccgatcgagaagacaatcagtaaagcgaagggccagcctcgggaaccgcaggtgtatactcttccaccctcaagagacgagctcactaaaaaccaagtttcattgacatgcctcgtcaaaggtttctacccatcagacatcgcggtcgaatgggaaagtaatgggcagccggaaaacaactataaaacgacgccgcccgtcttggattctgatggttcattttttctttactctaaattgaccgtcgataaaagtaggtggcaacaaggaaatgttttttcctgctccgtcctgcatgaagcgttgcacagtcactatacccagaagagtctttctttgtcacccggaaaaggcggttcagattcatggcaggaagaagtaattaaactctgtggccgcgagcttgttagggcgcagatagccatatgtggtaaaagcaccggaggaggtgaaggcggaggcgaaggaggtggggaaggaagacaattgtattctgcacttgcaaataaatgctgtcatgtggggtgcacgaaacgcagtcttgcacaattttgt

SEQ ID NO: 395 - DNA, 942 bp, synthetic construct
  source 1..942: mol_type = other DNA
atggaaaccgatacgctgcttctttgggttcttctcctctgggttccagggtccaccggcgacaaaacccatacctgccccccttgccctgcaccagaagcggcgggaggacctagcgtttttctttttcctccgaaaccgaaagataccctcatgatatcaagaacacctgaggttacttgcgttgtcgtggacgtgagtcacgaagaccccgaggtgaagttcaactggtatgtagatggagtggaggtccataatgcaaaaacgaaaccgagagaagaacaatacaactctacatatcgagtcgtgtcagtactcacggttttgcatcaagattggctgaacggtaaggagtacaagtgtaaggttagcaacaaggctctcgcggcgccgatagaaaagactataagtaaagcaaaaggccagcccagagaacctcaagtttacactctgcctcccagcagagatgaactgactaaaaatcaggtttcattgacctgtctcgtcaagggtttttatccaagcgacatagcagttgaatgggaaagcaacggtcaaccagaaaataattacaaaaccactccaccagtcttggactctgacggatccttctttctctattcaaaattgacggtggataaatctaggtggcagcaaggcaacgtcttctcttgtagcgttatgcatgaggcgctgcacaaccactacacacaaaagtctcttagtttgagcccgggcggcggaagcgactcttggcaagaggaagtgataaaactctgtggtcgagaattggtacgcgcgcagatcgctatctgcggcaagtccacagggggaggggaaggtggcggggaaggtggtggcgagggcaggcagttgtatagtgcacttgccaacaagtgctgccatgtggggtgcaccaagcgcagtttggcacggttctgc

SEQ ID NO: 396 - DNA, 942 bp, synthetic construct
  source 1..942: mol_type = other DNA
atggaaacggacacccttctgctctgggtactgctgctctgggttcctggttctaccggtgataaaactcacacttgtcccccgtgtccggcaccagaagccgcaggagggccatctgtctttctttttcccccaaaacccaaggatacactgatgatctcccgcactcccgaagttacttgtgtcgtagtagacgtttctcacgaggacccagaggtgaaattcaattggtatgttgacggagtagaggtgcataatgccaagacaaagccccgagaggaacaatacaattcaacctacagagtagtgtccgttcttacggttctccatcaggattggctcaacggtaaggaatataagtgcaaggtaagcaacaaagcgctggccgcacccattgagaaaaccatttcaaaagctaaaggccaaccccgcgaaccacaagtttatactctccccccaagtcgcgatgaacttacaaaaaatcaagtctcattgacgtgcttggtcaaaggcttctacccgagcgatatcgctgttgaatgggagtctaatggacaaccggaaaataactataaaactacacccccagtcctcgattcagacggcagcttcttcctgtattcaaaactgacggttgacaaatcacgctggcaacagggtaacgttttttcctgtagcgttcttcatgaagccttgcacagtcactacacccagaagtcccttagcttgtcacctggcgggggttcagactcttggcaggaggaggtaatcaaactgtgcggaagagaactggtgagggctcagattgcaatttgtgggaagagcacgggtggcggtgaaggaggtggcgagggcggaggagaggggaggcaactctacagtgcgttggctaataaatgctgtcacgtcggctgtactaagagaagcctcgccagattttgc

SEQ ID NO: 397 - DNA, 942 bp, synthetic construct
  source 1..942: mol_type = other DNA
atggaaacagatactttgttgctgtgggtactcctcctctgggtacctgggagcaccggggacaagacgcatacttgccctccgtgccctgcaccagaagccgctggtggcccatctgtgtttttgttcccccctaagccaaaagacacattgatgatttcacgaactccagaagtgacttgcgtagttgttgacgtatcacacgaagaccccgaggttaaatttaattggtatgtggacggggtcgaggtgcataacgccaaaaccaaaccccgggaggaacaatataactctacgtatcgggtcgtatctgtgttgaccgtccttcaccaagattggttgaacggcaaggaatataagtgtaaagtgtctaataaagcattggctgccccgatagaaaagacgatctctaaagccaagggccaacccagagagcctcaagtatatactctcccaccgagtcgagatgagctcactaagaaccaggtgtcactcacgtgtctggttaaaggattttaccctagtgatatagccgtcgagtgggaatcaaatgggcagccggagaataactataagaccacgcctccagttctcgattccgatggtagctttttcctttactctaaacttacggtcgacaagtccaggtggcaacagggcaatgtattttcttgctccgtcatgcacgaggctttgcacaaccattacacgcaaaagtcactgtccctgtctcctggaggcggttctgacagttggcaggaggaggtaatcaaattgtgtgggcgggagttggttagggcgcaaattgctatttgcggcaaaagtactgggggcggtgaaggcggaggcgagggaggaggagaaggtcgacaactgtattctgccttggcgaacaaatgctgtcacgtcggctgtacgaaacggtctttggcccagttttgt

SEQ ID NO: 398 - DNA, 942 bp, synthetic construct
  source 1..942: mol_type = other DNA
atggaaactgacactcttctgttgtgggtccttctgctgtgggttcctggctctactggagataagacacacacttgtccgccatgccctgcgccggaagcggcgggaggaccgtccgttttcctgttccctcccaaacccaaagacacgttgatgattagtcgcacgccagaagttacgtgcgttgtcgtagatgtatcccacgaagaccccgaggtgaagttcaattggtatgtagatggggtggaggtccataacgctaagaccaaaccacgcgaggaacaatataattctacgtaccgcgtagtgagcgttctcacagttcttcaccaggattggcttaacggcaaggagtataagtgtaaggtgtctaataaggccttggctgccccgatcgaaaaaacgataagtaaagcaaagggtcaacctagagaaccccaagtgtacactctcccgccatcacgggatgaattgactaagaaccaagtgtcactcacgtgtcttgtaaagggcttctacccatccgatatagccgttgagtgggaatccaatggtcagccagagaacaactataagacaactccgcccgtacttgatagtgacggttcctttttcctttacagtaaattgacggtagataagtctcgctggcagcaaggaaacgtcttttcttgttcagtgcttcatgaggcgcttcactcacactatactcagaagagtttgagtttgtctccaggtggaggcagcgactcatggcaagaggaagtaatcaaactgtgtggtcgcgaattggtacgagcacagatcgcgatctgcgggaaatcaacaggtggcggcgaaggcggcggggaaggcggcggcgaaggtaggcaactttactcagcccttgcgaacaaatgttgccacgtaggctgtactaagagaagtctcgcccagttttgc

SEQ ID NO: 399 - DNA, 945 bp, synthetic construct
  source 1..945: mol_type = other DNA
atggagacagatacccttctgttgtgggtccttctgctttgggtgccgggaagtacaggcgacaagactcatacctgccccccttgtccagcaccagaagcagctggcgggccaagcgtgttcctgtttccacctaagcccaaagatacgttgatgatcagccgcaccccggaagtaacctgtgtagtagtagatgtgtcccacgaagaccccgaagtaaagtttaattggtacgtcgatggtgtcgaagtacataacgctaaaacgaagccccgagaagagcagtacaacagtacttacagagtagtttctgttcttacagtgctgcatcaggattggctgaacgggaaggagtataaatgtaaagtctcaaacaaggcacttgcggcaccaatagagaagacaatatctaaggccaaagggcagcctagagagccacaagtatatacgctgccccccagcagggacgagctgacaaagaaccaagtgtcactgacctgccttgttaagggcttctatccgagtgatattgctgttgaatgggaaagtaacggacagccggagaacaactataaaactactccacccgtgttggatagtgacggtagcttttttctgtactccaagttgacggtagacaaaagtcggtggcagcaggggaacgtattttcttgttctgtcatgcacgaagctcttcacaatcactatacgcagaagtccctctctctctctcctgggaagggtggttccgacagctggcaggaggaggtcattaaactgtgtggtagagagctggtacgggctcaaattgcaatttgtggtaagagtactggcggtggcgaggaagggggtggggaggagggcggaggtaggcagctctactctgctctcgccaacaagtgttgtcacgtcgggtgtactaaaagatcacttgcccgcttttgt

SEQ ID NO: 400 - DNA, 945 bp, synthetic construct
  source 1..945: mol_type = other DNA
atggaaaccgataccctgctcttgtgggtcctcctgctttgggtcccaggttccacaggcgacaaaacacatacatgcccgccgtgtccggcgcctgaagcagcaggaggccccagtgtattccttttccctccaaagccaaaagatacgttgatgatatctaggacacctgaggttacctgcgtcgtagtggacgtatcccacgaagacccagaagtcaagtttaactggtatgtggacggagtggaggtacacaatgcaaagacaaagccgcgagaggaacaatataattccacctatagagtcgtgtcagtccttacggtcttgcaccaggactggctcaatggtaaggagtataagtgcaaagtatcaaacaaagctctcgcagcgcccatcgaaaagaccatcagcaaagctaagggccagccaagagagcctcaagtgtacacgttgccgccttcaagggacgagctcactaaaaatcaggtatcacttacgtgtcttgtcaaagggttttatccttccgacatcgcggttgaatgggagagcaatggacagccggagaataattataaaacgacgccgccggtccttgacagcgatggttcatttttcctttactcaaagctgacggttgataagtctaggtggcagcaggggaacgtcttttcctgtagtgtacttcatgaggcgctccattctcattacactcagaagtcactgagcctttcacccggcaaaggtggatcagactcctggcaagaagaggtaatcaaactctgtgggagggaactcgttcgagcccagattgcaatctgtgggaaaagcacaggcggaggggaagaagggggtggcgaagaaggtgggggcaggcagctctattcagctcttgccaacaaatgctgtcatgtaggctgcacaaagcgatcactggcgagattctgt

SEQ ID NO: 401 - DNA, 945 bp, synthetic construct
  source 1..945: mol_type = other DNA
atggaaaccgacaccctgctgctctgggttcttctcctctgggttcccggctcaaccggagataaaactcatacttgcccaccctgcccggctcccgaggcagcaggtggaccctcagtatttttgttccctccgaaacctaaagatacacttatgattagccggacccctgaggtaacgtgtgtggtggttgacgtaagtcatgaagatccagaagtaaagtttaactggtacgtagacggtgtggaggtacataatgcgaagacaaaaccacgagaggaacagtataactctacctaccgcgtagtaagcgtacttactgtgctccaccaagactggcttaacgggaaagagtataagtgtaaagtcagtaataaagcactggccgccccgatcgaaaaaacaatcagcaaggccaaaggacaaccaagggagcctcaggtctatactcttcccccgagtagggatgagcttaccaagaaccaggtgtctctgacatgccttgtcaagggattttacccgagtgacatagccgtagaatgggagtcaaacggccaacctgaaaacaactataagaccacgcctcccgtactcgactcagatggaagctttttcctctatagcaagctgaccgtcgacaaaagtaggtggcaacagggaaacgtctttagttgttccgtcatgcacgaagctttgcataaccattacacccagaagagtctttccctttcccctggcaaggggggctccgactcctggcaagaggaagtaatcaaactgtgtgggcgcgagcttgtccgcgcgcaaatagccatttgcggaaaaagtactggaggaggagaggaaggcggcggcgaggaaggtgggggcaggcagctgtacagtgccttggctaacaagtgctgccatgtcggctgtacgaaaaggtctcttgctcaattctgt

SEQ ID NO: 402 - DNA, 945 bp, synthetic construct
  source 1..945: mol_type = other DNA
atggaaactgatactcttctcctttgggtgctcctcctctgggttcccgggtccacaggcgataagacacatacctgtccaccctgcccagcacctgaagctgcaggcggccccagcgtattcctgtttcctccgaagccgaaagacacacttatgatttcccggacgcctgaggtaacttgcgtcgtagtagatgtgtctcacgaagaccccgaggtgaaattcaactggtacgttgatggtgtggaagttcataatgcgaaaactaaaccacgagaggagcaatataactcaacttatagagttgtgagcgtcttgacggtactgcaccaggactggctgaatggcaaagagtacaaatgcaaagtctcaaataaggcgttggcggctcccatagagaaaactatcagcaaagccaagggtcaacctcgggagccacaagtgtatactcttccgcctagtcgcgacgagctcacaaagaatcaggtgagtcttacttgtttggttaagggtttctaccccagtgacattgcggtcgagtgggaaagtaacggacagcctgaaaacaactataaaacaacgcctccagtactcgattcagatggttcattctttctttattccaaactcacagtcgacaagagtagatggcaacaagggaacgtgtttagctgtagcgtactccatgaggcactccactctcactatacccaaaagtctctcagcttgtcacccggaaaaggcggttctgacagttggcaagaggaagtgattaaattgtgtgggcgggaacttgtgagggctcaaatcgcgatttgcggcaagtccactggtggcggcgaggaaggaggaggtgaagaaggaggaggtaggcaactgtattcagcgttggcgaataaatgctgccatgttggatgtactaaacggagccttgctcagttctgc

SEQ ID NO: 403 - DNA, 942 bp, synthetic construct
  source 1..942: mol_type = other DNA
atggaaactgacaccttgttgctttgggtattgcttctgtgggttccgggtagcacgggtgataaaacgcatacttgccctccttgcccggcacctgaagctgccggaggtccttccgtgttcctgttcccacctaagccaaaagacacacttatgatttctcgcacaccagaagtaacgtgcgtcgtagttgacgtctcccatgaagacccggaggtaaaatttaattggtacgtcgacggggtagaagttcataacgcaaagactaaaccacgagaagagcaatacaactctacatacagagtagtaagcgttctcaccgttcttcatcaagattggctcaacggaaaggagtataagtgtaaggtgtccaataaagcgttggccgcaccaatcgaaaagaccataagcaaagccaaaggccaaccccgcgaaccgcaggtgtacacacttcccccgtccagggatgaattgacaaaaaaccaagtttccctcacgtgtctcgtcaagggattctacccgagtgatatcgcagttgaatgggaaagcaatggtcagcccgagaataactacaagactactccccctgtgttggactcagacggctcattcttcctctacagtaagttgactgtggacaaaagtcggtggcagcaaggcaatgtcttcagttgtagtgtaatgcatgaagcactccacaatcattacacccaaaaatccctgagcctgtccccgggcggaggttcagattcatggcaggaggaagttataaaactgtgcgggcgcgagttggtgagggcgcagatcgcaatctgtggaaagagtacgggaggtggcgaagagggtggtggagaagagggaggaggtcgacaactgtattccgcgctcgcgaacaagtgttgccacgttggctgcaccaaacgaagcctggctcgattttgc

SEQ ID NO: 404 - DNA, 942 bp, synthetic construct
  source 1..942: mol_type = other DNA
atggagactgacacccttctcctctgggtcctcttgctttgggtccctggctctactggtgacaagacacacacttgtccaccttgcccggctcccgaggcggcaggaggaccaagcgtttttctgttccctcccaaaccaaaggatacgcttatgatctctcgaacgccggaagttacttgcgtagtagttgatgtctcccatgaagatcccgaagtgaagttcaactggtatgtagatggtgtggaagttcataacgcgaaaaccaaaccacgcgaagaacagtataacagtacttatcgggttgtttcagtactcacggtgctccatcaagactggcttaatggaaaggagtataaatgtaaggtaagtaacaaggcattggcggctcccatcgagaagacaatctccaaagcaaaagggcaaccacgggagcctcaggtgtatacgttgccgcccagcagagatgaacttactaagaatcaggtgagtctcacttgtctcgtcaagggcttctatcccagcgatatagccgtagaatgggagagtaacggtcagccggagaacaactacaaaacaaccccgcctgttttggactccgatgggagtttttttctctacagcaaactcacggtagacaaaagcaggtggcagcagggcaatgttttcagttgctctgttctccacgaagccctccactcccactatactcagaagtctctgagtctctcaccagggggaggtagcgatagctggcaggaggaagtgatcaagttgtgcgggcgcgaactcgtgcgggcacaaattgctatatgcggtaaaagtacgggaggtggagaggagggtggaggtgaagaaggcggtggtagacaattgtatagtgcgctcgccaacaagtgttgtcatgtcgggtgtacgaaacggtccttggcgcggttttgc

SEQ ID NO: 405 - DNA, 942 bp, synthetic construct
  source 1..942: mol_type = other DNA
atggaaactgacacacttcttctgtgggtactcttgttgtgggttccgggctcaacgggtgacaagacacatacttgtccaccatgtcccgccccagaagctgcgggaggaccatcagtttttttgttccccccgaaaccgaaggataccctcatgataagtcgaacgcccgaagtcacttgcgtggtggttgatgttagccacgaggacccagaagtgaagttcaactggtacgtggacggggtcgaagttcataatgcgaaaacaaagcctcgcgaggaacagtacaactctacatacagggttgtgtctgttttgacagtcttgcaccaagattggctcaacgggaaggaatataagtgtaaggtaagcaataaagcactggcggccccgatcgaaaaaacgatatccaaggccaagggccagccccgagagcctcaggtatatactctgccgccaagccgggatgaactgactaaaaaccaggtctctttgacttgtcttgtcaagggattttacccaagtgacattgcggtagagtgggaaagcaacggtcaaccagaaaacaattacaagacgacaccgccggtactcgactcagatggatcctttttcctgtatagcaagctgacagtggacaagtcccggtggcagcaagggaacgtattttcatgcagcgtgatgcatgaggctcttcacaaccattacacacagaaaagtctgtcattgagccctggcggcgggagcgattcttggcaagaagaagttataaaactttgcggtcgagagctggttcgggcacaaattgctatctgcggaaaatctacaggaggaggcgaggagggagggggcgaagaaggcggggggagacagttgtacagtgcgctcgctaacaagtgttgccacgtcggttgcacaaagagatccctggctcaattctgt

SEQ ID NO: 406 - DNA, 942 bp, synthetic construct
  source 1..942: mol_type = other DNA
atggagacagatactctcttgctgtgggtgctgctcttgtgggttcctggaagtaccggtgataaaactcacacctgtcccccgtgtcccgcaccagaagcggccggtggtccctccgtttttctcttccctcctaaacctaaggacacacttatgattagcagaactccagaagttacgtgcgtagtcgttgacgttagtcatgaagatcctgaggttaagttcaactggtacgtagacggagtagaggtccacaacgccaagacgaaaccccgagaagagcagtataattctacctatcgagttgtttcagtattgacggtgcttcaccaagattggctgaatggcaaagagtataagtgcaaggtaagcaacaaagcactcgcggctcctatcgagaaaactatttccaaagctaagggccagcctcgcgaaccacaagtctataccctgccaccgagtcgggacgaactcaccaagaaccaagtgtctcttacttgcctcgttaaaggtttttatcccagcgacatagccgtcgaatgggagtccaatggccaacctgagaacaactataaaactacccctcctgtacttgatagcgacggaagttttttcctctattcaaaactcacagttgataagtctcgatggcaacagggcaacgtcttctcttgcagtgtgttgcatgaagctctgcactctcattacacacagaagagtttgtctctcagtccaggtggcggctcagatagctggcaggaagaagtaatcaagttgtgcggcagggaactggtaagggcacagatagccatttgtggaaaatctacgggtggcggtgaggaaggcggcggagaagaagggggaggtcggcagctgtatagtgcactcgcaaacaagtgctgccatgtcgggtgcaccaagcgatcccttgcccagttttgc

SEQ ID NO: 407 - DNA, 945 bp, synthetic construct
  source 1..945: mol_type = other DNA
atggagacggacactcttctcctgtgggttctcctcttgtgggttccaggatctaccggcgataagacgcacacatgcccaccctgtcctgcgcctgaagccgcggggggacccagcgtttttctcttcccgccgaaaccgaaagacacacttatgatcagccggactcccgaggttacctgcgtggtggtagatgtatctcacgaggatcccgaggtcaaattcaactggtacgttgatggggttgaagttcataatgccaaaacgaagccaagagaagagcagtataactccacatatagagttgtttccgtcttgactgttcttcaccaagattggctgaatgggaaggagtacaaatgtaaagttagcaacaaggcactcgccgctcccattgaaaaaactataagcaaagctaagggccaaccgcgcgaaccacaggtctacacgttgccgccctctagggacgaactcacgaagaatcaggtttcccttacctgcctcgttaaaggattctacccctctgacatagcggttgaatgggagagcaacggtcagcctgagaacaactacaaaacgacgcctccggtgttggattccgacggtagttttttcctctatagtaagctgacagtggataaatctcggtggcagcaagggaatgtattctcctgttcagtcctgcatgaagccctccactcccattatacacagaaatctctttctctgagtcccggtaaaggtgggagtgactcttggcaggaagaggtaattaagttgtgtggaagggagctggtaagagcacagattgccatctgtggcaaatccacgggcggcgaaggtgaggggggtgagggggaaggggggtccagacaactgtattctgctctggcgaataagtgttgccatgtagggtgcactaaacggtccttggcgcagttctgt

SEQ ID NO: 408 - DNA, 942 bp, synthetic construct
  source 1..942: mol_type = other DNA
atggagactgacacactgctcctctgggtccttttgctctgggttccggggtccaccggtgataaaactcatacgtgcccaccttgccccgcaccggaggctgctggaggaccctctgtcttcctgttcccgccgaagcctaaagacacattgatgatcagtcgaacaccggaagtcacctgtgtagtggttgatgtgagccatgaggaccctgaagtaaaatttaactggtatgttgatggcgtagaagtacacaacgcgaagactaaaccaagggaagagcaatacaactctacctatagggtcgttagcgtactgactgtgcttcaccaagactggcttaacgggaaggagtacaagtgcaaagtgagcaataaggccctcgccgcgcctatcgagaaaaccatttccaaagccaagggtcaaccaagggagcctcaggtttacaccctgcccccttcaagggatgagttgacaaaaaaccaggtaagtctgacgtgtctcgttaagggattctacccgtcagatatcgcggtagagtgggagagcaacggtcagccagaaaataattacaaaacaacacctccagttttggactctgatgggagtttttttctttattctaagttgacagtggataagtcacgctggcaacaggggaacgtatttagctgctcagtacttcatgaagcgttgcattctcactacacacagaagagcctctccttgagtcccggaggtggctctgattcttggcaggaggaggtaataaaactttgtggtagagaactggttcgcgctcagatagctatttgtggaaaatccactggcggtgaaggtgaaggtggagaaggagagggcggaagccggcagttgtactctgccctggctaataagtgctgtcacgtgggctgcactaagcggagcttggcaagattttgc

SEQ ID NO: 409 - DNA, 942 bp, synthetic construct
  source 1..942: mol_type = other DNA
atggaaaccgacacgctgctgctgtgggtgctgttgttgtgggttccaggctcaactggcgataaaactcatacctgtccaccttgtcctgcgcctgaggcagctggagggcctagcgtgttcctgttcccccccaaacccaaagacacgctcatgattagccgaacccctgaagtgacctgcgttgttgtggacgtaagccacgaagaccccgaagttaagtttaattggtacgtcgacggtgttgaggttcataacgcgaagactaagccgagagaggagcaatataacagcacctaccgcgtagtctcagttcttaccgtgctccaccaggactggcttaacgggaaggaatacaaatgcaaagtttccaacaaagccttggcagccccaatagagaagacaatatctaaggcgaaaggccaaccgcgggaaccgcaagtttataccctcccaccgagcagggatgagctgacaaaaaatcaggtttccctcacttgtctggtcaagggattttatccttcagacatagccgttgaatgggagagtaatgggcagccggagaataattacaagaccacccccccggtgttggacagcgacggttccttctttctctattctaaacttaccgtcgacaaatcacggtggcaacaaggaaatgtattctcatgcagtgtattgcacgaagctctgcactctcattacacccaaaaatccctctctctcagccctggcggtggatctgattcttggcaggaagaggtgattaaactgtgtgggcgagagcttgtccgagctcagatcgctatttgtggcaagagtaccggaggcgagggtgagggaggcgaaggcgagggcggaagccggcaactctatagcgcactcgctaataaatgttgtcatgtcggctgcacgaagcgctcactggcgcagttctgc

SEQ ID NO: 410 - DNA, 885 bp, synthetic construct
  source 1..885: mol_type = other DNA
gacaagacccatacatgtccgccttgtcctgcgcctgaggcagcaggcggaccatcagtcttcttgtttccccccaagccgaaggacacccttatgatctcacgcacccccgaagtaacttgtgtagtcgttgatgtctcacacgaagacccggaagtaaagtttaattggtatgtcgatggtgttgaggtccacaacgctaaaacgaaaccgcgggaagaacaatacaactccacatatcgagtagtctccgtcctgactgttcttcaccaggactggctgaatggtaaagaatacaaatgtaaagtgagtaacaaggcccttgcagcacccatcgagaagacgatatccaaagccaaggggcaaccgcgcgagccacaagtttacacgctcccaccctcaagagacgaactcaccaaaaatcaagtgtccctgacatgtctggtgaaaggattctatcccagcgacatagctgtagaatgggagagtaatggccaacccgaaaacaattacaaaactacccccccggttttggatagtgatggttcattcttcctctatagtaaacttaccgtggataagtctcggtggcagcaggggaacgtgtttagctgttcagtcctccatgaggcactccatagtcactatacgcaaaagtcattgtccctttctccgggcaagggcgggtcagactcctggcaggaagaggtaattaagctttgtgggcgagaactcgttagggcacagatagcaatctgcgggaaaagtacagcttccgatgctgccggggctgacgccaatgcgggagcacgccagctctactcagccctcgccaacaagtgttgtcatgtaggttgcaccaaaagaagtctggcacagttttgc

SEQ ID NO: 411 - DNA, 885 bp, synthetic construct
  source 1..885: mol_type = other DNA
gacaagacgcatacttgtcctccctgcccagctcccgaagcggctggggggccctctgtctttctgtttccgcctaagcccaaggacacgctcatgataagtcgcactccggaagtcacctgtgttgtcgtcgatgttagccatgaagatccagaggtgaaatttaactggtacgtcgacggagtggaggttcacaatgctaaaaccaaaccgcgagaagagcaatacaattccacgtatagggtcgtctccgtcctgacagtactccatcaggattggctgaatggaaaagaatacaagtgcaaggtttccaataaagccttggccgcacctattgagaaaacgatatcaaaagctaagggacaacctcgggagccgcaagtatatacactccccccttctagggacgaactgacaaaaaaccaagttagtctgacttgtcttgtgaaaggtttttacccgagtgatatagccgtagaatgggagagcaatggccagcccgaaaacaattacaaaacaactcccccagtattggacagtgacgggtcattttttttgtattctaaattgaccgtagacaagtcacgctggcaacaagggaatgtatttagctgttccgtccttcatgaggcgctccatagccattacactcagaagtctttgtcactgtcaccgggcaagggtggttctgattcatggcaagaggaagtgattaagctgtgcggtcgggagttggtaagagctcaaattgcgatttgtggcaagagcactgcgtccgatgccgcaggtgctaatgccgacgccggtgcgagacagctttattctgcgctggccaacaagtgctgccacgtcggatgcaccaaacggagccttgctcagttttgc

SEQ ID NO: 412 - DNA, 885 bp, synthetic construct
  source 1..885: mol_type = other DNA
gacaaaactcatacttgtccaccatgcccagcccccgaggcggctggcggccccagcgtattccttttccccccaaaacctaaggacacgcttatgatatctagaaccccggaggtcacatgtgtcgtcgtagacgtaagtcacgaagatcctgaagtcaagtttaactggtacgtcgatggagtcgaggtccataatgctaaaacgaagcctcgcgaagaacagtataattctacctatcgcgtagtctctgtcctcaccgtcttgcatcaagactggttgaacggcaaggagtacaagtgtaaggtttcaaacaaagcccttgccgcgccgatagagaaaacaattagcaaagcgaaggggcagccgagagagccgcaagtgtatacccttcctcctagtagagacgagttgaccaaaaaccaggtgtcacttacatgcctcgtgaaaggcttctacccgagtgatattgcagtcgagtgggaatccaacggccagcccgagaataactacaaaacgacgccgcccgtactggacagtgatggaagtttttttttgtactcaaaactcacggttgacaaaagtcggtggcagcaagggaacgtttttagctgctctgtcctccatgaagcactccattctcattatacccagaagtctctgtctctctcccctggtaagggaggttctgacagttggcaggaagaggtaataaaactctgcggtcgagagcttgttcgagcacaaattgctatatgtggaaaatctaccgcttcagacgccgccggagctgatgcggatgccggggctcgccagctctatagcgccttggccaacaaatgttgtcacgttggctgcacgaagcgctccctggctcagttttgc

SEQ ID NO: 413 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatggcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcacgttgggtgtaccaagcggtcccttgcgcaattctgc

SEQ ID NO: 414 - DNA, 885 bp, synthetic construct
  source 1..885: mol_type = other DNA
gataaaactcatacttgccccccctgccccgcgcctgaagctgcaggggggccatcagtcttcttgtttccaccaaaacctaaggatactctcatgattagccggacccctgaggtgacatgtgttgtggtcgatgtatctcatgaagatcccgaagtaaaatttaactggtacgtagacggggttgaagttcataacgcgaaaacgaaacctcgggaggagcaatataatagcacgtatagagttgtttcagtccttacagttctccaccaagactggctgaatggcaaggagtataagtgtaaagtatccaataaagccttggctgcgccaatcgagaagacgatcagcaaagccaaaggtcagcctcgcgaaccgcaggtctatacattgcccccttcacgcgacgaactcacgaaaaatcaagtctctttgacttgccttgtgaaaggcttctacccctccgatattgccgtcgaatgggaaagcaatggacagccggaaaataattacaaaacgacaccccccgtgttggattccgatgggtccttcttcctctattccaagctgacggtcgataagtctcgatggcagcagggaaatgtcttctcttgctccgtccttcatgaggcattgcacagccattatactcaaaagagtctctctctgtctccaggcaaagggggttccgactcttggcaagaagaggtcataaaactgtgcggccgggagctcgtcagagcgcagatcgctatatgtggaaaatccaccgcgagtgacgcagcaggtgcacaagccgacgcaggagctaggcaactgtactcagcccttgccaataagtgttgtcacgtaggttgtactaaacgctccctggcacaattttgt

SEQ ID NO: 415 - DNA, 885 bp, synthetic construct
  source 1..885: mol_type = other DNA
gataagacacacacgtgcccaccctgccctgccccagaggcagccggaggtcctagtgtgtttctgttccccccaaagcccaaggacacccttatgatatctaggacaccagaagttacgtgcgtcgttgtggacgttagccacgaagacccagaagtgaagtttaattggtacgttgatggagtcgaagtgcacaatgcaaaaacaaaaccacgagaagagcagtataacagtacttatagagtagtcagcgtccttactgtattgcatcaggattggctgaatgggaaggaatataaatgtaaggttagcaataaagcccttgcggctcctatcgagaaaactattagcaaagcaaagggccaaccccgagagccccaagtttatacactgccacccagtcgagatgagctgactaaaaatcaagtatccctgacctgcttggttaaggggttttatcctagtgacatcgcggttgagtgggaatccaacggccaaccggagaataattacaaaaccacgccacctgtattggattccgatggtagcttctttctctatagtaaacttacagtcgataagtcaagatggcagcagggaaacgtattttcatgctcagttctgcatgaggccttgcactcccattacactcaaaaatcactgagcctcagtcctggtaagggtggctctgactcatggcaggaggaagtaatcaagctgtgtgggagggaattggtaagggctcagattgcaatttgtggaaagagcacagcgtctgacgctgcaggtgccgacgcacaggcgggcgcgaggcagctctacagtgctcttgcgaacaagtgttgtcatgtaggttgcacgaaacgaagtttggcgcaattctgt

SEQ ID NO: 416 - DNA, 885 bp, synthetic construct
  source 1..885: mol_type = other DNA
gataaaacacacacctgccccccttgcccagcacctgaagcagcgggtggtcccagcgtttttcttttcccccctaagccaaaggacacgctcatgataagtcgcaccccggaggtcacctgcgtcgttgttgacgtatcacatgaagatcctgaggtgaagtttaattggtacgtagatggtgttgaggtccacaacgcaaagacgaaaccgagggaagaacagtacaacagtacttatcgcgtagtctccgttctgactgtcctgcatcaagattggttgaacgggaaggagtacaagtgcaaagttagtaacaaggctctcgcggccccaattgagaagacgatatccaaagcgaaaggacagccgagagagccccaagtctacactctgcccccttccagggatgagctcaccaaaaatcaggtcagtctcacgtgcctggttaagggattctacccaagtgatatagcagttgaatgggagagtaacggccagcccgagaacaactataaaactacaccgcccgttcttgattccgatgggtctttcttcctttatagtaagctcaccgttgataagtcccgatggcagcaaggtaatgtcttctcatgttcagttcttcatgaagccctgcattcccattatacacaaaagagcttgtccttgtcaccgggcaaaggcggtagcgattcttggcaagaagaagttataaagttgtgcggtagggaactggtacgcgctcaaatagctatatgcggtaagtctactgcttcagatgcggctggcgcacaggcacaggccggtgctagacaactctatagtgcgctggccaacaagtgctgccatgtggggtgtacaaaacggagtcttgcccagttttgt

SEQ ID NO: 417 - DNA, 885 bp, synthetic construct
  source 1..885: mol_type = other DNA
gacaaaactcatacatgccccccatgcccagcacccgaagcggccggaggtccgtctgtctttctgtttccgccgaaacctaaagatacgttgatgattagcagaacccctgaggtaacatgtgtggtagtcgatgtctcccatgaggaccccgaggtaaagttcaattggtatgttgacggcgtcgaagtccataacgcaaaaacgaagccccgagaggagcaatataactctacctatcgcgttgtttctgttttgactgtgttgcaccaggattggctcaacggcaaggaatacaaatgtaaagtgtccaacaaggcccttgctgcacctatcgaaaaaacgattagtaaggcaaagggacaaccgcgcgaaccacaggtatatactttgccgcctagcagagatgaactcaccaagaatcaagtttcccttacctgtttggttaaaggattttacccgtctgacatagctgttgaatgggagagcaatggtcagccggaaaataattataaaaccaccccgccagtattggattcagatgggtcctttttcttgtattctaaacttaccgtggataagtctaggtggcaacagggaaacgtcttttcatgtagtgtacttcatgaagccctccatagtcactacacgcagaaatccttgtctcttagtccgggtgaaggtgggtctgattcctggcaggaagaggtgataaagctctgtggtcgggaacttgttagggcgcagatcgctatttgcggcaaatctacagcatcagatgccgccggagctgatgcgaacgcaggagcgaggcagctgtactccgcacttgctaacaagtgttgccatgtcggctgcaccaagaggagtcttgctcaattctgc

SEQ ID NO: 418 - DNA, 885 bp, synthetic construct
  source 1..885: mol_type = other DNA
gataagacccatacatgcccgccatgtcccgcacccgaggcagcgggtggaccctctgtctttctgttccctccaaagccaaaagataccctgatgattagccgaaccccggaggtgacttgtgtcgtagtagatgtcagtcacgaggatcccgaagtaaagtttaattggtatgtggacggtgtggaggtacataacgctaagacgaaaccccgagaggaacaatacaactctacgtacagggtcgtctcagtgctcacggtcctgcaccaggactggcttaatgggaaggaatataaatgcaaagtctctaataaggcgcttgctgcacctattgaaaaaacgatttctaaggcgaagggacaaccccgggagccacaagtctacacccttcctccaagcagagatgagcttacgaaaaatcaagtgtctcttacgtgcctcgtaaagggcttttacccatccgacattgcggtggagtgggaatcaaacgggcagccggaaaataactacaaaacaacgccgcctgtattggattccgacggctctttcttcctttacagcaaactgacagtcgataaatccagatggcaacaagggaacgttttttcatgttccgttctgcatgaagcccttcacagtcattacacccaaaagtcactttcactttcaccgggcgagggggggtcagactcctggcaagaggaagttataaagttgtgcggcagggaactggttagagcgcagatagcgatttgcggaaaatctactgcgagtgatgctgcgggagcgaatgcggacgccggggcccgacagctctattccgcactcgccaataagtgctgccatgttggttgtacgaagagaagtcttgcacaattttgc

SEQ ID NO: 419 - DNA, 885 bp, synthetic construct
  source 1..885: mol_type = other DNA
gataagacacatacatgccccccctgcccggctcccgaagctgccgggggaccgtcagtgtttttgtttccgcccaagccgaaggatactttgatgattagtcggacaccagaagtgacatgtgttgtcgttgacgtgagtcacgaggatcccgaggtcaagttcaactggtacgttgatggggttgaagttcacaacgctaaaacgaaaccccgcgaagagcagtataactccacttaccgggtcgtcagtgtcctgacggtcttgcaccaggactggctgaatggaaaggaatacaagtgtaaagtttccaataaagcactggccgccccgatcgaaaaaacaatttccaaagctaagggacagcccagggaaccgcaagtttatactcttccaccctcccgggatgaactgaccaaaaaccaagtgtctttgacgtgcctcgtaaagggcttctacccgtcagacatagctgtcgaatgggagtctaatggacagccggaaaacaattataagactacaccgccggtgcttgatagtgatggaagtttctttttgtactccaaacttacggtcgataaaagccggtggcagcagggaaacgtattcagttgtagcgttctgcatgaagctcttcattctcactacacccagaagtctctgtctctgagccccggagagggtggatctgattcttggcaggaagaagtgataaagttgtgcggccgggaattggtacgcgcccagatagccatttgcgggaagtctacggcgagtgacgcagcaggtgctgacgcggacgctggtgctagacagctgtattctgccctggctaataagtgttgccacgttggctgcaccaagagatccctggcccaattctgt

SEQ ID NO: 420 - DNA, 885 bp, synthetic construct
  source 1..885: mol_type = other DNA
gacaagacacatacttgtcccccctgcccagctccagaagctgccggagggccgtcagtcttccttttccctccaaaacctaaggatacgcttatgatttctcgaacgccagaggttacgtgtgtagtcgtggacgtttcccacgaggatcctgaggtcaagtttaactggtatgtagacggggttgaggtccataatgccaagacaaagccgcgcgaggaacaatacaacagtacatatagggtggtgagcgtcctcacagtcttgcatcaagattggctcaacggcaaagagtacaaatgtaaggttagcaacaaagccctcgctgctcccatcgaaaagacgatttctaaggcgaagggccaaccacgagaaccgcaagtatatactcttcccccttcacgggacgagctgaccaaaaaccaggtatccttgacttgcctggtcaaaggattttacccctctgatattgcggtcgagtgggagagtaatgggcaaccagaaaataattataaaacgacccccccggtactcgacagtgatgggtcttttttcctgtattctaagcttacggttgataagtctagatggcagcaagggaatgtcttctcatgtagtgttctgcatgaagcacttcattctcactatactcagaaatctctttcccttagtccgggagaaggtgggagcgatagttggcaagaggaggtgataaaactgtgtggtcgggagctggtgagagcccaaatagctatctgcggcaaatcaacagcaagtgatgcggcaggagcggaagcggaggcgggagcgcggcaattgtatagtgcccttgctaataaatgctgtcacgttgggtgtactaaacgatctcttgctcaattctgc

SEQ ID NO: 421 - DNA, 885 bp, synthetic construct
  source 1..885: mol_type = other DNA
gataaaacccatacatgtcctccgtgtcccgctccagaagccgctggcgggccatctgtgtttttgttcccccccaagcctaaggatacgttgatgatcagcaggaccccggaggttacatgcgtagtagttgacgtttctcatgaagacccagaagtaaaatttaactggtatgtcgatggcgtcgaagtacataatgctaaaactaagcccagggaagagcaatacaattcaacgtaccgagttgtgagtgtccttacggtcctgcaccaagactggttgaacggcaaagagtacaaatgcaaagtgtctaacaaggcattggccgcgcctatagaaaagaccattagcaaagcaaaagggcagcctcgggaaccccaggtctacacgctgccaccttcccgagatgaattgacgaaaaaccaggtctctttgacctgcttggttaaaggcttctacccaagcgacattgcagtggagtgggagtctaacgggcaacccgaaaacaactataagacgactccccctgttcttgattctgatgggagtttttttctgtacagtaagttgacagtggataaatcaagatggcagcaaggtaatgtcttctcttgttcagtgcttcacgaagcattgcattctcactacacacaaaagtctttgtccttgtctccaggtgaaggcggtagcgattcatggcaagaagaagtcattaagctgtgtggaagggaactggttagggcccaaattgcgatatgtggaaagtctacggcgagtgatgcggccggtgctcaagcggatgcgggtgctagacagttgtactcagcccttgcgaacaaatgttgtcacgttggctgtacgaaacgcagccttgctcaattctgc

SEQ ID NO: 422 - DNA, 885 bp, synthetic construct
  source 1..885: mol_type = other DNA
gataaaactcacacatgccccccatgcccagcaccggaagctgccggaggaccgtctgtattcctctttccgcccaaaccgaaagacacgttgatgatttctcggactcccgaggtaacttgtgtcgtggtcgacgtctcacacgaggacccggaggtcaaatttaactggtatgtcgatggggtggaggtccataatgctaagacgaagcccagagaagaacagtataactctacttatagagttgtaagcgtgctcactgtattgcaccaggactggctcaacgggaaagaatataagtgtaaggtctcaaacaaagctctcgcagccccgatagagaaaacaatatctaaggccaagggccaaccgcgcgagccgcaggtttatacacttccaccctcccgcgatgagctgaccaagaaccaggtctctctcacctgtctcgtaaagggcttttatccctccgacattgcagtggagtgggaatcaaacggccagccggaaaataattacaagaccactcctcccgtcctcgactccgatgggtcatttttcctgtacagtaagctcaccgttgataagtcaaggtggcagcagggcaacgtgtttagctgtagtgttctgcatgaggcgctccacagtcactacacccagaaaagtctgagcctttccccaggtgagggtggtagcgatagctggcaggaggaagtaattaaactctgcggtagagaattggtaagggcccaaattgccatctgcggaaagagcaccgcatcagatgctgcgggcgcggatgcgcaggctggtgctaggcaactctactctgccctggcgaataaatgttgccacgtcggttgcacgaaacgaagtttggctcaattttgc

SEQ ID NO: 423 - DNA, 885 bp, synthetic construct
  source 1..885: mol_type = other DNA
gacaaaacacatacatgccccccttgcccggctcccgaggccgccggtggtcctagcgtctttcttttccctcccaaacccaaagacacacttatgattagcagaactcccgaggtaacatgtgtggtcgtagacgtaagtcacgaagatcccgaagttaaattcaactggtacgttgatggtgtggaagttcataatgcaaaaaccaaaccgcgagaggaacagtataactctacctaccgcgtggtctcagtgctgactgtcctgcatcaggactggctcaacgggaaggaatataagtgcaaagtgagtaataaggcccttgcagctcccatagaaaagacgatatcaaaggctaaaggacagccgagggagccacaggtgtacactttgcctccgagtagagatgaactcactaaaaaccaagtaagtttgacatgcctggtcaaaggtttttaccccagtgatatagcggttgagtgggagtccaatgggcaaccggagaacaactataagactactccacctgtcctggatagcgatggaagtttttttctttactcaaagctgacggtggataagagtcgatggcagcagggcaatgtgtttagctgttctgtgcttcacgaagcacttcactctcattatacccagaagtcattgagcctttcccctggtgaaggagggtcagattcctggcaggaggaggttataaagctgtgtggccgggaactcgtgcgagctcaaattgcgatctgtggaaaatccaccgctagtgatgcggcgggagcacaagctcaagcgggcgctcgacaactttatagcgctttggctaataagtgctgccatgtgggttgtacaaagcgcagcctcgctcaattttgc

SEQ ID NO: 424 - DNA, 885 bp, synthetic construct
  source 1..885: mol_type = other DNA
gataagacgcacacttgccctccttgcccggcacccgaagccgctggtgggcctagtgtattcctgttccccccgaagccgaaggatactcttatgatttcacgcacgcccgaggttacatgcgtagtagtggacgtatctcacgaagatcccgaagtcaagttcaattggtatgtcgacggagtagaagttcacaacgcaaagacaaaaccgcgggaagagcaatacaactccacgtaccgcgtcgtttctgttcttacggtcttgcaccaggactggctcaatggcaaggagtataagtgcaaggtatccaacaaggcccttgccgcacctattgaaaagactatcagcaaggccaagggacagccaagggagcctcaagtctacacgctcccgcctagtagagacgagttgacaaagaatcaagtgagtttgacttgtctggttaaaggtttttacccgtcagatattgcagtagaatgggaatctaacggacaacccgaaaacaactataaaacgacgcctcctgtgttggattcagatgggtcattttttctctactcaaagctcacggtagataaatcaagatggcaacaaggcaatgtattttcctgctccgtgctccacgaggctctgcacagccattatacgcaaaagagtctgtctttgagcccaggtgagggtggctccgattcctggcaggaggaagtaattaagttgtgcggcagggaacttgttcgcgcacaaatagccatttgtggtcagagcacagcatcagatgccgccggagccgacgccaacgcaggtgcccgccaactttattctgccctcgcaaacaaatgctgccacgtcggctgcacgaagaggagcctcgcccaattttgc

SEQ ID NO: 425 - DNA, 885 bp, synthetic construct
  source 1..885: mol_type = other DNA
gataagacccatacgtgcccgccatgtccagcccccgaggcagccggaggtccttccgttttccttttcccccctaagcccaaggacactctgatgatctcccggacgcctgaagtcacttgcgtagtcgtagacgtttcacatgaggatccagaagttaaatttaactggtacgtcgatggcgtcgaggtccataacgcgaaaaccaagcccagggaggaacaatataactccacctatagggtcgtgagtgtgctcaccgttttgcaccaagactggctcaacgggaaagagtacaaatgtaaagtttcaaataaggctttggccgccccaatagagaagactatatccaaggctaagggacagcctcgagaaccgcaggtatatacgcttcctccgtctagggatgaactcacaaaaaaccaggtttctttgacctgcttggtaaagggattttatccctccgacattgcggtcgaatgggagagcaacggacagccggaaaacaattacaaaacgacacccccggttttggactctgatggaagcttcttcctctatagtaagttgaccgtagacaagtctcgctggcagcagggaaacgtcttcagttgctcagttctccatgaggcgttgcatagtcactatacacagaagagtcttagtttgtctccaggagaaggaggttctgattcttggcaagaggaggtaatcaaattgtgtggccgagaacttgttagagctcagatagccatctgcggacagtctacggcgtccgatgcggccggagctaatgctgacgcaggtgcgcgacagctgtactccgcactggcgaataagtgctgccacgtgggatgcactaagcggtctctcgcgcaattctgt

SEQ ID NO: 426 - DNA, 885 bp, synthetic construct
  source 1..885: mol_type = other DNA
gataaaacccacacctgtccaccatgcccggcgccggaagccgccgggggacccagcgtatttcttttcccccccaagcccaaagacacgctgatgatttcacgaacgccggaggtgacttgcgtggtagtggacgtctcccatgaggatcccgaagttaaatttaattggtatgtagatggtgttgaggtccataatgctaaaacaaagccgcgggaagagcaatataactccacctatagagtggtctctgtactcactgtcctgcaccaggattggctgaatgggaaagagtacaagtgtaaagttagcaacaaagcgctcgccgcgcctatcgaaaaaacgatttccaaagcaaagggccaaccacgagaaccccaggtttacaccctgccacccagtcgagatgaactcactaagaatcaggtgtcccttacatgcctcgtcaagggattctatccgagcgatatagcggtggaatgggagagtaacggtcaacccgaaaataactataaaaccactccgccggtactcgattctgacggttccttctttctttattccaaactgactgtagacaaatcacggtggcagcagggcaacgtgtttagctgctctgtactccatgaggccttgcattctcattatactcaaaagagtctgagtctgagtccaggtgaagggggttccgattcatggcaagaggaagtcattaaactctgcggaagggaacttgtaagagcacaaatcgcgatttgtgggcaatctaccgcatccgacgcggctggagcagatgcagatgccggagcgaggcagctgtattcagcattggctaacaaatgttgccatgttggatgtacgaagagatcacttgcacagttctgt

SEQ ID NO: 427 - DNA, 885 bp, synthetic construct
  source 1..885: mol_type = other DNA
gataagactcatacctgcccgccctgtcccgcacccgaggctgccggagggccatcagtgttccttttcccaccaaagccgaaggatacacttatgatcagcaggacacccgaagtgacctgtgtagtcgtagacgtgtcccacgaagaccccgaagtaaaatttaattggtatgtcgatggcgtagaggtccacaacgcgaaaacgaaaccccgcgaagaacaatataattccacataccgagttgtcagcgtcctcactgttctccatcaggactggctgaatgggaaggaatataagtgcaaggtctcaaacaaggcgctggcggcccccatagagaaaacgatttctaaggccaaaggacagccacgggaaccgcaggtctatacgctcccacctagtagggatgagttgaccaagaatcaggtatccctcacatgtctcgtcaagggattctatcccagcgacatagccgtggagtgggaatctaacggtcaacctgagaataactataaaacaacccccccggtcctcgactccgatggtagcttctttctgtattccaaactgacggtagataaaagccgatggcaacagggtaacgtctttagttgttctgtattgcacgaggcgctccatagtcactacacacagaagtctttgagcctctcacctggggaggggggtagcgattcttggcaggaggaagtgatcaaactgtgcggcagggaactggtcagagcacagatagcaatatgcggtcagagtacggcctctgacgccgccggtgcggaggctgaggcaggggcgagacagctctacagcgctcttgcaaataagtgttgtcacgtggggtgcacaaagagatccttggcgcaattttgt

SEQ ID NO: 428 - DNA, 885 bp, synthetic construct
  source 1..885: mol_type = other DNA
gataagacccacacatgtccgccatgtccagccccagaggcagcagggggcccgtccgtattcttgtttcccccgaaacccaaagatacccttatgattagtcgaactccagaagtcacgtgtgtggtggtggacgtatcccacgaggaccccgaagtgaaattcaattggtatgtggacggggtggaagtccataacgctaagacgaagcccagagaggagcagtacaattctacctatcgggttgtatctgtgcttactgttctccatcaagattggctgaacgggaaggaatacaaatgtaaagttagtaacaaagcattggcagctcctatcgaaaagacgataagcaaggctaaaggtcaaccccgagagcctcaggtctacactttgccgccctccagggatgagcttaccaagaaccaagtgagcttgacgtgtctcgtgaagggattctacccatcagatatagcggtagaatgggagtctaatgggcagcccgagaacaactataagaccacccctcccgttcttgactccgacggttcctttttcttgtactccaaactcacggtcgacaagtctaggtggcagcaaggcaatgttttcagttgttccgtgctgcacgaagctcttcattctcactatacgcaaaaaagcctgagtctttcacctggagaggggggttccgattcttggcaggaagaagtcattaagctgtgcggcagagaacttgtgcgcgcacaaattgctatttgtggacagtcaactgcatctgacgccgctggagcccaagcggacgcaggggcaaggcagctttattcagcgcttgcgaataagtgttgccatgtgggttgcacgaaacgaagcctggcgcaattttgt

SEQ ID NO: 429 - DNA, 885 bp, synthetic construct
  source 1..885: mol_type = other DNA
gataagacacatacatgtcctccctgtcccgctccggaggcagccggtgggccttcagttttcttgtttccgccgaagcctaaggacacgttgatgatatcccgaacaccagaggtcacatgcgtcgtcgtggacgtctcacacgaggaccctgaagtgaaattcaactggtatgtagacggggtcgaagttcacaatgcgaaaactaaacctcgcgaggagcaatataactcaacataccgcgtagtgtccgtcttgactgtccttcatcaggattggctgaatggtaaagaatataaatgtaaagtttctaataaagcgcttgcggcacccattgagaagacaatttccaaagccaaaggccaaccccgagagcctcaggtatatacgctgcctccgtctcgagatgagttgacaaaaaatcaagtcagcttgacttgtcttgtaaaggggttctatccgtcagacatagcagtggagtgggaatccaacgggcaaccagaaaataattacaaaaccactccgcccgtgcttgactcagatgggagcttcttcctttatagcaaacttacggtagataaatccagatggcagcaaggcaacgtattcagctgtagtgtgctgcatgaagcgcttcactcccattatactcaaaaatctctttctctgtcaccgggcgagggcggaagtgatagttggcaggaagaggtcatcaagctctgtgggagagagcttgtacgcgctcagattgctatatgcggccagtcaactgcaagcgatgcagcgggtgccgatgcccaagcgggggcacggcaactctactcagccctcgcgaataaatgttgtcatgtagggtgtactaagagaagcctcgcgcaattttgt

SEQ ID NO: 430 - DNA, 885 bp, synthetic construct
  source 1..885: mol_type = other DNA
gataaaacgcatacttgcccgccgtgcccagcacctgaggcagccggcggccctagtgtcttcttgttcccgcccaagcccaaggatacactcatgatctcccgaacgccagaggtcacatgcgtagttgttgacgtttcccatgaggaccctgaagtgaaatttaactggtacgtcgacggcgttgaggttcacaacgctaagactaagccaagagaggaacagtacaattcaacttatagagtggtgtctgtattgacagttctccatcaggattggctgaacggaaaagaatataagtgcaaggtctcaaataaggcgctcgctgcacccatagaaaaaaccatatcaaaagcgaaggggcaaccaagagaaccccaggtgtacacgctccccccgtccagagatgaactcacgaagaatcaagtgtcactcacatgtcttgtaaaggggttctacccctctgatattgccgtagaatgggaaagcaacggacagcccgagaataactacaagacgacaccgccagttcttgattctgacggaagctttttcctctattcaaaattgaccgttgacaagtcccgatggcaacagggcaacgttttctcatgctccgtccttcacgaagccttgcattcccactatacgcagaagagtctctctttgagccccggagagggaggcagtgattcatggcaagaggaagtgatcaaactttgcggcagagaattggttagagcccagattgccatttgtggacaaagtacggcctcagatgctgcgggggcacaagctcaggcgggcgcacgccagttgtacagtgctctggcgaataagtgctgccacgttggttgcaccaagcgatccttggcgcaattttgc

SEQ ID NO: 431 - DNA, 885 bp, synthetic construct
  source 1..885: mol_type = other DNA
gacaagactcatacttgtccgccctgccccgctcctgaggctgccggaggcccttcagtattcttgtttccgccgaaaccgaaggataccttgatgattagtaggacaccggaagtcacctgcgtagtggtggacgtaagccacgaagatcccgaagtaaagtttaattggtatgttgatggcgtagaggtgcataatgcgaaaaccaaacctagggaggaacagtacaatagtacttaccgcgtagtgtcagtgcttaccgtgctgcatcaggactggcttaatgggaaggaatacaaatgtaaagtatccaataaagcgctggcggctcccatcgagaaaacgatctcaaaagccaaaggacaaccacgggaaccgcaggtctatactctgccaccttcaagagacgaacttaccaagaaccaagtctcattgacgtgcttggtaaaaggtttttatccgtctgacatcgctgttgaatgggagtctaacggccagccggagaacaattacaaaacaactccaccagtcttggattcagatgggtctttttttttgtattcaaagcttaccgttgacaaaagccgctggcaacaaggaaacgttttcagctgcagtgtgctgcacgaagcgctccacagtcattatacccagaaatctttgagcctgtctccaggggaaggtgggagtgactcttggcaagaagaggttatcaaactttgcgggcgggagctggtaagggcccaaattgcaatatgcggcaaaagtactgcatctgatgccgctggggccgatgctaacgcgggcgcaagacaactttatagcgcgttggcgaacaaatgctgtcatgtgggatgcaccaaacaaagtttggcgcaattttgt

SEQ ID NO: 432 - DNA, 885 bp, synthetic construct
  source 1..885: mol_type = other DNA
gataaaactcacacgtgtccgccatgccccgcacctgaagcggcgggtggtccgagcgtgtttttgtttccgcctaagcccaaggataccctgatgattagtcggacacccgaagtaacatgtgtcgtcgtggatgtaagtcacgaggatcccgaagtgaaattcaactggtatgtggatggagttgaagtccataatgcgaaaacaaaaccgagagaggaacagtacaactcaacataccgggtggtaagtgtactgacggtactccaccaggactggctgaatggtaaggagtacaaatgcaaagtttcaaataaggcgctcgctgcccccatcgagaaaaccattagtaaggctaaaggtcaacctagggagccacaagtatatacattgccgccttctagagatgagctgaccaaaaaccaggtcagcctgacctgtttggtgaaaggcttctatccaagcgacattgctgtcgagtgggagtcaaatgggcagccggaaaataactataaaacgactcctcctgttctcgactccgatggttcattcttcctctactcaaagcttaccgtggataaatccaggtggcaacaaggtaacgtgttctcatgttccgttctgcacgaagcactgcattcccattatacacaaaaatccctgagcctctcacctggggagggcggaagcgatagttggcaagaggaagtaataaagctgtgtggcagggaactcgtaagggctcagattgcgatatgtggaaaaagcactgcttctgacgccgcaggggccaacgcagatgctggcgcccgacaactctattctgcgcttgcgaacaagtgttgtcatgtaggatgtaccaagcaaagccttgctcagttctgt

SEQ ID NO: 433 - DNA, 885 bp, synthetic construct
  source 1..885: mol_type = other DNA
gacaaaacccacacttgtccgccctgtcccgctccggaggctgcaggcggcccaagtgtgtttcttttccccccaaagccgaaagacaccttgatgatatcccgcacacccgaagtgacttgcgttgtcgtcgacgtgtctcatgaggacccagaagtcaagtttaattggtacgttgatggcgtggaagttcacaatgcgaaaactaagcccagagaggagcaatataactcaacctaccgggtggtaagtgttctgacagttctccaccaggactggttgaacggaaaagaatacaaatgcaaagtgagtaacaaagccctggctgcccctatcgaaaagaccatatccaaagcgaagggccagccacgggaaccgcaagtatatacacttccaccatctagagatgagcttacaaagaaccaggtgtcccttacctgccttgtcaaaggcttctatccctctgacatcgcagtggagtgggagtccaacggacaaccagagaacaactataagacaacgccgccagtactggattcagatggttcattcttcttgtattctaaactgactgttgataaatcccgatggcagcagggcaacgtttttagttgtagtgttctgcacgaagcccttcattcccattatacacaaaaatctctttccctcagcccaggcgagggaggaagtgacagttggcaagaggaggtgataaagctctgtgggagggagctggtacgcgcacagattgcaatctgcggaaagagcacagcaagcgatgctgctggggccgatgccgatgctggcgctcgacaattgtattcagctcttgctaacaaatgctgtcacgtaggatgcactaaacagagccttgctcaattttgt

SEQ ID NO: 434 - DNA, 885 bp, synthetic construct
  source 1..885: mol_type = other DNA
gataagacccacacatgtccaccatgcccagccccagaagcggcaggtggtccttctgtgtttctctttcctcccaaaccgaaagatactctgatgataagccggaccccagaagttacgtgcgttgtagtagacgtgtctcacgaggacccagaagtgaagtttaactggtatgtcgacggtgtagaagttcataatgcgaaaacaaagcccagggaagaacaatataattcaacgtaccgggtcgtttccgtgctgacagttctgcaccaagattggctcaacgggaaagagtacaaatgcaaagtatcaaataaggccttggctgcgccgattgaaaagacgatttccaaagcaaagggccagccaagggaaccccaggtctataccctccctcctagcagagatgaacttacaaaaaaccaagtctccctcacctgcctggtcaaaggattctatccctcagatatagcagtagaatgggaaagtaacgggcagcccgaaaacaattataagaccactcctccagtactcgattcagacggtagcttctttctgtattccaagctgaccgtagataaaagtaggtggcagcaaggtaatgtcttctcatgtagtgtacttcatgaggcgttgcattcccattacacgcaaaagtctttgagtctcagtccgggtgaaggaggtagcgattcttggcaggaagaagtaattaagctgtgcggccgggagctcgtcagggctcagatagctatatgcggcaagagcacggccagtgatgctgctggtgcagaggctgaagcaggtgccaggcagttgtacagcgcactcgctaataagtgttgccacgtggggtgtacaaagcaatctttggcacaattctgt

SEQ ID NO: 435 - DNA, 885 bp, synthetic construct
  source 1..885: mol_type = other DNA
gataagacccatacttgtcctccgtgcccggcaccagaggctgcgggtggcccatccgttttcctgtttccgccaaagcctaaggatactctgatgatttcacgcacacccgaagtgacctgcgtggtggtcgacgtatctcacgaagacccagaggtaaaattcaattggtacgtggacggcgtcgaggttcataacgcgaaaactaagccgagagaagagcagtacaactctacgtatcgcgtggtgtccgtactgacagtattgcatcaggactggttgaatggcaaggagtataagtgcaaggtatctaataaggcattggctgccccaatagagaaaacgatcagcaaagcaaaggggcagccgcgcgagccgcaggtatatacacttccaccatcacgggatgagttgacgaaaaatcaagtctctctcacatgtctggtaaaaggtttctatccttctgatatcgccgtggaatgggaaagcaacggccaacccgaaaacaactataagacgacgccgccggtactcgacagcgacggaagctttttcttgtattccaagttgacagtggacaagtctcgatggcagcaaggaaacgtgttctcatgttctgttcttcacgaagcccttcatagccattatactcagaaatctctctcactctccccaggtgaagggggaagtgactcttggcaagaagaagtcattaagctttgcggtcgagaattggttcgggctcaaatagctatttgtggcaagtccacggcaagtgatgcagcgggggctcaggcagacgcgggcgcaaggcagctttattccgcacttgcaaataagtgctgtcacgtcggatgtactaaacaatcacttgcacaattctgc

SEQ ID NO: 436 - DNA, 885 bp, synthetic construct
  source 1..885: mol_type = other DNA
gacaagacacatacatgtcccccatgccctgcacccgaagctgctggggggcccagcgtgttcctgtttccgccgaagcccaaggacacattgatgattagtagaaccccagaggtaacttgtgttgtggtcgatgtgtcacatgaagaccccgaggtaaagtttaactggtatgtggatggggtagaggtacataatgcaaaaaccaagccgcgggaggagcagtataattcaacctatcgagtcgtgtcagtcttgaccgtgctccaccaggactggcttaacggtaaggagtataaatgcaaagtcagtaataaggcattggccgcccccattgagaagaccatcagtaaagctaaggggcaacctagagagccacaggtttacaccctccctccctcccgggatgaactcaccaaaaaccaggtgtcccttacttgtttggtaaagggcttttatccttctgatattgctgttgaatgggagtctaacgggcaacctgaaaataactacaaaacaactccccccgttctggactctgatgggtcattcttcctttattcaaaattgacagttgataagagtagatggcaacaaggcaacgtattttcatgttctgtgctccacgaggctctccattcccactacacacagaaaagtctctcactgtccccaggagagggcgggagcgactcttggcaggaagaagtaatcaagttgtgtggcagggaactcgtacgcgctcagattgcaatatgcgggaaatccacggcaagtgacgctgccggggccgacgcgcaagcaggggcacggcagctttactccgccctcgcaaataaatgttgtcatgtgggatgcactaaacagtcccttgcccagttttgc

SEQ ID NO: 437 - DNA, 885 bp, synthetic construct
  source 1..885: mol_type = other DNA
gataaaacccatacctgtccaccatgccccgcgccagaggcagcgggtggtccaagcgttttcctttttccaccgaaaccaaaagatacacttatgatatcaaggacccccgaggtaacgtgcgtcgtagttgacgtttctcacgaagatcccgaggtgaaattcaattggtacgtagatggtgtagaggtacacaatgcgaagacaaaaccgcgggaagagcagtataatagcacatacagagtcgtgagcgtcctcaccgtacttcaccaagattggctgaatggaaaggagtacaaatgtaaggtaagtaataaagcacttgcggcccccatcgagaaaactatcagtaaagcaaaagggcaaccacgagaaccccaggtctacactttgccaccatcacgggatgaactgacaaaaaatcaggtgtcactcacttgccttgttaaagggttctatcctagtgacatagcggtagaatgggagtctaacgggcagcctgagaacaattataaaactacgccccctgttcttgattccgatggatcattttttctctactccaaactcaccgtagacaaatcccgctggcagcagggcaacgtgtttagttgcagcgttcttcacgaagcacttcactcacattacacacaaaagtccctgagcttgagtcctggggagggtggatctgattcttggcaggaagaagttataaaactttgtggcagagagttggtccgcgcacaaatcgccatatgtggtaaaagcacagcgtctgacgcggcgggagcgcaagcccaggcgggggctcggcaactctactcagccctggctaacaagtgctgtcacgtgggatgcactaaacaaagtctggcgcagttctgc

SEQ ID NO: 438 - DNA, 885 bp, synthetic construct
  source 1..885: mol_type = other DNA
gacaaaacccatacgtgtcccccgtgtccggctccagaggctgcgggaggaccgtctgtgttcttgttcccgccgaagcctaaagatacgctgatgattagtcggacccccgaggtgacctgcgtggtagtagacgtatctcatgaagatccggaagtaaagtttaactggtacgtagacggcgtcgaggtacataatgccaagacgaaacccagagaagagcaatataatagcacttatcgagttgtaagcgtattgacggtccttcaccaggactggttgaacggcaaagagtacaaatgtaaggtatccaataaagcattggctgcgccaattgaaaagacaatttccaaagcgaaggggcaacctcgagagccgcaagtctacacgctgccaccgagtagggatgaattgactaagaatcaggtgagtctcacgtgtctcgtgaaggggttttaccccagtgatattgcggtagaatgggagtccaacggtcagccagaaaataattataaaacaacgccccctgtattggattctgacgggagctttttcctgtactcaaaactcaccgtagataagagtcgctggcaacagggcaacgtattctcatgtagcgttctgcacgaggcgctgcactctcactacacacagaagagtttgagtttgtcccctggcgaaggaggttctgattcctggcaggaggaggtgattaagctgtgtggccgcgaattggtgagggctcaaattgctatttgcggacagagcacagcgtccgatgccgccggcgcagatgctaatgccggtgcaaggcaactgtactccgctctcgccaataagtgttgtcatgtcggctgcaccaagcaatccctggcccagttttgc

SEQ ID NO: 439 - DNA, 885 bp, synthetic construct
  source 1..885: mol_type = other DNA
gacaagactcacacttgtcccccatgtccagcaccggaagctgccggcggtccctcagttttccttttcccccccaaacccaaggacacccttatgatttcaaggacaccagaggtaacgtgcgtagtggtggacgtcagtcatgaagacccagaggtaaagtttaactggtacgtggatggggtagaggttcataatgctaaaacaaaaccacgcgaggaacagtacaatagtacgtatagagtggtctccgttcttacggtgctgcatcaggactggctgaacggaaaagagtacaagtgtaaggttagcaataaggcgctggcggccccaatcgaaaagacgatttctaaggccaaaggccagccaagggagccacaagtatatacccttcccccttcccgagatgagctgactaagaatcaagtcagtctcacctgccttgtcaaagggttctacccatccgatattgctgttgaatgggagtctaatggccagccggagaacaattacaagacaactccgcctgtattggattccgacgggtcttttttcctctattcaaaactcacagtagacaaaagtcgatggcagcaaggtaacgtgttttcttgctctgtgttgcatgaagcacttcattctcattatactcaaaaatcattgagcctcagtccaggcgaagggggtagtgactcatggcaggaggaggtaatcaagctttgcggacgagagttggtcagggcccagatagctatttgtggtcagtccacggcgagtgacgcagcaggggcgaatgccgatgcaggagcaagacaactgtattctgctctggccaacaagtgttgtcatgtagggtgtactaaacaaagtctcgcccagttctgc

SEQ ID NO: 440 - DNA, 885 bp, synthetic construct
  source 1..885: mol_type = other DNA
gataaaacccacacctgtcccccatgtccggctcccgaagcagcggggggcccttcagtttttctctttccccccaaaccgaaagacacgctgatgattagcagaactccagaggttacctgtgtagttgtggacgtttcacacgaggatcccgaggttaaattcaactggtatgtggacggcgtcgaagtgcataatgcaaaaacaaagccccgggaagaacaatataatagtacctatagggtcgtttccgtactgaccgtacttcatcaagattggctcaatgggaaggaatacaaatgtaaagtgagtaataaagccctggcggcaccgatcgaaaaaaccatttcaaaggctaagggacaaccgcgcgaacctcaggtctataccttgcccccttcacgcgacgagcttacgaagaatcaggtaagccttacttgtcttgtcaagggtttttaccccagcgacatagctgtcgagtgggaatccaatggccaaccggagaataattacaaaactacccctcctgttcttgatagcgacggaagcttcttcttgtattccaaactcacagtagataaaagtaggtggcagcagggcaatgtattttcttgcagcgtcctgcatgaagcactgcatagccattatactcaaaagtccctgtctttgtctcctggagagggcggaagcgattcttggcaagaggaagttattaagctgtgcgggcgcgaacttgtgagggctcaaatagcgatatgtggtcagagcaccgctagcgatgcggctggtgcagacgccgatgccggtgctaggcaactttacagtgcacttgcgaataagtgctgtcacgtcggatgtactaaacaaagcctcgcccagttctgc

SEQ ID NO: 441 - DNA, 885 bp, synthetic construct
  source 1..885: mol_type = other DNA
gataaaacacatacttgccctccttgtccggctcccgaagccgcaggtggaccttccgtctttcttttcccacccaaacctaaagacactttgatgattagccggacccccgaggtaacctgtgtcgtagttgacgtttcccatgaagaccccgaagttaagttcaactggtatgtcgacggcgtcgaggtgcacaacgcgaagactaagccaagagaggagcaatacaattcaacttacagggtcgtgtccgtcttgacagtgcttcatcaagactggcttaatggaaaggaatacaaatgtaaagtctccaacaaggctctcgcagcgcccattgagaaaacgatatccaaagcgaagggtcaaccaagagaaccccaggtttacaccctcccccctagtcgggacgagcttacgaagaaccaggtcagtttgacatgcctggtgaaaggcttctatccgtcagacatcgccgtagagtgggaaagcaacgggcaacccgagaacaactataagacgactcccccggtgttggatagcgatggctctttcttcctgtactctaagctgaccgtagataaatccaggtggcaacaggggaacgtgttttcatgctcagtgctccatgaagccctccattcacactatacacaaaagtctttgtcactgtcccccggtgaaggcggcagtgatagctggcaagaagaagtcataaagctctgtggtcgcgagcttgttagggcccaaattgcgatctgtggtcagtcaacggcttctgacgccgccggagcggaagccgaggcgggtgctcggcaattgtattcagcactggcgaacaaatgttgccatgttggttgtactaaacaaagcctggcccagttttgc

SEQ ID NO: 442 - DNA, 885 bp, synthetic construct
  source 1..885: mol_type = other DNA
gacaagactcacacttgtccaccctgtccagcacctgaagccgctggtggaccatctgtctttctgttcccccctaaaccaaaggatacacttatgatcagcagaacacctgaagtcacatgcgttgtggtagacgtttcccacgaggatcctgaagtgaagtttaactggtacgtggatggcgttgaggttcataatgccaagacgaaacctcgggaggagcagtataattctacttatagggtggtaagcgtactgacagtcctccatcaagactggttgaacgggaaggaatacaagtgtaaagtttccaacaaagctctggcggcgcctatagaaaagacaatatcaaaagcgaaagggcaacccagagagcctcaagtatatacattgccccctagcagagacgaattgacgaaaaatcaggtctctctcacgtgcctcgtgaagggcttctatcctagtgatatagctgtggaatgggaatccaatggacagccagaaaacaactacaagaccacgccccccgtcttggattccgacgggtcattcttcctgtacagcaagctgactgtcgacaagagtcgatggcaacagggcaacgtctttagctgcagcgtcctgcacgaagctctgcatagtcattacacccaaaagtccctttctctctcccctggtgaaggcggttccgattcatggcaagaagaagtaattaagctctgtggacgagagcttgtccgagcacaaattgcgatctgcgggcagagtaccgcatctgacgctgctggcgcgcaggcagatgcgggtgcacggcagctttattcagctctcgccaacaagtgttgtcatgtggggtgtacaaagcagagccttgcccagttttgt

SEQ ID NO: 443 - DNA, 885 bp, synthetic construct
  source 1..885: mol_type = other DNA
gataagacccatacgtgtcccccttgccctgcacccgaggcggctgggggcccttccgtattcttgtttcctcctaagcccaaagataccttgatgataagtcgaacgccagaagtgacttgcgttgttgtggatgtctcccacgaggatccagaagtcaaatttaactggtatgtcgatggggtcgaagtgcataatgctaaaacgaaacccagagaggaacaatacaattcaacataccgcgtagtcagtgttcttactgtgctccatcaggattggctcaatgggaaagaatacaagtgtaaagtctcaaataaagcattggcggcccctatagagaagaccataagcaaggctaaaggtcagcctagggagcctcaagtatataccttgcctcctagcagagatgagttgaccaagaaccaggtcagcctcacatgcctggtgaaagggttttacccatctgatattgccgtcgagtgggaaagtaatgggcagccagagaacaactacaagacgacaccaccggtactggatagtgacggaagtttttttctttacagtaagctcacagtcgacaaaagccggtggcaacaaggaaatgtattttcatgtagcgtacttcatgaagccctccactctcattacacgcagaagtcactttcacttagtccgggtgagggtggaagcgatagctggcaagaggaggttatcaagctctgtggacgagaactcgtgagagcgcaaattgcaatctgcgggcagagcacggcgagtgatgcggccggggcggacgcgcaagcaggagcacgacaactttatagtgctttggctaataaatgttgccacgttggatgtactaaacagagcttggcacagttttgc

SEQ ID NO: 444 - DNA, 885 bp, synthetic construct
  source 1..885: mol_type = other DNA
gataaaacgcacacttgtccgccatgtccggcacctgaggcagcgggaggaccgtccgtgtttctgtttccccctaaaccaaaggacacgctgatgatcagccgaacacctgaagtaacatgcgtggtcgttgacgtgtctcacgaggatccagaagtaaagttcaattggtatgttgacggagttgaagtacataatgctaagactaaaccccgcgaagaacaatataattctacgtacagagttgtatccgtgctcacggtacttcaccaagattggcttaacgggaaagaatataagtgtaaggtctcaaataaggccctggctgctccgatcgaaaaaacgatatcaaaggcaaagggtcaacctcgggagcctcaagtatataccctccccccatctagggatgagctgacaaagaaccaagtttcactgacctgtctcgtaaagggtttctatccttctgacatcgcagttgaatgggagtccaacggccaaccagagaacaactataagacgacaccccccgtgttggacagtgacggaagttttttcctgtactccaagctgacggttgataaaagtagatggcaacaaggaaatgttttcagttgttctgtgttgcacgaggccctccactcacactatacccaaaaaagtttgtctctgagtcccggtgaaggcgggagcgattcatggcaggaggaagtaatcaaactttgtgggcgagaactggtcagggcgcaaatagcgatatgtgggcaaagcacagcttcagatgcagccggtgctcaagctcaggctggagctcgacagctttatagcgccttggctaataaatgttgtcacgttggctgtacgaagcagagcctggcacagttctgc

SEQ ID NO: 445 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcacgttgggtgtaccaagcggtcccttgcgcaattctgc

SEQ ID NO: 446 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatttcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcacgttgggtgtaccaagcggtcccttgcgcaattctgc

SEQ ID NO: 447 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcacttcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcacgttgggtgtaccaagcggtcccttgcgcaattctgc

SEQ ID NO: 448 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcaattcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcacgttgggtgtaccaagcggtcccttgcgcaattctgc

SEQ ID NO: 449 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatggcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgttacgttgggtgtaccaagcggtcccttgcgcaattctgc

SEQ ID NO: 450 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatggcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcttgttgggtgtaccaagcggtcccttgcgcaattctgc

SEQ ID NO: 451 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatggcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtcaagttgggtgtaccaagcggtcccttgcgcaattctgc

SEQ ID NO: 452 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcatggcaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtaaagttgggtgtaccaagcggtcccttgcgcaattctgc

SEQ ID NO: 453 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgttacgttgggtgtaccaagcggtcccttgcgcaattctgc

SEQ ID NO: 454 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtaaagttgggtgtaccaagcggtcccttgcgcaattctgc

SEQ ID NO: 455 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacgggaggagaaggctctggaggcgaaggagaagggggcggaagacagctttactctgctctggctaacaagtgttgttacgttgggtgtaccaagcggtcccttgcgcaattctgc

SEQ ID NO: 456 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacgggaggagaaggctctggaggcgaaggagaagggggcggaagacagctttactctgctctggctaacaagtgttgtaaagttgggtgtaccaagcggtcccttgcgcaattctgc

SEQ ID NO: 457 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacgggaggagaaggctctggaggcgaaggatctgggggcggaagacagctttactctgctctggctaacaagtgttgttacgttgggtgtaccaagcggtcccttgcgcaattctgc

SEQ ID NO: 458 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtgaaggcggaagcgactcataccaagaagaagtcattaaactttgtggacgggagctggttagggcacagattgctatttgtggtaagtctacgggaggagaaggctctggaggcgaaggatctgggggcggaagacagctttactctgctctggctaacaagtgttgtaaagttgggtgtaccaagcggtcccttgcgcaattctgc

SEQ ID NO: 459 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgttacgttgggtgtaccaagcaatcccttgcgcaattctgc

SEQ ID NO: 460 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacggctagtgacgcagcgggcgccgaagcggaagctggtgcaaggcagctttactctgctctggctaacaagtgttgtaaagttgggtgtaccaagcaatcccttgcgcaattctgc

SEQ ID NO: 461 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaagacagctttactctgctctggctaacaagtgttgttacgttgggtgtaccaagcaatcccttgcgcaattctgc

SEQ ID NO: 462 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaagacagctttactctgctctggctaacaagtgttgtaaagttgggtgtaccaagcaatcccttgcgcaattctgc

SEQ ID NO: 463 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaagacagctttactctgctctggctaacaagtgttgttacgttgggtgtaccaagcaatcccttgcgcaattctgc

SEQ ID NO: 464 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaagacagctttactctgctctggctaacaagtgttgtaaagttgggtgtaccaagcaatcccttgcgcaattctgc

SEQ ID NO: 465 - DNA, 885 bp, synthetic construct
  source 1..885: mol_type = other DNA
gataaaacacacacgtgtcccccctgcccggctccagaggcggctggtggtcccagcgtattcttgtttcctcccaaacctaaggatacgctcatgatatcccgcaccccagaagttacgtgtgtagtcgtcgacgtcagtcacgaagatccagaggtcaaatttaactggtatgtcgacggagtagaggtccacaatgcgaaaaccaagcccagagaagagcagtacaactccacgtatcgcgtcgtctccgtcctcaccgtactccatcaagattggctgaatgggaaagagtataaatgcaaagtatctaacaaggctctgccagctccgatagaaaagactatatcaaaggccaaggggcagccaagggagcctcaagtctatactttgcccccatctcgggatgagcttacgaaaaaccaggtcagccttacctgtcttgttaaaggtttttatccgagtgacatcgcagtggaatgggaatctaatggtcaacctgaaaacaattacaaaaccacaccgccagtattggacagcgatggtagtttttttctttactcaaaactgactgtagataaaagcagatggcagcagggcaatgtcttttcatgtagcgttatgcatgaggctcttcacaaccactatacccaaaagtcattgtctcttagtcccggaaagggcggaagtgattcttggaaggaggaggtaatcaagttgtgcgggcgagagttggtacgggcacagatcgcgatatgcggaaaatccacaggtgggggcgaaggaggaggtgagggtggaggtgaaggacgacagttgtattccgccttggcaaacaagtgttgccatgtgggttgcacaaaacgcagtcttgcccgcttctgt

SEQ ID NO: 466 - DNA, 888 bp, synthetic construct
  source 1..888: mol_type = other DNA
gataagacacatacatgccctccctgtccggctccagaggcagccgggggtccatcagtcttcctttttccgcctaaacctaaggatacactgatgatctctcgaacaccggaggtcacttgtgttgtcgttgacgtatcacatgaggatcccgaagtaaagttcaactggtatgtcgatggtgtggaggttcataatgctaaaactaaaccacgggaggagcaatataattccacatatagggtcgtgagcgtgttgacggtgcttcatcaagactggcttaatgggaaggaatataaatgcaaagtgtcaaataaagcacttcctgcgccaatcgagaaaacaattagtaaggcaaaggggcagccgcgagaacctcaggtgtacaccttgccgccttctagagacgagctcacaaagaaccaagtttccctgacttgcctcgttaaggggttttatccgtccgatatagccgtggagtgggagtcaaacggccaaccggaaaataattacaaaacgacacccccagtattggatagtgacggctcttttttcctttattctaagctgactgtggacaaaagccgctggcagcagggcaatgtcttttcatgcagcgtaatgcatgaagccctgcacaaccactacacgcaaaaatccctttccttgtcacccggcaagggcggctctgactcctggaaagaggaagttataaaactctgtggccgagaacttgttcgagctcaaatcgcgatttgtggtaagtcaacgggtgggggcgaaggtggaggcgagggtgggggagaaggaggaggccagttgtactcagctcttgcaaataagtgttgccacgttggttgtacgaagcggagccttgctcgcttctgc

SEQ ID NO: 467 - DNA, 885 bp, synthetic construct
  source 1..885: mol_type = other DNA
gacaaaacacatacttgtccgccttgcccggcacccgaagcggccggcggacccagtgtctttctcttcccacccaaaccgaaagacactctgatgatttccaggacgcctgaagtgacctgcgttgtagttgatgtatcacacgaggatcccgaggtcaagttcaattggtatgtagatggggtggaggtccataatgcaaagacgaagccacgggaggaacagtacaactctacgtacagagttgtcagtgttttgaccgtccttcatcaggattggctgaacggtaaagaatataaatgcaaggttagcaataaagctttgcccgcccctatagagaaaacgatcagtaaggcgaaggggcagcctagggaaccccaggtatataccttgccgccaagtcgagatgagctgacgaagaaccaagtgagtctgacatgcctcgtgaagggcttctatccgagcgatatcgctgtcgaatgggagagcaatgggcagcctgagaataactataaaacaacgccacccgtcctcgactccgatggctcattcttcctgtacagtaaacttacagtagataagagtagatggcagcagggtaacgtctttagttgctccgtgatgcacgaggcattgcacaatcattacactcaaaaatctctgtccctgagtccgggcaaaggcggttcagatagctggatggaggaggtcataaagctttgtggacgagaactcgttcgcgcccagatagctatttgtgggaaatcaaccgggggtggagaaggtggcggagaagggggaggcgaagggcgccaactgtattctgcattggctaataagtgctgtcacgtaggatgtacaaaaaggtctctggcgagattctgc

SEQ ID NO: 468 - DNA, 885 bp, synthetic construct
  source 1..885: mol_type = other DNA
gacaagacgcacacttgtccaccttgccctgcgccggaagctgctggaggccccagtgtctttttgttcccgcccaaaccgaaggacactttgatgataagtcgcacgcccgaggttacctgtgtggttgtcgatgtctcacacgaagatccggaggtgaagtttaattggtatgtagatggcgtggaggttcataacgccaaaacgaaacccagagaagaacaatataacagtacatatcgagtagtatccgttctcactgtcctgcatcaagactggttgaacgggaaggaatataagtgcaaggtgagcaataaagcactcccggccccaatcgaaaagaccatcagcaaagcgaaggggcaacctcgagaaccccaggtatatacgctcccccctagtcgggatgaacttactaaaaatcaggttagcctcacttgccttgttaaagggttctatcccagtgatattgccgtcgaatgggaatcaaacgggcagccggaaaataactacaagacaacccctcctgtgctcgatagcgatggctcttttttcctctacagcaaacttaccgttgataagagccggtggcaacaaggtaatgttttctcctgctccgttatgcatgaagcactccataaccattatacccaaaaaagcctgtcacttagtccgggtaaaggaggtagtgattcttggcaggaggaggtaatcaaactttgtgggagggagctggtacgagctcagattgctatatgtggaaaaagcacgggcggaggagaaggaggtggcgaaggcgggggtgaaggtcggcaactctactccgctctcgctaataagtgctgccacgtcgggtgtacgaagcgctccctggcgcgattctgc

SEQ ID NO: 469 - DNA, 885 bp, synthetic construct
  source 1..885: mol_type = other DNA
gataaaacgcacacgtgtccgccctgcccagcgcctgaagccgcaggcgggccgtccgtcttcctctttcctccaaaacccaaagacacacttatgatcagtaggaccccagaggtaacctgcgtcgtggtcgacgtttcccatgaagacccagaggtcaagttcaactggtacgtcgacggtgtcgaagtacataatgctaaaacgaagcctcgggaagagcagtacaactctacctaccgcgtcgtttccgtactcaccgtacttcaccaggactggcttaacggtaaagagtataaatgcaaagtatctaataaggctctcgccgcgccgattgagaagacaatttcaaaggccaaggggcagccgcgggagccccaagtgtataccttgcccccgtcccgagatgagctgactaaaaaccaagtaagcttgacttgcttggtcaaaggcttctacccttccgatatagctgtcgaatgggagtcaaatggccaaccagagaacaattataaaactacacccccggtcttggattctgatggctcattttttctctattctaaactgaccgtggataagtctcgctggcagcaaggtaacgtgttcagttgctctgttcttcacgaagcactgcacagtcattacactcagaagagtcttagcctgagccctggtaaagggggttctgattcctggcaggaggaagtaataaaactctgtggccgggagttggtacgggcgcagattgcgatatgcggtaagagcaccggcggaggcgaaggcggtggggaaggaggaggagaagggagacaactctattccgcattggcaaataagtgctgccacgtcgggtgtaccaaacgatcccttgcacggttctgt

SEQ ID NO: 470 - DNA, 888 bp, synthetic construct
  source 1..888: mol_type = other DNA
gataagacccatacgtgccccccttgccctgcgcctgaggcagcgggtggcccatcagtctttttgttcccgcccaagccaaaggacaccctcatgattagtagaacaccggaggttacgtgcgtcgtagtggatgtcagccacgaggatcccgaggttaagtttaactggtacgttgatggggttgaggtccataatgcgaagactaagccgagagaggaacagtacaattccacgtatagagttgtctctgtactgactgtgctgcatcaagattggcttaacggtaaggagtacaagtgcaaagtctctaataaggctcttcctgcacccattgagaaaactataagcaaagcaaaaggtcaacctcgcgaacctcaggtgtacacactgccaccctctagggacgagcttaccaaaaatcaagtatctcttacctgccttgtgaaagggttttatccctcagatattgcggttgagtgggagtctaacggacaacctgagaacaactataagactactcccccggtgcttgattcagacgggagtttttttttgtatagcaaacttaccgtcgacaaaagccggtggcaacagggcaatgtattcagttgttctgtaatgcatgaagctttgcataatcattacacccaaaagagtctttccctgtctcctggaaaaggggggtcagactcctggatggaggaggtgatcaaactgtgtgggagagagctcgtccgggctcagatagctatatgcggcaagtctacgggtgggggagagggcggaggagagggcggtggagaaggaggcggccaactctacagcgctctggccaataaatgttgtcatgtcgggtgtactaagcgctcactggcacgcttttgc

SEQ ID NO: 471 - DNA, 888 bp, synthetic construct
  source 1..888: mol_type = other DNA
gacaagacgcatacatgcccgccatgcccggcccccgaagctgctgggggaccatccgtattcctcttccctcccaaaccaaaagacacgttgatgataagtagaacaccagaggtaacgtgcgtggttgtcgatgtttcccacgaagatccggaggtaaaattcaattggtatgtagatggggtggaagtgcacaatgccaaaacaaagccgcgagaagaacaatacaatagtacttaccgggttgtgagcgtgctcacggtgttgcaccaagactggctcaacggcaaggaatacaagtgcaaagtatctaataaagctctgcctgcgccgatagagaagaccatcagtaaggccaaagggcagccccgagagccgcaagtttacactcttcctccgagcagagatgaattgaccaagaaccaagtaagtttgacgtgcctggtgaagggcttctacccctcagacattgcggtggagtgggaaagtaatggtcaaccggaaaacaactacaagaccacgccgcccgtcctcgactccgatgggtctttctttctttattcaaagttgacagtagataagtcaaggtggcagcaaggtaacgtgtttagttgtagtgtaatgcacgaggccctgcataatcattatacccaaaagagtttgagcctctcaccaggaaaaggcggatcagacagctggcaggaggaggtaattaaattgtgtggacgggagttggtcagggcgcaaatagccatctgcggtaagagcacgggtggaggagagggtggaggggaaggtgggggagaaggcggcgggcagctctattctgcactcgccaacaagtgttgtcacgtcggatgcacaaagagatctcttgctcgattctgc

SEQ ID NO: 472 - DNA, 888 bp, synthetic construct
  source 1..888: mol_type = other DNA
gacaaaacacacacctgtccgccttgcccggctcctgaagccgcgggtggccctagtgtgtttttgtttccgccgaaacctaaggataccctcatgataagccggacgcccgaggttacctgtgtcgtggtcgatgttagtcatgaggatccagaagtcaagtttaattggtacgtcgacggcgttgaagtccacaatgcaaaaactaaaccgcgagaagaacagtacaactccacctacagagttgtctcagttttgacagttctccatcaggattggctcaatggaaaggaatataagtgcaaggtcagcaataaagcgcttgccgcccctatagagaagaccattagcaaggcgaaaggacagccccgcgagccccaggtctatacgctgcctcctagcagagatgagctcacgaaaaatcaggtcagcttgacatgcttggtgaagggcttctaccccagtgacatcgcagttgaatgggagagcaacggccaacctgagaacaactacaaaacaacgcccccggttcttgacagcgatgggtccttctttctttactctaagcttacagttgataaaagcaggtggcagcaggggaatgtgttctcatgttccgtactgcatgaggctctgcattctcactacacccaaaaaagccttagcctgagccccggtaagggaggtagtgactcatggcaagaggaagtgattaagctctgcggccgggagttggtgagagcccaaatcgccatttgcggtaaaagtaccggagggggcgagggaggaggcgaaggtggaggtgaaggaggtggacagttgtactcagctcttgcaaataaatgttgtcatgttggttgcacgaaaagatctcttgcgaggttctgt

SEQ ID NO: 473 - DNA, 885 bp, synthetic construct
  source 1..885: mol_type = other DNA
gataagacgcatacttgtccaccgtgccccgcaccggaagcggctggtggtccatcagtttttctgttcccaccgaaacctaaggacacgttgatgatatcacggacaccagaggttacgtgcgtagtggtggatgtgagccacgaggatccagaagttaaatttaattggtacgtagatggagtggaggttcataatgcgaagacaaagcctcgcgaggaacagtataattccacctatcgcgtcgtatctgtgcttacggtacttcaccaagactggttgaacggtaaggaatataaatgcaaggtttccaataaagcacttcctgcgccaattgagaagacaatatccaaagctaaaggtcaacccagggaaccgcaagtctacactctccccccgtctcgcgatgaattgacgaagaaccaggttagtctcacctgcctggtcaaggggttttacccctctgacatagctgtagaatgggagtctaatggacagccagagaacaattacaaaacgacccccccggtcctcgattctgatgggagtttttttctttattcaaaattgactgtcgataagtcaagatggcaacagggtaacgtattttcttgcagtgttatgcatgaagcattgcacaaccactatacacaaaaatcattgagtttgagtcccggtaaagggggaagcgactcatggatggaagaagtaatcaagctgtgcgggcgagagcttgtgcgagctcagatagcaatctgtggtaagtctacaggtggagagggtggcggtgaagaaggcgggggagagggaggccagctttattctgccctggctaacaagtgctgtcacgttggatgcacgaagcgctccctggcccgattctgc

SEQ ID NO: 474 - DNA, 885 bp, synthetic construct
  source 1..885: mol_type = other DNA
gataagacgcatacttgtcccccatgtcccgctccggaagccgctggcggcccctccgtttttctgttcccgccgaaaccgaaagacaccctgatgatatcacgcactcccgaggtcacttgcgtggtagtcgatgttagtcatgaagatcctgaggtcaaattcaattggtatgtagatggcgttgaggtacacaacgcgaagacaaaaccccgagaagaacagtataactcaacctaccgcgtagtttcagttcttaccgtactgcaccaagactggttgaacggtaaagagtacaaatgtaaagtcagcaataaagctttgccagcacctatcgaaaaaaccatcagtaaggccaagggtcaacccagggagccgcaagtgtacactcttccccctagcagggatgaattgaccaagaatcaggtctctttgacgtgcctcgttaagggtttctatcccagcgatatagccgtagaatgggagtctaacggtcagccagaaaataactataagacaaccccgcctgttttggattccgacggctctttttttctctactctaagttgaccgttgataagagcagatggcagcagggaaacgtattttcttgttccgtgatgcacgaagccctgcacaatcactatacgcaaaagtctctgagcttgagtccgggtaaaggcggttctgactcctggcaggaggaagtcataaaactctgcggaagagagctcgtaagggcgcaaatcgctatttgtggtaagagcaccggtggggaaggaggcggtgaagagggtggcggcgagggtgggcaattgtattccgcgcttgccaataaatgttgtcacgtaggctgcacaaagcgaagtctcgctaggttctgc

SEQ ID NO: 475 - DNA, 882 bp, synthetic construct
  source 1..882: mol_type = other DNA
gacaagacccacacatgtcccccgtgtccggcaccagaagcagcggggggaccgtcagtattcttgtttccaccgaagcccaaagacacattgatgatttcacgaactcctgaagttacctgtgtggttgtagatgtatcacacgaagacccagaagtcaaattcaattggtatgtcgacggggttgaagttcacaatgcgaagacgaagccccgggaggaacagtacaacagcacgtacagggttgtgagcgttcttactgtattgcaccaggattggctcaacggcaaggagtataaatgtaaagtttctaataaggctcttcctgccccaattgaaaagacgatatctaaagcgaagggccaaccacgggaacctcaggtgtacacacttccgcctagcagggatgagttgaccaagaatcaagtctctttgacgtgcctggtcaaggggttttacccatcagatatcgccgtcgaatgggagtcaaacggacaacccgaaaataactataaaactactccaccagttctggatagcgacggctcattttttctgtattcaaagctcactgtagacaagtctaggtggcagcagggtaatgtcttctcctgctcagtaatgcatgaggctcttcacaaccactatactcaaaagagcctttccctgtcacctggcggtggaagcgactcatggatggaggaggtaataaagctctgcggaagagaactggtacgcgcacaaatcgcaatttgtggtaagagtactggcggggaaggaggtggggaagaagggggcggtgagggcggacagctctattctgcacttgcaaacaaatgttgccacgtgggatgtactaagcgaagccttgcaagattctgc

SEQ ID NO: 476 - DNA, 882 bp, synthetic construct
  source 1..882: mol_type = other DNA
gataaaacccacacatgccctccatgccctgctccagaggccgccggtgggccatcagttttcttgtttccgcctaaaccaaaggacacgcttatgatctccaggacccccgaagttacgtgtgtggtggttgatgttagtcacgaggacccggaagtcaagttcaactggtacgttgatggtgtagaggtgcacaatgcaaagacgaagccacgcgaagaacaatacaacagcacatatcgagttgtgagcgtactcacggtactgcatcaggactggctgaacggtaaagaatacaaatgtaaagtctccaataaggcacttcctgcgccgatagaaaaaacgatcagtaaggccaagggccaaccccgagaaccacaggtatatacgctcccaccgtcacgagacgagttgacaaaaaatcaggtctccctgacttgcctcgtgaaaggtttttatccctcagatattgctgttgagtgggaaagcaatgggcagccagagaataattataagacgactcctccggttttggattccgacggtagttttttcttgtatagtaagcttactgtagacaagtcaagatggcaacaaggtaatgtgttctcttgctcagttatgcatgaagctcttcataaccattacacgcaaaagagtctcagtctgagccccggtggcggtagcgacagttggcaggaagaggtgattaagttgtgcggtcgcgagctcgttcgggcccaaattgcaatctgcggaaaatctacgggcggagagggcgggggtgaggagggtgggggtgaaggtgggcagctctatagcgcccttgcgaataaatgttgtcacgtcggatgcacaaagaggtccctcgccaggttctgc

SEQ ID NO: 477 - DNA, 885 bp, synthetic construct
  source 1..885: mol_type = other DNA
gataagacccacacttgccccccttgccctgcccccgaagcggccggaggtccttcagtatttttgtttccaccgaaacccaaagatactttgatgatatcaagaactcctgaagtcacctgcgtggtagttgacgtatctcatgaggatcccgaggtgaagttcaattggtacgtcgatggcgtcgaggttcataacgctaagactaagccgagggaagagcaatataattccacttatagggtggtgtccgtcttgactgttttgcaccaggattggttgaacgggaaagagtacaaatgtaaggtgagtaataaagctttggctgctcccatcgaaaagacaataagcaaggccaaggggcaacctcgggagccgcaggtgtacacccttcctcccagtagagacgaactgacaaaaaaccaggtgtccctgacctgccttgtgaaggggttttacccgagcgacatagcggttgaatgggagagcaacgggcaacccgagaacaactacaaaactacaccgcctgtcctggactccgatggaagcttcttcctctactccaaactgaccgtggacaaaagcagatggcaacaaggaaacgtattctcatgctcagtaatgcacgaagcattgcacaatcactacacccaaaagtccctctcactctcccctggtaagggcggatcagactcatggcaagaggaggtaattaagttgtgcgggagggagctcgtccgcgcgcaaatagccatttgtggcaagtccactggaggaggcgagggtggaggagagggtggtggggagggcaggcaactctacagtgcgctcgccaataaatgctgccatgttgggtgcacgaagcgcagtctcgcacaattctgc

SEQ ID NO: 478 - DNA, 885 bp, synthetic construct
  source 1..885: mol_type = other DNA
gataagacccacacgtgtcctccatgtccggcaccggaggctgctggcgggccttctgtattcctcttcccacccaagccaaaagacacattgatgatatcaaggacgccggaagtcacctgtgttgttgtggacgtttcccatgaagacccagaggtaaaattcaattggtatgtggacggcgtagaggttcacaacgccaaaaccaaaccccgagaggaacagtataatagcacatatcgagtagtatctgttctcacagtgctccatcaagactggcttaatggtaaagagtataaatgcaaagtttccaataaagccctcgctgcaccgatcgagaagacaatcagtaaagcgaagggccagcctcgggaaccgcaggtgtatactcttccaccctcaagagacgagctcactaaaaaccaagtttcattgacatgcctcgtcaaaggtttctacccatcagacatcgcggtcgaatgggaaagtaatgggcagccggaaaacaactataaaacgacgccgcccgtcttggattctgatggttcattttttctttactctaaattgaccgtcgataaaagtaggtggcaacaaggaaatgttttttcctgctccgtcctgcatgaagcgttgcacagtcactatacccagaagagtctttctttgtcacccggaaaaggcggttcagattcatggcaggaagaagtaattaaactctgtggccgcgagcttgttagggcgcagatagccatatgtggtaaaagcaccggaggaggtgaaggcggaggcgaaggaggtggggaaggaagacaattgtattctgcacttgcaaataaatgctgtcatgtggggtgcacgaaacgcagtcttgcacaattttgt

SEQ ID NO: 479 - DNA, 882 bp, synthetic construct
  source 1..882: mol_type = other DNA
gacaaaacccatacctgccccccttgccctgcaccagaagcggcgggaggacctagcgtttttctttttcctccgaaaccgaaagataccctcatgatatcaagaacacctgaggttacttgcgttgtcgtggacgtgagtcacgaagaccccgaggtgaagttcaactggtatgtagatggagtggaggtccataatgcaaaaacgaaaccgagagaagaacaatacaactctacatatcgagtcgtgtcagtactcacggttttgcatcaagattggctgaacggtaaggagtacaagtgtaaggttagcaacaaggctctcgcggcgccgatagaaaagactataagtaaagcaaaaggccagcccagagaacctcaagtttacactctgcctcccagcagagatgaactgactaaaaatcaggtttcattgacctgtctcgtcaagggtttttatccaagcgacatagcagttgaatgggaaagcaacggtcaaccagaaaataattacaaaaccactccaccagtcttggactctgacggatccttctttctctattcaaaattgacggtggataaatctaggtggcagcaaggcaacgtcttctcttgtagcgttatgcatgaggcgctgcacaaccactacacacaaaagtctcttagtttgagcccgggcggcggaagcgactcttggcaagaggaagtgataaaactctgtggtcgagaattggtacgcgcgcagatcgctatctgcggcaagtccacagggggaggggaaggtggcggggaaggtggtggcgagggcaggcagttgtatagtgcacttgccaacaagtgctgccatgtggggtgcaccaagcgcagtttggcacggttctgc

SEQ ID NO: 480 - DNA, 882 bp, synthetic construct
  source 1..882: mol_type = other DNA
gataaaactcacacttgtcccccgtgtccggcaccagaagccgcaggagggccatctgtctttctttttcccccaaaacccaaggatacactgatgatctcccgcactcccgaagttacttgtgtcgtagtagacgtttctcacgaggacccagaggtgaaattcaattggtatgttgacggagtagaggtgcataatgccaagacaaagccccgagaggaacaatacaattcaacctacagagtagtgtccgttcttacggttctccatcaggattggctcaacggtaaggaatataagtgcaaggtaagcaacaaagcgctggccgcacccattgagaaaaccatttcaaaagctaaaggccaaccccgcgaaccacaagtttatactctccccccaagtcgcgatgaacttacaaaaaatcaagtctcattgacgtgcttggtcaaaggcttctacccgagcgatatcgctgttgaatgggagtctaatggacaaccggaaaataactataaaactacacccccagtcctcgattcagacggcagcttcttcctgtattcaaaactgacggttgacaaatcacgctggcaacagggtaacgttttttcctgtagcgttcttcatgaagccttgcacagtcactacacccagaagtcccttagcttgtcacctggcgggggttcagactcttggcaggaggaggtaatcaaactgtgcggaagagaactggtgagggctcagattgcaatttgtgggaagagcacgggtggcggtgaaggaggtggcgagggcggaggagaggggaggcaactctacagtgcgttggctaataaatgctgtcacgtcggctgtactaagagaagcctcgccagattttgc

SEQ ID NO: 481 - DNA, 882 bp, synthetic construct
  source 1..882: mol_type = other DNA
gacaagacgcatacttgccctccgtgccctgcaccagaagccgctggtggcccatctgtgtttttgttcccccctaagccaaaagacacattgatgatttcacgaactccagaagtgacttgcgtagttgttgacgtatcacacgaagaccccgaggttaaatttaattggtatgtggacggggtcgaggtgcataacgccaaaaccaaaccccgggaggaacaatataactctacgtatcgggtcgtatctgtgttgaccgtccttcaccaagattggttgaacggcaaggaatataagtgtaaagtgtctaataaagcattggctgccccgatagaaaagacgatctctaaagccaagggccaacccagagagcctcaagtatatactctcccaccgagtcgagatgagctcactaagaaccaggtgtcactcacgtgtctggttaaaggattttaccctagtgatatagccgtcgagtgggaatcaaatgggcagccggagaataactataagaccacgcctccagttctcgattccgatggtagctttttcctttactctaaacttacggtcgacaagtccaggtggcaacagggcaatgtattttcttgctccgtcatgcacgaggctttgcacaaccattacacgcaaaagtcactgtccctgtctcctggaggcggttctgacagttggcaggaggaggtaatcaaattgtgtgggcgggagttggttagggcgcaaattgctatttgcggcaaaagtactgggggcggtgaaggcggaggcgagggaggaggagaaggtcgacaactgtattctgccttggcgaacaaatgctgtcacgtcggctgtacgaaacggtctttggcccagttttgt

SEQ ID NO: 482 - DNA, 882 bp, synthetic construct
  source 1..882: mol_type = other DNA
gataagacacacacttgtccgccatgccctgcgccggaagcggcgggaggaccgtccgttttcctgttccctcccaaacccaaagacacgttgatgattagtcgcacgccagaagttacgtgcgttgtcgtagatgtatcccacgaagaccccgaggtgaagttcaattggtatgtagatggggtggaggtccataacgctaagaccaaaccacgcgaggaacaatataattctacgtaccgcgtagtgagcgttctcacagttcttcaccaggattggcttaacggcaaggagtataagtgtaaggtgtctaataaggccttggctgccccgatcgaaaaaacgataagtaaagcaaagggtcaacctagagaaccccaagtgtacactctcccgccatcacgggatgaattgactaagaaccaagtgtcactcacgtgtcttgtaaagggcttctacccatccgatatagccgttgagtgggaatccaatggtcagccagagaacaactataagacaactccgcccgtacttgatagtgacggttcctttttcctttacagtaaattgacggtagataagtctcgctggcagcaaggaaacgtcttttcttgttcagtgcttcatgaggcgcttcactcacactatactcagaagagtttgagtttgtctccaggtggaggcagcgactcatggcaagaggaagtaatcaaactgtgtggtcgcgaattggtacgagcacagatcgcgatctgcgggaaatcaacaggtggcggcgaaggcggcggggaaggcggcggcgaaggtaggcaactttactcagcccttgcgaacaaatgttgccacgtaggctgtactaagagaagtctcgcccagttttgc

SEQ ID NO: 483 - DNA, 885 bp, synthetic construct
  source 1..885: mol_type = other DNA
gacaagactcatacctgccccccttgtccagcaccagaagcagctggcgggccaagcgtgttcctgtttccacctaagcccaaagatacgttgatgatcagccgcaccccggaagtaacctgtgtagtagtagatgtgtcccacgaagaccccgaagtaaagtttaattggtacgtcgatggtgtcgaagtacataacgctaaaacgaagccccgagaagagcagtacaacagtacttacagagtagtttctgttcttacagtgctgcatcaggattggctgaacgggaaggagtataaatgtaaagtctcaaacaaggcacttgcggcaccaatagagaagacaatatctaaggccaaagggcagcctagagagccacaagtatatacgctgccccccagcagggacgagctgacaaagaaccaagtgtcactgacctgccttgttaagggcttctatccgagtgatattgctgttgaatgggaaagtaacggacagccggagaacaactataaaactactccacccgtgttggatagtgacggtagcttttttctgtactccaagttgacggtagacaaaagtcggtggcagcaggggaacgtattttcttgttctgtcatgcacgaagctcttcacaatcactatacgcagaagtccctctctctctctcctgggaagggtggttccgacagctggcaggaggaggtcattaaactgtgtggtagagagctggtacgggctcaaattgcaatttgtggtaagagtactggcggtggcgaggaagggggtggggaggagggcggaggtaggcagctctactctgctctcgccaacaagtgttgtcacgtcgggtgtactaaaagatcacttgcccgcttttgt

SEQ ID NO: 484 - DNA, 885 bp, synthetic construct
  source 1..885: mol_type = other DNA
gacaaaacacatacatgcccgccgtgtccggcgcctgaagcagcaggaggccccagtgtattccttttccctccaaagccaaaagatacgttgatgatatctaggacacctgaggttacctgcgtcgtagtggacgtatcccacgaagacccagaagtcaagtttaactggtatgtggacggagtggaggtacacaatgcaaagacaaagccgcgagaggaacaatataattccacctatagagtcgtgtcagtccttacggtcttgcaccaggactggctcaatggtaaggagtataagtgcaaagtatcaaacaaagctctcgcagcgcccatcgaaaagaccatcagcaaagctaagggccagccaagagagcctcaagtgtacacgttgccgccttcaagggacgagctcactaaaaatcaggtatcacttacgtgtcttgtcaaagggttttatccttccgacatcgcggttgaatgggagagcaatggacagccggagaataattataaaacgacgccgccggtccttgacagcgatggttcatttttcctttactcaaagctgacggttgataagtctaggtggcagcaggggaacgtcttttcctgtagtgtacttcatgaggcgctccattctcattacactcagaagtcactgagcctttcacccggcaaaggtggatcagactcctggcaagaagaggtaatcaaactctgtgggagggaactcgttcgagcccagattgcaatctgtgggaaaagcacaggcggaggggaagaagggggtggcgaagaaggtgggggcaggcagctctattcagctcttgccaacaaatgctgtcatgtaggctgcacaaagcgatcactggcgagattctgt

SEQ ID NO: 485 - DNA, 885 bp, synthetic construct
  source 1..885: mol_type = other DNA
gataaaactcatacttgcccaccctgcccggctcccgaggcagcaggtggaccctcagtatttttgttccctccgaaacctaaagatacacttatgattagccggacccctgaggtaacgtgtgtggtggttgacgtaagtcatgaagatccagaagtaaagtttaactggtacgtagacggtgtggaggtacataatgcgaagacaaaaccacgagaggaacagtataactctacctaccgcgtagtaagcgtacttactgtgctccaccaagactggcttaacgggaaagagtataagtgtaaagtcagtaataaagcactggccgccccgatcgaaaaaacaatcagcaaggccaaaggacaaccaagggagcctcaggtctatactcttcccccgagtagggatgagcttaccaagaaccaggtgtctctgacatgccttgtcaagggattttacccgagtgacatagccgtagaatgggagtcaaacggccaacctgaaaacaactataagaccacgcctcccgtactcgactcagatggaagctttttcctctatagcaagctgaccgtcgacaaaagtaggtggcaacagggaaacgtctttagttgttccgtcatgcacgaagctttgcataaccattacacccagaagagtctttccctttcccctggcaaggggggctccgactcctggcaagaggaagtaatcaaactgtgtgggcgcgagcttgtccgcgcgcaaatagccatttgcggaaaaagtactggaggaggagaggaaggcggcggcgaggaaggtgggggcaggcagctgtacagtgccttggctaacaagtgctgccatgtcggctgtacgaaaaggtctcttgctcaattctgt

SEQ ID NO: 486 - DNA, 885 bp, synthetic construct
  source 1..885: mol_type = other DNA
gataagacacatacctgtccaccctgcccagcacctgaagctgcaggcggccccagcgtattcctgtttcctccgaagccgaaagacacacttatgatttcccggacgcctgaggtaacttgcgtcgtagtagatgtgtctcacgaagaccccgaggtgaaattcaactggtacgttgatggtgtggaagttcataatgcgaaaactaaaccacgagaggagcaatataactcaacttatagagttgtgagcgtcttgacggtactgcaccaggactggctgaatggcaaagagtacaaatgcaaagtctcaaataaggcgttggcggctcccatagagaaaactatcagcaaagccaagggtcaacctcgggagccacaagtgtatactcttccgcctagtcgcgacgagctcacaaagaatcaggtgagtcttacttgtttggttaagggtttctaccccagtgacattgcggtcgagtgggaaagtaacggacagcctgaaaacaactataaaacaacgcctccagtactcgattcagatggttcattctttctttattccaaactcacagtcgacaagagtagatggcaacaagggaacgtgtttagctgtagcgtactccatgaggcactccactctcactatacccaaaagtctctcagcttgtcacccggaaaaggcggttctgacagttggcaagaggaagtgattaaattgtgtgggcgggaacttgtgagggctcaaatcgcgatttgcggcaagtccactggtggcggcgaggaaggaggaggtgaagaaggaggaggtaggcaactgtattcagcgttggcgaataaatgctgccatgttggatgtactaaacggagccttgctcagttctgc

SEQ ID NO: 487 - DNA, 882 bp, synthetic construct
  source 1..882: mol_type = other DNA
gataaaacgcatacttgccctccttgcccggcacctgaagctgccggaggtccttccgtgttcctgttcccacctaagccaaaagacacacttatgatttctcgcacaccagaagtaacgtgcgtcgtagttgacgtctcccatgaagacccggaggtaaaatttaattggtacgtcgacggggtagaagttcataacgcaaagactaaaccacgagaagagcaatacaactctacatacagagtagtaagcgttctcaccgttcttcatcaagattggctcaacggaaaggagtataagtgtaaggtgtccaataaagcgttggccgcaccaatcgaaaagaccataagcaaagccaaaggccaaccccgcgaaccgcaggtgtacacacttcccccgtccagggatgaattgacaaaaaaccaagtttccctcacgtgtctcgtcaagggattctacccgagtgatatcgcagttgaatgggaaagcaatggtcagcccgagaataactacaagactactccccctgtgttggactcagacggctcattcttcctctacagtaagttgactgtggacaaaagtcggtggcagcaaggcaatgtcttcagttgtagtgtaatgcatgaagcactccacaatcattacacccaaaaatccctgagcctgtccccgggcggaggttcagattcatggcaggaggaagttataaaactgtgcgggcgcgagttggtgagggcgcagatcgcaatctgtggaaagagtacgggaggtggcgaagagggtggtggagaagagggaggaggtcgacaactgtattccgcgctcgcgaacaagtgttgccacgttggctgcaccaaacgaagcctggctcgattttgc

SEQ ID NO: 488 - DNA, 882 bp, synthetic construct
  source 1..882: mol_type = other DNA
gacaagacacacacttgtccaccttgcccggctcccgaggcggcaggaggaccaagcgtttttctgttccctcccaaaccaaaggatacgcttatgatctctcgaacgccggaagttacttgcgtagtagttgatgtctcccatgaagatcccgaagtgaagttcaactggtatgtagatggtgtggaagttcataacgcgaaaaccaaaccacgcgaagaacagtataacagtacttatcgggttgtttcagtactcacggtgctccatcaagactggcttaatggaaaggagtataaatgtaaggtaagtaacaaggcattggcggctcccatcgagaagacaatctccaaagcaaaagggcaaccacgggagcctcaggtgtatacgttgccgcccagcagagatgaacttactaagaatcaggtgagtctcacttgtctcgtcaagggcttctatcccagcgatatagccgtagaatgggagagtaacggtcagccggagaacaactacaaaacaaccccgcctgttttggactccgatgggagtttttttctctacagcaaactcacggtagacaaaagcaggtggcagcagggcaatgttttcagttgctctgttctccacgaagccctccactcccactatactcagaagtctctgagtctctcaccagggggaggtagcgatagctggcaggaggaagtgatcaagttgtgcgggcgcgaactcgtgcgggcacaaattgctatatgcggtaaaagtacgggaggtggagaggagggtggaggtgaagaaggcggtggtagacaattgtatagtgcgctcgccaacaagtgttgtcatgtcgggtgtacgaaacggtccttggcgcggttttgc

SEQ ID NO: 489 - DNA, 882 bp, synthetic construct
  source 1..882: mol_type = other DNA
gacaagacacatacttgtccaccatgtcccgccccagaagctgcgggaggaccatcagtttttttgttccccccgaaaccgaaggataccctcatgataagtcgaacgcccgaagtcacttgcgtggtggttgatgttagccacgaggacccagaagtgaagttcaactggtacgtggacggggtcgaagttcataatgcgaaaacaaagcctcgcgaggaacagtacaactctacatacagggttgtgtctgttttgacagtcttgcaccaagattggctcaacgggaaggaatataagtgtaaggtaagcaataaagcactggcggccccgatcgaaaaaacgatatccaaggccaagggccagccccgagagcctcaggtatatactctgccgccaagccgggatgaactgactaaaaaccaggtctctttgacttgtcttgtcaagggattttacccaagtgacattgcggtagagtgggaaagcaacggtcaaccagaaaacaattacaagacgacaccgccggtactcgactcagatggatcctttttcctgtatagcaagctgacagtggacaagtcccggtggcagcaagggaacgtattttcatgcagcgtgatgcatgaggctcttcacaaccattacacacagaaaagtctgtcattgagccctggcggcgggagcgattcttggcaagaagaagttataaaactttgcggtcgagagctggttcgggcacaaattgctatctgcggaaaatctacaggaggaggcgaggagggagggggcgaagaaggcggggggagacagttgtacagtgcgctcgctaacaagtgttgccacgtcggttgcacaaagagatccctggctcaattctgt

SEQ ID NO: 490 - DNA, 882 bp, synthetic construct
  source 1..882: mol_type = other DNA
gataaaactcacacctgtcccccgtgtcccgcaccagaagcggccggtggtccctccgtttttctcttccctcctaaacctaaggacacacttatgattagcagaactccagaagttacgtgcgtagtcgttgacgttagtcatgaagatcctgaggttaagttcaactggtacgtagacggagtagaggtccacaacgccaagacgaaaccccgagaagagcagtataattctacctatcgagttgtttcagtattgacggtgcttcaccaagattggctgaatggcaaagagtataagtgcaaggtaagcaacaaagcactcgcggctcctatcgagaaaactatttccaaagctaagggccagcctcgcgaaccacaagtctataccctgccaccgagtcgggacgaactcaccaagaaccaagtgtctcttacttgcctcgttaaaggtttttatcccagcgacatagccgtcgaatgggagtccaatggccaacctgagaacaactataaaactacccctcctgtacttgatagcgacggaagttttttcctctattcaaaactcacagttgataagtctcgatggcaacagggcaacgtcttctcttgcagtgtgttgcatgaagctctgcactctcattacacacagaagagtttgtctctcagtccaggtggcggctcagatagctggcaggaagaagtaatcaagttgtgcggcagggaactggtaagggcacagatagccatttgtggaaaatctacgggtggcggtgaggaaggcggcggagaagaagggggaggtcggcagctgtatagtgcactcgcaaacaagtgctgccatgtcgggtgcaccaagcgatcccttgcccagttttgc

SEQ ID NO: 491 - DNA, 885 bp, synthetic construct
  source 1..885: mol_type = other DNA
gataagacgcacacatgcccaccctgtcctgcgcctgaagccgcggggggacccagcgtttttctcttcccgccgaaaccgaaagacacacttatgatcagccggactcccgaggttacctgcgtggtggtagatgtatctcacgaggatcccgaggtcaaattcaactggtacgttgatggggttgaagttcataatgccaaaacgaagccaagagaagagcagtataactccacatatagagttgtttccgtcttgactgttcttcaccaagattggctgaatgggaaggagtacaaatgtaaagttagcaacaaggcactcgccgctcccattgaaaaaactataagcaaagctaagggccaaccgcgcgaaccacaggtctacacgttgccgccctctagggacgaactcacgaagaatcaggtttcccttacctgcctcgttaaaggattctacccctctgacatagcggttgaatgggagagcaacggtcagcctgagaacaactacaaaacgacgcctccggtgttggattccgacggtagttttttcctctatagtaagctgacagtggataaatctcggtggcagcaagggaatgtattctcctgttcagtcctgcatgaagccctccactcccattatacacagaaatctctttctctgagtcccggtaaaggtgggagtgactcttggcaggaagaggtaattaagttgtgtggaagggagctggtaagagcacagattgccatctgtggcaaatccacgggcggcgaaggtgaggggggtgagggggaaggggggtccagacaactgtattctgctctggcgaataagtgttgccatgtagggtgcactaaacggtccttggcgcagttctgt

SEQ ID NO: 492 - DNA, 882 bp, synthetic construct
  source 1..882: mol_type = other DNA
gataaaactcatacgtgcccaccttgccccgcaccggaggctgctggaggaccctctgtcttcctgttcccgccgaagcctaaagacacattgatgatcagtcgaacaccggaagtcacctgtgtagtggttgatgtgagccatgaggaccctgaagtaaaatttaactggtatgttgatggcgtagaagtacacaacgcgaagactaaaccaagggaagagcaatacaactctacctatagggtcgttagcgtactgactgtgcttcaccaagactggcttaacgggaaggagtacaagtgcaaagtgagcaataaggccctcgccgcgcctatcgagaaaaccatttccaaagccaagggtcaaccaagggagcctcaggtttacaccctgcccccttcaagggatgagttgacaaaaaaccaggtaagtctgacgtgtctcgttaagggattctacccgtcagatatcgcggtagagtgggagagcaacggtcagccagaaaataattacaaaacaacacctccagttttggactctgatgggagtttttttctttattctaagttgacagtggataagtcacgctggcaacaggggaacgtatttagctgctcagtacttcatgaagcgttgcattctcactacacacagaagagcctctccttgagtcccggaggtggctctgattcttggcaggaggaggtaataaaactttgtggtagagaactggttcgcgctcagatagctatttgtggaaaatccactggcggtgaaggtgaaggtggagaaggagagggcggaagccggcagttgtactctgccctggctaataagtgctgtcacgtgggctgcactaagcggagcttggcaagattttgc

SEQ ID NO: 493 - DNA, 882 bp, synthetic construct
  source 1..882: mol_type = other DNA
gataaaactcatacctgtccaccttgtcctgcgcctgaggcagctggagggcctagcgtgttcctgttcccccccaaacccaaagacacgctcatgattagccgaacccctgaagtgacctgcgttgttgtggacgtaagccacgaagaccccgaagttaagtttaattggtacgtcgacggtgttgaggttcataacgcgaagactaagccgagagaggagcaatataacagcacctaccgcgtagtctcagttcttaccgtgctccaccaggactggcttaacgggaaggaatacaaatgcaaagtttccaacaaagccttggcagccccaatagagaagacaatatctaaggcgaaaggccaaccgcgggaaccgcaagtttataccctcccaccgagcagggatgagctgacaaaaaatcaggtttccctcacttgtctggtcaagggattttatccttcagacatagccgttgaatgggagagtaatgggcagccggagaataattacaagaccacccccccggtgttggacagcgacggttccttctttctctattctaaacttaccgtcgacaaatcacggtggcaacaaggaaatgtattctcatgcagtgtattgcacgaagctctgcactctcattacacccaaaaatccctctctctcagccctggcggtggatctgattcttggcaggaagaggtgattaaactgtgtgggcgagagcttgtccgagctcagatcgctatttgtggcaagagtaccggaggcgagggtgagggaggcgaaggcgagggcggaagccggcaactctatagcgcactcgctaataaatgttgtcatgtcggctgcacgaagcgctcactggcgcagttctgc

SEQ ID NO: 494 - DNA, 36 bp, synthetic construct
  source 1..36: mol_type = other DNA
acgggaccgatccagcctccggactctagagccacc

SEQ ID NO: 495 - DNA, 36 bp, synthetic construct
  source 1..36: mol_type = other DNA
tgataaaccggttagtaatgagtttgatatctcgac

SEQ ID NO: 496 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTASDAAGANANAGARQLYSALANKCCHVGCTKRSLARFC

SEQ ID NO: 497 - protein, 309 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQLYSALANKCCHVGCTKRSLARFCGGGGSGGGGSGGGGSSWMEEVIKLCGRELVRAQIAICGMSTWS

SEQ ID NO: 498 - protein, 329 aa, synthetic construct
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQLYSALANKCCHVGCTKRSLARFCGGGGSGGGGSGGGGSSWMEEVIKLCGRELVRAQIAICGMSTWS

SEQ ID NO: 499 - DNA, 927 bp, synthetic construct
  source 1..927: mol_type = other DNA
gataaaacccatacgtgtcctccatgcccagctcccgagctgctcggtggtccttcagtgttcctcttccccccaaagccgaaggacacgctcatgattagtcgaacgccagaggtgacatgtgtggtcgttgatgtttcccatgaggatccggaagttaagttcaactggtacgtagatggcgtggaggttcacaatgcaaaaaccaagccccgcgaggagcagtataactcaacctacagagtagtatctgtgctcacggtcttgcatcaggattggttgaacgggaaggaatacaagtgtaaagtaagtaataaggcactgccggcccccatagaaaaaactatcagcaaagctaaaggtcagccgcgggagccacaagtttacactcttcctcctagtagagacgagctgacgaagaatcaagtttctttgacttgtctcgtgaagggattctacccaagcgatatagctgtagagtgggaaagcaacggacaaccagaaaataactacaagactacaccccccgttctcgattctgatggctcattcttcttgtactcaaaattgacagttgacaaatctcgatggcagcagggtaacgtatttagttgctctgttatgcacgaagcgttgcataaccactacacacagaagtcattgtcactgagcccaggaaaaggtggtggcgggtccggcggtggaggtagcggtggcgggggctcccagctttatagtgcccttgcaaacaaatgttgccacgtcggatgtacgaagcgcagtttggcgagattctgtggagggggcggatccggaggcggggggtccggaggaggaggtagctcatggatggaagaggtaataaaactgtgcggacgcgagcttgtcagggcccaaatcgcaatttgtggcatgagcacatggagt

SEQ ID NO: 500 - DNA, 987 bp, synthetic construct
  source 1..987: mol_type = other DNA
atggaaactgataccctgttgttgtgggtccttttgctttgggttccaggcagcaccggagataaaacccatacgtgtcctccatgcccagctcccgagctgctcggtggtccttcagtgttcctcttccccccaaagccgaaggacacgctcatgattagtcgaacgccagaggtgacatgtgtggtcgttgatgtttcccatgaggatccggaagttaagttcaactggtacgtagatggcgtggaggttcacaatgcaaaaaccaagccccgcgaggagcagtataactcaacctacagagtagtatctgtgctcacggtcttgcatcaggattggttgaacgggaaggaatacaagtgtaaagtaagtaataaggcactgccggcccccatagaaaaaactatcagcaaagctaaaggtcagccgcgggagccacaagtttacactcttcctcctagtagagacgagctgacgaagaatcaagtttctttgacttgtctcgtgaagggattctacccaagcgatatagctgtagagtgggaaagcaacggacaaccagaaaataactacaagactacaccccccgttctcgattctgatggctcattcttcttgtactcaaaattgacagttgacaaatctcgatggcagcagggtaacgtatttagttgctctgttatgcacgaagcgttgcataaccactacacacagaagtcattgtcactgagcccaggaaaaggtggtggcgggtccggcggtggaggtagcggtggcgggggctcccagctttatagtgcccttgcaaacaaatgttgccacgtcggatgtacgaagcgcagtttggcgagattctgtggagggggcggatccggaggcggggggtccggaggaggaggtagctcatggatggaagaggtaataaaactgtgcggacgcgagcttgtcagggcccaaatcgcaatttgtggcatgagcacatggagt

SEQ ID NO: 501 - protein, 185 aa, synthetic construct
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 502 - protein, 15 aa, synthetic construct
KLCGRELVRAQIAIC

SEQ ID NO: 503 - protein, 16 aa, synthetic construct
KCCHVGCTKRSLARFC

SEQ ID NO: 504 - protein, 16 aa, synthetic construct
KCCHVGCTKRSLAQFC

SEQ ID NO: 505 - protein, 15 aa, synthetic construct
  VARIANT 1: note = K, Q, D, E, L, I or Y
XLCGRELVRAQIAIC

SEQ ID NO: 506 - protein, 16 aa, synthetic construct
  VARIANT 1: note = K, Q, D, E, L, I, or Y
  VARIANT 4: note = any amino acid except for M, W, and C
  VARIANT 9: note = K, Q, D, E, L, I, or Y
  VARIANT 10: note = Q, D, E, L, I, Y or R
  VARIANT 14: note = R, Q, E, D, N, S, or T
XCCXVGCTXXSLAXFC

SEQ ID NO: 507 - protein, 24 aa, synthetic construct
QLYSALANKCCQVGCTKQSLAQFC

SEQ ID NO: 508 - protein, 65 aa, synthetic construct
DSYQEEVIKLCGRELVRAQIAICGKSTGGEGSGGEGEGGGRQLYSALANKCCHVGCTKRSLAQFC

SEQ ID NO: 509 - protein, 65 aa, synthetic construct
DSYQEEVIKLCGRELVRAQIAICGKSTGGEGSGGEGEGGGRQLYSALANKCCQVGCTKRSLAQFC

SEQ ID NO: 510 - protein, 65 aa, synthetic construct
DSYQEEVIKLCGRELVRAQIAICGKSTGGEGSGGEGEGGGRQLYSALANKCCHVGCTKQSLAQFC

SEQ ID NO: 511 - protein, 65 aa, synthetic construct
DSYQEEVIKLCGRELVRAQIAICGKSTGGEGSGGEGEGGGRQLYSALANKCCQVGCTKQSLAQFC

SEQ ID NO: 512 - protein, 65 aa, synthetic construct
DSYQEEVIKLCGRELVRAQIAICGKSTGGEGSGGEGSGGGRQLYSALANKCCHVGCTKRSLAQFC

SEQ ID NO: 513 - protein, 65 aa, synthetic construct
DSYQEEVIKLCGRELVRAQIAICGKSTGGEGSGGEGSGGGRQLYSALANKCCQVGCTKRSLAQFC

SEQ ID NO: 514 - protein, 65 aa, synthetic construct
DSYQEEVIKLCGRELVRAQIAICGKSTGGEGSGGEGSGGGRQLYSALANKCCHVGCTKQSLAQFC

SEQ ID NO: 515 - protein, 65 aa, synthetic construct
DSYQEEVIKLCGRELVRAQIAICGKSTGGEGSGGEGSGGGRQLYSALANKCCQVGCTKQSLAQFC

SEQ ID NO: 516 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCHVGCTKRSLAQFC

SEQ ID NO: 517 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCQVGCTKRSLAQFC

SEQ ID NO: 518 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCHVGCTKQSLAQFC

SEQ ID NO: 519 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCQVGCTKQSLAQFC

SEQ ID NO: 520 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCHVGCTKRSLAQFC

SEQ ID NO: 521 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCQVGCTKRSLAQFC

SEQ ID NO: 522 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCHVGCTKQSLAQFC

SEQ ID NO: 523 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCQVGCTKQSLAQFC

SEQ ID NO: 524 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCHVGCTKRSLAQFC

SEQ ID NO: 525 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCQVGCTKRSLAQFC

SEQ ID NO: 526 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCHVGCTKQSLAQFC

SEQ ID NO: 527 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGEGGGRQLYSALANKCCQVGCTKQSLAQFC

SEQ ID NO: 528 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCHVGCTKRSLAQFC

SEQ ID NO: 529 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCQVGCTKRSLAQFC

SEQ ID NO: 530 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCHVGCTKQSLAQFC

SEQ ID NO: 531 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSYQEEVIKLCGRELVRAQIAICGKSTGGEGSGGEGSGGGRQLYSALANKCCQVGCTKQSLAQFC

SEQ ID NO: 532 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaaggcagctttactctgctctggctaacaagtgttgtcacgttgggtgtaccaagcggtcccttgcgcaattctgc

SEQ ID NO: 533 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaaggcagctttactctgctctggctaacaagtgttgtcaagttgggtgtaccaagcggtcccttgcgcaattctgc

SEQ ID NO: 534 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaaggcagctttactctgctctggctaacaagtgttgtcacgttgggtgtaccaagcaatcccttgcgcaattctgc

SEQ ID NO: 535 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaaggcagctttactctgctctggctaacaagtgttgtcaagttgggtgtaccaagcaatcccttgcgcaattctgc

SEQ ID NO: 536 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaaggcagctttactctgctctggctaacaagtgttgtcacgttgggtgtaccaagcggtcccttgcgcaattctgc

SEQ ID NO: 537 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaaggcagctttactctgctctggctaacaagtgttgtcaagttgggtgtaccaagcggtcccttgcgcaattctgc

SEQ ID NO: 538 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaaggcagctttactctgctctggctaacaagtgttgtcacgttgggtgtaccaagcaatcccttgcgcaattctgc

SEQ ID NO: 539 - DNA, 885 bp, synthetic construct
  source 1..885: mol_type = other DNA
gataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaaggcagctttactctgctctggctaacaagtgttgtcaagttgggtgtaccaagcaatcccttgcgcaattctgc

SEQ ID NO: 540 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaaggcagctttactctgctctggctaacaagtgttgtcacgttgggtgtaccaagcggtcccttgcgcaattctgc

SEQ ID NO: 541 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaaggcagctttactctgctctggctaacaagtgttgtcaagttgggtgtaccaagcggtcccttgcgcaattctgc

SEQ ID NO: 542 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaaggcagctttactctgctctggctaacaagtgttgtcacgttgggtgtaccaagcaatcccttgcgcaattctgc

SEQ ID NO: 543 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggagaagggggcggaaggcagctttactctgctctggctaacaagtgttgtcaagttgggtgtaccaagcaatcccttgcgcaattctgc

SEQ ID NO: 544 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaaggcagctttactctgctctggctaacaagtgttgtcacgttgggtgtaccaagcggtcccttgcgcaattctgc

SEQ ID NO: 545 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaaggcagctttactctgctctggctaacaagtgttgtcaagttgggtgtaccaagcggtcccttgcgcaattctgc

SEQ ID NO: 546 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaaggcagctttactctgctctggctaacaagtgttgtcacgttgggtgtaccaagcaatcccttgcgcaattctgc

SEQ ID NO: 547 - DNA, 945 bp, synthetic construct
  source 1..945: mol_type = other DNA
atggaaacggatacgctgttgttgtgggttctgctcttgtgggtgccagggagcacaggtgataaaacccacacttgcccaccttgccctgcgccggaagccgccggaggacctagtgttttcctctttccccctaagcccaaagacacgttgatgatctctcggacaccggaagtaacttgtgtcgttgtggatgtgtcacatgaggatcccgaggtgaaatttaattggtacgttgacggcgtggaggtgcataacgcaaagactaaaccacgcgaggagcagtataattctacataccgggttgtctcagttctcacagttcttcatcaggattggttgaatggaaaggagtacaaatgcaaagtgtccaacaaagcgcttgctgcgccgattgaaaagacgatttcaaaggcaaaagggcagccccgcgaaccccaagtatatactttgcctccctcacgcgatgaactgactaagaaccaggtgagcctgacttgtttggttaagggtttttatccaagtgacattgctgttgaatgggagtccaatggccagcctgagaataactacaaaacgacacctcctgtacttgacagcgacggctccttttttctttattcaaaactcacagtggacaaatccaggtggcagcagggtaacgtcttttcttgcagcgtgctccacgaagctttgcattcacattatacgcaaaaatccttgtcattgtccccaggtaagggcggaagcgactcataccaagaagaagtcattaaactttgtggacgggagctggttagggcacagattgctatttgtggtaagtctacgggaggagaaggctctggaggcgaaggatctgggggcggaaggcagctttactctgctctggctaacaagtgttgtcaagttgggtgtaccaagcaatcccttgcgcaattctgc

SEQ ID NO: 548 - protein, 19 aa, synthetic construct
MGWSCIILFLVATATGVHS

SEQ ID NO: 549 - protein, 314 aa, synthetic construct
MGWSCIILFLVATATGVHSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTASDAAGAEAEAGARQLYSALANKCCHVGCTKRSLAQFC

SEQ ID NO: 550 - protein, 24 aa, synthetic construct
QLYSALANKCCHVGCTKRSLAEFC

SEQ ID NO: 551 - protein, 25 aa, synthetic construct
RQLYSALANKCCHVGCTKQSLAQFC

SEQ ID NO: 552 - protein, 25 aa, synthetic construct
RQLYSALANKCCYVGCTKRSLAQFC

SEQ ID NO: 553 - protein, 25 aa, synthetic construct
RQLYSALANKCCLVGCTKRSLAQFC

SEQ ID NO: 554 - protein, 25 aa, synthetic construct
RQLYSALANKCCQVGCTKRSLAQFC

SEQ ID NO: 555 - protein, 25 aa, synthetic construct
RQLYSALANKCCKVGCTKRSLAQFC

SEQ ID NO: 556 - protein, 25 aa, synthetic construct
RQLYSALANKCCYVGCTKQSLAQFC

SEQ ID NO: 557 - protein, 25 aa, synthetic construct
RQLYSALANKCCKVGCTKQSLAQFC

SEQ ID NO: 558 - protein, 25 aa, synthetic construct
RQLYSALANKCCQVGCTKQSLAQFC

(19) United States
(12) Patent Application Publication
(10) Pub. No.: US 2025/0333467 A1
(43) Pub. Date: Oct. 30, 2025

(54) RELAXIN-2 FUSION PROTEIN ANALOGS AND METHODS OF USING SAME

(71) Applicant: TECTONIC OPERATING COMPANY, INC., Watertown, MA (US)

(72) Inventors: John Diener, Watertown, MA (US); Ryan Knihtila, Watertown, MA (US); Washington Alves, Watertown, MA (US); Anthony J. Muslin, Watertown, MA (US); Franz Gruswitz, Watertown, MA (US)

(21) Appl. No.: 19/063,612

(22) Filed: Feb. 26, 2025

Related U.S. Application Data
(62) Division of application No. 18/799,714, filed on Aug. 9, 2024, now Pat. No. 12,264,188.
Application No. 18/799,714 is a continuation of application No. PCT/US2024/030016, filed on May 17, 2024.
(60) Provisional application No. 63/503,101, filed on May 18, 2023.
(60) Provisional application No. 63/585,849, filed on Sep. 27, 2023.
(60) Provisional application No. 63/586,868, filed on Sep. 29, 2023.
(60) Provisional application No. 63/611,732, filed on Dec. 18, 2023.
(60) Provisional application No. 63/617,398, filed on Jan. 3, 2024.

(51) Int. Cl. C07K 14/64; A61K 38/00; A61P 43/00

(52) U.S. Cl. CPC C07K 14/64; A61P 43/00; A61K 38/00; C07K 2319/30

(57) ABSTRACT

Relaxin-2 fusion protein analogs with enhanced in vivo half-life and methods for making the same are described. Also described are methods of treating relaxin-2-associated disorders or diseases using the relaxin-2 fusion protein analogs.

### RELATED APPLICATIONS

This application is a division of U.S. patent application Ser. No. 18/799,714, filed Aug. 9, 2024, which is a continuation of PCT/US2024/030016, filed May 17, 2024, which claims priority to U.S. Provisional Patent Application Ser. Nos. 63/503,101, filed May 18, 2023, 63/585,849, filed Sep. 27, 2023, 63/586,868, filed Sep. 29, 2023, 63/611,732, filed Dec. 18, 2023, and 63/617,398, filed Jan. 3, 2024, the entire disclosures of which are hereby incorporated by reference herein.

### REFERENCE TO SEQUENCE LISTING

This application contains a sequence listing which has been submitted electronically in XML format and is hereby incorporated by reference in its entirety (said XML copy, created Feb. 25, 2025, is named “216481_seqlist.xml” and is 728,179 bytes in size).

### BACKGROUND

Relaxin-2 exhibits strong antifibrotic activity. In injured tissues, fibroblast activation and proliferation cause increased collagen production and interstitial fibrosis. Fibrosis in the heart is increased by biomechanical overload, and influences ventricular dysfunction, remodeling, and arrhythmogenesis. However, due to the limited in vivo half-life of relaxin, compound administration has to be performed as a continuous infusion for at least 48 hours. Further, the synthesis of relaxin-2 is difficult. Due to the low solubility of the B-chain and the requirement for the laborious, specific introduction of cysteine bridges between the A and B-chains, yields of active peptide obtained by these methods are extremely low.

There is a need for an engineered relaxin-2 analog with greater half-life and greater ease in production.

### SUMMARY

This disclosure provides fusion proteins that are engineered relaxin-2 analogs with improved pharmacokinetic properties. This disclosure also provides methods of using these fusion proteins to enhance relaxin-2 related activity in a subject and to treat or prevent relaxin-2 related diseases. The structure of the fusion proteins described herein is based, at least in part, upon the surprising discovery that reducing the isoelectric point (pI) of relaxin-2 fusion protein analogs increases their circulating half-life and improves their pharmacokinetic and pharmacodynamic properties.

Accordingly, in one aspect, the present disclosure provides a fusion protein comprising, from N-terminus to C-terminus, a first peptide; a linker peptide; and a second peptide, wherein: (a) the first peptide comprises an amino acid sequence that has 0, 1, 2, 3, 4, or 5 amino acid modifications relative to the amino acid sequence of SEQ ID NO: 502 and the second peptide comprises an amino acid sequence that that has 0, 1, 2, 3, 4, or 5 amino acid modifications relative to the amino acid sequence of SEQ ID NO: 503 or 504; or the first peptide comprises an amino acid sequence that has 0, 1, 2, 3, 4, or 5 amino acid modifications relative to the amino acid sequence of SEQ ID NO: 503 or 504 and the second peptide comprises an amino acid sequence that that has 0, 1, 2, 3, 4, or 5 amino acid modifications relative to the amino acid sequence of SEQ ID NO: 502; and optionally, (b) the fusion protein has a pI from 6.0 to 8.2.

In some embodiments, the fusion protein has a pI from about 6.0 to about 9.4. In some embodiments, the fusion protein has a pI from about 6.0 to about 8.2. In some embodiments, the fusion protein has a pI that is less than about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, or 6.1. In some embodiments, the fusion protein has a pI that is less than 9.0. In some embodiments, the fusion protein has a pI that is less than about 8.2. In some embodiments, the fusion protein has a pI of about 6.8. In some embodiments, the fusion protein has a pI of about 7.0. In some embodiments, the fusion protein has a pI of about 7.1. In some embodiments, the fusion protein has a pI of about 7.4. In some embodiments, the fusion protein has a pI of about 7.5. In some embodiments, the fusion protein has a pI of about 7.9. In some embodiments, the fusion protein has a pI of about 8.0. In some embodiments, the fusion protein has a pI of about 8.4. In some embodiments, the fusion protein has a pI of about 8.5. In some embodiments, the fusion protein has a pI of about 8.8. In some embodiments, the fusion protein has a pI of about 8.9.

In some embodiments, the first peptide comprises the amino acid sequence X₁₁LCGRELVRAQIAIC (SEQ ID NO: 505), wherein X₁₁ is K, Q, D, E, L, I or Y. In some embodiments, the first peptide consists of 15, 16, 17, 18, 19, 20, 21, 22, 23, 24, 25, 26, 27, 28, or 29 amino acids.

In some embodiments, the first peptide comprises the amino acid sequence X₁₂CCX₁₃VGCTX₁₄X₁₅SLAX₁₆FC (SEQ ID NO: 506), wherein: X₁₂ is K, Q, D, E, L, I, or Y; X₁₃ is any amino acid except M, W, or C; X₁₄ is K, Q, D, E, L, I, or Y; X₁₅ is Q, D, E, L, I, Y or R; and X₁₆ is R or Q. In some embodiments, the first peptide comprises the amino acid sequence X₁₂CCX₁₃VGCTX₁₄X₁₅SLAX₁₆FC (SEQ ID NO: 506), wherein: X₁₂ is K, Q, D, E, L, I, or Y; X₁₃ is H, K, Q, Y, L, N, I, S, T, or F; X₁₄ is K, Q, D, E, L, I, or Y; X₁₅ is Q, D, E, L, I, Y or R; and X₁₆ is R or Q. In some embodiments, X₁₃ is Q. In some embodiments, the first peptide consists of 16, 17, 18, 19, 20, 21, 22, 23, 24, or 25 amino acids.

In some embodiments, the second peptide comprises the amino acid sequence X₁₁LCGRELVRAQIAIC (SEQ ID NO: 505), wherein X₁₁ is K, Q, D, E, L, I or Y. In some embodiments, the second peptide consists of 15, 16, 17, 18, 19, 20, 21, 22, 23, 24, 25, 26, 27, 28, or 29 amino acids.

In some embodiments, the second peptide comprises the amino acid sequence X₁₂CCX₁₃VGCTX₁₄X₁₅SLAX₁₆FC (SEQ ID NO: 506), wherein: X₁₂ is K, Q, D, E, L, I, or Y; X₁₃ is any amino acid except M, W, or C; X₁₄ is K, Q, D, E, L, I, or Y; X₁₅ is Q, D, E, L, I, Y or R; and X₁₆ is R or Q. In some embodiments, the second peptide comprises the amino acid sequence X₁₂CCX₁₃VGCTX₁₄X₁₅SLAX₁₆FC (SEQ ID NO: 506), wherein: X₁₂ is K, Q, D, E, L, I, or Y; X₁₃ is H, K, Q, Y, L, N, I, S, T, or F; X₁₄ is K, Q, D, E, L, I, or Y; X₁₅ is Q, D, E, L, I, Y or R; and X₁₆ is R or Q. In some embodiments, X₁₃ is Q. In some embodiments, the second peptide consists of 16, 17, 18, 19, 20, 21, 22, 23, 24, or 25 amino acids.

In some embodiments, the linker peptide comprises an amino acid sequence with 12-15 amino acids. In some embodiments, the linker peptide comprises the amino acid sequence ASDAAGAX₈AX₉AGA (SEQ ID NO: 17), wherein: X₈ is D, E, N, or Q; and X₉ is D, E, N, or Q; or the linker peptide comprises the amino acid sequence GGEGSGGEGX₁₀GGG (SEQ ID NO: 25), wherein: X₁₀ is E or S. In some embodiments, X₈ is D, E, N, or Q, and X₉ is D, E, or Q; or X₈ is D, E, or Q, and X₉ is D, E, N, or Q. In some embodiments, the linker peptide comprises an amino acid sequence selected from the group consisting of SEQ ID NOs: 18, 19, 20, 21, 22, 23, 24, 26, and 27.

In another aspect, the present disclosure provides a fusion protein comprising, from N-terminus to C-terminus, a first peptide; a linker peptide; and a second peptide, wherein: (a) the first peptide comprises an amino acid sequence that has 0, 1, 2, 3, 4, or 5 amino acid modifications relative to the amino acid sequence of SEQ ID NO: 1, wherein the amino acid at at least one of positions 4 or 25 of the first peptide is not M; and the second peptide comprises an amino acid sequence that has 0, 1, 2, 3, 4, or 5 amino acid modifications relative to the amino acid sequence of SEQ ID NO: 8, wherein the amino acid at position 22 of the second peptide is not R; or the first peptide comprises an amino acid sequence that has 0, 1, 2, 3, 4, or 5 amino acid modifications relative to the amino acid sequence of SEQ ID NO: 8, wherein the amino acid at position 22 of the second peptide is not R; and the second peptide comprises an amino acid sequence that has 0, 1, 2, 3, 4, or 5 amino acid modifications relative to the amino acid sequence of SEQ ID NO: 1, wherein the amino acid at at least one of positions 4 or 25 of the first peptide is not M; and optionally, (b) the fusion protein has a pI from 6.0 to 8.2.

In some embodiments, the fusion protein has a pI from about 6.0 to about 9.4. In some embodiments, the fusion protein has a pI from about 6.0 to about 8.2. In some embodiments, the fusion protein has a pI that is less than about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, or 6.1. In some embodiments, the fusion protein has a pI that is less than 9.0. In some embodiments, the fusion protein has a pI that is less than about 8.2. In some embodiments, the fusion protein has a pI of about 6.8. In some embodiments, the fusion protein has a pI of about 7.0. In some embodiments, the fusion protein has a pI of about 7.1. In some embodiments, the fusion protein has a pI of about 7.4. In some embodiments, the fusion protein has a pI of about 7.5. In some embodiments, the fusion protein has a pI of about 7.9. In some embodiments, the fusion protein has a pI of about 8.0. In some embodiments, the fusion protein has a pI of about 8.4. In some embodiments, the fusion protein has a pI of about 8.5. In some embodiments, the fusion protein has a pI of about 8.8. In some embodiments, the fusion protein has a pI of about 8.9.

In some embodiments, the linker peptide comprises an amino acid sequence with 12-15 amino acids. In some embodiments, the linker peptide comprises the amino acid sequence ASDAAGAX₈AX₉AGA (SEQ ID NO: 17), wherein: X₈ is D, E, N, or Q; and X₉ is D, E, N, or Q; or the linker peptide comprises the amino acid sequence GGEGSGGEGX₁₀GGG (SEQ ID NO: 25), wherein: X₁₀ is E or S. In some embodiments, X₈ is D, E, N, or Q, and X₉ is D, E, or Q; or X₈ is D, E, or Q, and X₉ is D, E, N, or Q.

In another aspect, the present disclosure provides a fusion protein comprising, from N-terminus to C-terminus: a first peptide; a linker peptide; and a second peptide, wherein: the linker peptide comprises the amino acid sequence ASDAAGAX₈AX₉AGA (SEQ ID NO: 17), wherein: X₈ is D, E, N, or Q; and X₉ is D, E, N, or Q; or the linker peptide comprises the amino acid sequence GGEGSGGEGX₁₀GGG (SEQ ID NO: 25), wherein: X₁₀ is E or S.

In some embodiments, X₈ is D, E, N, or Q, and X₉ is D, E, or Q; or X₈ is D, E, or Q, and X₉ is D, E, N, or Q. In some embodiments, the linker peptide comprises an amino acid sequence selected from the group consisting of SEQ ID NOs: 18, 19, 20, 21, 22, 23, 24, 26, and 27.

In some embodiments, the first peptide comprises the amino acid sequence DSX₁QEEVIX₂LCGRELVRAQIAICGX₃ST (SEQ ID NO: 7), wherein: X₁ is not M, H, or C; X₂ is K, Q, D, E, L, I or Y; and X₃ is K or Q. In some embodiments, the first peptide comprises the amino acid sequence DSX₁QEEVIX₂LCGRELVRAQIAICGX₃ST (SEQ ID NO: 7), wherein: X₁ is W, Y, F, L, I, V or A; X₂ is K, Q, D, E, L, I or Y; and X₃ is K or Q. In some embodiments, X₁ is Y. In some embodiments, the first peptide comprises an amino acid sequence selected from the group consisting of SEQ ID NOs: 1, 2, 3, 4, 5, and 6. In some embodiments, the first peptide consists of 27, 28, or 29 amino acids.

In some embodiments, the first peptide comprises the amino acid sequence QLYSALANX₄CCX₅VGCTX₆X₇SLAQFC (SEQ ID NO: 16), wherein: X₄ is K, Q, D, E, L, I, or Y; X₅ is any amino acid except M, W, or C; X₆ is K, Q, D, E, L, I, or Y; and X₇ is Q, D, E, L, I, Y or R. In some embodiments, the first peptide comprises the amino acid sequence QLYSALANX₄CCX₅VGCTX₆X₇SLAQFC (SEQ ID NO: 16), wherein: X₄ is K, Q, D, E, L, I, or Y; X₅ is H, K, Q, Y, L, N, I, S, T, or F; X₆ is K, Q, D, E, L, I, or Y; and X₇ is Q, D, E, L, I, Y or R. In some embodiments, X₅ is Q. In some embodiments, the first peptide comprises an amino acid sequence selected from the group consisting of SEQ ID NOs: 8, 9, 10, 11, 12, 13, 14, 15, and 507. In some embodiments, the first peptide consists of 24 or 25 amino acids.

In some embodiments, the second peptide comprises the amino acid sequence DSX₁QEEVIX₂LCGRELVRAQIAICGX₃ST (SEQ ID NO: 7), wherein: X₁ is not M, H, or C; X₂ is K, Q, D, E, L, I or Y; and X₃ is K or Q. In some embodiments, the second peptide comprises the amino acid sequence DSX₁QEEVIX₂LCGRELVRAQIAICGX₃ST (SEQ ID NO: 7), wherein: X₁ is W, Y, F, L, I, V or A; X₂ is K, Q, D, E, L, I, or Y; and X₃ is K or Q. In some embodiments, X₁ is Y. In some embodiments, the second peptide comprises an amino acid sequence selected from the group consisting of SEQ ID NOs: 1, 2, 3, 4, 5, and 6. In some embodiments, the second peptide consists of 27, 28, or 29 amino acids.

In some embodiments, the second peptide comprises the amino acid sequence QLYSALANX₄CCX₅VGCTX₆X₇SLAQFC (SEQ ID NO: 16), wherein: X₄ is K, Q, D, E, L, I, or Y; X₅ is any amino acid except M, W, or C; X₆ is K, Q, D, E, L, I, or Y; and X₇ is Q, D, E, L, I, Y or R. In some embodiments, the second peptide comprises the amino acid sequence QLYSALANX₄CCX₅VGCTX₆X-SLAQFC (SEQ ID NO: 16), wherein: X₄ is K, Q, D, E, L, I, or Y; X₅ is H, K, Q, Y, L, N, I, S, T, or F; X₆ is K, Q, D, E, L, I, or Y; and X₇ is Q, D, E, L, I, Y or R. In some embodiments, X₅ is Q. In some embodiments, the second peptide comprises an amino acid sequence selected from the group consisting of SEQ ID NOs: 8, 9, 10, 11, 12, 13, 14, 15, and 507. In some embodiments, the second peptide consists of 24 or 25 amino acids.

In some embodiments, the first peptide comprises the amino acid sequence of SEQ ID NO: 1 and the second peptide comprises the amino acid sequence of SEQ ID NO: 8; the first peptide comprises the amino acid sequence of SEQ ID NO: 1 and the second peptide comprises the amino acid sequence of SEQ ID NO: 9; the first peptide comprises the amino acid sequence of SEQ ID NO: 1 and the second peptide comprises the amino acid sequence of SEQ ID NO: 10; the first peptide comprises the amino acid sequence of SEQ ID NO: 1 and the second peptide comprises the amino acid sequence of SEQ ID NO: 11; the first peptide comprises the amino acid sequence of SEQ ID NO: 1 and the second peptide comprises the amino acid sequence of SEQ ID NO: 12; the first peptide comprises the amino acid sequence of SEQ ID NO: 1 and the second peptide comprises the amino acid sequence of SEQ ID NO: 13; the first peptide comprises the amino acid sequence of SEQ ID NO: 1 and the second peptide comprises the amino acid sequence of SEQ ID NO: 14; the first peptide comprises the amino acid sequence of SEQ ID NO: 1 and the second peptide comprises the amino acid sequence of SEQ ID NO: 15; the first peptide comprises the amino acid sequence of SEQ ID NO: 1 and the second peptide comprises the amino acid sequence of SEQ ID NO: 507; the first peptide comprises the amino acid sequence of SEQ ID NO: 2 and the second peptide comprises the amino acid sequence of SEQ ID NO: 8; the first peptide comprises the amino acid sequence of SEQ ID NO: 2 and the second peptide comprises the amino acid sequence of SEQ ID NO: 9; the first peptide comprises the amino acid sequence of SEQ ID NO: 2 and the second peptide comprises the amino acid sequence of SEQ ID NO: 10; the first peptide comprises the amino acid sequence of SEQ ID NO: 2 and the second peptide comprises the amino acid sequence of SEQ ID NO: 11; the first peptide comprises the amino acid sequence of SEQ ID NO: 2 and the second peptide comprises the amino acid sequence of SEQ ID NO: 12; the first peptide comprises the amino acid sequence of SEQ ID NO: 2 and the second peptide comprises the amino acid sequence of SEQ ID NO: 13; the first peptide comprises the amino acid sequence of SEQ ID NO: 2 and the second peptide comprises the amino acid sequence of SEQ ID NO: 14; the first peptide comprises the amino acid sequence of SEQ ID NO: 2 and the second peptide comprises the amino acid sequence of SEQ ID NO: 15; the first peptide comprises the amino acid sequence of SEQ ID NO: 2 and the second peptide comprises the amino acid sequence of SEQ ID NO: 507; the first peptide comprises the amino acid sequence of SEQ ID NO: 3 and the second peptide comprises the amino acid sequence of SEQ ID NO: 8; the first peptide comprises the amino acid sequence of SEQ ID NO: 3 and the second peptide comprises the amino acid sequence of SEQ ID NO: 9; the first peptide comprises the amino acid sequence of SEQ ID NO: 3 and the second peptide comprises the amino acid sequence of SEQ ID NO: 10; the first peptide comprises the amino acid sequence of SEQ ID NO: 3 and the second peptide comprises the amino acid sequence of SEQ ID NO: 11; the first peptide comprises the amino acid sequence of SEQ ID NO: 3 and the second peptide comprises the amino acid sequence of SEQ ID NO: 12; the first peptide comprises the amino acid sequence of SEQ ID NO: 3 and the second peptide comprises the amino acid sequence of SEQ ID NO: 13; the first peptide comprises the amino acid sequence of SEQ ID NO: 3 and the second peptide comprises the amino acid sequence of SEQ ID NO: 14; the first peptide comprises the amino acid sequence of SEQ ID NO: 3 and the second peptide comprises the amino acid sequence of SEQ ID NO: 15; the first peptide comprises the amino acid sequence of SEQ ID NO: 3 and the second peptide comprises the amino acid sequence of SEQ ID NO: 507; the first peptide comprises the amino acid sequence of SEQ ID NO: 4 and the second peptide comprises the amino acid sequence of SEQ ID NO: 8; the first peptide comprises the amino acid sequence of SEQ ID NO: 4 and the second peptide comprises the amino acid sequence of SEQ ID NO: 9; the first peptide comprises the amino acid sequence of SEQ ID NO: 4 and the second peptide comprises the amino acid sequence of SEQ ID NO: 10; the first peptide comprises the amino acid sequence of SEQ ID NO: 4 and the second peptide comprises the amino acid sequence of SEQ ID NO: 11; the first peptide comprises the amino acid sequence of SEQ ID NO: 4 and the second peptide comprises the amino acid sequence of SEQ ID NO: 12; the first peptide comprises the amino acid sequence of SEQ ID NO: 4 and the second peptide comprises the amino acid sequence of SEQ ID NO: 13; the first peptide comprises the amino acid sequence of SEQ ID NO: 4 and the second peptide comprises the amino acid sequence of SEQ ID NO: 14; the first peptide comprises the amino acid sequence of SEQ ID NO: 4 and the second peptide comprises the amino acid sequence of SEQ ID NO: 15; the first peptide comprises the amino acid sequence of SEQ ID NO: 4 and the second peptide comprises the amino acid sequence of SEQ ID NO: 507; the first peptide comprises the amino acid sequence of SEQ ID NO: 5 and the second peptide comprises the amino acid sequence of SEQ ID NO: 8; the first peptide comprises the amino acid sequence of SEQ ID NO: 5 and the second peptide comprises the amino acid sequence of SEQ ID NO: 9; the first peptide comprises the amino acid sequence of SEQ ID NO: 5 and the second peptide comprises the amino acid sequence of SEQ ID NO: 10; the first peptide comprises the amino acid sequence of SEQ ID NO: 5 and the second peptide comprises the amino acid sequence of SEQ ID NO: 11; the first peptide comprises the amino acid sequence of SEQ ID NO: 5 and the second peptide comprises the amino acid sequence of SEQ ID NO: 12; the first peptide comprises the amino acid sequence of SEQ ID NO: 5 and the second peptide comprises the amino acid sequence of SEQ ID NO: 13; the first peptide comprises the amino acid sequence of SEQ ID NO: 5 and the second peptide comprises the amino acid sequence of SEQ ID NO: 14; the first peptide comprises the amino acid sequence of SEQ ID NO: 5 and the second peptide comprises the amino acid sequence of SEQ ID NO: 15; the first peptide comprises the amino acid sequence of SEQ ID NO: 5 and the second peptide comprises the amino acid sequence of SEQ ID NO: 507; the first peptide comprises the amino acid sequence of SEQ ID NO: 6 and the second peptide comprises the amino acid sequence of SEQ ID NO: 8; the first peptide comprises the amino acid sequence of SEQ ID NO: 6 and the second peptide comprises the amino acid sequence of SEQ ID NO: 9; the first peptide comprises the amino acid sequence of SEQ ID NO: 6 and the second peptide comprises the amino acid sequence of SEQ ID NO: 10; the first peptide comprises the amino acid sequence of SEQ ID NO: 6 and the second peptide comprises the amino acid sequence of SEQ ID NO: 11; the first peptide comprises the amino acid sequence of SEQ ID NO: 6 and the second peptide comprises the amino acid sequence of SEQ ID NO: 12; the first peptide comprises the amino acid sequence of SEQ ID NO: 6 and the second peptide comprises the amino acid sequence of SEQ ID NO: 13; the first peptide comprises the amino acid sequence of SEQ ID NO: 6 and the second peptide comprises the amino acid sequence of SEQ ID NO: 14; the first peptide comprises the amino acid sequence of SEQ ID NO: 6 and the second peptide comprises the amino acid sequence of SEQ ID NO: 15; or the first peptide comprises the amino acid sequence of SEQ ID NO: 6 and the second peptide comprises the amino acid sequence of SEQ ID NO: 507.

In some embodiments, the first peptide comprises the amino acid sequence of SEQ ID NO: 8 and the second peptide comprises the amino acid sequence of SEQ ID NO: 1; the first peptide comprises the amino acid sequence of SEQ ID NO: 8 and the second peptide comprises the amino acid sequence of SEQ ID NO: 2; the first peptide comprises the amino acid sequence of SEQ ID NO: 8 and the second peptide comprises the amino acid sequence of SEQ ID NO: 3; the first peptide comprises the amino acid sequence of SEQ ID NO: 8 and the second peptide comprises the amino acid sequence of SEQ ID NO: 4; the first peptide comprises the amino acid sequence of SEQ ID NO: 8 and the second peptide comprises the amino acid sequence of SEQ ID NO: 5; the first peptide comprises the amino acid sequence of SEQ ID NO: 8 and the second peptide comprises the amino acid sequence of SEQ ID NO: 6; the first peptide comprises the amino acid sequence of SEQ ID NO: 9 and the second peptide comprises the amino acid sequence of SEQ ID NO: 1; the first peptide comprises the amino acid sequence of SEQ ID NO: 9 and the second peptide comprises the amino acid sequence of SEQ ID NO: 2; the first peptide comprises the amino acid sequence of SEQ ID NO: 9 and the second peptide comprises the amino acid sequence of SEQ ID NO: 3; the first peptide comprises the amino acid sequence of SEQ ID NO: 9 and the second peptide comprises the amino acid sequence of SEQ ID NO: 4; the first peptide comprises the amino acid sequence of SEQ ID NO: 9 and the second peptide comprises the amino acid sequence of SEQ ID NO: 5; the first peptide comprises the amino acid sequence of SEQ ID NO: 9 and the second peptide comprises the amino acid sequence of SEQ ID NO: 6; the first peptide comprises the amino acid sequence of SEQ ID NO: 10 and the second peptide comprises the amino acid sequence of SEQ ID NO: 1; the first peptide comprises the amino acid sequence of SEQ ID NO: 10 and the second peptide comprises the amino acid sequence of SEQ ID NO: 2; the first peptide comprises the amino acid sequence of SEQ ID NO: 10 and the second peptide comprises the amino acid sequence of SEQ ID NO: 3; the first peptide comprises the amino acid sequence of SEQ ID NO: 10 and the second peptide comprises the amino acid sequence of SEQ ID NO: 4; the first peptide comprises the amino acid sequence of SEQ ID NO: 10 and the second peptide comprises the amino acid sequence of SEQ ID NO: 5; the first peptide comprises the amino acid sequence of SEQ ID NO: 10 and the second peptide comprises the amino acid sequence of SEQ ID NO: 6; the first peptide comprises the amino acid sequence of SEQ ID NO: 11 and the second peptide comprises the amino acid sequence of SEQ ID NO: 1; the first peptide comprises the amino acid sequence of SEQ ID NO: 11 and the second peptide comprises the amino acid sequence of SEQ ID NO: 2; the first peptide comprises the amino acid sequence of SEQ ID NO: 11 and the second peptide comprises the amino acid sequence of SEQ ID NO: 3; the first peptide comprises the amino acid sequence of SEQ ID NO: 11 and the second peptide comprises the amino acid sequence of SEQ ID NO: 4; the first peptide comprises the amino acid sequence of SEQ ID NO: 11 and the second peptide comprises the amino acid sequence of SEQ ID NO: 5; the first peptide comprises the amino acid sequence of SEQ ID NO: 11 and the second peptide comprises the amino acid sequence of SEQ ID NO: 6; the first peptide comprises the amino acid sequence of SEQ ID NO: 12 and the second peptide comprises the amino acid sequence of SEQ ID NO: 1; the first peptide comprises the amino acid sequence of SEQ ID NO: 12 and the second peptide comprises the amino acid sequence of SEQ ID NO: 2; the first peptide comprises the amino acid sequence of SEQ ID NO: 12 and the second peptide comprises the amino acid sequence of SEQ ID NO: 3; the first peptide comprises the amino acid sequence of SEQ ID NO: 12 and the second peptide comprises the amino acid sequence of SEQ ID NO: 4; the first peptide comprises the amino acid sequence of SEQ ID NO: 12 and the second peptide comprises the amino acid sequence of SEQ ID NO: 5; the first peptide comprises the amino acid sequence of SEQ ID NO: 12 and the second peptide comprises the amino acid sequence of SEQ ID NO: 6; the first peptide comprises the amino acid sequence of SEQ ID NO: 13 and the second peptide comprises the amino acid sequence of SEQ ID NO: 1; the first peptide comprises the amino acid sequence of SEQ ID NO: 13 and the second peptide comprises the amino acid sequence of SEQ ID NO: 2; the first peptide comprises the amino acid sequence of SEQ ID NO: 13 and the second peptide comprises the amino acid sequence of SEQ ID NO: 3; the first peptide comprises the amino acid sequence of SEQ ID NO: 13 and the second peptide comprises the amino acid sequence of SEQ ID NO: 4; the first peptide comprises the amino acid sequence of SEQ ID NO: 13 and the second peptide comprises the amino acid sequence of SEQ ID NO: 5; the first peptide comprises the amino acid sequence of SEQ ID NO: 13 and the second peptide comprises the amino acid sequence of SEQ ID NO: 6; the first peptide comprises the amino acid sequence of SEQ ID NO: 14 and the second peptide comprises the amino acid sequence of SEQ ID NO: 1; the first peptide comprises the amino acid sequence of SEQ ID NO: 14 and the second peptide comprises the amino acid sequence of SEQ ID NO: 2; the first peptide comprises the amino acid sequence of SEQ ID NO: 14 and the second peptide comprises the amino acid sequence of SEQ ID NO: 3; the first peptide comprises the amino acid sequence of SEQ ID NO: 14 and the second peptide comprises the amino acid sequence of SEQ ID NO: 4; the first peptide comprises the amino acid sequence of SEQ ID NO: 14 and the second peptide comprises the amino acid sequence of SEQ ID NO: 5; the first peptide comprises the amino acid sequence of SEQ ID NO: 14 and the second peptide comprises the amino acid sequence of SEQ ID NO: 6; the first peptide comprises the amino acid sequence of SEQ ID NO: 15 and the second peptide comprises the amino acid sequence of SEQ ID NO: 1; the first peptide comprises the amino acid sequence of SEQ ID NO: 15 and the second peptide comprises the amino acid sequence of SEQ ID NO: 2; the first peptide comprises the amino acid sequence of SEQ ID NO: 15 and the second peptide comprises the amino acid sequence of SEQ ID NO: 3; the first peptide comprises the amino acid sequence of SEQ ID NO: 15 and the second peptide comprises the amino acid sequence of SEQ ID NO: 4; the first peptide comprises the amino acid sequence of SEQ ID NO: 15 and the second peptide comprises the amino acid sequence of SEQ ID NO: 5; the first peptide comprises the amino acid sequence of SEQ ID NO: 15 and the second peptide comprises the amino acid sequence of SEQ ID NO: 6; the first peptide comprises the amino acid sequence of SEQ ID NO: 507 and the second peptide comprises the amino acid sequence of SEQ ID NO: 1; the first peptide comprises the amino acid sequence of SEQ ID NO: 507 and the second peptide comprises the amino acid sequence of SEQ ID NO: 2; the first peptide comprises the amino acid sequence of SEQ ID NO: 507 and the second peptide comprises the amino acid sequence of SEQ ID NO: 3; the first peptide comprises the amino acid sequence of SEQ ID NO: 507 and the second peptide comprises the amino acid sequence of SEQ ID NO: 4; the first peptide comprises the amino acid sequence of SEQ ID NO: 507 and the second peptide comprises the amino acid sequence of SEQ ID NO: 5; or the first peptide comprises the amino acid sequence of SEQ ID NO: 507 and the second peptide comprises the amino acid sequence of SEQ ID NO: 6.

In some embodiments, the fusion protein comprises an amino acid sequence selected from the group consisting of SEQ ID NOs: 28-75 and 508-515.

In some embodiments, the fusion protein further comprises an IgG Fc. In some embodiments, the IgG Fc comprises the amino acid alanine at each of EU positions 234 and 235. In some embodiments, the IgG Fc comprises the amino acid alanine at EU position 329. In some embodiments, the IgG Fc comprises the amino acid alanine at each of EU positions 234, 235, and 329. In some embodiments, the IgG Fc comprises the amino acids alanine, alanine, alanine, leucine, and serine at EU positions 234, 235, 329, 428, and 434, respectively. In some embodiments, the IgG Fc comprises the amino acids lysine, phenylalanine, and tyrosine at EU positions 433, 434, and 436, respectively. In some embodiments, the IgG Fc comprises the amino acids tyrosine, threonine, and glutamate at EU positions 252, 254, and 256, respectively. In some embodiments, the IgG Fc comprises the amino acids leucine and serine at EU positions 428 and 434, respectively.

In some embodiments, the IgG Fc comprises an amino acid sequence at least 85% identical to the amino acid sequence of a human IgG1 Fc. In some embodiments, the IgG Fc comprises the amino acid sequence of a human IgG1 Fc.

In some embodiments, the IgG Fc comprises an amino acid sequence at least 95% identical to an amino acid sequence selected from the group consisting of SEQ ID NOs: 76-83. In some embodiments, the IgG Fc comprises an amino acid sequence selected from the group consisting of SEQ ID NOs: 76-83.

In some embodiments, the IgG Fc is linked to the N-terminus of the first peptide. In some embodiments, the IgG Fc is linked to the C-terminus of the second peptide.

In some embodiments, the fusion protein comprises an amino acid sequence selected from the group consisting of SEQ ID NOs: 84-138 and 516-523. In some embodiments, the fusion protein comprises an amino acid sequence selected from the group consisting of SEQ ID NOs: 139-193, 524-531, and 549.

In another aspect, the present disclosure provides a polypeptide comprising an amino acid sequence selected from the group consisting of SEQ ID NOs: 1-6, 8-15, 18-24, 26-75, 84-193, 507-531, and 549-558.

In another aspect, the present disclosure provides a polynucleotide comprising a nucleotide sequence encoding any one of the fusion proteins described herein, or any one of the polypeptides described herein.

In some embodiments, the polynucleotide is a DNA molecule. In some embodiments, the polynucleotide comprises a nucleotide sequence selected from the group consisting of SEQ ID NOs: 194-248, 410-464, and 532-547.

In some embodiments, the polynucleotide is an RNA molecule.

In another aspect, the present disclosure provides an expression vector comprising the any one of the polynucleotides described herein.

In some embodiments, the expression vector is a plasmid. In some embodiments, the expression vector is a viral vector.

In another aspect, the present disclosure provides a host cell comprising any one of the polynucleotides described herein, or any one of the expression vectors described herein.

In some embodiments, the host cell is a prokaryotic cell. In some embodiments, the prokaryotic cell is an E. coli cell or a Bacillus cell. In some embodiments, the host cell is a eukaryotic cell. In some embodiments, the eukaryotic cell is selected from the group consisting of a yeast cell, an insect cell, and a mammalian cell. In some embodiments, the mammalian cell is selected from the group consisting of a CHO cell, a HeLa cell, and a 293 cell.

In another aspect, the present disclosure provides a population of cells comprising two or more of any of the host cells described herein.

In another aspect, the present disclosure provides a method of producing any one of the fusion proteins described herein, or any one of the polypeptides described herein, comprising culturing any one of the host cells described herein, under conditions such that the fusion protein is produced.

In another aspect, the present disclosure provides a pharmaceutical composition comprising an effective amount of any one of the fusion proteins described herein, any one of the polypeptides described herein, any one of the polynucleotides described herein, or any one of the expression vectors described herein.

In some embodiments, the fusion protein has a circulating half-life of at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, or at least 23 days. In some embodiments, the fusion protein has a circulating half-life of at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, or at least 23 days when administered (e.g., to a human). In some embodiments, the fusion protein has bioavailability of at least 40%, at least 45%, at least 50%, at least 55%, at least 60%, at least 65%, or at least 70% when administered (e.g., to a human). In some embodiments, administration of the pharmaceutical composition is via intravenous administration or subcutaneous administration.

In another aspect, the present disclosure provides a method of enhancing a relaxin-2-related activity in a primary cell, comprising contacting the primary cell with any one of the fusion proteins described herein, thereby enhancing relaxin-2-related activity in the cell.

In some embodiments, the fusion protein activates relaxin-2 receptor (RXFP1) on a cell surface.

In some embodiments, the method elevates cAMP levels in the primary cell, inducing vasodilation, inducing the expression of angiogenic factors, inducing the expression of MMPs, and inducing collagen degradation.

In some embodiments, the primary cell is selected from the group consisting of endothelial cells, vascular smooth muscle cells, other vascular cells, cardiomyocytes, other cardiac cells, and fibroblasts.

In some embodiments, the primary cell is within a subject. In some embodiments, the subject has a relaxin-2-associated disorder. In some embodiments, the relaxin-2-associated disorder is selected from the group consisting of kidney diseases, fibrotic diseases, and cardiovascular diseases. In some embodiments, the disorder is selected from the group consisting of pulmonary hypertension, pulmonary arterial hypertension (PAH), pulmonary hypertension due to left heart disease (PH-LHD), combined precapillary and postcapillary pulmonary hypertension (CpcPH), isolated postcapillary pulmonary hypertension (IpcPH), heart failure, heart failure with preserved ejection fraction (HFpEF), heart failure with mid-range ejection fraction (HFmrEF), heart failure with reduced ejection fraction (HFrEF), valvular heart disease, joint disease, frozen shoulder (also known as adhesive capsulitis), kidney disease, chronic kidney disease, and hypertensive kidney disease.

In some embodiments, the disorder is combined precapillary and postcapillary pulmonary hypertension (CpcPH) with heart failure with preserved ejection fraction (HFpEF). In some embodiments, the disorder is isolated postcapillary pulmonary hypertension (IpcPH) with heart failure with preserved ejection fraction (HFpEF). In some embodiments, the disorder is combined precapillary and postcapillary pulmonary hypertension (CpcPH) with heart failure with mid-range ejection fraction (HFmrEF). In some embodiments, the disorder is isolated postcapillary pulmonary hypertension (IpcPH) with heart failure with mid-range ejection fraction (HFmrEF).

In another aspect, the present disclosure provides a method of treating a relaxin-associated disorder in a subject in need thereof, comprising administering to the subject an effective amount of any one of the fusion proteins described herein, any one of the polynucleotides described herein, any one of the expression vectors described herein, or any one of the pharmaceutical compositions described herein, thereby treating the relaxin-associated disorder.

In some embodiments, the relaxin-2-associated disorder is selected from the group consisting of kidney diseases, fibrotic diseases, and cardiovascular diseases. In some embodiments, the disorder is selected from the group consisting of pulmonary hypertension, pulmonary arterial hypertension (PAH), pulmonary hypertension due to left heart disease (PH-LHD), combined precapillary and postcapillary pulmonary hypertension (CpcPH), isolated postcapillary pulmonary hypertension (IpcPH), heart failure, heart failure with preserved ejection fraction (HFpEF), heart failure with mid-range ejection fraction (HFmrEF), heart failure with reduced ejection fraction (HFrEF), kidney disease, chronic kidney disease, and hypertensive kidney disease. In some embodiments, the method decreases arterial pressure, increases renal artery blood flow, increases cardiac filling at diastole, resolves established fibrosis, and/or suppresses new fibrosis development in the subject.

In some embodiments, the method increases renal plasma flow in the subject. In some embodiments, the increase in the renal plasma flow in the subject persists after 1 week, 2 weeks, 3 weeks, 4 weeks, 5 weeks, or 1 month after a single administration of the fusion protein. In some embodiments, the increase in the renal plasma flow in the subject is maintained by at least about 30%, at least about 40%, at least about 50%, at least about 60%, at least about 70%, at least about 75%, at least about 80%, at least about 85%, at least about 90%, or at least about 95% 1 week, 2 weeks, 3 weeks, 4 weeks, 5 weeks, or 1 month after a single administration of the fusion protein.

In some embodiments, the disorder is combined precapillary and postcapillary pulmonary hypertension (CpcPH) with heart failure with preserved ejection fraction (HFpEF). In some embodiments, the disorder is isolated postcapillary pulmonary hypertension (IpcPH) with heart failure with preserved ejection fraction (HFpEF). In some embodiments, the disorder is combined precapillary and postcapillary pulmonary hypertension (CpcPH) with heart failure with mid-range ejection fraction (HFmrEF). In some embodiments, the disorder is isolated postcapillary pulmonary hypertension (IpcPH) with heart failure with mid-range ejection fraction (HFmrEF).

In some embodiments, the subject is administered the fusion protein by intravenous administration. In some embodiments, the subject is administered from about 0.1 mg/kg to about 20 mg/kg of the fusion protein. In some embodiments, the subject is administered about 0.3 mg/kg of the fusion protein. In some embodiments, the subject is administered about 1 mg/kg of the fusion protein. In some embodiments, the subject is administered about 3 mg/kg of the fusion protein. In some embodiments, the subject is administered about 10 mg/kg of the fusion protein.

In some embodiments, the subject is administered the fusion protein by intravenous infusion. In some embodiments, the subject is administered the fusion protein by intravenous infusion over 30 minutes. In some embodiments, the subject is administered the fusion protein by intravenous infusion over 60 minutes. In some embodiments, the subject is administered the fusion protein by intravenous infusion over 30 to 60 minutes.

In some embodiments, the subject is administered the fusion protein by subcutaneous administration. In some embodiments, the subject is administered about 100 mg to about 1500 mg of the fusion protein. In some embodiments, the subject is administered about 150 mg of the fusion protein. In some embodiments, the subject is administered at least 150 mg of the fusion protein. In some embodiments, the subject is administered about 300 mg of the fusion protein. In some embodiments, the subject is administered about 600 mg of the fusion protein.

In some embodiments, the subject is administered the fusion protein once every 1 week, 2 weeks, 3 weeks, 4 weeks, 5 weeks, or 1 month.

### BRIEF DESCRIPTION OF THE DRAWINGS

FIGS. 1A-1C are graphs depicting cAMP response induced by SEQ ID NO: 87 and wild-type (WT) human relaxin-2 in HEK293 cells transiently expressing human (FIG. 1A), rat (FIG. 1B), and monkey (FIG. 1C) RXFP1, respectively.

FIGS. 2A-2C are graphs depicting the pharmacokinetic (PK) values obtained by measuring the concentration of the various relaxin-2 fusion protein analogs (using human Fc levels as a proxy) as indicated in the serum of rats following a 5 mg/kg intravenous (IV) injection of the respective protein analog over time.

FIG. 3 is a graph depicting the change in renal arterial blood flow (RABF) compared to baseline, over time, in rats administered the various relaxin-2 fusion protein analogs as indicated.

FIGS. 4A and 4B are graphs depicting the changes to RABF (FIG. 4A) and serum levels of fusion protein (FIG. 4B; using human Fc levels as a proxy) in response to dose of SEQ ID NO: 87 or SEQ ID NO: 497 as indicated, over time. FIG. 4C is a graph depicting serum PK as a function of increase in RABF (baseline subtracted).

FIGS. 5A and 5B are graphs depicting that a low dose of SEQ ID NO: 87 increases and maintains RABF in treated rats significantly more than SEQ ID NO: 497. FIG. 5A shows the increase in RABF in rats treated with SEQ ID NO: 87 or SEQ ID NO: 497 over time. FIG. 5B shows that rats treated with SEQ ID NO: 87 demonstrate significant increase in RABF compared to SEQ ID NO: 497 by area under the curve analysis.

FIGS. 6A and 6B are graphs depicting the effect of SEQ ID NO: 87 on right ventricular systolic pressure (RVSP) following 10 mg/kg intravenous treatment of SEQ ID NO: 87 for three weeks in MCT-induced rats (MCT), with (FIG. 6A) or without (FIG. 6B) B cell depletion using an anti-CD20 antibody (no CD20 or +CD20). Sildenafil was used as a positive control in the non-B cell depleted animals.

FIGS. 7A and 7B are graphs depicting the effect of SEQ ID NO: 87 on mean pulmonary arterial pressure (mPAP) following 10 mg/kg intravenous treatment of SEQ ID NO: 87 for three weeks in MCT-induced rats (MCT), with (FIG. 7A) or without (FIG. 7B) B cell depletion using an anti-CD20 antibody (no CD20 or +CD20). Sildenafil was used as a positive control in the non-B cell depleted animals.

FIGS. 8A and 8B are graphs depicting the effect of SEQ ID NO: 87 on the Fulton Index following 10 mg/kg intravenous treatment of SEQ ID NO: 87 for three weeks in MCT-induced rats (MCT), with (FIG. 8A) or without (FIG. 8B) B cell depletion using an anti-CD20 antibody (no CD20 or +CD20). Sildenafil was used as a positive control in the non-B cell depleted animals.

FIGS. 9A and 9B are graphs depicting the effect of SEQ ID NO: 87 on serum NT-pro-BNP levels following 10 mg/kg intravenous treatment of SEQ ID NO: 87 for three weeks in MCT-induced rats (MCT), with (FIG. 9A) or without (FIG. 9B) B cell depletion using an anti-CD20 antibody (no CD20 or +CD20). Sildenafil was used as a positive control in the non-B cell depleted animals.

FIGS. 10A and 10B are graphs depicting the results of histopathological analysis of the effect of SEQ ID NO: 87 on lung inflammation (FIG. 10A) and pulmonary arterial muscularization (FIG. 10B) following 10 mg/kg intravenous treatment of SEQ ID NO: 87 for three weeks in MCT-induced rats (MCT), with B cell depletion using an anti-CD20 antibody (+anti-CD20). *: p<0.05; **: p<0.01; ****: p<0.0001 using a nonparametric 1-way analysis of variance with post hoc Dunn's multiple comparisons tests.

FIG. 11 is a graph depicting the effect of SEQ ID NO: 87 on mortality following 10 mg/kg intravenous treatment of SEQ ID NO: 87 for three weeks in MCT-induced rats (MCT), with or without B cell depletion using an anti-CD20 antibody (no CD20 or +CD20). Sildenafil was used as a positive control in the non-B cell depleted animals.

FIG. 12 is a graph depicting the effect of SEQ ID NO: 496 and SEQ ID NO: 313 on collagen deposition in renal parenchyma in a mouse unilateral ureteral obstruction (UUO) model, according to aspects of the present disclosure. Mice underwent UUO surgery and were treated with vehicle (PBS; n=10), 20 mg/kg SEQ ID NO: 496 (n=10), 10 mg/kg SEQ ID NO: 313 (n=10), or 20 mg/kg SEQ ID NO: 313 (n=10). Also shown are control mice that underwent a sham surgery and were treated with vehicle (PBS; n=5). Following treatment, obstructed kidneys were harvested and fixed for histology. Collagen was detected via immunolabeling. Depicted is a quantification of collagen levels as a percentage of total immunolabeled area. *: p<0.05; ****: p<0.0001.

FIG. 13 is a graph depicting the effect of SEQ ID NO: 87 on collagen deposition in kidney cortex in a mouse UUO model, according to aspects of the present disclosure. Mice underwent UUO surgery and were treated with vehicle (PBS; n=8) or 10 mg/kg SEQ ID NO: 87 (n=8). Also shown are control mice that underwent a sham surgery and were treated with vehicle (PBS; n=8). Following treatment, obstructed kidneys were harvested and fixed for histology. Collagen was detected via immunolabeling. Depicted is a quantification of collagen levels as a percentage of total immunolabeled area. ****: p<0.0001; *: p=0.02.

FIG. 14 is a graph depicting the effect of SEQ ID NO: 87 on TNFα levels in kidney cortex in a mouse UUO model, according to aspects of the present disclosure. Mice were treated as described for FIG. 13, and TNFα levels were quantified in protein lysates via electrochemiluminescence assay. ****: p<0.0001; ***: p<0.001.

FIG. 15 is a graph depicting the effect of SEQ ID NO: 87 on isoproterenol-induced cardiac hypertrophy, according to aspects of the present disclosure. Mice were treated with vehicle (n=10), isoproterenol (n=10), or isoproterenol and SEQ ID NO: 87 (n=6). Following treatment, body weight and heart rate was measured for each mouse. Depicted is heart weight normalized by body weight (HW/BW) for each group. ****: p<0.0001.

FIG. 16 is a graph depicting the effect of SEQ ID NO: 87 on isoproterenol-induced fibrosis, according to aspects of the present disclosure. Mice were treated as described for FIG. 15, and collagen content was quantified using a hydroxyproline assay. ****: p<0.0001; ***: p<0.001.

FIGS. 17A and 17B are graphs depicting PK (FIG. 17A) and PD (FIG. 17B) data of healthy human patients administered a single 0.3 mg/kg IV dose of SEQ ID NO: 87. FIG. 17A shows the concentration of SEQ ID NO: 87 over time in dosed patients (solid lines), as well as the PK profile of SEQ ID NO: 87 as predicted using non-human primate modeling (dashed line). FIG. 17B shows the change in renal plasma flow over baseline on days 2, 8, and 17, in healthy patients dosed with SEQ ID NO: 87 or placebo (PBO).

FIG. 18 is a graph depicting PK data of healthy human patients administered a single 150 mg SC dose of SEQ ID NO: 87, showing the concentration of SEQ ID NO: 87 over time in dosed patients.

### DETAILED DESCRIPTION

The therapeutic potential of relaxin-2 was highlighted in the RELAX-AHF trials (see, e.g., Teerlink et al., (2013) Lancet 381 (9860): 29-39). However, the therapeutic protein used, human relaxin-2 (Serelaxin), had not been modified in any way to extend half-life in vivo, and the protein had to be administered by continuous IV infusion over a 48-hour period. Half-life extended versions of relaxin-2 have been generated via fusion of the peptide hormone to human IgG1 Fc or to an albumin binding nanobody, but such fusion proteins have shown extremely rapid clearance from plasma. The present disclosure is based in part on the discovery by the inventors that reducing the positive charge and heparin binding of relaxin-2 results in greatly improved pharmacokinetic and pharmacodynamic profiles.

The disclosure provides fusion proteins comprising a human relaxin-2 B chain, or a derivative thereof, and a human relaxin-2 A chain, or a derivative thereof, joined by a peptide linker, wherein the fusion proteins have high in vivo circulating half-life when administered to mammals. In some embodiments, the in vivo circulating half-life of the fusion proteins provided in this disclosure is greater than 2 hours. In some embodiments, the fusion proteins provided in this disclosure have low pI. In some embodiments, the pI of the fusion proteins provided in this disclosure is less than 8.5. In some embodiments, the low pI of the fusion proteins provided in this disclosure is caused by acidic amino acid residues present in the peptide linker. In some embodiments, the peptide linker of the fusion protein comprises 2 or more acidic amino acids. In some embodiments, the peptide linker is 10-15 total amino acids in length.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting. The section headings used herein are for organizational purposes only and are not to be construed as limiting the subject matter described.

The term “polynucleotide” as used herein refers to a polymer of DNA or RNA. The polynucleotide sequence can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified polynucleotide sequence. Polynucleotide sequences include, but are not limited to, all polynucleotide sequences which are obtained by any means available in the art, including, without limitation, recombinant means, e.g., the cloning of polynucleotide sequences from a recombinant library or a cell genome, using ordinary cloning technology and polymerase chain reaction, and the like, and by synthetic means.

The terms “protein” and “polypeptide” are used interchangeably herein and refer to a polymer of amino acids connected by one or more peptide bonds. As used herein, “amino acid sequence” refers to the information describing the relative order and identity of amino acid residues which make up a polypeptide.

As used herein, the term “an amino acid sequence that has 0, 1, 2, 3, 4, or 5 amino acid modifications” with reference to an amino acid sequence, refers to an amino acid sequence that comprises up to 5 amino acid substitutions, alterations, inversions, additions, or deletions compared to a reference amino acid sequence.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F, (1990) PNAS 87:2264-2268, modified as in Karlin S & Altschul S F, (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., at score=100, wordlength=12 to obtain nucleotide sequences homologous to a nucleic acid molecule described herein. BLAST protein searches can be performed with the XBLAST program parameters set, e.g., at score=50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules. Id. When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, (1988) CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “linked to” refers to covalent or noncovalent binding between two molecules or moieties. The skilled worker will appreciate that when a first molecule or moiety is linked to a second molecule or moiety, the linkage need not be direct, but instead, can be via an intervening molecule or moiety.

As used herein, the terms “human relaxin-2 B chain” or “relaxin B chain” or “relaxin B” or “rel B” refer to a peptide comprising or consisting of the amino acid sequence as set forth in DSWMEEVIKLCGRELVRAQIAICGMSTWS (SEQ ID NO: 249) or derivatives thereof. In some embodiments, a derivative of a relaxin B chain comprises the amino acid sequence of SEQ ID NO: 154 with 1, 2, 3, 4, or 5 amino acid changes.

As used herein, the terms “human relaxin-2 A chain” or “relaxin A chain” or “relaxin A” or “rel A” refer to a peptide comprising or consisting of the amino acid sequence as set forth in QLYSALANKCCHVGCTKRSLARFC (SEQ ID NO: 257) or derivatives thereof. In some embodiments, a derivative of a relaxin A chain comprises the amino acid sequence of SEQ ID NO: 155 with 1, 2, 3, 4, or 5 amino acid changes.

As used herein, the term “linker peptide” refers to a peptide that links the relaxin A chain and the relaxin B chain in the fusion proteins described herein.

As used herein, the term “acidic amino acid” refers to an amino acid that has a carboxylic acid in its side chain. In some embodiments, the acidic amino acid is aspartate, glutamate, 2-aminoadipic acid, 2-aminobutyric acid or 2-aminopimelic acid. In some embodiments, acid amino acids include aspartate and glutamate.

As used herein, the term “non-acidic amino acid” refers to amino acids that are not acidic amino acids. In some embodiments, non-acidic amino acids include glycine, proline, and serine. In some embodiments, non-specific amino acids also include arginine, histidine, lysine, threonine, asparagine, glutamine, cysteine, alanine, valine, isoleucine, leucine, methionine, phenylalanine, tyrosine, and tryptophan.

As used herein, the term “IgG Fc” refers to the immunoglobulin G (IgG) fragment crystallizable (Fc) region. In some embodiments, the IgG Fc is the human IgG1, IgG2, IgG3, or IgG4 Fc region. In some embodiments, the IgG Fc is the IgG1 Fc region.

As used herein, the term “EU numbering system” refers to the EU numbering convention for the constant regions of an antibody, as described in Edelman, G. M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety.

As used herein, the term “relaxin-2 receptor,” “human relaxin-2 receptor,” “human relaxin receptor 1,” “RXFP1,” or “LGR7” is the native receptor of relaxin-2 in humans. In some embodiments, RXFP1 comprises the amino acid sequence shown in NCBI Reference Sequence: NP_067647.2, NP_001240656.1, NP_001240657.1, NP_001240658.1, NP_001240659.1, NP_001240661.1, NP_001240662.1, or NP_001350705.1 incorporated herein by reference in its entirety.

As used herein, the terms “treat,” “treating,” and “treatment” refer to therapeutic or preventative measures described herein. In some embodiments, the methods of “treatment” employ administration of a fusion protein to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In one embodiment, the subject is a human or non-human mammal. In one embodiment, the subject is a human.

As used herein, the term “pI” means the isoelectric point, i.e., the pH of a solution at which the next charge on a fusion protein is zero. In some embodiments, the pI is the calculated or theoretical pI. In some embodiments, the pI is measured experimentally by an instrument.

### Fusion Proteins

The disclosure provides fusion proteins comprising a human relaxin-2 B chain, or a derivative thereof, and a human relaxin-2 A chain, or a derivative thereof, linked by a peptide linker, wherein the fusion proteins have high in vivo circulating half-life when administered to mammals. In some embodiments, the fusion protein comprises, from N-terminus to C-terminus, a human relaxin-2 B chain, or a derivative thereof, a peptide linker and a human relaxin-2 A chain, or a derivative thereof. In some embodiments, the fusion protein comprises, from N-terminus to C-terminus, a human relaxin-2 A chain, or a derivative thereof, a peptide linker and a human relaxin-2 B chain, or a derivative thereof. In some embodiments, the fusion protein further comprises an IgG Fc. The IgG Fc is linked to the N-terminus or C-terminus of the human relaxin B chain-linker protein-human relaxin A chain fusion protein or the human relaxin A chain-linker protein-human relaxin B chain fusion protein. In some embodiments, the fusion proteins form homodimers via interaction between IgG Fc moieties. In some embodiments, the IgG Fc described above is replaced with PEG.

### Human Relaxin-2 B Chain Derivatives

The disclosure provides human relaxin-2 B chain derivatives, wherein the derivatives have 1, 2, 3, 4, or 5 amino acid changes when compared to the amino acid sequence of SEQ ID NO: 249. In some embodiments, the amino acid that corresponds with position 13 of SEQ ID NO: 249 must be arginine. In some embodiments, the amino acid that corresponds with position 17 of SEQ ID NO: 249 must be arginine. In some embodiments, the amino acid that corresponds with position 20 of SEQ ID NO: 249 must be isoleucine. In some embodiments, the amino acid that corresponds with position 13 of SEQ ID NO: 249 must be arginine; the amino acid that corresponds with position 17 of SEQ ID NO: 249 must be arginine; and the amino acid that corresponds with position 20 of SEQ ID NO: 249 must be isoleucine.

In some embodiments, the human relaxin-2 B chain derivatives comprise or consist of the following formula: DSWX₁₉EEVIKLCGRELVRAQIAICGX₂₀ST (SEQ ID NO: 250), wherein X₁₉ and X₂₀ are absent or any amino acid. In some embodiments, X₁₉ is methionine (M), glutamine (Q), glutamic acid (E), asparagine (N), aspartic acid (D), serine(S), or threonine (T). In some embodiments, X₁₉ is methionine (M), lysine (K) or glutamine (Q). In some embodiments, X₂₀ is methionine (M), lysine (K), glutamine (Q), or asparagine (N). In some embodiments, X₂₀ is methionine (M) or lysine (K). In some embodiments, X₂₀ is lysine (K). In some embodiments, X₁₉ is methionine (M), lysine (K) or glutamine (Q), and X₂₀ is methionine (M) or lysine (K).

The disclosure provides human relaxin-2 B chain derivatives, wherein the derivatives comprise an amino acid sequence that has 0, 1, 2, 3, 4, or 5 amino acid modifications relative to the amino acid sequence of SEQ ID NO: 1, wherein the amino acid at position 4 is not methionine (M), or the amino acid at position 25 of is not methionine (M). In some embodiments, the derivatives comprise an amino acid sequence that has 0, 1, 2, 3, 4, or 5 amino acid modifications relative to the amino acid sequence of SEQ ID NO: 1, wherein the amino acid at position 4 is not methionine (M), and the amino acid at position 25 of is not methionine (M). In some embodiments, the derivatives comprise an amino acid sequence that has 0, 1, 2, 3, 4, or 5 amino acid modifications relative to the amino acid sequence of SEQ ID NO: 1, wherein the amino acid at at least one of positions 4 or 25 of the first peptide is not methionine (M).

In some embodiments, the human relaxin-2 B chain derivatives comprise or consist of the following formula: DSX₁QEEVIX₂LCGRELVRAQIAICGX₃ST (SEQ ID NO: 7), wherein X₁ is tryptophan (W), tyrosine (Y), phenylalanine (F), leucine (L), isoleucine (I), valine (V), or alanine (A); X₂ is lysine (K), glutamine (Q), aspartic acid (D), glutamic acid (E), leucine (L), isoleucine (I), or tyrosine (Y); and X₃ is lysine (K) or glutamine (Q). In some embodiments, the human relaxin-2 B chain derivatives comprise or consist of the following formula: DSX₁QEEVIX₂LCGRELVRAQIAICGX₃ST (SEQ ID NO: 7), wherein X₁ is tryptophan (W), tyrosine (Y), phenylalanine (F), leucine (L), isoleucine (I), valine (V), or alanine (A); X₂ is lysine (K), glutamine (Q), aspartic acid (D), glutamic acid (E), leucine (L), isoleucine (I), or tyrosine (Y); and X₃ is methionine (M), lysine (K), glutamine (Q), or asparagine (N). In some embodiments, the human relaxin-2 B chain derivatives comprise or consist of the following formula: DSX₁QEEVIX₂LCGRELVRAQIAICGX₃ST (SEQ ID NO: 7), wherein X₁ is any amino acid except for methionine (M), histidine (H), and cysteine (C); X₂ is lysine (K), glutamine (Q), aspartic acid (D), glutamic acid (E), leucine (L), isoleucine (I), or tyrosine (Y); and X₃ is lysine (K) or glutamine (Q). In some embodiments, the human relaxin-2 B chain derivatives comprise or consist of the following formula: DSX₁QEEVIX₂LCGRELVRAQIAICGX₃ST (SEQ ID NO: 7), wherein X₁ is any amino acid except for methionine (M), histidine (H), and cysteine (C); X₂ is lysine (K), glutamine (Q), aspartic acid (D), glutamic acid (E), leucine (L), isoleucine (I), or tyrosine (Y); and X₃ is methionine (M), lysine (K), glutamine (Q), or asparagine (N).

In some embodiments, the human relaxin-2 B chain derivatives comprise an amino acid sequence that has 0, 1, 2, 3, 4, or 5 amino acid modifications relative to the amino acid sequence of SEQ ID NO: 502. In some embodiments, the human relaxin-2 B chain derivatives comprise or consist of the following formula: X₁₁LCGRELVRAQIAIC (SEQ ID NO: 505), wherein X₁₁ is lysine (K), glutamine (Q), aspartic acid (D), glutamic acid (E), leucine (L), isoleucine (I), or tyrosine (Y).

In some embodiments, the human relaxin-2 B chain derivatives used in the fusion proteins described herein do not include the amino acid sequences of SEQ ID NOs: 251-254 as set forth below:

(SEQ ID NO: 251)
DSWKEEVIKLCGRELVRAQIAICGKSTAS;
(SEQ ID NO: 252)
DSWKEEVIKLCGRELVRAQIAICGKSTWS;
(SEQ ID NO: 253)
DSWMEEVIKLCGRELVRAQIAICGKSTAS;
and
(SEQ ID NO: 254
DSWMEEVIKLCGRELVRAQIAICGKSTWS.

In some embodiments, the human relaxin-2 B chain derivatives are from 22, 23, 24, 25, 26, 27, 28, 29, 30, 31, or 32 amino acids in length. In some embodiments, the human relaxin-2 B chain derivatives are 25, 26, 27, 28, or 29 amino acids in length. In some embodiments, the human relaxin-2 B chain derivatives are 27 amino acids in length. In some embodiments, the human relaxin-2 B chain derivatives are 15-29 amino acids in length. In some embodiments, the human relaxin-2 B chain derivatives are 15, 16, 17, 18, 19, 20, 21, 22, 23, 24, 25, 26, 27, 28, or 29 amino acids in length.

In some embodiments, the human relaxin-2 B chain derivatives comprise or consist of the amino acid sequences shown in Table 1, below.

TABLE 1
Human Relaxin-2 B Chain Derivative Sequences

| SEQ ID NO: | Amino Acid Sequence |
|---|---|
| 1 | DSWQEEVIKLCGRELVRAQIAICGKST |
| 2 | DSWQEEVIKLCGRELVRAQIAICGQST |
| 3 | DSYQEEVIKLCGRELVRAQIAICGKST |
| 4 | DSFQEEVIKLCGRELVRAQIAICGKST |
| 5 | DSLQEEVIKLCGRELVRAQIAICGKST |
| 6 | DSIQEEVIKLCGRELVRAQIAICGKST |
| 249 | DSWMEEVIKLCGRELVRAQIAICGMSTWS |
| 255 | DSWKEEVIKLCGRELVRAQIAICGKST |
| 256 | DSWMEEVIKLCGRELVRAQIAICGKST |
| 502 | KLCGRELVRAQIAIC |

In some embodiments, the human relaxin-2 B chain derivatives comprise or consist of SEQ ID NO: 1. In some embodiments, the human relaxin-2 B chain derivatives comprise or consist of SEQ ID NO: 2. In some embodiments, the human relaxin-2 B chain derivatives comprise or consist of SEQ ID NO: 3. In some embodiments, the human relaxin-2 B chain derivatives comprise or consist of SEQ ID NO: 4. In some embodiments, the human relaxin-2 B chain derivatives comprise or consist of SEQ ID NO: 5. In some embodiments, the human relaxin-2 B chain derivatives comprise or consist of SEQ ID NO: 6. In some embodiments, the human relaxin-2 B chain derivatives comprise or consist of SEQ ID NO: 249. In some embodiments, the human relaxin-2 B chain derivatives comprise or consist of SEQ ID NO: 255. In some embodiments, the human relaxin-2 B chain derivatives comprise or consist of SEQ ID NO: 256. In some embodiments, the human relaxin-2 B chain derivatives comprise or consist of SEQ ID NO: 502.

In some embodiments, the human relaxin-2 B chain derivatives comprise or consist of SEQ ID NO: 1, wherein the amino acid at position 9 is lysine, (K), glutamine (Q), aspartic acid (D), glutamic acid (E), leucine (L), isoleucine (I), or tyrosine (Y). In some embodiments, the human relaxin-2 B chain derivatives comprise or consist of SEQ ID NO: 2, wherein the amino acid at position 9 is lysine, (K), glutamine (Q), aspartic acid (D), glutamic acid (E), leucine (L), isoleucine (I), or tyrosine (Y). In some embodiments, the human relaxin-2 B chain derivatives comprise or consist of SEQ ID NO: 3, wherein the amino acid at position 9 is lysine, (K), glutamine (Q), aspartic acid (D), glutamic acid (E), leucine (L), isoleucine (I), or tyrosine (Y). In some embodiments, the human relaxin-2 B chain derivatives comprise or consist of SEQ ID NO: 4, wherein the amino acid at position 9 is lysine, (K), glutamine (Q), aspartic acid (D), glutamic acid (E), leucine (L), isoleucine (I), or tyrosine (Y). In some embodiments, the human relaxin-2 B chain derivatives comprise or consist of SEQ ID NO: 5, wherein the amino acid at position 9 is lysine, (K), glutamine (Q), aspartic acid (D), glutamic acid (E), leucine (L), isoleucine (I), or tyrosine (Y). In some embodiments, the human relaxin-2 B chain derivatives comprise or consist of SEQ ID NO: 6, wherein the amino acid at position 9 is lysine, (K), glutamine (Q), aspartic acid (D), glutamic acid (E), leucine (L), isoleucine (I), or tyrosine (Y). In some embodiments, the human relaxin-2 B chain derivatives comprise or consist of SEQ ID NO: 249, wherein the amino acid at position 9 is lysine, (K), glutamine (Q), aspartic acid (D), glutamic acid (E), leucine (L), isoleucine (I), or tyrosine (Y). In some embodiments, the human relaxin-2 B chain derivatives comprise or consist of SEQ ID NO: 255, wherein the amino acid at position 9 is lysine, (K), glutamine (Q), aspartic acid (D), glutamic acid (E), leucine (L), isoleucine (I), or tyrosine (Y). In some embodiments, the human relaxin-2 B chain derivatives comprise or consist of SEQ ID NO: 256, wherein the amino acid at position 9 is lysine, (K), glutamine (Q), aspartic acid (D), glutamic acid (E), leucine (L), isoleucine (I), or tyrosine (Y). In some embodiments, the human relaxin-2 B chain derivatives comprise or consist of SEQ ID NO: 502, wherein the amino acid at position 1 is lysine, (K), glutamine (Q), aspartic acid (D), glutamic acid (E), leucine (L), isoleucine (I), or tyrosine (Y).

In some embodiments, the human relaxin-2 B chain derivatives further comprise two residues at the C-terminal end. For example, SEQ ID NOs: 1-7, 250, 255, and 256 can further comprise two residues at the C-terminal end, e.g., the tryptophan (W) and serine(S) at the C-terminal end of SEQ ID NO: 249. In some embodiments, the human relaxin-2 B chain derivatives further comprise an X amino acid and serine(S) at the C-terminal end, wherein the X amino acid can be any amino acid except cysteine (C). Accordingly, in some embodiments, the C-terminal end of the human relaxin-2 B chain derivatives are XS, wherein X is any amino acid except cysteine (C).

In some embodiments, the human relaxin-2 B chain derivatives further comprise one or more substitutions that improve the stability of the relaxin-2 B chain derivatives, e.g., stability of the relaxin-2 B chain derivatives after light or heat exposure, as measured by methods known in the art, e.g., SEC to assess aggregate formation, and CE-SDS to assess purity. In some embodiments, the amino acid in the human relaxin-2 B chain derivatives corresponding to amino acid position 3 in SEQ ID NO: 3 is a tyrosine (Y).

### Human Relaxin-2 A Chain Derivatives

The disclosure provides human relaxin-2 A chain derivatives, wherein the derivatives have 1, 2, 3, 4, or 5 amino acid changes when compared to the amino acid sequence of SEQ ID NO: 257. In some embodiments, the amino acid that corresponds with position 3 of SEQ ID NO: 257 must be tyrosine. In some embodiments, the amino acid that corresponds with position 23 of SEQ ID NO: 257 must be phenylalanine. In some embodiments, the amino acid that corresponds with position 3 of SEQ ID NO: 257 must be tyrosine; and the amino acid that corresponds with position 23 of SEQ ID NO: 257 must be phenylalanine.

In some embodiments, the human relaxin-2 A chain derivatives comprise or consist of the following formula: X₂₁QX₂₂YSALANKCCHVGCTKRSLAX₂₃FC (SEQ ID NO: 258), wherein X₂₁, X₂₂, and X₂₃ are absent or any amino acid. In some embodiments, X₂₁ is arginine (R), lysine (K), glutamine (Q), asparagine (N), histidine (H), serine(S), threonine (T), proline (P), glycine (G), or absent. In some embodiments, X₂₁ is arginine (R), glycine (G), or absent. In some embodiments, X₂₁ is arginine (R) or absent. In some embodiments, X₂₂ is leucine (L), aspartic acid (D), glutamic acid (E), asparagine (N), glutamine (Q), serine(S), or threonine (T). In some embodiments, X₂₂ is leucine (L) or aspartic acid (D). In some embodiments, X₂₃ is arginine (R), glutamine (Q), glutamic acid (E), aspartic acid (D), asparagine (N), serine(S), or threonine (T). In some embodiments, X₂₃ is arginine (R), glutamine (Q), or glutamic acid (E). In some embodiments, X₂₁ is arginine (R) or absent, X₂₂ is leucine (L) or aspartic acid (D), and X₂₃ is arginine (R), glutamine (Q), or glutamic acid (E).

The disclosure provides human relaxin-2 A chain derivatives, wherein the derivatives comprise an amino acid sequence that has 0, 1, 2, 3, 4, or 5 amino acid modifications relative to the amino acid sequence of SEQ ID NO: 8 wherein the amino acid at position 22 of the second peptide is not arginine (R).

In some embodiments, the human relaxin-2 A chain derivatives comprise or consist of the following formula: QLYSALANX₄CCX₅VGCTX₆X₇SLAQFC (SEQ ID NO: 16), wherein X₄ is lysine (K), glutamine (Q), aspartic acid (D), glutamic acid (E), leucine (L), isoleucine (I), or tyrosine (Y); X₅ is histidine (H), lysine (K), glutamine (Q), tyrosine (Y), leucine (L), asparagine (N), isoleucine (I), serine(S), threonine (T), or phenylalanine (F); X₆ is lysine (K), glutamine (Q), aspartic acid (D), glutamic acid (E), leucine (L), isoleucine (I), or tyrosine (Y); and X₇ is glutamine (Q), aspartic acid (D), glutamic acid (E), leucine (L), isoleucine (I), tyrosine (Y), or arginine (R). In some embodiments, the human relaxin-2 A chain derivatives comprise or consist of the following formula: QLYSALANX₄CCX₅VGCTX₆X₇SLAQFC (SEQ ID NO: 16), wherein X₄ is lysine (K), glutamine (Q), aspartic acid (D), glutamic acid (E), leucine (L), isoleucine (I), or tyrosine (Y); X₅ is any amino acid except methionine (M), tryptophan (W), and cysteine (C); X₆ is lysine (K), glutamine (Q), aspartic acid (D), glutamic acid (E), leucine (L), isoleucine (I), or tyrosine (Y); and X₇ is glutamine (Q), aspartic acid (D), glutamic acid (E), leucine (L), isoleucine (I), tyrosine (Y), or arginine (R).

In some embodiments, the human relaxin-2 A chain derivatives comprise an amino acid sequence that has 0, 1, 2, 3, 4, or 5 amino acid modifications relative to the amino acid sequence of SEQ ID NO: 503 or 504. In some embodiments, the human relaxin-2 A chain derivatives comprise or consist of the following formula: X₁₂CCX₁₃VGCTX₁₄X₁₅SLAX₁₆FC (SEQ ID NO: 506), wherein X₁₂ is lysine (K), glutamine (Q), aspartic acid (D), glutamic acid (E), leucine (L), isoleucine (I), or tyrosine (Y); X₁₃ is histidine (H), lysine (K), glutamine (Q), tyrosine (Y), leucine (L), asparagine (N), isoleucine (I), serine(S), threonine (T), or phenylalanine (F); X₁₄ is lysine (K), glutamine (Q), aspartic acid (D), glutamic acid (E), leucine (L), isoleucine (I), or tyrosine (Y); X₁₅ is glutamine (Q), aspartic acid (D), glutamic acid (E), leucine (L), isoleucine (I), tyrosine (Y), or arginine (R); and X₁₆ is arginine (R) or glutamine (Q). In some embodiments, the human relaxin-2 A chain derivatives comprise or consist of the following formula: X₁₂CCX₁₃VGCTX₁₄X₁₅SLAX₁₆FC (SEQ ID NO: 506), wherein X₁₂ is lysine (K), glutamine (Q), aspartic acid (D), glutamic acid (E), leucine (L), isoleucine (I), or tyrosine (Y); X₁₃ is histidine (H), lysine (K), glutamine (Q), tyrosine (Y), leucine (L), asparagine (N), isoleucine (I), serine(S), threonine (T), or phenylalanine (F); X₁₄ is lysine (K), glutamine (Q), aspartic acid (D), glutamic acid (E), leucine (L), isoleucine (I), or tyrosine (Y); X₁₅ is glutamine (Q), aspartic acid (D), glutamic acid (E), leucine (L), isoleucine (I), tyrosine (Y), or arginine (R); and X₁₆ is arginine (R), glutamine (Q), glutamic acid (E), aspartic acid (D), asparagine (N), serine(S), or threonine (T). In some embodiments, the human relaxin-2 A chain derivatives comprise or consist of the following formula: X₁₂CCX₁₃VGCTX₁₄X₁₅SLAX₁₆FC (SEQ ID NO: 506), wherein X₁₂ is lysine (K), glutamine (Q), aspartic acid (D), glutamic acid (E), leucine (L), isoleucine (I), or tyrosine (Y); X₁₃ is any amino acid except methionine (M), tryptophan (W), and cysteine (C); X₁₄ is lysine (K), glutamine (Q), aspartic acid (D), glutamic acid (E), leucine (L), isoleucine (I), or tyrosine (Y); X₁₅ is glutamine (Q), aspartic acid (D), glutamic acid (E), leucine (L), isoleucine (I), tyrosine (Y), or arginine (R); and X₁₆ is arginine (R) or glutamine (Q). In some embodiments, the human relaxin-2 A chain derivatives comprise or consist of the following formula: X₁₂CCX₁₃VGCTX₁₄X₁₅SLAX₁₆FC (SEQ ID NO: 506), wherein X₁₂ is lysine (K), glutamine (Q), aspartic acid (D), glutamic acid (E), leucine (L), isoleucine (I), or tyrosine (Y); X₁₃ is any amino acid except methionine (M), tryptophan (W), and cysteine (C); X₁₄ is lysine (K), glutamine (Q), aspartic acid (D), glutamic acid (E), leucine (L), isoleucine (I), or tyrosine (Y); X₁₅ is glutamine (Q), aspartic acid (D), glutamic acid (E), leucine (L), isoleucine (I), tyrosine (Y), or arginine (R); and X₁₆ is arginine (R), glutamine (Q), glutamic acid (E), aspartic acid (D), asparagine (N), serine(S), or threonine (T).

In some embodiments, the human relaxin-2 A chain derivatives are from 19, 20, 21, 22, 23, 24, 25, 26, 27, 28, 29, or 30 amino acids in length. In some embodiments, the human relaxin-2 A chain derivatives are 22, 23, 24, 25, or 26 amino acids in length. In some embodiments, the human relaxin-2 A chain derivatives are 24 amino acids in length. In some embodiments, the human relaxin-2 A chain derivatives are 25 amino acids in length. In some embodiments, the human relaxin-2 A chain derivatives are 16-25 amino acids in length. In some embodiments, the human relaxin-2 A chain derivatives are 16, 17, 18, 19, 20, 21, 22, 23, 24, or 25 amino acids in length.

In some embodiments, the human relaxin-2 A chain derivatives comprise or consist of the amino acid sequences shown in Table 2, below.

TABLE 2
Human Relaxin-2 A Chain Derivative Sequences

| SEQ ID NO: | Amino Acid Sequence |
|---|---|
| 8 | QLYSALANKCCHVGCTKRSLAQFC |
| 9 | QLYSALANKCCHVGCTKQSLAQFC |
| 10 | QLYSALANKCCYVGCTKRSLAQFC |
| 11 | QLYSALANKCCLVGCTKRSLAQFC |
| 12 | QLYSALANKCCQVGCTKRSLAQFC |
| 13 | QLYSALANKCCKVGCTKRSLAQFC |
| 14 | QLYSALANKCCYVGCTKQSLAQFC |
| 15 | QLYSALANKCCKVGCTKQSLAQFC |
| 257 | QLYSALANKCCHVGCTKRSLARFC |
| 259 | RQLYSALANKCCHVGCTKRSLARFC |
| 260 | RQLYSALANKCCHVGCTKRSLAQFC |
| 261 | RQLYSALANKCCHVGCTKRSLAEFC |
| 503 | KCCHVGCTKRSLARFC |
| 504 | KCCHVGCTKRSLAQFC |
| 507 | QLYSALANKCCQVGCTKQSLAQFC |
| 550 | QLYSALANKCCHVGCTKRSLAEFC |
| 551 | RQLYSALANKCCHVGCTKQSLAQFC |
| 552 | RQLYSALANKCCYVGCTKRSLAQFC |
| 553 | RQLYSALANKCCLVGCTKRSLAQFC |
| 554 | RQLYSALANKCCQVGCTKRSLAQFC |
| 555 | RQLYSALANKCCKVGCTKRSLAQFC |
| 556 | RQLYSALANKCCYVGCTKQSLAQFC |
| 557 | RQLYSALANKCCKVGCTKQSLAQFC |
| 558 | RQLYSALANKCCQVGCTKQSLAQFC |

In some embodiments, the human relaxin-2 A chain derivatives comprise or consist of SEQ ID NO: 8. In some embodiments, the human relaxin-2 A chain derivatives comprise or consist of SEQ ID NO: 9. In some embodiments, the human relaxin-2 A chain derivatives comprise or consist of SEQ ID NO: 10. In some embodiments, the human relaxin-2 A chain derivatives comprise or consist of SEQ ID NO: 11. In some embodiments, the human relaxin-2 A chain derivatives comprise or consist of SEQ ID NO: 12. In some embodiments, the human relaxin-2 A chain derivatives comprise or consist of SEQ ID NO: 13. In some embodiments, the human relaxin-2 A chain derivatives comprise or consist of SEQ ID NO: 14. In some embodiments, the human relaxin-2 A chain derivatives comprise or consist of SEQ ID NO: 15. In some embodiments, the human relaxin-2 A chain derivatives comprise or consist of SEQ ID NO: 257. In some embodiments, the human relaxin-2 A chain derivatives comprise or consist of SEQ ID NO: 259. In some embodiments, the human relaxin-2 A chain derivatives comprise or consist of SEQ ID NO: 260. In some embodiments, the human relaxin-2 A chain derivatives comprise or consist of SEQ ID NO: 261. In some embodiments, the human relaxin-2 A chain derivatives comprise or consist of SEQ ID NO: 503. In some embodiments, the human relaxin-2 A chain derivatives comprise or consist of SEQ ID NO: 504. In some embodiments, the human relaxin-2 A chain derivatives comprise or consist of SEQ ID NO: 507. In some embodiments, the human relaxin-2 A chain derivatives comprise or consist of SEQ ID NO: 550. In some embodiments, the human relaxin-2 A chain derivatives comprise or consist of SEQ ID NO: 551. In some embodiments, the human relaxin-2 A chain derivatives comprise or consist of SEQ ID NO: 552. In some embodiments, the human relaxin-2 A chain derivatives comprise or consist of SEQ ID NO: 553. In some embodiments, the human relaxin-2 A chain derivatives comprise or consist of SEQ ID NO: 554. In some embodiments, the human relaxin-2 A chain derivatives comprise or consist of SEQ ID NO: 555. In some embodiments, the human relaxin-2 A chain derivatives comprise or consist of SEQ ID NO: 556. In some embodiments, the human relaxin-2 A chain derivatives comprise or consist of SEQ ID NO: 557. In some embodiments, the human relaxin-2 A chain derivatives comprise or consist of SEQ ID NO: 558.

In some embodiments, the human relaxin-2 A chain derivatives comprise or consist of SEQ ID NO: 8, wherein the amino acid at position 9 is lysine (K), glutamine (Q), aspartic acid (D), glutamic acid (E), leucine (L), isoleucine (I), or tyrosine (Y); the amino acid at position 17 is is lysine (K), glutamine (Q), aspartic acid (D), glutamic acid (E), leucine (L), isoleucine (I), or tyrosine (Y); and the amino acid at position 18 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 9, wherein the amino acid at position 9 is lysine (K), glutamine (Q), aspartic acid (D), glutamic acid (E), leucine (L), isoleucine (I), or tyrosine (Y); the amino acid at position 17 is lysine (K), glutamine (Q), aspartic acid (D), glutamic acid (E), leucine (L), isoleucine (I), or tyrosine (Y); and the amino acid at position 18 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 10, wherein the amino acid at position 9 is lysine (K), glutamine (Q), aspartic acid (D), glutamic acid (E), leucine (L), isoleucine (I), or tyrosine (Y); the amino acid at position 17 is lysine (K), glutamine (Q), aspartic acid (D), glutamic acid (E), leucine (L), isoleucine (I), or tyrosine (Y); and the amino acid at position 18 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 11, wherein the amino acid at position 9 is lysine (K), glutamine (Q), aspartic acid (D), glutamic acid (E), leucine (L), isoleucine (I), or tyrosine (Y); the amino acid at position 17 is lysine (K), glutamine (Q), aspartic acid (D), glutamic acid (E), leucine (L), isoleucine (I), or tyrosine (Y); and the amino acid at position 18 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 12, wherein the amino acid at position 9 is lysine (K), glutamine (Q), aspartic acid (D), glutamic acid (E), leucine (L), isoleucine (I), or tyrosine (Y); the amino acid at position 17 is lysine (K), glutamine (Q), aspartic acid (D), glutamic acid (E), leucine (L), isoleucine (I), or tyrosine (Y); and the amino acid at position 18 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 13, wherein the amino acid at position 9 is lysine (K), glutamine (Q), aspartic acid (D), glutamic acid (E), leucine (L), isoleucine (I), or tyrosine (Y); the amino acid at position 17 is lysine (K), glutamine (Q), aspartic acid (D), glutamic acid (E), leucine (L), isoleucine (I), or tyrosine (Y); and the amino acid at position 18 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 14, wherein the amino acid at position 9 is lysine (K), glutamine (Q), aspartic acid (D), glutamic acid (E), leucine (L), isoleucine (I), or tyrosine (Y); the amino acid at position 17 is lysine (K), glutamine (Q), aspartic acid (D), glutamic acid (E), leucine (L), isoleucine (I), or tyrosine (Y); and the amino acid at position 18 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 15, wherein the amino acid at position 9 is lysine (K), glutamine (Q), aspartic acid (D), glutamic acid (E), leucine (L), isoleucine (I), or tyrosine (Y); the amino acid at position 17 is lysine (K), glutamine (Q), aspartic acid (D), glutamic acid (E), leucine (L), isoleucine (I), or tyrosine (Y); and the amino acid at position 18 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 257, wherein the amino acid at position 9 is lysine (K), glutamine (Q), aspartic acid (D), glutamic acid (E), leucine (L), isoleucine (I), or tyrosine (Y); the amino acid at position 17 is lysine (K), glutamine (Q), aspartic acid (D), glutamic acid (E), leucine (L), isoleucine (I), or tyrosine (Y); and the amino acid at position 18 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 259, wherein the amino acid at position 10 is lysine (K), glutamine (Q), aspartic acid (D), glutamic acid (E), leucine (L), isoleucine (I), or tyrosine (Y); the amino acid at position 18 is lysine (K), glutamine (Q), aspartic acid (D), glutamic acid (E), leucine (L), isoleucine (I), or tyrosine (Y); and the amino acid at position 19 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 260, wherein the amino acid at position 10 is lysine (K), glutamine (Q), aspartic acid (D), glutamic acid (E), leucine (L), isoleucine (I), or tyrosine (Y); the amino acid at position 18 is lysine (K), glutamine (Q), aspartic acid (D), glutamic acid (E), leucine (L), isoleucine (I), or tyrosine (Y); and the amino acid at position 19 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 261, wherein the amino acid at position 10 is lysine (K), glutamine (Q), aspartic acid (D), glutamic acid (E), leucine (L), isoleucine (I), or tyrosine (Y); the amino acid at position 18 is lysine (K), glutamine (Q), aspartic acid (D), glutamic acid (E), leucine (L), isoleucine (I), or tyrosine (Y); and the amino acid at position 19 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 503, wherein the amino acid at position 1 is lysine (K), glutamine (Q), aspartic acid (D), glutamic acid (E), leucine (L), isoleucine (I), or tyrosine (Y); the amino acid at position 9 is lysine (K), glutamine (Q), aspartic acid (D), glutamic acid (E), leucine (L), isoleucine (I), or tyrosine (Y); and the amino acid at position 10 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 504, wherein the amino acid at position 1 is lysine (K), glutamine (Q), aspartic acid (D), glutamic acid (E), leucine (L), isoleucine (I), or tyrosine (Y); the amino acid at position 9 is lysine (K), glutamine (Q), aspartic acid (D), glutamic acid (E), leucine (L), isoleucine (I), or tyrosine (Y); and the amino acid at position 10 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 507, wherein the amino acid at position 9 is lysine (K), glutamine (Q), aspartic acid (D), glutamic acid (E), leucine (L), isoleucine (I), or tyrosine (Y); the amino acid at position 17 is lysine (K), glutamine (Q), aspartic acid (D), glutamic acid (E), leucine (L), isoleucine (I), or tyrosine (Y); and the amino acid at position 18 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 550, wherein the amino acid at position 9 is lysine (K), glutamine (Q), aspartic acid (D), glutamic acid (E), leucine (L), isoleucine (I), or tyrosine (Y); the amino acid at position 17 is lysine (K), glutamine (Q), aspartic acid (D), glutamic acid (E), leucine (L), isoleucine (I), or tyrosine (Y); and the amino acid at position 18 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 550, wherein the amino acid at position 10 is lysine (K), glutamine (Q), aspartic acid (D), glutamic acid (E), leucine (L), isoleucine (I), or tyrosine (Y); the amino acid at position 18 is lysine (K), glutamine (Q), aspartic acid (D), glutamic acid (E), leucine (L), isoleucine (I), or tyrosine (Y); and the amino acid at position 19 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 551, wherein the amino acid at position 10 is lysine (K), glutamine (Q), aspartic acid (D), glutamic acid (E), leucine (L), isoleucine (I), or tyrosine (Y); the amino acid at position 18 is lysine (K), glutamine (Q), aspartic acid (D), glutamic acid (E), leucine (L), isoleucine (I), or tyrosine (Y); and the amino acid at position 19 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 553, wherein the amino acid at position 10 is lysine (K), glutamine (Q), aspartic acid (D), glutamic acid (E), leucine (L), isoleucine (I), or tyrosine (Y); the amino acid at position 18 is lysine (K), glutamine (Q), aspartic acid (D), glutamic acid (E), leucine (L), isoleucine (I), or tyrosine (Y); and the amino acid at position 19 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 554, wherein the amino acid at position 10 is lysine (K), glutamine (Q), aspartic acid (D), glutamic acid (E), leucine (L), isoleucine (I), or tyrosine (Y); the amino acid at position 18 is lysine (K), glutamine (Q), aspartic acid (D), glutamic acid (E), leucine (L), isoleucine (I), or tyrosine (Y); and the amino acid at position 19 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 555, wherein the amino acid at position 10 is lysine (K), glutamine (Q), aspartic acid (D), glutamic acid (E), leucine (L), isoleucine (I), or tyrosine (Y); the amino acid at position 18 is lysine (K), glutamine (Q), aspartic acid (D), glutamic acid (E), leucine (L), isoleucine (I), or tyrosine (Y); and the amino acid at position 19 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 556, wherein the amino acid at position 10 is lysine (K), glutamine (Q), aspartic acid (D), glutamic acid (E), leucine (L), isoleucine (I), or tyrosine (Y); the amino acid at position 18 is lysine (K), glutamine (Q), aspartic acid (D), glutamic acid (E), leucine (L), isoleucine (I), or tyrosine (Y); and the amino acid at position 19 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 557, wherein the amino acid at position 10 is lysine (K), glutamine (Q), aspartic acid (D), glutamic acid (E), leucine (L), isoleucine (I), or tyrosine (Y); the amino acid at position 18 is lysine (K), glutamine (Q), aspartic acid (D), glutamic acid (E), leucine (L), isoleucine (I), or tyrosine (Y); and the amino acid at position 19 is glutamine (Q), aspartic acid (D), glutamic acid (E), leucine (L), isoleucine (I), tyrosine (Y), or arginine (R). In some embodiments, the human relaxin-2 A chain derivatives comprise or consist of SEQ ID NO: 558, wherein the amino acid at position 10 is lysine (K), glutamine (Q), aspartic acid (D), glutamic acid (E), leucine (L), isoleucine (I), or tyrosine (Y); the amino acid at position 18 is lysine (K), glutamine (Q), aspartic acid (D), glutamic acid (E), leucine (L), isoleucine (I), or tyrosine (Y); and the amino acid at position 19 is glutamine (Q), aspartic acid (D), glutamic acid (E), leucine (L), isoleucine (I), tyrosine (Y), or arginine (R).

In some embodiments, the human relaxin-2 A chain derivatives further comprise one or more substitutions that improve the stability of the relaxin-2 A chain derivatives, e.g., stability of the relaxin-2 A chain derivatives after light or heat exposure, as measured by methods known in the art, e.g., SEC to assess aggregate formation, and CE-SDS to assess purity. In some embodiments, the amino acid in the human relaxin-2 A chain derivatives corresponding to amino acid position 12 in SEQ ID NO: 12 is a glutamine (Q).

### Linker Peptides

The disclosure provides linker peptides, wherein the peptides have at least two acidic amino acids. In some embodiments, the acidic amino acid is glutamate. In some embodiments, the acidic amino acid is aspartate. In some embodiments, the acidic amino acid is a non-standard amino acid. In some embodiments, the acidic amino acid is 2-aminoadipic acid, 2-aminobutyric acid or 2-aminopimelic acid. In some embodiments, the linker peptide has 2, 3, 4, 5, 6, 7, 8, 9, or 10 acidic amino acids.

In some embodiments, the linker peptide is 8, 9, 10, 11, 12, 13, 14, or 15 amino acids in length. In some embodiments, the linker peptide is 12, 13, 14, or 15 amino acids in length. In some embodiments, the linker peptide has 2, 3, 4, or 5 acidic amino acids. In some embodiments, the linker peptide is 12, 13, 14, or 15 amino acids in length and has 2, 3, 4, or 5 acidic amino acids. In some embodiments, the remaining amino acids are non-acidic amino acids. In some embodiments, the non-acidic amino acids can be any standard amino acid that is not aspartate or glutamate. In some embodiments, non-acidic amino acids can be any amino acid that does not have a carboxylic acid in its side chain. In some embodiments, the non-acidic amino acid is glycine, proline, serine, arginine, histidine, lysine, threonine, asparagine, glutamine, cysteine, alanine, valine, isoleucine, leucine, methionine, phenylalanine, tyrosine, or tryptophan. In some embodiments, the non-acidic amino acid is glycine, proline, or cysteine. In some embodiments, the non-acidic amino acid is glycine.

In some embodiments, the linker peptide comprises acidic amino acids, wherein all the acidic amino acids are the same amino acids. In some embodiments, the acidic amino acids in the linker peptide are both/all glutamates. In some embodiments, the acidic amino acids in the linker peptide are both/all aspartates. In some embodiments, the linker peptide comprises amino acids that are a mixture of acidic amino acids. In some embodiments, the linker peptide comprises both glutamate and aspartate as acidic amino acids.

In some embodiments, the linker peptide comprises an amino acid sequence selected from the group consisting of
X₁₇X₁₇X₁₇X₁₈X₁₇X₁₇X₁₇X₁₈X₁₇X₁₇X₁₇X₁₈X₁₇;
X₁₇X₁₇X₁₇X₁₈X₁₇X₁₇X₁₇X₁₈X₁₇X₁₇X₁₇X₁₈X₁₇X₁₇X₁₇;
X₁₇X₁₇X₁₈X₁₇X₁₇X₁₇X₁₈X₁₈X₁₇X₁₇X₁₇X₁₈X₁₇X₁₇;
X₁₇X₁₇X₁₇X₁₈X₁₈X₁₇X₁₇X₁₇X₁₈X₁₈X₁₇X₁₇X₁₇; and
X₁₇X₁₇X₁₈X₁₇X₁₈X₁₇X₁₇X₁₈X₁₇X₁₈X₁₇X₁₇X₁₇,
   wherein X₁₇ is a non-acidic amino acid and X₁₈ is an acidic amino acid.

In some embodiments, the linker peptide comprises non-acidic amino acids, wherein all the non-acidic amino acids are the same amino acids. In some embodiments, the non-acidic amino acids in the linker peptide are all glycine. In some embodiments, the linker peptide comprises amino acids that are a mixture of non-acidic amino acids. In some embodiments, the linker peptide comprises 2, 3, 4, 5, 6, 7, 8, 9, or 10 different types of non-acidic amino acids.

In some embodiments, the linker peptide comprises the amino acid sequence ASDAAGAX₈AX₉AGA (SEQ ID NO: 17), wherein X₈ is aspartic acid (D), glutamic acid (E), asparagine (N), or glutamine (Q); and X₉ is aspartic acid (D), glutamic acid (E), asparagine (N), or glutamine (Q). In some embodiments, X₈ is aspartic acid (D), glutamic acid (E), asparagine (N), or glutamine (Q), and X₉ is aspartic acid (D), glutamic acid (E), or glutamine (Q). In some embodiments, X₈ is aspartic acid (D), glutamic acid (E), or glutamine (Q), and X₉ is aspartic acid (D), glutamic acid (E), asparagine (N), or glutamine (Q).

In some embodiments, the linker peptide comprises GGEGSGGEGX₁₀GGG (SEQ ID NO: 25), wherein X₁₀ is glutamic acid (E) or serine(S).

In some embodiments, the linker peptide comprises or consists of the amino acid sequences shown in Table 3, below.

TABLE 3
Linker Peptide Sequences

| SEQ ID NO: | Amino Acid Sequence |
|---|---|
| 18 | ASDAAGADADAGA |
| 19 | ASDAAGADANAGA |
| 20 | ASDAAGADAQAGA |
| 21 | ASDAAGAEAEAGA |
| 22 | ASDAAGANADAGA |
| 23 | ASDAAGAQADAGA |
| 24 | ASDAAGAQAQAGA |
| 26 | GGEGSGGEGEGGG |
| 27 | GGEGSGGEGSGGG |
| 267 | GGGE |
| 268 | GEGE |
| 269 | GGEG |
| 270 | GEGG |
| 271 | GGEE |
| 272 | GGGEGGGEGGGEG |
| 273 | GGGEGGGEGGGEGGG |
| 274 | GEGGGEEGGGEGG |
| 275 | GGGEEGGGEEGGG |
| 276 | GGEGEGGEGEGGS |

In some embodiments, the linker peptide comprises 2, 3, 4, or 5 repeats of SEQ ID NO: 267, 268, 269, 270, or 271. For example, 3 repeats of SEQ ID NO: 267 would be the amino acid sequence of GGGEGGGEGGGE (SEQ ID NO: 277).

In some embodiments, the linker peptide comprises or consists of SEQ ID NO: 18. In some embodiments, the linker peptide comprises or consists of SEQ ID NO: 19. In some embodiments, the linker peptide comprises or consists of SEQ ID NO: 20. In some embodiments, the linker peptide comprises or consists of SEQ ID NO: 21. In some embodiments, the linker peptide comprises or consists of SEQ ID NO: 22. In some embodiments, the linker peptide comprises or consists of SEQ ID NO: 23. In some embodiments, the linker peptide comprises or consists of SEQ ID NO: 24. In some embodiments, the linker peptide comprises or consists of SEQ ID NO: 26. In some embodiments, the linker peptide comprises or consists of SEQ ID NO: 27. In some embodiments, the linker peptide comprises or consists of SEQ ID NO: 267. In some embodiments, the linker peptide comprises or consists of SEQ ID NO: 268. In some embodiments, the linker peptide comprises or consists of SEQ ID NO: 269. In some embodiments, the linker peptide comprises or consists of SEQ ID NO: 270. In some embodiments, the linker peptide comprises or consists of SEQ ID NO: 271. In some embodiments, the linker peptide comprises or consists of SEQ ID NO: 272. In some embodiments, the linker peptide comprises or consists of SEQ ID NO: 273. In some embodiments, the linker peptide comprises or consists of SEQ ID NO: 274. In some embodiments, the linker peptide comprises or consists of SEQ ID NO: 275. In some embodiments, the linker peptide comprises or consists of SEQ ID NO: 276. In some embodiments, the linker peptide comprises or consists of SEQ ID NO: 277.

### Relaxin/Linker Peptide Combinations for the Fusion Protein

In some embodiments, the fusion protein comprises an N-terminal or first peptide, a linker peptide, and a C-terminal or second peptide. In some embodiments, the N-terminal peptide comprises a human relaxin-2 A chain or a derivative thereof (RelA) and the C-terminal peptide comprises a human relaxin-2 B chain or a derivative thereof (RelB). In some embodiments, the N-terminal peptide comprises a human relaxin-2 B chain or a derivative thereof and the C-terminal peptide comprises a human relaxin-2 A chain or a derivative thereof. Any combination of any of the embodiments of the human relaxin-2 A chain or a derivative thereof, with a human relaxin-2 A chain or a derivative thereof linked by any of the linker peptides disclosed herein can be used to construct embodiments of the fusion proteins described herein. In some embodiments, at least one of the N-terminal peptide and the C-terminal peptide is a derivative of a human relaxin-2 A chain or a human relaxin-2 B chain. In some embodiments, the N-terminal peptide comprises a human relaxin-2 A chain derivative and the C-terminal peptide comprises a human relaxin-2 B chain derivative. In some embodiments, the N-terminal peptide comprises a human relaxin-2 B chain derivative and the C-terminal peptide comprises a human relaxin-2 A chain derivative.

In some embodiments, the human relaxin-2 B chain derivative consists of 15 amino acids and the human relaxin-2 A chain derivative consists of 16-25 amino acids. In some embodiments, the human relaxin-2 B chain derivative consists of 16 amino acids and the human relaxin-2 A chain derivative consists of 16-25 amino acids. In some embodiments, the human relaxin-2 B chain derivative consists of 17 amino acids and the human relaxin-2 A chain derivative consists of 16-25 amino acids. In some embodiments, the human relaxin-2 B chain derivative consists of 18 amino acids and the human relaxin-2 A chain derivative consists of 16-25 amino acids. In some embodiments, the human relaxin-2 B chain derivative consists of 19 amino acids and the human relaxin-2 A chain derivative consists of 16-25 amino acids. In some embodiments, the human relaxin-2 B chain derivative consists of 20 amino acids and the human relaxin-2 A chain derivative consists of 16-25 amino acids. In some embodiments, the human relaxin-2 B chain derivative consists of 21 amino acids and the human relaxin-2 A chain derivative consists of 16-25 amino acids. In some embodiments, the human relaxin-2 B chain derivative consists of 22 amino acids and the human relaxin-2 A chain derivative consists of 16-25 amino acids. In some embodiments, the human relaxin-2 B chain derivative consists of 23 amino acids and the human relaxin-2 A chain derivative consists of 16-25 amino acids. In some embodiments, the human relaxin-2 B chain derivative consists of 24 amino acids and the human relaxin-2 A chain derivative consists of 16-25 amino acids. In some embodiments, the human relaxin-2 B chain derivative consists of 25 amino acids and the human relaxin-2 A chain derivative consists of 16-25 amino acids. In some embodiments, the human relaxin-2 B chain derivative consists of 26 amino acids and the human relaxin-2 A chain derivative consists of 16-25 amino acids. In some embodiments, the human relaxin-2 B chain derivative consists of 27 amino acids and the human relaxin-2 A chain derivative consists of 16-25 amino acids. In some embodiments, the human relaxin-2 B chain derivative consists of 28 amino acids and the human relaxin-2 A chain derivative consists of 16-25 amino acids. In some embodiments, the human relaxin-2 B chain derivative consists of 29 amino acids and the human relaxin-2 A chain derivative consists of 16-25 amino acids.

In some embodiments, the human relaxin-2 B chain derivative consists of 15-29 amino acids and the human relaxin-2 A chain derivative consists of 16 amino acids. In some embodiments, the human relaxin-2 B chain derivative consists of 15-29 amino acids and the human relaxin-2 A chain derivative consists of 17 amino acids. In some embodiments, the human relaxin-2 B chain derivative consists of 15-29 amino acids and the human relaxin-2 A chain derivative consists of 18 amino acids. In some embodiments, the human relaxin-2 B chain derivative consists of 15-29 amino acids and the human relaxin-2 A chain derivative consists of 19 amino acids. In some embodiments, the human relaxin-2 B chain derivative consists of 15-29 amino acids and the human relaxin-2 A chain derivative consists of 20 amino acids. In some embodiments, the human relaxin-2 B chain derivative consists of 15-29 amino acids and the human relaxin-2 A chain derivative consists of 21 amino acids. In some embodiments, the human relaxin-2 B chain derivative consists of 15-29 amino acids and the human relaxin-2 A chain derivative consists of 22 amino acids. In some embodiments, the human relaxin-2 B chain derivative consists of 15-29 amino acids and the human relaxin-2 A chain derivative consists of 23 amino acids. In some embodiments, the human relaxin-2 B chain derivative consists of 15-29 amino acids and the human relaxin-2 A chain derivative consists of 24 amino acids. In some embodiments, the human relaxin-2 B chain derivative consists of 15-29 amino acids and the human relaxin-2 A chain derivative consists of 25 amino acids.

Specific embodiments of the fusion proteins provided in this disclosure are shown below in Table 4.

TABLE 4
Fusion Proteins

| N-terminal | Linker | C-terminal | N-terminal | Linker | C-terminal |
|---|---|---|---|---|---|
| Peptide | peptide | Peptide | Peptide | peptide | Peptide |
| SEQ ID NO: | | | SEQ ID NO: | | |
| RelB-linker | | | RelB-linker | | |
| (SEQ ID NO: 18)-RelA | | | (SEQ ID NO: 19)-RelA | | |
| 1 | 18 | 8 | 1 | 19 | 8 |
| 2 | 18 | 8 | 2 | 19 | 8 |
| 3 | 18 | 8 | 3 | 19 | 8 |
| 4 | 18 | 8 | 4 | 19 | 8 |
| 5 | 18 | 8 | 5 | 19 | 8 |
| 6 | 18 | 8 | 6 | 19 | 8 |
| 249 | 18 | 8 | 249 | 19 | 8 |
| 255 | 18 | 8 | 255 | 19 | 8 |
| 256 | 18 | 8 | 256 | 19 | 8 |
| 502 | 18 | 8 | 502 | 19 | 8 |
| 1 | 18 | 9 | 1 | 19 | 9 |
| 2 | 18 | 9 | 2 | 19 | 9 |
| 3 | 18 | 9 | 3 | 19 | 9 |
| 4 | 18 | 9 | 4 | 19 | 9 |
| 5 | 18 | 9 | 5 | 19 | 9 |
| 6 | 18 | 9 | 6 | 19 | 9 |
| 249 | 18 | 9 | 249 | 19 | 9 |
| 255 | 18 | 9 | 255 | 19 | 9 |
| 256 | 18 | 9 | 256 | 19 | 9 |
| 502 | 18 | 9 | 502 | 19 | 9 |
| 1 | 18 | 10 | 1 | 19 | 10 |
| 2 | 18 | 10 | 2 | 19 | 10 |
| 3 | 18 | 10 | 3 | 19 | 10 |
| 4 | 18 | 10 | 4 | 19 | 10 |
| 5 | 18 | 10 | 5 | 19 | 10 |
| 6 | 18 | 10 | 6 | 19 | 10 |
| 249 | 18 | 10 | 249 | 19 | 10 |
| 255 | 18 | 10 | 255 | 19 | 10 |
| 256 | 18 | 10 | 256 | 19 | 10 |
| 502 | 18 | 10 | 502 | 19 | 10 |
| 1 | 18 | 11 | 1 | 19 | 11 |
| 2 | 18 | 11 | 2 | 19 | 11 |
| 3 | 18 | 11 | 3 | 19 | 11 |
| 4 | 18 | 11 | 4 | 19 | 11 |
| 5 | 18 | 11 | 5 | 19 | 11 |
| 6 | 18 | 11 | 6 | 19 | 11 |
| 249 | 18 | 11 | 249 | 19 | 11 |
| 255 | 18 | 11 | 255 | 19 | 11 |
| 256 | 18 | 11 | 256 | 19 | 11 |
| 502 | 18 | 11 | 502 | 19 | 11 |
| 1 | 18 | 12 | 1 | 19 | 12 |
| 2 | 18 | 12 | 2 | 19 | 12 |
| 3 | 18 | 12 | 3 | 19 | 12 |
| 4 | 18 | 12 | 4 | 19 | 12 |
| 5 | 18 | 12 | 5 | 19 | 12 |
| 6 | 18 | 12 | 6 | 19 | 12 |
| 249 | 18 | 12 | 249 | 19 | 12 |
| 255 | 18 | 12 | 255 | 19 | 12 |
| 256 | 18 | 12 | 256 | 19 | 12 |
| 502 | 18 | 12 | 502 | 19 | 12 |
| 1 | 18 | 13 | 1 | 19 | 13 |
| 2 | 18 | 13 | 2 | 19 | 13 |
| 3 | 18 | 13 | 3 | 19 | 13 |
| 4 | 18 | 13 | 4 | 19 | 13 |
| 5 | 18 | 13 | 5 | 19 | 13 |
| 6 | 18 | 13 | 6 | 19 | 13 |
| 249 | 18 | 13 | 249 | 19 | 13 |
| 255 | 18 | 13 | 255 | 19 | 13 |
| 256 | 18 | 13 | 256 | 19 | 13 |
| 502 | 18 | 13 | 502 | 19 | 13 |
| 1 | 18 | 14 | 1 | 19 | 14 |
| 2 | 18 | 14 | 2 | 19 | 14 |
| 3 | 18 | 14 | 3 | 19 | 14 |
| 4 | 18 | 14 | 4 | 19 | 14 |
| 5 | 18 | 14 | 5 | 19 | 14 |
| 6 | 18 | 14 | 6 | 19 | 14 |
| 249 | 18 | 14 | 249 | 19 | 14 |
| 255 | 18 | 14 | 255 | 19 | 14 |
| 256 | 18 | 14 | 256 | 19 | 14 |
| 502 | 18 | 14 | 502 | 19 | 14 |
| 1 | 18 | 15 | 1 | 19 | 15 |
| 2 | 18 | 15 | 2 | 19 | 15 |
| 3 | 18 | 15 | 3 | 19 | 15 |
| 4 | 18 | 15 | 4 | 19 | 15 |
| 5 | 18 | 15 | 5 | 19 | 15 |
| 6 | 18 | 15 | 6 | 19 | 15 |
| 249 | 18 | 15 | 249 | 19 | 15 |
| 255 | 18 | 15 | 255 | 19 | 15 |
| 256 | 18 | 15 | 256 | 19 | 15 |
| 502 | 18 | 15 | 502 | 19 | 15 |
| 1 | 18 | 257 | 1 | 19 | 257 |
| 2 | 18 | 257 | 2 | 19 | 257 |
| 3 | 18 | 257 | 3 | 19 | 257 |
| 4 | 18 | 257 | 4 | 19 | 257 |
| 5 | 18 | 257 | 5 | 19 | 257 |
| 6 | 18 | 257 | 6 | 19 | 257 |
| 249 | 18 | 257 | 249 | 19 | 257 |
| 255 | 18 | 257 | 255 | 19 | 257 |
| 256 | 18 | 257 | 256 | 19 | 257 |
| 502 | 18 | 257 | 502 | 19 | 257 |
| 1 | 18 | 259 | 1 | 19 | 259 |
| 2 | 18 | 259 | 2 | 19 | 259 |
| 3 | 18 | 259 | 3 | 19 | 259 |
| 4 | 18 | 259 | 4 | 19 | 259 |
| 5 | 18 | 259 | 5 | 19 | 259 |
| 6 | 18 | 259 | 6 | 19 | 259 |
| 249 | 18 | 259 | 249 | 19 | 259 |
| 255 | 18 | 259 | 255 | 19 | 259 |
| 256 | 18 | 259 | 256 | 19 | 259 |
| 502 | 18 | 259 | 502 | 19 | 259 |
| 1 | 18 | 260 | 1 | 19 | 260 |
| 2 | 18 | 260 | 2 | 19 | 260 |
| 3 | 18 | 260 | 3 | 19 | 260 |
| 4 | 18 | 260 | 4 | 19 | 260 |
| 5 | 18 | 260 | 5 | 19 | 260 |
| 6 | 18 | 260 | 6 | 19 | 260 |
| 249 | 18 | 260 | 249 | 19 | 260 |
| 255 | 18 | 260 | 255 | 19 | 260 |
| 256 | 18 | 260 | 256 | 19 | 260 |
| 502 | 18 | 260 | 502 | 19 | 260 |
| 1 | 18 | 261 | 1 | 19 | 26 |
| 2 | 18 | 261 | 2 | 19 | 261 |
| 3 | 18 | 261 | 3 | 19 | 261 |
| 4 | 18 | 26 | 4 | 19 | 261 |
| 5 | 18 | 261 | 5 | 19 | 261 |
| 6 | 18 | 26 | 6 | 19 | 261 |
| 249 | 18 | 26 | 249 | 19 | 261 |
| 255 | 18 | 261 | 255 | 19 | 261 |
| 256 | 18 | 261 | 256 | 19 | 261 |
| 502 | 18 | 26 | 502 | 19 | 261 |
| 1 | 18 | 503 | 1 | 19 | 503 |
| 2 | 18 | 503 | 2 | 19 | 503 |
| 3 | 18 | 503 | 3 | 19 | 503 |
| 4 | 18 | 503 | 4 | 19 | 503 |
| 5 | 18 | 503 | 5 | 19 | 503 |
| 6 | 18 | 503 | 6 | 19 | 503 |
| 249 | 18 | 503 | 249 | 19 | 503 |
| 255 | 18 | 503 | 255 | 19 | 503 |
| 256 | 18 | 503 | 256 | 19 | 503 |
| 502 | 18 | 503 | 502 | 19 | 503 |
| 1 | 18 | 504 | 1 | 19 | 504 |
| 2 | 18 | 504 | 2 | 19 | 504 |
| 3 | 18 | 504 | 3 | 19 | 504 |
| 4 | 18 | 504 | 4 | 19 | 504 |
| 5 | 18 | 504 | 5 | 19 | 504 |
| 6 | 18 | 504 | 6 | 19 | 504 |
| 249 | 18 | 504 | 249 | 19 | 504 |
| 255 | 18 | 504 | 255 | 19 | 504 |
| 256 | 18 | 504 | 256 | 19 | 504 |
| 502 | 18 | 504 | 502 | 19 | 504 |
| 1 | 18 | 507 | 1 | 19 | 507 |
| 2 | 18 | 507 | 2 | 19 | 507 |
| 3 | 18 | 507 | 3 | 19 | 507 |
| 4 | 18 | 507 | 4 | 19 | 507 |
| 5 | 18 | 507 | 5 | 19 | 507 |
| 6 | 18 | 507 | 6 | 19 | 507 |
| 249 | 18 | 507 | 249 | 19 | 507 |
| 255 | 18 | 507 | 255 | 19 | 507 |
| 256 | 18 | 507 | 256 | 19 | 507 |
| 502 | 18 | 507 | 502 | 19 | 507 |
| 1 | 18 | 550 | 1 | 19 | 550 |
| 2 | 18 | 550 | 2 | 19 | 550 |
| 3 | 18 | 550 | 3 | 19 | 550 |
| 4 | 18 | 550 | 4 | 19 | 550 |
| 5 | 18 | 550 | 5 | 19 | 550 |
| 6 | 18 | 550 | 6 | 19 | 550 |
| 249 | 18 | 550 | 249 | 19 | 550 |
| 255 | 18 | 550 | 255 | 19 | 550 |
| 256 | 18 | 550 | 256 | 19 | 550 |
| 502 | 18 | 550 | 502 | 19 | 550 |
| 1 | 18 | 551 | 1 | 19 | 551 |
| 2 | 18 | 551 | 2 | 19 | 551 |
| 3 | 18 | 551 | 3 | 19 | 551 |
| 4 | 18 | 551 | 4 | 19 | 551 |
| 5 | 18 | 551 | 5 | 19 | 551 |
| 6 | 18 | 551 | 6 | 19 | 551 |
| 249 | 18 | 551 | 249 | 19 | 551 |
| 255 | 18 | 551 | 255 | 19 | 551 |
| 256 | 18 | 551 | 256 | 19 | 551 |
| 502 | 18 | 551 | 502 | 19 | 551 |
| 1 | 18 | 552 | 1 | 19 | 552 |
| 2 | 18 | 552 | 2 | 19 | 552 |
| 3 | 18 | 552 | 3 | 19 | 552 |
| 4 | 18 | 552 | 4 | 19 | 552 |
| 5 | 18 | 552 | 5 | 19 | 552 |
| 6 | 18 | 552 | 6 | 19 | 552 |
| 249 | 18 | 552 | 249 | 19 | 552 |
| 255 | 18 | 552 | 255 | 19 | 552 |
| 256 | 18 | 552 | 256 | 19 | 552 |
| 502 | 18 | 552 | 502 | 19 | 552 |
| 1 | 18 | 553 | 1 | 19 | 553 |
| 2 | 18 | 553 | 2 | 19 | 553 |
| 3 | 18 | 553 | 3 | 19 | 553 |
| 4 | 18 | 553 | 4 | 19 | 553 |
| 5 | 18 | 553 | 5 | 19 | 553 |
| 6 | 18 | 553 | 6 | 19 | 553 |
| 249 | 18 | 553 | 249 | 19 | 553 |
| 255 | 18 | 553 | 255 | 19 | 553 |
| 256 | 18 | 553 | 256 | 19 | 553 |
| 502 | 18 | 553 | 502 | 19 | 553 |
| 1 | 18 | 554 | 1 | 19 | 554 |
| 2 | 18 | 554 | 2 | 19 | 554 |
| 3 | 18 | 554 | 3 | 19 | 554 |
| 4 | 18 | 554 | 4 | 19 | 554 |
| 5 | 18 | 554 | 5 | 19 | 554 |
| 6 | 18 | 554 | 6 | 19 | 554 |
| 249 | 18 | 554 | 249 | 19 | 554 |
| 255 | 18 | 554 | 255 | 19 | 554 |
| 256 | 18 | 554 | 256 | 19 | 554 |
| 502 | 18 | 554 | 502 | 19 | 554 |
| 1 | 18 | 555 | 1 | 19 | 555 |
| 2 | 18 | 555 | 2 | 19 | 555 |
| 3 | 18 | 555 | 3 | 19 | 555 |
| 4 | 18 | 555 | 4 | 19 | 555 |
| 5 | 18 | 555 | 5 | 19 | 555 |
| 6 | 18 | 555 | 6 | 19 | 555 |
| 249 | 18 | 555 | 249 | 19 | 555 |
| 255 | 18 | 555 | 255 | 19 | 555 |
| 256 | 18 | 555 | 256 | 19 | 555 |
| 502 | 18 | 555 | 502 | 19 | 555 |
| 1 | 18 | 556 | 1 | 19 | 556 |
| 2 | 18 | 556 | 2 | 19 | 556 |
| 3 | 18 | 556 | 3 | 19 | 556 |
| 4 | 18 | 556 | 4 | 19 | 556 |
| 5 | 18 | 556 | 5 | 19 | 556 |
| 6 | 18 | 556 | 6 | 19 | 556 |
| 249 | 18 | 556 | 249 | 19 | 556 |
| 255 | 18 | 556 | 255 | 19 | 556 |
| 256 | 18 | 556 | 256 | 19 | 556 |
| 502 | 18 | 556 | 502 | 19 | 556 |
| 1 | 18 | 557 | 1 | 19 | 557 |
| 2 | 18 | 557 | 2 | 19 | 557 |
| 3 | 18 | 557 | 3 | 19 | 557 |
| 4 | 18 | 557 | 4 | 19 | 557 |
| 5 | 18 | 557 | 5 | 19 | 557 |
| 6 | 18 | 557 | 6 | 19 | 557 |
| 249 | 18 | 557 | 249 | 19 | 557 |
| 255 | 18 | 557 | 255 | 19 | 557 |
| 256 | 18 | 557 | 256 | 19 | 557 |
| 502 | 18 | 557 | 502 | 19 | 557 |
| 1 | 18 | 558 | 1 | 19 | 558 |
| 2 | 18 | 558 | 2 | 19 | 558 |
| 3 | 18 | 558 | 3 | 19 | 558 |
| 4 | 18 | 558 | 4 | 19 | 558 |
| 5 | 18 | 558 | 5 | 19 | 558 |
| 6 | 18 | 558 | 6 | 19 | 558 |
| 249 | 18 | 558 | 249 | 19 | 558 |
| 255 | 18 | 558 | 255 | 19 | 558 |
| 256 | 18 | 558 | 256 | 19 | 558 |
| 502 | 18 | 558 | 502 | 19 | 558 |
| RelB-linker | | | RelB-linker | | |
| (SEQ ID NO: 20)-RelA | | | (SEQ ID NO: 21)-RelA | | |
| 1 | 20 | 8 | 1 | 21 | 8 |
| 2 | 20 | 8 | 2 | 21 | 8 |
| 3 | 20 | 8 | 3 | 21 | 8 |
| 4 | 20 | 8 | 4 | 21 | 8 |
| 5 | 20 | 8 | 5 | 21 | 8 |
| 6 | 20 | 8 | 6 | 21 | 8 |
| 249 | 20 | 8 | 249 | 21 | 8 |
| 255 | 20 | 8 | 255 | 21 | 8 |
| 256 | 20 | 8 | 256 | 21 | 8 |
| 502 | 20 | 8 | 502 | 21 | 8 |
| 1 | 20 | 9 | 1 | 21 | 9 |
| 2 | 20 | 9 | 2 | 21 | 9 |
| 3 | 20 | 9 | 3 | 21 | 9 |
| 4 | 20 | 9 | 4 | 21 | 9 |
| 5 | 20 | 9 | 5 | 21 | 9 |
| 6 | 20 | 9 | 6 | 21 | 9 |
| 249 | 20 | 9 | 249 | 21 | 9 |
| 255 | 20 | 9 | 255 | 21 | 9 |
| 256 | 20 | 9 | 256 | 21 | 9 |
| 502 | 20 | 9 | 502 | 21 | 9 |
| 1 | 20 | 10 | 1 | 21 | 10 |
| 2 | 20 | 10 | 2 | 21 | 10 |
| 3 | 20 | 10 | 3 | 21 | 10 |
| 4 | 20 | 10 | 4 | 21 | 10 |
| 5 | 20 | 10 | 5 | 21 | 10 |
| 6 | 20 | 10 | 6 | 21 | 10 |
| 249 | 20 | 10 | 249 | 21 | 10 |
| 255 | 20 | 10 | 255 | 21 | 10 |
| 256 | 20 | 10 | 256 | 21 | 10 |
| 502 | 20 | 10 | 502 | 21 | 10 |
| 1 | 20 | 11 | 1 | 21 | 11 |
| 2 | 20 | 11 | 2 | 21 | 11 |
| 3 | 20 | 11 | 3 | 21 | 11 |
| 4 | 20 | 11 | 4 | 21 | 11 |
| 5 | 20 | 11 | 5 | 21 | 11 |
| 6 | 20 | 11 | 6 | 21 | 11 |
| 249 | 20 | 11 | 249 | 21 | 11 |
| 255 | 20 | 11 | 255 | 21 | 11 |
| 256 | 20 | 11 | 256 | 21 | 11 |
| 502 | 20 | 11 | 502 | 21 | 11 |
| 1 | 20 | 12 | 1 | 21 | 12 |
| 2 | 20 | 12 | 2 | 21 | 12 |
| 3 | 20 | 12 | 3 | 21 | 12 |
| 4 | 20 | 12 | 4 | 21 | 12 |
| 5 | 20 | 12 | 5 | 21 | 12 |
| 6 | 20 | 12 | 6 | 21 | 12 |
| 249 | 20 | 12 | 249 | 21 | 12 |
| 255 | 20 | 12 | 255 | 21 | 12 |
| 256 | 20 | 12 | 256 | 21 | 12 |
| 502 | 20 | 12 | 502 | 21 | 12 |
| 1 | 20 | 13 | 1 | 21 | 13 |
| 2 | 20 | 13 | 2 | 21 | 13 |
| 3 | 20 | 13 | 3 | 21 | 13 |
| 4 | 20 | 13 | 4 | 21 | 13 |
| 5 | 20 | 13 | 5 | 21 | 13 |
| 6 | 20 | 13 | 6 | 21 | 13 |
| 249 | 20 | 13 | 249 | 21 | 13 |
| 255 | 20 | 13 | 255 | 21 | 13 |
| 256 | 20 | 13 | 256 | 21 | 13 |
| 502 | 20 | 13 | 502 | 21 | 13 |
| 1 | 20 | 14 | 1 | 21 | 14 |
| 2 | 20 | 14 | 2 | 21 | 14 |
| 3 | 20 | 14 | 3 | 21 | 14 |
| 4 | 20 | 14 | 4 | 21 | 14 |
| 5 | 20 | 14 | 5 | 21 | 14 |
| 6 | 20 | 14 | 6 | 21 | 14 |
| 249 | 20 | 14 | 249 | 21 | 14 |
| 255 | 20 | 14 | 255 | 21 | 14 |
| 256 | 20 | 14 | 256 | 21 | 14 |
| 502 | 20 | 14 | 502 | 21 | 14 |
| 1 | 20 | 15 | 1 | 21 | 15 |
| 2 | 20 | 15 | 2 | 21 | 15 |
| 3 | 20 | 15 | 3 | 21 | 15 |
| 4 | 20 | 15 | 4 | 21 | 15 |
| 5 | 20 | 15 | 5 | 21 | 15 |
| 6 | 20 | 15 | 6 | 21 | 15 |
| 249 | 20 | 15 | 249 | 21 | 15 |
| 255 | 20 | 15 | 255 | 21 | 15 |
| 256 | 20 | 15 | 256 | 21 | 15 |
| 502 | 20 | 15 | 502 | 21 | 15 |
| 1 | 20 | 257 | 1 | 21 | 257 |
| 2 | 20 | 257 | 2 | 21 | 257 |
| 3 | 20 | 257 | 3 | 21 | 257 |
| 4 | 20 | 257 | 4 | 21 | 257 |
| 5 | 20 | 257 | 5 | 21 | 257 |
| 6 | 20 | 257 | 6 | 21 | 257 |
| 249 | 20 | 257 | 249 | 21 | 257 |
| 255 | 20 | 257 | 255 | 21 | 257 |
| 256 | 20 | 257 | 256 | 21 | 257 |
| 502 | 20 | 257 | 502 | 21 | 257 |
| 1 | 20 | 259 | 1 | 21 | 259 |
| 2 | 20 | 259 | 2 | 21 | 259 |
| 3 | 20 | 259 | 3 | 21 | 259 |
| 4 | 20 | 259 | 4 | 21 | 259 |
| 5 | 20 | 259 | 5 | 21 | 259 |
| 6 | 20 | 259 | 6 | 21 | 259 |
| 249 | 20 | 259 | 249 | 21 | 259 |
| 255 | 20 | 259 | 255 | 21 | 259 |
| 256 | 20 | 259 | 256 | 21 | 259 |
| 502 | 20 | 259 | 502 | 21 | 259 |
| 1 | 20 | 260 | 1 | 21 | 260 |
| 2 | 20 | 260 | 2 | 21 | 260 |
| 3 | 20 | 260 | 3 | 21 | 260 |
| 4 | 20 | 260 | 4 | 21 | 260 |
| 5 | 20 | 260 | 5 | 21 | 260 |
| 6 | 20 | 260 | 6 | 21 | 260 |
| 249 | 20 | 260 | 249 | 21 | 260 |
| 255 | 20 | 260 | 255 | 21 | 260 |
| 256 | 20 | 260 | 256 | 21 | 260 |
| 502 | 20 | 260 | 502 | 21 | 260 |
| 1 | 20 | 261 | 1 | 21 | 261 |
| 2 | 20 | 261 | 2 | 21 | 261 |
| 3 | 20 | 261 | 3 | 21 | 261 |
| 4 | 20 | 261 | 4 | 21 | 261 |
| 5 | 20 | 261 | 5 | 21 | 261 |
| 6 | 20 | 261 | 6 | 21 | 261 |
| 249 | 20 | 261 | 249 | 21 | 261 |
| 255 | 20 | 261 | 255 | 21 | 261 |
| 256 | 20 | 261 | 256 | 21 | 261 |
| 502 | 20 | 261 | 502 | 21 | 261 |
| 1 | 20 | 503 | 1 | 21 | 503 |
| 2 | 20 | 503 | 2 | 21 | 503 |
| 3 | 20 | 503 | 3 | 21 | 503 |
| 4 | 20 | 503 | 4 | 21 | 503 |
| 5 | 20 | 503 | 5 | 21 | 503 |
| 6 | 20 | 503 | 6 | 21 | 503 |
| 249 | 20 | 503 | 249 | 21 | 503 |
| 255 | 20 | 503 | 255 | 21 | 503 |
| 256 | 20 | 503 | 256 | 21 | 503 |
| 502 | 20 | 503 | 502 | 21 | 503 |
| 1 | 20 | 504 | 1 | 21 | 504 |
| 2 | 20 | 504 | 2 | 21 | 504 |
| 3 | 20 | 504 | 3 | 21 | 504 |
| 4 | 20 | 504 | 4 | 21 | 504 |
| 5 | 20 | 504 | 5 | 21 | 504 |
| 6 | 20 | 504 | 6 | 21 | 504 |
| 249 | 20 | 504 | 249 | 21 | 504 |
| 255 | 20 | 504 | 255 | 21 | 504 |
| 256 | 20 | 504 | 256 | 21 | 504 |
| 502 | 20 | 504 | 502 | 21 | 504 |
| 1 | 20 | 507 | 1 | 21 | 507 |
| 2 | 20 | 507 | 2 | 21 | 507 |
| 3 | 20 | 507 | 3 | 21 | 507 |
| 4 | 20 | 507 | 4 | 21 | 507 |
| 5 | 20 | 507 | 5 | 21 | 507 |
| 6 | 20 | 507 | 6 | 21 | 507 |
| 249 | 20 | 507 | 249 | 21 | 507 |
| 255 | 20 | 507 | 255 | 21 | 507 |
| 256 | 20 | 507 | 256 | 21 | 507 |
| 502 | 20 | 507 | 502 | 21 | 507 |
| 1 | 20 | 550 | 1 | 21 | 550 |
| 2 | 20 | 550 | 2 | 21 | 550 |
| 3 | 20 | 550 | 3 | 21 | 550 |
| 4 | 20 | 550 | 4 | 21 | 550 |
| 5 | 20 | 550 | 5 | 21 | 550 |
| 6 | 20 | 550 | 6 | 21 | 550 |
| 249 | 20 | 550 | 249 | 21 | 550 |
| 255 | 20 | 550 | 255 | 21 | 550 |
| 256 | 20 | 550 | 256 | 21 | 550 |
| 502 | 20 | 550 | 502 | 21 | 550 |
| 1 | 20 | 551 | 1 | 21 | 551 |
| 2 | 20 | 551 | 2 | 21 | 551 |
| 3 | 20 | 551 | 3 | 21 | 551 |
| 4 | 20 | 551 | 4 | 21 | 551 |
| 5 | 20 | 551 | 5 | 21 | 551 |
| 6 | 20 | 551 | 6 | 21 | 551 |
| 249 | 20 | 551 | 249 | 21 | 551 |
| 255 | 20 | 551 | 255 | 21 | 551 |
| 256 | 20 | 551 | 256 | 21 | 551 |
| 502 | 20 | 551 | 502 | 21 | 551 |
| 1 | 20 | 552 | 1 | 21 | 552 |
| 2 | 20 | 552 | 2 | 21 | 552 |
| 3 | 20 | 552 | 3 | 21 | 552 |
| 4 | 20 | 552 | 4 | 21 | 552 |
| 5 | 20 | 552 | 5 | 21 | 552 |
| 6 | 20 | 552 | 6 | 21 | 552 |
| 249 | 20 | 552 | 249 | 21 | 552 |
| 255 | 20 | 552 | 255 | 21 | 552 |
| 256 | 20 | 552 | 256 | 21 | 552 |
| 502 | 20 | 552 | 502 | 21 | 552 |
| 1 | 20 | 553 | 1 | 21 | 553 |
| 2 | 20 | 553 | 2 | 21 | 553 |
| 3 | 20 | 553 | 3 | 21 | 553 |
| 4 | 20 | 553 | 4 | 21 | 553 |
| 5 | 20 | 553 | 5 | 21 | 553 |
| 6 | 20 | 553 | 6 | 21 | 553 |
| 249 | 20 | 553 | 249 | 21 | 553 |
| 255 | 20 | 553 | 255 | 21 | 553 |
| 256 | 20 | 553 | 256 | 21 | 553 |
| 502 | 20 | 553 | 502 | 21 | 553 |
| 1 | 20 | 554 | 1 | 21 | 554 |
| 2 | 20 | 554 | 2 | 21 | 554 |
| 3 | 20 | 554 | 3 | 21 | 554 |
| 4 | 20 | 554 | 4 | 21 | 554 |
| 5 | 20 | 554 | 5 | 21 | 554 |
| 6 | 20 | 554 | 6 | 21 | 554 |
| 249 | 20 | 554 | 249 | 21 | 554 |
| 255 | 20 | 554 | 255 | 21 | 554 |
| 256 | 20 | 554 | 256 | 21 | 554 |
| 502 | 20 | 554 | 502 | 21 | 554 |
| 1 | 20 | 555 | 1 | 21 | 555 |
| 2 | 20 | 555 | 2 | 21 | 555 |
| 3 | 20 | 555 | 3 | 21 | 555 |
| 4 | 20 | 555 | 4 | 21 | 555 |
| 5 | 20 | 555 | 5 | 21 | 555 |
| 6 | 20 | 555 | 6 | 21 | 555 |
| 249 | 20 | 555 | 249 | 21 | 555 |
| 255 | 20 | 555 | 255 | 21 | 555 |
| 256 | 20 | 555 | 256 | 21 | 555 |
| 502 | 20 | 555 | 502 | 21 | 555 |
| 1 | 20 | 556 | 1 | 21 | 556 |
| 2 | 20 | 556 | 2 | 21 | 556 |
| 3 | 20 | 556 | 3 | 21 | 556 |
| 4 | 20 | 556 | 4 | 21 | 556 |
| 5 | 20 | 556 | 5 | 21 | 556 |
| 6 | 20 | 556 | 6 | 21 | 556 |
| 249 | 20 | 556 | 249 | 21 | 556 |
| 255 | 20 | 556 | 255 | 21 | 556 |
| 256 | 20 | 556 | 256 | 21 | 556 |
| 502 | 20 | 556 | 502 | 21 | 556 |
| 1 | 20 | 557 | 1 | 21 | 557 |
| 2 | 20 | 557 | 2 | 21 | 557 |
| 3 | 20 | 557 | 3 | 21 | 557 |
| 4 | 20 | 557 | 4 | 21 | 557 |
| 5 | 20 | 557 | 5 | 21 | 557 |
| 6 | 20 | 557 | 6 | 21 | 557 |
| 249 | 20 | 557 | 249 | 21 | 557 |
| 255 | 20 | 557 | 255 | 21 | 557 |
| 256 | 20 | 557 | 256 | 21 | 557 |
| 502 | 20 | 557 | 502 | 21 | 557 |
| 1 | 20 | 558 | 1 | 21 | 558 |
| 2 | 20 | 558 | 2 | 21 | 558 |
| 3 | 20 | 558 | 3 | 21 | 558 |
| 4 | 20 | 558 | 4 | 21 | 558 |
| 5 | 20 | 558 | 5 | 21 | 558 |
| 6 | 20 | 558 | 6 | 21 | 558 |
| 249 | 20 | 558 | 249 | 21 | 558 |
| 255 | 20 | 558 | 255 | 21 | 558 |
| 256 | 20 | 558 | 256 | 21 | 558 |
| 502 | 20 | 558 | 502 | 21 | 558 |
| RelB-linker | | | RelB-linker | | |
| (SEQ ID NO: 22)-RelA | | | (SEQ ID NO: 23)-RelA | | |
| 1 | 22 | 8 | 1 | 23 | 8 |
| 2 | 22 | 8 | 2 | 23 | 8 |
| 3 | 22 | 8 | 3 | 23 | 8 |
| 4 | 22 | 8 | 4 | 23 | 8 |
| 5 | 22 | 8 | 5 | 23 | 8 |
| 6 | 22 | 8 | 6 | 23 | 8 |
| 249 | 22 | 8 | 249 | 23 | 8 |
| 255 | 22 | 8 | 255 | 23 | 8 |
| 256 | 22 | 8 | 256 | 23 | 8 |
| 502 | 22 | 8 | 502 | 23 | 8 |
| 1 | 22 | 9 | 1 | 23 | 9 |
| 2 | 22 | 9 | 2 | 23 | 9 |
| 3 | 22 | 9 | 3 | 23 | 9 |
| 4 | 22 | 9 | 4 | 23 | 9 |
| 5 | 22 | 9 | 5 | 23 | 9 |
| 6 | 22 | 9 | 6 | 23 | 9 |
| 249 | 22 | 9 | 249 | 23 | 9 |
| 255 | 22 | 9 | 255 | 23 | 9 |
| 256 | 22 | 9 | 256 | 23 | 9 |
| 502 | 22 | 9 | 502 | 23 | 9 |
| 1 | 22 | 10 | 1 | 23 | 10 |
| 2 | 22 | 10 | 2 | 23 | 10 |
| 3 | 22 | 10 | 3 | 23 | 10 |
| 4 | 22 | 10 | 4 | 23 | 10 |
| 5 | 22 | 10 | 5 | 23 | 10 |
| 6 | 22 | 10 | 6 | 23 | 10 |
| 249 | 22 | 10 | 249 | 23 | 10 |
| 255 | 22 | 10 | 255 | 23 | 10 |
| 256 | 22 | 10 | 256 | 23 | 10 |
| 502 | 22 | 10 | 502 | 23 | 10 |
| 1 | 22 | 11 | 1 | 23 | 11 |
| 2 | 22 | 11 | 2 | 23 | 11 |
| 3 | 22 | 11 | 3 | 23 | 11 |
| 4 | 22 | 11 | 4 | 23 | 11 |
| 5 | 22 | 11 | 5 | 23 | 11 |
| 6 | 22 | 11 | 6 | 23 | 11 |
| 249 | 22 | 11 | 249 | 23 | 11 |
| 255 | 22 | 11 | 255 | 23 | 11 |
| 256 | 22 | 11 | 256 | 23 | 11 |
| 502 | 22 | 11 | 502 | 23 | 11 |
| 1 | 22 | 12 | 1 | 23 | 12 |
| 2 | 22 | 12 | 2 | 23 | 12 |
| 3 | 22 | 12 | 3 | 23 | 12 |
| 4 | 22 | 12 | 4 | 23 | 12 |
| 5 | 22 | 12 | 5 | 23 | 12 |
| 6 | 22 | 12 | 6 | 23 | 12 |
| 249 | 22 | 12 | 249 | 23 | 12 |
| 255 | 22 | 12 | 255 | 23 | 12 |
| 256 | 22 | 12 | 256 | 23 | 12 |
| 502 | 22 | 12 | 502 | 23 | 12 |
| 1 | 22 | 13 | 1 | 23 | 13 |
| 2 | 22 | 13 | 2 | 23 | 13 |
| 3 | 22 | 13 | 3 | 23 | 13 |
| 4 | 22 | 13 | 4 | 23 | 13 |
| 5 | 22 | 13 | 5 | 23 | 13 |
| 6 | 22 | 13 | 6 | 23 | 13 |
| 249 | 22 | 13 | 249 | 23 | 13 |
| 255 | 22 | 13 | 255 | 23 | 13 |
| 256 | 22 | 13 | 256 | 23 | 13 |
| 502 | 22 | 13 | 502 | 23 | 13 |
| 1 | 22 | 14 | 1 | 23 | 14 |
| 2 | 22 | 14 | 2 | 23 | 14 |
| 3 | 22 | 14 | 3 | 23 | 14 |
| 4 | 22 | 14 | 4 | 23 | 14 |
| 5 | 22 | 14 | 5 | 23 | 14 |
| 6 | 22 | 14 | 6 | 23 | 14 |
| 249 | 22 | 14 | 249 | 23 | 14 |
| 255 | 22 | 14 | 255 | 23 | 14 |
| 256 | 22 | 14 | 256 | 23 | 14 |
| 502 | 22 | 14 | 502 | 23 | 14 |
| 1 | 22 | 15 | 1 | 23 | 15 |
| 2 | 22 | 15 | 2 | 23 | 15 |
| 3 | 22 | 15 | 3 | 23 | 15 |
| 4 | 22 | 15 | 4 | 23 | 15 |
| 5 | 22 | 15 | 5 | 23 | 15 |
| 6 | 22 | 15 | 6 | 23 | 15 |
| 249 | 22 | 15 | 249 | 23 | 15 |
| 255 | 22 | 15 | 255 | 23 | 15 |
| 256 | 22 | 15 | 256 | 23 | 15 |
| 502 | 22 | 15 | 502 | 23 | 15 |
| 1 | 22 | 257 | 1 | 23 | 257 |
| 2 | 22 | 257 | 2 | 23 | 257 |
| 3 | 22 | 257 | 3 | 23 | 257 |
| 4 | 22 | 257 | 4 | 23 | 257 |
| 5 | 22 | 257 | 5 | 23 | 257 |
| 6 | 22 | 257 | 6 | 23 | 257 |
| 249 | 22 | 257 | 249 | 23 | 257 |
| 255 | 22 | 257 | 255 | 23 | 257 |
| 256 | 22 | 257 | 256 | 23 | 257 |
| 502 | 22 | 257 | 502 | 23 | 257 |
| 1 | 22 | 259 | 1 | 23 | 259 |
| 2 | 22 | 259 | 2 | 23 | 259 |
| 3 | 22 | 259 | 3 | 23 | 259 |
| 4 | 22 | 259 | 4 | 23 | 259 |
| 5 | 22 | 259 | 5 | 23 | 259 |
| 6 | 22 | 259 | 6 | 23 | 259 |
| 249 | 22 | 259 | 249 | 23 | 259 |
| 255 | 22 | 259 | 255 | 23 | 259 |
| 256 | 22 | 259 | 256 | 23 | 259 |
| 502 | 22 | 259 | 502 | 23 | 259 |
| 1 | 22 | 260 | 1 | 23 | 260 |
| 2 | 22 | 260 | 2 | 23 | 260 |
| 3 | 22 | 260 | 3 | 23 | 260 |
| 4 | 22 | 260 | 4 | 23 | 260 |
| 5 | 22 | 260 | 5 | 23 | 260 |
| 6 | 22 | 260 | 6 | 23 | 260 |
| 249 | 22 | 260 | 249 | 23 | 260 |
| 255 | 22 | 260 | 255 | 23 | 260 |
| 256 | 22 | 260 | 256 | 23 | 260 |
| 502 | 22 | 260 | 502 | 23 | 260 |
| 1 | 22 | 261 | 1 | 23 | 261 |
| 2 | 22 | 261 | 2 | 23 | 261 |
| 3 | 22 | 261 | 3 | 23 | 261 |
| 4 | 22 | 261 | 4 | 23 | 261 |
| 5 | 22 | 261 | 5 | 23 | 261 |
| 6 | 22 | 261 | 6 | 23 | 261 |
| 249 | 22 | 261 | 249 | 23 | 261 |
| 255 | 22 | 261 | 255 | 23 | 261 |
| 256 | 22 | 261 | 256 | 23 | 261 |
| 502 | 22 | 261 | 502 | 23 | 261 |
| 1 | 22 | 503 | 1 | 23 | 503 |
| 2 | 22 | 503 | 2 | 23 | 503 |
| 3 | 22 | 503 | 3 | 23 | 503 |
| 4 | 22 | 503 | 4 | 23 | 503 |
| 5 | 22 | 503 | 5 | 23 | 503 |
| 6 | 22 | 503 | 6 | 23 | 503 |
| 249 | 22 | 503 | 249 | 23 | 503 |
| 255 | 22 | 503 | 255 | 23 | 503 |
| 256 | 22 | 503 | 256 | 23 | 503 |
| 502 | 22 | 503 | 502 | 23 | 503 |
| 1 | 22 | 504 | 1 | 23 | 504 |
| 2 | 22 | 504 | 2 | 23 | 504 |
| 3 | 22 | 504 | 3 | 23 | 504 |
| 4 | 22 | 504 | 4 | 23 | 504 |
| 5 | 22 | 504 | 5 | 23 | 504 |
| 6 | 22 | 504 | 6 | 23 | 504 |
| 249 | 22 | 504 | 249 | 23 | 504 |
| 255 | 22 | 504 | 255 | 23 | 504 |
| 256 | 22 | 504 | 256 | 23 | 504 |
| 502 | 22 | 504 | 502 | 23 | 504 |
| 1 | 22 | 507 | 1 | 23 | 507 |
| 2 | 22 | 507 | 2 | 23 | 507 |
| 3 | 22 | 507 | 3 | 23 | 507 |
| 4 | 22 | 507 | 4 | 23 | 507 |
| 5 | 22 | 507 | 5 | 23 | 507 |
| 6 | 22 | 507 | 6 | 23 | 507 |
| 249 | 22 | 507 | 249 | 23 | 507 |
| 255 | 22 | 507 | 255 | 23 | 507 |
| 256 | 22 | 507 | 256 | 23 | 507 |
| 502 | 22 | 507 | 502 | 23 | 507 |
| 1 | 22 | 550 | 1 | 23 | 550 |
| 2 | 22 | 550 | 2 | 23 | 550 |
| 3 | 22 | 550 | 3 | 23 | 550 |
| 4 | 22 | 550 | 4 | 23 | 550 |
| 5 | 22 | 550 | 5 | 23 | 550 |
| 6 | 22 | 550 | 6 | 23 | 550 |
| 249 | 22 | 550 | 249 | 23 | 550 |
| 255 | 22 | 550 | 255 | 23 | 550 |
| 256 | 22 | 550 | 256 | 23 | 550 |
| 502 | 22 | 550 | 502 | 23 | 550 |
| 1 | 22 | 551 | 1 | 23 | 551 |
| 2 | 22 | 551 | 2 | 23 | 551 |
| 3 | 22 | 551 | 3 | 23 | 551 |
| 4 | 22 | 551 | 4 | 23 | 551 |
| 5 | 22 | 551 | 5 | 23 | 551 |
| 6 | 22 | 551 | 6 | 23 | 551 |
| 249 | 22 | 551 | 249 | 23 | 551 |
| 255 | 22 | 551 | 255 | 23 | 551 |
| 256 | 22 | 551 | 256 | 23 | 551 |
| 502 | 22 | 551 | 502 | 23 | 551 |
| 1 | 22 | 552 | 1 | 23 | 552 |
| 2 | 22 | 552 | 2 | 23 | 552 |
| 3 | 22 | 552 | 3 | 23 | 552 |
| 4 | 22 | 552 | 4 | 23 | 552 |
| 5 | 22 | 552 | 5 | 23 | 552 |
| 6 | 22 | 552 | 6 | 23 | 552 |
| 249 | 22 | 552 | 249 | 23 | 552 |
| 255 | 22 | 552 | 255 | 23 | 552 |
| 256 | 22 | 552 | 256 | 23 | 552 |
| 502 | 22 | 552 | 502 | 23 | 552 |
| 1 | 22 | 553 | 1 | 23 | 553 |
| 2 | 22 | 553 | 2 | 23 | 553 |
| 3 | 22 | 553 | 3 | 23 | 553 |
| 4 | 22 | 553 | 4 | 23 | 553 |
| 5 | 22 | 553 | 5 | 23 | 553 |
| 6 | 22 | 553 | 6 | 23 | 553 |
| 249 | 22 | 553 | 249 | 23 | 553 |
| 255 | 22 | 553 | 255 | 23 | 553 |
| 256 | 22 | 553 | 256 | 23 | 553 |
| 502 | 22 | 553 | 502 | 23 | 553 |
| 1 | 22 | 554 | 1 | 23 | 554 |
| 2 | 22 | 554 | 2 | 23 | 554 |
| 3 | 22 | 554 | 3 | 23 | 554 |
| 4 | 22 | 554 | 4 | 23 | 554 |
| 5 | 22 | 554 | 5 | 23 | 554 |
| 6 | 22 | 554 | 6 | 23 | 554 |
| 249 | 22 | 554 | 249 | 23 | 554 |
| 255 | 22 | 554 | 255 | 23 | 554 |
| 256 | 22 | 554 | 256 | 23 | 554 |
| 502 | 22 | 554 | 502 | 23 | 554 |
| 1 | 22 | 555 | 1 | 23 | 555 |
| 2 | 22 | 555 | 2 | 23 | 555 |
| 3 | 22 | 555 | 3 | 23 | 555 |
| 4 | 22 | 555 | 4 | 23 | 555 |
| 5 | 22 | 555 | 5 | 23 | 555 |
| 6 | 22 | 555 | 6 | 23 | 555 |
| 249 | 22 | 555 | 249 | 23 | 555 |
| 255 | 22 | 555 | 255 | 23 | 555 |
| 256 | 22 | 555 | 256 | 23 | 555 |
| 502 | 22 | 555 | 502 | 23 | 555 |
| 1 | 22 | 556 | 1 | 23 | 556 |
| 2 | 22 | 556 | 2 | 23 | 556 |
| 3 | 22 | 556 | 3 | 23 | 556 |
| 4 | 22 | 556 | 4 | 23 | 556 |
| 5 | 22 | 556 | 5 | 23 | 556 |
| 6 | 22 | 556 | 6 | 23 | 556 |
| 249 | 22 | 556 | 249 | 23 | 556 |
| 255 | 22 | 556 | 255 | 23 | 556 |
| 256 | 22 | 556 | 256 | 23 | 556 |
| 502 | 22 | 556 | 502 | 23 | 556 |
| 1 | 22 | 557 | 1 | 23 | 557 |
| 2 | 22 | 557 | 2 | 23 | 557 |
| 3 | 22 | 557 | 3 | 23 | 557 |
| 4 | 22 | 557 | 4 | 23 | 557 |
| 5 | 22 | 557 | 5 | 23 | 557 |
| 6 | 22 | 557 | 6 | 23 | 557 |
| 249 | 22 | 557 | 249 | 23 | 557 |
| 255 | 22 | 557 | 255 | 23 | 557 |
| 256 | 22 | 557 | 256 | 23 | 557 |
| 502 | 22 | 557 | 502 | 23 | 557 |
| 1 | 22 | 558 | 1 | 23 | 558 |
| 2 | 22 | 558 | 2 | 23 | 558 |
| 3 | 22 | 558 | 3 | 23 | 558 |
| 4 | 22 | 558 | 4 | 23 | 558 |
| 5 | 22 | 558 | 5 | 23 | 558 |
| 6 | 22 | 558 | 6 | 23 | 558 |
| 249 | 22 | 558 | 249 | 23 | 558 |
| 255 | 22 | 558 | 255 | 23 | 558 |
| 256 | 22 | 558 | 256 | 23 | 558 |
| 502 | 22 | 558 | 502 | 23 | 558 |
| RelB-linker | | | RelB-linker | | |
| (SEQ ID NO: 24)-RelA | | | (SEQ ID NO: 26)-RelA | | |
| 1 | 24 | 8 | 1 | 26 | 8 |
| 2 | 24 | 8 | 2 | 26 | 8 |
| 3 | 24 | 8 | 3 | 26 | 8 |
| 4 | 24 | 8 | 4 | 26 | 8 |
| 5 | 24 | 8 | 5 | 26 | 8 |
| 6 | 24 | 8 | 6 | 26 | 8 |
| 249 | 24 | 8 | 249 | 26 | 8 |
| 255 | 24 | 8 | 255 | 26 | 8 |
| 256 | 24 | 8 | 256 | 26 | 8 |
| 502 | 24 | 8 | 502 | 26 | 8 |
| 1 | 24 | 9 | 1 | 26 | 9 |
| 2 | 24 | 9 | 2 | 26 | 9 |
| 3 | 24 | 9 | 3 | 26 | 9 |
| 4 | 24 | 9 | 4 | 26 | 9 |
| 5 | 24 | 9 | 5 | 26 | 9 |
| 6 | 24 | 9 | 6 | 26 | 9 |
| 249 | 24 | 9 | 249 | 26 | 9 |
| 255 | 24 | 9 | 255 | 26 | 9 |
| 256 | 24 | 9 | 256 | 26 | 9 |
| 502 | 24 | 9 | 502 | 26 | 9 |
| 1 | 24 | 10 | 1 | 26 | 10 |
| 2 | 24 | 10 | 2 | 26 | 10 |
| 3 | 24 | 10 | 3 | 26 | 10 |
| 4 | 24 | 10 | 4 | 26 | 10 |
| 5 | 24 | 10 | 5 | 26 | 10 |
| 6 | 24 | 10 | 6 | 26 | 10 |
| 249 | 24 | 10 | 249 | 26 | 10 |
| 255 | 24 | 10 | 255 | 26 | 10 |
| 256 | 24 | 10 | 256 | 26 | 10 |
| 502 | 24 | 10 | 502 | 26 | 10 |
| 1 | 24 | 11 | 1 | 26 | 11 |
| 2 | 24 | 11 | 2 | 26 | 11 |
| 3 | 24 | 11 | 3 | 26 | 11 |
| 4 | 24 | 11 | 4 | 26 | 11 |
| 5 | 24 | 11 | 5 | 26 | 11 |
| 6 | 24 | 11 | 6 | 26 | 11 |
| 249 | 24 | 11 | 249 | 26 | 11 |
| 255 | 24 | 11 | 255 | 26 | 11 |
| 256 | 24 | 11 | 256 | 26 | 11 |
| 502 | 24 | 11 | 502 | 26 | 11 |
| 1 | 24 | 12 | 1 | 26 | 12 |
| 2 | 24 | 12 | 2 | 26 | 12 |
| 3 | 24 | 12 | 3 | 26 | 12 |
| 4 | 24 | 12 | 4 | 26 | 12 |
| 5 | 24 | 12 | 5 | 26 | 12 |
| 6 | 24 | 12 | 6 | 26 | 12 |
| 249 | 24 | 12 | 249 | 26 | 12 |
| 255 | 24 | 12 | 255 | 26 | 12 |
| 256 | 24 | 12 | 256 | 26 | 12 |
| 502 | 24 | 12 | 502 | 26 | 12 |
| 1 | 24 | 13 | 1 | 26 | 13 |
| 2 | 24 | 13 | 2 | 26 | 13 |
| 3 | 24 | 13 | 3 | 26 | 13 |
| 4 | 24 | 13 | 4 | 26 | 13 |
| 5 | 24 | 13 | 5 | 26 | 13 |
| 6 | 24 | 13 | 6 | 26 | 13 |
| 249 | 24 | 13 | 249 | 26 | 13 |
| 255 | 24 | 13 | 255 | 26 | 13 |
| 256 | 24 | 13 | 256 | 26 | 13 |
| 502 | 24 | 13 | 502 | 26 | 13 |
| 1 | 24 | 14 | 1 | 26 | 14 |
| 2 | 24 | 14 | 2 | 26 | 14 |
| 3 | 24 | 14 | 3 | 26 | 14 |
| 4 | 24 | 14 | 4 | 26 | 14 |
| 5 | 24 | 14 | 5 | 26 | 14 |
| 6 | 24 | 14 | 6 | 26 | 14 |
| 249 | 24 | 14 | 249 | 26 | 14 |
| 255 | 24 | 14 | 255 | 26 | 14 |
| 256 | 24 | 14 | 256 | 26 | 14 |
| 502 | 24 | 14 | 502 | 26 | 14 |
| 1 | 24 | 15 | 1 | 26 | 15 |
| 2 | 24 | 15 | 2 | 26 | 15 |
| 3 | 24 | 15 | 3 | 26 | 15 |
| 4 | 24 | 15 | 4 | 26 | 15 |
| 5 | 24 | 15 | 5 | 26 | 15 |
| 6 | 24 | 15 | 6 | 26 | 15 |
| 249 | 24 | 15 | 249 | 26 | 15 |
| 255 | 24 | 15 | 255 | 26 | 15 |
| 256 | 24 | 15 | 256 | 26 | 15 |
| 502 | 24 | 15 | 502 | 26 | 15 |
| 1 | 24 | 257 | 1 | 26 | 257 |
| 2 | 24 | 257 | 2 | 26 | 257 |
| 3 | 24 | 257 | 3 | 26 | 257 |
| 4 | 24 | 257 | 4 | 26 | 257 |
| 5 | 24 | 257 | 5 | 26 | 257 |
| 6 | 24 | 257 | 6 | 26 | 257 |
| 249 | 24 | 257 | 249 | 26 | 257 |
| 255 | 24 | 257 | 255 | 26 | 257 |
| 256 | 24 | 257 | 256 | 26 | 257 |
| 502 | 24 | 257 | 502 | 26 | 257 |
| 1 | 24 | 259 | 1 | 26 | 259 |
| 2 | 24 | 259 | 2 | 26 | 259 |
| 3 | 24 | 259 | 3 | 26 | 259 |
| 4 | 24 | 259 | 4 | 26 | 259 |
| 5 | 24 | 259 | 5 | 26 | 259 |
| 6 | 24 | 259 | 6 | 26 | 259 |
| 249 | 24 | 259 | 249 | 26 | 259 |
| 255 | 24 | 259 | 255 | 26 | 259 |
| 256 | 24 | 259 | 256 | 26 | 259 |
| 502 | 24 | 259 | 502 | 26 | 259 |
| 1 | 24 | 260 | 1 | 26 | 260 |
| 2 | 24 | 260 | 2 | 26 | 260 |
| 3 | 24 | 260 | 3 | 26 | 260 |
| 4 | 24 | 260 | 4 | 26 | 260 |
| 5 | 24 | 260 | 5 | 26 | 260 |
| 6 | 24 | 260 | 6 | 26 | 260 |
| 249 | 24 | 260 | 249 | 26 | 260 |
| 255 | 24 | 260 | 255 | 26 | 260 |
| 256 | 24 | 260 | 256 | 26 | 260 |
| 502 | 24 | 260 | 502 | 26 | 260 |
| 1 | 24 | 261 | 1 | 26 | 261 |
| 2 | 24 | 261 | 2 | 26 | 261 |
| 3 | 24 | 261 | 3 | 26 | 261 |
| 4 | 24 | 261 | 4 | 26 | 261 |
| 5 | 24 | 26 | 5 | 26 | 261 |
| 6 | 24 | 261 | 6 | 26 | 261 |
| 249 | 24 | 261 | 249 | 26 | 261 |
| 255 | 24 | 261 | 255 | 26 | 261 |
| 256 | 24 | 261 | 256 | 26 | 261 |
| 502 | 24 | 261 | 502 | 26 | 261 |
| 1 | 24 | 503 | 1 | 26 | 503 |
| 2 | 24 | 503 | 2 | 26 | 503 |
| 3 | 24 | 503 | 3 | 26 | 503 |
| 4 | 24 | 503 | 4 | 26 | 503 |
| 5 | 24 | 503 | 5 | 26 | 503 |
| 6 | 24 | 503 | 6 | 26 | 503 |
| 249 | 24 | 503 | 249 | 26 | 503 |
| 255 | 24 | 503 | 255 | 26 | 503 |
| 256 | 24 | 503 | 256 | 26 | 503 |
| 502 | 24 | 503 | 502 | 26 | 503 |
| 1 | 24 | 504 | 1 | 26 | 504 |
| 2 | 24 | 504 | 2 | 26 | 504 |
| 3 | 24 | 504 | 3 | 26 | 504 |
| 4 | 24 | 504 | 4 | 26 | 504 |
| 5 | 24 | 504 | 5 | 26 | 504 |
| 6 | 24 | 504 | 6 | 26 | 504 |
| 249 | 24 | 504 | 249 | 26 | 504 |
| 255 | 24 | 504 | 255 | 26 | 504 |
| 256 | 24 | 504 | 256 | 26 | 504 |
| 502 | 24 | 504 | 502 | 26 | 504 |
| 1 | 24 | 507 | 1 | 26 | 507 |
| 2 | 24 | 507 | 2 | 26 | 507 |
| 3 | 24 | 507 | 3 | 26 | 507 |
| 4 | 24 | 507 | 4 | 26 | 507 |
| 5 | 24 | 507 | 5 | 26 | 507 |
| 6 | 24 | 507 | 6 | 26 | 507 |
| 249 | 24 | 507 | 249 | 26 | 507 |
| 255 | 24 | 507 | 255 | 26 | 507 |
| 256 | 24 | 507 | 256 | 26 | 507 |
| 502 | 24 | 507 | 502 | 26 | 507 |
| 1 | 24 | 550 | 1 | 26 | 550 |
| 2 | 24 | 550 | 2 | 26 | 550 |
| 3 | 24 | 550 | 3 | 26 | 550 |
| 4 | 24 | 550 | 4 | 26 | 550 |
| 5 | 24 | 550 | 5 | 26 | 550 |
| 6 | 24 | 550 | 6 | 26 | 550 |
| 249 | 24 | 550 | 249 | 26 | 550 |
| 255 | 24 | 550 | 255 | 26 | 550 |
| 256 | 24 | 550 | 256 | 26 | 550 |
| 502 | 24 | 550 | 502 | 26 | 550 |
| 1 | 24 | 551 | 1 | 26 | 551 |
| 2 | 24 | 551 | 2 | 26 | 551 |
| 3 | 24 | 551 | 3 | 26 | 551 |
| 4 | 24 | 551 | 4 | 26 | 551 |
| 5 | 24 | 551 | 5 | 26 | 551 |
| 6 | 24 | 551 | 6 | 26 | 551 |
| 249 | 24 | 551 | 249 | 26 | 551 |
| 255 | 24 | 551 | 255 | 26 | 551 |
| 256 | 24 | 551 | 256 | 26 | 551 |
| 502 | 24 | 551 | 502 | 26 | 551 |
| 1 | 24 | 552 | 1 | 26 | 552 |
| 2 | 24 | 552 | 2 | 26 | 552 |
| 3 | 24 | 552 | 3 | 26 | 552 |
| 4 | 24 | 552 | 4 | 26 | 552 |
| 5 | 24 | 552 | 5 | 26 | 552 |
| 6 | 24 | 552 | 6 | 26 | 552 |
| 249 | 24 | 552 | 249 | 26 | 552 |
| 255 | 24 | 552 | 255 | 26 | 552 |
| 256 | 24 | 552 | 256 | 26 | 552 |
| 502 | 24 | 552 | 502 | 26 | 552 |
| 1 | 24 | 553 | 1 | 26 | 553 |
| 2 | 24 | 553 | 2 | 26 | 553 |
| 3 | 24 | 553 | 3 | 26 | 553 |
| 4 | 24 | 553 | 4 | 26 | 553 |
| 5 | 24 | 553 | 5 | 26 | 553 |
| 6 | 24 | 553 | 6 | 26 | 553 |
| 249 | 24 | 553 | 249 | 26 | 553 |
| 255 | 24 | 553 | 255 | 26 | 553 |
| 256 | 24 | 553 | 256 | 26 | 553 |
| 502 | 24 | 553 | 502 | 26 | 553 |
| 1 | 24 | 554 | 1 | 26 | 554 |
| 2 | 24 | 554 | 2 | 26 | 554 |
| 3 | 24 | 554 | 3 | 26 | 554 |
| 4 | 24 | 554 | 4 | 26 | 554 |
| 5 | 24 | 554 | 5 | 26 | 554 |
| 6 | 24 | 554 | 6 | 26 | 554 |
| 249 | 24 | 554 | 249 | 26 | 554 |
| 255 | 24 | 554 | 255 | 26 | 554 |
| 256 | 24 | 554 | 256 | 26 | 554 |
| 502 | 24 | 554 | 502 | 26 | 554 |
| 1 | 24 | 555 | 1 | 26 | 555 |
| 2 | 24 | 555 | 2 | 26 | 555 |
| 3 | 24 | 555 | 3 | 26 | 555 |
| 4 | 24 | 555 | 4 | 26 | 555 |
| 5 | 24 | 555 | 5 | 26 | 555 |
| 6 | 24 | 555 | 6 | 26 | 555 |
| 249 | 24 | 555 | 249 | 26 | 555 |
| 255 | 24 | 555 | 255 | 26 | 555 |
| 256 | 24 | 555 | 256 | 26 | 555 |
| 502 | 24 | 555 | 502 | 26 | 555 |
| 1 | 24 | 556 | 1 | 26 | 556 |
| 2 | 24 | 556 | 2 | 26 | 556 |
| 3 | 24 | 556 | 3 | 26 | 556 |
| 4 | 24 | 556 | 4 | 26 | 556 |
| 5 | 24 | 556 | 5 | 26 | 556 |
| 6 | 24 | 556 | 6 | 26 | 556 |
| 249 | 24 | 556 | 249 | 26 | 556 |
| 255 | 24 | 556 | 255 | 26 | 556 |
| 256 | 24 | 556 | 256 | 26 | 556 |
| 502 | 24 | 556 | 502 | 26 | 556 |
| 1 | 24 | 557 | 1 | 26 | 557 |
| 2 | 24 | 557 | 2 | 26 | 557 |
| 3 | 24 | 557 | 3 | 26 | 557 |
| 4 | 24 | 557 | 4 | 26 | 557 |
| 5 | 24 | 557 | 5 | 26 | 557 |
| 6 | 24 | 557 | 6 | 26 | 557 |
| 249 | 24 | 557 | 249 | 26 | 557 |
| 255 | 24 | 557 | 255 | 26 | 557 |
| 256 | 24 | 557 | 256 | 26 | 557 |
| 502 | 24 | 557 | 502 | 26 | 557 |
| 1 | 24 | 558 | 1 | 26 | 558 |
| 2 | 24 | 558 | 2 | 26 | 558 |
| 3 | 24 | 558 | 3 | 26 | 558 |
| 4 | 24 | 558 | 4 | 26 | 558 |
| 5 | 24 | 558 | 5 | 26 | 558 |
| 6 | 24 | 558 | 6 | 26 | 558 |
| 249 | 24 | 558 | 249 | 26 | 558 |
| 255 | 24 | 558 | 255 | 26 | 558 |
| 256 | 24 | 558 | 256 | 26 | 558 |
| 502 | 24 | 558 | 502 | 26 | 558 |
| RelB-linker | | | RelB-linker | | |
| (SEQ ID NO: 27)-RelA | | | (SEQ ID NO: 272)-RelA | | |
| 1 | 27 | 8 | 1 | 272 | 8 |
| 2 | 27 | 8 | 2 | 272 | 8 |
| 3 | 27 | 8 | 3 | 272 | 8 |
| 4 | 27 | 8 | 4 | 272 | 8 |
| 5 | 27 | 8 | 5 | 272 | 8 |
| 6 | 27 | 8 | 6 | 272 | 8 |
| 249 | 27 | 8 | 249 | 272 | 8 |
| 255 | 27 | 8 | 255 | 272 | 8 |
| 256 | 27 | 8 | 256 | 272 | 8 |
| 502 | 27 | 8 | 502 | 272 | 8 |
| 1 | 27 | 9 | 1 | 272 | 9 |
| 2 | 27 | 9 | 2 | 272 | 9 |
| 3 | 27 | 9 | 3 | 272 | 9 |
| 4 | 27 | 9 | 4 | 272 | 9 |
| 5 | 27 | 9 | 5 | 272 | 9 |
| 6 | 27 | 9 | 6 | 272 | 9 |
| 249 | 27 | 9 | 249 | 272 | 9 |
| 255 | 27 | 9 | 255 | 272 | 9 |
| 256 | 27 | 9 | 256 | 272 | 9 |
| 502 | 27 | 9 | 502 | 272 | 9 |
| 1 | 27 | 10 | 1 | 272 | 10 |
| 2 | 27 | 10 | 2 | 272 | 10 |
| 3 | 27 | 10 | 3 | 272 | 10 |
| 4 | 27 | 10 | 4 | 272 | 10 |
| 5 | 27 | 10 | 5 | 272 | 10 |
| 6 | 27 | 10 | 6 | 272 | 10 |
| 249 | 27 | 10 | 249 | 272 | 10 |
| 255 | 27 | 10 | 255 | 272 | 10 |
| 256 | 27 | 10 | 256 | 272 | 10 |
| 502 | 27 | 10 | 502 | 272 | 10 |
| 1 | 27 | 11 | 1 | 272 | 11 |
| 2 | 27 | 11 | 2 | 272 | 11 |
| 3 | 27 | 11 | 3 | 272 | 11 |
| 4 | 27 | 11 | 4 | 272 | 11 |
| 5 | 27 | 11 | 5 | 272 | 11 |
| 6 | 27 | 11 | 6 | 272 | 11 |
| 249 | 27 | 11 | 249 | 272 | 11 |
| 255 | 27 | 11 | 255 | 272 | 11 |
| 256 | 27 | 11 | 256 | 272 | 11 |
| 502 | 27 | 11 | 502 | 272 | 11 |
| 1 | 27 | 12 | 1 | 272 | 12 |
| 2 | 27 | 12 | 2 | 272 | 12 |
| 3 | 27 | 12 | 3 | 272 | 12 |
| 4 | 27 | 12 | 4 | 272 | 12 |
| 5 | 27 | 12 | 5 | 272 | 12 |
| 6 | 27 | 12 | 6 | 272 | 12 |
| 249 | 27 | 12 | 249 | 272 | 12 |
| 255 | 27 | 12 | 255 | 272 | 12 |
| 256 | 27 | 12 | 256 | 272 | 12 |
| 502 | 27 | 12 | 502 | 272 | 12 |
| 1 | 27 | 13 | 1 | 272 | 13 |
| 2 | 27 | 13 | 2 | 272 | 13 |
| 3 | 27 | 13 | 3 | 272 | 13 |
| 4 | 27 | 13 | 4 | 272 | 13 |
| 5 | 27 | 13 | 5 | 272 | 13 |
| 6 | 27 | 13 | 6 | 272 | 13 |
| 249 | 27 | 13 | 249 | 272 | 13 |
| 255 | 27 | 13 | 255 | 272 | 13 |
| 256 | 27 | 13 | 256 | 272 | 13 |
| 502 | 27 | 13 | 502 | 272 | 13 |
| 1 | 27 | 14 | 1 | 272 | 14 |
| 2 | 27 | 14 | 2 | 272 | 14 |
| 3 | 27 | 14 | 3 | 272 | 14 |
| 4 | 27 | 14 | 4 | 272 | 14 |
| 5 | 27 | 14 | 5 | 272 | 14 |
| 6 | 27 | 14 | 6 | 272 | 14 |
| 249 | 27 | 14 | 249 | 272 | 14 |
| 255 | 27 | 14 | 255 | 272 | 14 |
| 256 | 27 | 14 | 256 | 272 | 14 |
| 502 | 27 | 14 | 502 | 272 | 14 |
| 1 | 27 | 15 | 1 | 272 | 15 |
| 2 | 27 | 15 | 2 | 272 | 15 |
| 3 | 27 | 15 | 3 | 272 | 15 |
| 4 | 27 | 15 | 4 | 272 | 15 |
| 5 | 27 | 15 | 5 | 272 | 15 |
| 6 | 27 | 15 | 6 | 272 | 15 |
| 249 | 27 | 15 | 249 | 272 | 15 |
| 255 | 27 | 15 | 255 | 272 | 15 |
| 256 | 27 | 15 | 256 | 272 | 15 |
| 502 | 27 | 15 | 502 | 272 | 15 |
| 1 | 27 | 257 | 1 | 272 | 257 |
| 2 | 27 | 257 | 2 | 272 | 257 |
| 3 | 27 | 257 | 3 | 272 | 257 |
| 4 | 27 | 257 | 4 | 272 | 257 |
| 5 | 27 | 257 | 5 | 272 | 257 |
| 6 | 27 | 257 | 6 | 272 | 257 |
| 249 | 27 | 257 | 249 | 272 | 257 |
| 255 | 27 | 257 | 255 | 272 | 257 |
| 256 | 27 | 257 | 256 | 272 | 257 |
| 502 | 27 | 257 | 502 | 272 | 257 |
| 1 | 27 | 259 | 1 | 272 | 259 |
| 2 | 27 | 259 | 2 | 272 | 259 |
| 3 | 27 | 259 | 3 | 272 | 259 |
| 4 | 27 | 259 | 4 | 272 | 259 |
| 5 | 27 | 259 | 5 | 272 | 259 |
| 6 | 27 | 259 | 6 | 272 | 259 |
| 249 | 27 | 259 | 249 | 272 | 259 |
| 255 | 27 | 259 | 255 | 272 | 259 |
| 256 | 27 | 259 | 256 | 272 | 259 |
| 502 | 27 | 259 | 502 | 272 | 259 |
| 1 | 27 | 260 | 1 | 272 | 260 |
| 2 | 27 | 260 | 2 | 272 | 260 |
| 3 | 27 | 260 | 3 | 272 | 260 |
| 4 | 27 | 260 | 4 | 272 | 260 |
| 5 | 27 | 260 | 5 | 272 | 260 |
| 6 | 27 | 260 | 6 | 272 | 260 |
| 249 | 27 | 260 | 249 | 272 | 260 |
| 255 | 27 | 260 | 255 | 272 | 260 |
| 256 | 27 | 260 | 256 | 272 | 260 |
| 502 | 27 | 260 | 502 | 272 | 260 |
| 1 | 27 | 261 | 1 | 272 | 261 |
| 2 | 27 | 261 | 2 | 272 | 261 |
| 3 | 27 | 261 | 3 | 272 | 261 |
| 4 | 27 | 261 | 4 | 272 | 261 |
| 5 | 27 | 261 | 5 | 272 | 261 |
| 6 | 27 | 261 | 6 | 272 | 261 |
| 249 | 27 | 261 | 249 | 272 | 261 |
| 255 | 27 | 261 | 255 | 272 | 261 |
| 256 | 27 | 261 | 256 | 272 | 261 |
| 502 | 27 | 261 | 502 | 272 | 261 |
| 1 | 27 | 503 | 1 | 272 | 503 |
| 2 | 27 | 503 | 2 | 272 | 503 |
| 3 | 27 | 503 | 3 | 272 | 503 |
| 4 | 27 | 503 | 4 | 272 | 503 |
| 5 | 27 | 503 | 5 | 272 | 503 |
| 6 | 27 | 503 | 6 | 272 | 503 |
| 249 | 27 | 503 | 249 | 272 | 503 |
| 255 | 27 | 503 | 255 | 272 | 503 |
| 256 | 27 | 503 | 256 | 272 | 503 |
| 502 | 27 | 503 | 502 | 272 | 503 |
| 1 | 27 | 504 | 1 | 272 | 504 |
| 2 | 27 | 504 | 2 | 272 | 504 |
| 3 | 27 | 504 | 3 | 272 | 504 |
| 4 | 27 | 504 | 4 | 272 | 504 |
| 5 | 27 | 504 | 5 | 272 | 504 |
| 6 | 27 | 504 | 6 | 272 | 504 |
| 249 | 27 | 504 | 249 | 272 | 504 |
| 255 | 27 | 504 | 255 | 272 | 504 |
| 256 | 27 | 504 | 256 | 272 | 504 |
| 502 | 27 | 504 | 502 | 272 | 504 |
| 1 | 27 | 507 | 1 | 272 | 507 |
| 2 | 27 | 507 | 2 | 272 | 507 |
| 3 | 27 | 507 | 3 | 272 | 507 |
| 4 | 27 | 507 | 4 | 272 | 507 |
| 5 | 27 | 507 | 5 | 272 | 507 |
| 6 | 27 | 507 | 6 | 272 | 507 |
| 249 | 27 | 507 | 249 | 272 | 507 |
| 255 | 27 | 507 | 255 | 272 | 507 |
| 256 | 27 | 507 | 256 | 272 | 507 |
| 502 | 27 | 507 | 502 | 272 | 507 |
| 1 | 27 | 550 | 1 | 272 | 550 |
| 2 | 27 | 550 | 2 | 272 | 550 |
| 3 | 27 | 550 | 3 | 272 | 550 |
| 4 | 27 | 550 | 4 | 272 | 550 |
| 5 | 27 | 550 | 5 | 272 | 550 |
| 6 | 27 | 550 | 6 | 272 | 550 |
| 249 | 27 | 550 | 249 | 272 | 550 |
| 255 | 27 | 550 | 255 | 272 | 550 |
| 256 | 27 | 550 | 256 | 272 | 550 |
| 502 | 27 | 550 | 502 | 272 | 550 |
| 1 | 27 | 551 | 1 | 272 | 551 |
| 2 | 27 | 551 | 2 | 272 | 551 |
| 3 | 27 | 551 | 3 | 272 | 551 |
| 4 | 27 | 551 | 4 | 272 | 551 |
| 5 | 27 | 551 | 5 | 272 | 551 |
| 6 | 27 | 551 | 6 | 272 | 551 |
| 249 | 27 | 551 | 249 | 272 | 551 |
| 255 | 27 | 551 | 255 | 272 | 551 |
| 256 | 27 | 551 | 256 | 272 | 551 |
| 502 | 27 | 551 | 502 | 272 | 551 |
| 1 | 27 | 552 | 1 | 272 | 552 |
| 2 | 27 | 552 | 2 | 272 | 552 |
| 3 | 27 | 552 | 3 | 272 | 552 |
| 4 | 27 | 552 | 4 | 272 | 552 |
| 5 | 27 | 552 | 5 | 272 | 552 |
| 6 | 27 | 552 | 6 | 272 | 552 |
| 249 | 27 | 552 | 249 | 272 | 552 |
| 255 | 27 | 552 | 255 | 272 | 552 |
| 256 | 27 | 552 | 256 | 272 | 552 |
| 502 | 27 | 552 | 502 | 272 | 552 |
| 1 | 27 | 553 | 1 | 272 | 553 |
| 2 | 27 | 553 | 2 | 272 | 553 |
| 3 | 27 | 553 | 3 | 272 | 553 |
| 4 | 27 | 553 | 4 | 272 | 553 |
| 5 | 27 | 553 | 5 | 272 | 553 |
| 6 | 27 | 553 | 6 | 272 | 553 |
| 249 | 27 | 553 | 249 | 272 | 553 |
| 255 | 27 | 553 | 255 | 272 | 553 |
| 256 | 27 | 553 | 256 | 272 | 553 |
| 502 | 27 | 553 | 502 | 272 | 553 |
| 1 | 27 | 554 | 1 | 272 | 554 |
| 2 | 27 | 554 | 2 | 272 | 554 |
| 3 | 27 | 554 | 3 | 272 | 554 |
| 4 | 27 | 554 | 4 | 272 | 554 |
| 5 | 27 | 554 | 5 | 272 | 554 |
| 6 | 27 | 554 | 6 | 272 | 554 |
| 249 | 27 | 554 | 249 | 272 | 554 |
| 255 | 27 | 554 | 255 | 272 | 554 |
| 256 | 27 | 554 | 256 | 272 | 554 |
| 502 | 27 | 554 | 502 | 272 | 554 |
| 1 | 27 | 555 | 1 | 272 | 555 |
| 2 | 27 | 555 | 2 | 272 | 555 |
| 3 | 27 | 555 | 3 | 272 | 555 |
| 4 | 27 | 555 | 4 | 272 | 555 |
| 5 | 27 | 555 | 5 | 272 | 555 |
| 6 | 27 | 555 | 6 | 272 | 555 |
| 249 | 27 | 555 | 249 | 272 | 555 |
| 255 | 27 | 555 | 255 | 272 | 555 |
| 256 | 27 | 555 | 256 | 272 | 555 |
| 502 | 27 | 555 | 502 | 272 | 555 |
| 1 | 27 | 556 | 1 | 272 | 556 |
| 2 | 27 | 556 | 2 | 272 | 556 |
| 3 | 27 | 556 | 3 | 272 | 556 |
| 4 | 27 | 556 | 4 | 272 | 556 |
| 5 | 27 | 556 | 5 | 272 | 556 |
| 6 | 27 | 556 | 6 | 272 | 556 |
| 249 | 27 | 556 | 249 | 272 | 556 |
| 255 | 27 | 556 | 255 | 272 | 556 |
| 256 | 27 | 556 | 256 | 272 | 556 |
| 502 | 27 | 556 | 502 | 272 | 556 |
| 1 | 27 | 557 | 1 | 272 | 557 |
| 2 | 27 | 557 | 2 | 272 | 557 |
| 3 | 27 | 557 | 3 | 272 | 557 |
| 4 | 27 | 557 | 4 | 272 | 557 |
| 5 | 27 | 557 | 5 | 272 | 557 |
| 6 | 27 | 557 | 6 | 272 | 557 |
| 249 | 27 | 557 | 249 | 272 | 557 |
| 255 | 27 | 557 | 255 | 272 | 557 |
| 256 | 27 | 557 | 256 | 272 | 557 |
| 502 | 27 | 557 | 502 | 272 | 557 |
| 1 | 27 | 558 | 1 | 272 | 558 |
| 2 | 27 | 558 | 2 | 272 | 558 |
| 3 | 27 | 558 | 3 | 272 | 558 |
| 4 | 27 | 558 | 4 | 272 | 558 |
| 5 | 27 | 558 | 5 | 272 | 558 |
| 6 | 27 | 558 | 6 | 272 | 558 |
| 249 | 27 | 558 | 249 | 272 | 558 |
| 255 | 27 | 558 | 255 | 272 | 558 |
| 256 | 27 | 558 | 256 | 272 | 558 |
| 502 | 27 | 558 | 502 | 272 | 558 |
| RelB-linker | | | RelB-linker | | |
| (SEQ ID NO: 273)-RelA | | | (SEQ ID NO: 274)-RelA | | |
| 1 | 273 | 8 | 1 | 274 | 8 |
| 2 | 273 | 8 | 2 | 274 | 8 |
| 3 | 273 | 8 | 3 | 274 | 8 |
| 4 | 273 | 8 | 4 | 274 | 8 |
| 5 | 273 | 8 | 5 | 274 | 8 |
| 6 | 273 | 8 | 6 | 274 | 8 |
| 249 | 273 | 8 | 249 | 274 | 8 |
| 255 | 273 | 8 | 255 | 274 | 8 |
| 256 | 273 | 8 | 256 | 274 | 8 |
| 502 | 273 | 8 | 502 | 274 | 8 |
| 1 | 273 | 9 | 1 | 274 | 9 |
| 2 | 273 | 9 | 2 | 274 | 9 |
| 3 | 273 | 9 | 3 | 274 | 9 |
| 4 | 273 | 9 | 4 | 274 | 9 |
| 5 | 273 | 9 | 5 | 274 | 9 |
| 6 | 273 | 9 | 6 | 274 | 9 |
| 249 | 273 | 9 | 249 | 274 | 9 |
| 255 | 273 | 9 | 255 | 274 | 9 |
| 256 | 273 | 9 | 256 | 274 | 9 |
| 502 | 273 | 9 | 502 | 274 | 9 |
| 1 | 273 | 10 | 1 | 274 | 10 |
| 2 | 273 | 10 | 2 | 274 | 10 |
| 3 | 273 | 10 | 3 | 274 | 10 |
| 4 | 273 | 10 | 4 | 274 | 10 |
| 5 | 273 | 10 | 5 | 274 | 10 |
| 6 | 273 | 10 | 6 | 274 | 10 |
| 249 | 273 | 10 | 249 | 274 | 10 |
| 255 | 273 | 10 | 255 | 274 | 10 |
| 256 | 273 | 10 | 256 | 274 | 10 |
| 502 | 273 | 10 | 502 | 274 | 10 |
| 1 | 273 | 11 | 1 | 274 | 11 |
| 2 | 273 | 11 | 2 | 274 | 11 |
| 3 | 273 | 11 | 3 | 274 | 11 |
| 4 | 273 | 11 | 4 | 274 | 11 |
| 5 | 273 | 11 | 5 | 274 | 11 |
| 6 | 273 | 11 | 6 | 274 | 11 |
| 249 | 273 | 11 | 249 | 274 | 11 |
| 255 | 273 | 11 | 255 | 274 | 11 |
| 256 | 273 | 11 | 256 | 274 | 11 |
| 502 | 273 | 11 | 502 | 274 | 11 |
| 1 | 273 | 12 | 1 | 274 | 12 |
| 2 | 273 | 12 | 2 | 274 | 12 |
| 3 | 273 | 12 | 3 | 274 | 12 |
| 4 | 273 | 12 | 4 | 274 | 12 |
| 5 | 273 | 12 | 5 | 274 | 12 |
| 6 | 273 | 12 | 6 | 274 | 12 |
| 249 | 273 | 12 | 249 | 274 | 12 |
| 255 | 273 | 12 | 255 | 274 | 12 |
| 256 | 273 | 12 | 256 | 274 | 12 |
| 502 | 273 | 12 | 502 | 274 | 12 |
| 1 | 273 | 13 | 1 | 274 | 13 |
| 2 | 273 | 13 | 2 | 274 | 13 |
| 3 | 273 | 13 | 3 | 274 | 13 |
| 4 | 273 | 13 | 4 | 274 | 13 |
| 5 | 273 | 13 | 5 | 274 | 13 |
| 6 | 273 | 13 | 6 | 274 | 13 |
| 249 | 273 | 13 | 249 | 274 | 13 |
| 255 | 273 | 13 | 255 | 274 | 13 |
| 256 | 273 | 13 | 256 | 274 | 13 |
| 502 | 273 | 13 | 502 | 274 | 13 |
| 1 | 273 | 14 | 1 | 274 | 14 |
| 2 | 273 | 14 | 2 | 274 | 14 |
| 3 | 273 | 14 | 3 | 274 | 14 |
| 4 | 273 | 14 | 4 | 274 | 14 |
| 5 | 273 | 14 | 5 | 274 | 14 |
| 6 | 273 | 14 | 6 | 274 | 14 |
| 249 | 273 | 14 | 249 | 274 | 14 |
| 255 | 273 | 14 | 255 | 274 | 14 |
| 256 | 273 | 14 | 256 | 274 | 14 |
| 502 | 273 | 14 | 502 | 274 | 14 |
| 1 | 273 | 15 | 1 | 274 | 15 |
| 2 | 273 | 15 | 2 | 274 | 15 |
| 3 | 273 | 15 | 3 | 274 | 15 |
| 4 | 273 | 15 | 4 | 274 | 15 |
| 5 | 273 | 15 | 5 | 274 | 15 |
| 6 | 273 | 15 | 6 | 274 | 15 |
| 249 | 273 | 15 | 249 | 274 | 15 |
| 255 | 273 | 15 | 255 | 274 | 15 |
| 256 | 273 | 15 | 256 | 274 | 15 |
| 502 | 273 | 15 | 502 | 274 | 15 |
| 1 | 273 | 257 | 1 | 274 | 257 |
| 2 | 273 | 257 | 2 | 274 | 257 |
| 3 | 273 | 257 | 3 | 274 | 257 |
| 4 | 273 | 257 | 4 | 274 | 257 |
| 5 | 273 | 257 | 5 | 274 | 257 |
| 6 | 273 | 257 | 6 | 274 | 257 |
| 249 | 273 | 257 | 249 | 274 | 257 |
| 255 | 273 | 257 | 255 | 274 | 257 |
| 256 | 273 | 257 | 256 | 274 | 257 |
| 502 | 273 | 257 | 502 | 274 | 257 |
| 1 | 273 | 259 | 1 | 274 | 259 |
| 2 | 273 | 259 | 2 | 274 | 259 |
| 3 | 273 | 259 | 3 | 274 | 259 |
| 4 | 273 | 259 | 4 | 274 | 259 |
| 5 | 273 | 259 | 5 | 274 | 259 |
| 6 | 273 | 259 | 6 | 274 | 259 |
| 249 | 273 | 259 | 249 | 274 | 259 |
| 255 | 273 | 259 | 255 | 274 | 259 |
| 256 | 273 | 259 | 256 | 274 | 259 |
| 502 | 273 | 259 | 502 | 274 | 259 |
| 1 | 273 | 260 | 1 | 274 | 260 |
| 2 | 273 | 260 | 2 | 274 | 260 |
| 3 | 273 | 260 | 3 | 274 | 260 |
| 4 | 273 | 260 | 4 | 274 | 260 |
| 5 | 273 | 260 | 5 | 274 | 260 |
| 6 | 273 | 260 | 6 | 274 | 260 |
| 249 | 273 | 260 | 249 | 274 | 260 |
| 255 | 273 | 260 | 255 | 274 | 260 |
| 256 | 273 | 260 | 256 | 274 | 260 |
| 502 | 273 | 260 | 502 | 274 | 260 |
| 1 | 273 | 261 | 1 | 274 | 261 |
| 2 | 273 | 261 | 2 | 274 | 261 |
| 3 | 273 | 261 | 3 | 274 | 261 |
| 4 | 273 | 261 | 4 | 274 | 261 |
| 5 | 273 | 261 | 5 | 274 | 261 |
| 6 | 273 | 261 | 6 | 274 | 261 |
| 249 | 273 | 261 | 249 | 274 | 261 |
| 255 | 273 | 261 | 255 | 274 | 261 |
| 256 | 273 | 261 | 256 | 274 | 261 |
| 502 | 273 | 261 | 502 | 274 | 261 |
| 1 | 273 | 503 | 1 | 274 | 503 |
| 2 | 273 | 503 | 2 | 274 | 503 |
| 3 | 273 | 503 | 3 | 274 | 503 |
| 4 | 273 | 503 | 4 | 274 | 503 |
| 5 | 273 | 503 | 5 | 274 | 503 |
| 6 | 273 | 503 | 6 | 274 | 503 |
| 249 | 273 | 503 | 249 | 274 | 503 |
| 255 | 273 | 503 | 255 | 274 | 503 |
| 256 | 273 | 503 | 256 | 274 | 503 |
| 502 | 273 | 503 | 502 | 274 | 503 |
| 1 | 273 | 504 | 1 | 274 | 504 |
| 2 | 273 | 504 | 2 | 274 | 504 |
| 3 | 273 | 504 | 3 | 274 | 504 |
| 4 | 273 | 504 | 4 | 274 | 504 |
| 5 | 273 | 504 | 5 | 274 | 504 |
| 6 | 273 | 504 | 6 | 274 | 504 |
| 249 | 273 | 504 | 249 | 274 | 504 |
| 255 | 273 | 504 | 255 | 274 | 504 |
| 256 | 273 | 504 | 256 | 274 | 504 |
| 502 | 273 | 504 | 502 | 274 | 504 |
| 1 | 273 | 507 | 1 | 274 | 507 |
| 2 | 273 | 507 | 2 | 274 | 507 |
| 3 | 273 | 507 | 3 | 274 | 507 |
| 4 | 273 | 507 | 4 | 274 | 507 |
| 5 | 273 | 507 | 5 | 274 | 507 |
| 6 | 273 | 507 | 6 | 274 | 507 |
| 249 | 273 | 507 | 249 | 274 | 507 |
| 255 | 273 | 507 | 255 | 274 | 507 |
| 256 | 273 | 507 | 256 | 274 | 507 |
| 502 | 273 | 507 | 502 | 274 | 507 |
| 1 | 273 | 550 | 1 | 274 | 550 |
| 2 | 273 | 550 | 2 | 274 | 550 |
| 3 | 273 | 550 | 3 | 274 | 550 |
| 4 | 273 | 550 | 4 | 274 | 550 |
| 5 | 273 | 550 | 5 | 274 | 550 |
| 6 | 273 | 550 | 6 | 274 | 550 |
| 249 | 273 | 550 | 249 | 274 | 550 |
| 255 | 273 | 550 | 255 | 274 | 550 |
| 256 | 273 | 550 | 256 | 274 | 550 |
| 502 | 273 | 550 | 502 | 274 | 550 |
| 1 | 273 | 551 | 1 | 274 | 551 |
| 2 | 273 | 551 | 2 | 274 | 551 |
| 3 | 273 | 551 | 3 | 274 | 551 |
| 4 | 273 | 551 | 4 | 274 | 551 |
| 5 | 273 | 551 | 5 | 274 | 551 |
| 6 | 273 | 551 | 6 | 274 | 551 |
| 249 | 273 | 551 | 249 | 274 | 551 |
| 255 | 273 | 551 | 255 | 274 | 551 |
| 256 | 273 | 551 | 256 | 274 | 551 |
| 502 | 273 | 551 | 502 | 274 | 551 |
| 1 | 273 | 552 | 1 | 274 | 552 |
| 2 | 273 | 552 | 2 | 274 | 552 |
| 3 | 273 | 552 | 3 | 274 | 552 |
| 4 | 273 | 552 | 4 | 274 | 552 |
| 5 | 273 | 552 | 5 | 274 | 552 |
| 6 | 273 | 552 | 6 | 274 | 552 |
| 249 | 273 | 552 | 249 | 274 | 552 |
| 255 | 273 | 552 | 255 | 274 | 552 |
| 256 | 273 | 552 | 256 | 274 | 552 |
| 502 | 273 | 552 | 502 | 274 | 552 |
| 1 | 273 | 553 | 1 | 274 | 553 |
| 2 | 273 | 553 | 2 | 274 | 553 |
| 3 | 273 | 553 | 3 | 274 | 553 |
| 4 | 273 | 553 | 4 | 274 | 553 |
| 5 | 273 | 553 | 5 | 274 | 553 |
| 6 | 273 | 553 | 6 | 274 | 553 |
| 249 | 273 | 553 | 249 | 274 | 553 |
| 255 | 273 | 553 | 255 | 274 | 553 |
| 256 | 273 | 553 | 256 | 274 | 553 |
| 502 | 273 | 553 | 502 | 274 | 553 |
| 1 | 273 | 554 | 1 | 274 | 554 |
| 2 | 273 | 554 | 2 | 274 | 554 |
| 3 | 273 | 554 | 3 | 274 | 554 |
| 4 | 273 | 554 | 4 | 274 | 554 |
| 5 | 273 | 554 | 5 | 274 | 554 |
| 6 | 273 | 554 | 6 | 274 | 554 |
| 249 | 273 | 554 | 249 | 274 | 554 |
| 255 | 273 | 554 | 255 | 274 | 554 |
| 256 | 273 | 554 | 256 | 274 | 554 |
| 502 | 273 | 554 | 502 | 274 | 554 |
| 1 | 273 | 555 | 1 | 274 | 555 |
| 2 | 273 | 555 | 2 | 274 | 555 |
| 3 | 273 | 555 | 3 | 274 | 555 |
| 4 | 273 | 555 | 4 | 274 | 555 |
| 5 | 273 | 555 | 5 | 274 | 555 |
| 6 | 273 | 555 | 6 | 274 | 555 |
| 249 | 273 | 555 | 249 | 274 | 555 |
| 255 | 273 | 555 | 255 | 274 | 555 |
| 256 | 273 | 555 | 256 | 274 | 555 |
| 502 | 273 | 555 | 502 | 274 | 555 |
| 1 | 273 | 556 | 1 | 274 | 556 |
| 2 | 273 | 556 | 2 | 274 | 556 |
| 3 | 273 | 556 | 3 | 274 | 556 |
| 4 | 273 | 556 | 4 | 274 | 556 |
| 5 | 273 | 556 | 5 | 274 | 556 |
| 6 | 273 | 556 | 6 | 274 | 556 |
| 249 | 273 | 556 | 249 | 274 | 556 |
| 255 | 273 | 556 | 255 | 274 | 556 |
| 256 | 273 | 556 | 256 | 274 | 556 |
| 502 | 273 | 556 | 502 | 274 | 556 |
| 1 | 273 | 557 | 1 | 274 | 557 |
| 2 | 273 | 557 | 2 | 274 | 557 |
| 3 | 273 | 557 | 3 | 274 | 557 |
| 4 | 273 | 557 | 4 | 274 | 557 |
| 5 | 273 | 557 | 5 | 274 | 557 |
| 6 | 273 | 557 | 6 | 274 | 557 |
| 249 | 273 | 557 | 249 | 274 | 557 |
| 255 | 273 | 557 | 255 | 274 | 557 |
| 256 | 273 | 557 | 256 | 274 | 557 |
| 502 | 273 | 557 | 502 | 274 | 557 |
| 1 | 273 | 558 | 1 | 274 | 558 |
| 2 | 273 | 558 | 2 | 274 | 558 |
| 3 | 273 | 558 | 3 | 274 | 558 |
| 4 | 273 | 558 | 4 | 274 | 558 |
| 5 | 273 | 558 | 5 | 274 | 558 |
| 6 | 273 | 558 | 6 | 274 | 558 |
| 249 | 273 | 558 | 249 | 274 | 558 |
| 255 | 273 | 558 | 255 | 274 | 558 |
| 256 | 273 | 558 | 256 | 274 | 558 |
| 502 | 273 | 558 | 502 | 274 | 558 |
| RelB-linker | | | RelB-linker | | |
| (SEQ ID NO: 275)-RelA | | | (SEQ ID NO: 276)-RelA | | |
| 1 | 275 | 8 | 1 | 276 | 8 |
| 2 | 275 | 8 | 2 | 276 | 8 |
| 3 | 275 | 8 | 3 | 276 | 8 |
| 4 | 275 | 8 | 4 | 276 | 8 |
| 5 | 275 | 8 | 5 | 276 | 8 |
| 6 | 275 | 8 | 6 | 276 | 8 |
| 249 | 275 | 8 | 249 | 276 | 8 |
| 255 | 275 | 8 | 255 | 276 | 8 |
| 256 | 275 | 8 | 256 | 276 | 8 |
| 502 | 275 | 8 | 502 | 276 | 8 |
| 1 | 275 | 9 | 1 | 276 | 9 |
| 2 | 275 | 9 | 2 | 276 | 9 |
| 3 | 275 | 9 | 3 | 276 | 9 |
| 4 | 275 | 9 | 4 | 276 | 9 |
| 5 | 275 | 9 | 5 | 276 | 9 |
| 6 | 275 | 9 | 6 | 276 | 9 |
| 249 | 275 | 9 | 249 | 276 | 9 |
| 255 | 275 | 9 | 255 | 276 | 9 |
| 256 | 275 | 9 | 256 | 276 | 9 |
| 502 | 275 | 9 | 502 | 276 | 9 |
| 1 | 275 | 10 | 1 | 276 | 10 |
| 2 | 275 | 10 | 2 | 276 | 10 |
| 3 | 275 | 10 | 3 | 276 | 10 |
| 4 | 275 | 10 | 4 | 276 | 10 |
| 5 | 275 | 10 | 5 | 276 | 10 |
| 6 | 275 | 10 | 6 | 276 | 10 |
| 249 | 275 | 10 | 249 | 276 | 10 |
| 255 | 275 | 10 | 255 | 276 | 10 |
| 256 | 275 | 10 | 256 | 276 | 10 |
| 502 | 275 | 10 | 502 | 276 | 10 |
| 1 | 275 | 11 | 1 | 276 | 11 |
| 2 | 275 | 11 | 2 | 276 | 11 |
| 3 | 275 | 11 | 3 | 276 | 11 |
| 4 | 275 | 11 | 4 | 276 | 11 |
| 5 | 275 | 11 | 5 | 276 | 11 |
| 6 | 275 | 11 | 6 | 276 | 11 |
| 249 | 275 | 11 | 249 | 276 | 11 |
| 255 | 275 | 11 | 255 | 276 | 11 |
| 256 | 275 | 11 | 256 | 276 | 11 |
| 502 | 275 | 11 | 502 | 276 | 11 |
| 1 | 275 | 12 | 1 | 276 | 12 |
| 2 | 275 | 12 | 2 | 276 | 12 |
| 3 | 275 | 12 | 3 | 276 | 12 |
| 4 | 275 | 12 | 4 | 276 | 12 |
| 5 | 275 | 12 | 5 | 276 | 12 |
| 6 | 275 | 12 | 6 | 276 | 12 |
| 249 | 275 | 12 | 249 | 276 | 12 |
| 255 | 275 | 12 | 255 | 276 | 12 |
| 256 | 275 | 12 | 256 | 276 | 12 |
| 502 | 275 | 12 | 502 | 276 | 12 |
| 1 | 275 | 13 | 1 | 276 | 13 |
| 2 | 275 | 13 | 2 | 276 | 13 |
| 3 | 275 | 13 | 3 | 276 | 13 |
| 4 | 275 | 13 | 4 | 276 | 13 |
| 5 | 275 | 13 | 5 | 276 | 13 |
| 6 | 275 | 13 | 6 | 276 | 13 |
| 249 | 275 | 13 | 249 | 276 | 13 |
| 255 | 275 | 13 | 255 | 276 | 13 |
| 256 | 275 | 13 | 256 | 276 | 13 |
| 502 | 275 | 13 | 502 | 276 | 13 |
| 1 | 275 | 14 | 1 | 276 | 14 |
| 2 | 275 | 14 | 2 | 276 | 14 |
| 3 | 275 | 14 | 3 | 276 | 14 |
| 4 | 275 | 14 | 4 | 276 | 14 |
| 5 | 275 | 14 | 5 | 276 | 14 |
| 6 | 275 | 14 | 6 | 276 | 14 |
| 249 | 275 | 14 | 249 | 276 | 14 |
| 255 | 275 | 14 | 255 | 276 | 14 |
| 256 | 275 | 14 | 256 | 276 | 14 |
| 502 | 275 | 14 | 502 | 276 | 14 |
| 1 | 275 | 15 | 1 | 276 | 15 |
| 2 | 275 | 15 | 2 | 276 | 15 |
| 3 | 275 | 15 | 3 | 276 | 15 |
| 4 | 275 | 15 | 4 | 276 | 15 |
| 5 | 275 | 15 | 5 | 276 | 15 |
| 6 | 275 | 15 | 6 | 276 | 15 |
| 249 | 275 | 15 | 249 | 276 | 15 |
| 255 | 275 | 15 | 255 | 276 | 15 |
| 256 | 275 | 15 | 256 | 276 | 15 |
| 502 | 275 | 15 | 502 | 276 | 15 |
| 1 | 275 | 257 | 1 | 276 | 257 |
| 2 | 275 | 257 | 2 | 276 | 257 |
| 3 | 275 | 257 | 3 | 276 | 257 |
| 4 | 275 | 257 | 4 | 276 | 257 |
| 5 | 275 | 257 | 5 | 276 | 257 |
| 6 | 275 | 257 | 6 | 276 | 257 |
| 249 | 275 | 257 | 249 | 276 | 257 |
| 255 | 275 | 257 | 255 | 276 | 257 |
| 256 | 275 | 257 | 256 | 276 | 257 |
| 502 | 275 | 257 | 502 | 276 | 257 |
| 1 | 275 | 259 | 1 | 276 | 259 |
| 2 | 275 | 259 | 2 | 276 | 259 |
| 3 | 275 | 259 | 3 | 276 | 259 |
| 4 | 275 | 259 | 4 | 276 | 259 |
| 5 | 275 | 259 | 5 | 276 | 259 |
| 6 | 275 | 259 | 6 | 276 | 259 |
| 249 | 275 | 259 | 249 | 276 | 259 |
| 255 | 275 | 259 | 255 | 276 | 259 |
| 256 | 275 | 259 | 256 | 276 | 259 |
| 502 | 275 | 259 | 502 | 276 | 259 |
| 1 | 275 | 260 | 1 | 276 | 260 |
| 2 | 275 | 260 | 2 | 276 | 260 |
| 3 | 275 | 260 | 3 | 276 | 260 |
| 4 | 275 | 260 | 4 | 276 | 260 |
| 5 | 275 | 260 | 5 | 276 | 260 |
| 6 | 275 | 260 | 6 | 276 | 260 |
| 249 | 275 | 260 | 249 | 276 | 260 |
| 255 | 275 | 260 | 255 | 276 | 260 |
| 256 | 275 | 260 | 256 | 276 | 260 |
| 502 | 275 | 260 | 502 | 276 | 260 |
| 1 | 275 | 261 | 1 | 276 | 261 |
| 2 | 275 | 261 | 2 | 276 | 261 |
| 3 | 275 | 261 | 3 | 276 | 261 |
| 4 | 275 | 261 | 4 | 276 | 261 |
| 5 | 275 | 261 | 5 | 276 | 261 |
| 6 | 275 | 261 | 6 | 276 | 261 |
| 249 | 275 | 261 | 249 | 276 | 261 |
| 255 | 275 | 261 | 255 | 276 | 261 |
| 256 | 275 | 261 | 256 | 276 | 261 |
| 502 | 275 | 261 | 502 | 276 | 261 |
| 1 | 275 | 503 | 1 | 276 | 503 |
| 2 | 275 | 503 | 2 | 276 | 503 |
| 3 | 275 | 503 | 3 | 276 | 503 |
| 4 | 275 | 503 | 4 | 276 | 503 |
| 5 | 275 | 503 | 5 | 276 | 503 |
| 6 | 275 | 503 | 6 | 276 | 503 |
| 249 | 275 | 503 | 249 | 276 | 503 |
| 255 | 275 | 503 | 255 | 276 | 503 |
| 256 | 275 | 503 | 256 | 276 | 503 |
| 502 | 275 | 503 | 502 | 276 | 503 |
| 1 | 275 | 504 | 1 | 276 | 504 |
| 2 | 275 | 504 | 2 | 276 | 504 |
| 3 | 275 | 504 | 3 | 276 | 504 |
| 4 | 275 | 504 | 4 | 276 | 504 |
| 5 | 275 | 504 | 5 | 276 | 504 |
| 6 | 275 | 504 | 6 | 276 | 504 |
| 249 | 275 | 504 | 249 | 276 | 504 |
| 255 | 275 | 504 | 255 | 276 | 504 |
| 256 | 275 | 504 | 256 | 276 | 504 |
| 502 | 275 | 504 | 502 | 276 | 504 |
| 1 | 275 | 507 | 1 | 276 | 507 |
| 2 | 275 | 507 | 2 | 276 | 507 |
| 3 | 275 | 507 | 3 | 276 | 507 |
| 4 | 275 | 507 | 4 | 276 | 507 |
| 5 | 275 | 507 | 5 | 276 | 507 |
| 6 | 275 | 507 | 6 | 276 | 507 |
| 249 | 275 | 507 | 249 | 276 | 507 |
| 255 | 275 | 507 | 255 | 276 | 507 |
| 256 | 275 | 507 | 256 | 276 | 507 |
| 502 | 275 | 507 | 502 | 276 | 507 |
| 1 | 275 | 550 | 1 | 276 | 550 |
| 2 | 275 | 550 | 2 | 276 | 550 |
| 3 | 275 | 550 | 3 | 276 | 550 |
| 4 | 275 | 550 | 4 | 276 | 550 |
| 5 | 275 | 550 | 5 | 276 | 550 |
| 6 | 275 | 550 | 6 | 276 | 550 |
| 249 | 275 | 550 | 249 | 276 | 550 |
| 255 | 275 | 550 | 255 | 276 | 550 |
| 256 | 275 | 550 | 256 | 276 | 550 |
| 502 | 275 | 550 | 502 | 276 | 550 |
| 1 | 275 | 551 | 1 | 276 | 551 |
| 2 | 275 | 551 | 2 | 276 | 551 |
| 3 | 275 | 551 | 3 | 276 | 551 |
| 4 | 275 | 551 | 4 | 276 | 551 |
| 5 | 275 | 551 | 5 | 276 | 551 |
| 6 | 275 | 551 | 6 | 276 | 551 |
| 249 | 275 | 551 | 249 | 276 | 551 |
| 255 | 275 | 551 | 255 | 276 | 551 |
| 256 | 275 | 551 | 256 | 276 | 551 |
| 502 | 275 | 551 | 502 | 276 | 551 |
| 1 | 275 | 552 | 1 | 276 | 552 |
| 2 | 275 | 552 | 2 | 276 | 552 |
| 3 | 275 | 552 | 3 | 276 | 552 |
| 4 | 275 | 552 | 4 | 276 | 552 |
| 5 | 275 | 552 | 5 | 276 | 552 |
| 6 | 275 | 552 | 6 | 276 | 552 |
| 249 | 275 | 552 | 249 | 276 | 552 |
| 255 | 275 | 552 | 255 | 276 | 552 |
| 256 | 275 | 552 | 256 | 276 | 552 |
| 502 | 275 | 552 | 502 | 276 | 552 |
| 1 | 275 | 553 | 1 | 276 | 553 |
| 2 | 275 | 553 | 2 | 276 | 553 |
| 3 | 275 | 553 | 3 | 276 | 553 |
| 4 | 275 | 553 | 4 | 276 | 553 |
| 5 | 275 | 553 | 5 | 276 | 553 |
| 6 | 275 | 553 | 6 | 276 | 553 |
| 249 | 275 | 553 | 249 | 276 | 553 |
| 255 | 275 | 553 | 255 | 276 | 553 |
| 256 | 275 | 553 | 256 | 276 | 553 |
| 502 | 275 | 553 | 502 | 276 | 553 |
| 1 | 275 | 554 | 1 | 276 | 554 |
| 2 | 275 | 554 | 2 | 276 | 554 |
| 3 | 275 | 554 | 3 | 276 | 554 |
| 4 | 275 | 554 | 4 | 276 | 554 |
| 5 | 275 | 554 | 5 | 276 | 554 |
| 6 | 275 | 554 | 6 | 276 | 554 |
| 249 | 275 | 554 | 249 | 276 | 554 |
| 255 | 275 | 554 | 255 | 276 | 554 |
| 256 | 275 | 554 | 256 | 276 | 554 |
| 502 | 275 | 554 | 502 | 276 | 554 |
| 1 | 275 | 555 | 1 | 276 | 555 |
| 2 | 275 | 555 | 2 | 276 | 555 |
| 3 | 275 | 555 | 3 | 276 | 555 |
| 4 | 275 | 555 | 4 | 276 | 555 |
| 5 | 275 | 555 | 5 | 276 | 555 |
| 6 | 275 | 555 | 6 | 276 | 555 |
| 249 | 275 | 555 | 249 | 276 | 555 |
| 255 | 275 | 555 | 255 | 276 | 555 |
| 256 | 275 | 555 | 256 | 276 | 555 |
| 502 | 275 | 555 | 502 | 276 | 555 |
| 1 | 275 | 556 | 1 | 276 | 556 |
| 2 | 275 | 556 | 2 | 276 | 556 |
| 3 | 275 | 556 | 3 | 276 | 556 |
| 4 | 275 | 556 | 4 | 276 | 556 |
| 5 | 275 | 556 | 5 | 276 | 556 |
| 6 | 275 | 556 | 6 | 276 | 556 |
| 249 | 275 | 556 | 249 | 276 | 556 |
| 255 | 275 | 556 | 255 | 276 | 556 |
| 256 | 275 | 556 | 256 | 276 | 556 |
| 502 | 275 | 556 | 502 | 276 | 556 |
| 1 | 275 | 557 | 1 | 276 | 557 |
| 2 | 275 | 557 | 2 | 276 | 557 |
| 3 | 275 | 557 | 3 | 276 | 557 |
| 4 | 275 | 557 | 4 | 276 | 557 |
| 5 | 275 | 557 | 5 | 276 | 557 |
| 6 | 275 | 557 | 6 | 276 | 557 |
| 249 | 275 | 557 | 249 | 276 | 557 |
| 255 | 275 | 557 | 255 | 276 | 557 |
| 256 | 275 | 557 | 256 | 276 | 557 |
| 502 | 275 | 557 | 502 | 276 | 557 |
| 1 | 275 | 558 | 1 | 276 | 558 |
| 2 | 275 | 558 | 2 | 276 | 558 |
| 3 | 275 | 558 | 3 | 276 | 558 |
| 4 | 275 | 558 | 4 | 276 | 558 |
| 5 | 275 | 558 | 5 | 276 | 558 |
| 6 | 275 | 558 | 6 | 276 | 558 |
| 249 | 275 | 558 | 249 | 276 | 558 |
| 255 | 275 | 558 | 255 | 276 | 558 |
| 256 | 275 | 558 | 256 | 276 | 558 |
| 502 | 275 | 558 | 502 | 276 | 558 |

In some embodiments, there are additional amino acids between the N-terminal peptide and the linker peptide. In some embodiments, there are additional amino acids between the C-terminal peptide and the linker peptide. In some embodiments, there are no additional amino acids between the N-terminal peptide and the linker peptide. In some embodiments, there are no additional amino acids between the C-terminal peptide and the linker peptide.

In some embodiments, the portion of the fusion protein comprising the N-terminal peptide, the linker peptide, and the C-terminal peptide comprises or consists of the amino acid sequences shown in Table 5, below.

TABLE 5
Peptide Combinations for the Fusion Protein

| SEQ ID NO: | Amino Acid Sequence |
|---|---|
| 28 | DSWQEEVIKLCGRELVRAQIAICGKSTASDAAGADANAGARQLYSALANK |
| | CCHVGCTKRSLAQFC |
| 29 | DSWQEEVIKLCGRELVRAQIAICGKSTASDAAGANADAGARQLYSALANK |
| | CCHVGCTKRSLAQFC |
| 30 | DSWQEEVIKLCGRELVRAQIAICGKSTASDAAGADADAGARQLYSALANK |
| | CCHVGCTKRSLAQFC |
| 31 | DSWQEEVIKLCGRELVRAQIAICGKSTASDAAGAEAEAGARQLYSALANK |
| | CCHVGCTKRSLAQFC |
| 32 | DSWQEEVIKLCGRELVRAQIAICGKSTASDAAGAQADAGARQLYSALANK |
| | CCHVGCTKRSLAQFC |
| 33 | DSWQEEVIKLCGRELVRAQIAICGKSTASDAAGADAQAGARQLYSALANK |
| | CCHVGCTKRSLAQFC |
| 34 | DSWQEEVIKLCGRELVRAQIAICGKSTASDAAGAQAQAGARQLYSALANK |
| | CCHVGCTKRSLAQFC |
| 35 | DSWQEEVIKLCGRELVRAQIAICGQSTASDAAGADANAGARQLYSALANK |
| | CCHVGCTKRSLAQFC |
| 36 | DSWQEEVIKLCGRELVRAQIAICGQSTASDAAGANADAGARQLYSALANK |
| | CCHVGCTKRSLAQFC |
| 37 | DSWQEEVIKLCGRELVRAQIAICGQSTASDAAGADADAGARQLYSALANK |
| | CCHVGCTKRSLAQFC |
| 38 | DSWQEEVIKLCGRELVRAQIAICGQSTASDAAGAEAEAGARQLYSALANK |
| | CCHVGCTKRSLAQFC |
| 39 | DSWQEEVIKLCGRELVRAQIAICGQSTASDAAGAQADAGARQLYSALANK |
| | CCHVGCTKRSLAQFC |
| 40 | DSWQEEVIKLCGRELVRAQIAICGQSTASDAAGADAQAGARQLYSALANK |
| | CCHVGCTKRSLAQFC |
| 41 | DSWQEEVIKLCGRELVRAQIAICGQSTASDAAGAQAQAGARQLYSALANK |
| | CCHVGCTKRSLAQFC |
| 42 | DSWQEEVIKLCGRELVRAQIAICGKSTASDAAGADANAGARQLYSALANK |
| | CCHVGCTKQSLAQFC |
| 43 | DSWQEEVIKLCGRELVRAQIAICGKSTASDAAGANADAGARQLYSALANK |
| | CCHVGCTKQSLAQFC |
| 44 | DSWQEEVIKLCGRELVRAQIAICGKSTASDAAGADADAGARQLYSALANK |
| | CCHVGCTKQSLAQFC |
| 45 | DSWQEEVIKLCGRELVRAQIAICGKSTASDAAGAEAEAGARQLYSALANK |
| | CCHVGCTKQSLAQFC |
| 46 | DSWQEEVIKLCGRELVRAQIAICGKSTASDAAGAQADAGARQLYSALANK |
| | CCHVGCTKQSLAQFC |
| 47 | DSWQEEVIKLCGRELVRAQIAICGKSTASDAAGADAQAGARQLYSALANK |
| | CCHVGCTKQSLAQFC |
| 48 | DSWQEEVIKLCGRELVRAQIAICGKSTASDAAGAQAQAGARQLYSALANK |
| | CCHVGCTKQSLAQFC |
| 49 | DSWQEEVIKLCGRELVRAQIAICGQSTASDAAGADANAGARQLYSALANK |
| | CCHVGCTKQSLAQFC |
| 50 | DSWQEEVIKLCGRELVRAQIAICGQSTASDAAGANADAGARQLYSALANK |
| | CCHVGCTKQSLAQFC |
| 51 | DSWQEEVIKLCGRELVRAQIAICGQSTASDAAGADADAGARQLYSALANK |
| | CCHVGCTKQSLAQFC |
| 52 | DSWQEEVIKLCGRELVRAQIAICGQSTASDAAGAEAEAGARQLYSALANK |
| | CCHVGCTKQSLAQFC |
| 53 | DSWQEEVIKLCGRELVRAQIAICGQSTASDAAGAQADAGARQLYSALANK |
| | CCHVGCTKQSLAQFC |
| 54 | DSWQEEVIKLCGRELVRAQIAICGQSTASDAAGADAQAGARQLYSALANK |
| | CCHVGCTKQSLAQFC |
| 55 | DSWQEEVIKLCGRELVRAQIAICGQSTASDAAGAQAQAGARQLYSALANK |
| | CCHVGCTKQSLAQFC |
| 56 | DSYQEEVIKLCGRELVRAQIAICGKSTASDAAGAEAEAGARQLYSALANK |
| | CCHVGCTKRSLAQFC |
| 57 | DSFQEEVIKLCGRELVRAQIAICGKSTASDAAGAEAEAGARQLYSALANK |
| | CCHVGCTKRSLAQFC |
| 58 | DSLQEEVIKLCGRELVRAQIAICGKSTASDAAGAEAEAGARQLYSALANK |
| | CCHVGCTKRSLAQFC |
| 59 | DSIQEEVIKLCGRELVRAQIAICGKSTASDAAGAEAEAGARQLYSALANK |
| | CCHVGCTKRSLAQFC |
| 60 | DSWQEEVIKLCGRELVRAQIAICGKSTASDAAGAEAEAGARQLYSALANK |
| | CCYVGCTKRSLAQFC |
| 61 | DSWQEEVIKLCGRELVRAQIAICGKSTASDAAGAEAEAGARQLYSALANK |
| | CCLVGCTKRSLAQFC |
| 62 | DSWQEEVIKLCGRELVRAQIAICGKSTASDAAGAEAEAGARQLYSALANK |
| | CCQVGCTKRSLAQFC |
| 63 | DSWQEEVIKLCGRELVRAQIAICGKSTASDAAGAEAEAGARQLYSALANK |
| | CCKVGCTKRSLAQFC |
| 64 | DSYQEEVIKLCGRELVRAQIAICGKSTASDAAGAEAEAGARQLYSALANK |
| | CCYVGCTKRSLAQFC |
| 65 | DSYQEEVIKLCGRELVRAQIAICGKSTASDAAGAEAEAGARQLYSALANK |
| | CCKVGCTKRSLAQFC |
| 66 | DSYQEEVIKLCGRELVRAQIAICGKSTGGEGSGGEGEGGGRQLYSALANK |
| | CCYVGCTKRSLAQFC |
| 67 | DSYQEEVIKLCGRELVRAQIAICGKSTGGEGSGGEGEGGGRQLYSALANK |
| | CCKVGCTKRSLAQFC |
| 68 | DSYQEEVIKLCGRELVRAQIAICGKSTGGEGSGGEGSGGGRQLYSALANK |
| | CCYVGCTKRSLAQFC |
| 69 | DSYQEEVIKLCGRELVRAQIAICGKSTGGEGSGGEGSGGGRQLYSALANK |
| | CCKVGCTKRSLAQFC |
| 70 | DSYQEEVIKLCGRELVRAQIAICGKSTASDAAGAEAEAGARQLYSALANK |
| | CCYVGCTKQSLAQFC |
| 71 | DSYQEEVIKLCGRELVRAQIAICGKSTASDAAGAEAEAGARQLYSALANK |
| | CCKVGCTKQSLAQFC |
| 72 | DSYQEEVIKLCGRELVRAQIAICGKSTGGEGSGGEGEGGGRQLYSALANK |
| | CCYVGCTKQSLAQFC |
| 73 | DSYQEEVIKLCGRELVRAQIAICGKSTGGEGSGGEGEGGGRQLYSALANK |
| | CCKVGCTKQSLAQFC |
| 74 | DSYQEEVIKLCGRELVRAQIAICGKSTGGEGSGGEGSGGGRQLYSALANK |
| | CCYVGCTKQSLAQFC |
| 75 | DSYQEEVIKLCGRELVRAQIAICGKSTGGEGSGGEGSGGGRQLYSALANK |
| | CCKVGCTKQSLAQFC |
| 278 | DSWKEEVIKLCGRELVRAQIAICGKSTGGGEGGGEGGGEGRQLYSALANK |
| | CCHVGCTKRSLARFC |
| 279 | DSWKEEVIKLCGRELVRAQIAICGKSTGGGEGGGEGGGEGGGQLYSALAN |
| | KCCHVGCTKRSLARFC |
| 280 | DSWMEEVIKLCGRELVRAQIAICGKSTGGGEGGGEGGGEGRQLYSALANK |
| | CCHVGCTKRSLARFC |
| 281 | DSWQEEVIKLCGRELVRAQIAICGKSTGGGEGGGEGGGEGRQLYSALANK |
| | CCHVGCTKRSLARFC |
| 282 | DSWMEEVIKLCGRELVRAQIAICGKSTGGGEGGGEGGGEGGGQLYSALAN |
| | KCCHVGCTKRSLARFC |
| 283 | DSWQEEVIKLCGRELVRAQIAICGKSTGGGEGGGEGGGEGGGQLYSALAN |
| | KCCHVGCTKRSLARFC |
| 284 | DSWMEEVIKLCGRELVRAQIAICGKSTGGEGGGEEGGGEGGQLYSALANK |
| | CCHVGCTKRSLARFC |
| 285 | DSWQEEVIKLCGRELVRAQIAICGKSTGGEGGGEEGGGEGGQLYSALANK |
| | CCHVGCTKRSLARFC |
| 286 | DSWMEEVIKLCGRELVRAQIAICGKSTGGEGGGEEGGGEGGQLYSALANK |
| | CCHVGCTKRSLARFC |
| 287 | DSWQEEVIKLCGRELVRAQIAICGKSTGGEGGGEEGGGEGGQLYSALANK |
| | CCHVGCTKRSLARFC |
| 288 | DSWQEEVIKLCGRELVRAQIAICGKSTGGGEGGGEGGGEGRQLYSALANK |
| | CCHVGCTKRSLAQFC |
| 289 | DSWQEEVIKLCGRELVRAQIAICGKSTGGGEGGGEGGGEGRQLYSALANK |
| | CCHVGCTKRSLARFC |
| 290 | DSWQEEVIKLCGRELVRAQIAICGKSTGGGEGGGEGGGEGRQLYSALANK |
| | CCHVGCTKRSLAQFC |
| 291 | DSWQEEVIKLCGRELVRAQIAICGKSTGGGEEGGGEEGGGRQLYSALANK |
| | CCHVGCTKRSLARFC |
| 292 | DSWQEEVIKLCGRELVRAQIAICGKSTGGGEEGGGEEGGGRQLYSALANK |
| | CCHVGCTKRSLAQFC |
| 293 | DSWQEEVIKLCGRELVRAQIAICGKSTGGGEEGGGEEGGGRQLYSALANK |
| | CCHVGCTKRSLARFC |
| 294 | DSWQEEVIKLCGRELVRAQIAICGKSTGGGEEGGGEEGGGRQLYSALANK |
| | CCHVGCTKRSLAQFC |
| 295 | DSWQEEVIKLCGRELVRAQIAICGKSTGGEGEGGEGEGGSRQLYSALANK |
| | CCHVGCTKRSLAQFC |
| 296 | DSWQEEVIKLCGRELVRAQIAICGKSTGGEGEGGEGEGGSRQLYSALANK |
| | CCHVGCTKRSLARFC |
| 297 | DSWQEEVIKLCGRELVRAQIAICGKSTGGEGEGGEGEGGSRQLYSALANK |
| | CCHVGCTKRSLAQFC |
| 508 | DSYQEEVIKLCGRELVRAQIAICGKSTGGEGSGGEGEGGGRQLYSALANK |
| | CCHVGCTKRSLAQFC |
| 509 | DSYQEEVIKLCGRELVRAQIAICGKSTGGEGSGGEGEGGGRQLYSALANK |
| | CCQVGCTKRSLAQFC |
| 510 | DSYQEEVIKLCGRELVRAQIAICGKSTGGEGSGGEGEGGGRQLYSALANK |
| | CCHVGCTKQSLAQFC |
| 511 | DSYQEEVIKLCGRELVRAQIAICGKSTGGEGSGGEGEGGGRQLYSALANK |
| | CCQVGCTKQSLAQFC |
| 512 | DSYQEEVIKLCGRELVRAQIAICGKSTGGEGSGGEGSGGGRQLYSALANK |
| | CCHVGCTKRSLAQFC |
| 513 | DSYQEEVIKLCGRELVRAQIAICGKSTGGEGSGGEGSGGGRQLYSALANK |
| | CCQVGCTKRSLAQFC |
| 514 | DSYQEEVIKLCGRELVRAQIAICGKSTGGEGSGGEGSGGGRQLYSALANK |
| | CCHVGCTKQSLAQFC |
| 515 | DSYQEEVIKLCGRELVRAQIAICGKSTGGEGSGGEGSGGGRQLYSALANK |
| | CCQVGCTKQSLAQFC |

### IgG Fc

In some embodiments, the fusion proteins provided herein further comprise an IgG Fc (or Fc region). As used herein, the term “IgG Fc” or “Fc region” refers to the portion of an immunoglobulin formed by the Fc domains of its two heavy chains. The Fc region can be a wild-type Fc region (native Fc region) or a variant Fc region. A native Fc region is homodimeric. In some embodiments, the fusion proteins provided herein form dimers (e.g., homodimers via interaction between Fc regions. In some embodiments, two fusion proteins are linked into a dimer (e.g., a homodimer) via 2 hinge region interchain disulfide bonds between the Fc regions of each fusion protein (e.g., at the N-terminus). In some embodiments, the Fc region comprises one intrachain disulfide bond in the CH2 domain and one intrachain disulfide bond in the CH3 domain.

The Fc region of the fusion proteins provided herein can be derived from any native immunoglobulin. In some embodiments, the Fc region is formed from an IgA, IgD, IgE, or IgG heavy chain constant region. In some embodiments, the Fc region is formed from an IgG heavy chain constant region. In some embodiments, the IgG heavy chain is an IgG1, IgG2, IgG3 or IgG4 heavy chain constant region. In some embodiments, the Fc region is formed from an IgG1 heavy chain constant region. In some embodiments, the IgG1 heavy chain constant region comprises a G1m1(a), G1m2(x), G1m3(f), or G1m17(z) allotype. See, e.g., Jefferis and Lefranc (2009) mAbs 1(4): 332-338, and de Taeye et al. (2020) Front Immunol. 11:740, incorporated herein by reference in their entirety. The IgG Fc can be linked to the N-terminal end of the N-terminal peptide or the C-terminal end of the C-terminal peptide. The IgG Fc can be linked directly to the N-terminal peptide or the C-terminal peptide or they can be linked to the N-terminal peptide or the C-terminal peptide through an IgG Fc linker. In some embodiments, the IgG Fc linker comprises or consists of 1, 2, 3, 4, 5, 6, 7, 8, 9, or 10 amino acids. In some embodiments, the IgG Fc linker comprises or consists of 1, 2, 3, 4, or 5 amino acids. In some embodiments, the IgG Fc linker comprises or consists of 3 or 4 amino acids. In some embodiments, the IgG Fc linker comprises or consists of the amino acid sequence of GGS. In some embodiments, the IgG Fc linker comprises or consists of the amino acid sequence of EGGS (SEQ ID NO: 299).

In some embodiments, the IgG Fc comprises a C-terminal lysine (K). It is known in the art that the C-terminal lysine (K) in many monoclonal antibodies is flexible, and is often clipped off during expression and purification with no known impairment in activity. In some embodiments, the C-terminal lysine (K) is replaced with a C-terminal glutamic acid (E). As such, in some embodiments, the IgG Fc comprises a C-terminal glutamic acid (E).

In some embodiments, the IgG Fc comprises the amino acid sequence of one of SEQ ID NOs: 76-83 with GGS as the IgG Fc linker at the C-terminal end of the IgG Fc. In some embodiments, the IgG Fc comprises the amino acid sequence of one of SEQ ID NOs: 76-83 with SEQ ID NO: 299 as the IgG Fc linker at the C-terminal end of the IgG Fc.

In some embodiments, one, two, or more mutations (e.g., amino acid substitutions) are introduced into the Fc region of an antibody described herein (e.g., CH2 domain (residues 231-340 of human IgG1) and/or CH3 domain (residues 341-447 of human IgG1)) and/or the hinge region, numbered according to the EU numbering system, to alter one or more functional properties of the antibody, such as serum half-life, complement fixation, Fc receptor binding, and/or antigen-dependent cellular cytotoxicity.

In certain embodiments, one, two, or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425, herein incorporated by reference in its entirety. The number of cysteine residues in the hinge region of the CH1 domain may be altered to, e.g., facilitate assembly of the light and heavy chains, or to alter (e.g., increase or decrease) the stability of the antibody.

In a specific embodiment, one, two, or more amino acid mutations (e.g., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375, and 6,165,745, all of which are herein incorporated by reference in their entireties, for examples of mutations that will alter (e.g., decrease or increase) the half-life of an antibody in vivo. In certain embodiments, one, two, or more amino acid mutations (e.g., substitutions, insertions, or deletions) are introduced into an IgG constant domain, or FcRn-binding fragment thereof (preferably an Fc or hinge-Fc domain fragment) to decrease the half-life of the antibody in vivo. In other embodiments, one, two, or more amino acid mutations (e.g., substitutions, insertions, or deletions) are introduced into an IgG constant domain, or FcRn-binding fragment thereof (preferably an Fc or hinge-Fc domain fragment) to increase the half-life of the antibody in vivo. In a specific embodiment, the antibodies may have one or more amino acid mutations (e.g., substitutions) in the second constant (CH2) domain (residues 231-340 of human IgG1) and/or the third constant (CH3) domain (residues 341-447 of human IgG1), numbered according to the EU numbering system. In a specific embodiment, the constant region of the IgG1 of an antibody described herein comprises a methionine (M) to tyrosine (Y) substitution in position 252, a serine(S) to threonine (T) substitution in position 254, and a threonine (T) to glutamic acid (E) substitution in position 256, numbered according to the EU numbering system. See, U.S. Pat. No. 7,658,921, which is herein incorporated by reference in its entirety. This type of mutant IgG, referred to as “YTE mutant” has been shown to display fourfold increased half-life as compared to wild-type versions of the same antibody (see, Dall'Acqua W F et al., (2006) J Biol Chem 281:23514-24, which is herein incorporated by reference in its entirety). In certain embodiments, an antibody comprises an IgG constant domain comprising one, two, three or more amino acid substitutions of amino acid residues at positions 251-257, 285-290, 308-314, 385-389, and 428-436, numbered according to the EU numbering system.

In certain embodiments, one, two, or more mutations (e.g., amino acid substitutions) are introduced into the Fc region of an antibody described herein (e.g., CH2 domain (residues 231-340 of human IgG1) and/or CH3 domain (residues 341-447 of human IgG1)) and/or the hinge region, numbered according to the EU numbering system, to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication Nos. WO 02/060919; WO 98/23289; and WO 97/34631, all of which are herein incorporated by reference in their entireties.

In certain embodiments, the antibody comprises a heavy chain constant region that is a variant of a wild-type heavy chain constant region, wherein the variant heavy chain constant region binds to FcγRIIB with higher affinity than the wild-type heavy chain constant region binds to FcγRIIB. In certain embodiments, the variant heavy chain constant region is a variant human heavy chain constant region, e.g., a variant human IgG1, a variant human IgG2, or a variant human IgG4 heavy chain constant region. In certain embodiments, the variant human IgG heavy chain constant region comprises one or more of the following amino acid mutations, according to the EU numbering system: G236D, P238D, S239D, S267E, L328F, and L328E. In certain embodiments, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F, according to the EU numbering system. In certain embodiments, the FcγRIIB is expressed on a cell selected from the group consisting of macrophages, monocytes, B cells, dendritic cells, endothelial cells, and activated T cells.

In a further embodiment, one, two, or more amino acid substitutions are introduced into an IgG constant domain Fc region to alter the effector function(s) of the antibody. For example, one or more amino acids selected from amino acid residues 234, 235, 236, 237, 239, 243, 267, 292, 297, 300, 318, 320, 322, 328, 330, 332, and 396, numbered according to the EU numbering system, can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, each of which is herein incorporated by reference in its entirety. In certain embodiments, the deletion or inactivation (through point mutations or other means) of a constant region domain may reduce Fc receptor binding of the circulating antibody thereby increasing tumor localization. See, e.g., U.S. Pat. Nos. 5,585,097 and 8,591,886, each of which is herein incorporated by reference in its entirety, for a description of mutations that delete or inactivate the constant domain and thereby increase tumor localization. In certain embodiments, one or more amino acid substitutions may be introduced into the Fc region of an antibody described herein to remove potential glycosylation sites on the Fc region, which may reduce Fc receptor binding (see, e.g., Shields R L et al., (2001) J Biol Chem 276:6591-604, which is herein incorporated by reference in its entirety). In various embodiments, one or more of the following mutations in the constant region of an antibody described herein may be made: an N297A substitution; an N297Q substitution; an L234A substitution; an L234F substitution; an L235A substitution; an L235F substitution; an L235V substitution; an L237A substitution; an S239D substitution; an E233P substitution; an L234V substitution; an L235A substitution; a C236 deletion; a P238A substitution; an S239D substitution; an F243L substitution; a D265A substitution; an S267E substitution; an L328F substitution; an R292P substitution; a Y300L substitution; an A327Q substitution; a P329A substitution (PA); an A332L substitution; an 1332E substitution; or a P396L substitution, numbered according to the EU numbering system.

In certain embodiments, a mutation selected from the group consisting of D265A, P329A, and a combination thereof, numbered according to the EU numbering system, may be made in the constant region of an antibody described herein. In certain embodiments, a mutation selected from the group consisting of L235A, L237A, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein. In certain embodiments, a mutation selected from the group consisting of S239D, 1332E, optionally A330L, and a combination thereof, numbered according to the EU numbering system, may be made in the constant region of an antibody described herein. In certain embodiments, a mutation selected from the group consisting of L235V, F243L, R292P, Y300L, P396L, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein.

In a specific embodiment, an antibody described herein comprises the constant domain of an IgG1 with an N297Q or N297A amino acid substitution, numbered according to the EU numbering system. In one embodiment, an antibody described herein comprises the constant domain of an IgG1 with a mutation selected from the group consisting of D265A, P329A, and a combination thereof, numbered according to the EU numbering system. In another embodiment, an antibody described herein comprises the constant domain of an IgG1 with a mutation selected from the group consisting of L234A, L235A (LALA), and a combination thereof, numbered according to the EU numbering system. In another embodiment, an antibody described herein comprises the constant domain of an IgG1 with a mutation selected from the group consisting of L234F, L235F, N297A, and a combination thereof, numbered according to the EU numbering system. In certain embodiments, amino acid residues in the constant region of an antibody described herein in the positions corresponding to positions L234, L235, and D265 in a human IgG1 heavy chain, numbered according to the EU numbering system, are not L, L, and D, respectively. This approach is described in detail in International Publication No. WO 14/108483, which is herein incorporated by reference in its entirety. In a particular embodiment, the amino acids corresponding to positions L234, L235, and D265 in a human IgG1 heavy chain are F, E, and A; or A, A, and A, respectively, numbered according to the EU numbering system.

In certain embodiments, one or more amino acids selected from amino acid residues 329, 331, and 322 in the constant region of an antibody described herein, numbered according to the EU numbering system, can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al.), which is herein incorporated by reference in its entirety. In certain embodiments, one or more amino acid residues within amino acid positions 231 to 238 in the N-terminal region of the CH2 domain of an antibody described herein are altered to thereby alter the ability of the antibody to fix complement, numbered according to the EU numbering system. This approach is described further in International Publication No. WO 94/29351, which is herein incorporated by reference in its entirety. In certain embodiments, the Fc region of an antibody described herein is modified to increase the ability of the antibody to mediate antibody dependent cellular cytotoxicity (ADCC) and/or to increase the affinity of the antibody for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU numbering system. This approach is described further in International Publication No. WO 00/42072, which is herein incorporated by reference in its entirety.

In some embodiments, the IgG Fc is an IgG1 Fc, or a derivative thereof. In some embodiments, the IgG Fc or IgG1 Fc comprises an amino acid sequence at least 85, 90, 95, 96, 97, 98, or 99% identical to the amino acid sequence of IgG1 Fc. In some embodiments, the IgG Fc or IgG1 Fc comprises an amino acid sequence at least 85, 90, 95, 96, 97, 98, 99, or 100% identical to an amino acid sequence provided below in Table 6.

TABLE 6
IgG Fc Amino Acid Sequences

| SEQ | | |
|---|---|---|
| ID | | |
| NO: | Description | Sequence |
| 76 | IgG1 Fc | DKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 77 | IgG1 Fc | DKTHTCPPCPAPEAAGGPSVFLFPPKPKDTLMISRTPEVTCVVVDV |
| | LALA | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 78 | IgG1 Fc | DKTHTCPPCPAPEAAGGPSVFLFPPKPKDTLMISRTPEVTCVVVDV |
| | LALA PA | SHEDPEVKFNWYVDGVEVHNAKTKPREEQYNSTYRVVSVLTVLHQD |
| | | WLNGKEYKCKVSNKALAAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 79 | IgG1 Fc | DKTHTCPPCPAPEAAGGPSVFLFPPKPKDTLMISRTPEVTCVVVDV |
| | LALA PA | SHEDPEVKFNWYVDGVEVHNAKTKPREEQYNSTYRVVSVLTVLHQD |
| | LS | WLNGKEYKCKVSNKALAAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVLHEALHSHYTQKSLSLSPGK |
| 80 | IgG1 Fc | DKTHTCPPCPAPELLGGPSVFLFPPKPKDTLMISRTPEVTCVVVDV |
| | without C- | SHEDPEVKFNWYVDGVEVHNAKTKPREEQYNSTYRVVSVLTVLHQD |
| | terminal | WLNGKEYKCKVSNKALPAPIEKTISKAKGQPREPQVYTLPPSRDEL |
| | lysine | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPG |
| 81 | IgG1 Fc | DKTHTCPPCPAPEAAGGPSVFLFPPKPKDTLMISRTPEVTCVVVDV |
| | LALA | SHEDPEVKFNWYVDGVEVHNAKTKPREEQYNSTYRVVSVLTVLHQD |
| | without C- | WLNGKEYKCKVSNKALPAPIEKTISKAKGQPREPQVYTLPPSRDEL |
| | terminal | TKNQVSLTCLVKGFYPSDIAVEWESNGQPENNYKTTPPVLDSDGSF |
| | lysine | FLYSKLTVDKSRWQQGNVFSCSVMHEALHNHYTQKSLSLSPG |
| 82 | IgG1 Fc | DKTHTCPPCPAPEAAGGPSVFLFPPKPKDTLMISRTPEVTCVVVDV |
| | LALA PA | SHEDPEVKFNWYVDGVEVHNAKTKPREEQYNSTYRVVSVLTVLHQD |
| | without C- | WLNGKEYKCKVSNKALAAPIEKTISKAKGQPREPQVYTLPPSRDEL |
| | terminal | TKNQVSLTCLVKGFYPSDIAVEWESNGQPENNYKTTPPVLDSDGSF |
| | lysine | FLYSKLTVDKSRWQQGNVFSCSVMHEALHNHYTQKSLSLSPG |
| 83 | IgG1 Fc | DKTHTCPPCPAPEAAGGPSVFLFPPKPKDTLMISRTPEVTCVVVDV |
| | LALA PA | SHEDPEVKFNWYVDGVEVHNAKTKPREEQYNSTYRVVSVLTVLHQD |
| | LS without | WLNGKEYKCKVSNKALAAPIEKTISKAKGQPREPQVYTLPPSRDEL |
| | C-terminal | TKNQVSLTCLVKGFYPSDIAVEWESNGQPENNYKTTPPVLDSDGSF |
| | lysine | FLYSKLTVDKSRWQQGNVFSCSVLHEALHSHYTQKSLSLSPG |

In some embodiments, any IgG Fc, or derivative thereof, can be linked to the N-terminus or C-terminus of any of the embodiments described in Table 4 or 5 above with or without an IgG Fc linker. In some embodiments, human IgG1 Fc, or a derivative thereof, can be linked to the N-terminus or C-terminus of any of the embodiments described in Table 4 or 5 above with or without an IgG Fc linker. In some embodiments, the amino acid sequence of the human IgG1 Fc comprises or consists of the amino acid sequence of SEQ ID NO: 76 or 80. In some embodiments, the derivative if human IgG1 Fc comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or 99% identical to the amino acid sequence of SEQ ID NO: 76 or 80.

In some embodiments, a human IgG1 Fc comprising a LALA mutation, or a derivative thereof, can be linked to the N-terminus or C-terminus of any of the embodiments described in Table 4 or 5 above with or without an IgG Fc linker. In some embodiments, the amino acid sequence of the human IgG1 Fc comprising a LALA mutation comprises or consists of the amino acid sequence of SEQ ID NO: 77 or 81. In some embodiments, the derivative if human IgG1 Fc comprising a LALA mutation comprises an amino acid sequence at least 85, 90, 95, 96, 97, 98, or 99% identical to the amino acid sequence of SEQ ID NO: 77 or 81.

In some embodiments, a human IgG1 Fc comprising a LALA PA mutation, or a derivative thereof, can be linked to the N-terminus or C-terminus of any of the embodiments described in Table 4 or 5 above with or without an IgG Fc linker. In some embodiments, the amino acid sequence of the human IgG1 Fc comprising a LALA PA mutation comprises or consists of the amino acid sequence of SEQ ID NO: 78 or 82. In some embodiments, the derivative if human IgG1 Fc comprising a LALA PA mutation comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or 99% identical to the amino acid sequence of SEQ ID NO: 78 or 82.

In some embodiments, a human IgG1 Fc comprising a LALA PA LS mutation, or a derivative thereof, can be linked to the N-terminus or C-terminus of any of the embodiments described in Table 4 or 5 above with or without an IgG Fc linker. In some embodiments, the amino acid sequence of the human IgG1 Fc comprising a LALA PA LS mutation comprises or consists of the amino acid sequence of SEQ ID NO: 79 or 83. In some embodiments, the derivative if human IgG1 Fc comprising a LALA PA LS mutation comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or 99% identical to the amino acid sequence of SEQ ID NO: 79 or 83.

In some embodiments, the fusion protein comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or 99% identical to the amino acid sequences shown in Table 7. In some embodiments, the fusion protein comprises or consists of the amino acid sequences shown in Table 7.

TABLE 7
Fusion Protein Amino Acid Sequences

| SEQ | |
|---|---|
| ID | |
| NO: | Sequence |
| 84 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTASDAAGADANAGARQLYS |
| | ALANKCCHVGCTKRSLAQFC |
| 85 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTASDAAGANADAGARQLYS |
| | ALANKCCHVGCTKRSLAQFC |
| 86 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTASDAAGADADAGARQLYS |
| | ALANKCCHVGCTKRSLAQFC |
| 87 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTASDAAGAEAEAGARQLYS |
| | ALANKCCHVGCTKRSLAQFC |
| 88 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTASDAAGAQADAGARQLYS |
| | ALANKCCHVGCTKRSLAQFC |
| 89 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTASDAAGADAQAGARQLYS |
| | ALANKCCHVGCTKRSLAQFC |
| 90 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTASDAAGAQAQAGARQLYS |
| | ALANKCCHVGCTKRSLAQFC |
| 91 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGADANAGARQLYS |
| | ALANKCCHVGCTKRSLAQFC |
| 92 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGANADAGARQLYS |
| | ALANKCCHVGCTKRSLAQFC |
| 93 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGADADAGARQLYS |
| | ALANKCCHVGCTKRSLAQFC |
| 94 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGAEAEAGARQLYS |
| | ALANKCCHVGCTKRSLAQFC |
| 95 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGAQADAGARQLYS |
| | ALANKCCHVGCTKRSLAQFC |
| 96 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGADAQAGARQLYS |
| | ALANKCCHVGCTKRSLAQFC |
| 97 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGAQAQAGARQLYS |
| | ALANKCCHVGCTKRSLAQFC |
| 98 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGADANAGARQLYS |
| | ALANKCCHVGCTKRSLAQFC |
| 99 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGANADAGARQLYS |
| | ALANKCCHVGCTKRSLAQFC |
| 100 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGADADAGARQLYS |
| | ALANKCCHVGCTKRSLAQFC |
| 101 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGAEAEAGARQLYS |
| | ALANKCCHVGCTKRSLAQFC |
| 102 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGAQADAGARQLYS |
| | ALANKCCHVGCTKRSLAQFC |
| 103 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGADAQAGARQLYS |
| | ALANKCCHVGCTKRSLAQFC |
| 104 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGAQAQAGARQLYS |
| | ALANKCCHVGCTKRSLAQFC |
| 105 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGADANAGARQLYS |
| | ALANKCCHVGCTKQSLAQFC |
| 106 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGANADAGARQLYS |
| | ALANKCCHVGCTKQSLAQFC |
| 107 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGADADAGARQLYS |
| | ALANKCCHVGCTKQSLAQFC |
| 108 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGAEAEAGARQLYS |
| | ALANKCCHVGCTKQSLAQFC |
| 109 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGAQADAGARQLYS |
| | ALANKCCHVGCTKQSLAQFC |
| 110 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGADAQAGARQLYS |
| | ALANKCCHVGCTKQSLAQFC |
| 111 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGKSTASDAAGAQAQAGARQLYS |
| | ALANKCCHVGCTKQSLAQFC |
| 112 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGADANAGARQLYS |
| | ALANKCCHVGCTKQSLAQFC |
| 113 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGANADAGARQLYS |
| | ALANKCCHVGCTKQSLAQFC |
| 114 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGADADAGARQLYS |
| | ALANKCCHVGCTKQSLAQFC |
| 115 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGAEAEAGARQLYS |
| | ALANKCCHVGCTKQSLAQFC |
| 116 | DKTHTCPPCPAPEAAGGPSVFLFPPKPKDTLMISRTPEVTCVVVDVSHEDPEVKE |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGAQADAGARQLYS |
| | ALANKCCHVGCTKQSLAQFC |
| 117 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGADAQAGARQLYS |
| | ALANKCCHVGCTKQSLAQFC |
| 118 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSWQEEVIKLCGRELVRAQIAICGQSTASDAAGAQAQAGARQLYS |
| | ALANKCCHVGCTKQSLAQFC |
| 119 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTASDAAGAEAEAGARQLYS |
| | ALANKCCHVGCTKRSLAQFC |
| 120 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSFQEEVIKLCGRELVRAQIAICGKSTASDAAGAEAEAGARQLYS |
| | ALANKCCHVGCTKRSLAQFC |
| 121 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSLQEEVIKLCGRELVRAQIAICGKSTASDAAGAEAEAGARQLYS |
| | ALANKCCHVGCTKRSLAQFC |
| 122 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSIQEEVIKLCGRELVRAQIAICGKSTASDAAGAEAEAGARQLYS |
| | ALANKCCHVGCTKRSLAQFC |
| 123 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTASDAAGAEAEAGARQLYS |
| | ALANKCCYVGCTKRSLAQFC |
| 124 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTASDAAGAEAEAGARQLYS |
| | ALANKCCLVGCTKRSLAQFC |
| 125 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTASDAAGAEAEAGARQLYS |
| | ALANKCCQVGCTKRSLAQFC |
| 126 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTASDAAGAEAEAGARQLYS |
| | ALANKCCKVGCTKRSLAQFC |
| 127 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSYQEEVIKLCGRELVRAQIAICGKSTASDAAGAEAEAGARQLYS |
| | ALANKCCYVGCTKRSLAQFC |
| 128 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSYQEEVIKLCGRELVRAQIAICGKSTASDAAGAEAEAGARQLYS |
| | ALANKCCKVGCTKRSLAQFC |
| 129 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSYQEEVIKLCGRELVRAQIAICGKSTGGEGSGGEGEGGGRQLYS |
| | ALANKCCYVGCTKRSLAQFC |
| 130 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSYQEEVIKLCGRELVRAQIAICGKSTGGEGSGGEGEGGGRQLYS |
| | ALANKCCKVGCTKRSLAQFC |
| 131 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSYQEEVIKLCGRELVRAQIAICGKSTGGEGSGGEGSGGGRQLYS |
| | ALANKCCYVGCTKRSLAQFC |
| 132 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGEGGSDSYQEEVIKLCGRELVRAQIAICGKSTGGEGSGGEGSGGGRQLYS |
| | ALANKCCKVGCTKRSLAQFC |
| 133 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLIVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTASDAAGAEAEAGARQLYS |
| | ALANKCCYVGCTKQSLAQFC |
| 134 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTASDAAGAEAEAGARQLYS |
| | ALANKCCKVGCTKQSLAQFC |
| 135 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGEGGGRQLYS |
| | ALANKCCYVGCTKQSLAQFC |
| 136 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGEGGGRQLYS |
| | ALANKCCKVGCTKQSLAQFC |
| 137 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGSGGGRQLYS |
| | ALANKCCYVGCTKQSLAQFC |
| 138 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGSGGGRQLYS |
| | ALANKCCKVGCTKQSLAQFC |
| 300 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWKEEVIKLCGRELVRAQIAICGKSTGGGEGGGEGGGEGRQLYS |
| | ALANKCCHVGCTKRSLARFC |
| 301 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWKEEVIKLCGRELVRAQIAICGKSTGGGEGGGEGGGEGGGQLY |
| | SALANKCCHVGCTKRSLARFC |
| 302 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWMEEVIKLCGRELVRAQIAICGKSTGGGEGGGEGGGEGRQLYS |
| | ALANKCCHVGCTKRSLARFC |
| 303 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWQEEVIKLCGRELVRAQIAICGKSTGGGEGGGEGGGEGRQLYS |
| | ALANKCCHVGCTKRSLARFC |
| 304 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTGGGEGGGEGGGEGRQLYS |
| | ALANKCCHVGCTKRSLARFC |
| 305 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWMEEVIKLCGRELVRAQIAICGKSTGGGEGGGEGGGEGGGQLY |
| | SALANKCCHVGCTKRSLARFC |
| 306 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWQEEVIKLCGRELVRAQIAICGKSTGGGEGGGEGGGEGGGQLY |
| | SALANKCCHVGCTKRSLARFC |
| 307 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTGGGEGGGEGGGEGGGQLY |
| | SALANKCCHVGCTKRSLARFC |
| 308 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWMEEVIKLCGRELVRAQIAICGKSTGGEGGGEEGGGEGGQLYS |
| | ALANKCCHVGCTKRSLARFC |
| 309 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWQEEVIKLCGRELVRAQIAICGKSTGGEGGGEEGGGEGGQLYS |
| | ALANKCCHVGCTKRSLARFC |
| 310 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGGGSDSWMEEVIKLCGRELVRAQIAICGKSTGGEGGGEEGGGEGGQLYSA |
| | LANKCCHVGCTKRSLARFC |
| 311 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGGGSDSWQEEVIKLCGRELVRAQIAICGKSTGGEGGGEEGGGEGGQLYSA |
| | LANKCCHVGCTKRSLARFC |
| 312 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWQEEVIKLCGRELVRAQIAICGKSTGGGEGGGEGGGEGRQLYS |
| | ALANKCCHVGCTKRSLAQFC |
| 313 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTGGGEGGGEGGGEGRQLYS |
| | ALANKCCHVGCTKRSLAQFC |
| 314 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGGGSDSWQEEVIKLCGRELVRAQIAICGKSTGGGEGGGEGGGEGRQLYSA |
| | LANKCCHVGCTKRSLARFC |
| 315 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGGGSDSWQEEVIKLCGRELVRAQIAICGKSTGGGEGGGEGGGEGRQLYSA |
| | LANKCCHVGCTKRSLARFC |
| 316 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGGGSDSWQEEVIKLCGRELVRAQIAICGKSTGGGEGGGEGGGEGRQLYSA |
| | LANKCCHVGCTKRSLAQFC |
| 317 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGGGSDSWQEEVIKLCGRELVRAQIAICGKSTGGGEGGGEGGGEGRQLYSA |
| | LANKCCHVGCTKRSLAQFC |
| 318 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWQEEVIKLCGRELVRAQIAICGKSTGGGEEGGGEEGGGRQLYS |
| | ALANKCCHVGCTKRSLARFC |
| 319 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTGGGEEGGGEEGGGRQLYS |
| | ALANKCCHVGCTKRSLARFC |
| 320 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWQEEVIKLCGRELVRAQIAICGKSTGGGEEGGGEEGGGRQLYS |
| | ALANKCCHVGCTKRSLAQFC |
| 321 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTGGGEEGGGEEGGGRQLYS |
| | ALANKCCHVGCTKRSLAQFC |
| 322 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGGGSDSWQEEVIKLCGRELVRAQIAICGKSTGGGEEGGGEEGGGRQLYSA |
| | LANKCCHVGCTKRSLARFC |
| 323 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGGGSDSWQEEVIKLCGRELVRAQIAICGKSTGGGEEGGGEEGGGRQLYSA |
| | LANKCCHVGCTKRSLARFC |
| 324 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGGGSDSWQEEVIKLCGRELVRAQIAICGKSTGGGEEGGGEEGGGRQLYSA |
| | LANKCCHVGCTKRSLAQFC |
| 325 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGGGSDSWQEEVIKLCGRELVRAQIAICGKSTGGGEEGGGEEGGGRQLYSA |
| | LANKCCHVGCTKRSLAQFC |
| 326 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTGGEGEGGEGEGGSRQLYS |
| | ALANKCCHVGCTKRSLAQFC |
| 327 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGGGSDSWQEEVIKLCGRELVRAQIAICGKSTGGEGEGGEGEGGSRQLYSA |
| | LANKCCHVGCTKRSLARFC |
| 328 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGGGSDSWQEEVIKLCGRELVRAQIAICGKSTGGEGEGGEGEGGSRQLYSA |
| | LANKCCHVGCTKRSLAQFC |
| 359 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWKEEVIKLCGRELVRAQIAICGKSTASDAAGANANAGARQLYS |
| | ALANKCCHVGCTKRSLAQFC |
| 360 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGKGGSDSWKEEVIKLCGRELVRAQIAICGKSTASDAAGANANAGARQLYS |
| | ALANKCCHVGCTKRSLAEFC |
| 361 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWKEEVIKLCGRELVRAQIAICGKSTGGGEGGGEGGGEGRQLYS |
| | ALANKCCHVGCTKRSLARFC |
| 362 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWKEEVIKLCGRELVRAQIAICGKSTGGGEGGGEGGGEGGGQLY |
| | SALANKCCHVGCTKRSLARFC |
| 363 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWMEEVIKLCGRELVRAQIAICGKSTGGGEGGGEGGGEGRQLYS |
| | ALANKCCHVGCTKRSLARFC |
| 364 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWMEEVIKLCGRELVRAQIAICGKSTGGGEGGGEGGGEGGGQLY |
| | SALANKCCHVGCTKRSLARFC |
| 365 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWMEEVIKLCGRELVRAQIAICGKSTGGEGGGEEGGGEGGQLYS |
| | ALANKCCHVGCTKRSLARFC |
| 366 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWQEEVIKLCGRELVRAQIAICGKSTGGEGGGEEGGGEGGQLYS |
| | ALANKCCHVGCTKRSLARFC |
| 367 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGGGSDSWMEEVIKLCGRELVRAQIAICGKSTGGEGGGEEGGGEGGQLYSA |
| | LANKCCHVGCTKRSLARFC |
| 368 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGGGSDSWQEEVIKLCGRELVRAQIAICGKSTGGEGGGEEGGGEGGQLYSA |
| | LANKCCHVGCTKRSLARFC |
| 369 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWKEEVIKLCGRELVRAQIAICGKSTASDAAGANANAGARQLYS |
| | ALANKCCHVGCTKRSLAQFC |
| 370 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSWKEEVIKLCGRELVRAQIAICGKSTASDAAGANANAGARQLYS |
| | ALANKCCHVGCTKRSLAEFC |
| 516 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGEGGGRQLYS |
| | ALANKCCHVGCTKRSLAQFC |
| 517 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGEGGGRQLYS |
| | ALANKCCQVGCTKRSLAQFC |
| 518 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGEGGGRQLYS |
| | ALANKCCHVGCTKQSLAQFC |
| 519 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGEGGGRQLYS |
| | ALANKCCQVGCTKQSLAQFC |
| 520 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGSGGGRQLYS |
| | ALANKCCHVGCTKRSLAQFC |
| 521 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGSGGGRQLYS |
| | ALANKCCQVGCTKRSLAQFC |
| 522 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGSGGGRQLYS |
| | ALANKCCHVGCTKQSLAQFC |
| 523 | DKTHTCPPCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALAA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVLHEALHSHYTQKS |
| | LSLSPGKGGSDSYQEEVIKLCGRELVRAQIAICGKSTGGEGSGGEGSGGGRQLYS |
| | ALANKCCQVGCTKQSLAQFC |

In some embodiments, the IgG Fc comprises a mouse IgG kappa signal sequence comprising the amino acid sequence of METDTLLLWVLLLWVPGSTG (SEQ ID NO: 329). In some embodiments, the IgG Fc comprises a mouse IgG heavy chain signal sequence. In some embodiments, the IgG Fc comprises a signal sequence comprising the amino acid sequence of MGWSCIILFLVATATGVHS (SEQ ID NO: 548). In some embodiments a different signal sequence is used. In some embodiments, no signal sequence is present on the fusion protein as produced.

In some embodiments, the fusion protein comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or 99% identical to the amino acid sequences shown in Table 8. In some embodiments, the fusion protein comprises or consists of the amino acid sequences shown in Table 8.

TABLE 8
Fusion Protein Amino Acid Sequences

| SEQ | |
|---|---|
| ID | |
| NO: | Sequence |
| 139 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STASDAAGADANAGARQLYSALANKCCHVGCTKRSLAQFC |
| 140 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STASDAAGANADAGARQLYSALANKCCHVGCTKRSLAQFC |
| 141 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STASDAAGADADAGARQLYSALANKCCHVGCTKRSLAQFC |
| 142 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STASDAAGAEAEAGARQLYSALANKCCHVGCTKRSLAQFC |
| 143 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STASDAAGAQADAGARQLYSALANKCCHVGCTKRSLAQFC |
| 144 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STASDAAGADAQAGARQLYSALANKCCHVGCTKRSLAQFC |
| 145 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STASDAAGAQAQAGARQLYSALANKCCHVGCTKRSLAQFC |
| 146 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGADANAGARQLYSALANKCCHVGCTKRSLAQFC |
| 147 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGANADAGARQLYSALANKCCHVGCTKRSLAQFC |
| 148 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGADADAGARQLYSALANKCCHVGCTKRSLAQFC |
| 149 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGAEAEAGARQLYSALANKCCHVGCTKRSLAQFC |
| 150 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGAQADAGARQLYSALANKCCHVGCTKRSLAQFC |
| 151 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGADAQAGARQLYSALANKCCHVGCTKRSLAQFC |
| 152 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGAQAQAGARQLYSALANKCCHVGCTKRSLAQFC |
| 153 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGADANAGARQLYSALANKCCHVGCTKRSLAQFC |
| 154 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGANADAGARQLYSALANKCCHVGCTKRSLAQFC |
| 155 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGADADAGARQLYSALANKCCHVGCTKRSLAQFC |
| 156 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGAEAEAGARQLYSALANKCCHVGCTKRSLAQFC |
| 157 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGAQADAGARQLYSALANKCCHVGCTKRSLAQFC |
| 158 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGADAQAGARQLYSALANKCCHVGCTKRSLAQFC |
| 159 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGAQAQAGARQLYSALANKCCHVGCTKRSLAQFC |
| 160 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGADANAGARQLYSALANKCCHVGCTKQSLAQFC |
| 161 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGANADAGARQLYSALANKCCHVGCTKQSLAQFC |
| 162 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGADADAGARQLYSALANKCCHVGCTKQSLAQFC |
| 163 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGAEAEAGARQLYSALANKCCHVGCTKQSLAQFC |
| 164 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGAQADAGARQLYSALANKCCHVGCTKQSLAQFC |
| 165 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGADAQAGARQLYSALANKCCHVGCTKQSLAQFC |
| 166 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGK |
| | STASDAAGAQAQAGARQLYSALANKCCHVGCTKQSLAQFC |
| 167 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGADANAGARQLYSALANKCCHVGCTKQSLAQFC |
| 168 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGANADAGARQLYSALANKCCHVGCTKQSLAQFC |
| 169 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGADADAGARQLYSALANKCCHVGCTKQSLAQFC |
| 170 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGAEAEAGARQLYSALANKCCHVGCTKQSLAQFC |
| 171 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGAQADAGARQLYSALANKCCHVGCTKQSLAQFC |
| 172 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGADAQAGARQLYSALANKCCHVGCTKQSLAQFC |
| 173 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSWQEEVIKLCGRELVRAQIAICGQ |
| | STASDAAGAQAQAGARQLYSALANKCCHVGCTKQSLAQFC |
| 174 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STASDAAGAEAEAGARQLYSALANKCCHVGCTKRSLAQFC |
| 175 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSFQEEVIKLCGRELVRAQIAICGK |
| | STASDAAGAEAEAGARQLYSALANKCCHVGCTKRSLAQFC |
| 176 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSLQEEVIKLCGRELVRAQIAICGK |
| | STASDAAGAEAEAGARQLYSALANKCCHVGCTKRSLAQFC |
| 177 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSIQEEVIKLCGRELVRAQIAICGK |
| | STASDAAGAEAEAGARQLYSALANKCCHVGCTKRSLAQFC |
| 178 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STASDAAGAEAEAGARQLYSALANKCCYVGCTKRSLAQFC |
| 179 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STASDAAGAEAEAGARQLYSALANKCCLVGCTKRSLAQFC |
| 180 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STASDAAGAEAEAGARQLYSALANKCCQVGCTKRSLAQFC |
| 181 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STASDAAGAEAEAGARQLYSALANKCCKVGCTKRSLAQFC |
| 182 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSYQEEVIKLCGRELVRAQIAICGK |
| | STASDAAGAEAEAGARQLYSALANKCCYVGCTKRSLAQFC |
| 183 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSYQEEVIKLCGRELVRAQIAICGK |
| | STASDAAGAEAEAGARQLYSALANKCCKVGCTKRSLAQFC |
| 184 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSYQEEVIKLCGRELVRAQIAICGK |
| | STGGEGSGGEGEGGGRQLYSALANKCCYVGCTKRSLAQFC |
| 185 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSYQEEVIKLCGRELVRAQIAICGK |
| | STGGEGSGGEGEGGGRQLYSALANKCCKVGCTKRSLAQFC |
| 186 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSYQEEVIKLCGRELVRAQIAICGK |
| | STGGEGSGGEGSGGGRQLYSALANKCCYVGCTKRSLAQFC |
| 187 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGEGGSDSYQEEVIKLCGRELVRAQIAICGK |
| | STGGEGSGGEGSGGGRQLYSALANKCCKVGCTKRSLAQFC |
| 188 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STASDAAGAEAEAGARQLYSALANKCCYVGCTKQSLAQFC |
| 189 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STASDAAGAEAEAGARQLYSALANKCCKVGCTKQSLAQFC |
| 190 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGEGGGRQLYSALANKCCYVGCTKQSLAQFC |
| 191 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGEGGGRQLYSALANKCCKVGCTKQSLAQFC |
| 192 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGSGGGRQLYSALANKCCYVGCTKQSLAQFC |
| 193 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGSGGGRQLYSALANKCCKVGCTKQSLAQFC |
| 330 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGSDSWKEEVIKLCGRELVRAQIAICGK |
| | STGGGEGGGEGGGEGRQLYSALANKCCHVGCTKRSLARFC |
| 331 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGSDSWKEEVIKLCGRELVRAQIAICGK |
| | STGGGEGGGEGGGEGGGQLYSALANKCCHVGCTKRSLARFC |
| 332 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGSDSWMEEVIKLCGRELVRAQIAICGK |
| | STGGGEGGGEGGGEGRQLYSALANKCCHVGCTKRSLARFC |
| 333 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGSDSWQEEVIKLCGRELVRAQIAICGK |
| | STGGGEGGGEGGGEGRQLYSALANKCCHVGCTKRSLARFC |
| 334 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STGGGEGGGEGGGEGRQLYSALANKCCHVGCTKRSLARFC |
| 335 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGSDSWMEEVIKLCGRELVRAQIAICGK |
| | STGGGEGGGEGGGEGGGQLYSALANKCCHVGCTKRSLARFC |
| 336 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGSDSWQEEVIKLCGRELVRAQIAICGK |
| | STGGGEGGGEGGGEGGGQLYSALANKCCHVGCTKRSLARFC |
| 337 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STGGGEGGGEGGGEGGGQLYSALANKCCHVGCTKRSLARFC |
| 338 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGSDSWMEEVIKLCGRELVRAQIAICGK |
| | STGGEGGGEEGGGEGGQLYSALANKCCHVGCTKRSLARFC |
| 339 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGSDSWQEEVIKLCGRELVRAQIAICGK |
| | STGGEGGGEEGGGEGGQLYSALANKCCHVGCTKRSLARFC |
| 340 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGGGSDSWMEEVIKLCGRELVRAQIAICGKS |
| | TGGEGGGEEGGGEGGQLYSALANKCCHVGCTKRSLARFC |
| 341 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGGGSDSWQEEVIKLCGRELVRAQIAICGKS |
| | TGGEGGGEEGGGEGGQLYSALANKCCHVGCTKRSLARFC |
| 342 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGSDSWQEEVIKLCGRELVRAQIAICGK |
| | STGGGEGGGEGGGEGRQLYSALANKCCHVGCTKRSLAQFC |
| 343 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STGGGEGGGEGGGEGRQLYSALANKCCHVGCTKRSLAQFC |
| 344 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGGGSDSWQEEVIKLCGRELVRAQIAICGKS |
| | TGGGEGGGEGGGEGRQLYSALANKCCHVGCTKRSLARFC |
| 345 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGGGSDSWQEEVIKLCGRELVRAQIAICGKS |
| | TGGGEGGGEGGGEGRQLYSALANKCCHVGCTKRSLARFC |
| 346 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGGGSDSWQEEVIKLCGRELVRAQIAICGKS |
| | TGGGEGGGEGGGEGRQLYSALANKCCHVGCTKRSLAQFC |
| 347 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGGGSDSWQEEVIKLCGRELVRAQIAICGKS |
| | TGGGEGGGEGGGEGRQLYSALANKCCHVGCTKRSLAQFC |
| 348 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGSDSWQEEVIKLCGRELVRAQIAICGK |
| | STGGGEEGGGEEGGGRQLYSALANKCCHVGCTKRSLARFC |
| 349 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STGGGEEGGGEEGGGRQLYSALANKCCHVGCTKRSLARFC |
| 350 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGSDSWQEEVIKLCGRELVRAQIAICGK |
| | STGGGEEGGGEEGGGRQLYSALANKCCHVGCTKRSLAQFC |
| 351 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STGGGEEGGGEEGGGRQLYSALANKCCHVGCTKRSLAQFC |
| 352 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGGGSDSWQEEVIKLCGRELVRAQIAICGKS |
| | TGGGEEGGGEEGGGRQLYSALANKCCHVGCTKRSLARFC |
| 353 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGGGSDSWQEEVIKLCGRELVRAQIAICGKS |
| | TGGGEEGGGEEGGGRQLYSALANKCCHVGCTKRSLARFC |
| 354 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGGGSDSWQEEVIKLCGRELVRAQIAICGKS |
| | TGGGEEGGGEEGGGRQLYSALANKCCHVGCTKRSLAQFC |
| 355 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGGGSDSWQEEVIKLCGRELVRAQIAICGKS |
| | TGGGEEGGGEEGGGRQLYSALANKCCHVGCTKRSLAQFC |
| 356 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STGGEGEGGEGEGGSRQLYSALANKCCHVGCTKRSLAQFC |
| 357 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGGGSDSWQEEVIKLCGRELVRAQIAICGKS |
| | TGGEGEGGEGEGGSRQLYSALANKCCHVGCTKRSLARFC |
| 358 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGGGSDSWQEEVIKLCGRELVRAQIAICGKS |
| | TGGEGEGGEGEGGSRQLYSALANKCCHVGCTKRSLAQFC |
| 371 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWKEEVIKLCGRELVRAQIAICGK |
| | STGGGEGGGEGGGEGRQLYSALANKCCHVGCTKRSLARFC |
| 372 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWKEEVIKLCGRELVRAQIAICGK |
| | STGGGEGGGEGGGEGGGQLYSALANKCCHVGCTKRSLARFC |
| 373 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWMEEVIKLCGRELVRAQIAICGK |
| | STGGGEGGGEGGGEGRQLYSALANKCCHVGCTKRSLARFC |
| 374 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWMEEVIKLCGRELVRAQIAICGK |
| | STGGGEGGGEGGGEGGGQLYSALANKCCHVGCTKRSLARFC |
| 375 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWMEEVIKLCGRELVRAQIAICGK |
| | STGGEGGGEEGGGEGGQLYSALANKCCHVGCTKRSLARFC |
| 376 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWQEEVIKLCGRELVRAQIAICGK |
| | STGGEGGGEEGGGEGGQLYSALANKCCHVGCTKRSLARFC |
| 377 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGGGSDSWMEEVIKLCGRELVRAQIAICGKS |
| | TGGEGGGEEGGGEGGQLYSALANKCCHVGCTKRSLARFC |
| 378 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGGGSDSWQEEVIKLCGRELVRAQIAICGKS |
| | TGGEGGGEEGGGEGGQLYSALANKCCHVGCTKRSLARFC |
| 379 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWKEEVIKLCGRELVRAQIAICGK |
| | STASDAAGANANAGARQLYSALANKCCHVGCTKRSLAQFC |
| 380 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSWKEEVIKLCGRELVRAQIAICGK |
| | STASDAAGANANAGARQLYSALANKCCHVGCTKRSLAEFC |
| 498 | METDTLLLWVLLLWVPGSTGDKTHTCPPCPAPELLGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVMHEALHNHYTQKSLSLSPGKGGGGSGGGGSGGGGSQLYSALANKCCHV |
| | GCTKRSLARFCGGGGSGGGGSGGGGSSWMEEVIKLCGRELVRAQIAICGMSTWS |
| 524 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGEGGGRQLYSALANKCCHVGCTKRSLAQFC |
| 525 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGEGGGRQLYSALANKCCQVGCTKRSLAQFC |
| 526 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGEGGGRQLYSALANKCCHVGCTKQSLAQFC |
| 527 | METDTLLLWVLLLWVPGSTGDKTHTCPPCPAPEAAGGPSVFLFPPKPKDTLMISR |
| | TPEVTCVVVDVSHEDPVEKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGEGGGRQLYSALANKCCQVGCTKQSLAQFC |
| 528 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGSGGGRQLYSALANKCCHVGCTKRSLAQFC |
| 529 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGSGGGRQLYSALANKCCQVGCTKRSLAQFC |
| 530 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGSGGGRQLYSALANKCCHVGCTKQSLAQFC |
| 531 | METDTLLLWVLLLWVPGSTGDKTHTCPPCPAPEAAGGPSVFLFPPKPKDTLMISR |
| | TPEVTCVVVDVSHEDPEVKFNWYVDGVEVHNAKTKPREEQYNSTYRVVSVLTVLH |
| | QDWLNGKEYKCKVSNKALAAPIEKTISKAKGQPREPQVYTLPPSRDELTKNQVSL |
| | TCLVKGFYPSDIAVEWESNGQPENNYKTTPPVLDSDGSFFLYSKLTVDKSRWQQG |
| | NVFSCSVLHEALHSHYTQKSLSLSPGKGGSDSYQEEVIKLCGRELVRAQIAICGK |
| | STGGEGSGGEGSGGGRQLYSALANKCCQVGCTKQSLAQFC |
| 549 | MGWSCIILFLVATATGVHSDKTHTCPPCPAPEAAGGPSVFLFPPKPKDTLMISRT |
| | PEVTCVVVDVSHEDPVEKFNWYVDGVEVHNAKTKPREEQYNSTYRVVSVLTVLHQ |
| | DWLNGKEYKCKVSNKALAAPIEKTISKAKGQPREPQVYTLPPSRDELTKNQVSLT |
| | CLVKGFYPSDIAVEWESNGQPENNYKTTPPVLDSDGSFFLYSKLTVDKSRWQQGN |
| | VFSCSVLHEALHSHYTQKSLSLSPGKGGSDSWQEEVIKLCGRELVRAQIAICGKS |
| | TASDAAGAEAEAGARQLYSALANKCCHVGCTKRSLAQFC |

### Other Half-Life Extending Moieties

As used herein, the term “half-life extending moiety” includes non-proteinaceous, half-life extending moieties, such as PEG or HES, and proteinaceous half-life extending moieties such as Fc domain. In some embodiments, non-proteinaceous half-life extending moieties are linked to the fusion proteins described herein. In some embodiments, the non-proteinaceous half-life extending moieties are linked to the fusion proteins instead of IgG Fc. In some embodiments, the non-proteinaceous half-life extending moieties are linked to the fusion proteins in addition to IgG Fc.

Examples of suitable polymer molecules that act as non-proteinaceous half-life extending moieties include polymer molecules selected from the group consisting of polyalkylene oxide (PAO), including polyalkylene glycol (PAG), such as polyethylene glycol (PEG) and polypropylene glycol (PPG), branched PEGs, hydroxyalkyl starch (HAS), such as hydroxyethyl starch (HES), polysialic acid (PSA), poly-vinyl alcohol (PVA), poly-carboxylate, poly-(vinylpyrrolidone), polyethylene-co-maleic acid anhydride, polystyrene-co-maleic acid anhydride, dextran, including carboxymethyl-dextran, or any other biopolymer suitable for reducing immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG has the advantage of having only few reactive groups capable of cross-linking compared to, e.g., polysaccharides such as dextran. In particular, monofunctional PEG, e.g., methoxypolyethylene glycol (mPEG), is of interest since its coupling chemistry is relatively simple (only one reactive group is available for conjugating with attachment groups on the polypeptide). Consequently, as the risk of cross-linking is eliminated, the resulting conjugated fusion proteins described herein are more homogeneous, and the reaction of the polymer molecules with the variant polypeptide is easier to control.

To effect covalent attachment of the polymer molecule(s) to the fusion proteins described herein, the hydroxyl end groups of the polymer molecule must be provided in activated form, i.e., with reactive functional groups (examples of which include primary amino groups, hydrazide (HZ), thiol, succinate (SUC), succinimidyl succinate (SS), succinimidyl succinamide (SSA), succinimidyl propionate (SPA), succinimidyl butyrate (SBA), succinimidyl carboxymethylate (SCM), benzotriazole carbonate (BTC), N-hydroxysuccinimide (NHS), aldehyde, nitrophenylcarbonate (NPC), and tresylate (TRES)). Suitable activated polymer molecules are commercially available, e.g., from Shearwater Polymers, Inc., Huntsville, Ala., USA, or from PolyMASC Pharmaceuticals plc, UK.

Alternatively, the polymer molecules can be activated by conventional methods known in the art, e.g., as disclosed in WO 90/13540. Specific examples of activated linear or branched polymer molecules for use herein are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g., SPA-PEG, SSPA-PEG, SBA-PEG, SS-PEG, SSA-PEG, SC-PEG, SG-PEG, and SCM-PEG), and NOR-PEG, BTC-PEG, EPOXPEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. Nos. 5,932,462 and 5,643,575, both of which are incorporated herein by reference. Furthermore, the following publications disclose useful polymer molecules and/or PEGylation chemistries: U.S. Pat. Nos. 5,824,778, 5,476,653, WO 97/32607, EP 229,108, EP 402,378, U.S. Pat. Nos. 4,902,502, 5,281,698, 5,122,614, 5,219,564, WO 92/16555, WO 94/04193, WO 94/14758, WO 94/17039, WO 94/18247, WO 94/28024, WO 95/00162, WO 95/11924, WO 95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. Nos. 5,473,034, 5,516,673, EP 605 963, U.S. Pat. No. 5,382,657, EP 510 356, EP 400 472, EP 183 503, and EP 154 316.

Specific examples of activated PEG polymers particularly preferred for coupling to cysteine residues, include the following linear PEGs: vinylsulfone-PEG (VS-PEG), preferably vinylsulfone-mPEG (VS-mPEG); maleimide-PEG (MAL-PEG), preferably maleimide-mPEG (MAL-mPEG) and orthopyridyl-disulfide-PEG (OPSS-PEG), preferably orthopyridyl-disulfide-mPEG (OPSS-mPEG). Typically, such PEG or mPEG polymers will have a size of about 5 kDa, about 10 kDa, about 12 kDa or about 20 kDa.

The conjugation of the fusion proteins described herein and the activated polymer molecules is conducted by use of any conventional method, e.g., as described in the following references (which also describe suitable methods for activation of polymer molecules): Harris and Zalipsky, eds., Poly(ethylene glycol) Chemistry and Biological Applications, AZC Washington; R. F. Taylor, (1991), “Protein immobilisation. Fundamental and applications,” Marcel Dekker, N.Y.; S. S. Wong, (1992), “Chemistry of Protein Conjugation and Crosslinking,” CRC Press, Boca Raton; G. T. Hermanson et al., (1993), “Immobilized Affinity Ligand Techniques”, Academic Press, N.Y.

The skilled person will be aware that the activation method and/or conjugation chemistry to be used depends on the attachment group(s) of the fusion protein (examples of which are given further above), as well as the functional groups of the polymer (e.g., being amine, hydroxyl, carboxyl, aldehyde, sulfhydryl, succinimidyl, maleimide, vinylsulfone or haloacetate). The PEGylation may be directed towards conjugation to all available attachment groups on the fusion protein (i.e., such attachment groups that are exposed at the surface of the polypeptide) or may be directed towards one or more specific attachment groups, e.g., the N-terminal amino group as described in U.S. Pat. No. 5,985,265 or to cysteine residues. Furthermore, the conjugation may be achieved in one step or in a stepwise manner (e.g., as described in WO 99/55377).

For PEGylation to cysteine residues (see above) the fusion protein is usually treated with a reducing agent, such as dithiothreitol (DDT) prior to PEGylation. The reducing agent is subsequently removed by any conventional method, such as by desalting. Conjugation of PEG to a cysteine residue typically takes place in a suitable buffer at pH 6-9 at temperatures varying from 4° C. to 25° C. for periods up to 16 hours.

It will be understood that the PEGylation is designed so as to produce the optimal molecule with respect to the number of PEG molecules attached, the size and form of such molecules (e.g., whether they are linear or branched), and the attachment site(s) in the fusion protein. The molecular weight of the polymer to be used may e.g., be chosen on the basis of the desired effect to be achieved.

In connection with conjugation to only a single attachment group on the fusion protein (e.g., the N-terminal amino group), it may be advantageous that the polymer molecule, which may be linear or branched, has a high molecular weight, preferably about 10-25 kDa, such as about 15-25 kDa, e.g., about 20 kDa.

Normally, the polymer conjugation is performed under conditions aimed at reacting as many of the available polymer attachment groups with polymer molecules. This is achieved by means of a suitable molar excess of the polymer relative to the polypeptide. Typically, the molar ratios of activated polymer molecules to polypeptide are up to about 1000-1, such as up to about 200-1, or up to about 100-1. In some cases, the ratio may be somewhat lower, however, such as up to about 50-1, 10-1, 5-1, 2-1 or 1-1 in order to obtain optimal reaction.

It is also contemplated to couple the polymer molecules to the fusion protein through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378).

Subsequent to the conjugation, residual activated polymer molecules are blocked according to methods known in the art, e.g., by addition of primary amine to the reaction mixture, and the resulting inactivated polymer molecules are removed by a suitable method.

It will be understood that depending on the circumstances, e.g., the amino acid sequence of the fusion protein, the nature of the activated PEG compound being used and the specific PEGylation conditions, including the molar ratio of PEG to polypeptide, varying degrees of PEGylation may be obtained, with a higher degree of PEGylation generally being obtained with a higher ratio of PEG to fusion protein. The PEGylated fusion proteins resulting from any given PEGylation process will, however, normally comprise a stochastic distribution of conjugated fusion protein having slightly different degrees of PEGylation.

For improvement of the biological half-life of the fusion proteins described herein, chemical modification such as PEGylation, or HESylation are applicable.

HAS and HES non-proteinaceous polymers, as well as methods of producing HAS or HES conjugates are disclosed for example in WO 02/080979, WO 03/070772, WO 057092391 and WO 057092390.

Polysialytion is another technology, which uses the natural polymer polysialic acid (PSA) to prolong the half-life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the fusion protein in the circulation and prevents it from being recognized by the immune system. The PSA polymer is naturally found in the human body.

It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

### Biological Activity of the Relaxin-2 Fusion Proteins

In some embodiments, the relaxin-2 fusion proteins described herein have high levels of biological activity as compared to native relaxin-2. In some embodiments, any of the relaxin-2 fusion proteins described herein have from about 1% to about 200% of a biological activity as compared to native relaxin-2. In some embodiments, the relaxin-2 fusion protein has at least about 5%, about 10%, about 15%, about 20%, about 25%, about 30%, about 35%, about 40%, about 50%, about 60%, about 70%, about 80%, about 90%, about 100%, about 125% about 150%, about 175%, or about 200% of a biological activity as compared to native relaxin-2.

In some embodiments, any of the relaxin-2 fusion proteins described herein have from about 1% to about 200% of maximal biological activity as compared to native relaxin-2. In some embodiments, maximal biological activity is the maximum response (Emax) of relaxin-2 or relaxin-2 fusion protein. In some embodiments, the relaxin-2 fusion protein has at least about 5%, about 10%, about 15%, about 20%, about 25%, about 30%, about 35%, about 40%, about 50%, about 60%, about 70%, about 80%, about 90%, about 100%, about 125% about 150%, about 175%, or about 200% of a maximal biological activity as compared to native relaxin-2.

In some embodiments, any of the relaxin-2 fusion proteins described herein have about at least about 0.001-fold to about at least 1,000-fold enhanced potency as compared to native relaxin-2. In some embodiments, potency is the concentration of relaxin-2 or relaxin-2 fusion protein to elicit a half-maximal response (EC₅₀). In some embodiments, the relaxin-2 fusion protein has at least about 0.001-fold, about 0.01-fold, about 0.1-fold, about 1-fold, about 10-fold, about 100-fold, or about 1,000-fold of the potency as compared to native relaxin-2.

The biological activity can be any biological activity of native relaxin-2. For example, the biological activity can be the capacity to bind the receptor of native relaxin-2, RXFP1. The binding of relaxin-2 to RXFP1 can be measured by any well-known methods in the art, such as radioligand binding. In some embodiments, the fusion proteins described herein bind to RXFP1 when it is expressed on a cell surface.

In some embodiments, the biological activity can be the capacity to activate RXFP1 on a cell surface. The activation of RXFP1 by the relaxin-2 fusion proteins described herein can be determined by the increase of cAMP using any methods well known in the art, such as measuring the activity of a cAMP-driven reporter gene, e.g., β-galactosidase. The activation of RXFP1 by the relaxin-2 fusion proteins described herein in a cell may also be determined by using a biosensor such as the GloSensor biosensor. The activation of RXFP1 by the relaxin-2 fusion proteins described herein in a cell may also be determined by measuring the expression of certain genes, such as angiogenic factors, e.g., VEGF, or the expression of MMPs using well-known methods in the art. In some embodiments, the biological activity is a physiological, biochemical activity or any other effect-inducing activity of the relaxin-2. Exemplary biological activities include, but are not limited to, vasodilation, collagen degradation, angiogenesis, decreasing arterial blood pressure, increasing renal artery blood flow, increasing renal plasma flow, increasing cardiac filling at diastole, resolving established fibrosis, and suppressing new fibrosis development.

In some embodiments, the fusion proteins described herein have improved pharmacokinetics profiles. Without wishing to be bound by any theory, the structure of the fusion proteins described herein is based upon, at least in part, the surprising discovery that reducing the pI of relaxin-2 fusion protein analogs increases their circulating half-life. In some embodiments, the fusion proteins described herein have high bioavailability. In some embodiments, the fusion proteins described herein have high and/or stable serum levels. In some embodiments, the circulating half-life, bioavailability, high serum level, and/or stable serum level is in a mammal. In some embodiments, the mammal is a rodent or a primate. In some embodiments, the rodent is a rat or a mouse. In some embodiments, the primate is a human or a monkey. In some embodiments, the monkey is a cynomolgus monkey. In some embodiments, the mammal is a human.

In some embodiments, the fusion proteins described herein may have a circulating half-life of greater than about 5 hours, 10 hours, 20 hours, 50 hours, 75 hours, 100 hours, 125 hours, 150 hours, or more. In some embodiments, the fusion proteins described herein may have a circulating half-life of 5-10 hours, 10-20 hours, 20-50 hours, 50-75 hours, 75-100 hours, 100-125 hours, or 125-150 hours. In some embodiments, the fusion proteins described herein may have a circulating half-life of about 10 days, about 11 days, about 12 days, about 13 days, about 14 days, about 15 days, about 16 days, about 17 days, about 18 days, about 19 days, about 20 days, about 21 days, about 22 days, or about 23 days. In some embodiments, the fusion proteins described herein may have a circulating half-life of 10 days, 11 days, 12 days, 13 days, 14 days, 15 days, 16 days, 17 days, 18 days, 19 days, 20 days, 21 days, 22 days, or 23 days. In some embodiments, the fusion proteins described herein may have a circulating half-life of at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, or at least 23 days. In some embodiments, the fusion proteins described herein may have a circulating half-life of greater than about 5 hours, 10 hours, 20 hours, 50 hours, 75 hours, 100 hours, 125 hours, 150 hours, or more, when administered to a human. In some embodiments, the fusion proteins described herein may have a circulating half-life of 5-10 hours, 10-20 hours, 20-50 hours, 50-75 hours, 75-100 hours, 100-125 hours, or 125-150 hours, when administered to a human. In some embodiments, the fusion proteins described herein may have a circulating half-life of about 10 days, about 11 days, about 12 days, about 13 days, about 14 days, about 15 days, about 16 days, about 17 days, about 18 days, about 19 days, about 20 days, about 21 days, about 22 days, or about 23 days, when administered to a human. In some embodiments, the fusion proteins described herein may have a circulating half-life of 10 days, 11 days, 12 days, 13 days, 14 days, 15 days, 16 days, 17 days, 18 days, 19 days, 20 days, 21 days, 22 days, or 23 days, when administered to a human. In some embodiments, the fusion proteins described herein may have a circulating half-life of at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, or at least 23 days, when administered to a human. Values and ranges intermediate to the recited values are also intended to be part of this disclosure. In some embodiments, the fusion proteins described herein have a longer circulating half-life than a native two chain relaxin-2. For example, the circulating half-life of a native two chain relaxin-2 may be less than about 5 hours. (See, e.g., Chen et al., The Pharmacokinetics of Recombinant Human Relaxin in Non-Pregnant Women after Intravenous, Intravaginal, and Intracervical Administration, Pharm. Res. 10:834038 (1993), incorporated herein by reference).

This increased half-life can be, at least in part, attributed to the reduced pI of the fusion proteins described herein. In some embodiments, the fusion protein has a pI that is less than about 9.4. As used herein, the term “about” when referring to pI encompasses variations of ±1% of a given value or range, as is appropriate to perform the methods disclosed herein. In some embodiments, the fusion protein has a pI that is less than 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, or 6.1, or is less than about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, or 6.1. In some embodiments, the fusion protein has a pI that is less than 9.0. In some embodiments, the fusion protein has a pI that is less than about 8.2. In some embodiments, the fusion protein has a pI from about 6.0 to about 9.4. In some embodiments, the fusion protein has a pI from about 6.5 to about 8.5, about 6.6 to about 8.4, about 6.7 to about 8.3, about 6.8 to about 8.2, about 6.8 to about 8.1, about 6.8 to about 8.0, about 6.8 to about 7.9, about 6.0 to about 8.2, about 6.0 to about 8.1, about 6.0 to about 8.0, about 6.0 to about 7.9, about 6.0 to about 7.8, about 6.0 to about 7.7, about 6.0 to about 7.6, about 6.0 to about 7.5, about 6.0 to about 7.4, about 6.0 to about 7.3, about 6.0 to about 7.2, about 6.0 to about 7.1, about 6.0 to about 7.0, about 6.0 to about 6.9, about 6.0 to about 6.8, about 6.0 to about 6.7, about 6.0 to about 6.6, about 6.0 to about 6.5, about 6.0 to about 6.4, about 6.0 to about 6.3, about 6.0 to about 6.2, or about 6.0 to about 6.1. In some embodiments, the fusion protein has a pI from about 6.0 to about 8.2. In some embodiments, the fusion protein has a pI of 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, or 8.2, or is about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, or 8.2, or is between any two such values. In some embodiments, the fusion protein has a pI of about 6.8. In some embodiments, the fusion protein has a pI of about 7.0. In some embodiments, the fusion protein has a pI of about 7.1. In some embodiments, the fusion protein has a pI of about 7.4. In some embodiments, the fusion protein has a pI of about 7.5. In some embodiments, the fusion protein has a pI of about 7.9. In some embodiments, the fusion protein has a pI of about 8.0. In some embodiments, the fusion protein has a pI of about 8.4. In some embodiments, the fusion protein has a pI of about 8.5. In some embodiments, the fusion protein has a pI of about 8.8. In some embodiments, the fusion protein has a pI of about 8.9. In some embodiments, any of the pIs referred to above is the calculated or theoretical pI. In some embodiments, any of the pIs referred to above is the experimentally measured pI.

As used herein, the term “about” when referring to dosages encompasses variations of ±10% of a given value or range, as is appropriate to perform the methods disclosed herein.

“Circulating half-life,” as used herein, refers to the time it takes for the blood plasma concentration of a drug to halve its steady-state when circulating in the full blood of an organism. Circulating half-life of a particular agent may vary depending on a multitude of factors including, but not limited to, dosage, formulation, and/or administration route of the agent. One of ordinary skill in the art is able to determine the circulating half-life of an agent using well known methods in the art, such as the method described Chen supra.

In some embodiments, the fusion proteins described herein have high bioavailability. In some embodiments, the fusion proteins have high bioavailability when administered, e.g., intravenously or subcutaneously. In some embodiments, the fusion proteins have high bioavailability when administered subcutaneously. In some embodiments, the fusion proteins have high subcutaneous bioavailability. In some embodiments, the fusion proteins have bioavailability of at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. In some embodiments, the fusion proteins have bioavailability of about 40%, about 45%, about 50%, about 55%, about 60%, about 65%, about 70%, about 75%, about 80%, about 85%, about 90%, about 95%, about 98%, about 99%, or about 100%. In some embodiments, the fusion proteins have bioavailability of 40%, 45%, 50%, 55%, 60%, 65%, 70%, 75%, 80%, 85%, 90%, 95%, 98%, 99%, or 100%. In some embodiments, the fusion proteins have bioavailability of about 40% to about 80%, about 40% to about 75%, about 40% to about 70%, about 40% to about 60%, about 50% to about 80%, about 50% to about 70%, about 50% to about 60%, about 60% to about 80%, or about 70% to about 80. In some embodiments, the fusion proteins have bioavailability of about 50% to about 60% (e.g., 50% to 60%). In some embodiments, the fusion proteins have bioavailability, when administered subcutaneously, of at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. In some embodiments, the fusion proteins have bioavailability, when administered subcutaneously, of about 40%, about 45%, about 50%, about 55%, about 60%, about 65%, about 70%, about 75%, about 80%, about 85%, about 90%, about 95%, about 98%, about 99%, or about 100%. In some embodiments, the fusion proteins have bioavailability, when administered subcutaneously, of 40%, 45%, 50%, 55%, 60%, 65%, 70%, 75%, 80%, 85%, 90%, 95%, 98%, 99%, or 100%. In some embodiments, the fusion proteins have bioavailability, when administered subcutaneously, of about 40% to about 80%, about 40% to about 75%, about 40% to about 70%, about 40% to about 60%, about 50% to about 80%, about 50% to about 70%, about 50% to about 60%, about 60% to about 80%, or about 70% to about 80. In some embodiments, the fusion proteins have bioavailability, when administered subcutaneously, of about 50% to about 60% (e.g., 50% to 60%).

“Bioavailability,” as used herein, refers to the fraction of administered drug that arrives in systemic circulation. Bioavailability of a particular agent may vary depending on a multitude of factors including, but not limited to, dosage, formulation, administration route, and/or properties of the agent. One of ordinary skill in the art is able to determine the bioavailability of an agent using well known methods in the art.

In some embodiments, the fusion proteins have high and/or stable serum levels when administered to a subject, e.g., intravenously, subcutaneously, and/or according to any of the methods described herein. In some embodiments the fusion proteins are present in subject serum at a level of at least about 0.5 μg/mL, at least about 1 μg/mL, at least about 2 μg/mL, at least about 3 μg/mL, at least about 4 μg/mL, at least about 5 μg/mL, at least about 6 μg/mL, at least about 7 μg/mL, at least about 8 μg/mL, or at least about 9 μg/mL 0.5 days, 1 day, 2 days, 3 days, 4 days, 5 days, 6 days, 7 days, 8 days, 9 days, 10 days, or more after administration. In some embodiments the fusion proteins are present in subject serum at a level of at least about 0.5 μg/mL (e.g., at least about 1 μg/mL, at least about 2 μg/mL, at least about 3 μg/mL, at least about 4 μg/mL, at least about 5 μg/mL, at least about 6 μg/mL, at least about 7 μg/mL, at least about 8 μg/mL, or at least about 9 μg/mL) for at least 0.5 days, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, or more after administration. In some embodiments the fusion proteins are present in subject serum at a level of at least about 0.5 μg/mL (e.g., at least about 1 μg/mL, at least about 2 μg/mL, at least about 3 μg/mL, at least about 4 μg/mL, at least about 5 μg/mL, at least about 6 μg/mL, at least about 7 μg/mL, at least about 8 μg/mL, or at least about 9 μg/mL) for at least 0.5 days, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, or more after intravenous administration. In some embodiments the fusion proteins are present in subject serum at a level of at least about 0.5 μg/mL (e.g., at least about 1 μg/mL, at least about 2 μg/mL, at least about 3 μg/mL, at least about 4 μg/mL, at least about 5 μg/mL, at least about 6 μg/mL, at least about 7 μg/mL, at least about 8 μg/mL, or at least about 9 μg/mL) for at least 0.5 days, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, or more after subcutaneous administration.

### Vectors and Host Cells

The disclosure also provides nucleic acid molecules that encode any of the fusion proteins or peptides described herein. In some embodiments, the nucleic acid molecules described herein are DNA molecules. In some embodiments, the nucleic acid molecules described herein are RNA molecules.

The nucleic acid molecules described herein can be transcribed from a promoter in an expression vector. In some embodiments, the vector is a non-viral vector. Exemplary non-viral vectors include, but are not limited to, plasmid DNA, transposons, episomal plasmids, minicircles, ministrings, and oligonucleotides (e.g., mRNA, naked DNA). In some embodiments, the vector is a DNA plasmid vector.

In some embodiments, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV2, 3, 5, 6, 8, 9), retrovirus vectors (MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, M1), flavivirus (e.g., Kunjin, West Nile, Dengue virus), rhabdovirus vectors (e.g., rabies virus, VSV), measles virus vector (e.g., MV-Edm), Newcastle disease virus vectors, poxvirus vectors (e.g., VV), measles virus, and picornavirus vectors (e.g., Coxsackievirus).

In some embodiments, the vector or expression cassette comprises one or more additional elements. Additional elements include, but are not limited to, promoters, enhancers, polyadenylation (polyA) sequences, and selection genes.

In some embodiments, the vector comprises a polynucleotide sequence that encodes an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or 99% identical to an amino acid sequence recited in any of Tables 1-8. In some embodiments, the vector comprises a polynucleotide sequence that encodes an amino acid sequence that comprises or consists of an amino acid sequence recited in any of Tables 1-8. In some embodiments, the vector comprises a polynucleotide sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or 99% identical to a sequence recited in Table 9, below. In some embodiments, the vector comprises a polynucleotide sequence that comprises or consists of a sequence recited in Table 9, below.

TABLE 9
Nucleotide Sequences Encoding Fusion Proteins and Peptide Components

| SEQ | |
|---|---|
| ID | |
| NO: | Sequence |
| 410 | GACAAGACCCATACATGTCCGCCTTGTCCTGCGCCTGAGGCAGCAGGCG |
| | GACCATCAGTCTTCTTGTTTCCCCCCAAGCCGAAGGACACCCTTATGATC |
| | TCACGCACCCCCGAAGTAACTTGTGTAGTCGTTGATGTCTCACACGAAG |
| | ACCCGGAAGTAAAGTTTAATTGGTATGTCGATGGTGTTGAGGTCCACAA |
| | CGCTAAAACGAAACCGCGGGAAGAACAATACAACTCCACATATCGAGT |
| | AGTCTCCGTCCTGACTGTTCTTCACCAGGACTGGCTGAATGGTAAAGAAT |
| | ACAAATGTAAAGTGAGTAACAAGGCCCTTGCAGCACCCATCGAGAAGAC |
| | GATATCCAAAGCCAAGGGGCAACCGCGCGAGCCACAAGTTTACACGCTC |
| | CCACCCTCAAGAGACGAACTCACCAAAAATCAAGTGTCCCTGACATGTC |
| | TGGTGAAAGGATTCTATCCCAGCGACATAGCTGTAGAATGGGAGAGTAA |
| | TGGCCAACCCGAAAACAATTACAAAACTACCCCCCCGGTTTTGGATAGT |
| | GATGGTTCATTCTTCCTCTATAGTAAACTTACCGTGGATAAGTCTCGGTG |
| | GCAGCAGGGGAACGTGTTTAGCTGTTCAGTCCTCCATGAGGCACTCCAT |
| | AGTCACTATACGCAAAAGTCATTGTCCCTTTCTCCGGGCAAGGGCGGGT |
| | CAGACTCCTGGCAGGAAGAGGTAATTAAGCTTTGTGGGCGAGAACTCGT |
| | TAGGGCACAGATAGCAATCTGCGGGAAAAGTACAGCTTCCGATGCTGCC |
| | GGGGCTGACGCCAATGCGGGAGCACGCCAGCTCTACTCAGCCCTCGCCA |
| | ACAAGTGTTGTCATGTAGGTTGCACCAAAAGAAGTCTGGCACAGTTTTG |
| | C |
| 411 | GACAAGACGCATACTTGTCCTCCCTGCCCAGCTCCCGAAGCGGCTGGGG |
| | GGCCCTCTGTCTTTCTGTTTCCGCCTAAGCCCAAGGACACGCTCATGATA |
| | AGTCGCACTCCGGAAGTCACCTGTGTTGTCGTCGATGTTAGCCATGAAG |
| | ATCCAGAGGTGAAATTTAACTGGTACGTCGACGGAGTGGAGGTTCACAA |
| | TGCTAAAACCAAACCGCGAGAAGAGCAATACAATTCCACGTATAGGGTC |
| | GTCTCCGTCCTGACAGTACTCCATCAGGATTGGCTGAATGGAAAAGAAT |
| | ACAAGTGCAAGGTTTCCAATAAAGCCTTGGCCGCACCTATTGAGAAAAC |
| | GATATCAAAAGCTAAGGGACAACCTCGGGAGCCGCAAGTATATACACTC |
| | CCCCCTTCTAGGGACGAACTGACAAAAAACCAAGTTAGTCTGACTTGTC |
| | TTGTGAAAGGTTTTTACCCGAGTGATATAGCCGTAGAATGGGAGAGCAA |
| | TGGCCAGCCCGAAAACAATTACAAAACAACTCCCCCAGTATTGGACAGT |
| | GACGGGTCATTTTTTTTGTATTCTAAATTGACCGTAGACAAGTCACGCTG |
| | GCAACAAGGGAATGTATTTAGCTGTTCCGTCCTTCATGAGGCGCTCCATA |
| | GCCATTACACTCAGAAGTCTTTGTCACTGTCACCGGGCAAGGGTGGTTCT |
| | GATTCATGGCAAGAGGAAGTGATTAAGCTGTGCGGTCGGGAGTTGGTAA |
| | GAGCTCAAATTGCGATTTGTGGCAAGAGCACTGCGTCCGATGCCGCAGG |
| | TGCTAATGCCGACGCCGGTGCGAGACAGCTTTATTCTGCGCTGGCCAAC |
| | AAGTGCTGCCACGTCGGATGCACCAAACGGAGCCTTGCTCAGTTTTGC |
| 412 | GACAAAACTCATACTTGTCCACCATGCCCAGCCCCCGAGGCGGCTGGCG |
| | GCCCCAGCGTATTCCTTTTCCCCCCAAAACCTAAGGACACGCTTATGATA |
| | TCTAGAACCCCGGAGGTCACATGTGTCGTCGTAGACGTAAGTCACGAAG |
| | ATCCTGAAGTCAAGTTTAACTGGTACGTCGATGGAGTCGAGGTCCATAA |
| | TGCTAAAACGAAGCCTCGCGAAGAACAGTATAATTCTACCTATCGCGTA |
| | GTCTCTGTCCTCACCGTCTTGCATCAAGACTGGTTGAACGGCAAGGAGTA |
| | CAAGTGTAAGGTTTCAAACAAAGCCCTTGCCGCGCCGATAGAGAAAACA |
| | ATTAGCAAAGCGAAGGGGCAGCCGAGAGAGCCGCAAGTGTATACCCTTC |
| | CTCCTAGTAGAGACGAGTTGACCAAAAACCAGGTGTCACTTACATGCCT |
| | CGTGAAAGGCTTCTACCCGAGTGATATTGCAGTCGAGTGGGAATCCAAC |
| | GGCCAGCCCGAGAATAACTACAAAACGACGCCGCCCGTACTGGACAGTG |
| | ATGGAAGTTTTTTTTTGTACTCAAAACTCACGGTTGACAAAAGTCGGTGG |
| | CAGCAAGGGAACGTTTTTAGCTGCTCTGTCCTCCATGAAGCACTCCATTC |
| | TCATTATACCCAGAAGTCTCTGTCTCTCTCCCCTGGTAAGGGAGGTTCTG |
| | ACAGTTGGCAGGAAGAGGTAATAAAACTCTGCGGTCGAGAGCTTGTTCG |
| | AGCACAAATTGCTATATGTGGAAAATCTACCGCTTCAGACGCCGCCGGA |
| | GCTGATGCGGATGCCGGGGCTCGCCAGCTCTATAGCGCCTTGGCCAACA |
| | AATGTTGTCACGTTGGCTGCACGAAGCGCTCCCTGGCTCAGTTTTGC |
| 413 | GATAAAACCCACACTTGCCCACCTTGCCCTGCGCCGGAAGCCGCCGGAG |
| | GACCTAGTGTTTTCCTCTTTCCCCCTAAGCCCAAAGACACGTTGATGATC |
| | TCTCGGACACCGGAAGTAACTTGTGTCGTTGTGGATGTGTCACATGAGG |
| | ATCCCGAGGTGAAATTTAATTGGTACGTTGACGGCGTGGAGGTGCATAA |
| | CGCAAAGACTAAACCACGCGAGGAGCAGTATAATTCTACATACCGGGTT |
| | GTCTCAGTTCTCACAGTTCTTCATCAGGATTGGTTGAATGGAAAGGAGTA |
| | CAAATGCAAAGTGTCCAACAAAGCGCTTGCTGCGCCGATTGAAAAGACG |
| | ATTTCAAAGGCAAAAGGGCAGCCCCGCGAACCCCAAGTATATACTTTGC |
| | CTCCCTCACGCGATGAACTGACTAAGAACCAGGTGAGCCTGACTTGTTT |
| | GGTTAAGGGTTTTTATCCAAGTGACATTGCTGTTGAATGGGAGTCCAATG |
| | GCCAGCCTGAGAATAACTACAAAACGACACCTCCTGTACTTGACAGCGA |
| | CGGCTCCTTTTTTCTTTATTCAAAACTCACAGTGGACAAATCCAGGTGGC |
| | AGCAGGGTAACGTCTTTTCTTGCAGCGTGCTCCACGAAGCTTTGCATTCA |
| | CATTATACGCAAAAATCCTTGTCATTGTCCCCAGGTAAGGGCGGAAGCG |
| | ACTCATGGCAAGAAGAAGTCATTAAACTTTGTGGACGGGAGCTGGTTAG |
| | GGCACAGATTGCTATTTGTGGTAAGTCTACGGCTAGTGACGCAGCGGGC |
| | GCCGAAGCGGAAGCTGGTGCAAGGCAGCTTTACTCTGCTCTGGCTAACA |
| | AGTGTTGTCACGTTGGGTGTACCAAGCGGTCCCTTGCGCAATTCTGC |
| 414 | GATAAAACTCATACTTGCCCCCCCTGCCCCGCGCCTGAAGCTGCAGGGG |
| | GGCCATCAGTCTTCTTGTTTCCACCAAAACCTAAGGATACTCTCATGATT |
| | AGCCGGACCCCTGAGGTGACATGTGTTGTGGTCGATGTATCTCATGAAG |
| | ATCCCGAAGTAAAATTTAACTGGTACGTAGACGGGGTTGAAGTTCATAA |
| | CGCGAAAACGAAACCTCGGGAGGAGCAATATAATAGCACGTATAGAGT |
| | TGTTTCAGTCCTTACAGTTCTCCACCAAGACTGGCTGAATGGCAAGGAGT |
| | ATAAGTGTAAAGTATCCAATAAAGCCTTGGCTGCGCCAATCGAGAAGAC |
| | GATCAGCAAAGCCAAAGGTCAGCCTCGCGAACCGCAGGTCTATACATTG |
| | CCCCCTTCACGCGACGAACTCACGAAAAATCAAGTCTCTTTGACTTGCCT |
| | TGTGAAAGGCTTCTACCCCTCCGATATTGCCGTCGAATGGGAAAGCAAT |
| | GGACAGCCGGAAAATAATTACAAAACGACACCCCCCGTGTTGGATTCCG |
| | ATGGGTCCTTCTTCCTCTATTCCAAGCTGACGGTCGATAAGTCTCGATGG |
| | CAGCAGGGAAATGTCTTCTCTTGCTCCGTCCTTCATGAGGCATTGCACAG |
| | CCATTATACTCAAAAGAGTCTCTCTCTGTCTCCAGGCAAAGGGGGTTCCG |
| | ACTCTTGGCAAGAAGAGGTCATAAAACTGTGCGGCCGGGAGCTCGTCAG |
| | AGCGCAGATCGCTATATGTGGAAAATCCACCGCGAGTGACGCAGCAGGT |
| | GCACAAGCCGACGCAGGAGCTAGGCAACTGTACTCAGCCCTTGCCAATA |
| | AGTGTTGTCACGTAGGTTGTACTAAACGCTCCCTGGCACAATTTTGT |
| 415 | GATAAGACACACACGTGCCCACCCTGCCCTGCCCCAGAGGCAGCCGGAG |
| | GTCCTAGTGTGTTTCTGTTCCCCCCAAAGCCCAAGGACACCCTTATGATA |
| | TCTAGGACACCAGAAGTTACGTGCGTCGTTGTGGACGTTAGCCACGAAG |
| | ACCCAGAAGTGAAGTTTAATTGGTACGTTGATGGAGTCGAAGTGCACAA |
| | TGCAAAAACAAAACCACGAGAAGAGCAGTATAACAGTACTTATAGAGT |
| | AGTCAGCGTCCTTACTGTATTGCATCAGGATTGGCTGAATGGGAAGGAA |
| | TATAAATGTAAGGTTAGCAATAAAGCCCTTGCGGCTCCTATCGAGAAAA |
| | CTATTAGCAAAGCAAAGGGCCAACCCCGAGAGCCCCAAGTTTATACACT |
| | GCCACCCAGTCGAGATGAGCTGACTAAAAATCAAGTATCCCTGACCTGC |
| | TTGGTTAAGGGGTTTTATCCTAGTGACATCGCGGTTGAGTGGGAATCCAA |
| | CGGCCAACCGGAGAATAATTACAAAACCACGCCACCTGTATTGGATTCC |
| | GATGGTAGCTTCTTTCTCTATAGTAAACTTACAGTCGATAAGTCAAGATG |
| | GCAGCAGGGAAACGTATTTTCATGCTCAGTTCTGCATGAGGCCTTGCACT |
| | CCCATTACACTCAAAAATCACTGAGCCTCAGTCCTGGTAAGGGTGGCTCT |
| | GACTCATGGCAGGAGGAAGTAATCAAGCTGTGTGGGAGGGAATTGGTA |
| | AGGGCTCAGATTGCAATTTGTGGAAAGAGCACAGCGTCTGACGCTGCAG |
| | GTGCCGACGCACAGGCGGGCGCGAGGCAGCTCTACAGTGCTCTTGCGAA |
| | CAAGTGTTGTCATGTAGGTTGCACGAAACGAAGTTTGGCGCAATTCTGT |
| 416 | GATAAAACACACACCTGCCCCCCTTGCCCAGCACCTGAAGCAGCGGGTG |
| | GTCCCAGCGTTTTTCTTTTCCCCCCTAAGCCAAAGGACACGCTCATGATA |
| | AGTCGCACCCCGGAGGTCACCTGCGTCGTTGTTGACGTATCACATGAAG |
| | ATCCTGAGGTGAAGTTTAATTGGTACGTAGATGGTGTTGAGGTCCACAA |
| | CGCAAAGACGAAACCGAGGGAAGAACAGTACAACAGTACTTATCGCGT |
| | AGTCTCCGTTCTGACTGTCCTGCATCAAGATTGGTTGAACGGGAAGGAG |
| | TACAAGTGCAAAGTTAGTAACAAGGCTCTCGCGGCCCCAATTGAGAAGA |
| | CGATATCCAAAGCGAAAGGACAGCCGAGAGAGCCCCAAGTCTACACTCT |
| | GCCCCCTTCCAGGGATGAGCTCACCAAAAATCAGGTCAGTCTCACGTGC |
| | CTGGTTAAGGGATTCTACCCAAGTGATATAGCAGTTGAATGGGAGAGTA |
| | ACGGCCAGCCCGAGAACAACTATAAAACTACACCGCCCGTTCTTGATTC |
| | CGATGGGTCTTTCTTCCTTTATAGTAAGCTCACCGTTGATAAGTCCCGAT |
| | GGCAGCAAGGTAATGTCTTCTCATGTTCAGTTCTTCATGAAGCCCTGCAT |
| | TCCCATTATACACAAAAGAGCTTGTCCTTGTCACCGGGCAAAGGCGGTA |
| | GCGATTCTTGGCAAGAAGAAGTTATAAAGTTGTGCGGTAGGGAACTGGT |
| | ACGCGCTCAAATAGCTATATGCGGTAAGTCTACTGCTTCAGATGCGGCT |
| | GGCGCACAGGCACAGGCCGGTGCTAGACAACTCTATAGTGCGCTGGCCA |
| | ACAAGTGCTGCCATGTGGGGTGTACAAAACGGAGTCTTGCCCAGTTTTG |
| | T |
| 417 | GACAAAACTCATACATGCCCCCCATGCCCAGCACCCGAAGCGGCCGGAG |
| | GTCCGTCTGTCTTTCTGTTTCCGCCGAAACCTAAAGATACGTTGATGATT |
| | AGCAGAACCCCTGAGGTAACATGTGTGGTAGTCGATGTCTCCCATGAGG |
| | ACCCCGAGGTAAAGTTCAATTGGTATGTTGACGGCGTCGAAGTCCATAA |
| | CGCAAAAACGAAGCCCCGAGAGGAGCAATATAACTCTACCTATCGCGTT |
| | GTTTCTGTTTTGACTGTGTTGCACCAGGATTGGCTCAACGGCAAGGAATA |
| | CAAATGTAAAGTGTCCAACAAGGCCCTTGCTGCACCTATCGAAAAAACG |
| | ATTAGTAAGGCAAAGGGACAACCGCGCGAACCACAGGTATATACTTTGC |
| | CGCCTAGCAGAGATGAACTCACCAAGAATCAAGTTTCCCTTACCTGTTTG |
| | GTTAAAGGATTTTACCCGTCTGACATAGCTGTTGAATGGGAGAGCAATG |
| | GTCAGCCGGAAAATAATTATAAAACCACCCCGCCAGTATTGGATTCAGA |
| | TGGGTCCTTTTTCTTGTATTCTAAACTTACCGTGGATAAGTCTAGGTGGC |
| | AACAGGGAAACGTCTTTTCATGTAGTGTACTTCATGAAGCCCTCCATAGT |
| | CACTACACGCAGAAATCCTTGTCTCTTAGTCCGGGTGAAGGTGGGTCTG |
| | ATTCCTGGCAGGAAGAGGTGATAAAGCTCTGTGGTCGGGAACTTGTTAG |
| | GGCGCAGATCGCTATTTGCGGCAAATCTACAGCATCAGATGCCGCCGGA |
| | GCTGATGCGAACGCAGGAGCGAGGCAGCTGTACTCCGCACTTGCTAACA |
| | AGTGTTGCCATGTCGGCTGCACCAAGAGGAGTCTTGCTCAATTCTGC |
| 418 | GATAAGACCCATACATGCCCGCCATGTCCCGCACCCGAGGCAGCGGGTG |
| | GACCCTCTGTCTTTCTGTTCCCTCCAAAGCCAAAAGATACCCTGATGATT |
| | AGCCGAACCCCGGAGGTGACTTGTGTCGTAGTAGATGTCAGTCACGAGG |
| | ATCCCGAAGTAAAGTTTAATTGGTATGTGGACGGTGTGGAGGTACATAA |
| | CGCTAAGACGAAACCCCGAGAGGAACAATACAACTCTACGTACAGGGTC |
| | GTCTCAGTGCTCACGGTCCTGCACCAGGACTGGCTTAATGGGAAGGAAT |
| | ATAAATGCAAAGTCTCTAATAAGGCGCTTGCTGCACCTATTGAAAAAAC |
| | GATTTCTAAGGCGAAGGGACAACCCCGGGAGCCACAAGTCTACACCCTT |
| | CCTCCAAGCAGAGATGAGCTTACGAAAAATCAAGTGTCTCTTACGTGCC |
| | TCGTAAAGGGCTTTTACCCATCCGACATTGCGGTGGAGTGGGAATCAAA |
| | CGGGCAGCCGGAAAATAACTACAAAACAACGCCGCCTGTATTGGATTCC |
| | GACGGCTCTTTCTTCCTTTACAGCAAACTGACAGTCGATAAATCCAGATG |
| | GCAACAAGGGAACGTTTTTTCATGTTCCGTTCTGCATGAAGCCCTTCACA |
| | GTCATTACACCCAAAAGTCACTTTCACTTTCACCGGGCGAGGGGGGGTC |
| | AGACTCCTGGCAAGAGGAAGTTATAAAGTTGTGCGGCAGGGAACTGGTT |
| | AGAGCGCAGATAGCGATTTGCGGAAAATCTACTGCGAGTGATGCTGCGG |
| | GAGCGAATGCGGACGCCGGGGCCCGACAGCTCTATTCCGCACTCGCCAA |
| | TAAGTGCTGCCATGTTGGTTGTACGAAGAGAAGTCTTGCACAATTTTGC |
| 419 | GATAAGACACATACATGCCCCCCCTGCCCGGCTCCCGAAGCTGCCGGGG |
| | GACCGTCAGTGTTTTTGTTTCCGCCCAAGCCGAAGGATACTTTGATGATT |
| | AGTCGGACACCAGAAGTGACATGTGTTGTCGTTGACGTGAGTCACGAGG |
| | ATCCCGAGGTCAAGTTCAACTGGTACGTTGATGGGGTTGAAGTTCACAA |
| | CGCTAAAACGAAACCCCGCGAAGAGCAGTATAACTCCACTTACCGGGTC |
| | GTCAGTGTCCTGACGGTCTTGCACCAGGACTGGCTGAATGGAAAGGAAT |
| | ACAAGTGTAAAGTTTCCAATAAAGCACTGGCCGCCCCGATCGAAAAAAC |
| | AATTTCCAAAGCTAAGGGACAGCCCAGGGAACCGCAAGTTTATACTCTT |
| | CCACCCTCCCGGGATGAACTGACCAAAAACCAAGTGTCTTTGACGTGCC |
| | TCGTAAAGGGCTTCTACCCGTCAGACATAGCTGTCGAATGGGAGTCTAA |
| | TGGACAGCCGGAAAACAATTATAAGACTACACCGCCGGTGCTTGATAGT |
| | GATGGAAGTTTCTTTTTGTACTCCAAACTTACGGTCGATAAAAGCCGGTG |
| | GCAGCAGGGAAACGTATTCAGTTGTAGCGTTCTGCATGAAGCTCTTCATT |
| | CTCACTACACCCAGAAGTCTCTGTCTCTGAGCCCCGGAGAGGGTGGATC |
| | TGATTCTTGGCAGGAAGAAGTGATAAAGTTGTGCGGCCGGGAATTGGTA |
| | CGCGCCCAGATAGCCATTTGCGGGAAGTCTACGGCGAGTGACGCAGCAG |
| | GTGCTGACGCGGACGCTGGTGCTAGACAGCTGTATTCTGCCCTGGCTAAT |
| | AAGTGTTGCCACGTTGGCTGCACCAAGAGATCCCTGGCCCAATTCTGT |
| 420 | GACAAGACACATACTTGTCCCCCCTGCCCAGCTCCAGAAGCTGCCGGAG |
| | GGCCGTCAGTCTTCCTTTTCCCTCCAAAACCTAAGGATACGCTTATGATT |
| | TCTCGAACGCCAGAGGTTACGTGTGTAGTCGTGGACGTTTCCCACGAGG |
| | ATCCTGAGGTCAAGTTTAACTGGTATGTAGACGGGGTTGAGGTCCATAA |
| | TGCCAAGACAAAGCCGCGCGAGGAACAATACAACAGTACATATAGGGT |
| | GGTGAGCGTCCTCACAGTCTTGCATCAAGATTGGCTCAACGGCAAAGAG |
| | TACAAATGTAAGGTTAGCAACAAAGCCCTCGCTGCTCCCATCGAAAAGA |
| | CGATTTCTAAGGCGAAGGGCCAACCACGAGAACCGCAAGTATATACTCT |
| | TCCCCCTTCACGGGACGAGCTGACCAAAAACCAGGTATCCTTGACTTGC |
| | CTGGTCAAAGGATTTTACCCCTCTGATATTGCGGTCGAGTGGGAGAGTA |
| | ATGGGCAACCAGAAAATAATTATAAAACGACCCCCCCGGTACTCGACAG |
| | TGATGGGTCTTTTTTCCTGTATTCTAAGCTTACGGTTGATAAGTCTAGAT |
| | GGCAGCAAGGGAATGTCTTCTCATGTAGTGTTCTGCATGAAGCACTTCAT |
| | TCTCACTATACTCAGAAATCTCTTTCCCTTAGTCCGGGAGAAGGTGGGAG |
| | CGATAGTTGGCAAGAGGAGGTGATAAAACTGTGTGGTCGGGAGCTGGTG |
| | AGAGCCCAAATAGCTATCTGCGGCAAATCAACAGCAAGTGATGCGGCAG |
| | GAGCGGAAGCGGAGGCGGGAGCGCGGCAATTGTATAGTGCCCTTGCTAA |
| | TAAATGCTGTCACGTTGGGTGTACTAAACGATCTCTTGCTCAATTCTGC |
| 421 | GATAAAACCCATACATGTCCTCCGTGTCCCGCTCCAGAAGCCGCTGGCG |
| | GGCCATCTGTGTTTTTGTTCCCCCCCAAGCCTAAGGATACGTTGATGATC |
| | AGCAGGACCCCGGAGGTTACATGCGTAGTAGTTGACGTTTCTCATGAAG |
| | ACCCAGAAGTAAAATTTAACTGGTATGTCGATGGCGTCGAAGTACATAA |
| | TGCTAAAACTAAGCCCAGGGAAGAGCAATACAATTCAACGTACCGAGTT |
| | GTGAGTGTCCTTACGGTCCTGCACCAAGACTGGTTGAACGGCAAAGAGT |
| | ACAAATGCAAAGTGTCTAACAAGGCATTGGCCGCGCCTATAGAAAAGAC |
| | CATTAGCAAAGCAAAAGGGCAGCCTCGGGAACCCCAGGTCTACACGCTG |
| | CCACCTTCCCGAGATGAATTGACGAAAAACCAGGTCTCTTTGACCTGCTT |
| | GGTTAAAGGCTTCTACCCAAGCGACATTGCAGTGGAGTGGGAGTCTAAC |
| | GGGCAACCCGAAAACAACTATAAGACGACTCCCCCTGTTCTTGATTCTG |
| | ATGGGAGTTTTTTTCTGTACAGTAAGTTGACAGTGGATAAATCAAGATG |
| | GCAGCAAGGTAATGTCTTCTCTTGTTCAGTGCTTCACGAAGCATTGCATT |
| | CTCACTACACACAAAAGTCTTTGTCCTTGTCTCCAGGTGAAGGCGGTAGC |
| | GATTCATGGCAAGAAGAAGTCATTAAGCTGTGTGGAAGGGAACTGGTTA |
| | GGGCCCAAATTGCGATATGTGGAAAGTCTACGGCGAGTGATGCGGCCGG |
| | TGCTCAAGCGGATGCGGGTGCTAGACAGTTGTACTCAGCCCTTGCGAAC |
| | AAATGTTGTCACGTTGGCTGTACGAAACGCAGCCTTGCTCAATTCTGC |
| 422 | GATAAAACTCACACATGCCCCCCATGCCCAGCACCGGAAGCTGCCGGAG |
| | GACCGTCTGTATTCCTCTTTCCGCCCAAACCGAAAGACACGTTGATGATT |
| | TCTCGGACTCCCGAGGTAACTTGTGTCGTGGTCGACGTCTCACACGAGG |
| | ACCCGGAGGTCAAATTTAACTGGTATGTCGATGGGGTGGAGGTCCATAA |
| | TGCTAAGACGAAGCCCAGAGAAGAACAGTATAACTCTACTTATAGAGTT |
| | GTAAGCGTGCTCACTGTATTGCACCAGGACTGGCTCAACGGGAAAGAAT |
| | ATAAGTGTAAGGTCTCAAACAAAGCTCTCGCAGCCCCGATAGAGAAAAC |
| | AATATCTAAGGCCAAGGGCCAACCGCGCGAGCCGCAGGTTTATACACTT |
| | CCACCCTCCCGCGATGAGCTGACCAAGAACCAGGTCTCTCTCACCTGTCT |
| | CGTAAAGGGCTTTTATCCCTCCGACATTGCAGTGGAGTGGGAATCAAAC |
| | GGCCAGCCGGAAAATAATTACAAGACCACTCCTCCCGTCCTCGACTCCG |
| | ATGGGTCATTTTTCCTGTACAGTAAGCTCACCGTTGATAAGTCAAGGTGG |
| | CAGCAGGGCAACGTGTTTAGCTGTAGTGTTCTGCATGAGGCGCTCCACA |
| | GTCACTACACCCAGAAAAGTCTGAGCCTTTCCCCAGGTGAGGGTGGTAG |
| | CGATAGCTGGCAGGAGGAAGTAATTAAACTCTGCGGTAGAGAATTGGTA |
| | AGGGCCCAAATTGCCATCTGCGGAAAGAGCACCGCATCAGATGCTGCGG |
| | GCGCGGATGCGCAGGCTGGTGCTAGGCAACTCTACTCTGCCCTGGCGAA |
| | TAAATGTTGCCACGTCGGTTGCACGAAACGAAGTTTGGCTCAATTTTGC |
| 423 | GACAAAACACATACATGCCCCCCTTGCCCGGCTCCCGAGGCCGCCGGTG |
| | GTCCTAGCGTCTTTCTTTTCCCTCCCAAACCCAAAGACACACTTATGATT |
| | AGCAGAACTCCCGAGGTAACATGTGTGGTCGTAGACGTAAGTCACGAAG |
| | ATCCCGAAGTTAAATTCAACTGGTACGTTGATGGTGTGGAAGTTCATAAT |
| | GCAAAAACCAAACCGCGAGAGGAACAGTATAACTCTACCTACCGCGTGG |
| | TCTCAGTGCTGACTGTCCTGCATCAGGACTGGCTCAACGGGAAGGAATA |
| | TAAGTGCAAAGTGAGTAATAAGGCCCTTGCAGCTCCCATAGAAAAGACG |
| | ATATCAAAGGCTAAAGGACAGCCGAGGGAGCCACAGGTGTACACTTTGC |
| | CTCCGAGTAGAGATGAACTCACTAAAAACCAAGTAAGTTTGACATGCCT |
| | GGTCAAAGGTTTTTACCCCAGTGATATAGCGGTTGAGTGGGAGTCCAAT |
| | GGGCAACCGGAGAACAACTATAAGACTACTCCACCTGTCCTGGATAGCG |
| | ATGGAAGTTTTTTTCTTTACTCAAAGCTGACGGTGGATAAGAGTCGATGG |
| | CAGCAGGGCAATGTGTTTAGCTGTTCTGTGCTTCACGAAGCACTTCACTC |
| | TCATTATACCCAGAAGTCATTGAGCCTTTCCCCTGGTGAAGGAGGGTCA |
| | GATTCCTGGCAGGAGGAGGTTATAAAGCTGTGTGGCCGGGAACTCGTGC |
| | GAGCTCAAATTGCGATCTGTGGAAAATCCACCGCTAGTGATGCGGCGGG |
| | AGCACAAGCTCAAGCGGGCGCTCGACAACTTTATAGCGCTTTGGCTAAT |
| | AAGTGCTGCCATGTGGGTTGTACAAAGCGCAGCCTCGCTCAATTTTGC |
| 424 | GATAAGACGCACACTTGCCCTCCTTGCCCGGCACCCGAAGCCGCTGGTG |
| | GGCCTAGTGTATTCCTGTTCCCCCCGAAGCCGAAGGATACTCTTATGATT |
| | TCACGCACGCCCGAGGTTACATGCGTAGTAGTGGACGTATCTCACGAAG |
| | ATCCCGAAGTCAAGTTCAATTGGTATGTCGACGGAGTAGAAGTTCACAA |
| | CGCAAAGACAAAACCGCGGGAAGAGCAATACAACTCCACGTACCGCGT |
| | CGTTTCTGTTCTTACGGTCTTGCACCAGGACTGGCTCAATGGCAAGGAGT |
| | ATAAGTGCAAGGTATCCAACAAGGCCCTTGCCGCACCTATTGAAAAGAC |
| | TATCAGCAAGGCCAAGGGACAGCCAAGGGAGCCTCAAGTCTACACGCTC |
| | CCGCCTAGTAGAGACGAGTTGACAAAGAATCAAGTGAGTTTGACTTGTC |
| | TGGTTAAAGGTTTTTACCCGTCAGATATTGCAGTAGAATGGGAATCTAAC |
| | GGACAACCCGAAAACAACTATAAAACGACGCCTCCTGTGTTGGATTCAG |
| | ATGGGTCATTTTTTCTCTACTCAAAGCTCACGGTAGATAAATCAAGATGG |
| | CAACAAGGCAATGTATTTTCCTGCTCCGTGCTCCACGAGGCTCTGCACAG |
| | CCATTATACGCAAAAGAGTCTGTCTTTGAGCCCAGGTGAGGGTGGCTCC |
| | GATTCCTGGCAGGAGGAAGTAATTAAGTTGTGCGGCAGGGAACTTGTTC |
| | GCGCACAAATAGCCATTTGTGGTCAGAGCACAGCATCAGATGCCGCCGG |
| | AGCCGACGCCAACGCAGGTGCCCGCCAACTTTATTCTGCCCTCGCAAAC |
| | AAATGCTGCCACGTCGGCTGCACGAAGAGGAGCCTCGCCCAATTTTGC |
| 425 | GATAAGACCCATACGTGCCCGCCATGTCCAGCCCCCGAGGCAGCCGGAG |
| | GTCCTTCCGTTTTCCTTTTCCCCCCTAAGCCCAAGGACACTCTGATGATCT |
| | CCCGGACGCCTGAAGTCACTTGCGTAGTCGTAGACGTTTCACATGAGGA |
| | TCCAGAAGTTAAATTTAACTGGTACGTCGATGGCGTCGAGGTCCATAAC |
| | GCGAAAACCAAGCCCAGGGAGGAACAATATAACTCCACCTATAGGGTC |
| | GTGAGTGTGCTCACCGTTTTGCACCAAGACTGGCTCAACGGGAAAGAGT |
| | ACAAATGTAAAGTTTCAAATAAGGCTTTGGCCGCCCCAATAGAGAAGAC |
| | TATATCCAAGGCTAAGGGACAGCCTCGAGAACCGCAGGTATATACGCTT |
| | CCTCCGTCTAGGGATGAACTCACAAAAAACCAGGTTTCTTTGACCTGCTT |
| | GGTAAAGGGATTTTATCCCTCCGACATTGCGGTCGAATGGGAGAGCAAC |
| | GGACAGCCGGAAAACAATTACAAAACGACACCCCCGGTTTTGGACTCTG |
| | ATGGAAGCTTCTTCCTCTATAGTAAGTTGACCGTAGACAAGTCTCGCTGG |
| | CAGCAGGGAAACGTCTTCAGTTGCTCAGTTCTCCATGAGGCGTTGCATA |
| | GTCACTATACACAGAAGAGTCTTAGTTTGTCTCCAGGAGAAGGAGGTTC |
| | TGATTCTTGGCAAGAGGAGGTAATCAAATTGTGTGGCCGAGAACTTGTT |
| | AGAGCTCAGATAGCCATCTGCGGACAGTCTACGGCGTCCGATGCGGCCG |
| | GAGCTAATGCTGACGCAGGTGCGCGACAGCTGTACTCCGCACTGGCGAA |
| | TAAGTGCTGCCACGTGGGATGCACTAAGCGGTCTCTCGCGCAATTCTGT |
| 426 | GATAAAACCCACACCTGTCCACCATGCCCGGCGCCGGAAGCCGCCGGGG |
| | GACCCAGCGTATTTCTTTTCCCCCCCAAGCCCAAAGACACGCTGATGATT |
| | TCACGAACGCCGGAGGTGACTTGCGTGGTAGTGGACGTCTCCCATGAGG |
| | ATCCCGAAGTTAAATTTAATTGGTATGTAGATGGTGTTGAGGTCCATAAT |
| | GCTAAAACAAAGCCGCGGGAAGAGCAATATAACTCCACCTATAGAGTG |
| | GTCTCTGTACTCACTGTCCTGCACCAGGATTGGCTGAATGGGAAAGAGT |
| | ACAAGTGTAAAGTTAGCAACAAAGCGCTCGCCGCGCCTATCGAAAAAAC |
| | GATTTCCAAAGCAAAGGGCCAACCACGAGAACCCCAGGTTTACACCCTG |
| | CCACCCAGTCGAGATGAACTCACTAAGAATCAGGTGTCCCTTACATGCC |
| | TCGTCAAGGGATTCTATCCGAGCGATATAGCGGTGGAATGGGAGAGTAA |
| | CGGTCAACCCGAAAATAACTATAAAACCACTCCGCCGGTACTCGATTCT |
| | GACGGTTCCTTCTTTCTTTATTCCAAACTGACTGTAGACAAATCACGGTG |
| | GCAGCAGGGCAACGTGTTTAGCTGCTCTGTACTCCATGAGGCCTTGCATT |
| | CTCATTATACTCAAAAGAGTCTGAGTCTGAGTCCAGGTGAAGGGGGTTC |
| | CGATTCATGGCAAGAGGAAGTCATTAAACTCTGCGGAAGGGAACTTGTA |
| | AGAGCACAAATCGCGATTTGTGGGCAATCTACCGCATCCGACGCGGCTG |
| | GAGCAGATGCAGATGCCGGAGCGAGGCAGCTGTATTCAGCATTGGCTAA |
| | CAAATGTTGCCATGTTGGATGTACGAAGAGATCACTTGCACAGTTCTGT |
| 427 | GATAAGACTCATACCTGCCCGCCCTGTCCCGCACCCGAGGCTGCCGGAG |
| | GGCCATCAGTGTTCCTTTTCCCACCAAAGCCGAAGGATACACTTATGATC |
| | AGCAGGACACCCGAAGTGACCTGTGTAGTCGTAGACGTGTCCCACGAAG |
| | ACCCCGAAGTAAAATTTAATTGGTATGTCGATGGCGTAGAGGTCCACAA |
| | CGCGAAAACGAAACCCCGCGAAGAACAATATAATTCCACATACCGAGTT |
| | GTCAGCGTCCTCACTGTTCTCCATCAGGACTGGCTGAATGGGAAGGAAT |
| | ATAAGTGCAAGGTCTCAAACAAGGCGCTGGCGGCCCCCATAGAGAAAA |
| | CGATTTCTAAGGCCAAAGGACAGCCACGGGAACCGCAGGTCTATACGCT |
| | CCCACCTAGTAGGGATGAGTTGACCAAGAATCAGGTATCCCTCACATGT |
| | CTCGTCAAGGGATTCTATCCCAGCGACATAGCCGTGGAGTGGGAATCTA |
| | ACGGTCAACCTGAGAATAACTATAAAACAACCCCCCCGGTCCTCGACTC |
| | CGATGGTAGCTTCTTTCTGTATTCCAAACTGACGGTAGATAAAAGCCGAT |
| | GGCAACAGGGTAACGTCTTTAGTTGTTCTGTATTGCACGAGGCGCTCCAT |
| | AGTCACTACACACAGAAGTCTTTGAGCCTCTCACCTGGGGAGGGGGGTA |
| | GCGATTCTTGGCAGGAGGAAGTGATCAAACTGTGCGGCAGGGAACTGGT |
| | CAGAGCACAGATAGCAATATGCGGTCAGAGTACGGCCTCTGACGCCGCC |
| | GGTGCGGAGGCTGAGGCAGGGGCGAGACAGCTCTACAGCGCTCTTGCAA |
| | ATAAGTGTTGTCACGTGGGGTGCACAAAGAGATCCTTGGCGCAATTTTG |
| | T |
| 428 | GATAAGACCCACACATGTCCGCCATGTCCAGCCCCAGAGGCAGCAGGGG |
| | GCCCGTCCGTATTCTTGTTTCCCCCGAAACCCAAAGATACCCTTATGATT |
| | AGTCGAACTCCAGAAGTCACGTGTGTGGTGGTGGACGTATCCCACGAGG |
| | ACCCCGAAGTGAAATTCAATTGGTATGTGGACGGGGTGGAAGTCCATAA |
| | CGCTAAGACGAAGCCCAGAGAGGAGCAGTACAATTCTACCTATCGGGTT |
| | GTATCTGTGCTTACTGTTCTCCATCAAGATTGGCTGAACGGGAAGGAATA |
| | CAAATGTAAAGTTAGTAACAAAGCATTGGCAGCTCCTATCGAAAAGACG |
| | ATAAGCAAGGCTAAAGGTCAACCCCGAGAGCCTCAGGTCTACACTTTGC |
| | CGCCCTCCAGGGATGAGCTTACCAAGAACCAAGTGAGCTTGACGTGTCT |
| | CGTGAAGGGATTCTACCCATCAGATATAGCGGTAGAATGGGAGTCTAAT |
| | GGGCAGCCCGAGAACAACTATAAGACCACCCCTCCCGTTCTTGACTCCG |
| | ACGGTTCCTTTTTCTTGTACTCCAAACTCACGGTCGACAAGTCTAGGTGG |
| | CAGCAAGGCAATGTTTTCAGTTGTTCCGTGCTGCACGAAGCTCTTCATTC |
| | TCACTATACGCAAAAAAGCCTGAGTCTTTCACCTGGAGAGGGGGGTTCC |
| | GATTCTTGGCAGGAAGAAGTCATTAAGCTGTGCGGCAGAGAACTTGTGC |
| | GCGCACAAATTGCTATTTGTGGACAGTCAACTGCATCTGACGCCGCTGG |
| | AGCCCAAGCGGACGCAGGGGCAAGGCAGCTTTATTCAGCGCTTGCGAAT |
| | AAGTGTTGCCATGTGGGTTGCACGAAACGAAGCCTGGCGCAATTTTGT |
| 429 | GATAAGACACATACATGTCCTCCCTGTCCCGCTCCGGAGGCAGCCGGTG |
| | GGCCTTCAGTTTTCTTGTTTCCGCCGAAGCCTAAGGACACGTTGATGATA |
| | TCCCGAACACCAGAGGTCACATGCGTCGTCGTGGACGTCTCACACGAGG |
| | ACCCTGAAGTGAAATTCAACTGGTATGTAGACGGGGTCGAAGTTCACAA |
| | TGCGAAAACTAAACCTCGCGAGGAGCAATATAACTCAACATACCGCGTA |
| | GTGTCCGTCTTGACTGTCCTTCATCAGGATTGGCTGAATGGTAAAGAATA |
| | TAAATGTAAAGTTTCTAATAAAGCGCTTGCGGCACCCATTGAGAAGACA |
| | ATTTCCAAAGCCAAAGGCCAACCCCGAGAGCCTCAGGTATATACGCTGC |
| | CTCCGTCTCGAGATGAGTTGACAAAAAATCAAGTCAGCTTGACTTGTCTT |
| | GTAAAGGGGTTCTATCCGTCAGACATAGCAGTGGAGTGGGAATCCAACG |
| | GGCAACCAGAAAATAATTACAAAACCACTCCGCCCGTGCTTGACTCAGA |
| | TGGGAGCTTCTTCCTTTATAGCAAACTTACGGTAGATAAATCCAGATGGC |
| | AGCAAGGCAACGTATTCAGCTGTAGTGTGCTGCATGAAGCGCTTCACTC |
| | CCATTATACTCAAAAATCTCTTTCTCTGTCACCGGGCGAGGGCGGAAGTG |
| | ATAGTTGGCAGGAAGAGGTCATCAAGCTCTGTGGGAGAGAGCTTGTACG |
| | CGCTCAGATTGCTATATGCGGCCAGTCAACTGCAAGCGATGCAGCGGGT |
| | GCCGATGCCCAAGCGGGGGCACGGCAACTCTACTCAGCCCTCGCGAATA |
| | AATGTTGTCATGTAGGGTGTACTAAGAGAAGCCTCGCGCAATTTTGT |
| 430 | GATAAAACGCATACTTGCCCGCCGTGCCCAGCACCTGAGGCAGCCGGCG |
| | GCCCTAGTGTCTTCTTGTTCCCGCCCAAGCCCAAGGATACACTCATGATC |
| | TCCCGAACGCCAGAGGTCACATGCGTAGTTGTTGACGTTTCCCATGAGG |
| | ACCCTGAAGTGAAATTTAACTGGTACGTCGACGGCGTTGAGGTTCACAA |
| | CGCTAAGACTAAGCCAAGAGAGGAACAGTACAATTCAACTTATAGAGTG |
| | GTGTCTGTATTGACAGTTCTCCATCAGGATTGGCTGAACGGAAAAGAAT |
| | ATAAGTGCAAGGTCTCAAATAAGGCGCTCGCTGCACCCATAGAAAAAAC |
| | CATATCAAAAGCGAAGGGGCAACCAAGAGAACCCCAGGTGTACACGCT |
| | CCCCCCGTCCAGAGATGAACTCACGAAGAATCAAGTGTCACTCACATGT |
| | CTTGTAAAGGGGTTCTACCCCTCTGATATTGCCGTAGAATGGGAAAGCA |
| | ACGGACAGCCCGAGAATAACTACAAGACGACACCGCCAGTTCTTGATTC |
| | TGACGGAAGCTTTTTCCTCTATTCAAAATTGACCGTTGACAAGTCCCGAT |
| | GGCAACAGGGCAACGTTTTCTCATGCTCCGTCCTTCACGAAGCCTTGCAT |
| | TCCCACTATACGCAGAAGAGTCTCTCTTTGAGCCCCGGAGAGGGAGGCA |
| | GTGATTCATGGCAAGAGGAAGTGATCAAACTTTGCGGCAGAGAATTGGT |
| | TAGAGCCCAGATTGCCATTTGTGGACAAAGTACGGCCTCAGATGCTGCG |
| | GGGGCACAAGCTCAGGCGGGCGCACGCCAGTTGTACAGTGCTCTGGCGA |
| | ATAAGTGCTGCCACGTTGGTTGCACCAAGCGATCCTTGGCGCAATTTTGC |
| 431 | GACAAGACTCATACTTGTCCGCCCTGCCCCGCTCCTGAGGCTGCCGGAG |
| | GCCCTTCAGTATTCTTGTTTCCGCCGAAACCGAAGGATACCTTGATGATT |
| | AGTAGGACACCGGAAGTCACCTGCGTAGTGGTGGACGTAAGCCACGAA |
| | GATCCCGAAGTAAAGTTTAATTGGTATGTTGATGGCGTAGAGGTGCATA |
| | ATGCGAAAACCAAACCTAGGGAGGAACAGTACAATAGTACTTACCGCGT |
| | AGTGTCAGTGCTTACCGTGCTGCATCAGGACTGGCTTAATGGGAAGGAA |
| | TACAAATGTAAAGTATCCAATAAAGCGCTGGCGGCTCCCATCGAGAAAA |
| | CGATCTCAAAAGCCAAAGGACAACCACGGGAACCGCAGGTCTATACTCT |
| | GCCACCTTCAAGAGACGAACTTACCAAGAACCAAGTCTCATTGACGTGC |
| | TTGGTAAAAGGTTTTTATCCGTCTGACATCGCTGTTGAATGGGAGTCTAA |
| | CGGCCAGCCGGAGAACAATTACAAAACAACTCCACCAGTCTTGGATTCA |
| | GATGGGTCTTTTTTTTTGTATTCAAAGCTTACCGTTGACAAAAGCCGCTG |
| | GCAACAAGGAAACGTTTTCAGCTGCAGTGTGCTGCACGAAGCGCTCCAC |
| | AGTCATTATACCCAGAAATCTTTGAGCCTGTCTCCAGGGGAAGGTGGGA |
| | GTGACTCTTGGCAAGAAGAGGTTATCAAACTTTGCGGGCGGGAGCTGGT |
| | AAGGGCCCAAATTGCAATATGCGGCAAAAGTACTGCATCTGATGCCGCT |
| | GGGGCCGATGCTAACGCGGGCGCAAGACAACTTTATAGCGCGTTGGCGA |
| | ACAAATGCTGTCATGTGGGATGCACCAAACAAAGTTTGGCGCAATTTTG |
| | T |
| 432 | GATAAAACTCACACGTGTCCGCCATGCCCCGCACCTGAAGCGGCGGGTG |
| | GTCCGAGCGTGTTTTTGTTTCCGCCTAAGCCCAAGGATACCCTGATGATT |
| | AGTCGGACACCCGAAGTAACATGTGTCGTCGTGGATGTAAGTCACGAGG |
| | ATCCCGAAGTGAAATTCAACTGGTATGTGGATGGAGTTGAAGTCCATAA |
| | TGCGAAAACAAAACCGAGAGAGGAACAGTACAACTCAACATACCGGGT |
| | GGTAAGTGTACTGACGGTACTCCACCAGGACTGGCTGAATGGTAAGGAG |
| | TACAAATGCAAAGTTTCAAATAAGGCGCTCGCTGCCCCCATCGAGAAAA |
| | CCATTAGTAAGGCTAAAGGTCAACCTAGGGAGCCACAAGTATATACATT |
| | GCCGCCTTCTAGAGATGAGCTGACCAAAAACCAGGTCAGCCTGACCTGT |
| | TTGGTGAAAGGCTTCTATCCAAGCGACATTGCTGTCGAGTGGGAGTCAA |
| | ATGGGCAGCCGGAAAATAACTATAAAACGACTCCTCCTGTTCTCGACTC |
| | CGATGGTTCATTCTTCCTCTACTCAAAGCTTACCGTGGATAAATCCAGGT |
| | GGCAACAAGGTAACGTGTTCTCATGTTCCGTTCTGCACGAAGCACTGCAT |
| | TCCCATTATACACAAAAATCCCTGAGCCTCTCACCTGGGGAGGGCGGAA |
| | GCGATAGTTGGCAAGAGGAAGTAATAAAGCTGTGTGGCAGGGAACTCGT |
| | AAGGGCTCAGATTGCGATATGTGGAAAAAGCACTGCTTCTGACGCCGCA |
| | GGGGCCAACGCAGATGCTGGCGCCCGACAACTCTATTCTGCGCTTGCGA |
| | ACAAGTGTTGTCATGTAGGATGTACCAAGCAAAGCCTTGCTCAGTTCTGT |
| 433 | GACAAAACCCACACTTGTCCGCCCTGTCCCGCTCCGGAGGCTGCAGGCG |
| | GCCCAAGTGTGTTTCTTTTCCCCCCAAAGCCGAAAGACACCTTGATGATA |
| | TCCCGCACACCCGAAGTGACTTGCGTTGTCGTCGACGTGTCTCATGAGGA |
| | CCCAGAAGTCAAGTTTAATTGGTACGTTGATGGCGTGGAAGTTCACAAT |
| | GCGAAAACTAAGCCCAGAGAGGAGCAATATAACTCAACCTACCGGGTG |
| | GTAAGTGTTCTGACAGTTCTCCACCAGGACTGGTTGAACGGAAAAGAAT |
| | ACAAATGCAAAGTGAGTAACAAAGCCCTGGCTGCCCCTATCGAAAAGAC |
| | CATATCCAAAGCGAAGGGCCAGCCACGGGAACCGCAAGTATATACACTT |
| | CCACCATCTAGAGATGAGCTTACAAAGAACCAGGTGTCCCTTACCTGCC |
| | TTGTCAAAGGCTTCTATCCCTCTGACATCGCAGTGGAGTGGGAGTCCAAC |
| | GGACAACCAGAGAACAACTATAAGACAACGCCGCCAGTACTGGATTCA |
| | GATGGTTCATTCTTCTTGTATTCTAAACTGACTGTTGATAAATCCCGATG |
| | GCAGCAGGGCAACGTTTTTAGTTGTAGTGTTCTGCACGAAGCCCTTCATT |
| | CCCATTATACACAAAAATCTCTTTCCCTCAGCCCAGGCGAGGGAGGAAG |
| | TGACAGTTGGCAAGAGGAGGTGATAAAGCTCTGTGGGAGGGAGCTGGT |
| | ACGCGCACAGATTGCAATCTGCGGAAAGAGCACAGCAAGCGATGCTGCT |
| | GGGGCCGATGCCGATGCTGGCGCTCGACAATTGTATTCAGCTCTTGCTAA |
| | CAAATGCTGTCACGTAGGATGCACTAAACAGAGCCTTGCTCAATTTTGT |
| 434 | GATAAGACCCACACATGTCCACCATGCCCAGCCCCAGAAGCGGCAGGTG |
| | GTCCTTCTGTGTTTCTCTTTCCTCCCAAACCGAAAGATACTCTGATGATA |
| | AGCCGGACCCCAGAAGTTACGTGCGTTGTAGTAGACGTGTCTCACGAGG |
| | ACCCAGAAGTGAAGTTTAACTGGTATGTCGACGGTGTAGAAGTTCATAA |
| | TGCGAAAACAAAGCCCAGGGAAGAACAATATAATTCAACGTACCGGGT |
| | CGTTTCCGTGCTGACAGTTCTGCACCAAGATTGGCTCAACGGGAAAGAG |
| | TACAAATGCAAAGTATCAAATAAGGCCTTGGCTGCGCCGATTGAAAAGA |
| | CGATTTCCAAAGCAAAGGGCCAGCCAAGGGAACCCCAGGTCTATACCCT |
| | CCCTCCTAGCAGAGATGAACTTACAAAAAACCAAGTCTCCCTCACCTGC |
| | CTGGTCAAAGGATTCTATCCCTCAGATATAGCAGTAGAATGGGAAAGTA |
| | ACGGGCAGCCCGAAAACAATTATAAGACCACTCCTCCAGTACTCGATTC |
| | AGACGGTAGCTTCTTTCTGTATTCCAAGCTGACCGTAGATAAAAGTAGGT |
| | GGCAGCAAGGTAATGTCTTCTCATGTAGTGTACTTCATGAGGCGTTGCAT |
| | TCCCATTACACGCAAAAGTCTTTGAGTCTCAGTCCGGGTGAAGGAGGTA |
| | GCGATTCTTGGCAGGAAGAAGTAATTAAGCTGTGCGGCCGGGAGCTCGT |
| | CAGGGCTCAGATAGCTATATGCGGCAAGAGCACGGCCAGTGATGCTGCT |
| | GGTGCAGAGGCTGAAGCAGGTGCCAGGCAGTTGTACAGCGCACTCGCTA |
| | ATAAGTGTTGCCACGTGGGGTGTACAAAGCAATCTTTGGCACAATTCTGT |
| 435 | GATAAGACCCATACTTGTCCTCCGTGCCCGGCACCAGAGGCTGCGGGTG |
| | GCCCATCCGTTTTCCTGTTTCCGCCAAAGCCTAAGGATACTCTGATGATT |
| | TCACGCACACCCGAAGTGACCTGCGTGGTGGTCGACGTATCTCACGAAG |
| | ACCCAGAGGTAAAATTCAATTGGTACGTGGACGGCGTCGAGGTTCATAA |
| | CGCGAAAACTAAGCCGAGAGAAGAGCAGTACAACTCTACGTATCGCGTG |
| | GTGTCCGTACTGACAGTATTGCATCAGGACTGGTTGAATGGCAAGGAGT |
| | ATAAGTGCAAGGTATCTAATAAGGCATTGGCTGCCCCAATAGAGAAAAC |
| | GATCAGCAAAGCAAAGGGGCAGCCGCGCGAGCCGCAGGTATATACACT |
| | TCCACCATCACGGGATGAGTTGACGAAAAATCAAGTCTCTCTCACATGT |
| | CTGGTAAAAGGTTTCTATCCTTCTGATATCGCCGTGGAATGGGAAAGCA |
| | ACGGCCAACCCGAAAACAACTATAAGACGACGCCGCCGGTACTCGACA |
| | GCGACGGAAGCTTTTTCTTGTATTCCAAGTTGACAGTGGACAAGTCTCGA |
| | TGGCAGCAAGGAAACGTGTTCTCATGTTCTGTTCTTCACGAAGCCCTTCA |
| | TAGCCATTATACTCAGAAATCTCTCTCACTCTCCCCAGGTGAAGGGGGA |
| | AGTGACTCTTGGCAAGAAGAAGTCATTAAGCTTTGCGGTCGAGAATTGG |
| | TTCGGGCTCAAATAGCTATTTGTGGCAAGTCCACGGCAAGTGATGCAGC |
| | GGGGGCTCAGGCAGACGCGGGCGCAAGGCAGCTTTATTCCGCACTTGCA |
| | AATAAGTGCTGTCACGTCGGATGTACTAAACAATCACTTGCACAATTCTG |
| | C |
| 436 | GACAAGACACATACATGTCCCCCATGCCCTGCACCCGAAGCTGCTGGGG |
| | GGCCCAGCGTGTTCCTGTTTCCGCCGAAGCCCAAGGACACATTGATGATT |
| | AGTAGAACCCCAGAGGTAACTTGTGTTGTGGTCGATGTGTCACATGAAG |
| | ACCCCGAGGTAAAGTTTAACTGGTATGTGGATGGGGTAGAGGTACATAA |
| | TGCAAAAACCAAGCCGCGGGAGGAGCAGTATAATTCAACCTATCGAGTC |
| | GTGTCAGTCTTGACCGTGCTCCACCAGGACTGGCTTAACGGTAAGGAGT |
| | ATAAATGCAAAGTCAGTAATAAGGCATTGGCCGCCCCCATTGAGAAGAC |
| | CATCAGTAAAGCTAAGGGGCAACCTAGAGAGCCACAGGTTTACACCCTC |
| | CCTCCCTCCCGGGATGAACTCACCAAAAACCAGGTGTCCCTTACTTGTTT |
| | GGTAAAGGGCTTTTATCCTTCTGATATTGCTGTTGAATGGGAGTCTAACG |
| | GGCAACCTGAAAATAACTACAAAACAACTCCCCCCGTTCTGGACTCTGA |
| | TGGGTCATTCTTCCTTTATTCAAAATTGACAGTTGATAAGAGTAGATGGC |
| | AACAAGGCAACGTATTTTCATGTTCTGTGCTCCACGAGGCTCTCCATTCC |
| | CACTACACACAGAAAAGTCTCTCACTGTCCCCAGGAGAGGGCGGGAGCG |
| | ACTCTTGGCAGGAAGAAGTAATCAAGTTGTGTGGCAGGGAACTCGTACG |
| | CGCTCAGATTGCAATATGCGGGAAATCCACGGCAAGTGACGCTGCCGGG |
| | GCCGACGCGCAAGCAGGGGCACGGCAGCTTTACTCCGCCCTCGCAAATA |
| | AATGTTGTCATGTGGGATGCACTAAACAGTCCCTTGCCCAGTTTTGC |
| 437 | GATAAAACCCATACCTGTCCACCATGCCCCGCGCCAGAGGCAGCGGGTG |
| | GTCCAAGCGTTTTCCTTTTTCCACCGAAACCAAAAGATACACTTATGATA |
| | TCAAGGACCCCCGAGGTAACGTGCGTCGTAGTTGACGTTTCTCACGAAG |
| | ATCCCGAGGTGAAATTCAATTGGTACGTAGATGGTGTAGAGGTACACAA |
| | TGCGAAGACAAAACCGCGGGAAGAGCAGTATAATAGCACATACAGAGT |
| | CGTGAGCGTCCTCACCGTACTTCACCAAGATTGGCTGAATGGAAAGGAG |
| | TACAAATGTAAGGTAAGTAATAAAGCACTTGCGGCCCCCATCGAGAAAA |
| | CTATCAGTAAAGCAAAAGGGCAACCACGAGAACCCCAGGTCTACACTTT |
| | GCCACCATCACGGGATGAACTGACAAAAAATCAGGTGTCACTCACTTGC |
| | CTTGTTAAAGGGTTCTATCCTAGTGACATAGCGGTAGAATGGGAGTCTA |
| | ACGGGCAGCCTGAGAACAATTATAAAACTACGCCCCCTGTTCTTGATTCC |
| | GATGGATCATTTTTTCTCTACTCCAAACTCACCGTAGACAAATCCCGCTG |
| | GCAGCAGGGCAACGTGTTTAGTTGCAGCGTTCTTCACGAAGCACTTCACT |
| | CACATTACACACAAAAGTCCCTGAGCTTGAGTCCTGGGGAGGGTGGATC |
| | TGATTCTTGGCAGGAAGAAGTTATAAAACTTTGTGGCAGAGAGTTGGTC |
| | CGCGCACAAATCGCCATATGTGGTAAAAGCACAGCGTCTGACGCGGCGG |
| | GAGCGCAAGCCCAGGCGGGGGCTCGGCAACTCTACTCAGCCCTGGCTAA |
| | CAAGTGCTGTCACGTGGGATGCACTAAACAAAGTCTGGCGCAGTTCTGC |
| 438 | GACAAAACCCATACGTGTCCCCCGTGTCCGGCTCCAGAGGCTGCGGGAG |
| | GACCGTCTGTGTTCTTGTTCCCGCCGAAGCCTAAAGATACGCTGATGATT |
| | AGTCGGACCCCCGAGGTGACCTGCGTGGTAGTAGACGTATCTCATGAAG |
| | ATCCGGAAGTAAAGTTTAACTGGTACGTAGACGGCGTCGAGGTACATAA |
| | TGCCAAGACGAAACCCAGAGAAGAGCAATATAATAGCACTTATCGAGTT |
| | GTAAGCGTATTGACGGTCCTTCACCAGGACTGGTTGAACGGCAAAGAGT |
| | ACAAATGTAAGGTATCCAATAAAGCATTGGCTGCGCCAATTGAAAAGAC |
| | AATTTCCAAAGCGAAGGGGCAACCTCGAGAGCCGCAAGTCTACACGCTG |
| | CCACCGAGTAGGGATGAATTGACTAAGAATCAGGTGAGTCTCACGTGTC |
| | TCGTGAAGGGGTTTTACCCCAGTGATATTGCGGTAGAATGGGAGTCCAA |
| | CGGTCAGCCAGAAAATAATTATAAAACAACGCCCCCTGTATTGGATTCT |
| | GACGGGAGCTTTTTCCTGTACTCAAAACTCACCGTAGATAAGAGTCGCT |
| | GGCAACAGGGCAACGTATTCTCATGTAGCGTTCTGCACGAGGCGCTGCA |
| | CTCTCACTACACACAGAAGAGTTTGAGTTTGTCCCCTGGCGAAGGAGGT |
| | TCTGATTCCTGGCAGGAGGAGGTGATTAAGCTGTGTGGCCGCGAATTGG |
| | TGAGGGCTCAAATTGCTATTTGCGGACAGAGCACAGCGTCCGATGCCGC |
| | CGGCGCAGATGCTAATGCCGGTGCAAGGCAACTGTACTCCGCTCTCGCC |
| | AATAAGTGTTGTCATGTCGGCTGCACCAAGCAATCCCTGGCCCAGTTTTG |
| | C |
| 439 | GACAAGACTCACACTTGTCCCCCATGTCCAGCACCGGAAGCTGCCGGCG |
| | GTCCCTCAGTTTTCCTTTTCCCCCCCAAACCCAAGGACACCCTTATGATTT |
| | CAAGGACACCAGAGGTAACGTGCGTAGTGGTGGACGTCAGTCATGAAG |
| | ACCCAGAGGTAAAGTTTAACTGGTACGTGGATGGGGTAGAGGTTCATAA |
| | TGCTAAAACAAAACCACGCGAGGAACAGTACAATAGTACGTATAGAGT |
| | GGTCTCCGTTCTTACGGTGCTGCATCAGGACTGGCTGAACGGAAAAGAG |
| | TACAAGTGTAAGGTTAGCAATAAGGCGCTGGCGGCCCCAATCGAAAAGA |
| | CGATTTCTAAGGCCAAAGGCCAGCCAAGGGAGCCACAAGTATATACCCT |
| | TCCCCCTTCCCGAGATGAGCTGACTAAGAATCAAGTCAGTCTCACCTGCC |
| | TTGTCAAAGGGTTCTACCCATCCGATATTGCTGTTGAATGGGAGTCTAAT |
| | GGCCAGCCGGAGAACAATTACAAGACAACTCCGCCTGTATTGGATTCCG |
| | ACGGGTCTTTTTTCCTCTATTCAAAACTCACAGTAGACAAAAGTCGATGG |
| | CAGCAAGGTAACGTGTTTTCTTGCTCTGTGTTGCATGAAGCACTTCATTC |
| | TCATTATACTCAAAAATCATTGAGCCTCAGTCCAGGCGAAGGGGGTAGT |
| | GACTCATGGCAGGAGGAGGTAATCAAGCTTTGCGGACGAGAGTTGGTCA |
| | GGGCCCAGATAGCTATTTGTGGTCAGTCCACGGCGAGTGACGCAGCAGG |
| | GGCGAATGCCGATGCAGGAGCAAGACAACTGTATTCTGCTCTGGCCAAC |
| | AAGTGTTGTCATGTAGGGTGTACTAAACAAAGTCTCGCCCAGTTCTGC |
| 440 | GATAAAACCCACACCTGTCCCCCATGTCCGGCTCCCGAAGCAGCGGGGG |
| | GCCCTTCAGTTTTTCTCTTTCCCCCCAAACCGAAAGACACGCTGATGATT |
| | AGCAGAACTCCAGAGGTTACCTGTGTAGTTGTGGACGTTTCACACGAGG |
| | ATCCCGAGGTTAAATTCAACTGGTATGTGGACGGCGTCGAAGTGCATAA |
| | TGCAAAAACAAAGCCCCGGGAAGAACAATATAATAGTACCTATAGGGTC |
| | GTTTCCGTACTGACCGTACTTCATCAAGATTGGCTCAATGGGAAGGAAT |
| | ACAAATGTAAAGTGAGTAATAAAGCCCTGGCGGCACCGATCGAAAAAA |
| | CCATTTCAAAGGCTAAGGGACAACCGCGCGAACCTCAGGTCTATACCTT |
| | GCCCCCTTCACGCGACGAGCTTACGAAGAATCAGGTAAGCCTTACTTGT |
| | CTTGTCAAGGGTTTTTACCCCAGCGACATAGCTGTCGAGTGGGAATCCA |
| | ATGGCCAACCGGAGAATAATTACAAAACTACCCCTCCTGTTCTTGATAG |
| | CGACGGAAGCTTCTTCTTGTATTCCAAACTCACAGTAGATAAAAGTAGG |
| | TGGCAGCAGGGCAATGTATTTTCTTGCAGCGTCCTGCATGAAGCACTGC |
| | ATAGCCATTATACTCAAAAGTCCCTGTCTTTGTCTCCTGGAGAGGGCGGA |
| | AGCGATTCTTGGCAAGAGGAAGTTATTAAGCTGTGCGGGCGCGAACTTG |
| | TGAGGGCTCAAATAGCGATATGTGGTCAGAGCACCGCTAGCGATGCGGC |
| | TGGTGCAGACGCCGATGCCGGTGCTAGGCAACTTTACAGTGCACTTGCG |
| | AATAAGTGCTGTCACGTCGGATGTACTAAACAAAGCCTCGCCCAGTTCT |
| | GC |
| 441 | GATAAAACACATACTTGCCCTCCTTGTCCGGCTCCCGAAGCCGCAGGTG |
| | GACCTTCCGTCTTTCTTTTCCCACCCAAACCTAAAGACACTTTGATGATT |
| | AGCCGGACCCCCGAGGTAACCTGTGTCGTAGTTGACGTTTCCCATGAAG |
| | ACCCCGAAGTTAAGTTCAACTGGTATGTCGACGGCGTCGAGGTGCACAA |
| | CGCGAAGACTAAGCCAAGAGAGGAGCAATACAATTCAACTTACAGGGT |
| | CGTGTCCGTCTTGACAGTGCTTCATCAAGACTGGCTTAATGGAAAGGAA |
| | TACAAATGTAAAGTCTCCAACAAGGCTCTCGCAGCGCCCATTGAGAAAA |
| | CGATATCCAAAGCGAAGGGTCAACCAAGAGAACCCCAGGTTTACACCCT |
| | CCCCCCTAGTCGGGACGAGCTTACGAAGAACCAGGTCAGTTTGACATGC |
| | CTGGTGAAAGGCTTCTATCCGTCAGACATCGCCGTAGAGTGGGAAAGCA |
| | ACGGGCAACCCGAGAACAACTATAAGACGACTCCCCCGGTGTTGGATAG |
| | CGATGGCTCTTTCTTCCTGTACTCTAAGCTGACCGTAGATAAATCCAGGT |
| | GGCAACAGGGGAACGTGTTTTCATGCTCAGTGCTCCATGAAGCCCTCCA |
| | TTCACACTATACACAAAAGTCTTTGTCACTGTCCCCCGGTGAAGGCGGCA |
| | GTGATAGCTGGCAAGAAGAAGTCATAAAGCTCTGTGGTCGCGAGCTTGT |
| | TAGGGCCCAAATTGCGATCTGTGGTCAGTCAACGGCTTCTGACGCCGCC |
| | GGAGCGGAAGCCGAGGCGGGTGCTCGGCAATTGTATTCAGCACTGGCGA |
| | ACAAATGTTGCCATGTTGGTTGTACTAAACAAAGCCTGGCCCAGTTTTGC |
| 442 | GACAAGACTCACACTTGTCCACCCTGTCCAGCACCTGAAGCCGCTGGTG |
| | GACCATCTGTCTTTCTGTTCCCCCCTAAACCAAAGGATACACTTATGATC |
| | AGCAGAACACCTGAAGTCACATGCGTTGTGGTAGACGTTTCCCACGAGG |
| | ATCCTGAAGTGAAGTTTAACTGGTACGTGGATGGCGTTGAGGTTCATAA |
| | TGCCAAGACGAAACCTCGGGAGGAGCAGTATAATTCTACTTATAGGGTG |
| | GTAAGCGTACTGACAGTCCTCCATCAAGACTGGTTGAACGGGAAGGAAT |
| | ACAAGTGTAAAGTTTCCAACAAAGCTCTGGCGGCGCCTATAGAAAAGAC |
| | AATATCAAAAGCGAAAGGGCAACCCAGAGAGCCTCAAGTATATACATTG |
| | CCCCCTAGCAGAGACGAATTGACGAAAAATCAGGTCTCTCTCACGTGCC |
| | TCGTGAAGGGCTTCTATCCTAGTGATATAGCTGTGGAATGGGAATCCAA |
| | TGGACAGCCAGAAAACAACTACAAGACCACGCCCCCCGTCTTGGATTCC |
| | GACGGGTCATTCTTCCTGTACAGCAAGCTGACTGTCGACAAGAGTCGAT |
| | GGCAACAGGGCAACGTCTTTAGCTGCAGCGTCCTGCACGAAGCTCTGCA |
| | TAGTCATTACACCCAAAAGTCCCTTTCTCTCTCCCCTGGTGAAGGCGGTT |
| | CCGATTCATGGCAAGAAGAAGTAATTAAGCTCTGTGGACGAGAGCTTGT |
| | CCGAGCACAAATTGCGATCTGCGGGCAGAGTACCGCATCTGACGCTGCT |
| | GGCGCGCAGGCAGATGCGGGTGCACGGCAGCTTTATTCAGCTCTCGCCA |
| | ACAAGTGTTGTCATGTGGGGTGTACAAAGCAGAGCCTTGCCCAGTTTTGT |
| 443 | GATAAGACCCATACGTGTCCCCCTTGCCCTGCACCCGAGGCGGCTGGGG |
| | GCCCTTCCGTATTCTTGTTTCCTCCTAAGCCCAAAGATACCTTGATGATA |
| | AGTCGAACGCCAGAAGTGACTTGCGTTGTTGTGGATGTCTCCCACGAGG |
| | ATCCAGAAGTCAAATTTAACTGGTATGTCGATGGGGTCGAAGTGCATAA |
| | TGCTAAAACGAAACCCAGAGAGGAACAATACAATTCAACATACCGCGTA |
| | GTCAGTGTTCTTACTGTGCTCCATCAGGATTGGCTCAATGGGAAAGAATA |
| | CAAGTGTAAAGTCTCAAATAAAGCATTGGCGGCCCCTATAGAGAAGACC |
| | ATAAGCAAGGCTAAAGGTCAGCCTAGGGAGCCTCAAGTATATACCTTGC |
| | CTCCTAGCAGAGATGAGTTGACCAAGAACCAGGTCAGCCTCACATGCCT |
| | GGTGAAAGGGTTTTACCCATCTGATATTGCCGTCGAGTGGGAAAGTAAT |
| | GGGCAGCCAGAGAACAACTACAAGACGACACCACCGGTACTGGATAGT |
| | GACGGAAGTTTTTTTCTTTACAGTAAGCTCACAGTCGACAAAAGCCGGT |
| | GGCAACAAGGAAATGTATTTTCATGTAGCGTACTTCATGAAGCCCTCCA |
| | CTCTCATTACACGCAGAAGTCACTTTCACTTAGTCCGGGTGAGGGTGGA |
| | AGCGATAGCTGGCAAGAGGAGGTTATCAAGCTCTGTGGACGAGAACTCG |
| | TGAGAGCGCAAATTGCAATCTGCGGGCAGAGCACGGCGAGTGATGCGG |
| | CCGGGGCGGACGCGCAAGCAGGAGCACGACAACTTTATAGTGCTTTGGC |
| | TAATAAATGTTGCCACGTTGGATGTACTAAACAGAGCTTGGCACAGTTTT |
| | GC |
| 444 | GATAAAACGCACACTTGTCCGCCATGTCCGGCACCTGAGGCAGCGGGAG |
| | GACCGTCCGTGTTTCTGTTTCCCCCTAAACCAAAGGACACGCTGATGATC |
| | AGCCGAACACCTGAAGTAACATGCGTGGTCGTTGACGTGTCTCACGAGG |
| | ATCCAGAAGTAAAGTTCAATTGGTATGTTGACGGAGTTGAAGTACATAA |
| | TGCTAAGACTAAACCCCGCGAAGAACAATATAATTCTACGTACAGAGTT |
| | GTATCCGTGCTCACGGTACTTCACCAAGATTGGCTTAACGGGAAAGAAT |
| | ATAAGTGTAAGGTCTCAAATAAGGCCCTGGCTGCTCCGATCGAAAAAAC |
| | GATATCAAAGGCAAAGGGTCAACCTCGGGAGCCTCAAGTATATACCCTC |
| | CCCCCATCTAGGGATGAGCTGACAAAGAACCAAGTTTCACTGACCTGTC |
| | TCGTAAAGGGTTTCTATCCTTCTGACATCGCAGTTGAATGGGAGTCCAAC |
| | GGCCAACCAGAGAACAACTATAAGACGACACCCCCCGTGTTGGACAGTG |
| | ACGGAAGTTTTTTCCTGTACTCCAAGCTGACGGTTGATAAAAGTAGATG |
| | GCAACAAGGAAATGTTTTCAGTTGTTCTGTGTTGCACGAGGCCCTCCACT |
| | CACACTATACCCAAAAAAGTTTGTCTCTGAGTCCCGGTGAAGGCGGGAG |
| | CGATTCATGGCAGGAGGAAGTAATCAAACTTTGTGGGCGAGAACTGGTC |
| | AGGGCGCAAATAGCGATATGTGGGCAAAGCACAGCTTCAGATGCAGCC |
| | GGTGCTCAAGCTCAGGCTGGAGCTCGACAGCTTTATAGCGCCTTGGCTA |
| | ATAAATGTTGTCACGTTGGCTGTACGAAGCAGAGCCTGGCACAGTTCTG |
| | C |
| 445 | GATAAAACCCACACTTGCCCACCTTGCCCTGCGCCGGAAGCCGCCGGAG |
| | GACCTAGTGTTTTCCTCTTTCCCCCTAAGCCCAAAGACACGTTGATGATC |
| | TCTCGGACACCGGAAGTAACTTGTGTCGTTGTGGATGTGTCACATGAGG |
| | ATCCCGAGGTGAAATTTAATTGGTACGTTGACGGCGTGGAGGTGCATAA |
| | CGCAAAGACTAAACCACGCGAGGAGCAGTATAATTCTACATACCGGGTT |
| | GTCTCAGTTCTCACAGTTCTTCATCAGGATTGGTTGAATGGAAAGGAGTA |
| | CAAATGCAAAGTGTCCAACAAAGCGCTTGCTGCGCCGATTGAAAAGACG |
| | ATTTCAAAGGCAAAAGGGCAGCCCCGCGAACCCCAAGTATATACTTTGC |
| | CTCCCTCACGCGATGAACTGACTAAGAACCAGGTGAGCCTGACTTGTTT |
| | GGTTAAGGGTTTTTATCCAAGTGACATTGCTGTTGAATGGGAGTCCAATG |
| | GCCAGCCTGAGAATAACTACAAAACGACACCTCCTGTACTTGACAGCGA |
| | CGGCTCCTTTTTTCTTTATTCAAAACTCACAGTGGACAAATCCAGGTGGC |
| | AGCAGGGTAACGTCTTTTCTTGCAGCGTGCTCCACGAAGCTTTGCATTCA |
| | CATTATACGCAAAAATCCTTGTCATTGTCCCCAGGTAAGGGCGGAAGCG |
| | ACTCATACCAAGAAGAAGTCATTAAACTTTGTGGACGGGAGCTGGTTAG |
| | GGCACAGATTGCTATTTGTGGTAAGTCTACGGCTAGTGACGCAGCGGGC |
| | GCCGAAGCGGAAGCTGGTGCAAGGCAGCTTTACTCTGCTCTGGCTAACA |
| | AGTGTTGTCACGTTGGGTGTACCAAGCGGTCCCTTGCGCAATTCTGC |
| 446 | GATAAAACCCACACTTGCCCACCTTGCCCTGCGCCGGAAGCCGCCGGAG |
| | GACCTAGTGTTTTCCTCTTTCCCCCTAAGCCCAAAGACACGTTGATGATC |
| | TCTCGGACACCGGAAGTAACTTGTGTCGTTGTGGATGTGTCACATGAGG |
| | ATCCCGAGGTGAAATTTAATTGGTACGTTGACGGCGTGGAGGTGCATAA |
| | CGCAAAGACTAAACCACGCGAGGAGCAGTATAATTCTACATACCGGGTT |
| | GTCTCAGTTCTCACAGTTCTTCATCAGGATTGGTTGAATGGAAAGGAGTA |
| | CAAATGCAAAGTGTCCAACAAAGCGCTTGCTGCGCCGATTGAAAAGACG |
| | ATTTCAAAGGCAAAAGGGCAGCCCCGCGAACCCCAAGTATATACTTTGC |
| | CTCCCTCACGCGATGAACTGACTAAGAACCAGGTGAGCCTGACTTGTTT |
| | GGTTAAGGGTTTTTATCCAAGTGACATTGCTGTTGAATGGGAGTCCAATG |
| | GCCAGCCTGAGAATAACTACAAAACGACACCTCCTGTACTTGACAGCGA |
| | CGGCTCCTTTTTTCTTTATTCAAAACTCACAGTGGACAAATCCAGGTGGC |
| | AGCAGGGTAACGTCTTTTCTTGCAGCGTGCTCCACGAAGCTTTGCATTCA |
| | CATTATACGCAAAAATCCTTGTCATTGTCCCCAGGTAAGGGCGGAAGCG |
| | ACTCATTTCAAGAAGAAGTCATTAAACTTTGTGGACGGGAGCTGGTTAG |
| | GGCACAGATTGCTATTTGTGGTAAGTCTACGGCTAGTGACGCAGCGGGC |
| | GCCGAAGCGGAAGCTGGTGCAAGGCAGCTTTACTCTGCTCTGGCTAACA |
| | AGTGTTGTCACGTTGGGTGTACCAAGCGGTCCCTTGCGCAATTCTGC |
| 447 | GATAAAACCCACACTTGCCCACCTTGCCCTGCGCCGGAAGCCGCCGGAG |
| | GACCTAGTGTTTTCCTCTTTCCCCCTAAGCCCAAAGACACGTTGATGATC |
| | TCTCGGACACCGGAAGTAACTTGTGTCGTTGTGGATGTGTCACATGAGG |
| | ATCCCGAGGTGAAATTTAATTGGTACGTTGACGGCGTGGAGGTGCATAA |
| | CGCAAAGACTAAACCACGCGAGGAGCAGTATAATTCTACATACCGGGTT |
| | GTCTCAGTTCTCACAGTTCTTCATCAGGATTGGTTGAATGGAAAGGAGTA |
| | CAAATGCAAAGTGTCCAACAAAGCGCTTGCTGCGCCGATTGAAAAGACG |
| | ATTTCAAAGGCAAAAGGGCAGCCCCGCGAACCCCAAGTATATACTTTGC |
| | CTCCCTCACGCGATGAACTGACTAAGAACCAGGTGAGCCTGACTTGTTT |
| | GGTTAAGGGTTTTTATCCAAGTGACATTGCTGTTGAATGGGAGTCCAATG |
| | GCCAGCCTGAGAATAACTACAAAACGACACCTCCTGTACTTGACAGCGA |
| | CGGCTCCTTTTTTCTTTATTCAAAACTCACAGTGGACAAATCCAGGTGGC |
| | AGCAGGGTAACGTCTTTTCTTGCAGCGTGCTCCACGAAGCTTTGCATTCA |
| | CATTATACGCAAAAATCCTTGTCATTGTCCCCAGGTAAGGGCGGAAGCG |
| | ACTCACTTCAAGAAGAAGTCATTAAACTTTGTGGACGGGAGCTGGTTAG |
| | GGCACAGATTGCTATTTGTGGTAAGTCTACGGCTAGTGACGCAGCGGGC |
| | GCCGAAGCGGAAGCTGGTGCAAGGCAGCTTTACTCTGCTCTGGCTAACA |
| | AGTGTTGTCACGTTGGGTGTACCAAGCGGTCCCTTGCGCAATTCTGC |
| 448 | GATAAAACCCACACTTGCCCACCTTGCCCTGCGCCGGAAGCCGCCGGAG |
| | GACCTAGTGTTTTCCTCTTTCCCCCTAAGCCCAAAGACACGTTGATGATC |
| | TCTCGGACACCGGAAGTAACTTGTGTCGTTGTGGATGTGTCACATGAGG |
| | ATCCCGAGGTGAAATTTAATTGGTACGTTGACGGCGTGGAGGTGCATAA |
| | CGCAAAGACTAAACCACGCGAGGAGCAGTATAATTCTACATACCGGGTT |
| | GTCTCAGTTCTCACAGTTCTTCATCAGGATTGGTTGAATGGAAAGGAGTA |
| | CAAATGCAAAGTGTCCAACAAAGCGCTTGCTGCGCCGATTGAAAAGACG |
| | ATTTCAAAGGCAAAAGGGCAGCCCCGCGAACCCCAAGTATATACTTTGC |
| | CTCCCTCACGCGATGAACTGACTAAGAACCAGGTGAGCCTGACTTGTTT |
| | GGTTAAGGGTTTTTATCCAAGTGACATTGCTGTTGAATGGGAGTCCAATG |
| | GCCAGCCTGAGAATAACTACAAAACGACACCTCCTGTACTTGACAGCGA |
| | CGGCTCCTTTTTTCTTTATTCAAAACTCACAGTGGACAAATCCAGGTGGC |
| | AGCAGGGTAACGTCTTTTCTTGCAGCGTGCTCCACGAAGCTTTGCATTCA |
| | CATTATACGCAAAAATCCTTGTCATTGTCCCCAGGTAAGGGCGGAAGCG |
| | ACTCAATTCAAGAAGAAGTCATTAAACTTTGTGGACGGGAGCTGGTTAG |
| | GGCACAGATTGCTATTTGTGGTAAGTCTACGGCTAGTGACGCAGCGGGC |
| | GCCGAAGCGGAAGCTGGTGCAAGGCAGCTTTACTCTGCTCTGGCTAACA |
| | AGTGTTGTCACGTTGGGTGTACCAAGCGGTCCCTTGCGCAATTCTGC |
| 449 | GATAAAACCCACACTTGCCCACCTTGCCCTGCGCCGGAAGCCGCCGGAG |
| | GACCTAGTGTTTTCCTCTTTCCCCCTAAGCCCAAAGACACGTTGATGATC |
| | TCTCGGACACCGGAAGTAACTTGTGTCGTTGTGGATGTGTCACATGAGG |
| | ATCCCGAGGTGAAATTTAATTGGTACGTTGACGGCGTGGAGGTGCATAA |
| | CGCAAAGACTAAACCACGCGAGGAGCAGTATAATTCTACATACCGGGTT |
| | GTCTCAGTTCTCACAGTTCTTCATCAGGATTGGTTGAATGGAAAGGAGTA |
| | CAAATGCAAAGTGTCCAACAAAGCGCTTGCTGCGCCGATTGAAAAGACG |
| | ATTTCAAAGGCAAAAGGGCAGCCCCGCGAACCCCAAGTATATACTTTGC |
| | CTCCCTCACGCGATGAACTGACTAAGAACCAGGTGAGCCTGACTTGTTT |
| | GGTTAAGGGTTTTTATCCAAGTGACATTGCTGTTGAATGGGAGTCCAATG |
| | GCCAGCCTGAGAATAACTACAAAACGACACCTCCTGTACTTGACAGCGA |
| | CGGCTCCTTTTTTCTTTATTCAAAACTCACAGTGGACAAATCCAGGTGGC |
| | AGCAGGGTAACGTCTTTTCTTGCAGCGTGCTCCACGAAGCTTTGCATTCA |
| | CATTATACGCAAAAATCCTTGTCATTGTCCCCAGGTAAGGGCGGAAGCG |
| | ACTCATGGCAAGAAGAAGTCATTAAACTTTGTGGACGGGAGCTGGTTAG |
| | GGCACAGATTGCTATTTGTGGTAAGTCTACGGCTAGTGACGCAGCGGGC |
| | GCCGAAGCGGAAGCTGGTGCAAGGCAGCTTTACTCTGCTCTGGCTAACA |
| | AGTGTTGTTACGTTGGGTGTACCAAGCGGTCCCTTGCGCAATTCTGC |
| 450 | GATAAAACCCACACTTGCCCACCTTGCCCTGCGCCGGAAGCCGCCGGAG |
| | GACCTAGTGTTTTCCTCTTTCCCCCTAAGCCCAAAGACACGTTGATGATC |
| | TCTCGGACACCGGAAGTAACTTGTGTCGTTGTGGATGTGTCACATGAGG |
| | ATCCCGAGGTGAAATTTAATTGGTACGTTGACGGCGTGGAGGTGCATAA |
| | CGCAAAGACTAAACCACGCGAGGAGCAGTATAATTCTACATACCGGGTT |
| | GTCTCAGTTCTCACAGTTCTTCATCAGGATTGGTTGAATGGAAAGGAGTA |
| | CAAATGCAAAGTGTCCAACAAAGCGCTTGCTGCGCCGATTGAAAAGACG |
| | ATTTCAAAGGCAAAAGGGCAGCCCCGCGAACCCCAAGTATATACTTTGC |
| | CTCCCTCACGCGATGAACTGACTAAGAACCAGGTGAGCCTGACTTGTTT |
| | GGTTAAGGGTTTTTATCCAAGTGACATTGCTGTTGAATGGGAGTCCAATG |
| | GCCAGCCTGAGAATAACTACAAAACGACACCTCCTGTACTTGACAGCGA |
| | CGGCTCCTTTTTTCTTTATTCAAAACTCACAGTGGACAAATCCAGGTGGC |
| | AGCAGGGTAACGTCTTTTCTTGCAGCGTGCTCCACGAAGCTTTGCATTCA |
| | CATTATACGCAAAAATCCTTGTCATTGTCCCCAGGTAAGGGCGGAAGCG |
| | ACTCATGGCAAGAAGAAGTCATTAAACTTTGTGGACGGGAGCTGGTTAG |
| | GGCACAGATTGCTATTTGTGGTAAGTCTACGGCTAGTGACGCAGCGGGC |
| | GCCGAAGCGGAAGCTGGTGCAAGGCAGCTTTACTCTGCTCTGGCTAACA |
| | AGTGTTGTCTTGTTGGGTGTACCAAGCGGTCCCTTGCGCAATTCTGC |
| 451 | GATAAAACCCACACTTGCCCACCTTGCCCTGCGCCGGAAGCCGCCGGAG |
| | GACCTAGTGTTTTCCTCTTTCCCCCTAAGCCCAAAGACACGTTGATGATC |
| | TCTCGGACACCGGAAGTAACTTGTGTCGTTGTGGATGTGTCACATGAGG |
| | ATCCCGAGGTGAAATTTAATTGGTACGTTGACGGCGTGGAGGTGCATAA |
| | CGCAAAGACTAAACCACGCGAGGAGCAGTATAATTCTACATACCGGGTT |
| | GTCTCAGTTCTCACAGTTCTTCATCAGGATTGGTTGAATGGAAAGGAGTA |
| | CAAATGCAAAGTGTCCAACAAAGCGCTTGCTGCGCCGATTGAAAAGACG |
| | ATTTCAAAGGCAAAAGGGCAGCCCCGCGAACCCCAAGTATATACTTTGC |
| | CTCCCTCACGCGATGAACTGACTAAGAACCAGGTGAGCCTGACTTGTTT |
| | GGTTAAGGGTTTTTATCCAAGTGACATTGCTGTTGAATGGGAGTCCAATG |
| | GCCAGCCTGAGAATAACTACAAAACGACACCTCCTGTACTTGACAGCGA |
| | CGGCTCCTTTTTTCTTTATTCAAAACTCACAGTGGACAAATCCAGGTGGC |
| | AGCAGGGTAACGTCTTTTCTTGCAGCGTGCTCCACGAAGCTTTGCATTCA |
| | CATTATACGCAAAAATCCTTGTCATTGTCCCCAGGTAAGGGCGGAAGCG |
| | ACTCATGGCAAGAAGAAGTCATTAAACTTTGTGGACGGGAGCTGGTTAG |
| | GGCACAGATTGCTATTTGTGGTAAGTCTACGGCTAGTGACGCAGCGGGC |
| | GCCGAAGCGGAAGCTGGTGCAAGGCAGCTTTACTCTGCTCTGGCTAACA |
| | AGTGTTGTCAAGTTGGGTGTACCAAGCGGTCCCTTGCGCAATTCTGC |
| 452 | GATAAAACCCACACTTGCCCACCTTGCCCTGCGCCGGAAGCCGCCGGAG |
| | GACCTAGTGTTTTCCTCTTTCCCCCTAAGCCCAAAGACACGTTGATGATC |
| | TCTCGGACACCGGAAGTAACTTGTGTCGTTGTGGATGTGTCACATGAGG |
| | ATCCCGAGGTGAAATTTAATTGGTACGTTGACGGCGTGGAGGTGCATAA |
| | CGCAAAGACTAAACCACGCGAGGAGCAGTATAATTCTACATACCGGGTT |
| | GTCTCAGTTCTCACAGTTCTTCATCAGGATTGGTTGAATGGAAAGGAGTA |
| | CAAATGCAAAGTGTCCAACAAAGCGCTTGCTGCGCCGATTGAAAAGACG |
| | ATTTCAAAGGCAAAAGGGCAGCCCCGCGAACCCCAAGTATATACTTTGC |
| | CTCCCTCACGCGATGAACTGACTAAGAACCAGGTGAGCCTGACTTGTTT |
| | GGTTAAGGGTTTTTATCCAAGTGACATTGCTGTTGAATGGGAGTCCAATG |
| | GCCAGCCTGAGAATAACTACAAAACGACACCTCCTGTACTTGACAGCGA |
| | CGGCTCCTTTTTTCTTTATTCAAAACTCACAGTGGACAAATCCAGGTGGC |
| | AGCAGGGTAACGTCTTTTCTTGCAGCGTGCTCCACGAAGCTTTGCATTCA |
| | CATTATACGCAAAAATCCTTGTCATTGTCCCCAGGTAAGGGCGGAAGCG |
| | ACTCATGGCAAGAAGAAGTCATTAAACTTTGTGGACGGGAGCTGGTTAG |
| | GGCACAGATTGCTATTTGTGGTAAGTCTACGGCTAGTGACGCAGCGGGC |
| | GCCGAAGCGGAAGCTGGTGCAAGGCAGCTTTACTCTGCTCTGGCTAACA |
| | AGTGTTGTAAAGTTGGGTGTACCAAGCGGTCCCTTGCGCAATTCTGC |
| 453 | GATAAAACCCACACTTGCCCACCTTGCCCTGCGCCGGAAGCCGCCGGAG |
| | GACCTAGTGTTTTCCTCTTTCCCCCTAAGCCCAAAGACACGTTGATGATC |
| | TCTCGGACACCGGAAGTAACTTGTGTCGTTGTGGATGTGTCACATGAGG |
| | ATCCCGAGGTGAAATTTAATTGGTACGTTGACGGCGTGGAGGTGCATAA |
| | CGCAAAGACTAAACCACGCGAGGAGCAGTATAATTCTACATACCGGGTT |
| | GTCTCAGTTCTCACAGTTCTTCATCAGGATTGGTTGAATGGAAAGGAGTA |
| | CAAATGCAAAGTGTCCAACAAAGCGCTTGCTGCGCCGATTGAAAAGACG |
| | ATTTCAAAGGCAAAAGGGCAGCCCCGCGAACCCCAAGTATATACTTTGC |
| | CTCCCTCACGCGATGAACTGACTAAGAACCAGGTGAGCCTGACTTGTTT |
| | GGTTAAGGGTTTTTATCCAAGTGACATTGCTGTTGAATGGGAGTCCAATG |
| | GCCAGCCTGAGAATAACTACAAAACGACACCTCCTGTACTTGACAGCGA |
| | CGGCTCCTTTTTTCTTTATTCAAAACTCACAGTGGACAAATCCAGGTGGC |
| | AGCAGGGTAACGTCTTTTCTTGCAGCGTGCTCCACGAAGCTTTGCATTCA |
| | CATTATACGCAAAAATCCTTGTCATTGTCCCCAGGTGAAGGCGGAAGCG |
| | ACTCATACCAAGAAGAAGTCATTAAACTTTGTGGACGGGAGCTGGTTAG |
| | GGCACAGATTGCTATTTGTGGTAAGTCTACGGCTAGTGACGCAGCGGGC |
| | GCCGAAGCGGAAGCTGGTGCAAGGCAGCTTTACTCTGCTCTGGCTAACA |
| | AGTGTTGTTACGTTGGGTGTACCAAGCGGTCCCTTGCGCAATTCTGC |
| 454 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTGAAGGCGGAAGCGACTCATACCAAGAAGAAGTCATTAAACTTTGTGGACG |
| | GGAGCTGGTTAGGGCACAGATTGCTATTTGTGGTAAGTCTACGGCTAGTGACGCAG |
| | CGGGCGCCGAAGCGGAAGCTGGTGCAAGGCAGCTTTACTCTGCTCTGGCTAACAAG |
| | TGTTGTAAAGTTGGGTGTACCAAGCGGTCCCTTGCGCAATTCTGC |
| 455 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTGAAGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGAGAAGGGGGCGGAAGACAGCTTTACTCTGCTCTGGCTAACAAG |
| | TGTTGTTACGTTGGGTGTACCAAGCGGTCCCTTGCGCAATTCTGC |
| 456 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTGAAGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGAGAAGGGGGCGGAAGACAGCTTTACTCTGCTCTGGCTAACAAG |
| | TGTTGTAAAGTTGGGTGTACCAAGCGGTCCCTTGCGCAATTCTGC |
| 457 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTGAAGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGATCTGGGGGCGGAAGACAGCTTTACTCTGCTCTGGCTAACAAG |
| | TGTTGTTACGTTGGGTGTACCAAGCGGTCCCTTGCGCAATTCTGC |
| 458 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTGAAGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGATCTGGGGGCGGAAGACAGCTTTACTCTGCTCTGGCTAACAAG |
| | TGTTGTAAAGTTGGGTGTACCAAGCGGTCCCTTGCGCAATTCTGC |
| 459 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGCTAGTGACGCAG |
| | CGGGCGCCGAAGCGGAAGCTGGTGCAAGGCAGCTTTACTCTGCTCTGGCTAACAAG |
| | TGTTGTTACGTTGGGTGTACCAAGCAATCCCTTGCGCAATTCTGC |
| 460 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGCTAGTGACGCAG |
| | CGGGCGCCGAAGCGGAAGCTGGTGCAAGGCAGCTTTACTCTGCTCTGGCTAACAAG |
| | TGTTGTAAAGTTGGGTGTACCAAGCAATCCCTTGCGCAATTCTGC |
| 461 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGAGAAGGGGGCGGAAGACAGCTTTACTCTGCTCTGGCTAACAAG |
| | TGTTGTTACGTTGGGTGTACCAAGCAATCCCTTGCGCAATTCTGC |
| 462 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGAGAAGGGGGCGGAAGACAGCTTTACTCTGCTCTGGCTAACAAG |
| | TGTTGTAAAGTTGGGTGTACCAAGCAATCCCTTGCGCAATTCTGC |
| 463 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGATCTGGGGGCGGAAGACAGCTTTACTCTGCTCTGGCTAACAAG |
| | TGTTGTTACGTTGGGTGTACCAAGCAATCCCTTGCGCAATTCTGC |
| 464 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGATCTGGGGGCGGAAGACAGCTTTACTCTGCTCTGGCTAACAAG |
| | TGTTGTAAAGTTGGGTGTACCAAGCAATCCCTTGCGCAATTCTGC |
| 465 | GATAAAACACACACGTGTCCCCCCTGCCCGGCTCCAGAGGCGGCTGGTGGTCCCAG |
| | CGTATTCTTGTTTCCTCCCAAACCTAAGGATACGCTCATGATATCCCGCACCCCAG |
| | AAGTTACGTGTGTAGTCGTCGACGTCAGTCACGAAGATCCAGAGGTCAAATTTAAC |
| | TGGTATGTCGACGGAGTAGAGGTCCACAATGCGAAAACCAAGCCCAGAGAAGAGCA |
| | GTACAACTCCACGTATCGCGTCGTCTCCGTCCTCACCGTACTCCATCAAGATTGGC |
| | TGAATGGGAAAGAGTATAAATGCAAAGTATCTAACAAGGCTCTGCCAGCTCCGATA |
| | GAAAAGACTATATCAAAGGCCAAGGGGCAGCCAAGGGAGCCTCAAGTCTATACTTT |
| | GCCCCCATCTCGGGATGAGCTTACGAAAAACCAGGTCAGCCTTACCTGTCTTGTTA |
| | AAGGTTTTTATCCGAGTGACATCGCAGTGGAATGGGAATCTAATGGTCAACCTGAA |
| | AACAATTACAAAACCACACCGCCAGTATTGGACAGCGATGGTAGTTTTTTTCTTTA |
| | CTCAAAACTGACTGTAGATAAAAGCAGATGGCAGCAGGGCAATGTCTTTTCATGTA |
| | GCGTTATGCATGAGGCTCTTCACAACCACTATACCCAAAAGTCATTGTCTCTTAGT |
| | CCCGGAAAGGGCGGAAGTGATTCTTGGAAGGAGGAGGTAATCAAGTTGTGCGGGCG |
| | AGAGTTGGTACGGGCACAGATCGCGATATGCGGAAAATCCACAGGTGGGGGCGAAG |
| | GAGGAGGTGAGGGTGGAGGTGAAGGACGACAGTTGTATTCCGCCTTGGCAAACAAG |
| | TGTTGCCATGTGGGTTGCACAAAACGCAGTCTTGCCCGCTTCTGT |
| 466 | GATAAGACACATACATGCCCTCCCTGTCCGGCTCCAGAGGCAGCCGGGGGTCCATC |
| | AGTCTTCCTTTTTCCGCCTAAACCTAAGGATACACTGATGATCTCTCGAACACCGG |
| | AGGTCACTTGTGTTGTCGTTGACGTATCACATGAGGATCCCGAAGTAAAGTTCAAC |
| | TGGTATGTCGATGGTGTGGAGGTTCATAATGCTAAAACTAAACCACGGGAGGAGCA |
| | ATATAATTCCACATATAGGGTCGTGAGCGTGTTGACGGTGCTTCATCAAGACTGGC |
| | TTAATGGGAAGGAATATAAATGCAAAGTGTCAAATAAAGCACTTCCTGCGCCAATC |
| | GAGAAAACAATTAGTAAGGCAAAGGGGCAGCCGCGAGAACCTCAGGTGTACACCTT |
| | GCCGCCTTCTAGAGACGAGCTCACAAAGAACCAAGTTTCCCTGACTTGCCTCGTTA |
| | AGGGGTTTTATCCGTCCGATATAGCCGTGGAGTGGGAGTCAAACGGCCAACCGGAA |
| | AATAATTACAAAACGACACCCCCAGTATTGGATAGTGACGGCTCTTTTTTCCTTTA |
| | TTCTAAGCTGACTGTGGACAAAAGCCGCTGGCAGCAGGGCAATGTCTTTTCATGCA |
| | GCGTAATGCATGAAGCCCTGCACAACCACTACACGCAAAAATCCCTTTCCTTGTCA |
| | CCCGGCAAGGGCGGCTCTGACTCCTGGAAAGAGGAAGTTATAAAACTCTGTGGCCG |
| | AGAACTTGTTCGAGCTCAAATCGCGATTTGTGGTAAGTCAACGGGTGGGGGCGAAG |
| | GTGGAGGCGAGGGTGGGGGAGAAGGAGGAGGCCAGTTGTACTCAGCTCTTGCAAAT |
| | AAGTGTTGCCACGTTGGTTGTACGAAGCGGAGCCTTGCTCGCTTCTGC |
| 467 | GACAAAACACATACTTGTCCGCCTTGCCCGGCACCCGAAGCGGCCGGCGGACCCAG |
| | TGTCTTTCTCTTCCCACCCAAACCGAAAGACACTCTGATGATTTCCAGGACGCCTG |
| | AAGTGACCTGCGTTGTAGTTGATGTATCACACGAGGATCCCGAGGTCAAGTTCAAT |
| | TGGTATGTAGATGGGGTGGAGGTCCATAATGCAAAGACGAAGCCACGGGAGGAACA |
| | GTACAACTCTACGTACAGAGTTGTCAGTGTTTTGACCGTCCTTCATCAGGATTGGC |
| | TGAACGGTAAAGAATATAAATGCAAGGTTAGCAATAAAGCTTTGCCCGCCCCTATA |
| | GAGAAAACGATCAGTAAGGCGAAGGGGCAGCCTAGGGAACCCCAGGTATATACCTT |
| | GCCGCCAAGTCGAGATGAGCTGACGAAGAACCAAGTGAGTCTGACATGCCTCGTGA |
| | AGGGCTTCTATCCGAGCGATATCGCTGTCGAATGGGAGAGCAATGGGCAGCCTGAG |
| | AATAACTATAAAACAACGCCACCCGTCCTCGACTCCGATGGCTCATTCTTCCTGTA |
| | CAGTAAACTTACAGTAGATAAGAGTAGATGGCAGCAGGGTAACGTCTTTAGTTGCT |
| | CCGTGATGCACGAGGCATTGCACAATCATTACACTCAAAAATCTCTGTCCCTGAGT |
| | CCGGGCAAAGGCGGTTCAGATAGCTGGATGGAGGAGGTCATAAAGCTTTGTGGACG |
| | AGAACTCGTTCGCGCCCAGATAGCTATTTGTGGGAAATCAACCGGGGGTGGAGAAG |
| | GTGGCGGAGAAGGGGGAGGCGAAGGGCGCCAACTGTATTCTGCATTGGCTAATAAG |
| | TGCTGTCACGTAGGATGTACAAAAAGGTCTCTGGCGAGATTCTGC |
| 468 | GACAAGACGCACACTTGTCCACCTTGCCCTGCGCCGGAAGCTGCTGGAGGCCCCAG |
| | TGTCTTTTTGTTCCCGCCCAAACCGAAGGACACTTTGATGATAAGTCGCACGCCCG |
| | AGGTTACCTGTGTGGTTGTCGATGTCTCACACGAAGATCCGGAGGTGAAGTTTAAT |
| | TGGTATGTAGATGGCGTGGAGGTTCATAACGCCAAAACGAAACCCAGAGAAGAACA |
| | ATATAACAGTACATATCGAGTAGTATCCGTTCTCACTGTCCTGCATCAAGACTGGT |
| | TGAACGGGAAGGAATATAAGTGCAAGGTGAGCAATAAAGCACTCCCGGCCCCAATC |
| | GAAAAGACCATCAGCAAAGCGAAGGGGCAACCTCGAGAACCCCAGGTATATACGCT |
| | CCCCCCTAGTCGGGATGAACTTACTAAAAATCAGGTTAGCCTCACTTGCCTTGTTA |
| | AAGGGTTCTATCCCAGTGATATTGCCGTCGAATGGGAATCAAACGGGCAGCCGGAA |
| | AATAACTACAAGACAACCCCTCCTGTGCTCGATAGCGATGGCTCTTTTTTCCTCTA |
| | CAGCAAACTTACCGTTGATAAGAGCCGGTGGCAACAAGGTAATGTTTTCTCCTGCT |
| | CCGTTATGCATGAAGCACTCCATAACCATTATACCCAAAAAAGCCTGTCACTTAGT |
| | CCGGGTAAAGGAGGTAGTGATTCTTGGCAGGAGGAGGTAATCAAACTTTGTGGGAG |
| | GGAGCTGGTACGAGCTCAGATTGCTATATGTGGAAAAAGCACGGGCGGAGGAGAAG |
| | GAGGTGGCGAAGGCGGGGGTGAAGGTCGGCAACTCTACTCCGCTCTCGCTAATAAG |
| | TGCTGCCACGTCGGGTGTACGAAGCGCTCCCTGGCGCGATTCTGC |
| 469 | GATAAAACGCACACGTGTCCGCCCTGCCCAGCGCCTGAAGCCGCAGGCGGGCCGTC |
| | CGTCTTCCTCTTTCCTCCAAAACCCAAAGACACACTTATGATCAGTAGGACCCCAG |
| | AGGTAACCTGCGTCGTGGTCGACGTTTCCCATGAAGACCCAGAGGTCAAGTTCAAC |
| | TGGTACGTCGACGGTGTCGAAGTACATAATGCTAAAACGAAGCCTCGGGAAGAGCA |
| | GTACAACTCTACCTACCGCGTCGTTTCCGTACTCACCGTACTTCACCAGGACTGGC |
| | TTAACGGTAAAGAGTATAAATGCAAAGTATCTAATAAGGCTCTCGCCGCGCCGATT |
| | GAGAAGACAATTTCAAAGGCCAAGGGGCAGCCGCGGGAGCCCCAAGTGTATACCTT |
| | GCCCCCGTCCCGAGATGAGCTGACTAAAAACCAAGTAAGCTTGACTTGCTTGGTCA |
| | AAGGCTTCTACCCTTCCGATATAGCTGTCGAATGGGAGTCAAATGGCCAACCAGAG |
| | AACAATTATAAAACTACACCCCCGGTCTTGGATTCTGATGGCTCATTTTTTCTCTA |
| | TTCTAAACTGACCGTGGATAAGTCTCGCTGGCAGCAAGGTAACGTGTTCAGTTGCT |
| | CTGTTCTTCACGAAGCACTGCACAGTCATTACACTCAGAAGAGTCTTAGCCTGAGC |
| | CCTGGTAAAGGGGGTTCTGATTCCTGGCAGGAGGAAGTAATAAAACTCTGTGGCCG |
| | GGAGTTGGTACGGGCGCAGATTGCGATATGCGGTAAGAGCACCGGCGGAGGCGAAG |
| | GCGGTGGGGAAGGAGGAGGAGAAGGGAGACAACTCTATTCCGCATTGGCAAATAAG |
| | TGCTGCCACGTCGGGTGTACCAAACGATCCCTTGCACGGTTCTGT |
| 470 | GATAAGACCCATACGTGCCCCCCTTGCCCTGCGCCTGAGGCAGCGGGTGGCCCATC |
| | AGTCTTTTTGTTCCCGCCCAAGCCAAAGGACACCCTCATGATTAGTAGAACACCGG |
| | AGGTTACGTGCGTCGTAGTGGATGTCAGCCACGAGGATCCCGAGGTTAAGTTTAAC |
| | TGGTACGTTGATGGGGTTGAGGTCCATAATGCGAAGACTAAGCCGAGAGAGGAACA |
| | GTACAATTCCACGTATAGAGTTGTCTCTGTACTGACTGTGCTGCATCAAGATTGGC |
| | TTAACGGTAAGGAGTACAAGTGCAAAGTCTCTAATAAGGCTCTTCCTGCACCCATT |
| | GAGAAAACTATAAGCAAAGCAAAAGGTCAACCTCGCGAACCTCAGGTGTACACACT |
| | GCCACCCTCTAGGGACGAGCTTACCAAAAATCAAGTATCTCTTACCTGCCTTGTGA |
| | AAGGGTTTTATCCCTCAGATATTGCGGTTGAGTGGGAGTCTAACGGACAACCTGAG |
| | AACAACTATAAGACTACTCCCCCGGTGCTTGATTCAGACGGGAGTTTTTTTTTGTA |
| | TAGCAAACTTACCGTCGACAAAAGCCGGTGGCAACAGGGCAATGTATTCAGTTGTT |
| | CTGTAATGCATGAAGCTTTGCATAATCATTACACCCAAAAGAGTCTTTCCCTGTCT |
| | CCTGGAAAAGGGGGGTCAGACTCCTGGATGGAGGAGGTGATCAAACTGTGTGGGAG |
| | AGAGCTCGTCCGGGCTCAGATAGCTATATGCGGCAAGTCTACGGGTGGGGGAGAGG |
| | GCGGAGGAGAGGGCGGTGGAGAAGGAGGCGGCCAACTCTACAGCGCTCTGGCCAAT |
| | AAATGTTGTCATGTCGGGTGTACTAAGCGCTCACTGGCACGCTTTTGC |
| 471 | GACAAGACGCATACATGCCCGCCATGCCCGGCCCCCGAAGCTGCTGGGGGACCATC |
| | CGTATTCCTCTTCCCTCCCAAACCAAAAGACACGTTGATGATAAGTAGAACACCAG |
| | AGGTAACGTGCGTGGTTGTCGATGTTTCCCACGAAGATCCGGAGGTAAAATTCAAT |
| | TGGTATGTAGATGGGGTGGAAGTGCACAATGCCAAAACAAAGCCGCGAGAAGAACA |
| | ATACAATAGTACTTACCGGGTTGTGAGCGTGCTCACGGTGTTGCACCAAGACTGGC |
| | TCAACGGCAAGGAATACAAGTGCAAAGTATCTAATAAAGCTCTGCCTGCGCCGATA |
| | GAGAAGACCATCAGTAAGGCCAAAGGGCAGCCCCGAGAGCCGCAAGTTTACACTCT |
| | TCCTCCGAGCAGAGATGAATTGACCAAGAACCAAGTAAGTTTGACGTGCCTGGTGA |
| | AGGGCTTCTACCCCTCAGACATTGCGGTGGAGTGGGAAAGTAATGGTCAACCGGAA |
| | AACAACTACAAGACCACGCCGCCCGTCCTCGACTCCGATGGGTCTTTCTTTCTTTA |
| | TTCAAAGTTGACAGTAGATAAGTCAAGGTGGCAGCAAGGTAACGTGTTTAGTTGTA |
| | GTGTAATGCACGAGGCCCTGCATAATCATTATACCCAAAAGAGTTTGAGCCTCTCA |
| | CCAGGAAAAGGCGGATCAGACAGCTGGCAGGAGGAGGTAATTAAATTGTGTGGACG |
| | GGAGTTGGTCAGGGCGCAAATAGCCATCTGCGGTAAGAGCACGGGTGGAGGAGAGG |
| | GTGGAGGGGAAGGTGGGGGAGAAGGCGGCGGGCAGCTCTATTCTGCACTCGCCAAC |
| | AAGTGTTGTCACGTCGGATGCACAAAGAGATCTCTTGCTCGATTCTGC |
| 472 | GACAAAACACACACCTGTCCGCCTTGCCCGGCTCCTGAAGCCGCGGGTGGCCCTAG |
| | TGTGTTTTTGTTTCCGCCGAAACCTAAGGATACCCTCATGATAAGCCGGACGCCCG |
| | AGGTTACCTGTGTCGTGGTCGATGTTAGTCATGAGGATCCAGAAGTCAAGTTTAAT |
| | TGGTACGTCGACGGCGTTGAAGTCCACAATGCAAAAACTAAACCGCGAGAAGAACA |
| | GTACAACTCCACCTACAGAGTTGTCTCAGTTTTGACAGTTCTCCATCAGGATTGGC |
| | TCAATGGAAAGGAATATAAGTGCAAGGTCAGCAATAAAGCGCTTGCCGCCCCTATA |
| | GAGAAGACCATTAGCAAGGCGAAAGGACAGCCCCGCGAGCCCCAGGTCTATACGCT |
| | GCCTCCTAGCAGAGATGAGCTCACGAAAAATCAGGTCAGCTTGACATGCTTGGTGA |
| | AGGGCTTCTACCCCAGTGACATCGCAGTTGAATGGGAGAGCAACGGCCAACCTGAG |
| | AACAACTACAAAACAACGCCCCCGGTTCTTGACAGCGATGGGTCCTTCTTTCTTTA |
| | CTCTAAGCTTACAGTTGATAAAAGCAGGTGGCAGCAGGGGAATGTGTTCTCATGTT |
| | CCGTACTGCATGAGGCTCTGCATTCTCACTACACCCAAAAAAGCCTTAGCCTGAGC |
| | CCCGGTAAGGGAGGTAGTGACTCATGGCAAGAGGAAGTGATTAAGCTCTGCGGCCG |
| | GGAGTTGGTGAGAGCCCAAATCGCCATTTGCGGTAAAAGTACCGGAGGGGGCGAGG |
| | GAGGAGGCGAAGGTGGAGGTGAAGGAGGTGGACAGTTGTACTCAGCTCTTGCAAAT |
| | AAATGTTGTCATGTTGGTTGCACGAAAAGATCTCTTGCGAGGTTCTGT |
| 473 | GATAAGACGCATACTTGTCCACCGTGCCCCGCACCGGAAGCGGCTGGTGGTCCATC |
| | AGTTTTTCTGTTCCCACCGAAACCTAAGGACACGTTGATGATATCACGGACACCAG |
| | AGGTTACGTGCGTAGTGGTGGATGTGAGCCACGAGGATCCAGAAGTTAAATTTAAT |
| | TGGTACGTAGATGGAGTGGAGGTTCATAATGCGAAGACAAAGCCTCGCGAGGAACA |
| | GTATAATTCCACCTATCGCGTCGTATCTGTGCTTACGGTACTTCACCAAGACTGGT |
| | TGAACGGTAAGGAATATAAATGCAAGGTTTCCAATAAAGCACTTCCTGCGCCAATT |
| | GAGAAGACAATATCCAAAGCTAAAGGTCAACCCAGGGAACCGCAAGTCTACACTCT |
| | CCCCCCGTCTCGCGATGAATTGACGAAGAACCAGGTTAGTCTCACCTGCCTGGTCA |
| | AGGGGTTTTACCCCTCTGACATAGCTGTAGAATGGGAGTCTAATGGACAGCCAGAG |
| | AACAATTACAAAACGACCCCCCCGGTCCTCGATTCTGATGGGAGTTTTTTTCTTTA |
| | TTCAAAATTGACTGTCGATAAGTCAAGATGGCAACAGGGTAACGTATTTTCTTGCA |
| | GTGTTATGCATGAAGCATTGCACAACCACTATACACAAAAATCATTGAGTTTGAGT |
| | CCCGGTAAAGGGGGAAGCGACTCATGGATGGAAGAAGTAATCAAGCTGTGCGGGCG |
| | AGAGCTTGTGCGAGCTCAGATAGCAATCTGTGGTAAGTCTACAGGTGGAGAGGGTG |
| | GCGGTGAAGAAGGCGGGGGAGAGGGAGGCCAGCTTTATTCTGCCCTGGCTAACAAG |
| | TGCTGTCACGTTGGATGCACGAAGCGCTCCCTGGCCCGATTCTGC |
| 474 | GATAAGACGCATACTTGTCCCCCATGTCCCGCTCCGGAAGCCGCTGGCGGCCCCTC |
| | CGTTTTTCTGTTCCCGCCGAAACCGAAAGACACCCTGATGATATCACGCACTCCCG |
| | AGGTCACTTGCGTGGTAGTCGATGTTAGTCATGAAGATCCTGAGGTCAAATTCAAT |
| | TGGTATGTAGATGGCGTTGAGGTACACAACGCGAAGACAAAACCCCGAGAAGAACA |
| | GTATAACTCAACCTACCGCGTAGTTTCAGTTCTTACCGTACTGCACCAAGACTGGT |
| | TGAACGGTAAAGAGTACAAATGTAAAGTCAGCAATAAAGCTTTGCCAGCACCTATC |
| | GAAAAAACCATCAGTAAGGCCAAGGGTCAACCCAGGGAGCCGCAAGTGTACACTCT |
| | TCCCCCTAGCAGGGATGAATTGACCAAGAATCAGGTCTCTTTGACGTGCCTCGTTA |
| | AGGGTTTCTATCCCAGCGATATAGCCGTAGAATGGGAGTCTAACGGTCAGCCAGAA |
| | AATAACTATAAGACAACCCCGCCTGTTTTGGATTCCGACGGCTCTTTTTTTCTCTA |
| | CTCTAAGTTGACCGTTGATAAGAGCAGATGGCAGCAGGGAAACGTATTTTCTTGTT |
| | CCGTGATGCACGAAGCCCTGCACAATCACTATACGCAAAAGTCTCTGAGCTTGAGT |
| | CCGGGTAAAGGCGGTTCTGACTCCTGGCAGGAGGAAGTCATAAAACTCTGCGGAAG |
| | AGAGCTCGTAAGGGCGCAAATCGCTATTTGTGGTAAGAGCACCGGTGGGGAAGGAG |
| | GCGGTGAAGAGGGTGGCGGCGAGGGTGGGCAATTGTATTCCGCGCTTGCCAATAAA |
| | TGTTGTCACGTAGGCTGCACAAAGCGAAGTCTCGCTAGGTTCTGC |
| 475 | GACAAGACCCACACATGTCCCCCGTGTCCGGCACCAGAAGCAGCGGGGGGACCGTC |
| | AGTATTCTTGTTTCCACCGAAGCCCAAAGACACATTGATGATTTCACGAACTCCTG |
| | AAGTTACCTGTGTGGTTGTAGATGTATCACACGAAGACCCAGAAGTCAAATTCAAT |
| | TGGTATGTCGACGGGGTTGAAGTTCACAATGCGAAGACGAAGCCCCGGGAGGAACA |
| | GTACAACAGCACGTACAGGGTTGTGAGCGTTCTTACTGTATTGCACCAGGATTGGC |
| | TCAACGGCAAGGAGTATAAATGTAAAGTTTCTAATAAGGCTCTTCCTGCCCCAATT |
| | GAAAAGACGATATCTAAAGCGAAGGGCCAACCACGGGAACCTCAGGTGTACACACT |
| | TCCGCCTAGCAGGGATGAGTTGACCAAGAATCAAGTCTCTTTGACGTGCCTGGTCA |
| | AGGGGTTTTACCCATCAGATATCGCCGTCGAATGGGAGTCAAACGGACAACCCGAA |
| | AATAACTATAAAACTACTCCACCAGTTCTGGATAGCGACGGCTCATTTTTTCTGTA |
| | TTCAAAGCTCACTGTAGACAAGTCTAGGTGGCAGCAGGGTAATGTCTTCTCCTGCT |
| | CAGTAATGCATGAGGCTCTTCACAACCACTATACTCAAAAGAGCCTTTCCCTGTCA |
| | CCTGGCGGTGGAAGCGACTCATGGATGGAGGAGGTAATAAAGCTCTGCGGAAGAGA |
| | ACTGGTACGCGCACAAATCGCAATTTGTGGTAAGAGTACTGGCGGGGAAGGAGGTG |
| | GGGAAGAAGGGGGCGGTGAGGGCGGACAGCTCTATTCTGCACTTGCAAACAAATGT |
| | TGCCACGTGGGATGTACTAAGCGAAGCCTTGCAAGATTCTGC |
| 476 | GATAAAACCCACACATGCCCTCCATGCCCTGCTCCAGAGGCCGCCGGTGGGCCATC |
| | AGTTTTCTTGTTTCCGCCTAAACCAAAGGACACGCTTATGATCTCCAGGACCCCCG |
| | AAGTTACGTGTGTGGTGGTTGATGTTAGTCACGAGGACCCGGAAGTCAAGTTCAAC |
| | TGGTACGTTGATGGTGTAGAGGTGCACAATGCAAAGACGAAGCCACGCGAAGAACA |
| | ATACAACAGCACATATCGAGTTGTGAGCGTACTCACGGTACTGCATCAGGACTGGC |
| | TGAACGGTAAAGAATACAAATGTAAAGTCTCCAATAAGGCACTTCCTGCGCCGATA |
| | GAAAAAACGATCAGTAAGGCCAAGGGCCAACCCCGAGAACCACAGGTATATACGCT |
| | CCCACCGTCACGAGACGAGTTGACAAAAAATCAGGTCTCCCTGACTTGCCTCGTGA |
| | AAGGTTTTTATCCCTCAGATATTGCTGTTGAGTGGGAAAGCAATGGGCAGCCAGAG |
| | AATAATTATAAGACGACTCCTCCGGTTTTGGATTCCGACGGTAGTTTTTTCTTGTA |
| | TAGTAAGCTTACTGTAGACAAGTCAAGATGGCAACAAGGTAATGTGTTCTCTTGCT |
| | CAGTTATGCATGAAGCTCTTCATAACCATTACACGCAAAAGAGTCTCAGTCTGAGC |
| | CCCGGTGGCGGTAGCGACAGTTGGCAGGAAGAGGTGATTAAGTTGTGCGGTCGCGA |
| | GCTCGTTCGGGCCCAAATTGCAATCTGCGGAAAATCTACGGGCGGAGAGGGCGGGG |
| | GTGAGGAGGGTGGGGGTGAAGGTGGGCAGCTCTATAGCGCCCTTGCGAATAAATGT |
| | TGTCACGTCGGATGCACAAAGAGGTCCCTCGCCAGGTTCTGC |
| 477 | GATAAGACCCACACTTGCCCCCCTTGCCCTGCCCCCGAAGCGGCCGGAGGTCCTTC |
| | AGTATTTTTGTTTCCACCGAAACCCAAAGATACTTTGATGATATCAAGAACTCCTG |
| | AAGTCACCTGCGTGGTAGTTGACGTATCTCATGAGGATCCCGAGGTGAAGTTCAAT |
| | TGGTACGTCGATGGCGTCGAGGTTCATAACGCTAAGACTAAGCCGAGGGAAGAGCA |
| | ATATAATTCCACTTATAGGGTGGTGTCCGTCTTGACTGTTTTGCACCAGGATTGGT |
| | TGAACGGGAAAGAGTACAAATGTAAGGTGAGTAATAAAGCTTTGGCTGCTCCCATC |
| | GAAAAGACAATAAGCAAGGCCAAGGGGCAACCTCGGGAGCCGCAGGTGTACACCCT |
| | TCCTCCCAGTAGAGACGAACTGACAAAAAACCAGGTGTCCCTGACCTGCCTTGTGA |
| | AGGGGTTTTACCCGAGCGACATAGCGGTTGAATGGGAGAGCAACGGGCAACCCGAG |
| | AACAACTACAAAACTACACCGCCTGTCCTGGACTCCGATGGAAGCTTCTTCCTCTA |
| | CTCCAAACTGACCGTGGACAAAAGCAGATGGCAACAAGGAAACGTATTCTCATGCT |
| | CAGTAATGCACGAAGCATTGCACAATCACTACACCCAAAAGTCCCTCTCACTCTCC |
| | CCTGGTAAGGGCGGATCAGACTCATGGCAAGAGGAGGTAATTAAGTTGTGCGGGAG |
| | GGAGCTCGTCCGCGCGCAAATAGCCATTTGTGGCAAGTCCACTGGAGGAGGCGAGG |
| | GTGGAGGAGAGGGTGGTGGGGAGGGCAGGCAACTCTACAGTGCGCTCGCCAATAAA |
| | TGCTGCCATGTTGGGTGCACGAAGCGCAGTCTCGCACAATTCTGC |
| 478 | GATAAGACCCACACGTGTCCTCCATGTCCGGCACCGGAGGCTGCTGGCGGGCCTTC |
| | TGTATTCCTCTTCCCACCCAAGCCAAAAGACACATTGATGATATCAAGGACGCCGG |
| | AAGTCACCTGTGTTGTTGTGGACGTTTCCCATGAAGACCCAGAGGTAAAATTCAAT |
| | TGGTATGTGGACGGCGTAGAGGTTCACAACGCCAAAACCAAACCCCGAGAGGAACA |
| | GTATAATAGCACATATCGAGTAGTATCTGTTCTCACAGTGCTCCATCAAGACTGGC |
| | TTAATGGTAAAGAGTATAAATGCAAAGTTTCCAATAAAGCCCTCGCTGCACCGATC |
| | GAGAAGACAATCAGTAAAGCGAAGGGCCAGCCTCGGGAACCGCAGGTGTATACTCT |
| | TCCACCCTCAAGAGACGAGCTCACTAAAAACCAAGTTTCATTGACATGCCTCGTCA |
| | AAGGTTTCTACCCATCAGACATCGCGGTCGAATGGGAAAGTAATGGGCAGCCGGAA |
| | AACAACTATAAAACGACGCCGCCCGTCTTGGATTCTGATGGTTCATTTTTTCTTTA |
| | CTCTAAATTGACCGTCGATAAAAGTAGGTGGCAACAAGGAAATGTTTTTTCCTGCT |
| | CCGTCCTGCATGAAGCGTTGCACAGTCACTATACCCAGAAGAGTCTTTCTTTGTCA |
| | CCCGGAAAAGGCGGTTCAGATTCATGGCAGGAAGAAGTAATTAAACTCTGTGGCCG |
| | CGAGCTTGTTAGGGCGCAGATAGCCATATGTGGTAAAAGCACCGGAGGAGGTGAAG |
| | GCGGAGGCGAAGGAGGTGGGGAAGGAAGACAATTGTATTCTGCACTTGCAAATAAA |
| | TGCTGTCATGTGGGGTGCACGAAACGCAGTCTTGCACAATTTTGT |
| 479 | GACAAAACCCATACCTGCCCCCCTTGCCCTGCACCAGAAGCGGCGGGAGGACCTAG |
| | CGTTTTTCTTTTTCCTCCGAAACCGAAAGATACCCTCATGATATCAAGAACACCTG |
| | AGGTTACTTGCGTTGTCGTGGACGTGAGTCACGAAGACCCCGAGGTGAAGTTCAAC |
| | TGGTATGTAGATGGAGTGGAGGTCCATAATGCAAAAACGAAACCGAGAGAAGAACA |
| | ATACAACTCTACATATCGAGTCGTGTCAGTACTCACGGTTTTGCATCAAGATTGGC |
| | TGAACGGTAAGGAGTACAAGTGTAAGGTTAGCAACAAGGCTCTCGCGGCGCCGATA |
| | GAAAAGACTATAAGTAAAGCAAAAGGCCAGCCCAGAGAACCTCAAGTTTACACTCT |
| | GCCTCCCAGCAGAGATGAACTGACTAAAAATCAGGTTTCATTGACCTGTCTCGTCA |
| | AGGGTTTTTATCCAAGCGACATAGCAGTTGAATGGGAAAGCAACGGTCAACCAGAA |
| | AATAATTACAAAACCACTCCACCAGTCTTGGACTCTGACGGATCCTTCTTTCTCTA |
| | TTCAAAATTGACGGTGGATAAATCTAGGTGGCAGCAAGGCAACGTCTTCTCTTGTA |
| | GCGTTATGCATGAGGCGCTGCACAACCACTACACACAAAAGTCTCTTAGTTTGAGC |
| | CCGGGCGGCGGAAGCGACTCTTGGCAAGAGGAAGTGATAAAACTCTGTGGTCGAGA |
| | ATTGGTACGCGCGCAGATCGCTATCTGCGGCAAGTCCACAGGGGGAGGGGAAGGTG |
| | GCGGGGAAGGTGGTGGCGAGGGCAGGCAGTTGTATAGTGCACTTGCCAACAAGTGC |
| | TGCCATGTGGGGTGCACCAAGCGCAGTTTGGCACGGTTCTGC |
| 480 | GATAAAACTCACACTTGTCCCCCGTGTCCGGCACCAGAAGCCGCAGGAGGGCCATC |
| | TGTCTTTCTTTTTCCCCCAAAACCCAAGGATACACTGATGATCTCCCGCACTCCCG |
| | AAGTTACTTGTGTCGTAGTAGACGTTTCTCACGAGGACCCAGAGGTGAAATTCAAT |
| | TGGTATGTTGACGGAGTAGAGGTGCATAATGCCAAGACAAAGCCCCGAGAGGAACA |
| | ATACAATTCAACCTACAGAGTAGTGTCCGTTCTTACGGTTCTCCATCAGGATTGGC |
| | TCAACGGTAAGGAATATAAGTGCAAGGTAAGCAACAAAGCGCTGGCCGCACCCATT |
| | GAGAAAACCATTTCAAAAGCTAAAGGCCAACCCCGCGAACCACAAGTTTATACTCT |
| | CCCCCCAAGTCGCGATGAACTTACAAAAAATCAAGTCTCATTGACGTGCTTGGTCA |
| | AAGGCTTCTACCCGAGCGATATCGCTGTTGAATGGGAGTCTAATGGACAACCGGAA |
| | AATAACTATAAAACTACACCCCCAGTCCTCGATTCAGACGGCAGCTTCTTCCTGTA |
| | TTCAAAACTGACGGTTGACAAATCACGCTGGCAACAGGGTAACGTTTTTTCCTGTA |
| | GCGTTCTTCATGAAGCCTTGCACAGTCACTACACCCAGAAGTCCCTTAGCTTGTCA |
| | CCTGGCGGGGGTTCAGACTCTTGGCAGGAGGAGGTAATCAAACTGTGCGGAAGAGA |
| | ACTGGTGAGGGCTCAGATTGCAATTTGTGGGAAGAGCACGGGTGGCGGTGAAGGAG |
| | GTGGCGAGGGCGGAGGAGAGGGGAGGCAACTCTACAGTGCGTTGGCTAATAAATGC |
| | TGTCACGTCGGCTGTACTAAGAGAAGCCTCGCCAGATTTTGC |
| 481 | GACAAGACGCATACTTGCCCTCCGTGCCCTGCACCAGAAGCCGCTGGTGGCCCATC |
| | TGTGTTTTTGTTCCCCCCTAAGCCAAAAGACACATTGATGATTTCACGAACTCCAG |
| | AAGTGACTTGCGTAGTTGTTGACGTATCACACGAAGACCCCGAGGTTAAATTTAAT |
| | TGGTATGTGGACGGGGTCGAGGTGCATAACGCCAAAACCAAACCCCGGGAGGAACA |
| | ATATAACTCTACGTATCGGGTCGTATCTGTGTTGACCGTCCTTCACCAAGATTGGT |
| | TGAACGGCAAGGAATATAAGTGTAAAGTGTCTAATAAAGCATTGGCTGCCCCGATA |
| | GAAAAGACGATCTCTAAAGCCAAGGGCCAACCCAGAGAGCCTCAAGTATATACTCT |
| | CCCACCGAGTCGAGATGAGCTCACTAAGAACCAGGTGTCACTCACGTGTCTGGTTA |
| | AAGGATTTTACCCTAGTGATATAGCCGTCGAGTGGGAATCAAATGGGCAGCCGGAG |
| | AATAACTATAAGACCACGCCTCCAGTTCTCGATTCCGATGGTAGCTTTTTCCTTTA |
| | CTCTAAACTTACGGTCGACAAGTCCAGGTGGCAACAGGGCAATGTATTTTCTTGCT |
| | CCGTCATGCACGAGGCTTTGCACAACCATTACACGCAAAAGTCACTGTCCCTGTCT |
| | CCTGGAGGCGGTTCTGACAGTTGGCAGGAGGAGGTAATCAAATTGTGTGGGCGGGA |
| | GTTGGTTAGGGCGCAAATTGCTATTTGCGGCAAAAGTACTGGGGGCGGTGAAGGCG |
| | GAGGCGAGGGAGGAGGAGAAGGTCGACAACTGTATTCTGCCTTGGCGAACAAATGC |
| | TGTCACGTCGGCTGTACGAAACGGTCTTTGGCCCAGTTTTGT |
| 482 | GATAAGACACACACTTGTCCGCCATGCCCTGCGCCGGAAGCGGCGGGAGGACCGTC |
| | CGTTTTCCTGTTCCCTCCCAAACCCAAAGACACGTTGATGATTAGTCGCACGCCAG |
| | AAGTTACGTGCGTTGTCGTAGATGTATCCCACGAAGACCCCGAGGTGAAGTTCAAT |
| | TGGTATGTAGATGGGGTGGAGGTCCATAACGCTAAGACCAAACCACGCGAGGAACA |
| | ATATAATTCTACGTACCGCGTAGTGAGCGTTCTCACAGTTCTTCACCAGGATTGGC |
| | TTAACGGCAAGGAGTATAAGTGTAAGGTGTCTAATAAGGCCTTGGCTGCCCCGATC |
| | GAAAAAACGATAAGTAAAGCAAAGGGTCAACCTAGAGAACCCCAAGTGTACACTCT |
| | CCCGCCATCACGGGATGAATTGACTAAGAACCAAGTGTCACTCACGTGTCTTGTAA |
| | AGGGCTTCTACCCATCCGATATAGCCGTTGAGTGGGAATCCAATGGTCAGCCAGAG |
| | AACAACTATAAGACAACTCCGCCCGTACTTGATAGTGACGGTTCCTTTTTCCTTTA |
| | CAGTAAATTGACGGTAGATAAGTCTCGCTGGCAGCAAGGAAACGTCTTTTCTTGTT |
| | CAGTGCTTCATGAGGCGCTTCACTCACACTATACTCAGAAGAGTTTGAGTTTGTCT |
| | CCAGGTGGAGGCAGCGACTCATGGCAAGAGGAAGTAATCAAACTGTGTGGTCGCGA |
| | ATTGGTACGAGCACAGATCGCGATCTGCGGGAAATCAACAGGTGGCGGCGAAGGCG |
| | GCGGGGAAGGCGGCGGCGAAGGTAGGCAACTTTACTCAGCCCTTGCGAACAAATGT |
| | TGCCACGTAGGCTGTACTAAGAGAAGTCTCGCCCAGTTTTGC |
| 483 | GACAAGACTCATACCTGCCCCCCTTGTCCAGCACCAGAAGCAGCTGGCGGGCCAAG |
| | CGTGTTCCTGTTTCCACCTAAGCCCAAAGATACGTTGATGATCAGCCGCACCCCGG |
| | AAGTAACCTGTGTAGTAGTAGATGTGTCCCACGAAGACCCCGAAGTAAAGTTTAAT |
| | TGGTACGTCGATGGTGTCGAAGTACATAACGCTAAAACGAAGCCCCGAGAAGAGCA |
| | GTACAACAGTACTTACAGAGTAGTTTCTGTTCTTACAGTGCTGCATCAGGATTGGC |
| | TGAACGGGAAGGAGTATAAATGTAAAGTCTCAAACAAGGCACTTGCGGCACCAATA |
| | GAGAAGACAATATCTAAGGCCAAAGGGCAGCCTAGAGAGCCACAAGTATATACGCT |
| | GCCCCCCAGCAGGGACGAGCTGACAAAGAACCAAGTGTCACTGACCTGCCTTGTTA |
| | AGGGCTTCTATCCGAGTGATATTGCTGTTGAATGGGAAAGTAACGGACAGCCGGAG |
| | AACAACTATAAAACTACTCCACCCGTGTTGGATAGTGACGGTAGCTTTTTTCTGTA |
| | CTCCAAGTTGACGGTAGACAAAAGTCGGTGGCAGCAGGGGAACGTATTTTCTTGTT |
| | CTGTCATGCACGAAGCTCTTCACAATCACTATACGCAGAAGTCCCTCTCTCTCTCT |
| | CCTGGGAAGGGTGGTTCCGACAGCTGGCAGGAGGAGGTCATTAAACTGTGTGGTAG |
| | AGAGCTGGTACGGGCTCAAATTGCAATTTGTGGTAAGAGTACTGGCGGTGGCGAGG |
| | AAGGGGGTGGGGAGGAGGGCGGAGGTAGGCAGCTCTACTCTGCTCTCGCCAACAAG |
| | TGTTGTCACGTCGGGTGTACTAAAAGATCACTTGCCCGCTTTTGT |
| 484 | GACAAAACACATACATGCCCGCCGTGTCCGGCGCCTGAAGCAGCAGGAGGCCCCAG |
| | TGTATTCCTTTTCCCTCCAAAGCCAAAAGATACGTTGATGATATCTAGGACACCTG |
| | AGGTTACCTGCGTCGTAGTGGACGTATCCCACGAAGACCCAGAAGTCAAGTTTAAC |
| | TGGTATGTGGACGGAGTGGAGGTACACAATGCAAAGACAAAGCCGCGAGAGGAACA |
| | ATATAATTCCACCTATAGAGTCGTGTCAGTCCTTACGGTCTTGCACCAGGACTGGC |
| | TCAATGGTAAGGAGTATAAGTGCAAAGTATCAAACAAAGCTCTCGCAGCGCCCATC |
| | GAAAAGACCATCAGCAAAGCTAAGGGCCAGCCAAGAGAGCCTCAAGTGTACACGTT |
| | GCCGCCTTCAAGGGACGAGCTCACTAAAAATCAGGTATCACTTACGTGTCTTGTCA |
| | AAGGGTTTTATCCTTCCGACATCGCGGTTGAATGGGAGAGCAATGGACAGCCGGAG |
| | AATAATTATAAAACGACGCCGCCGGTCCTTGACAGCGATGGTTCATTTTTCCTTTA |
| | CTCAAAGCTGACGGTTGATAAGTCTAGGTGGCAGCAGGGGAACGTCTTTTCCTGTA |
| | GTGTACTTCATGAGGCGCTCCATTCTCATTACACTCAGAAGTCACTGAGCCTTTCA |
| | CCCGGCAAAGGTGGATCAGACTCCTGGCAAGAAGAGGTAATCAAACTCTGTGGGAG |
| | GGAACTCGTTCGAGCCCAGATTGCAATCTGTGGGAAAAGCACAGGCGGAGGGGAAG |
| | AAGGGGGTGGCGAAGAAGGTGGGGGCAGGCAGCTCTATTCAGCTCTTGCCAACAAA |
| | TGCTGTCATGTAGGCTGCACAAAGCGATCACTGGCGAGATTCTGT |
| 485 | GATAAAACTCATACTTGCCCACCCTGCCCGGCTCCCGAGGCAGCAGGTGGACCCTC |
| | AGTATTTTTGTTCCCTCCGAAACCTAAAGATACACTTATGATTAGCCGGACCCCTG |
| | AGGTAACGTGTGTGGTGGTTGACGTAAGTCATGAAGATCCAGAAGTAAAGTTTAAC |
| | TGGTACGTAGACGGTGTGGAGGTACATAATGCGAAGACAAAACCACGAGAGGAACA |
| | GTATAACTCTACCTACCGCGTAGTAAGCGTACTTACTGTGCTCCACCAAGACTGGC |
| | TTAACGGGAAAGAGTATAAGTGTAAAGTCAGTAATAAAGCACTGGCCGCCCCGATC |
| | GAAAAAACAATCAGCAAGGCCAAAGGACAACCAAGGGAGCCTCAGGTCTATACTCT |
| | TCCCCCGAGTAGGGATGAGCTTACCAAGAACCAGGTGTCTCTGACATGCCTTGTCA |
| | AGGGATTTTACCCGAGTGACATAGCCGTAGAATGGGAGTCAAACGGCCAACCTGAA |
| | AACAACTATAAGACCACGCCTCCCGTACTCGACTCAGATGGAAGCTTTTTCCTCTA |
| | TAGCAAGCTGACCGTCGACAAAAGTAGGTGGCAACAGGGAAACGTCTTTAGTTGTT |
| | CCGTCATGCACGAAGCTTTGCATAACCATTACACCCAGAAGAGTCTTTCCCTTTCC |
| | CCTGGCAAGGGGGGCTCCGACTCCTGGCAAGAGGAAGTAATCAAACTGTGTGGGCG |
| | CGAGCTTGTCCGCGCGCAAATAGCCATTTGCGGAAAAAGTACTGGAGGAGGAGAGG |
| | AAGGCGGCGGCGAGGAAGGTGGGGGCAGGCAGCTGTACAGTGCCTTGGCTAACAAG |
| | TGCTGCCATGTCGGCTGTACGAAAAGGTCTCTTGCTCAATTCTGT |
| 486 | GATAAGACACATACCTGTCCACCCTGCCCAGCACCTGAAGCTGCAGGCGGCCCCAG |
| | CGTATTCCTGTTTCCTCCGAAGCCGAAAGACACACTTATGATTTCCCGGACGCCTG |
| | AGGTAACTTGCGTCGTAGTAGATGTGTCTCACGAAGACCCCGAGGTGAAATTCAAC |
| | TGGTACGTTGATGGTGTGGAAGTTCATAATGCGAAAACTAAACCACGAGAGGAGCA |
| | ATATAACTCAACTTATAGAGTTGTGAGCGTCTTGACGGTACTGCACCAGGACTGGC |
| | TGAATGGCAAAGAGTACAAATGCAAAGTCTCAAATAAGGCGTTGGCGGCTCCCATA |
| | GAGAAAACTATCAGCAAAGCCAAGGGTCAACCTCGGGAGCCACAAGTGTATACTCT |
| | TCCGCCTAGTCGCGACGAGCTCACAAAGAATCAGGTGAGTCTTACTTGTTTGGTTA |
| | AGGGTTTCTACCCCAGTGACATTGCGGTCGAGTGGGAAAGTAACGGACAGCCTGAA |
| | AACAACTATAAAACAACGCCTCCAGTACTCGATTCAGATGGTTCATTCTTTCTTTA |
| | TTCCAAACTCACAGTCGACAAGAGTAGATGGCAACAAGGGAACGTGTTTAGCTGTA |
| | GCGTACTCCATGAGGCACTCCACTCTCACTATACCCAAAAGTCTCTCAGCTTGTCA |
| | CCCGGAAAAGGCGGTTCTGACAGTTGGCAAGAGGAAGTGATTAAATTGTGTGGGCG |
| | GGAACTTGTGAGGGCTCAAATCGCGATTTGCGGCAAGTCCACTGGTGGCGGCGAGG |
| | AAGGAGGAGGTGAAGAAGGAGGAGGTAGGCAACTGTATTCAGCGTTGGCGAATAAA |
| | TGCTGCCATGTTGGATGTACTAAACGGAGCCTTGCTCAGTTCTGC |
| 487 | GATAAAACGCATACTTGCCCTCCTTGCCCGGCACCTGAAGCTGCCGGAGGTCCTTC |
| | CGTGTTCCTGTTCCCACCTAAGCCAAAAGACACACTTATGATTTCTCGCACACCAG |
| | AAGTAACGTGCGTCGTAGTTGACGTCTCCCATGAAGACCCGGAGGTAAAATTTAAT |
| | TGGTACGTCGACGGGGTAGAAGTTCATAACGCAAAGACTAAACCACGAGAAGAGCA |
| | ATACAACTCTACATACAGAGTAGTAAGCGTTCTCACCGTTCTTCATCAAGATTGGC |
| | TCAACGGAAAGGAGTATAAGTGTAAGGTGTCCAATAAAGCGTTGGCCGCACCAATC |
| | GAAAAGACCATAAGCAAAGCCAAAGGCCAACCCCGCGAACCGCAGGTGTACACACT |
| | TCCCCCGTCCAGGGATGAATTGACAAAAAACCAAGTTTCCCTCACGTGTCTCGTCA |
| | AGGGATTCTACCCGAGTGATATCGCAGTTGAATGGGAAAGCAATGGTCAGCCCGAG |
| | AATAACTACAAGACTACTCCCCCTGTGTTGGACTCAGACGGCTCATTCTTCCTCTA |
| | CAGTAAGTTGACTGTGGACAAAAGTCGGTGGCAGCAAGGCAATGTCTTCAGTTGTA |
| | GTGTAATGCATGAAGCACTCCACAATCATTACACCCAAAAATCCCTGAGCCTGTCC |
| | CCGGGCGGAGGTTCAGATTCATGGCAGGAGGAAGTTATAAAACTGTGCGGGCGCGA |
| | GTTGGTGAGGGCGCAGATCGCAATCTGTGGAAAGAGTACGGGAGGTGGCGAAGAGG |
| | GTGGTGGAGAAGAGGGAGGAGGTCGACAACTGTATTCCGCGCTCGCGAACAAGTGT |
| | TGCCACGTTGGCTGCACCAAACGAAGCCTGGCTCGATTTTGC |
| 488 | GACAAGACACACACTTGTCCACCTTGCCCGGCTCCCGAGGCGGCAGGAGGACCAAG |
| | CGTTTTTCTGTTCCCTCCCAAACCAAAGGATACGCTTATGATCTCTCGAACGCCGG |
| | AAGTTACTTGCGTAGTAGTTGATGTCTCCCATGAAGATCCCGAAGTGAAGTTCAAC |
| | TGGTATGTAGATGGTGTGGAAGTTCATAACGCGAAAACCAAACCACGCGAAGAACA |
| | GTATAACAGTACTTATCGGGTTGTTTCAGTACTCACGGTGCTCCATCAAGACTGGC |
| | TTAATGGAAAGGAGTATAAATGTAAGGTAAGTAACAAGGCATTGGCGGCTCCCATC |
| | GAGAAGACAATCTCCAAAGCAAAAGGGCAACCACGGGAGCCTCAGGTGTATACGTT |
| | GCCGCCCAGCAGAGATGAACTTACTAAGAATCAGGTGAGTCTCACTTGTCTCGTCA |
| | AGGGCTTCTATCCCAGCGATATAGCCGTAGAATGGGAGAGTAACGGTCAGCCGGAG |
| | AACAACTACAAAACAACCCCGCCTGTTTTGGACTCCGATGGGAGTTTTTTTCTCTA |
| | CAGCAAACTCACGGTAGACAAAAGCAGGTGGCAGCAGGGCAATGTTTTCAGTTGCT |
| | CTGTTCTCCACGAAGCCCTCCACTCCCACTATACTCAGAAGTCTCTGAGTCTCTCA |
| | CCAGGGGGAGGTAGCGATAGCTGGCAGGAGGAAGTGATCAAGTTGTGCGGGCGCGA |
| | ACTCGTGCGGGCACAAATTGCTATATGCGGTAAAAGTACGGGAGGTGGAGAGGAGG |
| | GTGGAGGTGAAGAAGGCGGTGGTAGACAATTGTATAGTGCGCTCGCCAACAAGTGT |
| | TGTCATGTCGGGTGTACGAAACGGTCCTTGGCGCGGTTTTGC |
| 489 | GACAAGACACATACTTGTCCACCATGTCCCGCCCCAGAAGCTGCGGGAGGACCATC |
| | AGTTTTTTTGTTCCCCCCGAAACCGAAGGATACCCTCATGATAAGTCGAACGCCCG |
| | AAGTCACTTGCGTGGTGGTTGATGTTAGCCACGAGGACCCAGAAGTGAAGTTCAAC |
| | TGGTACGTGGACGGGGTCGAAGTTCATAATGCGAAAACAAAGCCTCGCGAGGAACA |
| | GTACAACTCTACATACAGGGTTGTGTCTGTTTTGACAGTCTTGCACCAAGATTGGC |
| | TCAACGGGAAGGAATATAAGTGTAAGGTAAGCAATAAAGCACTGGCGGCCCCGATC |
| | GAAAAAACGATATCCAAGGCCAAGGGCCAGCCCCGAGAGCCTCAGGTATATACTCT |
| | GCCGCCAAGCCGGGATGAACTGACTAAAAACCAGGTCTCTTTGACTTGTCTTGTCA |
| | AGGGATTTTACCCAAGTGACATTGCGGTAGAGTGGGAAAGCAACGGTCAACCAGAA |
| | AACAATTACAAGACGACACCGCCGGTACTCGACTCAGATGGATCCTTTTTCCTGTA |
| | TAGCAAGCTGACAGTGGACAAGTCCCGGTGGCAGCAAGGGAACGTATTTTCATGCA |
| | GCGTGATGCATGAGGCTCTTCACAACCATTACACACAGAAAAGTCTGTCATTGAGC |
| | CCTGGCGGCGGGAGCGATTCTTGGCAAGAAGAAGTTATAAAACTTTGCGGTCGAGA |
| | GCTGGTTCGGGCACAAATTGCTATCTGCGGAAAATCTACAGGAGGAGGCGAGGAGG |
| | GAGGGGGCGAAGAAGGCGGGGGGAGACAGTTGTACAGTGCGCTCGCTAACAAGTGT |
| | TGCCACGTCGGTTGCACAAAGAGATCCCTGGCTCAATTCTGT |
| 490 | GATAAAACTCACACCTGTCCCCCGTGTCCCGCACCAGAAGCGGCCGGTGGTCCCTC |
| | CGTTTTTCTCTTCCCTCCTAAACCTAAGGACACACTTATGATTAGCAGAACTCCAG |
| | AAGTTACGTGCGTAGTCGTTGACGTTAGTCATGAAGATCCTGAGGTTAAGTTCAAC |
| | TGGTACGTAGACGGAGTAGAGGTCCACAACGCCAAGACGAAACCCCGAGAAGAGCA |
| | GTATAATTCTACCTATCGAGTTGTTTCAGTATTGACGGTGCTTCACCAAGATTGGC |
| | TGAATGGCAAAGAGTATAAGTGCAAGGTAAGCAACAAAGCACTCGCGGCTCCTATC |
| | GAGAAAACTATTTCCAAAGCTAAGGGCCAGCCTCGCGAACCACAAGTCTATACCCT |
| | GCCACCGAGTCGGGACGAACTCACCAAGAACCAAGTGTCTCTTACTTGCCTCGTTA |
| | AAGGTTTTTATCCCAGCGACATAGCCGTCGAATGGGAGTCCAATGGCCAACCTGAG |
| | AACAACTATAAAACTACCCCTCCTGTACTTGATAGCGACGGAAGTTTTTTCCTCTA |
| | TTCAAAACTCACAGTTGATAAGTCTCGATGGCAACAGGGCAACGTCTTCTCTTGCA |
| | GTGTGTTGCATGAAGCTCTGCACTCTCATTACACACAGAAGAGTTTGTCTCTCAGT |
| | CCAGGTGGCGGCTCAGATAGCTGGCAGGAAGAAGTAATCAAGTTGTGCGGCAGGGA |
| | ACTGGTAAGGGCACAGATAGCCATTTGTGGAAAATCTACGGGTGGCGGTGAGGAAG |
| | GCGGCGGAGAAGAAGGGGGAGGTCGGCAGCTGTATAGTGCACTCGCAAACAAGTGC |
| | TGCCATGTCGGGTGCACCAAGCGATCCCTTGCCCAGTTTTGC |
| 491 | GATAAGACGCACACATGCCCACCCTGTCCTGCGCCTGAAGCCGCGGGGGGACCCAG |
| | CGTTTTTCTCTTCCCGCCGAAACCGAAAGACACACTTATGATCAGCCGGACTCCCG |
| | AGGTTACCTGCGTGGTGGTAGATGTATCTCACGAGGATCCCGAGGTCAAATTCAAC |
| | TGGTACGTTGATGGGGTTGAAGTTCATAATGCCAAAACGAAGCCAAGAGAAGAGCA |
| | GTATAACTCCACATATAGAGTTGTTTCCGTCTTGACTGTTCTTCACCAAGATTGGC |
| | TGAATGGGAAGGAGTACAAATGTAAAGTTAGCAACAAGGCACTCGCCGCTCCCATT |
| | GAAAAAACTATAAGCAAAGCTAAGGGCCAACCGCGCGAACCACAGGTCTACACGTT |
| | GCCGCCCTCTAGGGACGAACTCACGAAGAATCAGGTTTCCCTTACCTGCCTCGTTA |
| | AAGGATTCTACCCCTCTGACATAGCGGTTGAATGGGAGAGCAACGGTCAGCCTGAG |
| | AACAACTACAAAACGACGCCTCCGGTGTTGGATTCCGACGGTAGTTTTTTCCTCTA |
| | TAGTAAGCTGACAGTGGATAAATCTCGGTGGCAGCAAGGGAATGTATTCTCCTGTT |
| | CAGTCCTGCATGAAGCCCTCCACTCCCATTATACACAGAAATCTCTTTCTCTGAGT |
| | CCCGGTAAAGGTGGGAGTGACTCTTGGCAGGAAGAGGTAATTAAGTTGTGTGGAAG |
| | GGAGCTGGTAAGAGCACAGATTGCCATCTGTGGCAAATCCACGGGCGGCGAAGGTG |
| | AGGGGGGTGAGGGGGAAGGGGGGTCCAGACAACTGTATTCTGCTCTGGCGAATAAG |
| | TGTTGCCATGTAGGGTGCACTAAACGGTCCTTGGCGCAGTTCTGT |
| 492 | GATAAAACTCATACGTGCCCACCTTGCCCCGCACCGGAGGCTGCTGGAGGACCCTC |
| | TGTCTTCCTGTTCCCGCCGAAGCCTAAAGACACATTGATGATCAGTCGAACACCGG |
| | AAGTCACCTGTGTAGTGGTTGATGTGAGCCATGAGGACCCTGAAGTAAAATTTAAC |
| | TGGTATGTTGATGGCGTAGAAGTACACAACGCGAAGACTAAACCAAGGGAAGAGCA |
| | ATACAACTCTACCTATAGGGTCGTTAGCGTACTGACTGTGCTTCACCAAGACTGGC |
| | TTAACGGGAAGGAGTACAAGTGCAAAGTGAGCAATAAGGCCCTCGCCGCGCCTATC |
| | GAGAAAACCATTTCCAAAGCCAAGGGTCAACCAAGGGAGCCTCAGGTTTACACCCT |
| | GCCCCCTTCAAGGGATGAGTTGACAAAAAACCAGGTAAGTCTGACGTGTCTCGTTA |
| | AGGGATTCTACCCGTCAGATATCGCGGTAGAGTGGGAGAGCAACGGTCAGCCAGAA |
| | AATAATTACAAAACAACACCTCCAGTTTTGGACTCTGATGGGAGTTTTTTTCTTTA |
| | TTCTAAGTTGACAGTGGATAAGTCACGCTGGCAACAGGGGAACGTATTTAGCTGCT |
| | CAGTACTTCATGAAGCGTTGCATTCTCACTACACACAGAAGAGCCTCTCCTTGAGT |
| | CCCGGAGGTGGCTCTGATTCTTGGCAGGAGGAGGTAATAAAACTTTGTGGTAGAGA |
| | ACTGGTTCGCGCTCAGATAGCTATTTGTGGAAAATCCACTGGCGGTGAAGGTGAAG |
| | GTGGAGAAGGAGAGGGCGGAAGCCGGCAGTTGTACTCTGCCCTGGCTAATAAGTGC |
| | TGTCACGTGGGCTGCACTAAGCGGAGCTTGGCAAGATTTTGC |
| 493 | GATAAAACTCATACCTGTCCACCTTGTCCTGCGCCTGAGGCAGCTGGAGGGCCTAG |
| | CGTGTTCCTGTTCCCCCCCAAACCCAAAGACACGCTCATGATTAGCCGAACCCCTG |
| | AAGTGACCTGCGTTGTTGTGGACGTAAGCCACGAAGACCCCGAAGTTAAGTTTAAT |
| | TGGTACGTCGACGGTGTTGAGGTTCATAACGCGAAGACTAAGCCGAGAGAGGAGCA |
| | ATATAACAGCACCTACCGCGTAGTCTCAGTTCTTACCGTGCTCCACCAGGACTGGC |
| | TTAACGGGAAGGAATACAAATGCAAAGTTTCCAACAAAGCCTTGGCAGCCCCAATA |
| | GAGAAGACAATATCTAAGGCGAAAGGCCAACCGCGGGAACCGCAAGTTTATACCCT |
| | CCCACCGAGCAGGGATGAGCTGACAAAAAATCAGGTTTCCCTCACTTGTCTGGTCA |
| | AGGGATTTTATCCTTCAGACATAGCCGTTGAATGGGAGAGTAATGGGCAGCCGGAG |
| | AATAATTACAAGACCACCCCCCCGGTGTTGGACAGCGACGGTTCCTTCTTTCTCTA |
| | TTCTAAACTTACCGTCGACAAATCACGGTGGCAACAAGGAAATGTATTCTCATGCA |
| | GTGTATTGCACGAAGCTCTGCACTCTCATTACACCCAAAAATCCCTCTCTCTCAGC |
| | CCTGGCGGTGGATCTGATTCTTGGCAGGAAGAGGTGATTAAACTGTGTGGGCGAGA |
| | GCTTGTCCGAGCTCAGATCGCTATTTGTGGCAAGAGTACCGGAGGCGAGGGTGAGG |
| | GAGGCGAAGGCGAGGGCGGAAGCCGGCAACTCTATAGCGCACTCGCTAATAAATGT |
| | TGTCATGTCGGCTGCACGAAGCGCTCACTGGCGCAGTTCTGC |
| 499 | GATAAAACCCATACGTGTCCTCCATGCCCAGCTCCCGAGCTGCTCGGTGGTCCTTC |
| | AGTGTTCCTCTTCCCCCCAAAGCCGAAGGACACGCTCATGATTAGTCGAACGCCAG |
| | AGGTGACATGTGTGGTCGTTGATGTTTCCCATGAGGATCCGGAAGTTAAGTTCAAC |
| | TGGTACGTAGATGGCGTGGAGGTTCACAATGCAAAAACCAAGCCCCGCGAGGAGCA |
| | GTATAACTCAACCTACAGAGTAGTATCTGTGCTCACGGTCTTGCATCAGGATTGGT |
| | TGAACGGGAAGGAATACAAGTGTAAAGTAAGTAATAAGGCACTGCCGGCCCCCATA |
| | GAAAAAACTATCAGCAAAGCTAAAGGTCAGCCGCGGGAGCCACAAGTTTACACTCT |
| | TCCTCCTAGTAGAGACGAGCTGACGAAGAATCAAGTTTCTTTGACTTGTCTCGTGA |
| | AGGGATTCTACCCAAGCGATATAGCTGTAGAGTGGGAAAGCAACGGACAACCAGAA |
| | AATAACTACAAGACTACACCCCCCGTTCTCGATTCTGATGGCTCATTCTTCTTGTA |
| | CTCAAAATTGACAGTTGACAAATCTCGATGGCAGCAGGGTAACGTATTTAGTTGCT |
| | CTGTTATGCACGAAGCGTTGCATAACCACTACACACAGAAGTCATTGTCACTGAGC |
| | CCAGGAAAAGGTGGTGGCGGGTCCGGCGGTGGAGGTAGCGGTGGCGGGGGCTCCCA |
| | GCTTTATAGTGCCCTTGCAAACAAATGTTGCCACGTCGGATGTACGAAGCGCAGTT |
| | TGGCGAGATTCTGTGGAGGGGGCGGATCCGGAGGCGGGGGGTCCGGAGGAGGAGGT |
| | AGCTCATGGATGGAAGAGGTAATAAAACTGTGCGGACGCGAGCTTGTCAGGGCCCA |
| | AATCGCAATTTGTGGCATGAGCACATGGAGT |
| 532 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGAGAAGGGGGCGGAAGGCAGCTTTACTCTGCTCTGGCTAACAAG |
| | TGTTGTCACGTTGGGTGTACCAAGCGGTCCCTTGCGCAATTCTGC |
| 533 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGAGAAGGGGGCGGAAGGCAGCTTTACTCTGCTCTGGCTAACAAG |
| | TGTTGTCAAGTTGGGTGTACCAAGCGGTCCCTTGCGCAATTCTGC |
| 534 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGAGAAGGGGGCGGAAGGCAGCTTTACTCTGCTCTGGCTAACAAG |
| | TGTTGTCACGTTGGGTGTACCAAGCAATCCCTTGCGCAATTCTGC |
| 535 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGAGAAGGGGGCGGAAGGCAGCTTTACTCTGCTCTGGCTAACAAG |
| | TGTTGTCAAGTTGGGTGTACCAAGCAATCCCTTGCGCAATTCTGC |
| 536 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGATCTGGGGGCGGAAGGCAGCTTTACTCTGCTCTGGCTAACAAG |
| | TGTTGTCACGTTGGGTGTACCAAGCGGTCCCTTGCGCAATTCTGC |
| 537 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGATCTGGGGGCGGAAGGCAGCTTTACTCTGCTCTGGCTAACAAG |
| | TGTTGTCAAGTTGGGTGTACCAAGCGGTCCCTTGCGCAATTCTGC |
| 538 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGATCTGGGGGCGGAAGGCAGCTTTACTCTGCTCTGGCTAACAAG |
| | TGTTGTCACGTTGGGTGTACCAAGCAATCCCTTGCGCAATTCTGC |
| 539 | GATAAAACCCACACTTGCCCACCTTGCCCTGCGCCGGAAGCCGCCGGAGGACCTAG |
| | TGTTTTCCTCTTTCCCCCTAAGCCCAAAGACACGTTGATGATCTCTCGGACACCGG |
| | AAGTAACTTGTGTCGTTGTGGATGTGTCACATGAGGATCCCGAGGTGAAATTTAAT |
| | TGGTACGTTGACGGCGTGGAGGTGCATAACGCAAAGACTAAACCACGCGAGGAGCA |
| | GTATAATTCTACATACCGGGTTGTCTCAGTTCTCACAGTTCTTCATCAGGATTGGT |
| | TGAATGGAAAGGAGTACAAATGCAAAGTGTCCAACAAAGCGCTTGCTGCGCCGATT |
| | GAAAAGACGATTTCAAAGGCAAAAGGGCAGCCCCGCGAACCCCAAGTATATACTTT |
| | GCCTCCCTCACGCGATGAACTGACTAAGAACCAGGTGAGCCTGACTTGTTTGGTTA |
| | AGGGTTTTTATCCAAGTGACATTGCTGTTGAATGGGAGTCCAATGGCCAGCCTGAG |
| | AATAACTACAAAACGACACCTCCTGTACTTGACAGCGACGGCTCCTTTTTTCTTTA |
| | TTCAAAACTCACAGTGGACAAATCCAGGTGGCAGCAGGGTAACGTCTTTTCTTGCA |
| | GCGTGCTCCACGAAGCTTTGCATTCACATTATACGCAAAAATCCTTGTCATTGTCC |
| | CCAGGTAAGGGCGGAAGCGACTCATACCAAGAAGAAGTCATTAAACTTTGTGGACG |
| | GGAGCTGGTTAGGGCACAGATTGCTATTTGTGGTAAGTCTACGGGAGGAGAAGGCT |
| | CTGGAGGCGAAGGATCTGGGGGCGGAAGGCAGCTTTACTCTGCTCTGGCTAACAAG |
| | TGTTGTCAAGTTGGGTGTACCAAGCAATCCCTTGCGCAATTCTGC |

In some embodiments, the vector comprises a polynucleotide sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or 99% identical to a sequence recited in Table 10, below. In some embodiments, the vector comprises a polynucleotide sequence that comprises or consists of a sequence recited in Table 10, below.

TABLE 10
Nucleotide Sequences Encoding Fusion Proteins and Peptide Components

| SEQ | |
|---|---|
| ID | |
| NO: | Sequence |
| 194 | ATGGAGACTGACACACTCCTCCTTTGGGTTTTGCTGTTGTGGGTGCCTGGATCTAC |
| | TGGCGACAAGACCCATACATGTCCGCCTTGTCCTGCGCCTGAGGCAGCAGGCGGAC |
| | CATCAGTCTTCTTGTTTCCCCCCAAGCCGAAGGACACCCTTATGATCTCACGCACC |
| | CCCGAAGTAACTTGTGTAGTCGTTGATGTCTCACACGAAGACCCGGAAGTAAAGTT |
| | TAATTGGTATGTCGATGGTGTTGAGGTCCACAACGCTAAAACGAAACCGCGGGAAG |
| | AACAATACAACTCCACATATCGAGTAGTCTCCGTCCTGACTGTTCTTCACCAGGAC |
| | TGGCTGAATGGTAAAGAATACAAATGTAAAGTGAGTAACAAGGCCCTTGCAGCACC |
| | CATCGAGAAGACGATATCCAAAGCCAAGGGGCAACCGCGCGAGCCACAAGTTTACA |
| | CGCTCCCACCCTCAAGAGACGAACTCACCAAAAATCAAGTGTCCCTGACATGTCTG |
| | GTGAAAGGATTCTATCCCAGCGACATAGCTGTAGAATGGGAGAGTAATGGCCAACC |
| | CGAAAACAATTACAAAACTACCCCCCCGGTTTTGGATAGTGATGGTTCATTCTTCC |
| | TCTATAGTAAACTTACCGTGGATAAGTCTCGGTGGCAGCAGGGGAACGTGTTTAGC |
| | TGTTCAGTCCTCCATGAGGCACTCCATAGTCACTATACGCAAAAGTCATTGTCCCT |
| | TTCTCCGGGCAAGGGCGGGTCAGACTCCTGGCAGGAAGAGGTAATTAAGCTTTGTG |
| | GGCGAGAACTCGTTAGGGCACAGATAGCAATCTGCGGGAAAAGTACAGCTTCCGAT |
| | GCTGCCGGGGCTGACGCCAATGCGGGAGCACGCCAGCTCTACTCAGCCCTCGCCAA |
| | CAAGTGTTGTCATGTAGGTTGCACCAAAAGAAGTCTGGCACAGTTTTGC |
| 195 | ATGGAAACAGACACTCTTCTTTTGTGGGTCTTGTTGCTCTGGGTCCCTGGCTCAAC |
| | AGGCGACAAGACGCATACTTGTCCTCCCTGCCCAGCTCCCGAAGCGGCTGGGGGGC |
| | CCTCTGTCTTTCTGTTTCCGCCTAAGCCCAAGGACACGCTCATGATAAGTCGCACT |
| | CCGGAAGTCACCTGTGTTGTCGTCGATGTTAGCCATGAAGATCCAGAGGTGAAATT |
| | TAACTGGTACGTCGACGGAGTGGAGGTTCACAATGCTAAAACCAAACCGCGAGAAG |
| | AGCAATACAATTCCACGTATAGGGTCGTCTCCGTCCTGACAGTACTCCATCAGGAT |
| | TGGCTGAATGGAAAAGAATACAAGTGCAAGGTTTCCAATAAAGCCTTGGCCGCACC |
| | TATTGAGAAAACGATATCAAAAGCTAAGGGACAACCTCGGGAGCCGCAAGTATATA |
| | CACTCCCCCCTTCTAGGGACGAACTGACAAAAAACCAAGTTAGTCTGACTTGTCTT |
| | GTGAAAGGTTTTTACCCGAGTGATATAGCCGTAGAATGGGAGAGCAATGGCCAGCC |
| | CGAAAACAATTACAAAACAACTCCCCCAGTATTGGACAGTGACGGGTCATTTTTTT |
| | TGTATTCTAAATTGACCGTAGACAAGTCACGCTGGCAACAAGGGAATGTATTTAGC |
| | TGTTCCGTCCTTCATGAGGCGCTCCATAGCCATTACACTCAGAAGTCTTTGTCACT |
| | GTCACCGGGCAAGGGTGGTTCTGATTCATGGCAAGAGGAAGTGATTAAGCTGTGCG |
| | GTCGGGAGTTGGTAAGAGCTCAAATTGCGATTTGTGGCAAGAGCACTGCGTCCGAT |
| | GCCGCAGGTGCTAATGCCGACGCCGGTGCGAGACAGCTTTATTCTGCGCTGGCCAA |
| | CAAGTGCTGCCACGTCGGATGCACCAAACGGAGCCTTGCTCAGTTTTGC |
| 196 | ATGGAAACCGACACCCTGCTTCTCTGGGTGCTGCTGCTCTGGGTACCAGGGTCTAC |
| | AGGTGACAAAACTCATACTTGTCCACCATGCCCAGCCCCCGAGGCGGCTGGCGGCC |
| | CCAGCGTATTCCTTTTCCCCCCAAAACCTAAGGACACGCTTATGATATCTAGAACC |
| | CCGGAGGTCACATGTGTCGTCGTAGACGTAAGTCACGAAGATCCTGAAGTCAAGTT |
| | TAACTGGTACGTCGATGGAGTCGAGGTCCATAATGCTAAAACGAAGCCTCGCGAAG |
| | AACAGTATAATTCTACCTATCGCGTAGTCTCTGTCCTCACCGTCTTGCATCAAGAC |
| | TGGTTGAACGGCAAGGAGTACAAGTGTAAGGTTTCAAACAAAGCCCTTGCCGCGCC |
| | GATAGAGAAAACAATTAGCAAAGCGAAGGGGCAGCCGAGAGAGCCGCAAGTGTATA |
| | CCCTTCCTCCTAGTAGAGACGAGTTGACCAAAAACCAGGTGTCACTTACATGCCTC |
| | GTGAAAGGCTTCTACCCGAGTGATATTGCAGTCGAGTGGGAATCCAACGGCCAGCC |
| | CGAGAATAACTACAAAACGACGCCGCCCGTACTGGACAGTGATGGAAGTTTTTTTT |
| | TGTACTCAAAACTCACGGTTGACAAAAGTCGGTGGCAGCAAGGGAACGTTTTTAGC |
| | TGCTCTGTCCTCCATGAAGCACTCCATTCTCATTATACCCAGAAGTCTCTGTCTCT |
| | CTCCCCTGGTAAGGGAGGTTCTGACAGTTGGCAGGAAGAGGTAATAAAACTCTGCG |
| | GTCGAGAGCTTGTTCGAGCACAAATTGCTATATGTGGAAAATCTACCGCTTCAGAC |
| | GCCGCCGGAGCTGATGCGGATGCCGGGGCTCGCCAGCTCTATAGCGCCTTGGCCAA |
| | CAAATGTTGTCACGTTGGCTGCACGAAGCGCTCCCTGGCTCAGTTTTGC |
| 197 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATGGCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTCACGTTGGGTGTACCAAGCGGTCCCTTGCGCAATTCTGC |
| 198 | ATGGAGACTGATACACTTCTTCTCTGGGTCCTCCTCCTCTGGGTTCCAGGCTCAAC |
| | AGGTGATAAAACTCATACTTGCCCCCCCTGCCCCGCGCCTGAAGCTGCAGGGGGGC |
| | CATCAGTCTTCTTGTTTCCACCAAAACCTAAGGATACTCTCATGATTAGCCGGACC |
| | CCTGAGGTGACATGTGTTGTGGTCGATGTATCTCATGAAGATCCCGAAGTAAAATT |
| | TAACTGGTACGTAGACGGGGTTGAAGTTCATAACGCGAAAACGAAACCTCGGGAGG |
| | AGCAATATAATAGCACGTATAGAGTTGTTTCAGTCCTTACAGTTCTCCACCAAGAC |
| | TGGCTGAATGGCAAGGAGTATAAGTGTAAAGTATCCAATAAAGCCTTGGCTGCGCC |
| | AATCGAGAAGACGATCAGCAAAGCCAAAGGTCAGCCTCGCGAACCGCAGGTCTATA |
| | CATTGCCCCCTTCACGCGACGAACTCACGAAAAATCAAGTCTCTTTGACTTGCCTT |
| | GTGAAAGGCTTCTACCCCTCCGATATTGCCGTCGAATGGGAAAGCAATGGACAGCC |
| | GGAAAATAATTACAAAACGACACCCCCCGTGTTGGATTCCGATGGGTCCTTCTTCC |
| | TCTATTCCAAGCTGACGGTCGATAAGTCTCGATGGCAGCAGGGAAATGTCTTCTCT |
| | TGCTCCGTCCTTCATGAGGCATTGCACAGCCATTATACTCAAAAGAGTCTCTCTCT |
| | GTCTCCAGGCAAAGGGGGTTCCGACTCTTGGCAAGAAGAGGTCATAAAACTGTGCG |
| | GCCGGGAGCTCGTCAGAGCGCAGATCGCTATATGTGGAAAATCCACCGCGAGTGAC |
| | GCAGCAGGTGCACAAGCCGACGCAGGAGCTAGGCAACTGTACTCAGCCCTTGCCAA |
| | TAAGTGTTGTCACGTAGGTTGTACTAAACGCTCCCTGGCACAATTTTGT |
| 199 | ATGGAAACCGATACCTTGCTCCTTTGGGTTCTGCTCCTTTGGGTCCCTGGAAGTAC |
| | AGGGGATAAGACACACACGTGCCCACCCTGCCCTGCCCCAGAGGCAGCCGGAGGTC |
| | CTAGTGTGTTTCTGTTCCCCCCAAAGCCCAAGGACACCCTTATGATATCTAGGACA |
| | CCAGAAGTTACGTGCGTCGTTGTGGACGTTAGCCACGAAGACCCAGAAGTGAAGTT |
| | TAATTGGTACGTTGATGGAGTCGAAGTGCACAATGCAAAAACAAAACCACGAGAAG |
| | AGCAGTATAACAGTACTTATAGAGTAGTCAGCGTCCTTACTGTATTGCATCAGGAT |
| | TGGCTGAATGGGAAGGAATATAAATGTAAGGTTAGCAATAAAGCCCTTGCGGCTCC |
| | TATCGAGAAAACTATTAGCAAAGCAAAGGGCCAACCCCGAGAGCCCCAAGTTTATA |
| | CACTGCCACCCAGTCGAGATGAGCTGACTAAAAATCAAGTATCCCTGACCTGCTTG |
| | GTTAAGGGGTTTTATCCTAGTGACATCGCGGTTGAGTGGGAATCCAACGGCCAACC |
| | GGAGAATAATTACAAAACCACGCCACCTGTATTGGATTCCGATGGTAGCTTCTTTC |
| | TCTATAGTAAACTTACAGTCGATAAGTCAAGATGGCAGCAGGGAAACGTATTTTCA |
| | TGCTCAGTTCTGCATGAGGCCTTGCACTCCCATTACACTCAAAAATCACTGAGCCT |
| | CAGTCCTGGTAAGGGTGGCTCTGACTCATGGCAGGAGGAAGTAATCAAGCTGTGTG |
| | GGAGGGAATTGGTAAGGGCTCAGATTGCAATTTGTGGAAAGAGCACAGCGTCTGAC |
| | GCTGCAGGTGCCGACGCACAGGCGGGCGCGAGGCAGCTCTACAGTGCTCTTGCGAA |
| | CAAGTGTTGTCATGTAGGTTGCACGAAACGAAGTTTGGCGCAATTCTGT |
| 200 | ATGGAGACTGACACTCTCCTTCTTTGGGTGCTGTTGCTCTGGGTTCCCGGAAGCAC |
| | GGGGGATAAAACACACACCTGCCCCCCTTGCCCAGCACCTGAAGCAGCGGGTGGTC |
| | CCAGCGTTTTTCTTTTCCCCCCTAAGCCAAAGGACACGCTCATGATAAGTCGCACC |
| | CCGGAGGTCACCTGCGTCGTTGTTGACGTATCACATGAAGATCCTGAGGTGAAGTT |
| | TAATTGGTACGTAGATGGTGTTGAGGTCCACAACGCAAAGACGAAACCGAGGGAAG |
| | AACAGTACAACAGTACTTATCGCGTAGTCTCCGTTCTGACTGTCCTGCATCAAGAT |
| | TGGTTGAACGGGAAGGAGTACAAGTGCAAAGTTAGTAACAAGGCTCTCGCGGCCCC |
| | AATTGAGAAGACGATATCCAAAGCGAAAGGACAGCCGAGAGAGCCCCAAGTCTACA |
| | CTCTGCCCCCTTCCAGGGATGAGCTCACCAAAAATCAGGTCAGTCTCACGTGCCTG |
| | GTTAAGGGATTCTACCCAAGTGATATAGCAGTTGAATGGGAGAGTAACGGCCAGCC |
| | CGAGAACAACTATAAAACTACACCGCCCGTTCTTGATTCCGATGGGTCTTTCTTCC |
| | TTTATAGTAAGCTCACCGTTGATAAGTCCCGATGGCAGCAAGGTAATGTCTTCTCA |
| | TGTTCAGTTCTTCATGAAGCCCTGCATTCCCATTATACACAAAAGAGCTTGTCCTT |
| | GTCACCGGGCAAAGGCGGTAGCGATTCTTGGCAAGAAGAAGTTATAAAGTTGTGCG |
| | GTAGGGAACTGGTACGCGCTCAAATAGCTATATGCGGTAAGTCTACTGCTTCAGAT |
| | GCGGCTGGCGCACAGGCACAGGCCGGTGCTAGACAACTCTATAGTGCGCTGGCCAA |
| | CAAGTGCTGCCATGTGGGGTGTACAAAACGGAGTCTTGCCCAGTTTTGT |
| 201 | ATGGAAACTGACACGCTTTTGCTTTGGGTCCTCCTTCTTTGGGTTCCCGGCTCTAC |
| | TGGAGACAAAACTCATACATGCCCCCCATGCCCAGCACCCGAAGCGGCCGGAGGTC |
| | CGTCTGTCTTTCTGTTTCCGCCGAAACCTAAAGATACGTTGATGATTAGCAGAACC |
| | CCTGAGGTAACATGTGTGGTAGTCGATGTCTCCCATGAGGACCCCGAGGTAAAGTT |
| | CAATTGGTATGTTGACGGCGTCGAAGTCCATAACGCAAAAACGAAGCCCCGAGAGG |
| | AGCAATATAACTCTACCTATCGCGTTGTTTCTGTTTTGACTGTGTTGCACCAGGAT |
| | TGGCTCAACGGCAAGGAATACAAATGTAAAGTGTCCAACAAGGCCCTTGCTGCACC |
| | TATCGAAAAAACGATTAGTAAGGCAAAGGGACAACCGCGCGAACCACAGGTATATA |
| | CTTTGCCGCCTAGCAGAGATGAACTCACCAAGAATCAAGTTTCCCTTACCTGTTTG |
| | GTTAAAGGATTTTACCCGTCTGACATAGCTGTTGAATGGGAGAGCAATGGTCAGCC |
| | GGAAAATAATTATAAAACCACCCCGCCAGTATTGGATTCAGATGGGTCCTTTTTCT |
| | TGTATTCTAAACTTACCGTGGATAAGTCTAGGTGGCAACAGGGAAACGTCTTTTCA |
| | TGTAGTGTACTTCATGAAGCCCTCCATAGTCACTACACGCAGAAATCCTTGTCTCT |
| | TAGTCCGGGTGAAGGTGGGTCTGATTCCTGGCAGGAAGAGGTGATAAAGCTCTGTG |
| | GTCGGGAACTTGTTAGGGCGCAGATCGCTATTTGCGGCAAATCTACAGCATCAGAT |
| | GCCGCCGGAGCTGATGCGAACGCAGGAGCGAGGCAGCTGTACTCCGCACTTGCTAA |
| | CAAGTGTTGCCATGTCGGCTGCACCAAGAGGAGTCTTGCTCAATTCTGC |
| 202 | ATGGAAACCGACACCCTCCTTCTTTGGGTCTTGCTGCTTTGGGTTCCTGGTTCTAC |
| | TGGAGATAAGACCCATACATGCCCGCCATGTCCCGCACCCGAGGCAGCGGGTGGAC |
| | CCTCTGTCTTTCTGTTCCCTCCAAAGCCAAAAGATACCCTGATGATTAGCCGAACC |
| | CCGGAGGTGACTTGTGTCGTAGTAGATGTCAGTCACGAGGATCCCGAAGTAAAGTT |
| | TAATTGGTATGTGGACGGTGTGGAGGTACATAACGCTAAGACGAAACCCCGAGAGG |
| | AACAATACAACTCTACGTACAGGGTCGTCTCAGTGCTCACGGTCCTGCACCAGGAC |
| | TGGCTTAATGGGAAGGAATATAAATGCAAAGTCTCTAATAAGGCGCTTGCTGCACC |
| | TATTGAAAAAACGATTTCTAAGGCGAAGGGACAACCCCGGGAGCCACAAGTCTACA |
| | CCCTTCCTCCAAGCAGAGATGAGCTTACGAAAAATCAAGTGTCTCTTACGTGCCTC |
| | GTAAAGGGCTTTTACCCATCCGACATTGCGGTGGAGTGGGAATCAAACGGGCAGCC |
| | GGAAAATAACTACAAAACAACGCCGCCTGTATTGGATTCCGACGGCTCTTTCTTCC |
| | TTTACAGCAAACTGACAGTCGATAAATCCAGATGGCAACAAGGGAACGTTTTTTCA |
| | TGTTCCGTTCTGCATGAAGCCCTTCACAGTCATTACACCCAAAAGTCACTTTCACT |
| | TTCACCGGGCGAGGGGGGGTCAGACTCCTGGCAAGAGGAAGTTATAAAGTTGTGCG |
| | GCAGGGAACTGGTTAGAGCGCAGATAGCGATTTGCGGAAAATCTACTGCGAGTGAT |
| | GCTGCGGGAGCGAATGCGGACGCCGGGGCCCGACAGCTCTATTCCGCACTCGCCAA |
| | TAAGTGCTGCCATGTTGGTTGTACGAAGAGAAGTCTTGCACAATTTTGC |
| 203 | ATGGAAACAGACACGCTGCTTTTGTGGGTACTGTTGCTTTGGGTCCCAGGATCCAC |
| | AGGTGATAAGACACATACATGCCCCCCCTGCCCGGCTCCCGAAGCTGCCGGGGGAC |
| | CGTCAGTGTTTTTGTTTCCGCCCAAGCCGAAGGATACTTTGATGATTAGTCGGACA |
| | CCAGAAGTGACATGTGTTGTCGTTGACGTGAGTCACGAGGATCCCGAGGTCAAGTT |
| | CAACTGGTACGTTGATGGGGTTGAAGTTCACAACGCTAAAACGAAACCCCGCGAAG |
| | AGCAGTATAACTCCACTTACCGGGTCGTCAGTGTCCTGACGGTCTTGCACCAGGAC |
| | TGGCTGAATGGAAAGGAATACAAGTGTAAAGTTTCCAATAAAGCACTGGCCGCCCC |
| | GATCGAAAAAACAATTTCCAAAGCTAAGGGACAGCCCAGGGAACCGCAAGTTTATA |
| | CTCTTCCACCCTCCCGGGATGAACTGACCAAAAACCAAGTGTCTTTGACGTGCCTC |
| | GTAAAGGGCTTCTACCCGTCAGACATAGCTGTCGAATGGGAGTCTAATGGACAGCC |
| | GGAAAACAATTATAAGACTACACCGCCGGTGCTTGATAGTGATGGAAGTTTCTTTT |
| | TGTACTCCAAACTTACGGTCGATAAAAGCCGGTGGCAGCAGGGAAACGTATTCAGT |
| | TGTAGCGTTCTGCATGAAGCTCTTCATTCTCACTACACCCAGAAGTCTCTGTCTCT |
| | GAGCCCCGGAGAGGGTGGATCTGATTCTTGGCAGGAAGAAGTGATAAAGTTGTGCG |
| | GCCGGGAATTGGTACGCGCCCAGATAGCCATTTGCGGGAAGTCTACGGCGAGTGAC |
| | GCAGCAGGTGCTGACGCGGACGCTGGTGCTAGACAGCTGTATTCTGCCCTGGCTAA |
| | TAAGTGTTGCCACGTTGGCTGCACCAAGAGATCCCTGGCCCAATTCTGT |
| 204 | ATGGAAACTGACACTTTGCTGTTGTGGGTCCTCCTGTTGTGGGTCCCCGGAAGTAC |
| | AGGAGACAAGACACATACTTGTCCCCCCTGCCCAGCTCCAGAAGCTGCCGGAGGGC |
| | CGTCAGTCTTCCTTTTCCCTCCAAAACCTAAGGATACGCTTATGATTTCTCGAACG |
| | CCAGAGGTTACGTGTGTAGTCGTGGACGTTTCCCACGAGGATCCTGAGGTCAAGTT |
| | TAACTGGTATGTAGACGGGGTTGAGGTCCATAATGCCAAGACAAAGCCGCGCGAGG |
| | AACAATACAACAGTACATATAGGGTGGTGAGCGTCCTCACAGTCTTGCATCAAGAT |
| | TGGCTCAACGGCAAAGAGTACAAATGTAAGGTTAGCAACAAAGCCCTCGCTGCTCC |
| | CATCGAAAAGACGATTTCTAAGGCGAAGGGCCAACCACGAGAACCGCAAGTATATA |
| | CTCTTCCCCCTTCACGGGACGAGCTGACCAAAAACCAGGTATCCTTGACTTGCCTG |
| | GTCAAAGGATTTTACCCCTCTGATATTGCGGTCGAGTGGGAGAGTAATGGGCAACC |
| | AGAAAATAATTATAAAACGACCCCCCCGGTACTCGACAGTGATGGGTCTTTTTTCC |
| | TGTATTCTAAGCTTACGGTTGATAAGTCTAGATGGCAGCAAGGGAATGTCTTCTCA |
| | TGTAGTGTTCTGCATGAAGCACTTCATTCTCACTATACTCAGAAATCTCTTTCCCT |
| | TAGTCCGGGAGAAGGTGGGAGCGATAGTTGGCAAGAGGAGGTGATAAAACTGTGTG |
| | GTCGGGAGCTGGTGAGAGCCCAAATAGCTATCTGCGGCAAATCAACAGCAAGTGAT |
| | GCGGCAGGAGCGGAAGCGGAGGCGGGAGCGCGGCAATTGTATAGTGCCCTTGCTAA |
| | TAAATGCTGTCACGTTGGGTGTACTAAACGATCTCTTGCTCAATTCTGC |
| 205 | ATGGAGACGGATACGTTGTTGCTTTGGGTCCTCCTGTTGTGGGTGCCCGGCTCTAC |
| | CGGTGATAAAACCCATACATGTCCTCCGTGTCCCGCTCCAGAAGCCGCTGGCGGGC |
| | CATCTGTGTTTTTGTTCCCCCCCAAGCCTAAGGATACGTTGATGATCAGCAGGACC |
| | CCGGAGGTTACATGCGTAGTAGTTGACGTTTCTCATGAAGACCCAGAAGTAAAATT |
| | TAACTGGTATGTCGATGGCGTCGAAGTACATAATGCTAAAACTAAGCCCAGGGAAG |
| | AGCAATACAATTCAACGTACCGAGTTGTGAGTGTCCTTACGGTCCTGCACCAAGAC |
| | TGGTTGAACGGCAAAGAGTACAAATGCAAAGTGTCTAACAAGGCATTGGCCGCGCC |
| | TATAGAAAAGACCATTAGCAAAGCAAAAGGGCAGCCTCGGGAACCCCAGGTCTACA |
| | CGCTGCCACCTTCCCGAGATGAATTGACGAAAAACCAGGTCTCTTTGACCTGCTTG |
| | GTTAAAGGCTTCTACCCAAGCGACATTGCAGTGGAGTGGGAGTCTAACGGGCAACC |
| | CGAAAACAACTATAAGACGACTCCCCCTGTTCTTGATTCTGATGGGAGTTTTTTTC |
| | TGTACAGTAAGTTGACAGTGGATAAATCAAGATGGCAGCAAGGTAATGTCTTCTCT |
| | TGTTCAGTGCTTCACGAAGCATTGCATTCTCACTACACACAAAAGTCTTTGTCCTT |
| | GTCTCCAGGTGAAGGCGGTAGCGATTCATGGCAAGAAGAAGTCATTAAGCTGTGTG |
| | GAAGGGAACTGGTTAGGGCCCAAATTGCGATATGTGGAAAGTCTACGGCGAGTGAT |
| | GCGGCCGGTGCTCAAGCGGATGCGGGTGCTAGACAGTTGTACTCAGCCCTTGCGAA |
| | CAAATGTTGTCACGTTGGCTGTACGAAACGCAGCCTTGCTCAATTCTGC |
| 206 | ATGGAAACTGACACCCTTCTGCTCTGGGTACTCTTGTTGTGGGTCCCCGGCAGTAC |
| | AGGCGATAAAACTCACACATGCCCCCCATGCCCAGCACCGGAAGCTGCCGGAGGAC |
| | CGTCTGTATTCCTCTTTCCGCCCAAACCGAAAGACACGTTGATGATTTCTCGGACT |
| | CCCGAGGTAACTTGTGTCGTGGTCGACGTCTCACACGAGGACCCGGAGGTCAAATT |
| | TAACTGGTATGTCGATGGGGTGGAGGTCCATAATGCTAAGACGAAGCCCAGAGAAG |
| | AACAGTATAACTCTACTTATAGAGTTGTAAGCGTGCTCACTGTATTGCACCAGGAC |
| | TGGCTCAACGGGAAAGAATATAAGTGTAAGGTCTCAAACAAAGCTCTCGCAGCCCC |
| | GATAGAGAAAACAATATCTAAGGCCAAGGGCCAACCGCGCGAGCCGCAGGTTTATA |
| | CACTTCCACCCTCCCGCGATGAGCTGACCAAGAACCAGGTCTCTCTCACCTGTCTC |
| | GTAAAGGGCTTTTATCCCTCCGACATTGCAGTGGAGTGGGAATCAAACGGCCAGCC |
| | GGAAAATAATTACAAGACCACTCCTCCCGTCCTCGACTCCGATGGGTCATTTTTCC |
| | TGTACAGTAAGCTCACCGTTGATAAGTCAAGGTGGCAGCAGGGCAACGTGTTTAGC |
| | TGTAGTGTTCTGCATGAGGCGCTCCACAGTCACTACACCCAGAAAAGTCTGAGCCT |
| | TTCCCCAGGTGAGGGTGGTAGCGATAGCTGGCAGGAGGAAGTAATTAAACTCTGCG |
| | GTAGAGAATTGGTAAGGGCCCAAATTGCCATCTGCGGAAAGAGCACCGCATCAGAT |
| | GCTGCGGGCGCGGATGCGCAGGCTGGTGCTAGGCAACTCTACTCTGCCCTGGCGAA |
| | TAAATGTTGCCACGTCGGTTGCACGAAACGAAGTTTGGCTCAATTTTGC |
| 207 | ATGGAAACGGACACACTTCTCTTGTGGGTTCTTCTGCTCTGGGTTCCCGGCAGTAC |
| | AGGCGACAAAACACATACATGCCCCCCTTGCCCGGCTCCCGAGGCCGCCGGTGGTC |
| | CTAGCGTCTTTCTTTTCCCTCCCAAACCCAAAGACACACTTATGATTAGCAGAACT |
| | CCCGAGGTAACATGTGTGGTCGTAGACGTAAGTCACGAAGATCCCGAAGTTAAATT |
| | CAACTGGTACGTTGATGGTGTGGAAGTTCATAATGCAAAAACCAAACCGCGAGAGG |
| | AACAGTATAACTCTACCTACCGCGTGGTCTCAGTGCTGACTGTCCTGCATCAGGAC |
| | TGGCTCAACGGGAAGGAATATAAGTGCAAAGTGAGTAATAAGGCCCTTGCAGCTCC |
| | CATAGAAAAGACGATATCAAAGGCTAAAGGACAGCCGAGGGAGCCACAGGTGTACA |
| | CTTTGCCTCCGAGTAGAGATGAACTCACTAAAAACCAAGTAAGTTTGACATGCCTG |
| | GTCAAAGGTTTTTACCCCAGTGATATAGCGGTTGAGTGGGAGTCCAATGGGCAACC |
| | GGAGAACAACTATAAGACTACTCCACCTGTCCTGGATAGCGATGGAAGTTTTTTTC |
| | TTTACTCAAAGCTGACGGTGGATAAGAGTCGATGGCAGCAGGGCAATGTGTTTAGC |
| | TGTTCTGTGCTTCACGAAGCACTTCACTCTCATTATACCCAGAAGTCATTGAGCCT |
| | TTCCCCTGGTGAAGGAGGGTCAGATTCCTGGCAGGAGGAGGTTATAAAGCTGTGTG |
| | GCCGGGAACTCGTGCGAGCTCAAATTGCGATCTGTGGAAAATCCACCGCTAGTGAT |
| | GCGGCGGGAGCACAAGCTCAAGCGGGCGCTCGACAACTTTATAGCGCTTTGGCTAA |
| | TAAGTGCTGCCATGTGGGTTGTACAAAGCGCAGCCTCGCTCAATTTTGC |
| 208 | ATGGAAACAGACACTCTCCTCCTGTGGGTACTTCTCTTGTGGGTACCAGGATCCAC |
| | CGGGGATAAGACGCACACTTGCCCTCCTTGCCCGGCACCCGAAGCCGCTGGTGGGC |
| | CTAGTGTATTCCTGTTCCCCCCGAAGCCGAAGGATACTCTTATGATTTCACGCACG |
| | CCCGAGGTTACATGCGTAGTAGTGGACGTATCTCACGAAGATCCCGAAGTCAAGTT |
| | CAATTGGTATGTCGACGGAGTAGAAGTTCACAACGCAAAGACAAAACCGCGGGAAG |
| | AGCAATACAACTCCACGTACCGCGTCGTTTCTGTTCTTACGGTCTTGCACCAGGAC |
| | TGGCTCAATGGCAAGGAGTATAAGTGCAAGGTATCCAACAAGGCCCTTGCCGCACC |
| | TATTGAAAAGACTATCAGCAAGGCCAAGGGACAGCCAAGGGAGCCTCAAGTCTACA |
| | CGCTCCCGCCTAGTAGAGACGAGTTGACAAAGAATCAAGTGAGTTTGACTTGTCTG |
| | GTTAAAGGTTTTTACCCGTCAGATATTGCAGTAGAATGGGAATCTAACGGACAACC |
| | CGAAAACAACTATAAAACGACGCCTCCTGTGTTGGATTCAGATGGGTCATTTTTTC |
| | TCTACTCAAAGCTCACGGTAGATAAATCAAGATGGCAACAAGGCAATGTATTTTCC |
| | TGCTCCGTGCTCCACGAGGCTCTGCACAGCCATTATACGCAAAAGAGTCTGTCTTT |
| | GAGCCCAGGTGAGGGTGGCTCCGATTCCTGGCAGGAGGAAGTAATTAAGTTGTGCG |
| | GCAGGGAACTTGTTCGCGCACAAATAGCCATTTGTGGTCAGAGCACAGCATCAGAT |
| | GCCGCCGGAGCCGACGCCAACGCAGGTGCCCGCCAACTTTATTCTGCCCTCGCAAA |
| | CAAATGCTGCCACGTCGGCTGCACGAAGAGGAGCCTCGCCCAATTTTGC |
| 209 | ATGGAAACAGACACGCTGCTCCTCTGGGTGTTGCTTCTCTGGGTGCCTGGCAGTAC |
| | AGGTGATAAGACCCATACGTGCCCGCCATGTCCAGCCCCCGAGGCAGCCGGAGGTC |
| | CTTCCGTTTTCCTTTTCCCCCCTAAGCCCAAGGACACTCTGATGATCTCCCGGACG |
| | CCTGAAGTCACTTGCGTAGTCGTAGACGTTTCACATGAGGATCCAGAAGTTAAATT |
| | TAACTGGTACGTCGATGGCGTCGAGGTCCATAACGCGAAAACCAAGCCCAGGGAGG |
| | AACAATATAACTCCACCTATAGGGTCGTGAGTGTGCTCACCGTTTTGCACCAAGAC |
| | TGGCTCAACGGGAAAGAGTACAAATGTAAAGTTTCAAATAAGGCTTTGGCCGCCCC |
| | AATAGAGAAGACTATATCCAAGGCTAAGGGACAGCCTCGAGAACCGCAGGTATATA |
| | CGCTTCCTCCGTCTAGGGATGAACTCACAAAAAACCAGGTTTCTTTGACCTGCTTG |
| | GTAAAGGGATTTTATCCCTCCGACATTGCGGTCGAATGGGAGAGCAACGGACAGCC |
| | GGAAAACAATTACAAAACGACACCCCCGGTTTTGGACTCTGATGGAAGCTTCTTCC |
| | TCTATAGTAAGTTGACCGTAGACAAGTCTCGCTGGCAGCAGGGAAACGTCTTCAGT |
| | TGCTCAGTTCTCCATGAGGCGTTGCATAGTCACTATACACAGAAGAGTCTTAGTTT |
| | GTCTCCAGGAGAAGGAGGTTCTGATTCTTGGCAAGAGGAGGTAATCAAATTGTGTG |
| | GCCGAGAACTTGTTAGAGCTCAGATAGCCATCTGCGGACAGTCTACGGCGTCCGAT |
| | GCGGCCGGAGCTAATGCTGACGCAGGTGCGCGACAGCTGTACTCCGCACTGGCGAA |
| | TAAGTGCTGCCACGTGGGATGCACTAAGCGGTCTCTCGCGCAATTCTGT |
| 210 | ATGGAGACCGACACTCTTTTGCTCTGGGTGCTGTTGCTGTGGGTTCCAGGTTCAAC |
| | GGGAGATAAAACCCACACCTGTCCACCATGCCCGGCGCCGGAAGCCGCCGGGGGAC |
| | CCAGCGTATTTCTTTTCCCCCCCAAGCCCAAAGACACGCTGATGATTTCACGAACG |
| | CCGGAGGTGACTTGCGTGGTAGTGGACGTCTCCCATGAGGATCCCGAAGTTAAATT |
| | TAATTGGTATGTAGATGGTGTTGAGGTCCATAATGCTAAAACAAAGCCGCGGGAAG |
| | AGCAATATAACTCCACCTATAGAGTGGTCTCTGTACTCACTGTCCTGCACCAGGAT |
| | TGGCTGAATGGGAAAGAGTACAAGTGTAAAGTTAGCAACAAAGCGCTCGCCGCGCC |
| | TATCGAAAAAACGATTTCCAAAGCAAAGGGCCAACCACGAGAACCCCAGGTTTACA |
| | CCCTGCCACCCAGTCGAGATGAACTCACTAAGAATCAGGTGTCCCTTACATGCCTC |
| | GTCAAGGGATTCTATCCGAGCGATATAGCGGTGGAATGGGAGAGTAACGGTCAACC |
| | CGAAAATAACTATAAAACCACTCCGCCGGTACTCGATTCTGACGGTTCCTTCTTTC |
| | TTTATTCCAAACTGACTGTAGACAAATCACGGTGGCAGCAGGGCAACGTGTTTAGC |
| | TGCTCTGTACTCCATGAGGCCTTGCATTCTCATTATACTCAAAAGAGTCTGAGTCT |
| | GAGTCCAGGTGAAGGGGGTTCCGATTCATGGCAAGAGGAAGTCATTAAACTCTGCG |
| | GAAGGGAACTTGTAAGAGCACAAATCGCGATTTGTGGGCAATCTACCGCATCCGAC |
| | GCGGCTGGAGCAGATGCAGATGCCGGAGCGAGGCAGCTGTATTCAGCATTGGCTAA |
| | CAAATGTTGCCATGTTGGATGTACGAAGAGATCACTTGCACAGTTCTGT |
| 211 | ATGGAAACTGACACCCTCCTTCTCTGGGTACTGCTCTTGTGGGTTCCAGGCTCTAC |
| | TGGCGATAAGACTCATACCTGCCCGCCCTGTCCCGCACCCGAGGCTGCCGGAGGGC |
| | CATCAGTGTTCCTTTTCCCACCAAAGCCGAAGGATACACTTATGATCAGCAGGACA |
| | CCCGAAGTGACCTGTGTAGTCGTAGACGTGTCCCACGAAGACCCCGAAGTAAAATT |
| | TAATTGGTATGTCGATGGCGTAGAGGTCCACAACGCGAAAACGAAACCCCGCGAAG |
| | AACAATATAATTCCACATACCGAGTTGTCAGCGTCCTCACTGTTCTCCATCAGGAC |
| | TGGCTGAATGGGAAGGAATATAAGTGCAAGGTCTCAAACAAGGCGCTGGCGGCCCC |
| | CATAGAGAAAACGATTTCTAAGGCCAAAGGACAGCCACGGGAACCGCAGGTCTATA |
| | CGCTCCCACCTAGTAGGGATGAGTTGACCAAGAATCAGGTATCCCTCACATGTCTC |
| | GTCAAGGGATTCTATCCCAGCGACATAGCCGTGGAGTGGGAATCTAACGGTCAACC |
| | TGAGAATAACTATAAAACAACCCCCCCGGTCCTCGACTCCGATGGTAGCTTCTTTC |
| | TGTATTCCAAACTGACGGTAGATAAAAGCCGATGGCAACAGGGTAACGTCTTTAGT |
| | TGTTCTGTATTGCACGAGGCGCTCCATAGTCACTACACACAGAAGTCTTTGAGCCT |
| | CTCACCTGGGGAGGGGGGTAGCGATTCTTGGCAGGAGGAAGTGATCAAACTGTGCG |
| | GCAGGGAACTGGTCAGAGCACAGATAGCAATATGCGGTCAGAGTACGGCCTCTGAC |
| | GCCGCCGGTGCGGAGGCTGAGGCAGGGGCGAGACAGCTCTACAGCGCTCTTGCAAA |
| | TAAGTGTTGTCACGTGGGGTGCACAAAGAGATCCTTGGCGCAATTTTGT |
| 212 | ATGGAAACCGATACATTGCTTTTGTGGGTCTTGTTGCTGTGGGTGCCCGGTTCTAC |
| | TGGTGATAAGACCCACACATGTCCGCCATGTCCAGCCCCAGAGGCAGCAGGGGGCC |
| | CGTCCGTATTCTTGTTTCCCCCGAAACCCAAAGATACCCTTATGATTAGTCGAACT |
| | CCAGAAGTCACGTGTGTGGTGGTGGACGTATCCCACGAGGACCCCGAAGTGAAATT |
| | CAATTGGTATGTGGACGGGGTGGAAGTCCATAACGCTAAGACGAAGCCCAGAGAGG |
| | AGCAGTACAATTCTACCTATCGGGTTGTATCTGTGCTTACTGTTCTCCATCAAGAT |
| | TGGCTGAACGGGAAGGAATACAAATGTAAAGTTAGTAACAAAGCATTGGCAGCTCC |
| | TATCGAAAAGACGATAAGCAAGGCTAAAGGTCAACCCCGAGAGCCTCAGGTCTACA |
| | CTTTGCCGCCCTCCAGGGATGAGCTTACCAAGAACCAAGTGAGCTTGACGTGTCTC |
| | GTGAAGGGATTCTACCCATCAGATATAGCGGTAGAATGGGAGTCTAATGGGCAGCC |
| | CGAGAACAACTATAAGACCACCCCTCCCGTTCTTGACTCCGACGGTTCCTTTTTCT |
| | TGTACTCCAAACTCACGGTCGACAAGTCTAGGTGGCAGCAAGGCAATGTTTTCAGT |
| | TGTTCCGTGCTGCACGAAGCTCTTCATTCTCACTATACGCAAAAAAGCCTGAGTCT |
| | TTCACCTGGAGAGGGGGGTTCCGATTCTTGGCAGGAAGAAGTCATTAAGCTGTGCG |
| | GCAGAGAACTTGTGCGCGCACAAATTGCTATTTGTGGACAGTCAACTGCATCTGAC |
| | GCCGCTGGAGCCCAAGCGGACGCAGGGGCAAGGCAGCTTTATTCAGCGCTTGCGAA |
| | TAAGTGTTGCCATGTGGGTTGCACGAAACGAAGCCTGGCGCAATTTTGT |
| 213 | ATGGAGACTGATACATTGTTGCTTTGGGTACTCTTGCTTTGGGTGCCCGGAAGTAC |
| | AGGGGATAAGACACATACATGTCCTCCCTGTCCCGCTCCGGAGGCAGCCGGTGGGC |
| | CTTCAGTTTTCTTGTTTCCGCCGAAGCCTAAGGACACGTTGATGATATCCCGAACA |
| | CCAGAGGTCACATGCGTCGTCGTGGACGTCTCACACGAGGACCCTGAAGTGAAATT |
| | CAACTGGTATGTAGACGGGGTCGAAGTTCACAATGCGAAAACTAAACCTCGCGAGG |
| | AGCAATATAACTCAACATACCGCGTAGTGTCCGTCTTGACTGTCCTTCATCAGGAT |
| | TGGCTGAATGGTAAAGAATATAAATGTAAAGTTTCTAATAAAGCGCTTGCGGCACC |
| | CATTGAGAAGACAATTTCCAAAGCCAAAGGCCAACCCCGAGAGCCTCAGGTATATA |
| | CGCTGCCTCCGTCTCGAGATGAGTTGACAAAAAATCAAGTCAGCTTGACTTGTCTT |
| | GTAAAGGGGTTCTATCCGTCAGACATAGCAGTGGAGTGGGAATCCAACGGGCAACC |
| | AGAAAATAATTACAAAACCACTCCGCCCGTGCTTGACTCAGATGGGAGCTTCTTCC |
| | TTTATAGCAAACTTACGGTAGATAAATCCAGATGGCAGCAAGGCAACGTATTCAGC |
| | TGTAGTGTGCTGCATGAAGCGCTTCACTCCCATTATACTCAAAAATCTCTTTCTCT |
| | GTCACCGGGCGAGGGCGGAAGTGATAGTTGGCAGGAAGAGGTCATCAAGCTCTGTG |
| | GGAGAGAGCTTGTACGCGCTCAGATTGCTATATGCGGCCAGTCAACTGCAAGCGAT |
| | GCAGCGGGTGCCGATGCCCAAGCGGGGGCACGGCAACTCTACTCAGCCCTCGCGAA |
| | TAAATGTTGTCATGTAGGGTGTACTAAGAGAAGCCTCGCGCAATTTTGT |
| 214 | ATGGAAACTGATACTCTTCTTTTGTGGGTACTGTTGTTGTGGGTCCCAGGAAGTAC |
| | CGGCGATAAAACGCATACTTGCCCGCCGTGCCCAGCACCTGAGGCAGCCGGCGGCC |
| | CTAGTGTCTTCTTGTTCCCGCCCAAGCCCAAGGATACACTCATGATCTCCCGAACG |
| | CCAGAGGTCACATGCGTAGTTGTTGACGTTTCCCATGAGGACCCTGAAGTGAAATT |
| | TAACTGGTACGTCGACGGCGTTGAGGTTCACAACGCTAAGACTAAGCCAAGAGAGG |
| | AACAGTACAATTCAACTTATAGAGTGGTGTCTGTATTGACAGTTCTCCATCAGGAT |
| | TGGCTGAACGGAAAAGAATATAAGTGCAAGGTCTCAAATAAGGCGCTCGCTGCACC |
| | CATAGAAAAAACCATATCAAAAGCGAAGGGGCAACCAAGAGAACCCCAGGTGTACA |
| | CGCTCCCCCCGTCCAGAGATGAACTCACGAAGAATCAAGTGTCACTCACATGTCTT |
| | GTAAAGGGGTTCTACCCCTCTGATATTGCCGTAGAATGGGAAAGCAACGGACAGCC |
| | CGAGAATAACTACAAGACGACACCGCCAGTTCTTGATTCTGACGGAAGCTTTTTCC |
| | TCTATTCAAAATTGACCGTTGACAAGTCCCGATGGCAACAGGGCAACGTTTTCTCA |
| | TGCTCCGTCCTTCACGAAGCCTTGCATTCCCACTATACGCAGAAGAGTCTCTCTTT |
| | GAGCCCCGGAGAGGGAGGCAGTGATTCATGGCAAGAGGAAGTGATCAAACTTTGCG |
| | GCAGAGAATTGGTTAGAGCCCAGATTGCCATTTGTGGACAAAGTACGGCCTCAGAT |
| | GCTGCGGGGGCACAAGCTCAGGCGGGCGCACGCCAGTTGTACAGTGCTCTGGCGAA |
| | TAAGTGCTGCCACGTTGGTTGCACCAAGCGATCCTTGGCGCAATTTTGC |
| 215 | ATGGAAACAGATACGCTCCTTTTGTGGGTACTGTTGTTGTGGGTGCCCGGCTCTAC |
| | GGGCGACAAGACTCATACTTGTCCGCCCTGCCCCGCTCCTGAGGCTGCCGGAGGCC |
| | CTTCAGTATTCTTGTTTCCGCCGAAACCGAAGGATACCTTGATGATTAGTAGGACA |
| | CCGGAAGTCACCTGCGTAGTGGTGGACGTAAGCCACGAAGATCCCGAAGTAAAGTT |
| | TAATTGGTATGTTGATGGCGTAGAGGTGCATAATGCGAAAACCAAACCTAGGGAGG |
| | AACAGTACAATAGTACTTACCGCGTAGTGTCAGTGCTTACCGTGCTGCATCAGGAC |
| | TGGCTTAATGGGAAGGAATACAAATGTAAAGTATCCAATAAAGCGCTGGCGGCTCC |
| | CATCGAGAAAACGATCTCAAAAGCCAAAGGACAACCACGGGAACCGCAGGTCTATA |
| | CTCTGCCACCTTCAAGAGACGAACTTACCAAGAACCAAGTCTCATTGACGTGCTTG |
| | GTAAAAGGTTTTTATCCGTCTGACATCGCTGTTGAATGGGAGTCTAACGGCCAGCC |
| | GGAGAACAATTACAAAACAACTCCACCAGTCTTGGATTCAGATGGGTCTTTTTTTT |
| | TGTATTCAAAGCTTACCGTTGACAAAAGCCGCTGGCAACAAGGAAACGTTTTCAGC |
| | TGCAGTGTGCTGCACGAAGCGCTCCACAGTCATTATACCCAGAAATCTTTGAGCCT |
| | GTCTCCAGGGGAAGGTGGGAGTGACTCTTGGCAAGAAGAGGTTATCAAACTTTGCG |
| | GGCGGGAGCTGGTAAGGGCCCAAATTGCAATATGCGGCAAAAGTACTGCATCTGAT |
| | GCCGCTGGGGCCGATGCTAACGCGGGCGCAAGACAACTTTATAGCGCGTTGGCGAA |
| | CAAATGCTGTCATGTGGGATGCACCAAACAAAGTTTGGCGCAATTTTGT |
| 216 | ATGGAAACCGATACCTTGCTGCTTTGGGTGCTCTTGCTGTGGGTTCCCGGTTCCAC |
| | GGGTGATAAAACTCACACGTGTCCGCCATGCCCCGCACCTGAAGCGGCGGGTGGTC |
| | CGAGCGTGTTTTTGTTTCCGCCTAAGCCCAAGGATACCCTGATGATTAGTCGGACA |
| | CCCGAAGTAACATGTGTCGTCGTGGATGTAAGTCACGAGGATCCCGAAGTGAAATT |
| | CAACTGGTATGTGGATGGAGTTGAAGTCCATAATGCGAAAACAAAACCGAGAGAGG |
| | AACAGTACAACTCAACATACCGGGTGGTAAGTGTACTGACGGTACTCCACCAGGAC |
| | TGGCTGAATGGTAAGGAGTACAAATGCAAAGTTTCAAATAAGGCGCTCGCTGCCCC |
| | CATCGAGAAAACCATTAGTAAGGCTAAAGGTCAACCTAGGGAGCCACAAGTATATA |
| | CATTGCCGCCTTCTAGAGATGAGCTGACCAAAAACCAGGTCAGCCTGACCTGTTTG |
| | GTGAAAGGCTTCTATCCAAGCGACATTGCTGTCGAGTGGGAGTCAAATGGGCAGCC |
| | GGAAAATAACTATAAAACGACTCCTCCTGTTCTCGACTCCGATGGTTCATTCTTCC |
| | TCTACTCAAAGCTTACCGTGGATAAATCCAGGTGGCAACAAGGTAACGTGTTCTCA |
| | TGTTCCGTTCTGCACGAAGCACTGCATTCCCATTATACACAAAAATCCCTGAGCCT |
| | CTCACCTGGGGAGGGCGGAAGCGATAGTTGGCAAGAGGAAGTAATAAAGCTGTGTG |
| | GCAGGGAACTCGTAAGGGCTCAGATTGCGATATGTGGAAAAAGCACTGCTTCTGAC |
| | GCCGCAGGGGCCAACGCAGATGCTGGCGCCCGACAACTCTATTCTGCGCTTGCGAA |
| | CAAGTGTTGTCATGTAGGATGTACCAAGCAAAGCCTTGCTCAGTTCTGT |
| 217 | ATGGAGACCGATACACTCTTGCTCTGGGTCCTTCTTCTTTGGGTTCCAGGCTCCAC |
| | AGGAGACAAAACCCACACTTGTCCGCCCTGTCCCGCTCCGGAGGCTGCAGGCGGCC |
| | CAAGTGTGTTTCTTTTCCCCCCAAAGCCGAAAGACACCTTGATGATATCCCGCACA |
| | CCCGAAGTGACTTGCGTTGTCGTCGACGTGTCTCATGAGGACCCAGAAGTCAAGTT |
| | TAATTGGTACGTTGATGGCGTGGAAGTTCACAATGCGAAAACTAAGCCCAGAGAGG |
| | AGCAATATAACTCAACCTACCGGGTGGTAAGTGTTCTGACAGTTCTCCACCAGGAC |
| | TGGTTGAACGGAAAAGAATACAAATGCAAAGTGAGTAACAAAGCCCTGGCTGCCCC |
| | TATCGAAAAGACCATATCCAAAGCGAAGGGCCAGCCACGGGAACCGCAAGTATATA |
| | CACTTCCACCATCTAGAGATGAGCTTACAAAGAACCAGGTGTCCCTTACCTGCCTT |
| | GTCAAAGGCTTCTATCCCTCTGACATCGCAGTGGAGTGGGAGTCCAACGGACAACC |
| | AGAGAACAACTATAAGACAACGCCGCCAGTACTGGATTCAGATGGTTCATTCTTCT |
| | TGTATTCTAAACTGACTGTTGATAAATCCCGATGGCAGCAGGGCAACGTTTTTAGT |
| | TGTAGTGTTCTGCACGAAGCCCTTCATTCCCATTATACACAAAAATCTCTTTCCCT |
| | CAGCCCAGGCGAGGGAGGAAGTGACAGTTGGCAAGAGGAGGTGATAAAGCTCTGTG |
| | GGAGGGAGCTGGTACGCGCACAGATTGCAATCTGCGGAAAGAGCACAGCAAGCGAT |
| | GCTGCTGGGGCCGATGCCGATGCTGGCGCTCGACAATTGTATTCAGCTCTTGCTAA |
| | CAAATGCTGTCACGTAGGATGCACTAAACAGAGCCTTGCTCAATTTTGT |
| 218 | ATGGAAACCGACACTCTTCTGCTGTGGGTTCTTCTCCTGTGGGTGCCTGGATCAAC |
| | CGGAGATAAGACCCACACATGTCCACCATGCCCAGCCCCAGAAGCGGCAGGTGGTC |
| | CTTCTGTGTTTCTCTTTCCTCCCAAACCGAAAGATACTCTGATGATAAGCCGGACC |
| | CCAGAAGTTACGTGCGTTGTAGTAGACGTGTCTCACGAGGACCCAGAAGTGAAGTT |
| | TAACTGGTATGTCGACGGTGTAGAAGTTCATAATGCGAAAACAAAGCCCAGGGAAG |
| | AACAATATAATTCAACGTACCGGGTCGTTTCCGTGCTGACAGTTCTGCACCAAGAT |
| | TGGCTCAACGGGAAAGAGTACAAATGCAAAGTATCAAATAAGGCCTTGGCTGCGCC |
| | GATTGAAAAGACGATTTCCAAAGCAAAGGGCCAGCCAAGGGAACCCCAGGTCTATA |
| | CCCTCCCTCCTAGCAGAGATGAACTTACAAAAAACCAAGTCTCCCTCACCTGCCTG |
| | GTCAAAGGATTCTATCCCTCAGATATAGCAGTAGAATGGGAAAGTAACGGGCAGCC |
| | CGAAAACAATTATAAGACCACTCCTCCAGTACTCGATTCAGACGGTAGCTTCTTTC |
| | TGTATTCCAAGCTGACCGTAGATAAAAGTAGGTGGCAGCAAGGTAATGTCTTCTCA |
| | TGTAGTGTACTTCATGAGGCGTTGCATTCCCATTACACGCAAAAGTCTTTGAGTCT |
| | CAGTCCGGGTGAAGGAGGTAGCGATTCTTGGCAGGAAGAAGTAATTAAGCTGTGCG |
| | GCCGGGAGCTCGTCAGGGCTCAGATAGCTATATGCGGCAAGAGCACGGCCAGTGAT |
| | GCTGCTGGTGCAGAGGCTGAAGCAGGTGCCAGGCAGTTGTACAGCGCACTCGCTAA |
| | TAAGTGTTGCCACGTGGGGTGTACAAAGCAATCTTTGGCACAATTCTGT |
| 219 | ATGGAGACTGATACTTTGCTGCTGTGGGTTCTCCTTCTTTGGGTCCCAGGTTCCAC |
| | AGGAGATAAGACCCATACTTGTCCTCCGTGCCCGGCACCAGAGGCTGCGGGTGGCC |
| | CATCCGTTTTCCTGTTTCCGCCAAAGCCTAAGGATACTCTGATGATTTCACGCACA |
| | CCCGAAGTGACCTGCGTGGTGGTCGACGTATCTCACGAAGACCCAGAGGTAAAATT |
| | CAATTGGTACGTGGACGGCGTCGAGGTTCATAACGCGAAAACTAAGCCGAGAGAAG |
| | AGCAGTACAACTCTACGTATCGCGTGGTGTCCGTACTGACAGTATTGCATCAGGAC |
| | TGGTTGAATGGCAAGGAGTATAAGTGCAAGGTATCTAATAAGGCATTGGCTGCCCC |
| | AATAGAGAAAACGATCAGCAAAGCAAAGGGGCAGCCGCGCGAGCCGCAGGTATATA |
| | CACTTCCACCATCACGGGATGAGTTGACGAAAAATCAAGTCTCTCTCACATGTCTG |
| | GTAAAAGGTTTCTATCCTTCTGATATCGCCGTGGAATGGGAAAGCAACGGCCAACC |
| | CGAAAACAACTATAAGACGACGCCGCCGGTACTCGACAGCGACGGAAGCTTTTTCT |
| | TGTATTCCAAGTTGACAGTGGACAAGTCTCGATGGCAGCAAGGAAACGTGTTCTCA |
| | TGTTCTGTTCTTCACGAAGCCCTTCATAGCCATTATACTCAGAAATCTCTCTCACT |
| | CTCCCCAGGTGAAGGGGGAAGTGACTCTTGGCAAGAAGAAGTCATTAAGCTTTGCG |
| | GTCGAGAATTGGTTCGGGCTCAAATAGCTATTTGTGGCAAGTCCACGGCAAGTGAT |
| | GCAGCGGGGGCTCAGGCAGACGCGGGCGCAAGGCAGCTTTATTCCGCACTTGCAAA |
| | TAAGTGCTGTCACGTCGGATGTACTAAACAATCACTTGCACAATTCTGC |
| 220 | ATGGAGACCGACACACTTCTCCTCTGGGTCCTGCTCCTTTGGGTGCCAGGCAGTAC |
| | AGGTGACAAGACACATACATGTCCCCCATGCCCTGCACCCGAAGCTGCTGGGGGGC |
| | CCAGCGTGTTCCTGTTTCCGCCGAAGCCCAAGGACACATTGATGATTAGTAGAACC |
| | CCAGAGGTAACTTGTGTTGTGGTCGATGTGTCACATGAAGACCCCGAGGTAAAGTT |
| | TAACTGGTATGTGGATGGGGTAGAGGTACATAATGCAAAAACCAAGCCGCGGGAGG |
| | AGCAGTATAATTCAACCTATCGAGTCGTGTCAGTCTTGACCGTGCTCCACCAGGAC |
| | TGGCTTAACGGTAAGGAGTATAAATGCAAAGTCAGTAATAAGGCATTGGCCGCCCC |
| | CATTGAGAAGACCATCAGTAAAGCTAAGGGGCAACCTAGAGAGCCACAGGTTTACA |
| | CCCTCCCTCCCTCCCGGGATGAACTCACCAAAAACCAGGTGTCCCTTACTTGTTTG |
| | GTAAAGGGCTTTTATCCTTCTGATATTGCTGTTGAATGGGAGTCTAACGGGCAACC |
| | TGAAAATAACTACAAAACAACTCCCCCCGTTCTGGACTCTGATGGGTCATTCTTCC |
| | TTTATTCAAAATTGACAGTTGATAAGAGTAGATGGCAACAAGGCAACGTATTTTCA |
| | TGTTCTGTGCTCCACGAGGCTCTCCATTCCCACTACACACAGAAAAGTCTCTCACT |
| | GTCCCCAGGAGAGGGCGGGAGCGACTCTTGGCAGGAAGAAGTAATCAAGTTGTGTG |
| | GCAGGGAACTCGTACGCGCTCAGATTGCAATATGCGGGAAATCCACGGCAAGTGAC |
| | GCTGCCGGGGCCGACGCGCAAGCAGGGGCACGGCAGCTTTACTCCGCCCTCGCAAA |
| | TAAATGTTGTCATGTGGGATGCACTAAACAGTCCCTTGCCCAGTTTTGC |
| 221 | ATGGAGACAGATACACTGCTTCTGTGGGTGCTCTTGCTTTGGGTCCCCGGTTCCAC |
| | AGGCGATAAAACCCATACCTGTCCACCATGCCCCGCGCCAGAGGCAGCGGGTGGTC |
| | CAAGCGTTTTCCTTTTTCCACCGAAACCAAAAGATACACTTATGATATCAAGGACC |
| | CCCGAGGTAACGTGCGTCGTAGTTGACGTTTCTCACGAAGATCCCGAGGTGAAATT |
| | CAATTGGTACGTAGATGGTGTAGAGGTACACAATGCGAAGACAAAACCGCGGGAAG |
| | AGCAGTATAATAGCACATACAGAGTCGTGAGCGTCCTCACCGTACTTCACCAAGAT |
| | TGGCTGAATGGAAAGGAGTACAAATGTAAGGTAAGTAATAAAGCACTTGCGGCCCC |
| | CATCGAGAAAACTATCAGTAAAGCAAAAGGGCAACCACGAGAACCCCAGGTCTACA |
| | CTTTGCCACCATCACGGGATGAACTGACAAAAAATCAGGTGTCACTCACTTGCCTT |
| | GTTAAAGGGTTCTATCCTAGTGACATAGCGGTAGAATGGGAGTCTAACGGGCAGCC |
| | TGAGAACAATTATAAAACTACGCCCCCTGTTCTTGATTCCGATGGATCATTTTTTC |
| | TCTACTCCAAACTCACCGTAGACAAATCCCGCTGGCAGCAGGGCAACGTGTTTAGT |
| | TGCAGCGTTCTTCACGAAGCACTTCACTCACATTACACACAAAAGTCCCTGAGCTT |
| | GAGTCCTGGGGAGGGTGGATCTGATTCTTGGCAGGAAGAAGTTATAAAACTTTGTG |
| | GCAGAGAGTTGGTCCGCGCACAAATCGCCATATGTGGTAAAAGCACAGCGTCTGAC |
| | GCGGCGGGAGCGCAAGCCCAGGCGGGGGCTCGGCAACTCTACTCAGCCCTGGCTAA |
| | CAAGTGCTGTCACGTGGGATGCACTAAACAAAGTCTGGCGCAGTTCTGC |
| 222 | ATGGAGACCGATACGTTGCTGTTGTGGGTTTTGTTGCTGTGGGTACCTGGATCTAC |
| | AGGTGACAAAACCCATACGTGTCCCCCGTGTCCGGCTCCAGAGGCTGCGGGAGGAC |
| | CGTCTGTGTTCTTGTTCCCGCCGAAGCCTAAAGATACGCTGATGATTAGTCGGACC |
| | CCCGAGGTGACCTGCGTGGTAGTAGACGTATCTCATGAAGATCCGGAAGTAAAGTT |
| | TAACTGGTACGTAGACGGCGTCGAGGTACATAATGCCAAGACGAAACCCAGAGAAG |
| | AGCAATATAATAGCACTTATCGAGTTGTAAGCGTATTGACGGTCCTTCACCAGGAC |
| | TGGTTGAACGGCAAAGAGTACAAATGTAAGGTATCCAATAAAGCATTGGCTGCGCC |
| | AATTGAAAAGACAATTTCCAAAGCGAAGGGGCAACCTCGAGAGCCGCAAGTCTACA |
| | CGCTGCCACCGAGTAGGGATGAATTGACTAAGAATCAGGTGAGTCTCACGTGTCTC |
| | GTGAAGGGGTTTTACCCCAGTGATATTGCGGTAGAATGGGAGTCCAACGGTCAGCC |
| | AGAAAATAATTATAAAACAACGCCCCCTGTATTGGATTCTGACGGGAGCTTTTTCC |
| | TGTACTCAAAACTCACCGTAGATAAGAGTCGCTGGCAACAGGGCAACGTATTCTCA |
| | TGTAGCGTTCTGCACGAGGCGCTGCACTCTCACTACACACAGAAGAGTTTGAGTTT |
| | GTCCCCTGGCGAAGGAGGTTCTGATTCCTGGCAGGAGGAGGTGATTAAGCTGTGTG |
| | GCCGCGAATTGGTGAGGGCTCAAATTGCTATTTGCGGACAGAGCACAGCGTCCGAT |
| | GCCGCCGGCGCAGATGCTAATGCCGGTGCAAGGCAACTGTACTCCGCTCTCGCCAA |
| | TAAGTGTTGTCATGTCGGCTGCACCAAGCAATCCCTGGCCCAGTTTTGC |
| 223 | ATGGAGACAGATACGCTCTTGCTGTGGGTACTCCTCCTCTGGGTCCCAGGCTCAAC |
| | GGGCGACAAGACTCACACTTGTCCCCCATGTCCAGCACCGGAAGCTGCCGGCGGTC |
| | CCTCAGTTTTCCTTTTCCCCCCCAAACCCAAGGACACCCTTATGATTTCAAGGACA |
| | CCAGAGGTAACGTGCGTAGTGGTGGACGTCAGTCATGAAGACCCAGAGGTAAAGTT |
| | TAACTGGTACGTGGATGGGGTAGAGGTTCATAATGCTAAAACAAAACCACGCGAGG |
| | AACAGTACAATAGTACGTATAGAGTGGTCTCCGTTCTTACGGTGCTGCATCAGGAC |
| | TGGCTGAACGGAAAAGAGTACAAGTGTAAGGTTAGCAATAAGGCGCTGGCGGCCCC |
| | AATCGAAAAGACGATTTCTAAGGCCAAAGGCCAGCCAAGGGAGCCACAAGTATATA |
| | CCCTTCCCCCTTCCCGAGATGAGCTGACTAAGAATCAAGTCAGTCTCACCTGCCTT |
| | GTCAAAGGGTTCTACCCATCCGATATTGCTGTTGAATGGGAGTCTAATGGCCAGCC |
| | GGAGAACAATTACAAGACAACTCCGCCTGTATTGGATTCCGACGGGTCTTTTTTCC |
| | TCTATTCAAAACTCACAGTAGACAAAAGTCGATGGCAGCAAGGTAACGTGTTTTCT |
| | TGCTCTGTGTTGCATGAAGCACTTCATTCTCATTATACTCAAAAATCATTGAGCCT |
| | CAGTCCAGGCGAAGGGGGTAGTGACTCATGGCAGGAGGAGGTAATCAAGCTTTGCG |
| | GACGAGAGTTGGTCAGGGCCCAGATAGCTATTTGTGGTCAGTCCACGGCGAGTGAC |
| | GCAGCAGGGGCGAATGCCGATGCAGGAGCAAGACAACTGTATTCTGCTCTGGCCAA |
| | CAAGTGTTGTCATGTAGGGTGTACTAAACAAAGTCTCGCCCAGTTCTGC |
| 224 | ATGGAAACGGATACGCTGTTGTTGTGGGTCTTGCTCCTGTGGGTCCCCGGATCCAC |
| | AGGTGATAAAACCCACACCTGTCCCCCATGTCCGGCTCCCGAAGCAGCGGGGGGCC |
| | CTTCAGTTTTTCTCTTTCCCCCCAAACCGAAAGACACGCTGATGATTAGCAGAACT |
| | CCAGAGGTTACCTGTGTAGTTGTGGACGTTTCACACGAGGATCCCGAGGTTAAATT |
| | CAACTGGTATGTGGACGGCGTCGAAGTGCATAATGCAAAAACAAAGCCCCGGGAAG |
| | AACAATATAATAGTACCTATAGGGTCGTTTCCGTACTGACCGTACTTCATCAAGAT |
| | TGGCTCAATGGGAAGGAATACAAATGTAAAGTGAGTAATAAAGCCCTGGCGGCACC |
| | GATCGAAAAAACCATTTCAAAGGCTAAGGGACAACCGCGCGAACCTCAGGTCTATA |
| | CCTTGCCCCCTTCACGCGACGAGCTTACGAAGAATCAGGTAAGCCTTACTTGTCTT |
| | GTCAAGGGTTTTTACCCCAGCGACATAGCTGTCGAGTGGGAATCCAATGGCCAACC |
| | GGAGAATAATTACAAAACTACCCCTCCTGTTCTTGATAGCGACGGAAGCTTCTTCT |
| | TGTATTCCAAACTCACAGTAGATAAAAGTAGGTGGCAGCAGGGCAATGTATTTTCT |
| | TGCAGCGTCCTGCATGAAGCACTGCATAGCCATTATACTCAAAAGTCCCTGTCTTT |
| | GTCTCCTGGAGAGGGCGGAAGCGATTCTTGGCAAGAGGAAGTTATTAAGCTGTGCG |
| | GGCGCGAACTTGTGAGGGCTCAAATAGCGATATGTGGTCAGAGCACCGCTAGCGAT |
| | GCGGCTGGTGCAGACGCCGATGCCGGTGCTAGGCAACTTTACAGTGCACTTGCGAA |
| | TAAGTGCTGTCACGTCGGATGTACTAAACAAAGCCTCGCCCAGTTCTGC |
| 225 | ATGGAAACGGATACTCTTCTTTTGTGGGTTCTTCTTCTGTGGGTACCAGGAAGTAC |
| | TGGTGATAAAACACATACTTGCCCTCCTTGTCCGGCTCCCGAAGCCGCAGGTGGAC |
| | CTTCCGTCTTTCTTTTCCCACCCAAACCTAAAGACACTTTGATGATTAGCCGGACC |
| | CCCGAGGTAACCTGTGTCGTAGTTGACGTTTCCCATGAAGACCCCGAAGTTAAGTT |
| | CAACTGGTATGTCGACGGCGTCGAGGTGCACAACGCGAAGACTAAGCCAAGAGAGG |
| | AGCAATACAATTCAACTTACAGGGTCGTGTCCGTCTTGACAGTGCTTCATCAAGAC |
| | TGGCTTAATGGAAAGGAATACAAATGTAAAGTCTCCAACAAGGCTCTCGCAGCGCC |
| | CATTGAGAAAACGATATCCAAAGCGAAGGGTCAACCAAGAGAACCCCAGGTTTACA |
| | CCCTCCCCCCTAGTCGGGACGAGCTTACGAAGAACCAGGTCAGTTTGACATGCCTG |
| | GTGAAAGGCTTCTATCCGTCAGACATCGCCGTAGAGTGGGAAAGCAACGGGCAACC |
| | CGAGAACAACTATAAGACGACTCCCCCGGTGTTGGATAGCGATGGCTCTTTCTTCC |
| | TGTACTCTAAGCTGACCGTAGATAAATCCAGGTGGCAACAGGGGAACGTGTTTTCA |
| | TGCTCAGTGCTCCATGAAGCCCTCCATTCACACTATACACAAAAGTCTTTGTCACT |
| | GTCCCCCGGTGAAGGCGGCAGTGATAGCTGGCAAGAAGAAGTCATAAAGCTCTGTG |
| | GTCGCGAGCTTGTTAGGGCCCAAATTGCGATCTGTGGTCAGTCAACGGCTTCTGAC |
| | GCCGCCGGAGCGGAAGCCGAGGCGGGTGCTCGGCAATTGTATTCAGCACTGGCGAA |
| | CAAATGTTGCCATGTTGGTTGTACTAAACAAAGCCTGGCCCAGTTTTGC |
| 226 | ATGGAGACCGATACCTTGTTGCTGTGGGTGCTTCTTCTCTGGGTTCCGGGATCTAC |
| | AGGAGACAAGACTCACACTTGTCCACCCTGTCCAGCACCTGAAGCCGCTGGTGGAC |
| | CATCTGTCTTTCTGTTCCCCCCTAAACCAAAGGATACACTTATGATCAGCAGAACA |
| | CCTGAAGTCACATGCGTTGTGGTAGACGTTTCCCACGAGGATCCTGAAGTGAAGTT |
| | TAACTGGTACGTGGATGGCGTTGAGGTTCATAATGCCAAGACGAAACCTCGGGAGG |
| | AGCAGTATAATTCTACTTATAGGGTGGTAAGCGTACTGACAGTCCTCCATCAAGAC |
| | TGGTTGAACGGGAAGGAATACAAGTGTAAAGTTTCCAACAAAGCTCTGGCGGCGCC |
| | TATAGAAAAGACAATATCAAAAGCGAAAGGGCAACCCAGAGAGCCTCAAGTATATA |
| | CATTGCCCCCTAGCAGAGACGAATTGACGAAAAATCAGGTCTCTCTCACGTGCCTC |
| | GTGAAGGGCTTCTATCCTAGTGATATAGCTGTGGAATGGGAATCCAATGGACAGCC |
| | AGAAAACAACTACAAGACCACGCCCCCCGTCTTGGATTCCGACGGGTCATTCTTCC |
| | TGTACAGCAAGCTGACTGTCGACAAGAGTCGATGGCAACAGGGCAACGTCTTTAGC |
| | TGCAGCGTCCTGCACGAAGCTCTGCATAGTCATTACACCCAAAAGTCCCTTTCTCT |
| | CTCCCCTGGTGAAGGCGGTTCCGATTCATGGCAAGAAGAAGTAATTAAGCTCTGTG |
| | GACGAGAGCTTGTCCGAGCACAAATTGCGATCTGCGGGCAGAGTACCGCATCTGAC |
| | GCTGCTGGCGCGCAGGCAGATGCGGGTGCACGGCAGCTTTATTCAGCTCTCGCCAA |
| | CAAGTGTTGTCATGTGGGGTGTACAAAGCAGAGCCTTGCCCAGTTTTGT |
| 227 | ATGGAAACGGACACACTGCTGCTTTGGGTTCTCTTGCTGTGGGTCCCAGGCTCTAC |
| | AGGGGATAAGACCCATACGTGTCCCCCTTGCCCTGCACCCGAGGCGGCTGGGGGCC |
| | CTTCCGTATTCTTGTTTCCTCCTAAGCCCAAAGATACCTTGATGATAAGTCGAACG |
| | CCAGAAGTGACTTGCGTTGTTGTGGATGTCTCCCACGAGGATCCAGAAGTCAAATT |
| | TAACTGGTATGTCGATGGGGTCGAAGTGCATAATGCTAAAACGAAACCCAGAGAGG |
| | AACAATACAATTCAACATACCGCGTAGTCAGTGTTCTTACTGTGCTCCATCAGGAT |
| | TGGCTCAATGGGAAAGAATACAAGTGTAAAGTCTCAAATAAAGCATTGGCGGCCCC |
| | TATAGAGAAGACCATAAGCAAGGCTAAAGGTCAGCCTAGGGAGCCTCAAGTATATA |
| | CCTTGCCTCCTAGCAGAGATGAGTTGACCAAGAACCAGGTCAGCCTCACATGCCTG |
| | GTGAAAGGGTTTTACCCATCTGATATTGCCGTCGAGTGGGAAAGTAATGGGCAGCC |
| | AGAGAACAACTACAAGACGACACCACCGGTACTGGATAGTGACGGAAGTTTTTTTC |
| | TTTACAGTAAGCTCACAGTCGACAAAAGCCGGTGGCAACAAGGAAATGTATTTTCA |
| | TGTAGCGTACTTCATGAAGCCCTCCACTCTCATTACACGCAGAAGTCACTTTCACT |
| | TAGTCCGGGTGAGGGTGGAAGCGATAGCTGGCAAGAGGAGGTTATCAAGCTCTGTG |
| | GACGAGAACTCGTGAGAGCGCAAATTGCAATCTGCGGGCAGAGCACGGCGAGTGAT |
| | GCGGCCGGGGCGGACGCGCAAGCAGGAGCACGACAACTTTATAGTGCTTTGGCTAA |
| | TAAATGTTGCCACGTTGGATGTACTAAACAGAGCTTGGCACAGTTTTGC |
| 228 | ATGGAGACTGATACACTGCTTCTGTGGGTATTGTTGTTGTGGGTCCCCGGTTCCAC |
| | AGGTGATAAAACGCACACTTGTCCGCCATGTCCGGCACCTGAGGCAGCGGGAGGAC |
| | CGTCCGTGTTTCTGTTTCCCCCTAAACCAAAGGACACGCTGATGATCAGCCGAACA |
| | CCTGAAGTAACATGCGTGGTCGTTGACGTGTCTCACGAGGATCCAGAAGTAAAGTT |
| | CAATTGGTATGTTGACGGAGTTGAAGTACATAATGCTAAGACTAAACCCCGCGAAG |
| | AACAATATAATTCTACGTACAGAGTTGTATCCGTGCTCACGGTACTTCACCAAGAT |
| | TGGCTTAACGGGAAAGAATATAAGTGTAAGGTCTCAAATAAGGCCCTGGCTGCTCC |
| | GATCGAAAAAACGATATCAAAGGCAAAGGGTCAACCTCGGGAGCCTCAAGTATATA |
| | CCCTCCCCCCATCTAGGGATGAGCTGACAAAGAACCAAGTTTCACTGACCTGTCTC |
| | GTAAAGGGTTTCTATCCTTCTGACATCGCAGTTGAATGGGAGTCCAACGGCCAACC |
| | AGAGAACAACTATAAGACGACACCCCCCGTGTTGGACAGTGACGGAAGTTTTTTCC |
| | TGTACTCCAAGCTGACGGTTGATAAAAGTAGATGGCAACAAGGAAATGTTTTCAGT |
| | TGTTCTGTGTTGCACGAGGCCCTCCACTCACACTATACCCAAAAAAGTTTGTCTCT |
| | GAGTCCCGGTGAAGGCGGGAGCGATTCATGGCAGGAGGAAGTAATCAAACTTTGTG |
| | GGCGAGAACTGGTCAGGGCGCAAATAGCGATATGTGGGCAAAGCACAGCTTCAGAT |
| | GCAGCCGGTGCTCAAGCTCAGGCTGGAGCTCGACAGCTTTATAGCGCCTTGGCTAA |
| | TAAATGTTGTCACGTTGGCTGTACGAAGCAGAGCCTGGCACAGTTCTGC |
| 229 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTCACGTTGGGTGTACCAAGCGGTCCCTTGCGCAATTCTGC |
| 230 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATTTCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTCACGTTGGGTGTACCAAGCGGTCCCTTGCGCAATTCTGC |
| 231 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCACTTCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTCACGTTGGGTGTACCAAGCGGTCCCTTGCGCAATTCTGC |
| 232 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCAATTCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTCACGTTGGGTGTACCAAGCGGTCCCTTGCGCAATTCTGC |
| 233 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATGGCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTTACGTTGGGTGTACCAAGCGGTCCCTTGCGCAATTCTGC |
| 234 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATGGCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTCTTGTTGGGTGTACCAAGCGGTCCCTTGCGCAATTCTGC |
| 235 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATGGCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTCAAGTTGGGTGTACCAAGCGGTCCCTTGCGCAATTCTGC |
| 236 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATGGCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTAAAGTTGGGTGTACCAAGCGGTCCCTTGCGCAATTCTGC |
| 237 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTGAAGGCGGAAGCGACTCATACCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTTACGTTGGGTGTACCAAGCGGTCCCTTGCGCAATTCTGC |
| 238 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTGAAGGCGGAAGCGACTCATACCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTAAAGTTGGGTGTACCAAGCGGTCCCTTGCGCAATTCTGC |
| 239 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTGAAGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGAGAAGGGGGCGGAAGACAGCTTTACTCTGCTCTGGCTAA |
| | CAAGTGTTGTTACGTTGGGTGTACCAAGCGGTCCCTTGCGCAATTCTGC |
| 240 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTGAAGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGAGAAGGGGGCGGAAGACAGCTTTACTCTGCTCTGGCTAA |
| | CAAGTGTTGTAAAGTTGGGTGTACCAAGCGGTCCCTTGCGCAATTCTGC |
| 241 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTGAAGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGATCTGGGGGCGGAAGACAGCTTTACTCTGCTCTGGCTAA |
| | CAAGTGTTGTTACGTTGGGTGTACCAAGCGGTCCCTTGCGCAATTCTGC |
| 242 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTGAAGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGATCTGGGGGCGGAAGACAGCTTTACTCTGCTCTGGCTAA |
| | CAAGTGTTGTAAAGTTGGGTGTACCAAGCGGTCCCTTGCGCAATTCTGC |
| 243 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTTACGTTGGGTGTACCAAGCAATCCCTTGCGCAATTCTGC |
| 244 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGCTAGTGAC |
| | GCAGCGGGCGCCGAAGCGGAAGCTGGTGCAAGGCAGCTTTACTCTGCTCTGGCTAA |
| | CAAGTGTTGTAAAGTTGGGTGTACCAAGCAATCCCTTGCGCAATTCTGC |
| 245 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGAGAAGGGGGCGGAAGACAGCTTTACTCTGCTCTGGCTAA |
| | CAAGTGTTGTTACGTTGGGTGTACCAAGCAATCCCTTGCGCAATTCTGC |
| 246 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGAGAAGGGGGCGGAAGACAGCTTTACTCTGCTCTGGCTAA |
| | CAAGTGTTGTAAAGTTGGGTGTACCAAGCAATCCCTTGCGCAATTCTGC |
| 247 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGATCTGGGGGCGGAAGACAGCTTTACTCTGCTCTGGCTAA |
| | CAAGTGTTGTTACGTTGGGTGTACCAAGCAATCCCTTGCGCAATTCTGC |
| 248 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGATCTGGGGGCGGAAGACAGCTTTACTCTGCTCTGGCTAA |
| | CAAGTGTTGTAAAGTTGGGTGTACCAAGCAATCCCTTGCGCAATTCTGC |
| 381 | ATGGAGACGGACACTTTGCTGCTTTGGGTACTGCTGCTTTGGGTTCCTGGATCTAC |
| | TGGCGATAAAACACACACGTGTCCCCCCTGCCCGGCTCCAGAGGCGGCTGGTGGTC |
| | CCAGCGTATTCTTGTTTCCTCCCAAACCTAAGGATACGCTCATGATATCCCGCACC |
| | CCAGAAGTTACGTGTGTAGTCGTCGACGTCAGTCACGAAGATCCAGAGGTCAAATT |
| | TAACTGGTATGTCGACGGAGTAGAGGTCCACAATGCGAAAACCAAGCCCAGAGAAG |
| | AGCAGTACAACTCCACGTATCGCGTCGTCTCCGTCCTCACCGTACTCCATCAAGAT |
| | TGGCTGAATGGGAAAGAGTATAAATGCAAAGTATCTAACAAGGCTCTGCCAGCTCC |
| | GATAGAAAAGACTATATCAAAGGCCAAGGGGCAGCCAAGGGAGCCTCAAGTCTATA |
| | CTTTGCCCCCATCTCGGGATGAGCTTACGAAAAACCAGGTCAGCCTTACCTGTCTT |
| | GTTAAAGGTTTTTATCCGAGTGACATCGCAGTGGAATGGGAATCTAATGGTCAACC |
| | TGAAAACAATTACAAAACCACACCGCCAGTATTGGACAGCGATGGTAGTTTTTTTC |
| | TTTACTCAAAACTGACTGTAGATAAAAGCAGATGGCAGCAGGGCAATGTCTTTTCA |
| | TGTAGCGTTATGCATGAGGCTCTTCACAACCACTATACCCAAAAGTCATTGTCTCT |
| | TAGTCCCGGAAAGGGCGGAAGTGATTCTTGGAAGGAGGAGGTAATCAAGTTGTGCG |
| | GGCGAGAGTTGGTACGGGCACAGATCGCGATATGCGGAAAATCCACAGGTGGGGGC |
| | GAAGGAGGAGGTGAGGGTGGAGGTGAAGGACGACAGTTGTATTCCGCCTTGGCAAA |
| | CAAGTGTTGCCATGTGGGTTGCACAAAACGCAGTCTTGCCCGCTTCTGT |
| 382 | ATGGAGACCGATACGCTGTTGCTGTGGGTATTGCTTCTCTGGGTGCCCGGCTCAAC |
| | TGGGGATAAGACACATACATGCCCTCCCTGTCCGGCTCCAGAGGCAGCCGGGGGTC |
| | CATCAGTCTTCCTTTTTCCGCCTAAACCTAAGGATACACTGATGATCTCTCGAACA |
| | CCGGAGGTCACTTGTGTTGTCGTTGACGTATCACATGAGGATCCCGAAGTAAAGTT |
| | CAACTGGTATGTCGATGGTGTGGAGGTTCATAATGCTAAAACTAAACCACGGGAGG |
| | AGCAATATAATTCCACATATAGGGTCGTGAGCGTGTTGACGGTGCTTCATCAAGAC |
| | TGGCTTAATGGGAAGGAATATAAATGCAAAGTGTCAAATAAAGCACTTCCTGCGCC |
| | AATCGAGAAAACAATTAGTAAGGCAAAGGGGCAGCCGCGAGAACCTCAGGTGTACA |
| | CCTTGCCGCCTTCTAGAGACGAGCTCACAAAGAACCAAGTTTCCCTGACTTGCCTC |
| | GTTAAGGGGTTTTATCCGTCCGATATAGCCGTGGAGTGGGAGTCAAACGGCCAACC |
| | GGAAAATAATTACAAAACGACACCCCCAGTATTGGATAGTGACGGCTCTTTTTTCC |
| | TTTATTCTAAGCTGACTGTGGACAAAAGCCGCTGGCAGCAGGGCAATGTCTTTTCA |
| | TGCAGCGTAATGCATGAAGCCCTGCACAACCACTACACGCAAAAATCCCTTTCCTT |
| | GTCACCCGGCAAGGGCGGCTCTGACTCCTGGAAAGAGGAAGTTATAAAACTCTGTG |
| | GCCGAGAACTTGTTCGAGCTCAAATCGCGATTTGTGGTAAGTCAACGGGTGGGGGC |
| | GAAGGTGGAGGCGAGGGTGGGGGAGAAGGAGGAGGCCAGTTGTACTCAGCTCTTGC |
| | AAATAAGTGTTGCCACGTTGGTTGTACGAAGCGGAGCCTTGCTCGCTTCTGC |
| 383 | ATGGAAACTGATACTCTTCTGCTGTGGGTCCTGCTGCTGTGGGTTCCAGGATCTAC |
| | TGGAGACAAAACACATACTTGTCCGCCTTGCCCGGCACCCGAAGCGGCCGGCGGAC |
| | CCAGTGTCTTTCTCTTCCCACCCAAACCGAAAGACACTCTGATGATTTCCAGGACG |
| | CCTGAAGTGACCTGCGTTGTAGTTGATGTATCACACGAGGATCCCGAGGTCAAGTT |
| | CAATTGGTATGTAGATGGGGTGGAGGTCCATAATGCAAAGACGAAGCCACGGGAGG |
| | AACAGTACAACTCTACGTACAGAGTTGTCAGTGTTTTGACCGTCCTTCATCAGGAT |
| | TGGCTGAACGGTAAAGAATATAAATGCAAGGTTAGCAATAAAGCTTTGCCCGCCCC |
| | TATAGAGAAAACGATCAGTAAGGCGAAGGGGCAGCCTAGGGAACCCCAGGTATATA |
| | CCTTGCCGCCAAGTCGAGATGAGCTGACGAAGAACCAAGTGAGTCTGACATGCCTC |
| | GTGAAGGGCTTCTATCCGAGCGATATCGCTGTCGAATGGGAGAGCAATGGGCAGCC |
| | TGAGAATAACTATAAAACAACGCCACCCGTCCTCGACTCCGATGGCTCATTCTTCC |
| | TGTACAGTAAACTTACAGTAGATAAGAGTAGATGGCAGCAGGGTAACGTCTTTAGT |
| | TGCTCCGTGATGCACGAGGCATTGCACAATCATTACACTCAAAAATCTCTGTCCCT |
| | GAGTCCGGGCAAAGGCGGTTCAGATAGCTGGATGGAGGAGGTCATAAAGCTTTGTG |
| | GACGAGAACTCGTTCGCGCCCAGATAGCTATTTGTGGGAAATCAACCGGGGGTGGA |
| | GAAGGTGGCGGAGAAGGGGGAGGCGAAGGGCGCCAACTGTATTCTGCATTGGCTAA |
| | TAAGTGCTGTCACGTAGGATGTACAAAAAGGTCTCTGGCGAGATTCTGC |
| 384 | ATGGAGACCGACACCCTCTTGTTGTGGGTTCTCCTCTTGTGGGTGCCCGGCAGTAC |
| | TGGAGACAAGACGCACACTTGTCCACCTTGCCCTGCGCCGGAAGCTGCTGGAGGCC |
| | CCAGTGTCTTTTTGTTCCCGCCCAAACCGAAGGACACTTTGATGATAAGTCGCACG |
| | CCCGAGGTTACCTGTGTGGTTGTCGATGTCTCACACGAAGATCCGGAGGTGAAGTT |
| | TAATTGGTATGTAGATGGCGTGGAGGTTCATAACGCCAAAACGAAACCCAGAGAAG |
| | AACAATATAACAGTACATATCGAGTAGTATCCGTTCTCACTGTCCTGCATCAAGAC |
| | TGGTTGAACGGGAAGGAATATAAGTGCAAGGTGAGCAATAAAGCACTCCCGGCCCC |
| | AATCGAAAAGACCATCAGCAAAGCGAAGGGGCAACCTCGAGAACCCCAGGTATATA |
| | CGCTCCCCCCTAGTCGGGATGAACTTACTAAAAATCAGGTTAGCCTCACTTGCCTT |
| | GTTAAAGGGTTCTATCCCAGTGATATTGCCGTCGAATGGGAATCAAACGGGCAGCC |
| | GGAAAATAACTACAAGACAACCCCTCCTGTGCTCGATAGCGATGGCTCTTTTTTCC |
| | TCTACAGCAAACTTACCGTTGATAAGAGCCGGTGGCAACAAGGTAATGTTTTCTCC |
| | TGCTCCGTTATGCATGAAGCACTCCATAACCATTATACCCAAAAAAGCCTGTCACT |
| | TAGTCCGGGTAAAGGAGGTAGTGATTCTTGGCAGGAGGAGGTAATCAAACTTTGTG |
| | GGAGGGAGCTGGTACGAGCTCAGATTGCTATATGTGGAAAAAGCACGGGCGGAGGA |
| | GAAGGAGGTGGCGAAGGCGGGGGTGAAGGTCGGCAACTCTACTCCGCTCTCGCTAA |
| | TAAGTGCTGCCACGTCGGGTGTACGAAGCGCTCCCTGGCGCGATTCTGC |
| 385 | ATGGAAACAGATACCCTCCTCCTCTGGGTCCTTCTTCTTTGGGTGCCTGGCTCAAC |
| | TGGAGATAAAACGCACACGTGTCCGCCCTGCCCAGCGCCTGAAGCCGCAGGCGGGC |
| | CGTCCGTCTTCCTCTTTCCTCCAAAACCCAAAGACACACTTATGATCAGTAGGACC |
| | CCAGAGGTAACCTGCGTCGTGGTCGACGTTTCCCATGAAGACCCAGAGGTCAAGTT |
| | CAACTGGTACGTCGACGGTGTCGAAGTACATAATGCTAAAACGAAGCCTCGGGAAG |
| | AGCAGTACAACTCTACCTACCGCGTCGTTTCCGTACTCACCGTACTTCACCAGGAC |
| | TGGCTTAACGGTAAAGAGTATAAATGCAAAGTATCTAATAAGGCTCTCGCCGCGCC |
| | GATTGAGAAGACAATTTCAAAGGCCAAGGGGCAGCCGCGGGAGCCCCAAGTGTATA |
| | CCTTGCCCCCGTCCCGAGATGAGCTGACTAAAAACCAAGTAAGCTTGACTTGCTTG |
| | GTCAAAGGCTTCTACCCTTCCGATATAGCTGTCGAATGGGAGTCAAATGGCCAACC |
| | AGAGAACAATTATAAAACTACACCCCCGGTCTTGGATTCTGATGGCTCATTTTTTC |
| | TCTATTCTAAACTGACCGTGGATAAGTCTCGCTGGCAGCAAGGTAACGTGTTCAGT |
| | TGCTCTGTTCTTCACGAAGCACTGCACAGTCATTACACTCAGAAGAGTCTTAGCCT |
| | GAGCCCTGGTAAAGGGGGTTCTGATTCCTGGCAGGAGGAAGTAATAAAACTCTGTG |
| | GCCGGGAGTTGGTACGGGCGCAGATTGCGATATGCGGTAAGAGCACCGGCGGAGGC |
| | GAAGGCGGTGGGGAAGGAGGAGGAGAAGGGAGACAACTCTATTCCGCATTGGCAAA |
| | TAAGTGCTGCCACGTCGGGTGTACCAAACGATCCCTTGCACGGTTCTGT |
| 386 | ATGGAGACGGACACCCTCCTTCTCTGGGTTTTGCTCCTTTGGGTCCCTGGTTCCAC |
| | TGGAGATAAGACCCATACGTGCCCCCCTTGCCCTGCGCCTGAGGCAGCGGGTGGCC |
| | CATCAGTCTTTTTGTTCCCGCCCAAGCCAAAGGACACCCTCATGATTAGTAGAACA |
| | CCGGAGGTTACGTGCGTCGTAGTGGATGTCAGCCACGAGGATCCCGAGGTTAAGTT |
| | TAACTGGTACGTTGATGGGGTTGAGGTCCATAATGCGAAGACTAAGCCGAGAGAGG |
| | AACAGTACAATTCCACGTATAGAGTTGTCTCTGTACTGACTGTGCTGCATCAAGAT |
| | TGGCTTAACGGTAAGGAGTACAAGTGCAAAGTCTCTAATAAGGCTCTTCCTGCACC |
| | CATTGAGAAAACTATAAGCAAAGCAAAAGGTCAACCTCGCGAACCTCAGGTGTACA |
| | CACTGCCACCCTCTAGGGACGAGCTTACCAAAAATCAAGTATCTCTTACCTGCCTT |
| | GTGAAAGGGTTTTATCCCTCAGATATTGCGGTTGAGTGGGAGTCTAACGGACAACC |
| | TGAGAACAACTATAAGACTACTCCCCCGGTGCTTGATTCAGACGGGAGTTTTTTTT |
| | TGTATAGCAAACTTACCGTCGACAAAAGCCGGTGGCAACAGGGCAATGTATTCAGT |
| | TGTTCTGTAATGCATGAAGCTTTGCATAATCATTACACCCAAAAGAGTCTTTCCCT |
| | GTCTCCTGGAAAAGGGGGGTCAGACTCCTGGATGGAGGAGGTGATCAAACTGTGTG |
| | GGAGAGAGCTCGTCCGGGCTCAGATAGCTATATGCGGCAAGTCTACGGGTGGGGGA |
| | GAGGGCGGAGGAGAGGGCGGTGGAGAAGGAGGCGGCCAACTCTACAGCGCTCTGGC |
| | CAATAAATGTTGTCATGTCGGGTGTACTAAGCGCTCACTGGCACGCTTTTGC |
| 387 | ATGGAAACCGACACCCTTTTGTTGTGGGTATTGCTGTTGTGGGTTCCCGGTAGCAC |
| | GGGGGACAAGACGCATACATGCCCGCCATGCCCGGCCCCCGAAGCTGCTGGGGGAC |
| | CATCCGTATTCCTCTTCCCTCCCAAACCAAAAGACACGTTGATGATAAGTAGAACA |
| | CCAGAGGTAACGTGCGTGGTTGTCGATGTTTCCCACGAAGATCCGGAGGTAAAATT |
| | CAATTGGTATGTAGATGGGGTGGAAGTGCACAATGCCAAAACAAAGCCGCGAGAAG |
| | AACAATACAATAGTACTTACCGGGTTGTGAGCGTGCTCACGGTGTTGCACCAAGAC |
| | TGGCTCAACGGCAAGGAATACAAGTGCAAAGTATCTAATAAAGCTCTGCCTGCGCC |
| | GATAGAGAAGACCATCAGTAAGGCCAAAGGGCAGCCCCGAGAGCCGCAAGTTTACA |
| | CTCTTCCTCCGAGCAGAGATGAATTGACCAAGAACCAAGTAAGTTTGACGTGCCTG |
| | GTGAAGGGCTTCTACCCCTCAGACATTGCGGTGGAGTGGGAAAGTAATGGTCAACC |
| | GGAAAACAACTACAAGACCACGCCGCCCGTCCTCGACTCCGATGGGTCTTTCTTTC |
| | TTTATTCAAAGTTGACAGTAGATAAGTCAAGGTGGCAGCAAGGTAACGTGTTTAGT |
| | TGTAGTGTAATGCACGAGGCCCTGCATAATCATTATACCCAAAAGAGTTTGAGCCT |
| | CTCACCAGGAAAAGGCGGATCAGACAGCTGGCAGGAGGAGGTAATTAAATTGTGTG |
| | GACGGGAGTTGGTCAGGGCGCAAATAGCCATCTGCGGTAAGAGCACGGGTGGAGGA |
| | GAGGGTGGAGGGGAAGGTGGGGGAGAAGGCGGCGGGCAGCTCTATTCTGCACTCGC |
| | CAACAAGTGTTGTCACGTCGGATGCACAAAGAGATCTCTTGCTCGATTCTGC |
| 388 | ATGGAGACTGATACTCTTTTGTTGTGGGTACTGCTCCTGTGGGTTCCAGGCTCCAC |
| | AGGAGACAAAACACACACCTGTCCGCCTTGCCCGGCTCCTGAAGCCGCGGGTGGCC |
| | CTAGTGTGTTTTTGTTTCCGCCGAAACCTAAGGATACCCTCATGATAAGCCGGACG |
| | CCCGAGGTTACCTGTGTCGTGGTCGATGTTAGTCATGAGGATCCAGAAGTCAAGTT |
| | TAATTGGTACGTCGACGGCGTTGAAGTCCACAATGCAAAAACTAAACCGCGAGAAG |
| | AACAGTACAACTCCACCTACAGAGTTGTCTCAGTTTTGACAGTTCTCCATCAGGAT |
| | TGGCTCAATGGAAAGGAATATAAGTGCAAGGTCAGCAATAAAGCGCTTGCCGCCCC |
| | TATAGAGAAGACCATTAGCAAGGCGAAAGGACAGCCCCGCGAGCCCCAGGTCTATA |
| | CGCTGCCTCCTAGCAGAGATGAGCTCACGAAAAATCAGGTCAGCTTGACATGCTTG |
| | GTGAAGGGCTTCTACCCCAGTGACATCGCAGTTGAATGGGAGAGCAACGGCCAACC |
| | TGAGAACAACTACAAAACAACGCCCCCGGTTCTTGACAGCGATGGGTCCTTCTTTC |
| | TTTACTCTAAGCTTACAGTTGATAAAAGCAGGTGGCAGCAGGGGAATGTGTTCTCA |
| | TGTTCCGTACTGCATGAGGCTCTGCATTCTCACTACACCCAAAAAAGCCTTAGCCT |
| | GAGCCCCGGTAAGGGAGGTAGTGACTCATGGCAAGAGGAAGTGATTAAGCTCTGCG |
| | GCCGGGAGTTGGTGAGAGCCCAAATCGCCATTTGCGGTAAAAGTACCGGAGGGGGC |
| | GAGGGAGGAGGCGAAGGTGGAGGTGAAGGAGGTGGACAGTTGTACTCAGCTCTTGC |
| | AAATAAATGTTGTCATGTTGGTTGCACGAAAAGATCTCTTGCGAGGTTCTGT |
| 389 | ATGGAGACTGACACTCTTTTGTTGTGGGTGCTTCTTCTGTGGGTACCTGGATCCAC |
| | TGGGGATAAGACGCATACTTGTCCACCGTGCCCCGCACCGGAAGCGGCTGGTGGTC |
| | CATCAGTTTTTCTGTTCCCACCGAAACCTAAGGACACGTTGATGATATCACGGACA |
| | CCAGAGGTTACGTGCGTAGTGGTGGATGTGAGCCACGAGGATCCAGAAGTTAAATT |
| | TAATTGGTACGTAGATGGAGTGGAGGTTCATAATGCGAAGACAAAGCCTCGCGAGG |
| | AACAGTATAATTCCACCTATCGCGTCGTATCTGTGCTTACGGTACTTCACCAAGAC |
| | TGGTTGAACGGTAAGGAATATAAATGCAAGGTTTCCAATAAAGCACTTCCTGCGCC |
| | AATTGAGAAGACAATATCCAAAGCTAAAGGTCAACCCAGGGAACCGCAAGTCTACA |
| | CTCTCCCCCCGTCTCGCGATGAATTGACGAAGAACCAGGTTAGTCTCACCTGCCTG |
| | GTCAAGGGGTTTTACCCCTCTGACATAGCTGTAGAATGGGAGTCTAATGGACAGCC |
| | AGAGAACAATTACAAAACGACCCCCCCGGTCCTCGATTCTGATGGGAGTTTTTTTC |
| | TTTATTCAAAATTGACTGTCGATAAGTCAAGATGGCAACAGGGTAACGTATTTTCT |
| | TGCAGTGTTATGCATGAAGCATTGCACAACCACTATACACAAAAATCATTGAGTTT |
| | GAGTCCCGGTAAAGGGGGAAGCGACTCATGGATGGAAGAAGTAATCAAGCTGTGCG |
| | GGCGAGAGCTTGTGCGAGCTCAGATAGCAATCTGTGGTAAGTCTACAGGTGGAGAG |
| | GGTGGCGGTGAAGAAGGCGGGGGAGAGGGAGGCCAGCTTTATTCTGCCCTGGCTAA |
| | CAAGTGCTGTCACGTTGGATGCACGAAGCGCTCCCTGGCCCGATTCTGC |
| 390 | ATGGAAACCGATACATTGCTTTTGTGGGTCCTCCTTCTTTGGGTTCCTGGCTCTAC |
| | AGGCGATAAGACGCATACTTGTCCCCCATGTCCCGCTCCGGAAGCCGCTGGCGGCC |
| | CCTCCGTTTTTCTGTTCCCGCCGAAACCGAAAGACACCCTGATGATATCACGCACT |
| | CCCGAGGTCACTTGCGTGGTAGTCGATGTTAGTCATGAAGATCCTGAGGTCAAATT |
| | CAATTGGTATGTAGATGGCGTTGAGGTACACAACGCGAAGACAAAACCCCGAGAAG |
| | AACAGTATAACTCAACCTACCGCGTAGTTTCAGTTCTTACCGTACTGCACCAAGAC |
| | TGGTTGAACGGTAAAGAGTACAAATGTAAAGTCAGCAATAAAGCTTTGCCAGCACC |
| | TATCGAAAAAACCATCAGTAAGGCCAAGGGTCAACCCAGGGAGCCGCAAGTGTACA |
| | CTCTTCCCCCTAGCAGGGATGAATTGACCAAGAATCAGGTCTCTTTGACGTGCCTC |
| | GTTAAGGGTTTCTATCCCAGCGATATAGCCGTAGAATGGGAGTCTAACGGTCAGCC |
| | AGAAAATAACTATAAGACAACCCCGCCTGTTTTGGATTCCGACGGCTCTTTTTTTC |
| | TCTACTCTAAGTTGACCGTTGATAAGAGCAGATGGCAGCAGGGAAACGTATTTTCT |
| | TGTTCCGTGATGCACGAAGCCCTGCACAATCACTATACGCAAAAGTCTCTGAGCTT |
| | GAGTCCGGGTAAAGGCGGTTCTGACTCCTGGCAGGAGGAAGTCATAAAACTCTGCG |
| | GAAGAGAGCTCGTAAGGGCGCAAATCGCTATTTGTGGTAAGAGCACCGGTGGGGAA |
| | GGAGGCGGTGAAGAGGGTGGCGGCGAGGGTGGGCAATTGTATTCCGCGCTTGCCAA |
| | TAAATGTTGTCACGTAGGCTGCACAAAGCGAAGTCTCGCTAGGTTCTGC |
| 391 | ATGGAAACCGACACCTTGCTTTTGTGGGTGCTCTTGCTGTGGGTTCCGGGGAGCAC |
| | TGGCGACAAGACCCACACATGTCCCCCGTGTCCGGCACCAGAAGCAGCGGGGGGAC |
| | CGTCAGTATTCTTGTTTCCACCGAAGCCCAAAGACACATTGATGATTTCACGAACT |
| | CCTGAAGTTACCTGTGTGGTTGTAGATGTATCACACGAAGACCCAGAAGTCAAATT |
| | CAATTGGTATGTCGACGGGGTTGAAGTTCACAATGCGAAGACGAAGCCCCGGGAGG |
| | AACAGTACAACAGCACGTACAGGGTTGTGAGCGTTCTTACTGTATTGCACCAGGAT |
| | TGGCTCAACGGCAAGGAGTATAAATGTAAAGTTTCTAATAAGGCTCTTCCTGCCCC |
| | AATTGAAAAGACGATATCTAAAGCGAAGGGCCAACCACGGGAACCTCAGGTGTACA |
| | CACTTCCGCCTAGCAGGGATGAGTTGACCAAGAATCAAGTCTCTTTGACGTGCCTG |
| | GTCAAGGGGTTTTACCCATCAGATATCGCCGTCGAATGGGAGTCAAACGGACAACC |
| | CGAAAATAACTATAAAACTACTCCACCAGTTCTGGATAGCGACGGCTCATTTTTTC |
| | TGTATTCAAAGCTCACTGTAGACAAGTCTAGGTGGCAGCAGGGTAATGTCTTCTCC |
| | TGCTCAGTAATGCATGAGGCTCTTCACAACCACTATACTCAAAAGAGCCTTTCCCT |
| | GTCACCTGGCGGTGGAAGCGACTCATGGATGGAGGAGGTAATAAAGCTCTGCGGAA |
| | GAGAACTGGTACGCGCACAAATCGCAATTTGTGGTAAGAGTACTGGCGGGGAAGGA |
| | GGTGGGGAAGAAGGGGGCGGTGAGGGCGGACAGCTCTATTCTGCACTTGCAAACAA |
| | ATGTTGCCACGTGGGATGTACTAAGCGAAGCCTTGCAAGATTCTGC |
| 392 | ATGGAGACCGACACACTGTTGCTGTGGGTACTCCTCCTGTGGGTGCCAGGAAGCAC |
| | GGGCGATAAAACCCACACATGCCCTCCATGCCCTGCTCCAGAGGCCGCCGGTGGGC |
| | CATCAGTTTTCTTGTTTCCGCCTAAACCAAAGGACACGCTTATGATCTCCAGGACC |
| | CCCGAAGTTACGTGTGTGGTGGTTGATGTTAGTCACGAGGACCCGGAAGTCAAGTT |
| | CAACTGGTACGTTGATGGTGTAGAGGTGCACAATGCAAAGACGAAGCCACGCGAAG |
| | AACAATACAACAGCACATATCGAGTTGTGAGCGTACTCACGGTACTGCATCAGGAC |
| | TGGCTGAACGGTAAAGAATACAAATGTAAAGTCTCCAATAAGGCACTTCCTGCGCC |
| | GATAGAAAAAACGATCAGTAAGGCCAAGGGCCAACCCCGAGAACCACAGGTATATA |
| | CGCTCCCACCGTCACGAGACGAGTTGACAAAAAATCAGGTCTCCCTGACTTGCCTC |
| | GTGAAAGGTTTTTATCCCTCAGATATTGCTGTTGAGTGGGAAAGCAATGGGCAGCC |
| | AGAGAATAATTATAAGACGACTCCTCCGGTTTTGGATTCCGACGGTAGTTTTTTCT |
| | TGTATAGTAAGCTTACTGTAGACAAGTCAAGATGGCAACAAGGTAATGTGTTCTCT |
| | TGCTCAGTTATGCATGAAGCTCTTCATAACCATTACACGCAAAAGAGTCTCAGTCT |
| | GAGCCCCGGTGGCGGTAGCGACAGTTGGCAGGAAGAGGTGATTAAGTTGTGCGGTC |
| | GCGAGCTCGTTCGGGCCCAAATTGCAATCTGCGGAAAATCTACGGGCGGAGAGGGC |
| | GGGGGTGAGGAGGGTGGGGGTGAAGGTGGGCAGCTCTATAGCGCCCTTGCGAATAA |
| | ATGTTGTCACGTCGGATGCACAAAGAGGTCCCTCGCCAGGTTCTGC |
| 393 | ATGGAAACTGACACACTGTTGCTGTGGGTGCTGCTCCTTTGGGTACCCGGATCAAC |
| | CGGGGATAAGACCCACACTTGCCCCCCTTGCCCTGCCCCCGAAGCGGCCGGAGGTC |
| | CTTCAGTATTTTTGTTTCCACCGAAACCCAAAGATACTTTGATGATATCAAGAACT |
| | CCTGAAGTCACCTGCGTGGTAGTTGACGTATCTCATGAGGATCCCGAGGTGAAGTT |
| | CAATTGGTACGTCGATGGCGTCGAGGTTCATAACGCTAAGACTAAGCCGAGGGAAG |
| | AGCAATATAATTCCACTTATAGGGTGGTGTCCGTCTTGACTGTTTTGCACCAGGAT |
| | TGGTTGAACGGGAAAGAGTACAAATGTAAGGTGAGTAATAAAGCTTTGGCTGCTCC |
| | CATCGAAAAGACAATAAGCAAGGCCAAGGGGCAACCTCGGGAGCCGCAGGTGTACA |
| | CCCTTCCTCCCAGTAGAGACGAACTGACAAAAAACCAGGTGTCCCTGACCTGCCTT |
| | GTGAAGGGGTTTTACCCGAGCGACATAGCGGTTGAATGGGAGAGCAACGGGCAACC |
| | CGAGAACAACTACAAAACTACACCGCCTGTCCTGGACTCCGATGGAAGCTTCTTCC |
| | TCTACTCCAAACTGACCGTGGACAAAAGCAGATGGCAACAAGGAAACGTATTCTCA |
| | TGCTCAGTAATGCACGAAGCATTGCACAATCACTACACCCAAAAGTCCCTCTCACT |
| | CTCCCCTGGTAAGGGCGGATCAGACTCATGGCAAGAGGAGGTAATTAAGTTGTGCG |
| | GGAGGGAGCTCGTCCGCGCGCAAATAGCCATTTGTGGCAAGTCCACTGGAGGAGGC |
| | GAGGGTGGAGGAGAGGGTGGTGGGGAGGGCAGGCAACTCTACAGTGCGCTCGCCAA |
| | TAAATGCTGCCATGTTGGGTGCACGAAGCGCAGTCTCGCACAATTCTGC |
| 394 | ATGGAGACCGACACTCTGCTGCTCTGGGTACTCTTGCTGTGGGTGCCTGGGTCTAC |
| | TGGGGATAAGACCCACACGTGTCCTCCATGTCCGGCACCGGAGGCTGCTGGCGGGC |
| | CTTCTGTATTCCTCTTCCCACCCAAGCCAAAAGACACATTGATGATATCAAGGACG |
| | CCGGAAGTCACCTGTGTTGTTGTGGACGTTTCCCATGAAGACCCAGAGGTAAAATT |
| | CAATTGGTATGTGGACGGCGTAGAGGTTCACAACGCCAAAACCAAACCCCGAGAGG |
| | AACAGTATAATAGCACATATCGAGTAGTATCTGTTCTCACAGTGCTCCATCAAGAC |
| | TGGCTTAATGGTAAAGAGTATAAATGCAAAGTTTCCAATAAAGCCCTCGCTGCACC |
| | GATCGAGAAGACAATCAGTAAAGCGAAGGGCCAGCCTCGGGAACCGCAGGTGTATA |
| | CTCTTCCACCCTCAAGAGACGAGCTCACTAAAAACCAAGTTTCATTGACATGCCTC |
| | GTCAAAGGTTTCTACCCATCAGACATCGCGGTCGAATGGGAAAGTAATGGGCAGCC |
| | GGAAAACAACTATAAAACGACGCCGCCCGTCTTGGATTCTGATGGTTCATTTTTTC |
| | TTTACTCTAAATTGACCGTCGATAAAAGTAGGTGGCAACAAGGAAATGTTTTTTCC |
| | TGCTCCGTCCTGCATGAAGCGTTGCACAGTCACTATACCCAGAAGAGTCTTTCTTT |
| | GTCACCCGGAAAAGGCGGTTCAGATTCATGGCAGGAAGAAGTAATTAAACTCTGTG |
| | GCCGCGAGCTTGTTAGGGCGCAGATAGCCATATGTGGTAAAAGCACCGGAGGAGGT |
| | GAAGGCGGAGGCGAAGGAGGTGGGGAAGGAAGACAATTGTATTCTGCACTTGCAAA |
| | TAAATGCTGTCATGTGGGGTGCACGAAACGCAGTCTTGCACAATTTTGT |
| 395 | ATGGAAACCGATACGCTGCTTCTTTGGGTTCTTCTCCTCTGGGTTCCAGGGTCCAC |
| | CGGCGACAAAACCCATACCTGCCCCCCTTGCCCTGCACCAGAAGCGGCGGGAGGAC |
| | CTAGCGTTTTTCTTTTTCCTCCGAAACCGAAAGATACCCTCATGATATCAAGAACA |
| | CCTGAGGTTACTTGCGTTGTCGTGGACGTGAGTCACGAAGACCCCGAGGTGAAGTT |
| | CAACTGGTATGTAGATGGAGTGGAGGTCCATAATGCAAAAACGAAACCGAGAGAAG |
| | AACAATACAACTCTACATATCGAGTCGTGTCAGTACTCACGGTTTTGCATCAAGAT |
| | TGGCTGAACGGTAAGGAGTACAAGTGTAAGGTTAGCAACAAGGCTCTCGCGGCGCC |
| | GATAGAAAAGACTATAAGTAAAGCAAAAGGCCAGCCCAGAGAACCTCAAGTTTACA |
| | CTCTGCCTCCCAGCAGAGATGAACTGACTAAAAATCAGGTTTCATTGACCTGTCTC |
| | GTCAAGGGTTTTTATCCAAGCGACATAGCAGTTGAATGGGAAAGCAACGGTCAACC |
| | AGAAAATAATTACAAAACCACTCCACCAGTCTTGGACTCTGACGGATCCTTCTTTC |
| | TCTATTCAAAATTGACGGTGGATAAATCTAGGTGGCAGCAAGGCAACGTCTTCTCT |
| | TGTAGCGTTATGCATGAGGCGCTGCACAACCACTACACACAAAAGTCTCTTAGTTT |
| | GAGCCCGGGCGGCGGAAGCGACTCTTGGCAAGAGGAAGTGATAAAACTCTGTGGTC |
| | GAGAATTGGTACGCGCGCAGATCGCTATCTGCGGCAAGTCCACAGGGGGAGGGGAA |
| | GGTGGCGGGGAAGGTGGTGGCGAGGGCAGGCAGTTGTATAGTGCACTTGCCAACAA |
| | GTGCTGCCATGTGGGGTGCACCAAGCGCAGTTTGGCACGGTTCTGC |
| 396 | ATGGAAACGGACACCCTTCTGCTCTGGGTACTGCTGCTCTGGGTTCCTGGTTCTAC |
| | CGGTGATAAAACTCACACTTGTCCCCCGTGTCCGGCACCAGAAGCCGCAGGAGGGC |
| | CATCTGTCTTTCTTTTTCCCCCAAAACCCAAGGATACACTGATGATCTCCCGCACT |
| | CCCGAAGTTACTTGTGTCGTAGTAGACGTTTCTCACGAGGACCCAGAGGTGAAATT |
| | CAATTGGTATGTTGACGGAGTAGAGGTGCATAATGCCAAGACAAAGCCCCGAGAGG |
| | AACAATACAATTCAACCTACAGAGTAGTGTCCGTTCTTACGGTTCTCCATCAGGAT |
| | TGGCTCAACGGTAAGGAATATAAGTGCAAGGTAAGCAACAAAGCGCTGGCCGCACC |
| | CATTGAGAAAACCATTTCAAAAGCTAAAGGCCAACCCCGCGAACCACAAGTTTATA |
| | CTCTCCCCCCAAGTCGCGATGAACTTACAAAAAATCAAGTCTCATTGACGTGCTTG |
| | GTCAAAGGCTTCTACCCGAGCGATATCGCTGTTGAATGGGAGTCTAATGGACAACC |
| | GGAAAATAACTATAAAACTACACCCCCAGTCCTCGATTCAGACGGCAGCTTCTTCC |
| | TGTATTCAAAACTGACGGTTGACAAATCACGCTGGCAACAGGGTAACGTTTTTTCC |
| | TGTAGCGTTCTTCATGAAGCCTTGCACAGTCACTACACCCAGAAGTCCCTTAGCTT |
| | GTCACCTGGCGGGGGTTCAGACTCTTGGCAGGAGGAGGTAATCAAACTGTGCGGAA |
| | GAGAACTGGTGAGGGCTCAGATTGCAATTTGTGGGAAGAGCACGGGTGGCGGTGAA |
| | GGAGGTGGCGAGGGCGGAGGAGAGGGGAGGCAACTCTACAGTGCGTTGGCTAATAA |
| | ATGCTGTCACGTCGGCTGTACTAAGAGAAGCCTCGCCAGATTTTGC |
| 397 | ATGGAAACAGATACTTTGTTGCTGTGGGTACTCCTCCTCTGGGTACCTGGGAGCAC |
| | CGGGGACAAGACGCATACTTGCCCTCCGTGCCCTGCACCAGAAGCCGCTGGTGGCC |
| | CATCTGTGTTTTTGTTCCCCCCTAAGCCAAAAGACACATTGATGATTTCACGAACT |
| | CCAGAAGTGACTTGCGTAGTTGTTGACGTATCACACGAAGACCCCGAGGTTAAATT |
| | TAATTGGTATGTGGACGGGGTCGAGGTGCATAACGCCAAAACCAAACCCCGGGAGG |
| | AACAATATAACTCTACGTATCGGGTCGTATCTGTGTTGACCGTCCTTCACCAAGAT |
| | TGGTTGAACGGCAAGGAATATAAGTGTAAAGTGTCTAATAAAGCATTGGCTGCCCC |
| | GATAGAAAAGACGATCTCTAAAGCCAAGGGCCAACCCAGAGAGCCTCAAGTATATA |
| | CTCTCCCACCGAGTCGAGATGAGCTCACTAAGAACCAGGTGTCACTCACGTGTCTG |
| | GTTAAAGGATTTTACCCTAGTGATATAGCCGTCGAGTGGGAATCAAATGGGCAGCC |
| | GGAGAATAACTATAAGACCACGCCTCCAGTTCTCGATTCCGATGGTAGCTTTTTCC |
| | TTTACTCTAAACTTACGGTCGACAAGTCCAGGTGGCAACAGGGCAATGTATTTTCT |
| | TGCTCCGTCATGCACGAGGCTTTGCACAACCATTACACGCAAAAGTCACTGTCCCT |
| | GTCTCCTGGAGGCGGTTCTGACAGTTGGCAGGAGGAGGTAATCAAATTGTGTGGGC |
| | GGGAGTTGGTTAGGGCGCAAATTGCTATTTGCGGCAAAAGTACTGGGGGCGGTGAA |
| | GGCGGAGGCGAGGGAGGAGGAGAAGGTCGACAACTGTATTCTGCCTTGGCGAACAA |
| | ATGCTGTCACGTCGGCTGTACGAAACGGTCTTTGGCCCAGTTTTGT |
| 398 | ATGGAAACTGACACTCTTCTGTTGTGGGTCCTTCTGCTGTGGGTTCCTGGCTCTAC |
| | TGGAGATAAGACACACACTTGTCCGCCATGCCCTGCGCCGGAAGCGGCGGGAGGAC |
| | CGTCCGTTTTCCTGTTCCCTCCCAAACCCAAAGACACGTTGATGATTAGTCGCACG |
| | CCAGAAGTTACGTGCGTTGTCGTAGATGTATCCCACGAAGACCCCGAGGTGAAGTT |
| | CAATTGGTATGTAGATGGGGTGGAGGTCCATAACGCTAAGACCAAACCACGCGAGG |
| | AACAATATAATTCTACGTACCGCGTAGTGAGCGTTCTCACAGTTCTTCACCAGGAT |
| | TGGCTTAACGGCAAGGAGTATAAGTGTAAGGTGTCTAATAAGGCCTTGGCTGCCCC |
| | GATCGAAAAAACGATAAGTAAAGCAAAGGGTCAACCTAGAGAACCCCAAGTGTACA |
| | CTCTCCCGCCATCACGGGATGAATTGACTAAGAACCAAGTGTCACTCACGTGTCTT |
| | GTAAAGGGCTTCTACCCATCCGATATAGCCGTTGAGTGGGAATCCAATGGTCAGCC |
| | AGAGAACAACTATAAGACAACTCCGCCCGTACTTGATAGTGACGGTTCCTTTTTCC |
| | TTTACAGTAAATTGACGGTAGATAAGTCTCGCTGGCAGCAAGGAAACGTCTTTTCT |
| | TGTTCAGTGCTTCATGAGGCGCTTCACTCACACTATACTCAGAAGAGTTTGAGTTT |
| | GTCTCCAGGTGGAGGCAGCGACTCATGGCAAGAGGAAGTAATCAAACTGTGTGGTC |
| | GCGAATTGGTACGAGCACAGATCGCGATCTGCGGGAAATCAACAGGTGGCGGCGAA |
| | GGCGGCGGGGAAGGCGGCGGCGAAGGTAGGCAACTTTACTCAGCCCTTGCGAACAA |
| | ATGTTGCCACGTAGGCTGTACTAAGAGAAGTCTCGCCCAGTTTTGC |
| 399 | ATGGAGACAGATACCCTTCTGTTGTGGGTCCTTCTGCTTTGGGTGCCGGGAAGTAC |
| | AGGCGACAAGACTCATACCTGCCCCCCTTGTCCAGCACCAGAAGCAGCTGGCGGGC |
| | CAAGCGTGTTCCTGTTTCCACCTAAGCCCAAAGATACGTTGATGATCAGCCGCACC |
| | CCGGAAGTAACCTGTGTAGTAGTAGATGTGTCCCACGAAGACCCCGAAGTAAAGTT |
| | TAATTGGTACGTCGATGGTGTCGAAGTACATAACGCTAAAACGAAGCCCCGAGAAG |
| | AGCAGTACAACAGTACTTACAGAGTAGTTTCTGTTCTTACAGTGCTGCATCAGGAT |
| | TGGCTGAACGGGAAGGAGTATAAATGTAAAGTCTCAAACAAGGCACTTGCGGCACC |
| | AATAGAGAAGACAATATCTAAGGCCAAAGGGCAGCCTAGAGAGCCACAAGTATATA |
| | CGCTGCCCCCCAGCAGGGACGAGCTGACAAAGAACCAAGTGTCACTGACCTGCCTT |
| | GTTAAGGGCTTCTATCCGAGTGATATTGCTGTTGAATGGGAAAGTAACGGACAGCC |
| | GGAGAACAACTATAAAACTACTCCACCCGTGTTGGATAGTGACGGTAGCTTTTTTC |
| | TGTACTCCAAGTTGACGGTAGACAAAAGTCGGTGGCAGCAGGGGAACGTATTTTCT |
| | TGTTCTGTCATGCACGAAGCTCTTCACAATCACTATACGCAGAAGTCCCTCTCTCT |
| | CTCTCCTGGGAAGGGTGGTTCCGACAGCTGGCAGGAGGAGGTCATTAAACTGTGTG |
| | GTAGAGAGCTGGTACGGGCTCAAATTGCAATTTGTGGTAAGAGTACTGGCGGTGGC |
| | GAGGAAGGGGGTGGGGAGGAGGGCGGAGGTAGGCAGCTCTACTCTGCTCTCGCCAA |
| | CAAGTGTTGTCACGTCGGGTGTACTAAAAGATCACTTGCCCGCTTTTGT |
| 400 | ATGGAAACCGATACCCTGCTCTTGTGGGTCCTCCTGCTTTGGGTCCCAGGTTCCAC |
| | AGGCGACAAAACACATACATGCCCGCCGTGTCCGGCGCCTGAAGCAGCAGGAGGCC |
| | CCAGTGTATTCCTTTTCCCTCCAAAGCCAAAAGATACGTTGATGATATCTAGGACA |
| | CCTGAGGTTACCTGCGTCGTAGTGGACGTATCCCACGAAGACCCAGAAGTCAAGTT |
| | TAACTGGTATGTGGACGGAGTGGAGGTACACAATGCAAAGACAAAGCCGCGAGAGG |
| | AACAATATAATTCCACCTATAGAGTCGTGTCAGTCCTTACGGTCTTGCACCAGGAC |
| | TGGCTCAATGGTAAGGAGTATAAGTGCAAAGTATCAAACAAAGCTCTCGCAGCGCC |
| | CATCGAAAAGACCATCAGCAAAGCTAAGGGCCAGCCAAGAGAGCCTCAAGTGTACA |
| | CGTTGCCGCCTTCAAGGGACGAGCTCACTAAAAATCAGGTATCACTTACGTGTCTT |
| | GTCAAAGGGTTTTATCCTTCCGACATCGCGGTTGAATGGGAGAGCAATGGACAGCC |
| | GGAGAATAATTATAAAACGACGCCGCCGGTCCTTGACAGCGATGGTTCATTTTTCC |
| | TTTACTCAAAGCTGACGGTTGATAAGTCTAGGTGGCAGCAGGGGAACGTCTTTTCC |
| | TGTAGTGTACTTCATGAGGCGCTCCATTCTCATTACACTCAGAAGTCACTGAGCCT |
| | TTCACCCGGCAAAGGTGGATCAGACTCCTGGCAAGAAGAGGTAATCAAACTCTGTG |
| | GGAGGGAACTCGTTCGAGCCCAGATTGCAATCTGTGGGAAAAGCACAGGCGGAGGG |
| | GAAGAAGGGGGTGGCGAAGAAGGTGGGGGCAGGCAGCTCTATTCAGCTCTTGCCAA |
| | CAAATGCTGTCATGTAGGCTGCACAAAGCGATCACTGGCGAGATTCTGT |
| 401 | ATGGAAACCGACACCCTGCTGCTCTGGGTTCTTCTCCTCTGGGTTCCCGGCTCAAC |
| | CGGAGATAAAACTCATACTTGCCCACCCTGCCCGGCTCCCGAGGCAGCAGGTGGAC |
| | CCTCAGTATTTTTGTTCCCTCCGAAACCTAAAGATACACTTATGATTAGCCGGACC |
| | CCTGAGGTAACGTGTGTGGTGGTTGACGTAAGTCATGAAGATCCAGAAGTAAAGTT |
| | TAACTGGTACGTAGACGGTGTGGAGGTACATAATGCGAAGACAAAACCACGAGAGG |
| | AACAGTATAACTCTACCTACCGCGTAGTAAGCGTACTTACTGTGCTCCACCAAGAC |
| | TGGCTTAACGGGAAAGAGTATAAGTGTAAAGTCAGTAATAAAGCACTGGCCGCCCC |
| | GATCGAAAAAACAATCAGCAAGGCCAAAGGACAACCAAGGGAGCCTCAGGTCTATA |
| | CTCTTCCCCCGAGTAGGGATGAGCTTACCAAGAACCAGGTGTCTCTGACATGCCTT |
| | GTCAAGGGATTTTACCCGAGTGACATAGCCGTAGAATGGGAGTCAAACGGCCAACC |
| | TGAAAACAACTATAAGACCACGCCTCCCGTACTCGACTCAGATGGAAGCTTTTTCC |
| | TCTATAGCAAGCTGACCGTCGACAAAAGTAGGTGGCAACAGGGAAACGTCTTTAGT |
| | TGTTCCGTCATGCACGAAGCTTTGCATAACCATTACACCCAGAAGAGTCTTTCCCT |
| | TTCCCCTGGCAAGGGGGGCTCCGACTCCTGGCAAGAGGAAGTAATCAAACTGTGTG |
| | GGCGCGAGCTTGTCCGCGCGCAAATAGCCATTTGCGGAAAAAGTACTGGAGGAGGA |
| | GAGGAAGGCGGCGGCGAGGAAGGTGGGGGCAGGCAGCTGTACAGTGCCTTGGCTAA |
| | CAAGTGCTGCCATGTCGGCTGTACGAAAAGGTCTCTTGCTCAATTCTGT |
| 402 | ATGGAAACTGATACTCTTCTCCTTTGGGTGCTCCTCCTCTGGGTTCCCGGGTCCAC |
| | AGGCGATAAGACACATACCTGTCCACCCTGCCCAGCACCTGAAGCTGCAGGCGGCC |
| | CCAGCGTATTCCTGTTTCCTCCGAAGCCGAAAGACACACTTATGATTTCCCGGACG |
| | CCTGAGGTAACTTGCGTCGTAGTAGATGTGTCTCACGAAGACCCCGAGGTGAAATT |
| | CAACTGGTACGTTGATGGTGTGGAAGTTCATAATGCGAAAACTAAACCACGAGAGG |
| | AGCAATATAACTCAACTTATAGAGTTGTGAGCGTCTTGACGGTACTGCACCAGGAC |
| | TGGCTGAATGGCAAAGAGTACAAATGCAAAGTCTCAAATAAGGCGTTGGCGGCTCC |
| | CATAGAGAAAACTATCAGCAAAGCCAAGGGTCAACCTCGGGAGCCACAAGTGTATA |
| | CTCTTCCGCCTAGTCGCGACGAGCTCACAAAGAATCAGGTGAGTCTTACTTGTTTG |
| | GTTAAGGGTTTCTACCCCAGTGACATTGCGGTCGAGTGGGAAAGTAACGGACAGCC |
| | TGAAAACAACTATAAAACAACGCCTCCAGTACTCGATTCAGATGGTTCATTCTTTC |
| | TTTATTCCAAACTCACAGTCGACAAGAGTAGATGGCAACAAGGGAACGTGTTTAGC |
| | TGTAGCGTACTCCATGAGGCACTCCACTCTCACTATACCCAAAAGTCTCTCAGCTT |
| | GTCACCCGGAAAAGGCGGTTCTGACAGTTGGCAAGAGGAAGTGATTAAATTGTGTG |
| | GGCGGGAACTTGTGAGGGCTCAAATCGCGATTTGCGGCAAGTCCACTGGTGGCGGC |
| | GAGGAAGGAGGAGGTGAAGAAGGAGGAGGTAGGCAACTGTATTCAGCGTTGGCGAA |
| | TAAATGCTGCCATGTTGGATGTACTAAACGGAGCCTTGCTCAGTTCTGC |
| 403 | ATGGAAACTGACACCTTGTTGCTTTGGGTATTGCTTCTGTGGGTTCCGGGTAGCAC |
| | GGGTGATAAAACGCATACTTGCCCTCCTTGCCCGGCACCTGAAGCTGCCGGAGGTC |
| | CTTCCGTGTTCCTGTTCCCACCTAAGCCAAAAGACACACTTATGATTTCTCGCACA |
| | CCAGAAGTAACGTGCGTCGTAGTTGACGTCTCCCATGAAGACCCGGAGGTAAAATT |
| | TAATTGGTACGTCGACGGGGTAGAAGTTCATAACGCAAAGACTAAACCACGAGAAG |
| | AGCAATACAACTCTACATACAGAGTAGTAAGCGTTCTCACCGTTCTTCATCAAGAT |
| | TGGCTCAACGGAAAGGAGTATAAGTGTAAGGTGTCCAATAAAGCGTTGGCCGCACC |
| | AATCGAAAAGACCATAAGCAAAGCCAAAGGCCAACCCCGCGAACCGCAGGTGTACA |
| | CACTTCCCCCGTCCAGGGATGAATTGACAAAAAACCAAGTTTCCCTCACGTGTCTC |
| | GTCAAGGGATTCTACCCGAGTGATATCGCAGTTGAATGGGAAAGCAATGGTCAGCC |
| | CGAGAATAACTACAAGACTACTCCCCCTGTGTTGGACTCAGACGGCTCATTCTTCC |
| | TCTACAGTAAGTTGACTGTGGACAAAAGTCGGTGGCAGCAAGGCAATGTCTTCAGT |
| | TGTAGTGTAATGCATGAAGCACTCCACAATCATTACACCCAAAAATCCCTGAGCCT |
| | GTCCCCGGGCGGAGGTTCAGATTCATGGCAGGAGGAAGTTATAAAACTGTGCGGGC |
| | GCGAGTTGGTGAGGGCGCAGATCGCAATCTGTGGAAAGAGTACGGGAGGTGGCGAA |
| | GAGGGTGGTGGAGAAGAGGGAGGAGGTCGACAACTGTATTCCGCGCTCGCGAACAA |
| | GTGTTGCCACGTTGGCTGCACCAAACGAAGCCTGGCTCGATTTTGC |
| 404 | ATGGAGACTGACACCCTTCTCCTCTGGGTCCTCTTGCTTTGGGTCCCTGGCTCTAC |
| | TGGTGACAAGACACACACTTGTCCACCTTGCCCGGCTCCCGAGGCGGCAGGAGGAC |
| | CAAGCGTTTTTCTGTTCCCTCCCAAACCAAAGGATACGCTTATGATCTCTCGAACG |
| | CCGGAAGTTACTTGCGTAGTAGTTGATGTCTCCCATGAAGATCCCGAAGTGAAGTT |
| | CAACTGGTATGTAGATGGTGTGGAAGTTCATAACGCGAAAACCAAACCACGCGAAG |
| | AACAGTATAACAGTACTTATCGGGTTGTTTCAGTACTCACGGTGCTCCATCAAGAC |
| | TGGCTTAATGGAAAGGAGTATAAATGTAAGGTAAGTAACAAGGCATTGGCGGCTCC |
| | CATCGAGAAGACAATCTCCAAAGCAAAAGGGCAACCACGGGAGCCTCAGGTGTATA |
| | CGTTGCCGCCCAGCAGAGATGAACTTACTAAGAATCAGGTGAGTCTCACTTGTCTC |
| | GTCAAGGGCTTCTATCCCAGCGATATAGCCGTAGAATGGGAGAGTAACGGTCAGCC |
| | GGAGAACAACTACAAAACAACCCCGCCTGTTTTGGACTCCGATGGGAGTTTTTTTC |
| | TCTACAGCAAACTCACGGTAGACAAAAGCAGGTGGCAGCAGGGCAATGTTTTCAGT |
| | TGCTCTGTTCTCCACGAAGCCCTCCACTCCCACTATACTCAGAAGTCTCTGAGTCT |
| | CTCACCAGGGGGAGGTAGCGATAGCTGGCAGGAGGAAGTGATCAAGTTGTGCGGGC |
| | GCGAACTCGTGCGGGCACAAATTGCTATATGCGGTAAAAGTACGGGAGGTGGAGAG |
| | GAGGGTGGAGGTGAAGAAGGCGGTGGTAGACAATTGTATAGTGCGCTCGCCAACAA |
| | GTGTTGTCATGTCGGGTGTACGAAACGGTCCTTGGCGCGGTTTTGC |
| 405 | ATGGAAACTGACACACTTCTTCTGTGGGTACTCTTGTTGTGGGTTCCGGGCTCAAC |
| | GGGTGACAAGACACATACTTGTCCACCATGTCCCGCCCCAGAAGCTGCGGGAGGAC |
| | CATCAGTTTTTTTGTTCCCCCCGAAACCGAAGGATACCCTCATGATAAGTCGAACG |
| | CCCGAAGTCACTTGCGTGGTGGTTGATGTTAGCCACGAGGACCCAGAAGTGAAGTT |
| | CAACTGGTACGTGGACGGGGTCGAAGTTCATAATGCGAAAACAAAGCCTCGCGAGG |
| | AACAGTACAACTCTACATACAGGGTTGTGTCTGTTTTGACAGTCTTGCACCAAGAT |
| | TGGCTCAACGGGAAGGAATATAAGTGTAAGGTAAGCAATAAAGCACTGGCGGCCCC |
| | GATCGAAAAAACGATATCCAAGGCCAAGGGCCAGCCCCGAGAGCCTCAGGTATATA |
| | CTCTGCCGCCAAGCCGGGATGAACTGACTAAAAACCAGGTCTCTTTGACTTGTCTT |
| | GTCAAGGGATTTTACCCAAGTGACATTGCGGTAGAGTGGGAAAGCAACGGTCAACC |
| | AGAAAACAATTACAAGACGACACCGCCGGTACTCGACTCAGATGGATCCTTTTTCC |
| | TGTATAGCAAGCTGACAGTGGACAAGTCCCGGTGGCAGCAAGGGAACGTATTTTCA |
| | TGCAGCGTGATGCATGAGGCTCTTCACAACCATTACACACAGAAAAGTCTGTCATT |
| | GAGCCCTGGCGGCGGGAGCGATTCTTGGCAAGAAGAAGTTATAAAACTTTGCGGTC |
| | GAGAGCTGGTTCGGGCACAAATTGCTATCTGCGGAAAATCTACAGGAGGAGGCGAG |
| | GAGGGAGGGGGCGAAGAAGGCGGGGGGAGACAGTTGTACAGTGCGCTCGCTAACAA |
| | GTGTTGCCACGTCGGTTGCACAAAGAGATCCCTGGCTCAATTCTGT |
| 406 | ATGGAGACAGATACTCTCTTGCTGTGGGTGCTGCTCTTGTGGGTTCCTGGAAGTAC |
| | CGGTGATAAAACTCACACCTGTCCCCCGTGTCCCGCACCAGAAGCGGCCGGTGGTC |
| | CCTCCGTTTTTCTCTTCCCTCCTAAACCTAAGGACACACTTATGATTAGCAGAACT |
| | CCAGAAGTTACGTGCGTAGTCGTTGACGTTAGTCATGAAGATCCTGAGGTTAAGTT |
| | CAACTGGTACGTAGACGGAGTAGAGGTCCACAACGCCAAGACGAAACCCCGAGAAG |
| | AGCAGTATAATTCTACCTATCGAGTTGTTTCAGTATTGACGGTGCTTCACCAAGAT |
| | TGGCTGAATGGCAAAGAGTATAAGTGCAAGGTAAGCAACAAAGCACTCGCGGCTCC |
| | TATCGAGAAAACTATTTCCAAAGCTAAGGGCCAGCCTCGCGAACCACAAGTCTATA |
| | CCCTGCCACCGAGTCGGGACGAACTCACCAAGAACCAAGTGTCTCTTACTTGCCTC |
| | GTTAAAGGTTTTTATCCCAGCGACATAGCCGTCGAATGGGAGTCCAATGGCCAACC |
| | TGAGAACAACTATAAAACTACCCCTCCTGTACTTGATAGCGACGGAAGTTTTTTCC |
| | TCTATTCAAAACTCACAGTTGATAAGTCTCGATGGCAACAGGGCAACGTCTTCTCT |
| | TGCAGTGTGTTGCATGAAGCTCTGCACTCTCATTACACACAGAAGAGTTTGTCTCT |
| | CAGTCCAGGTGGCGGCTCAGATAGCTGGCAGGAAGAAGTAATCAAGTTGTGCGGCA |
| | GGGAACTGGTAAGGGCACAGATAGCCATTTGTGGAAAATCTACGGGTGGCGGTGAG |
| | GAAGGCGGCGGAGAAGAAGGGGGAGGTCGGCAGCTGTATAGTGCACTCGCAAACAA |
| | GTGCTGCCATGTCGGGTGCACCAAGCGATCCCTTGCCCAGTTTTGC |
| 407 | ATGGAGACGGACACTCTTCTCCTGTGGGTTCTCCTCTTGTGGGTTCCAGGATCTAC |
| | CGGCGATAAGACGCACACATGCCCACCCTGTCCTGCGCCTGAAGCCGCGGGGGGAC |
| | CCAGCGTTTTTCTCTTCCCGCCGAAACCGAAAGACACACTTATGATCAGCCGGACT |
| | CCCGAGGTTACCTGCGTGGTGGTAGATGTATCTCACGAGGATCCCGAGGTCAAATT |
| | CAACTGGTACGTTGATGGGGTTGAAGTTCATAATGCCAAAACGAAGCCAAGAGAAG |
| | AGCAGTATAACTCCACATATAGAGTTGTTTCCGTCTTGACTGTTCTTCACCAAGAT |
| | TGGCTGAATGGGAAGGAGTACAAATGTAAAGTTAGCAACAAGGCACTCGCCGCTCC |
| | CATTGAAAAAACTATAAGCAAAGCTAAGGGCCAACCGCGCGAACCACAGGTCTACA |
| | CGTTGCCGCCCTCTAGGGACGAACTCACGAAGAATCAGGTTTCCCTTACCTGCCTC |
| | GTTAAAGGATTCTACCCCTCTGACATAGCGGTTGAATGGGAGAGCAACGGTCAGCC |
| | TGAGAACAACTACAAAACGACGCCTCCGGTGTTGGATTCCGACGGTAGTTTTTTCC |
| | TCTATAGTAAGCTGACAGTGGATAAATCTCGGTGGCAGCAAGGGAATGTATTCTCC |
| | TGTTCAGTCCTGCATGAAGCCCTCCACTCCCATTATACACAGAAATCTCTTTCTCT |
| | GAGTCCCGGTAAAGGTGGGAGTGACTCTTGGCAGGAAGAGGTAATTAAGTTGTGTG |
| | GAAGGGAGCTGGTAAGAGCACAGATTGCCATCTGTGGCAAATCCACGGGCGGCGAA |
| | GGTGAGGGGGGTGAGGGGGAAGGGGGGTCCAGACAACTGTATTCTGCTCTGGCGAA |
| | TAAGTGTTGCCATGTAGGGTGCACTAAACGGTCCTTGGCGCAGTTCTGT |
| 408 | ATGGAGACTGACACACTGCTCCTCTGGGTCCTTTTGCTCTGGGTTCCGGGGTCCAC |
| | CGGTGATAAAACTCATACGTGCCCACCTTGCCCCGCACCGGAGGCTGCTGGAGGAC |
| | CCTCTGTCTTCCTGTTCCCGCCGAAGCCTAAAGACACATTGATGATCAGTCGAACA |
| | CCGGAAGTCACCTGTGTAGTGGTTGATGTGAGCCATGAGGACCCTGAAGTAAAATT |
| | TAACTGGTATGTTGATGGCGTAGAAGTACACAACGCGAAGACTAAACCAAGGGAAG |
| | AGCAATACAACTCTACCTATAGGGTCGTTAGCGTACTGACTGTGCTTCACCAAGAC |
| | TGGCTTAACGGGAAGGAGTACAAGTGCAAAGTGAGCAATAAGGCCCTCGCCGCGCC |
| | TATCGAGAAAACCATTTCCAAAGCCAAGGGTCAACCAAGGGAGCCTCAGGTTTACA |
| | CCCTGCCCCCTTCAAGGGATGAGTTGACAAAAAACCAGGTAAGTCTGACGTGTCTC |
| | GTTAAGGGATTCTACCCGTCAGATATCGCGGTAGAGTGGGAGAGCAACGGTCAGCC |
| | AGAAAATAATTACAAAACAACACCTCCAGTTTTGGACTCTGATGGGAGTTTTTTTC |
| | TTTATTCTAAGTTGACAGTGGATAAGTCACGCTGGCAACAGGGGAACGTATTTAGC |
| | TGCTCAGTACTTCATGAAGCGTTGCATTCTCACTACACACAGAAGAGCCTCTCCTT |
| | GAGTCCCGGAGGTGGCTCTGATTCTTGGCAGGAGGAGGTAATAAAACTTTGTGGTA |
| | GAGAACTGGTTCGCGCTCAGATAGCTATTTGTGGAAAATCCACTGGCGGTGAAGGT |
| | GAAGGTGGAGAAGGAGAGGGCGGAAGCCGGCAGTTGTACTCTGCCCTGGCTAATAA |
| | GTGCTGTCACGTGGGCTGCACTAAGCGGAGCTTGGCAAGATTTTGC |
| 409 | ATGGAAACCGACACGCTGCTGCTGTGGGTGCTGTTGTTGTGGGTTCCAGGCTCAAC |
| | TGGCGATAAAACTCATACCTGTCCACCTTGTCCTGCGCCTGAGGCAGCTGGAGGGC |
| | CTAGCGTGTTCCTGTTCCCCCCCAAACCCAAAGACACGCTCATGATTAGCCGAACC |
| | CCTGAAGTGACCTGCGTTGTTGTGGACGTAAGCCACGAAGACCCCGAAGTTAAGTT |
| | TAATTGGTACGTCGACGGTGTTGAGGTTCATAACGCGAAGACTAAGCCGAGAGAGG |
| | AGCAATATAACAGCACCTACCGCGTAGTCTCAGTTCTTACCGTGCTCCACCAGGAC |
| | TGGCTTAACGGGAAGGAATACAAATGCAAAGTTTCCAACAAAGCCTTGGCAGCCCC |
| | AATAGAGAAGACAATATCTAAGGCGAAAGGCCAACCGCGGGAACCGCAAGTTTATA |
| | CCCTCCCACCGAGCAGGGATGAGCTGACAAAAAATCAGGTTTCCCTCACTTGTCTG |
| | GTCAAGGGATTTTATCCTTCAGACATAGCCGTTGAATGGGAGAGTAATGGGCAGCC |
| | GGAGAATAATTACAAGACCACCCCCCCGGTGTTGGACAGCGACGGTTCCTTCTTTC |
| | TCTATTCTAAACTTACCGTCGACAAATCACGGTGGCAACAAGGAAATGTATTCTCA |
| | TGCAGTGTATTGCACGAAGCTCTGCACTCTCATTACACCCAAAAATCCCTCTCTCT |
| | CAGCCCTGGCGGTGGATCTGATTCTTGGCAGGAAGAGGTGATTAAACTGTGTGGGC |
| | GAGAGCTTGTCCGAGCTCAGATCGCTATTTGTGGCAAGAGTACCGGAGGCGAGGGT |
| | GAGGGAGGCGAAGGCGAGGGCGGAAGCCGGCAACTCTATAGCGCACTCGCTAATAA |
| | ATGTTGTCATGTCGGCTGCACGAAGCGCTCACTGGCGCAGTTCTGC |
| 500 | ATGGAAACTGATACCCTGTTGTTGTGGGTCCTTTTGCTTTGGGTTCCAGGCAGCAC |
| | CGGAGATAAAACCCATACGTGTCCTCCATGCCCAGCTCCCGAGCTGCTCGGTGGTC |
| | CTTCAGTGTTCCTCTTCCCCCCAAAGCCGAAGGACACGCTCATGATTAGTCGAACG |
| | CCAGAGGTGACATGTGTGGTCGTTGATGTTTCCCATGAGGATCCGGAAGTTAAGTT |
| | CAACTGGTACGTAGATGGCGTGGAGGTTCACAATGCAAAAACCAAGCCCCGCGAGG |
| | AGCAGTATAACTCAACCTACAGAGTAGTATCTGTGCTCACGGTCTTGCATCAGGAT |
| | TGGTTGAACGGGAAGGAATACAAGTGTAAAGTAAGTAATAAGGCACTGCCGGCCCC |
| | CATAGAAAAAACTATCAGCAAAGCTAAAGGTCAGCCGCGGGAGCCACAAGTTTACA |
| | CTCTTCCTCCTAGTAGAGACGAGCTGACGAAGAATCAAGTTTCTTTGACTTGTCTC |
| | GTGAAGGGATTCTACCCAAGCGATATAGCTGTAGAGTGGGAAAGCAACGGACAACC |
| | AGAAAATAACTACAAGACTACACCCCCCGTTCTCGATTCTGATGGCTCATTCTTCT |
| | TGTACTCAAAATTGACAGTTGACAAATCTCGATGGCAGCAGGGTAACGTATTTAGT |
| | TGCTCTGTTATGCACGAAGCGTTGCATAACCACTACACACAGAAGTCATTGTCACT |
| | GAGCCCAGGAAAAGGTGGTGGCGGGTCCGGCGGTGGAGGTAGCGGTGGCGGGGGCT |
| | CCCAGCTTTATAGTGCCCTTGCAAACAAATGTTGCCACGTCGGATGTACGAAGCGC |
| | AGTTTGGCGAGATTCTGTGGAGGGGGCGGATCCGGAGGCGGGGGGTCCGGAGGAGG |
| | AGGTAGCTCATGGATGGAAGAGGTAATAAAACTGTGCGGACGCGAGCTTGTCAGGG |
| | CCCAAATCGCAATTTGTGGCATGAGCACATGGAGT |
| 540 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGAGAAGGGGGCGGAAGGCAGCTTTACTCTGCTCTGGCTAA |
| | CAAGTGTTGTCACGTTGGGTGTACCAAGCGGTCCCTTGCGCAATTCTGC |
| 541 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGAGAAGGGGGCGGAAGGCAGCTTTACTCTGCTCTGGCTAA |
| | CAAGTGTTGTCAAGTTGGGTGTACCAAGCGGTCCCTTGCGCAATTCTGC |
| 542 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGAGAAGGGGGCGGAAGGCAGCTTTACTCTGCTCTGGCTAA |
| | CAAGTGTTGTCACGTTGGGTGTACCAAGCAATCCCTTGCGCAATTCTGC |
| 543 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGAGAAGGGGGCGGAAGGCAGCTTTACTCTGCTCTGGCTAA |
| | CAAGTGTTGTCAAGTTGGGTGTACCAAGCAATCCCTTGCGCAATTCTGC |
| 544 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGATCTGGGGGCGGAAGGCAGCTTTACTCTGCTCTGGCTAA |
| | CAAGTGTTGTCACGTTGGGTGTACCAAGCGGTCCCTTGCGCAATTCTGC |
| 545 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGATCTGGGGGCGGAAGGCAGCTTTACTCTGCTCTGGCTAA |
| | CAAGTGTTGTCAAGTTGGGTGTACCAAGCGGTCCCTTGCGCAATTCTGC |
| 546 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGATCTGGGGGCGGAAGGCAGCTTTACTCTGCTCTGGCTAA |
| | CAAGTGTTGTCACGTTGGGTGTACCAAGCAATCCCTTGCGCAATTCTGC |
| 547 | ATGGAAACGGATACGCTGTTGTTGTGGGTTCTGCTCTTGTGGGTGCCAGGGAGCAC |
| | AGGTGATAAAACCCACACTTGCCCACCTTGCCCTGCGCCGGAAGCCGCCGGAGGAC |
| | CTAGTGTTTTCCTCTTTCCCCCTAAGCCCAAAGACACGTTGATGATCTCTCGGACA |
| | CCGGAAGTAACTTGTGTCGTTGTGGATGTGTCACATGAGGATCCCGAGGTGAAATT |
| | TAATTGGTACGTTGACGGCGTGGAGGTGCATAACGCAAAGACTAAACCACGCGAGG |
| | AGCAGTATAATTCTACATACCGGGTTGTCTCAGTTCTCACAGTTCTTCATCAGGAT |
| | TGGTTGAATGGAAAGGAGTACAAATGCAAAGTGTCCAACAAAGCGCTTGCTGCGCC |
| | GATTGAAAAGACGATTTCAAAGGCAAAAGGGCAGCCCCGCGAACCCCAAGTATATA |
| | CTTTGCCTCCCTCACGCGATGAACTGACTAAGAACCAGGTGAGCCTGACTTGTTTG |
| | GTTAAGGGTTTTTATCCAAGTGACATTGCTGTTGAATGGGAGTCCAATGGCCAGCC |
| | TGAGAATAACTACAAAACGACACCTCCTGTACTTGACAGCGACGGCTCCTTTTTTC |
| | TTTATTCAAAACTCACAGTGGACAAATCCAGGTGGCAGCAGGGTAACGTCTTTTCT |
| | TGCAGCGTGCTCCACGAAGCTTTGCATTCACATTATACGCAAAAATCCTTGTCATT |
| | GTCCCCAGGTAAGGGCGGAAGCGACTCATACCAAGAAGAAGTCATTAAACTTTGTG |
| | GACGGGAGCTGGTTAGGGCACAGATTGCTATTTGTGGTAAGTCTACGGGAGGAGAA |
| | GGCTCTGGAGGCGAAGGATCTGGGGGCGGAAGGCAGCTTTACTCTGCTCTGGCTAA |
| | CAAGTGTTGTCAAGTTGGGTGTACCAAGCAATCCCTTGCGCAATTCTGC |

In some embodiments, any of the nucleotide sequences shown in Table 10 further comprise additional nucleotide sequence on their 5′ and/or 3′ ends. In some embodiments, any of the nucleotide sequences shown in Table 10 further comprise the nucleotide sequence ACGGGACCGATCCAGCCTCCGGACTCTAGAGCCACC (SEQ ID NO: 494) on their 5′ ends and/or any of the nucleotide sequences shown in Table 10 further comprise the nucleotide sequence TGATAAACCGGTTAGTAATGAGTTTGATATCTCGAC (SEQ ID NO: 495) on their 3′ ends.

A variety of host cells and expression vector systems can be utilized to express the fusion proteins described herein. Such expression systems represent vehicles by which the coding sequences of interest can be produced and subsequently purified, but also represent cells which can, when transformed or transfected with the appropriate nucleotide coding sequences, express a fusion protein described herein in situ. These include but are not limited to microorganisms such as bacteria (e.g., E. coli and B. subtilis) transformed with, e.g., recombinant bacteriophage DNA, plasmid DNA or cosmid DNA expression vectors containing fusion protein coding sequences; yeast (e.g., Saccharomyces Pichia) transformed with, e.g., recombinant yeast expression vectors containing fusion protein coding sequences; insect cell systems infected with, e.g., recombinant virus expression vectors (e.g., baculovirus) containing fusion protein coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing fusion protein coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20, and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In certain embodiments, cells for expressing the fusion proteins described herein are human cells, e.g., human cell lines. In certain embodiments, a mammalian expression vector is pOptiVEC™ or pcDNA3.3. In certain embodiments, bacterial cells such as Escherichia coli, or eukaryotic cells (e.g., mammalian cells), are used for the expression of a fusion protein. For example, mammalian cells such as CHO or HEK293 cells, in conjunction with a vector such as the major intermediate early gene promoter element from human cytomegalovirus is an effective expression system for fusion proteins disclosed herein.

In bacterial systems, a number of expression vectors can be advantageously selected depending upon the use intended for the fusion protein being expressed. For example, when a large quantity of a fusion protein is to be produced, vectors which direct the expression of high levels of fusion protein products that are readily purified can be desirable. Such vectors include, but are not limited to, the E. coli expression vector pUR278 (Ruether U & Mueller-Hill B (1983) EMBO J 2:1791-1794), in which the fusion protein coding sequence can be ligated individually into the vector in frame with the lac Z coding region so that a fusion protein is produced; pIN vectors (Inouye S & Inouye M (1985) Nuc Acids Res 13:3101-3109; Van Heeke G & Schuster S M (1989) J Biol Chem 24:5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The fusion protein coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the fusion protein coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the fusion protein molecule in infected hosts (see, e.g., Logan J & Shenk T (1984) PNAS 81 (12): 3655-9, which is herein incorporated by reference in its entirety). Specific initiation signals can also be required for efficient translation of inserted fusion protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al. (1987) Methods Enzymol. 153:516-544, which is herein incorporated by reference in its entirety).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10, and HsS78Bst cells.

For long-term, high-yield production of recombinant proteins, stable expression cells can be generated. For example, cell lines which stably express a fusion protein described herein can be engineered.

In certain embodiments, rather than using expression vectors which contain viral origins of replication, host cells can be transformed with a polynucleotide (e.g., DNA or RNA) controlled by appropriate transcriptional regulatory elements (e.g., promoter, enhancer, sequences, transcription terminators, polyadenylation sites, etc.), and a selectable marker. Following the introduction of polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express a fusion protein described herein or a fragment thereof.

A number of selection systems can be used, including but not limited to the herpes simplex virus thymidine kinase (Wigler M et al. (1977) Cell 11 (1): 223-32), hypoxanthineguanine phosphoribosyltransferase (Szybalska E H & Szybalski W (1962) PNAS 48 (12): 2026-2034) and adenine phosphoribosyltransferase (Lowy I et al. (1980) Cell 22 (3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al. (1980) PNAS 77 (6): 3567-70; O'Hare K et al. (1981) PNAS 78:1527-31); gpt, which confers resistance to mycophenolic acid (Mulligan R C & Berg P (1981) PNAS 78 (4): 2072-6); neo, which confers resistance to the aminoglycoside G-418 (Wu G Y & Wu C H (1991) Biotherapy 3:87-95; Tolstoshev P (1993) Ann Rev Pharmacol Toxicol 32:573-596; Mulligan R C (1993) Science 260:926-932; Morgan R A & Anderson W F (1993) Ann Rev Biochem 62:191-217; Nabel G J & Felgner P L (1993) Trends Biotechnol 11 (5): 211-5); and hygro, which confers resistance to hygromycin (Santerre R F et al. (1984) Gene 30 (1-3): 147-56), all of which are herein incorporated by reference in their entireties. Methods commonly known in the art of recombinant DNA technology can be routinely applied to select the desired recombinant clone and such methods are described, for example, in Ausubel F M et al. (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al. (eds.), Current Protocols in Human Genetics, John Wiley & Sons, NY (1994); Colbère-Garapin F et al. (1981) J Mol Biol 150:1-14, all of which are herein incorporated by reference in their entireties.

### Pharmaceutical Compositions

The present disclosure provides pharmaceutical compositions comprising the fusion proteins or component peptides described herein or the nucleic acid molecules, or the expression vectors that encode them. The pharmaceutical compositions described herein are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also, Powell et al., “Compendium of excipients for parenteral formulations” PDA (1998) J Pharm Sci Technol 52:238-311.

The dose of the fusion proteins or component peptides described herein or the nucleic acid molecules, or the expression vectors that encode them administered to a patient may vary depending upon the age and the size of the patient, target disease, conditions, route of administration, and the like. The preferred dose is typically calculated according to body weight or body surface area. Depending on the severity of the condition, the frequency and the duration of the treatment can be adjusted. Effective dosages and schedules for administering the fusion proteins or component peptides described herein or the nucleic acid molecules, or the expression vectors that encode them may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition disclosed herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Any pharmaceutical composition described herein can be delivered subcutaneously or intravenously with a standard needle and syringe. In addition, with respect to subcutaneous delivery, a pen delivery device readily has applications in delivering a pharmaceutical composition disclosed herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see, Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending, or emulsifying any of the fusion proteins described herein in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid fusion protein contained is generally about 5 to about 500 mg per dosage form in a unit dose; especially in the form of injection, it is preferred that the aforesaid fusion protein is contained in about 5 to about 100 mg and in about 10 to about 250 mg for the other dosage forms.

### Therapeutic Uses

### Monotherapy

The present disclosure provides methods of enhancing a relaxin-2-related activity in a primary cell, comprising contacting the primary cell with a fusion protein or component peptide described herein. In some embodiments, contacting the primary cell with the fusion protein or component peptide results in enhanced relaxin-2 activity in the cell, e.g., as described above. In some embodiments, contacting the primary cell with the fusion protein or component peptide results in activation of the relaxin-2 receptor (RFXP1) on a cell surface. Activation of RXFP1 on the cell surface can lead to cellular responses, including but not limited to, the elevation of cAMP levels, vasodilation, the expression of angiogenic factors, including VEGF, the expression of MMPs, and collagen degradation. In some embodiments, the cell is selected from the group consisting of endothelial cells, vascular smooth muscle cells, other vascular cells, cardiomyocytes, other cardiac cells, and fibroblasts. In some embodiments, the primary cell is within a subject, as described below.

In certain embodiments, the present disclosure provides methods for activating RXFP1 on a cell surface, comprising administering an effective amount of the fusion proteins or component peptides described herein or the nucleic acid molecules, or the expression vectors that encode them to a subject in need thereof, thereby activating RXFP1 on the surface of the cell. Activation of RXFP1 on the cell surface can lead to cellular responses, including but not limited to, the elevation of cAMP levels, vasodilation, the expression of angiogenic factors, including VEGF, the expression of MMPs, and collagen degradation. In some embodiments, the cell is selected from the group consisting of endothelial cells, vascular smooth muscle cells, other vascular cells, cardiomyocytes, other cardiac cells, and fibroblasts.

This disclosure also provides methods for treating various relaxin-2 associated diseases. As used herein, the term “relaxin-2-associated disease,” is a disease or disorder that is caused by, or associated with, relaxin-2 protein production or relaxin-2 protein activity. The term “relaxin-2-associated disease” includes a disease, disorder or condition that would benefit from an increase in relaxin-2 protein activity. As used herein, the term “relaxin-2-associated disorder” has the same meaning as “relaxin-2-associated disease.”

In certain embodiments, the relaxin-2-associated disease or disorder is selected from the group consisting of kidney diseases, fibrotic diseases, and cardiovascular diseases. In certain embodiments, the relaxin-2-associated disease or disorder is pulmonary hypertension.

There are five groups of pulmonary hypertension as defined by the World Health Organization (WHO). Group 1 is pulmonary arterial hypertension (PAH), the diagnosis of which requires right heart catheterization (RHC) to demonstrate a mean pulmonary arterial (PA) pressure (mPAP)≥20 mm Hg at rest and a pulmonary vascular resistance (PVR) of ≥2 Wood units. Additional criteria to meet Group 1 PAH includes: a mean pulmonary capillary wedge pressure (PCWP)≤15 mm Hg, chronic lung diseases (CLDs) and other causes of hypoxemia are mild or absent; venous thromboembolic disease and PA obstructions are absent; and certain miscellaneous disorders are absent, including systemic disorders (e.g., sarcoidosis, chronic renal insufficiency), hematologic disorders (e.g., myeloproliferative diseases and chronic hemolytic anemias), and metabolic disorders (e.g., glycogen storage disease). Group 1 also includes PAH due to an unknown mechanism (idiopathic PAH) and heritable genetic defects (heritable PAH); PAH developed from drugs and toxins; PAH associated with systemic disorders such as connective tissue diseases, human immuno-deficiency virus (HIV) infection, congenital heart disease, and schistosomiasis; PAH with overt features of venous/capillary involvement; and persistent PH of the newborn.

Group 2 is PH due to left heart disease (PH-LHD), which may be diagnosed clinically when there is sufficient LHD on echocardiography (with or without other confirmatory testing) to explain PH. For patients in whom RHC is performed, an mPAP≥20 mmHg, PCWP≥15 mmHg, and a normal or reduced cardiac output is consistent with a hemodynamic diagnosis of LHD-PH. Important adjunct information is the presence of left atrial (LA) enlargement on an echocardiogram and a left heart catheterization (LHC) to confirm an elevated left ventricular end-diastolic pressure. Once PH-LHD is confirmed, patients should be allocated into one of the following categories: PH-LHD due to heart failure with preserved or reduced ejection fraction (group 2.1), heart failure with reduced ejection fraction (group 2.2), valvular heart disease, or congenital or acquired conditions leading to postcapillary PH (group 2.3; e.g., restrictive cardiomyopathy, constrictive pericarditis, LA myxoma, congenital or acquired inflow/outflow tract obstruction, and congenital cardiomyopathies). There are two subgroups of Group 2 PH that patients can be distinguished into: those who have combined pre- and postcapillary PH (CpcPH) and those who have isolated postcapillary hypertension (IpcPH).

Group 3 is PH due to chronic lung disease and/or hypoxemia, which is the diagnosis of PH due to CLD and/or hypoxemia made by demonstration of PH on RHC or echocardiogram and evidence of moderate to severe lung dysfunction and/or hypoxemia. Patients are allocated into PH due to obstructive lung disease (group 3.1), restrictive lung disease (group 3.2), mixed obstructive and restrictive lung disease (group 3.3), PH associated with hypoventilation (group 3.4) hypoxia without lung disease (group 3.5), or PH due to developmental disorders (group 3.6). In some cases, Group 3 PH may be due to COPD, interstitial lung disease, or obstructive sleep apnea.

Group 4 is PH due to pulmonary artery obstructions and includes mostly patients with chronic thromboembolic PH (CTEPH; group 4.1) as well as PH due to PA obstructions (group 4.2, e.g., benign or malignant tumors, arteritis in the absence of CTD, congenital PA stenosis, parasites).

Group 5 is PH due to multifactorial mechanisms and include patients with PH who do not clearly fit into Group 1 through 4. Group 5 PH can be further classified into those that have: hematologic disorders such as chronic hemolytic anemia (e.g., sickle cell disease, beta thalassemia, or spherocytosis) and myeloproliferative disorders; systemic or metabolic disorders including sarcoidosis, pulmonary Langerhans histiocytosis X, and neurofibromatosis; metabolic disorders including Gaucher disease and glycogen storage disease; chronic renal failure and PH associated with hemodialysis; pulmonary tumor thrombotic microangiopathy; and fibrosing mediastinitis.

In certain embodiments, the relaxin-2-associated disease or disorder is pulmonary hypertension, including, any of the WHO defined Group 1, Group 2, Group 3, Group 4, and Group 5 PH.

In certain embodiments, the relaxin-2-associated disease or disorder is pulmonary hypertension, including, but not limited to, pulmonary arterial hypertension (PAH), pulmonary hypertension due to left heart disease (PH-LHD), combined precapillary and postcapillary pulmonary hypertension (CpcPH), and isolated postcapillary pulmonary hypertension (IpcPH). In certain embodiments, the relaxin-2-associated disease or disorder is pulmonary arterial hypertension (PAH). In certain embodiments, the relaxin-2-associated disease or disorder is pulmonary hypertension due to left heart disease (PH-LHD). In certain embodiments, the relaxin-2-associated disease or disorder is combined precapillary and postcapillary pulmonary hypertension (CpcPH). In certain embodiments, the relaxin-2-associated disease or disorder is isolated postcapillary pulmonary hypertension (IpcPH).

In certain embodiments, the relaxin-2-associated disease or disorder is a Group 2 pulmonary hypertension. In certain embodiments, the relaxin-2-associated disease or disorder is isolated postcapillary pulmonary hypertension (IpcPH). IpcPH includes characteristics such as right ventricular dysfunction; thickening and stiffening of left ventricle (LHD); and compromised kidney function. In certain embodiments, the relaxin-2-associated disease or disorder is selected from the group consisting of right ventricular dysfunction; thickening and stiffening of left ventricle (LHD); and compromised kidney function. In certain embodiments, the relaxin-2-associated disease or disorder is combined pre- and postcapillary pulmonary hypertension (CpcPH). CpcPH includes characteristics such as pulmonary artery narrowing, thickening, stiffening, and/or fibrotic remodeling; right ventricular dysfunction; thickening and stiffening of left ventricle (LHD); and compromised kidney function. In certain embodiments, the relaxin-2-associated disease or disorder is selected from the group consisting of pulmonary artery narrowing, thickening, stiffening, and/or fibrotic remodeling; right ventricular dysfunction; thickening and stiffening of left ventricle (LHD); and compromised kidney function.

In certain embodiments, the relaxin-2-associated disease or disorder is heart failure, including, but not limited to, heart failure with preserved ejection fraction (HFpEF), and heart failure with reduced ejection fraction (HFrEF). In certain embodiments, the relaxin-2-associated disease or disorder is heart failure with preserved ejection fraction (HFpEF). In certain embodiments, the relaxin-2-associated disease or disorder is heart failure with reduced ejection fraction (HFrEF).

In certain embodiments, the relaxin-2-associated disease or disorder is heart disease, including, but not limited to, valvular heart disease.

In certain embodiments, the relaxin-2-associated disease or disorder is Group 2 PH (CpcPH or IpcPH) with heart failure with preserved ejection fraction (HFpEF). In certain embodiments, the relaxin-2-associated disease or disorder is CpcPH with HFpEF. In certain embodiments, the relaxin-2-associated disease or disorder is IpcPH with HFpEF. In certain embodiments, the HFpEF is defined as signs and symptoms of New York Heart Association (NYHA) class II-III heart failure and LVEF≥50% and at least one of (i) Heart Failure Association-Pre-test assessment, Echocardiography and natriuretic peptide score, Functional testing in cases of uncertainty, Final etiology (HFA-PEFF) score ≥5 points; and/or (ii) HFA-PEFF score 2-4 points and abnormal diastolic stress testing or invasive hemodynamic measurements. In certain embodiments, the relaxin-2-associated disease or disorder is CpcPH with NYHA class II-III heart failure and LVEF≥50% and at least one of (i) HFA-PEFF score ≥5 points; and/or (ii) HFA-PEFF score 2-4 points and abnormal diastolic stress testing or invasive hemodynamic measurements. In certain embodiments, the relaxin-2-associated disease or disorder is IpcPH with NYHA class II-III heart failure and LVEF ≥50% and at least one of (i) HFA-PEFF score >5 points; and/or (ii) HFA-PEFF score 2-4 points and abnormal diastolic stress testing or invasive hemodynamic measurements.

In certain embodiments, the relaxin-2-associated disease or disorder is Group 2 PH (CpcPH or IpcPH) with heart failure with mid-range ejection fraction (HFmrEF). In certain embodiments, the relaxin-2-associated disease or disorder is CpcPH with HFmrEF. In certain embodiments, the relaxin-2-associated disease or disorder is IpcPH with HFmrEF. In certain embodiments, HFmrEF is defined as signs and symptoms of New York Heart Association (NYHA) class II-III heart failure and LVEF 40% to 49%. In certain embodiments, the relaxin-2-associated disease or disorder is CpcPH with NYHA class II-III heart failure and LVEF 40% to 49%. In certain embodiments, the relaxin-2-associated disease or disorder is IpcPH with NYHA class II-III heart failure and LVEF 40% to 49%.

In certain embodiments, the relaxin-2-associated disease or disorder is Group 2 PH (CpcPH or IpcPH) with heart failure with reduced ejection fraction (HFrEF). In certain embodiments, the relaxin-2-associated disease or disorder is CpcPH with HFrEF. In certain embodiments, the relaxin-2-associated disease or disorder is IpcPH with HFrEF.

In certain embodiments, CpcPH is based on right heart catheterization (RHC) performed showing pulmonary vascular resistance (PVR)≥3 Wood units, mPAP of >20 mm Hg, PCWP>15 mm Hg or PCWP>12 mm Hg and ≤14 mm Hg with evidence on left atrial volume index (LAVI) on echocardiography of ≥34 mL/m². In certain embodiments, IpcPH is based on RHC performed showing PVR<3 Wood units, mPAP of >20 mm Hg, PCWP>15 mm Hg or PCWP>12 mm Hg and ≤14 mm Hg with evidence on left atrial volume index (LAVI) on echocardiography of ≥34 mL/m².

In certain embodiments, the relaxin-2-associated disease or disorder is a cardiovascular disease associated with pregnancy, including, but not limited to, preeclampsia, post-partum hypertension, post-partum cardiomyopathy, pregnancy induced heart failure, and maternal hypertension complicating the puerperium.

In certain embodiments, the relaxin-2-associated disease or disorder is kidney disease. In certain embodiments, the relaxin-2-associated disease or disorder is chronic kidney disease. In certain embodiments, the relaxin-2-associated disease or disorder is hypertensive kidney disease.

In certain embodiments, the relaxin-2-associated disease or disorder is joint disease. In certain embodiments, the relaxin-2-associated disease or disorder is frozen shoulder (also known as adhesive capsulitis).

Administration of the compositions according to the methods described herein may result in a reduction of the severity, signs, symptoms, or markers of a relaxin-2-associated disease or disorder in a patient with a relaxin-2-associated disease or disorder. By “reduction” in this context is meant a statistically significant decrease in such level. The reduction (absolute reduction or reduction of the difference between the elevated level in the subject and a normal level) can be, for example, at least about 20%, 25%, 30%, 35%, 40%, 45%, 50%, 55%, 60%, 65%, 70%, 75%, 80%, 85%, 90%, or 95%, or to below the level of detection of the assay used.

In certain embodiments, administration of the compositions according to the methods described herein results in pulmonary vasodilation in the patient. In certain embodiments, administration of the compositions according to the methods described herein results in an anti-inflammatory effect in the patient. In certain embodiments, administration of the compositions according to the methods described herein results in an anti-fibrotic effect in the patient. In certain embodiments, administration of the compositions according to the methods described herein results in right ventricular remodeling in the patient. In certain embodiments, administration of the compositions according to the methods described herein results in peripheral vasodilation in the patient. In certain embodiments, administration of the compositions according to the methods described herein results in cardiac relaxation in the patient. In certain embodiments, administration of the compositions according to the methods described herein results in left ventricular remodeling in the patient. In certain embodiments, administration of the compositions according to the methods described herein results in improvement in kidney function in the patient.

In certain embodiments, administration of the compositions according to the methods described herein results in increased renal plasma flow. In certain embodiments, administration of the compositions according to the methods described herein results in increased renal plasma flow that persists after 1 week, 2 weeks, 3 weeks, 4 weeks, 5 weeks, or 1 month, after a single administration. In certain embodiments, the increase in the renal plasma flow in the subject is maintained by at least about 30%, at least about 40%, at least about 50%, at least about 60%, at least about 70%, at least about 75%, at least about 80%, at least about 85%, at least about 90%, or at least about 95% 1 week, 2 weeks, 3 weeks, 4 weeks, 5 weeks, or 1 month after a single administration. In certain embodiments, administration of the compositions according to the methods described herein results in increased renal plasma flow that persists after 1 week, 2 weeks, 3 weeks, 4 weeks, 5 weeks, or 1 month, after a single administration to a human. In certain embodiments, the increase in the renal plasma flow in the subject is maintained by at least about 30%, at least about 40%, at least about 50%, at least about 60%, at least about 70%, at least about 75%, at least about 80%, at least about 85%, at least about 90%, or at least about 95% 1 week, 2 weeks, 3 weeks, 4 weeks, 5 weeks, or 1 month after a single administration to a human.

### Combination Therapies and Formulations

The present disclosure also provides compositions and therapeutic formulations comprising the fusion proteins or component peptides described herein or the nucleic acid molecules, or the expression vectors that encode them in combination with one or more additional therapeutically active components (i.e., therapeutic agents), and methods of treatment comprising administering such combinations to subjects in need thereof.

Exemplary additional therapeutic agents include any therapeutic agents that may be used for the treatment of any relaxin-2-related disorders described herein. Exemplary additional therapeutic agents that may be combined with or administered in combination with the fusion proteins or component peptides described herein or the nucleic acid molecules, or the expression vectors that encode them include, but are not limited to any one or more of: angiotensin II receptor blockers, e.g., azilsartan, candesartan, eprosartan, losartan; ACE inhibitors, e.g., lisinopril, benazepril, captopril, enalapril, moexipril, perindopril, quinapril, trandolapril; calcium channel blockers, e.g., amlodipine, amlodipine and benazepril, amlodipine and valsartan, sacubitril and valsartan, diltiazem, felodipine, isradipine, nicardipine, nimodipine, nisoldipine, verapamil; diuretics, e.g., chlorthalidone, hydrochlorothiazide, metolazone, indapamide, torsemide, furosemide, bumetanide, amiloride, triamterene, spironolactone, eplerenone; aldosterone antagonists, e.g., spironolactone, eplerenone; digoxin, e.g., lanoxin; beta blockers, e.g., carvedilol, metoprolol, bisoprolol; activin signaling inhibitors, e.g., sotatercept; sodium/glucose cotransporter 2 (SGLT2) inhibitors, e.g., empagliflozin, dapagliflozin, bexagliflozin, canagliflozin, ertugliflozin, ipragliflozin, luseogliflozin, remogliflozin etabonate sergliflozin etabonate, sotagliflozin, tofogliflozin, henagliflozin, janagliflozin, mizagliflozin, velagliflozin proline hydrate, enavogliflozin; and glucagon-like peptide-1 (GLP-1) receptor agonists, e.g., exenatide, liraglutide, albiglutide, dulaglutide, lixisenatide, semaglutide, and tirzepatide.

In some embodiments, the additional therapeutic agents that may be combined with or administered in combination with the fusion proteins or component peptides described herein or the nucleic acid molecules, or the expression vectors that encode them include, but are not limited to any one or more of: sotatercept, empagliflozin, dapagliflozin, sacubitril, valsartan, semaglutide, dulaglutide, and tirzepatide.

In some embodiments, the additional therapeutic agents are drugs effective in treating fibrosis, including but not limited to, small molecule drugs and antibodies. Exemplary anti-fibrosis drugs include, but are not limited to, TGF-β inhibitors, e.g., small molecules such as hydronidone, distiertide, or antibodies such as fresolimumab, PDGF or VEGF antagonist, e.g., small molecules such as imatinib, nilotinib, or any drugs that target extracellular factors that are involved in the pathogenesis of fibrosis. The description of exemplary drugs for fibrosis can be found, e.g., Li et al., “Drugs and Targets in Fibrosis, Frontiers in Pharm.” 8: Article 855 (2007), incorporated herein by reference.

The additional therapeutically active component(s) may be administered just prior to, concurrent with, or shortly after the administration of the fusion proteins or component peptides described herein or the nucleic acid molecules, or the expression vectors that encode them.

The present disclosure provides pharmaceutical compositions in which the fusion proteins or component peptides described herein or the nucleic acid molecules, or the expression vectors that encode them is co-formulated with one or more of the additional therapeutically active component(s) as described elsewhere herein.

### Administration Regimens

In some embodiments, multiple doses of the fusion proteins or component peptides described herein or the nucleic acid molecules, or the expression vectors that encode them may be administered to a subject over a defined time course. The methods according to this aspect of the disclosure comprise sequentially administering to a subject multiple doses of the fusion proteins or component peptides described herein or the nucleic acid molecules, or the expression vectors that encode them. As used herein, “sequentially administering” means that each dose of the fusion proteins or component peptides described herein or the nucleic acid molecules, or the expression vectors that encode them is administered to the subject at a different point in time, e.g., on different days separated by a predetermined interval (e.g., hours, days, weeks, or months). The present disclosure provides methods which comprise sequentially administering to the patient a single initial dose of a fusion proteins or component peptides described herein or the nucleic acid molecules, or the expression vectors that encode them, followed by one or more secondary doses of the fusion proteins or component peptides described herein or the nucleic acid molecules, or the expression vectors that encode them, and optionally followed by one or more tertiary doses of the fusion proteins or component peptides described herein or the nucleic acid molecules, or the expression vectors that encode them.

The terms “initial dose,” “secondary doses,” and “tertiary doses,” refer to the temporal sequence of administration of the fusion proteins or component peptides described herein or the nucleic acid molecules, or the expression vectors that encode them. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the fusion proteins or component peptides described herein or the nucleic acid molecules, or the expression vectors that encode them, but generally may differ from one another in terms of frequency of administration. In certain embodiments, however, the amounts of fusion proteins or component peptides described herein or the nucleic acid molecules, or the expression vectors that encode them contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of fusion proteins or component peptides described herein or the nucleic acid molecules, or the expression vectors that encode them, which is administered to a patient prior to the administration of the very next dose in the sequence with no intervening doses.

In one embodiment, each secondary and/or tertiary dose is administered 4 weeks after the immediately preceding dose. In one embodiment, a dose of the fusion protein or component peptide described herein, or the nucleic acid molecules, or the expression vectors that encode them, is administered to a patient once every 4 weeks (Q4W).

The methods according to this aspect of the disclosure may comprise administering to a patient any number of secondary and/or tertiary doses of fusion proteins or component peptides described herein or the nucleic acid molecules, or the expression vectors that encode them. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In one embodiment, one or more of the fusion proteins or component peptides described herein or the nucleic acid molecules, or the expression vectors that encode them are administered to a subject as a weight-based dose. A “weight-based dose” (e.g., a dose in mg/kg) is a dose of the protein or peptides that will change depending on the subject's weight.

In another embodiment, one or more of the fusion proteins or component peptides described herein or the nucleic acid molecules, or the expression vectors that encode them, is administered to a subject as a fixed dose. A “fixed dose” (e.g., a dose in mg) means that one dose of the fusion proteins or component peptides described herein or the nucleic acid molecules, or the expression vectors that encode them is used for all subjects regardless of any specific subject-related factors, such as weight. In one particular embodiment, a fixed dose of fusion proteins or component peptides described herein or the nucleic acid molecules, or the expression vectors that encode them is based on a predetermined weight or age.

In general, a suitable dose of the fusion proteins or component peptides described herein or the nucleic acid molecules, or the expression vectors that encode them can be in the range of about 0.001 to about 200.0 milligram per kilogram body weight of the recipient, generally in the range of about 1 to 50 mg per kilogram body weight. In certain embodiments, the fusion proteins or component peptides described herein or the nucleic acid molecules, or the expression vectors that encode them are administered in the range of about 0.001 mg/kg to about 200 mg/kg. In certain embodiments, the fusion proteins or component peptides described herein or the nucleic acid molecules, or the expression vectors that encode them are administered in the range of 0.001 mg/kg to 200 mg/kg. In certain embodiments, the fusion proteins or component peptides described herein or the nucleic acid molecules, or the expression vectors that encode them are administered in the range of about 0.01 mg/kg to about 100 mg/kg. In certain embodiments, the fusion proteins or component peptides described herein or the nucleic acid molecules, or the expression vectors that encode them are administered in the range of 0.01 mg/kg to 100 mg/kg. In certain embodiments, the fusion proteins or component peptides described herein or the nucleic acid molecules, or the expression vectors that encode them are administered in the range of about 0.1 mg/kg to about 20 mg/kg. In certain embodiments, the fusion proteins or component peptides described herein or the nucleic acid molecules, or the expression vectors that encode them are administered in the range of 0.1 mg/kg to 20 mg/kg. In certain embodiments, the fusion proteins or component peptides described herein or the nucleic acid molecules, or the expression vectors that encode them are administered in the range of about 1 mg/kg to about 50 mg/kg. In certain embodiments, the fusion proteins or component peptides described herein or the nucleic acid molecules, or the expression vectors that encode them are administered in the range of 1 mg/kg to 50 mg/kg. For example, the fusion proteins or component peptides described herein or the nucleic acid molecules, or the expression vectors that encode them can be administered at about 0.1 mg/kg, about 0.2 mg/kg, about 0.3 mg/kg, about 0.5 mg/kg, about 1 mg/kg, about 1.5 mg/kg, about 2 mg/kg, about 3 mg/kg, about 5 mg/kg, about 10 mg/kg, about 15 mg/kg, about 20 mg/kg, about 25 mg/kg, about 30 mg/kg, about 40 mg/kg, about 50 mg/kg per single dose. In certain embodiments, the fusion proteins or component peptides described herein or the nucleic acid molecules, or the expression vectors that encode them can be administered at 0.1 mg/kg, 0.2 mg/kg, 0.3 mg/kg, 0.5 mg/kg, 1 mg/kg, 1.5 mg/kg, 2 mg/kg, 3 mg/kg, 5 mg/kg, 10 mg/kg, 15 mg/kg, 20 mg/kg, 25 mg/kg, 30 mg/kg, 40 mg/kg, 50 mg/kg per single dose. Values and ranges intermediate to the recited values are also intended to be part of this disclosure.

In certain embodiments, the fusion proteins or component peptides described herein or the nucleic acid molecules, or the expression vectors that encode them are administered at about 0.3 mg/kg. In certain embodiments, the fusion proteins or component peptides described herein or the nucleic acid molecules, or the expression vectors that encode them are administered at 0.3 mg/kg. In certain embodiments, the fusion proteins or component peptides described herein or the nucleic acid molecules, or the expression vectors that encode them are administered at about 1 mg/kg. In certain embodiments, the fusion proteins or component peptides described herein or the nucleic acid molecules, or the expression vectors that encode them are administered at 1 mg/kg. In certain embodiments, the fusion proteins or component peptides described herein or the nucleic acid molecules, or the expression vectors that encode them are administered at about 3 mg/kg. In certain embodiments, the fusion proteins or component peptides described herein or the nucleic acid molecules, or the expression vectors that encode them are administered at 3 mg/kg. In certain embodiments, the fusion proteins or component peptides described herein or the nucleic acid molecules, or the expression vectors that encode them are administered at about 10 mg/kg. In certain embodiments, the fusion proteins or component peptides described herein or the nucleic acid molecules, or the expression vectors that encode them are administered at 10 mg/kg.

In some embodiments, one or more of the fusion proteins or component peptides described herein or the nucleic acid molecules, or the expression vectors that encode them is administered as a fixed dose of between about 10 mg to about 2500 mg. In some embodiments, one or more of the fusion proteins or component peptides described herein or the nucleic acid molecules, or the expression vectors that encode them is administered as a fixed dose of between 10 mg to 2500 mg. In some embodiments, one or more of the fusion proteins or component peptides described herein or the nucleic acid molecules, or the expression vectors that encode them is administered as a fixed dose of between about 100 mg to about 1500 mg. In some embodiments, one or more of the fusion proteins or component peptides described herein or the nucleic acid molecules, or the expression vectors that encode them is administered as a fixed dose of between 100 mg to 1500 mg. In some embodiments, the fusion proteins or component peptides described herein or the nucleic acid molecules, or the expression vectors that encode them are administered as a fixed dose of about 10 mg, about 15 mg, about 20 mg, 25 mg, about 30 mg, about 50 mg, about 75 mg, about 100 mg, about 125 mg, about 150 mg, about 175 mg, 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1500 mg, about 2000 mg, or about 2500 mg. In some embodiments, the fusion proteins or component peptides described herein or the nucleic acid molecules, or the expression vectors that encode them are administered as a fixed dose of 10 mg, 15 mg, 20 mg, 25 mg, 30 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1500 mg, 2000 mg, or 2500 mg. Values and ranges intermediate to the recited values are also intended to be part of this disclosure.

In some embodiments, the fusion proteins or component peptides described herein or the nucleic acid molecules, or the expression vectors that encode them are administered as a fixed dose of about 150 mg. In some embodiments, the fusion proteins or component peptides described herein or the nucleic acid molecules, or the expression vectors that encode them are administered as a fixed dose of 150 mg. In some embodiments, the fusion proteins or component peptides described herein or the nucleic acid molecules, or the expression vectors that encode them are administered as a fixed dose of at least 150 mg. In some embodiments, the fusion proteins or component peptides described herein or the nucleic acid molecules, or the expression vectors that encode them are administered as a fixed dose of about 300 mg. In some embodiments, the fusion proteins or component peptides described herein or the nucleic acid molecules, or the expression vectors that encode them are administered as a fixed dose of 300 mg. In some embodiments, the fusion proteins or component peptides described herein or the nucleic acid molecules, or the expression vectors that encode them are administered as a fixed dose of about 600 mg. In some embodiments, the fusion proteins or component peptides described herein or the nucleic acid molecules, or the expression vectors that encode them are administered as a fixed dose of 600 mg.

In some embodiments, the fusion proteins or component peptides described herein or the nucleic acid molecules, or the expression vectors that encode them are administered by intravenous administration. In some embodiments, the fusion proteins or component peptides described herein or the nucleic acid molecules, or the expression vectors that encode them are administered by subcutaneous administration.

In some embodiments, the fusion proteins or component peptides described herein or the nucleic acid molecules, or the expression vectors that encode them are administered by intravenous infusion. In some embodiments, the fusion proteins or component peptides described herein or the nucleic acid molecules, or the expression vectors that encode them are administered by intravenous infusion over a duration of 1 minute, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 minutes, 2 hours, 3 hours, 4 hours, or more. In some embodiments, the fusion proteins or component peptides described herein or the nucleic acid molecules, or the expression vectors that encode them are administered by intravenous infusion over 30 minutes. In some embodiments, the fusion proteins or component peptides described herein or the nucleic acid molecules, or the expression vectors that encode them are administered by intravenous infusion over 60 minutes. In some embodiments, the fusion proteins or component peptides described herein or the nucleic acid molecules, or the expression vectors that encode them are administered by intravenous infusion over 30 to 60 minutes.

In some embodiments, the fusion proteins or component peptides described herein or the nucleic acid molecules, or the expression vectors that encode them are administered intravenously at a dose of about 0.3 mg/kg once every 4 weeks. In some embodiments, the fusion proteins or component peptides described herein or the nucleic acid molecules, or the expression vectors that encode them are administered intravenously at a dose of 0.3 mg/kg once every 4 weeks. In certain embodiments, the fusion proteins or component peptides described herein or the nucleic acid molecules, or the expression vectors that encode them are administered at about 0.3 mg/kg once every 4 weeks. In certain embodiments, the fusion proteins or component peptides described herein or the nucleic acid molecules, or the expression vectors that encode them are administered at 0.3 mg/kg once every 4 weeks. In certain embodiments, the fusion proteins or component peptides described herein or the nucleic acid molecules, or the expression vectors that encode them are administered at about 1 mg/kg once every 4 weeks. In certain embodiments, the fusion proteins or component peptides described herein or the nucleic acid molecules, or the expression vectors that encode them are administered at 1 mg/kg once every 4 weeks. In certain embodiments, the fusion proteins or component peptides described herein or the nucleic acid molecules, or the expression vectors that encode them are administered at about 3 mg/kg once every 4 weeks. In certain embodiments, the fusion proteins or component peptides described herein or the nucleic acid molecules, or the expression vectors that encode them are administered at 3 mg/kg once every 4 weeks. In certain embodiments, the fusion proteins or component peptides described herein or the nucleic acid molecules, or the expression vectors that encode them are administered at about 10 mg/kg once every 4 weeks. In certain embodiments, the fusion proteins or component peptides described herein or the nucleic acid molecules, or the expression vectors that encode them are administered at 10 mg/kg once every 4 weeks.

In some embodiments, the fusion proteins or component peptides described herein or the nucleic acid molecules, or the expression vectors that encode them are administered at a dose of about 150 mg once every 4 weeks. In some embodiments, the fusion proteins or component peptides described herein or the nucleic acid molecules, or the expression vectors that encode them are administered at a dose of 150 mg once every 4 weeks. In some embodiments, the fusion proteins or component peptides described herein or the nucleic acid molecules, or the expression vectors that encode them are administered at a dose of at least 150 mg once every 4 weeks. In some embodiments, the fusion proteins or component peptides described herein or the nucleic acid molecules, or the expression vectors that encode them are administered as a fixed dose of about 150 mg once every 4 weeks. In some embodiments, the fusion proteins or component peptides described herein or the nucleic acid molecules, or the expression vectors that encode them are administered as a fixed dose of 150 mg once every 4 weeks. In some embodiments, the fusion proteins or component peptides described herein or the nucleic acid molecules, or the expression vectors that encode them are administered as a fixed dose of at least 150 mg once every 4 weeks. In some embodiments, the fusion proteins or component peptides described herein or the nucleic acid molecules, or the expression vectors that encode them are administered as a fixed dose of about 300 mg once every 4 weeks. In some embodiments, the fusion proteins or component peptides described herein or the nucleic acid molecules, or the expression vectors that encode them are administered as a fixed dose of 300 mg once every 4 weeks. In some embodiments, the fusion proteins or component peptides described herein or the nucleic acid molecules, or the expression vectors that encode them are administered as a fixed dose of about 600 mg once every 4 weeks. In some embodiments, the fusion proteins or component peptides described herein or the nucleic acid molecules, or the expression vectors that encode them are administered as a fixed dose of 600 mg once every 4 weeks.

In some embodiments, the fusion proteins or component peptides described herein or the nucleic acid molecules, or the expression vectors that encode them are administered subcutaneously at a dose of about 150 mg once every 4 weeks. In some embodiments, the fusion proteins or component peptides described herein or the nucleic acid molecules, or the expression vectors that encode them are administered subcutaneously at a dose of 150 mg once every 4 weeks. In some embodiments, the fusion proteins or component peptides described herein or the nucleic acid molecules, or the expression vectors that encode them are administered subcutaneously at a dose of at least 150 mg once every 4 weeks. In some embodiments, the fusion proteins or component peptides described herein or the nucleic acid molecules, or the expression vectors that encode them are administered as a fixed dose subcutaneously of about 150 mg once every 4 weeks. In some embodiments, the fusion proteins or component peptides described herein or the nucleic acid molecules, or the expression vectors that encode them are administered as a fixed dose subcutaneously of 150 mg once every 4 weeks. In some embodiments, the fusion proteins or component peptides described herein or the nucleic acid molecules, or the expression vectors that encode them are administered as a fixed dose subcutaneously of at least 150 mg once every 4 weeks. In some embodiments, the fusion proteins or component peptides described herein or the nucleic acid molecules, or the expression vectors that encode them are administered as a fixed dose subcutaneously of about 300 mg once every 4 weeks. In some embodiments, the fusion proteins or component peptides described herein or the nucleic acid molecules, or the expression vectors that encode them are administered as a fixed dose subcutaneously of 300 mg once every 4 weeks. In some embodiments, the fusion proteins or component peptides described herein or the nucleic acid molecules, or the expression vectors that encode them are administered as a fixed dose subcutaneously of about 600 mg once every 4 weeks. In some embodiments, the fusion proteins or component peptides described herein or the nucleic acid molecules, or the expression vectors that encode them are administered as a fixed dose subcutaneously of 600 mg once every 4 weeks.

In some embodiments, the fusion proteins or component peptides described herein or the nucleic acid molecules, or the expression vectors that encode them are administered intravenously at a dose of about 0.3 mg/kg once every 1 month. In some embodiments, the fusion proteins or component peptides described herein or the nucleic acid molecules, or the expression vectors that encode them are administered intravenously at a dose of 0.3 mg/kg once every 1 month. In certain embodiments, the fusion proteins or component peptides described herein or the nucleic acid molecules, or the expression vectors that encode them are administered at about 0.3 mg/kg once every 1 month. In certain embodiments, the fusion proteins or component peptides described herein or the nucleic acid molecules, or the expression vectors that encode them are administered at 0.3 mg/kg once every 1 month. In certain embodiments, the fusion proteins or component peptides described herein or the nucleic acid molecules, or the expression vectors that encode them are administered at about 1 mg/kg once every 1 month. In certain embodiments, the fusion proteins or component peptides described herein or the nucleic acid molecules, or the expression vectors that encode them are administered at 1 mg/kg once every 1 month. In certain embodiments, the fusion proteins or component peptides described herein or the nucleic acid molecules, or the expression vectors that encode them are administered at about 3 mg/kg once every 1 month. In certain embodiments, the fusion proteins or component peptides described herein or the nucleic acid molecules, or the expression vectors that encode them are administered at 3 mg/kg once every 1 month. In certain embodiments, the fusion proteins or component peptides described herein or the nucleic acid molecules, or the expression vectors that encode them are administered at about 10 mg/kg once every 1 month. In certain embodiments, the fusion proteins or component peptides described herein or the nucleic acid molecules, or the expression vectors that encode them are administered at 10 mg/kg once every 1 month.

In some embodiments, the fusion proteins or component peptides described herein or the nucleic acid molecules, or the expression vectors that encode them are administered at a dose of about 150 mg once every 1 month. In some embodiments, the fusion proteins or component peptides described herein or the nucleic acid molecules, or the expression vectors that encode them are administered at a dose of 150 mg once every 1 month. In some embodiments, the fusion proteins or component peptides described herein or the nucleic acid molecules, or the expression vectors that encode them are administered at a dose of at least 150 mg once every 1 month. In some embodiments, the fusion proteins or component peptides described herein or the nucleic acid molecules, or the expression vectors that encode them are administered as a fixed dose of about 150 mg once every 1 month. In some embodiments, the fusion proteins or component peptides described herein or the nucleic acid molecules, or the expression vectors that encode them are administered as a fixed dose of 150 mg once every 1 month. In some embodiments, the fusion proteins or component peptides described herein or the nucleic acid molecules, or the expression vectors that encode them are administered as a fixed dose of at least 150 mg once every 1 month. In some embodiments, the fusion proteins or component peptides described herein or the nucleic acid molecules, or the expression vectors that encode them are administered as a fixed dose of about 300 mg once every 1 month. In some embodiments, the fusion proteins or component peptides described herein or the nucleic acid molecules, or the expression vectors that encode them are administered as a fixed dose of 300 mg once every 1 month. In some embodiments, the fusion proteins or component peptides described herein or the nucleic acid molecules, or the expression vectors that encode them are administered as a fixed dose of about 600 mg once every 1 month. In some embodiments, the fusion proteins or component peptides described herein or the nucleic acid molecules, or the expression vectors that encode them are administered as a fixed dose of 600 mg once every 1 month.

In some embodiments, the fusion proteins or component peptides described herein or the nucleic acid molecules, or the expression vectors that encode them are administered subcutaneously at a dose of about 150 mg once every 1 month. In some embodiments, the fusion proteins or component peptides described herein or the nucleic acid molecules, or the expression vectors that encode them are administered subcutaneously at a dose of 150 mg once every 1 month. In some embodiments, the fusion proteins or component peptides described herein or the nucleic acid molecules, or the expression vectors that encode them are administered subcutaneously at a dose of at least 150 mg once every 1 month. In some embodiments, the fusion proteins or component peptides described herein or the nucleic acid molecules, or the expression vectors that encode them are administered as a fixed dose subcutaneously of about 150 mg once every 1 month. In some embodiments, the fusion proteins or component peptides described herein or the nucleic acid molecules, or the expression vectors that encode them are administered as a fixed dose subcutaneously of 150 mg once every 1 month. In some embodiments, the fusion proteins or component peptides described herein or the nucleic acid molecules, or the expression vectors that encode them are administered as a fixed dose subcutaneously of at least 150 mg once every 1 month. In some embodiments, the fusion proteins or component peptides described herein or the nucleic acid molecules, or the expression vectors that encode them are administered as a fixed dose subcutaneously of about 300 mg once every 1 month. In some embodiments, the fusion proteins or component peptides described herein or the nucleic acid molecules, or the expression vectors that encode them are administered as a fixed dose subcutaneously of 300 mg once every 1 month. In some embodiments, the fusion proteins or component peptides described herein or the nucleic acid molecules, or the expression vectors that encode them are administered as a fixed dose subcutaneously of about 600 mg once every 1 month. In some embodiments, the fusion proteins or component peptides described herein or the nucleic acid molecules, or the expression vectors that encode them are administered as a fixed dose subcutaneously of 600 mg once every 1 month.

### Kits

Any of the compositions described herein may be comprised in a kit. In a non-limiting example, the kit comprises one or more of the fusion proteins or component peptides described herein or the nucleic acid molecules, or the expression vectors that encode them.

The kit may further include reagents or instructions for using the fusion proteins or component peptides described herein or the nucleic acid molecules, or the expression vectors that encode them in a subject. It may also include one or more buffers.

The components of the kits may be packaged either in aqueous media or in lyophilized form. The container means of the kits will generally include at least one vial, test tube, flask, bottle, syringe, or other container means, into which a component may be placed, and preferably, suitably aliquoted. Where there is more than one component in the kit (labeling reagent and label may be packaged together), the kit also will generally contain a second, third, or other additional container into which the additional components may be separately placed. The kits may also comprise a second container means for containing a sterile, pharmaceutically acceptable buffer and/or other diluent. However, various combinations of components may be comprised in a vial. The kits of the present disclosure also typically include a means for containing the fusion proteins or component peptides described herein or the nucleic acid molecules, or the expression vectors that encode them, and any other reagent containers in close confinement for commercial sale.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The examples of the present disclosure are offered by way of illustration and explanation, and are not intended to limit the scope of the present disclosure. The results described in each Example are reflective of the particular conditions outlined in the experiments described therein.

### Example 1. Heparin Chromatography for Relaxin-2 Fusion Protein Analogs

Heparin chromatography is a method that can be used at early candidate screening to better understand a molecule's propensity to interact with elements of the vasculature when dosed in patients. Heparin and heparin sulfate proteoglycans are negatively charged polysaccharides present in vasculature and in tissues, of which positively charged molecules may bind at physiological pH (i.e., pI>7.4). Here, heparin chromatography was employed to screen for candidates/variants with reduced heparin binding, which is predictive of good PK properties. Materials used for the heparin chromatography are provided in Table 11.

TABLE 11
Materials

| Item | Vendor | Cat No. |
|---|---|---|
| POROS ™ Heparin | Thermo Fisher | 4333411 |
| 2.1 × 30 mm Column | | |

### Methods

Mobile Phase A (Binding): 20 mM Tris pH 7.4; Mobile Phase B (Elution): 20 mM Tris pH 7.4+1M NaCl; Injection: 10 μg; Detection: 220 nm.
1. Equilibrated heparin column using mobile phase A for 10 minutes at 0.5 mL/min prior to analysis.
2. Diluted samples for analysis to 1 mg/mL with 20 mM Tris pH 7.4 to minimize ionic strength.
3. Ran the Heparin Chromatography method on the Agilent HPLC, using gradient shown in Table 12, below:

TABLE 12
HPLC Gradient

| Time (min) | Flow (mL/min) | % A | % B |
|---|---|---|---|
| 0 | 0.5 | 100 | 0 |
| 1 | 0.5 | 100 | 0 |
| 6 | 0.5 | 50 | 50 |
| 6.5 | 0.5 | 50 | 50 |
| 7 | 0.5 | 0 | 100 |
| 8 | 0.5 | 0 | 100 |
| 8.5 | 0.5 | 100 | 0 |
| 10 | 0.5 | 100 | 0 |

4. Included a positive control (no Heparin binding, Human IgG pool) and negative control (SE301 or AT1R).
5. Analyzed samples for retention time and reported relative retention time compared to the positive control (i.e., RT sample/RT positive control).
6. Calculated the approximate concentration of NaCl needed to elute using the following calculation:

[NaC]ᵢ=(RTsampl−1)*100

The results of the calculation are shown in Table 13.

TABLE 13
Retention Time, Relative Retention Time, and
NaCl Concentration for Samples

| Sample | RT | RRT | [NaCl] |
|---|---|---|---|
| Positive Control | 1.5 | N/A | 50 |
| Negative Control | 3.5 | 2.3 | 250 |
| Sample | 2.0 | 1.3 | 100 |

### Results

Table 14 shows the results of the heparin chromatography for a variety of relaxin-2 analog fusion proteins.

TABLE 14
Heparin Chromatography

| | | Heparin Chromatography | |
|---|---|---|---|
| | Theoretical | | ~[NaCl] |
| Sample | pI | RT | (mM) at elution |
| IgG | N/A | 2.0 | 20 |
| Prior fusion protein | 8.5 (9.4*) | 4.6 | 278 |
| SEQ ID NO: 300 | 8.2 | 3.9 | 208 |
| SEQ ID NO: 302 | 7.9 | 3.3 | 152 |
| SEQ ID NO: 303 | 7.9 | 3.4 | 163 |
| SEQ ID NO: 305 | 7.6 | 2.7 | 91 |
| SEQ ID NO: 306 | 7.6 | 2.8 | 99 |
| SEQ ID NO: 308 | 7.2 | 2.4 | 62 |
| SEQ ID NO: 309 | 7.2 | 2.5 | 70 |
| SEQ ID NO: 310 | 6.8 | 2.2 | 39 |
| SEQ ID NO: 311 | 6.8 | 2.2 | 43 |
| SEQ ID NO: 359 | 8.4 | 3.6 | 183 |
| SEQ ID NO: 360 | 8.3 | 3.2 | 140 |

*Experimentally determined using imaged capillary isoelectric focusing.

IgG is from Jackson ImmunoResearch (Catalog #009-000-003). The “Prior fusion protein” is a LALA IgG-RelB-Linker-RelA fusion with a theoretical pI of 8.5, but an experimentally determined pI of 9.4. Its linker protein comprises only one acidic amino acid. SEQ ID NOs: 300, 302, 303, 305, 306, and 308-311 have linker proteins comprising at least two acidic amino acids as well as LALA IgG (SEQ ID NO: 77 or 81). The final two fusion proteins have linker proteins comprising only one acidic amino acid and have higher theoretical pI's. As shown in Table 14, above, there is a correlation between lower pI and lower non-specific binding found through heparin chromatography.

### Example 2. Low pI Relaxin-2 Fusion Protein Analogs Tend to have Decreased Self Association as Measured by Affinity-Capture Self-Interaction Nanoparticle Spectroscopy (AC-SINS)

Understanding a molecule's propensity to self-associate is critical when evaluating biophysical properties of a development candidate. There are numerous ways to evaluate a molecule's propensity to self-associate, concentrating the molecule to high concentrations and evaluating by SEC (% Monomer) or measuring changes in turbidity (OD 340 nm), using DLS to calculate the second virial coefficient (B₂₂) or self-interaction coefficient (k_{d}), or using AC-SINS (Δλₘₐₓ). All three of these methods provide useful information but use different amounts of material to perform the evaluation. AC-SINS has emerged as a high throughput method for evaluating self-association using minimal material but still giving locally high concentrations by using affinity capture on gold nanoparticles. In short, gold nanoparticles are pre-coated with anti-human antibodies (Fc, Fab and H+L), which when incubated with target antibodies in dilute solutions, capture and concentrate in solution the antibody of interest. When the immobilized molecules of interest interact, the inter-particle distances decrease between gold nanoparticles, leading to increased plasmon wavelengths (i.e., red shift) that can be quantified using UV-VIS spectroscopy. Materials used for the spectroscopy are provided in Table 15.

TABLE 15
Materials

| Item | Vendor | Cat No. |
|---|---|---|
| 1M sodium acetate pH 4.3 | Molecular | MD2-019-PH |
| | Dimensions | |
| 1x DPBS | Gibco | 14190-136 |
| Panitumumab (Low Assoc Ctrl) | MyBioSource | MBS156169 |
| Ipilimumab (Med Assoc Ctrl) | MyBioSource | MBS156153 |
| Ganitumab (High Assoc Ctrl) | MyBioSource | MBS156142 |
| 20 nm gold nanoparticles | Ted Pella | 15705 |
| PEG methyl ether thiol (2 kDa) | Sigma | 729140 |
| Goat anti-Human IgG, Fcy | Jackson | 109-005-098 |
| | ImmunoResearch | |
| Goat non-specific mAb | Jackson | 005-000-003 |
| | ImmunoResearch | |
| Zeba Desalting Columns 40K 5 mL | Thermo Fisher | 87770 |
| Zeba Desalting Columns 40K 2 mL | Thermo Fisher | 87768 |
| Zeba Desalting Columns 40K | Thermo Fisher | 87766 |
| 0.5 mL | | |
| Costar 384-well Polystyrene plates | Fisher Scientific | 12-565-506 |
| 96-well Polypropylene plates | Grenier Bio-One | 652230P |

### Methods

Preparing Buffer Solutions: To prepare 20 mM sodium acetate pH 4.3, 2 mL 1M sodium acetate pH 4.3 stock was diluted to 100 mL with MilliQ water. pH was measured 4.3±0.1 and the solution was sterile filtered. The solution remained stable at room temperature for 1 month. To 1 g PEG methyl ether thiol, was added 10 mL MilliQ water. This was vortexed briefly to suspend solids, making a 50 mM solution. To prepare a 10 μM solution for final dilution, the dilution scheme below was followed:
a. Dilute 50 mM stock to 1 mM (20 μL 50 mM stock+980 μL MilliQ water)
b. Dilute 1 mM step to 100 μM (10 μL 1 mM stock+90 μL MilliQ water)
c. Dilute 100 μM step to 10 μM (100 μL 100 μM stock+900 μL MilliQ water)
d. Volumes can be scaled according to number of samples to assay
e. Remaining 50 mM stock should be aliquoted and kept at −20° C. until needed

Preparing Gold NanoParticle Solution: Goat anti-human Fc IgG antibody (capture) and goat IgG antibody (non-capture) were buffer exchanged into 20 mM sodium acetate, pH 4.3. After buffer exchange, concentrations were normalized to 0.4 mg/mL for both antibodies. A 4:1 volume ratio mixture of capture (anti-Fc): non-capture (Goat IgG) solution was prepared for 80% capture capacity coating solution to be used to incubate gold nanoparticles (AuNPs).

A 9:1 volume ratio of AuNPs: coating solution was made. The solution was incubated at room temperature, overnight in the dark. After incubation, thiolated PEG was added to 0.1 μM final concentration from the diluted 10 μM stock to block empty sites on the AuNPs (i.e., 5 mL solution of AuNPs, add 50 μL 10 μM stock) and incubated at RT for one hour in the dark.

Preparing AuNP Solution: 2 mL of coated AuNP solution was centrifuged at 20,000×g for 15 minutes to sediment the AuNPs and 1800 μL supernatant was carefully removed using a 1 mL pipette. The pelleted AuNPs were gently resuspended using a 200 μL pipette to generate a 10× concentrated stock of coated AuNPs.

Preparing Target Antibody Solution (either method follows this): For each sample analyzed, 10 μL of AuNP concentrate was incubated with 100 μL antibody test solution (normalize to 0.05 mg/mL) at room temperature in the dark for 2 hours in a 96-well polypropylene plate. Two blank solutions were prepared with 10 μL 10×AuNP concentrated to 100 μL PBS for purposes of blanking the assay and determining wavelength shift upon addition of test antibody. Ganitumab was included as a positive control (high association, red shift) and Panitumumab as a negative control (no association, no UV shift). Each sample was prepared in duplicate for analysis. After the 2-hour incubation, 100 μL of resulting solution was transferred to a UV transparent polystyrene plate (384-well format). Two blank solutions were transferred to properly assess wavelength shift, then add duplicate standards and samples for analysis. The plate was then centrifuged for 1 minute at 1000×g to level the solutions in the wells. Absorbance data were collected from 510 to 570 nm in 2 nm steps to determine wavelength shifts for each sample relative to AuNPs alone.

### Results

The results from ASCINS are shown below in Table 16.

TABLE 16
pI Variants Have Decreased Self-Association Propensity

| | Isoelectric Point | |
|---|---|---|
| Sample | (Calculated) | Δλₘₐₓ |
| Prior fusion protein (Control) | 8.5 | 15.2 |
| Prior fusion protein (LALA) | 8.5 | 15.9 |
| SEQ ID NO: 300 | 8.2 | 7.5 |
| SEQ ID NO: 301 | 7.9 | 1.2 |
| SEQ ID NO: 302 | 7.9 | 7.0 |
| SEQ ID NO: 303 | 7.9 | 6.7 |
| SEQ ID NO: 305 | 7.6 | 1.8 |
| SEQ ID NO: 306 | 7.6 | 1.9 |
| SEQ ID NO: 308 | 7.2 | −0.6 |
| SEQ ID NO: 309 | 7.2 | −0.4 |
| SEQ ID NO: 310 | 6.8 | 0.4 |
| SEQ ID NO: 311 | 6.8 | 0.4 |
| SEQ ID NO: 359 | 8.4 | 10.1 |
| SEQ ID NO: 360 | 8.3 | 6.1 |

As shown in Table 16, above, fusion proteins with low pI also have a tendency to show low self-aggregation.

### Example 3. Relaxin-2 Fusion Protein Analogs Induce cAMP Response in RXFP1 Transfected Cells

This Example provides data relating to the potency of various relaxin-2 fusion protein analogs described herein. Potency of the fusion protein analogs were assayed by testing their ability to activate RXFP1 by measuring cAMP signaling.

### Methods

HEK293 cells were seeded into a 96-well tissue culture plate followed by transient co-transfection with a human RXFP1 and a pGloSensor-22F plasmid. Transfected cells were stimulated by relaxin-2 or fusion protein analogs thereof, inducing Gs-mediated cAMP signaling. cAMP is assayed using the activity of the GloSensor biosensor, which is a mutant luciferase fused to a cAMP binding domain, leading to a production of light in the presence of its substrate luciferin. This readout of relative luminescent units (RLU) is used a proxy for cAMP response.

### Reagents

96-well tissue-culture treated plates. White with clear bottom. (Corning #3610)
HEK293 cells (ATCC CRL-1573)
Poly-D-lysine (Gibco A3890401)
DPBS (No calcium, no magnesium; Gibco 14190250)
DMEM (High glucose with L-glutamine and Sodium Pyruvate; Gibco 11995065)
TrypLE Express (Gibco 12605010)
FBS (HyClone™, Australian origin; Cytiva SH30084)
Penicillin-Streptomycin (Gibco 15140122)
CO₂-independent media (Gibco 18045088)
Opti-MEM™ I Reduced Serum media (Gibco 31985062)
pGloSensor™-22F cAMP plasmid (Promega Cat. #E2301)
D-luciferin, Potassium Salt (GoldBio LUCK-1G)
FuGENE HD transfection reagent (Promega #E2311)
Reservoirs (Corning/Axygen RES-V-25-SI)
Relaxin-2 (R&D Biosystems 6586-RN-025)
RXFP1-containing plasmid (pcDNA5/FRT/TO-human RXFP1, full-length)
Forskolin (Sigma F6886)
Plate reader capable of reading luminescence (CLARIOstar Plus)

### Reagent Preparation

D-luciferin, Potassium Salt: D-luciferin was reconstituted in 10 mM HEPES, pH 7.5 at 25 mg/mL (78.5 mM; MW=318.4). This was aliquoted into single-use aliquots of ˜200-500 μL in sterile microfuge tubes and stored at −80° C.

Relaxin-2 peptide: Relaxin-2 peptides or relaxin-2 fusion protein analogs were reconstituted at 0.1 mg/mL in sterile DPBS (MW=5,986 Da, ε=12,865 M⁻¹ cm⁻¹) and measured at A₂₈₀ to determine final concentration. Aliquots were stored at −20° C.

Forskolin: Forskolin was reconstituted in 100% DMSO at 5 mM (2.05 mg/mL, MW=410.5). Aliquots were stored at −20° C.

cAMP assay media: CO₂-independent media was pre-warmed to 37° C. using the bead bath. A single aliquot of D-luciferin was thawed and added at 5% final concentration (e.g., 4.75 mL cAMP assay media+250 μL of D-luciferin stock; gives 1.25 mg/mL or 3.93 mM final D-luciferin). This was used within the same day or discarded.

### Cell Culture and Maintenance

HEK293 cells (ATCC CRL-1573) were cultured in DMEM+10% FBS, 1% (1X or 10 U/mL) Pen-Strep in a humidified CO₂ incubator at 37 C, 5% CO₂ until 80-100% confluency. Cells were typically split 1:6 for 3 days and maintained in a sterile T-75 tissue culture flask.

cAMP Signaling Assay Protocol

This protocol was adapted from the GloSensor cAMP assay by Promega.

Raw data was exported to Excel using the MARS data analysis software that is opened following a run on the CLARIOstar plate reader. These values are measured in RLU, or relative luminescence units.

As shown in Table 17, all of the low pI relaxin-2 fusion protein analogs were able to induce a cAMP response in RXFP1 transfected cells.

TABLE 17
cAMP Response in RXFP1 Transfected HEK293 Cells

| Agonist | pEC₅₀ | EC₅₀ in nM |
|---|---|---|
| Relaxin | 10.38 | 0.042 |
| Prior fusion protein (Control) | 9.51 | 0.31 |
| Prior fusion protein (LALA) | 9.49 | 0.33 |
| SEQ ID NO: 300 | 8.66 | 2.17 |
| SEQ ID NO: 301 | 8.10 | 7.87 |
| SEQ ID NO: 302 | 8.62 | 2.41 |
| SEQ ID NO: 303 | 8.72 | 1.90 |
| SEQ ID NO: 305 | 7.83 | 14.7 |
| SEQ ID NO: 306 | 7.75 | 17.7 |
| SEQ ID NO: 308 | 7.25 | 56.7 |
| SEQ ID NO: 309 | 7.18 | 65.8 |
| SEQ ID NO: 310 | 6.76 | 173.3 |
| SEQ ID NO: 311 | 7.02 | 96.2 |
| SEQ ID NO: 359 | 9.37 | 0.42 |
| SEQ ID NO: 360 | 8.84 | 1.46 |

### Example 4. In Vitro Characteristics of Relaxin-2 Fusion Protein Analogs

This Example provides in vitro characteristics of various relaxin-2 fusion protein analogs described herein.

### Methods

Heparin chromatography: heparin chromatography was performed to understand the propensity of a relaxin-2 fusion protein analog to interact with elements of the vasculature and/or rapidly distribute into tissues when dosed in patients. Analogs that were found to bind heparin weakly may be predictive of good pharmacokinetic properties. Briefly, a heparin column was equilibrated using mobile phase A (20 mM Tris pH 7.4) for 10 minutes at 0.5 mL/min prior to analysis. 10 μg per sample was run using the Heparin Chromatography method on an Agilent HPLC using 280 nm detection, using gradient shown in Table 18, below (mobile phase B: 20 mM Tris pH 7.4, 1 M NaCl).

TABLE 18
HPLC Gradient

| Time (min) | Flow (mL/min) | % A | % B |
|---|---|---|---|
| 0 | 0.5 | 100 | 0 |
| 6 | 0.5 | 50 | 50 |
| 7 | 0.5 | 0 | 100 |
| 8 | 0.5 | 0 | 100 |
| 8.5 | 0.5 | 100 | 0 |
| 10 | 0.5 | 100 | 0 |

A positive control (no Heparin binding, pembrolizumab) and negative control (mild Heparin binding, adalimumab) was included, and samples were analyzed for retention time and relative retention time compared to the positive control (i.e., RT sample/RT positive control). The approximate concentration of NaCl needed to elute was calculated using the following calculation:

$$[ NaC \text{?} = ( RTsamp \text{?} * 100$$

$$\text{?} \text{indicates text missing or illegible when filed}$$

The results of the calculation are shown in Table 19.

TABLE 19
Retention Time and NaCl Concentration for Samples

| Sample | RT | [NaCl] |
|---|---|---|
| Positive Control | 1.5 | 150 |
| Negative Control | 3.5 | 350 |
| Sample | 2.0 | 200 |

Hydrophobic Interaction Chromatography (HIC): HIC is a chromatography method that separates molecules based on their hydrophobicity. To a butyl HIC column pre-equilibrated with high ammonium sulfate buffer, 10 μg of protein was injected. The protein was eluted with a gradient from high salt concentration to low salt concentration over ten minutes. Samples were compared for hydrophobicity based on retention time, with high retention time indicative of high hydrophobicity and low retention time indicative of low hydrophobicity. Retention times were converted to approximate salt concentration at elution and compared against high and low hydrophobicity standards.

Size exclusion chromatography (SEC): SEC is a liquid chromatography method used to determine levels of monomeric and multimeric species in solution for a given analyte. SEC was used to assess the presence of fusion protein aggregates. Samples were prepared and added to a 1.7 μm particle SEC column with an aqueous mobile phase comprised of 25 mM potassium phosphate and 0.5M potassium chloride pH 8.0. Once elution of sample was verified, the method was capable of quantifying levels of soluble aggregate species in the sample, with high resolution between peaks of monomer and high molecular weight (HMW) species. Percentage of monomer (i.e., % monomer) and other species (e.g., HMW species, low molecular weight species) was calculated by integrating the corresponding elution curve to determine the percent area.

Capillary isoelectric focusing (cIEF): Imaged cIEF was used to separate differentially charged molecules (i.e., relaxin-2 fusion protein analogs) using electrophoretic mobility in an ampholyte solution to determine their isoelectric points (pI). Molecules were loaded to a capillary and separated based on their pI by allowing molecules to migrate along an electrical field until the molecules reached the pH corresponding to their pI. UV absorption of the whole capillary was measured throughout the separation, which allowed for real-time observation as well as final quantification.

Baculovirus Particle (BVP) ELISA: BVP ELISA was employed to understand the propensity of a relaxin-2 fusion protein analog for non-specific or non-target interactions. BVPs are empty viral capsids with no viral genome, but in the process of production, budding off from the cell membrane allows them to take components of the cell membrane along with them. Thus, the BVPs possess a highly diverse cell surface with many moieties present, which mimic what the molecule of interest (i.e., relaxin-2 fusion protein analog) may encounter in vivo. Briefly, BVPs are coated on a plate by adding 25 μL of BVP solution to each well. BVP solution was made by diluting BVP stock (Medna Scientific; Cat. No. E3001) to 1×10⁶ PFU/mL with 0.1 M carbonate buffer, pH 9.6. Following overnight incubation at 5° C., BVP solution was blotted from wells and wells were washed three times with PBST. Plates were blocked with 100 μL/well of 1×BSA in PBS blocking buffer (Cepham Life Sciences; Cat. No. 10615). Plates were incubated at 25° C. on a plate shaker for 1 hour. Blocking solution was blotted from wells and wells were washed three times with PBST. Samples (i.e., relaxin-2 fusion protein analogs) were prepared in duplicate to cover dilution range from 3 μM to 0.1 nM and added to plates. Plates were incubated at 25° C. for 1 hour, after which the wells were blotted and washed three times with PBST. 25 μL/well of 1:10,000 diluted detection monoclonal antibody (Peroxidase AffiniPure Goat Anti-Human IgG, Fcγ fragment specific; Jackson ImmunoResearch; Cat. No. 50-194-1564) was added, and plates were incubated at 25° C. for 1 hour, after which the wells were blotted and washed three times with PBST. 1-Step™ Ultra TMB-ELISA Substrate Solution (Life Technologies; Cat. No. 34029) was then added. After about 2 minutes, 25 μL 2N HCl was added to quench the reaction, and a plate reader was used to analyze the plate at 450 nm with correction at 570 nm.

Potency assay: HEK293 cells were seeded into a 96-well tissue culture plate followed by transient co-transfection with a human RXFP1 and a pGloSensor-22F plasmid. Transfected cells were stimulated by relaxin-2 or fusion protein analogs thereof, inducing Gs-mediated cAMP signaling. cAMP is assayed using the activity of the GloSensor biosensor, which is a mutant luciferase fused to a cAMP binding domain, leading to a production of light in the presence of its substrate luciferin. This readout of relative luminescent units (RLU) is used as a proxy for cAMP response.

cAMP Signaling Assay Protocol: this protocol is adapted from the GloSensor cAMP assay by Promega. Raw data was exported to Excel using the MARS data analysis software that is opened following a run on the CLARIOstar plate reader. These values are measured in RLU, or relative luminescence units.

Affinity-Capture Self-Interaction Nanoparticle Spectroscopy (AC-SINS): AC-SINS was performed to understand the propensity of a molecule (i.e., relaxin-2 fusion protein analog) to self-associate. Briefly, gold nanoparticles were pre-coated with anti-human antibodies (Fc, Fab and H+L), which when incubated with target antibodies in dilute solutions, capture and concentrate in solution the antibody of interest. When the immobilized molecules of interest interact, the inter-particle distances decrease between gold nanoparticles, leading to increased plasmon wavelengths (i.e., red shift) that can be quantified using UV-VIS spectroscopy. Materials used for the spectroscopy are provided in Table 20.

TABLE 20
Materials for AC-SINS

| Item | Vendor | Cat No. |
|---|---|---|
| 1M sodium acetate pH 4.3 | Molecular | MD2-019-PH |
| | Dimensions | |
| 1x DPBS | Gibco | 14190-136 |
| Panitumumab (Low Assoc Ctrl) | MyBioSource | MBS156169 |
| Ipilimumab (Med Assoc Ctrl) | MyBioSource | MBS156153 |
| Ganitumab (High Assoc Ctrl) | MyBioSource | MBS156142 |
| 20 nm gold nanoparticles | Ted Pella | 15705 |
| PEG methyl ether thiol (2 kDa) | Sigma | 729140 |
| Goat anti-Human IgG, Fcγ | Jackson | 109-005-098 |
| | ImmunoResearch | |
| Goat non-specific mAb | Jackson | 005-000-003 |
| | ImmunoResearch | |
| Zeba Desalting Columns 40K 5 mL | Thermo Fisher | 87770 |
| Zeba Desalting Columns 40K 2 mL | Thermo Fisher | 87768 |
| Zeba Desalting Columns 40K 0.5 | Thermo Fisher | 87766 |
| mL | | |
| Costar 384-well Polystyrene plates | Fisher Scientific | 12-565-506 |
| 96-well Polypropylene plates | Grenier Bio-One | 652230P |

Goat anti-human Fc IgG antibody (capture) and goat IgG antibody (non-capture) were buffer exchanged into 20 mM sodium acetate, pH 4.3. After buffer exchange, concentrations were normalized to 0.4 mg/mL for both antibodies. A 4:1 volume ratio mixture of capture (anti-Fc): non-capture (Goat IgG) solution was prepared for 80% capture capacity coating solution to be used to incubate gold nanoparticles (AuNPs). A 9:1 volume ratio of AuNPs: coating solution was made. The solution was incubated at room temperature, overnight in the dark. After incubation, thiolated PEG was added to 0.1 μM final concentration from the diluted 10 μM stock to block empty sites on the AuNPs (i.e., 5 mL solution of AuNPs, add 50 μL 10 μM stock) and incubated at RT for one hour in the dark.

2 mL of coated AuNP solution was centrifuged at 20,000×g for 15 minutes to sediment the AuNPs and 1800 μL supernatant was carefully removed using a 1 mL pipette. The pelleted AuNPs were gently resuspended using a 200 μL pipette to generate a 10× concentrated stock of coated AuNPs. For each sample analyzed, 5 μL of AuNP concentrate was incubated with 45 μL antibody test solution (normalized to 0.05 mg/mL) at room temperature in the dark for 2 hours in a 384-well polypropylene plate. After the 2-hour incubation, absorbance data was collected from 450 nm to 650 nm in 1 nm steps to determine wavelength shifts for each sample relative to AuNPs alone.

Nanoscale Differential Scanning Fluorimetry (NanoDSF): NanoDSF was performed using the NanoTemper Prometheus Panta to investigate the conformational stability of a relaxin-2 protein fusion analog. Conformational stability was measured by applying a thermal ramp to a solution containing the molecule of interest, measuring the intrinsic fluorescence, backscattering, and using dynamic light scattering (DLS) to provide various thermal stability parameters, including the temperature at which fusion protein unfolding begins (Tₒₙₛₑₜ), the temperature at which half of the fusion protein in a given sample is unfolded (Tₘ1), and the temperature at which fusion protein aggregation begins (T_{agg}).

Sequences: Sequences of relaxin-2 fusion protein analogs are described throughout the present disclosure. SEQ ID NOs: 496, 497, and 501 are set forth below:

(SEQ ID NO: 496)
DKTHTCPPCPAPEAAGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALAAPIEKTISKAKGQPREPQVYTLPPSRDELTKNQVS
LTCLVKGFYPSDIAVEWESNGQPENNYKTTPPVLDSDGSFFLYSKLTV
DKSRWQQGNVFSCSVLHEALHSHYTQKSLSLSPGKGGSDSWKEEVIKL
CGRELVRAQIAICGKSTASDAAGANANAGARQLYSALANKCCHVGCTK
RSLARFC;
(SEQ ID NO: 497)
DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVS
LTCLVKGFYPSDIAVEWESNGQPENNYKTTPPVLDSDGSFFLYSKLTV
DKSRWQQGNVFSCSVMHEALHNHYTQKSLSLSPGKGGGGSGGGGSGGG
GSQLYSALANKCCHVGCTKRSLARFCGGGGSGGGGSGGGGSSWMEEVI
KLCGRELVRAQIAICGMSTWS;
and
(SEQ ID NO: 501)
MPRLFFFHLLGVCLLLNQFSRAVADSWMEEVIKLCGRELVRAQIAICG
MSTWSKRSLSQEDAPQTPRPVAEIVPSFINKDTETINMMSEFVANLPQ
ELKLTLSEMQPALPQLQQHVPVLKDSSLLFEEFKKLIRNRQSEAADSS
PSELKYLGLDTHSRKKRQLYSALANKCCHVGCTKRSLARFC.

### Results

The results are shown below in Tables 21, 22, and 23.

TABLE 21
In vitro Characteristics of Relaxin-2 Fusion Protein Analogs.

| | Heparin | | cIEF | BVP | | | | |
|---|---|---|---|---|---|---|---|---|
| | Binding | | Isoelectric | ELISA | | AC- | NanoDSF | |
| | | [NaCl] | Point | Normalized | Potency³ | SINS | Tₒₙₛₑₜ | Tₘ1 |
| Sample | RT¹ | (mM)² | (pI) | Score | (nM) | Δλmax | (° C.) | (° C.) |
| Wild-type | ND | ND | 9.4 | N/A | 0.1 | N/A | N/A | N/A |
| human | | | | | | | | |
| relaxin-2 | | | | | | | | |
| SEQ ID NO: | ND | ND | 9.0 | 28 | 0.3 | 7 | 61.4 | 68.9 |
| 497 | | | | | | | | |
| SEQ ID NO: | 4.75 | 400 | 9.1 | 34 | 0.2 | 14 | 59.7 | 68.4 |
| 496 | | | | | | | | |
| SEQ ID NO: | 3.88 | 330 | 8.9 | 2 | 2.5 | 8 | 60.5 | 70.4 |
| 361 | | | | | | | | |
| SEQ ID NO: | NT | NT | ND | ND | 12.3 | 1 | 63.3 | 70.7 |
| 362 | | | | | | | | |
| SEQ ID NO: | 3.32 | 280 | 8.5 | 1 | 2.9 | 7 | 62.8 | 70.6 |
| 363 | | | | | | | | |
| SEQ ID NO: | 3.43 | 290 | 8.5 | 1 | 2.5 | 7 | 61.6 | 70.6 |
| 304 | | | | | | | | |
| SEQ ID NO: | 2.71 | 230 | 7.9 | 1 | 16.0 | 2 | 62.9 | 70.9 |
| 364 | | | | | | | | |
| SEQ ID NO: | 2.79 | 240 | 7.9 | 1 | 15.8 | 2 | 63.0 | 71.0 |
| 307 | | | | | | | | |
| SEQ ID NO: | 2.42 | 210 | 7.5 | 1 | 60.3 | 0 | 63.9 | 71.1 |
| 365 | | | | | | | | |
| SEQ ID NO: | 2.5 | 210 | 7.1 | 1 | 62.8 | 0 | 63.6 | 71.2 |
| 366 | | | | | | | | |
| SEQ ID NO: | 2.19 | 190 | 7.1 | 1 | 140.0 | 0 | 62.4 | 71.0 |
| 367 | | | | | | | | |
| SEQ ID NO: | 2.23 | 190 | ND | 1 | 101.2 | 0 | 62.8 | 70.8 |
| 368 | | | | | | | | |
| SEQ ID NO: | 3.63 | 310 | ND | 1 | 0.5 | 10 | 62.9 | 70.8 |
| 369 | | | | | | | | |
| SEQ ID NO: | 3.2 | 270 | ND | 1 | 1.8 | 6 | 63.0 | 71.2 |
| 370 | | | | | | | | |
| SEQ ID NO: | 2.7 | 230 | 8.0 | 1 | 4.2 | 5 | 63.0 | 69.9 |
| 313 | | | | | | | | |
| SEQ ID NO: | 3 | 250 | 8.0 | 1 | 3.3 | 11 | 61.9 | 69.3 |
| 315 | | | | | | | | |
| SEQ ID NO: | 2.5 | 210 | 7.5 | 1 | 8.0 | 7 | 61.9 | 69.7 |
| 317 | | | | | | | | |
| SEQ ID NO: | 3.1 | 260 | 8.0 | 1 | 5.8 | 4 | 62.6 | 69.6 |
| 319 | | | | | | | | |
| SEQ ID NO: | 2.6 | 220 | 7.4 | 1 | 10.2 | 3 | 62.3 | 69.9 |
| 321 | | | | | | | | |
| SEQ ID NO: | 3 | 250 | 7.4 | ND | ND | ND | ND | ND |
| 323 | | | | | | | | |
| SEQ ID NO: | 2.3 | 200 | 7.1 | 1 | 23.4 | 4 | 60.9 | 69.9 |
| 325 | | | | | | | | |
| SEQ ID NO: | 2.6 | 220 | 7.5 | 1 | 18.9 | 4 | 62.3 | 70.1 |
| 326 | | | | | | | | |
| SEQ ID NO: | 2.8 | 240 | 7.5 | ND | 9.3 | 7 | 60.9 | 69.2 |
| 327 | | | | | | | | |
| SEQ ID NO: | 2.3 | 200 | 7.1 | 1 | 41.9 | 5 | 61.4 | 69.9 |
| 328 | | | | | | | | |
| SEQ ID NO: | 3.1 | 260 | 8.5 | 2 | 1.9 | 8 | 61.1 | 69.3 |
| 84 | | | | | | | | |
| SEQ ID NO: | 3.1 | 260 | 8.5 | 2 | 2.0 | 9 | 60.8 | 69.4 |
| 85 | | | | | | | | |
| SEQ ID NO: | 2.8 | 240 | 7.9 | 3 | 4.0 | 5 | 60.4 | 69.6 |
| 86 | | | | | | | | |
| SEQ ID NO: | 2.8 | 240 | 7.9 | 2 | 5.2 | 6 | 62.0 | 69.7 |
| 87 | | | | | | | | |
| SEQ ID NO: | 3.1 | 260 | 8.5 | 10 | 1.4 | 9 | 60.6 | 69.5 |
| 88 | | | | | | | | |
| SEQ ID NO: | 3.1 | 260 | 8.4 | 3 | 2.5 | 9 | 60.1 | 69.4 |
| 89 | | | | | | | | |
| SEQ ID NO: | 3.5 | 300 | 8.8 | 4 | 0.6 | 15 | 60.8 | 69.4 |
| 90 | | | | | | | | |
| SEQ ID NO: | 2.7 | 230 | 7.4 | 1 | 3.9 | 8 | 61.1 | 69.6 |
| 91 | | | | | | | | |
| SEQ ID NO: | 2.7 | 230 | 7.4 | 1 | 4.3 | 9 | 61.1 | 69.6 |
| 92 | | | | | | | | |
| SEQ ID NO: | 2.4 | 200 | 7.0 | 1 | 11.1 | 5 | 60.7 | 69.5 |
| 93 | | | | | | | | |
| SEQ ID NO: | 2.5 | 210 | 7.0 | 2 | 12.2 | 5 | 59.8 | 69.5 |
| 94 | | | | | | | | |
| SEQ ID NO: | 2.7 | 230 | 7.4 | 3 | 6.8 | 6 | 59.7 | 69.4 |
| 95 | | | | | | | | |
| SEQ ID NO: | 2.8 | 240 | 7.4 | 3 | 9.9 | 7 | 60.3 | 69.3 |
| 96 | | | | | | | | |
| SEQ ID NO: | 3.1 | 260 | 8.0 | 5 | 1.3 | 13 | 60.4 | 69.2 |
| 97 | | | | | | | | |
| SEQ ID NO: | 2.6 | 220 | 7.0 | 2 | 8.8 | 6 | 61.6 | 69.5 |
| 98 | | | | | | | | |
| SEQ ID NO: | 2.6 | 220 | 7.0 | 1 | 9.4 | 8 | 60.6 | 69.3 |
| 99 | | | | | | | | |
| SEQ ID NO: | 2.3 | 200 | 6.8 | 1 | 28.7 | 4 | 60.7 | 69.5 |
| 100 | | | | | | | | |
| SEQ ID NO: | 2.3 | 200 | 6.8 | 1 | 29.4 | 3 | 62.1 | 69.7 |
| 101 | | | | | | | | |
| SEQ ID NO: | 2.6 | 220 | 7.0 | 1 | 7.5 | 8 | 61.7 | 69.6 |
| 102 | | | | | | | | |
| SEQ ID NO: | 2.6 | 220 | 7.0 | 2 | 12.2 | 6 | 61.3 | 69.6 |
| 103 | | | | | | | | |
| SEQ ID NO: | 2.9 | 250 | 7.4 | 3 | 2.8 | 11 | 61.5 | 69.5 |
| 104 | | | | | | | | |
| SEQ ID NO: | 2.5 | 210 | 7.1 | 2 | 14.0 | 4 | 60.4 | 69.4 |
| 106 | | | | | | | | |
| SEQ ID NO: | 2.3 | 200 | 6.8 | 1 | 35.2 | 2 | 61.0 | 69.5 |
| 108 | | | | | | | | |
| SEQ ID NO: | 2.5 | 210 | 7.0 | 2 | 17.2 | 4 | 60.5 | 69.5 |
| 110 | | | | | | | | |
| SEQ ID NO: | 2.8 | 240 | 7.4 | 2 | 3.5 | 6 | 60.4 | 69.6 |
| 111 | | | | | | | | |
| SEQ ID NO: | 2.4 | 200 | 6.8 | 1 | 24.8 | 3 | 60.9 | 69.5 |
| 112 | | | | | | | | |
| SEQ ID NO: | 2.3 | 200 | 6.8 | 2 | 26.0 | 2 | 60.2 | 69.4 |
| 113 | | | | | | | | |
| SEQ ID NO: | 2.3 | 200 | 6.8 | 1 | 25.9 | 3 | 60.6 | 69.5 |
| 116 | | | | | | | | |
| SEQ ID NO: | 2.4 | 200 | 6.8 | 1 | 31.2 | 2 | 60.9 | 69.7 |
| 117 | | | | | | | | |
| SEQ ID NO: | 2.7 | 230 | 7.1 | 2 | 10.1 | 5 | 60.5 | 69.5 |
| 118 | | | | | | | | |

¹Retention time.
²Concentration of NaCl in mM at peak elution from column.
³Potency based on transient hRXFP1 assay described above.
ND—not determined.
N/A—not applicable.

TABLE 22
In vitro Characteristics of Relaxin-2 Fusion Protein Analogs.

| | Heparin | | | | | | | Calculated | |
|---|---|---|---|---|---|---|---|---|---|
| | binding | HIC | SEC | NanoDSF | | | | isoelectric | |
| | [NaCl] | [Salt] | Monomer | Tₒₙₛₑₜ | Tₘ1 | T_{agg} | Potency¹ | point | CIEF |
| Sample | (mM)² | (mM) | (%) | (° C.) | (° C.) | (° C.) | (nM) | (pI) | pI |
| SEQ ID | 217 | 620 | ND | 59 | 69 | 71 | 8.5 | 8 | 7.6 |
| NO: 87 | | | | | | | | | |
| SEQ ID | 225 | 630 | 95.6 | 60 | 70 | 81 | 4.9 | 8 | 7.6 |
| NO: 119 | | | | | | | | | |
| SEQ ID | 225 | 630 | 95.5 | 60 | 70 | 84 | 4.8 | 8 | 7.6 |
| NO: 120 | | | | | | | | | |
| SEQ ID | 225 | 640 | 95.8 | 60 | 70 | 80 | 5.5 | 8 | 7.6 |
| NO: 121 | | | | | | | | | |
| SEQ ID | 225 | 650 | 95.2 | 60 | 69 | 80 | 6.5 | 8 | 7.6 |
| NO: 122 | | | | | | | | | |
| SEQ ID | 200 | 480 | 93.4 | 60 | 69 | 91 | 11.7 | 8 | 7.6 |
| NO: 123 | | | | | | | | | |
| SEQ ID | 192 | 560 | 95.1 | 60 | 69 | 77 | 20.8 | 8 | 7.6 |
| NO: 124 | | | | | | | | | |
| SEQ ID | 192 | 650 | 95.7 | 60 | 69 | 84 | 11.1 | 8 | 7.6 |
| NO: 125 | | | | | | | | | |
| SEQ ID | 267 | 710 | 95.8 | 61 | 69 | 84 | 8.3 | 8.5 | 8 |
| NO: 126 | | | | | | | | | |
| SEQ ID | 158 | 460 | 94.6 | 60 | 69 | 92 | 32 | 7.1 | 6.7 |
| NO: 127 | | | | | | | | | |
| SEQ ID | 233 | 670 | 96.2 | 59 | 69 | | 23.2 | 7.5 | 7.1 |
| NO: 128 | | | | | | | | | |
| SEQ ID | 167 | 500 | 89.4 | 59 | 69 | 76 | 42.4 | 7.1 | 6.7 |
| NO: 129 | | | | | | | | | |
| SEQ ID | 250 | 710 | 83.4 | 58 | 68 | 76 | 24.6 | 7.5 | 7.1 |
| NO: 130 | | | | | | | | | |
| SEQ ID | 200 | 500 | 91.9 | 59 | 69 | 76 | 16.4 | 7.5 | 7.1 |
| NO: 131 | | | | | | | | | |
| SEQ ID | 283 | 710 | 86 | 59 | 69 | 76 | 11.2 | 8.1 | 7.6 |
| NO: 132 | | | | | | | | | |
| SEQ ID | 183 | 480 | 86.7 | 60 | 69 | 74 | 30.6 | 7.5 | 7.1 |
| NO: 133 | | | | | | | | | |
| SEQ ID | 250 | 690 | 91.4 | 60 | 69 | 74 | 21.6 | 8 | 7.6 |
| NO: 134 | | | | | | | | | |
| SEQ ID | 192 | 520 | 86 | 60 | 69 | 73 | 29.5 | 7.5 | 7.1 |
| NO: 135 | | | | | | | | | |
| SEQ ID | 258 | 730 | 90.2 | 59 | 69 | 32 | 17.6 | 8 | 7.6 |
| NO: 136 | | | | | | | | | |
| SEQ ID | 217 | 520 | 89.2 | 59 | 69 | 76 | 11.4 | 8 | 7.6 |
| NO: 137 | | | | | | | | | |
| SEQ ID | 292 | 730 | 94.8 | 60 | 69 | | 7.5 | 8.6 | 8 |
| NO: 138 | | | | | | | | | |

¹Potency based on transient hRXFP1 assay described above.
²Concentration of NaCl in mM at peak elution from column.
ND—not determined.

TABLE 23
In vitro Characteristics of Relaxin-2 Fusion Protein Analogs.

| | Heparin | | | | | | Calculated |
|---|---|---|---|---|---|---|---|
| | binding | SEC | NanoDSF | | | | isoelectric |
| | [NaCl] | Monomer | Tₒₙₛₑₜ | Tₘ | T_{agg} | Potency¹ | point |
| Sample | (mM)² | (%) | (° C.) | (° C.) | (° C.) | (nM) | (pI) |
| SEQ ID | 275 | 86.8 | 52 | 65 | 51 | 5.6 ± 5.2 | 7.62 |
| NO: 516 | | | | | | (n = 2) | |
| SEQ ID | 217 | 93.5 | 59 | 69 | 73 | 15.5 ± 17 | 7.61 |
| NO: 517 | | | | | | (n = 2) | |
| SEQ ID | 208 | 92.9 | 59 | 69 | 73 | 15.8 ± 14 | 7.16 |
| NO: 518 | | | | | | (n = 2) | |
| SEQ ID | 183 | 93.9 | 59 | 69 | 73 | 38.1 ± 37 | 7.14 |
| NO: 519 | | | | | | (n = 2) | |
| SEQ ID | 258 | 90.4 | 60 | 68 | 62 | 4.9 ± 4.7 | 7.97 |
| NO: 520 | | | | | | (n = 2) | |
| SEQ ID | 242 | 94.6 | 59 | 69 | 73 | 3.3 ± 3.6 | 7.97 |
| NO: 521 | | | | | | (n = 2) | |
| SEQ ID | 242 | 91.9 | 60 | 70 | 73 | 6.5 ± 6.8 | 7.62 |
| NO: 522 | | | | | | (n = 2) | |
| SEQ ID | 217 | 94.9 | 60 | 69 | 73 | 9.3 ± 8.9 | 7.61 |
| NO: 523 | | | | | | (n = 2) | |

¹Potency based on transient hRXFP1 assay described above.
²Concentration of NaCl in mM at peak elution from column.
ND— not determined.

All samples in Tables 21, 22, and 23 are LALA PA LS IgG-RelB-Linker-RelA fusions containing the LALA PA LS IgG (SEQ ID NO: 79 or 83) except for wild-type human relaxin-2 and SEQ ID NO: 497. As shown in Tables 21, 22 and 23, above, there is a correlation between lower pI and lower non-specific binding found through heparin chromatography.

Confirmatory AC-SINS and BVP ELISA assays, and additional cAMP potency assays were performed on a subset of relaxin-2 fusion protein analogs, with results shown in Table 24.

TABLE 24
Additional Potency Assays for Relaxin-2 Fusion Protein Analogs.

| | | | | THP1 | Transient | Transient |
|---|---|---|---|---|---|---|
| | | | | (endogenous | HEK293 | HEK293 |
| | | ACSINS | BVP | RXFP1) CAMP | hRXFP1cAMP | rRXFP1cAMP |
| Sample | pI | (nm) | ELISA | EC50 (nM) (n) | EC50 (nM) (n) | EC50 (nM) (n) |
| Wild-type | ND | ND | ND | 0.2 ± 0.2 (7) | 0.039 ± 0.015 (3) | 0.122 ± 0.07 (3) |
| human | | | | | | |
| relaxin-2 | | | | | | |
| SEQ ID NO: | ND | 28 | 7 | ND | 0.3** | 0.95 ± 0.10 (2) |
| 497 | | | | | | |
| SEQ ID NO: | 9.1 | 14 | 34 | 2.0 ± 1.3 (4) | 0.19 ± 0.15 (2) | 1.15 ± 0.23 (2) |
| 496 | | | | | | |
| SEQ ID NO: | 7 | 0 | 1 | ND | ~150 | ND |
| 367 | | | | | | |
| SEQ ID NO: | 7.9 | 3 | 2 | 49 ± 9 (4) | 10.0 ± 4.27 (3) | 15.1 ± 2.17 (3) |
| 313 | | | | | | |
| SEQ ID NO: | 7.5 | 7 | 1 | 63 ± 3 (3) | 38.2 (1) | 31.2 ± 11.1 (3) |
| 317 | | | | | | |
| SEQ ID NO: | 7.9 | 5 | 3 | 30 ± 20 (3) | 7.46 ± 3.32 (3) | 24.5 ± 18.8 (3) |
| 86 | | | | | | |
| SEQ ID NO: | 7.9 | 6 | 2 | 25 ± 12 (4) | 8.70 ± 4.40 (3) | 10.3 ± 3.45 (3) |
| 87 | | | | | | |
| SEQ ID NO: | 8.8 | 15 | 4 | 4.4 ± 0.9 (2) | 0.6 + 0.5 (3) + | ND |
| 90 | | | | | I:BC | |
| SEQ ID NO: | 7.4 | 8 | 1 | 30 ± 20 (3) | 10.2 ± 5.86 (3) | 12.4 ± 3.07 (3) |
| 91 | | | | | | |
| SEQ ID NO: | 7 | 5 | 2 | 90 ± 10 (2) | 19.1 ± 4.80 (3) | 21.3 ± 9.59 (2) |
| 93 | | | | | | |
| SEQ ID NO: | 7.4 | 6 | 3 | 26 ± 4 (4) | 10.9 ± 5.26 (3) | 13.5 ± 0.98 (2) |
| 95 | | | | | | |
| SEQ ID NO: | 7 | 6 | 1 | 100 ± 40 (2) | 21.5 ± 13.5 (3) | 29.1 ± 5.71 (2) |
| 98 | | | | | | |
| SEQ ID NO: | 7.4 | 11 | 3 | 11 ± 2 (3) | 6.17 ± 2.21 (3) | 6.74 ± 0.73 (2) |
| 104 | | | | | | |
| SEQ ID NO: | 7.4 | 6 | 3 | 80 ± 50 (2) | 5.94 ± 1.32 (3) | 6.74 ± 4.57 (2) |
| 111 | | | | | | |
| SEQ ID NO: | 7.1 | 5 | 2 | 200 ± 100 (2) | 25.0 ± 6.20 (3) | 20.3 ± 1.12 (2) |
| 118 | | | | | | |

ND—not determined.

A subset of relaxin-2 fusion protein analog (SEQ ID NO: 496, SEQ ID NO: 313, SEQ ID NO: 87, SEQ ID NO: 90, SEQ ID NO: 95, and SEQ ID NO: 104) samples was tested under various developability assessments. Under high concentration stress (target concentration of about 100 mg/mL), none of the samples showed loss of protein concentration based on UV. Thermal stress induced increased turbidity/opalescence for all samples tested. Agitation stress had no effect on SEQ ID NO: 313 and SEQ ID NO: 104 samples. Under chemical stress (target concentration of about 5 mg/mL), SEQ ID NO: 496 and SEQ ID NO: 90 samples showed reduction in concentration for all chemical stresses tested, while SEQ ID NO: 313, SEQ ID NO: 87, SEQ ID NO: 95, and SEQ ID NO: 104 samples appeared stable. All samples showed oxidation induced reduction in concentration, with SEQ ID NO: 496 and SEQ ID NO: 90 oxidized samples showing elevated high molecule weight species detected by size exclusion chromatography. In addition, based on non-reducing capillary electrophoresis samples (CE-SDS NR), no significant fragmentation was observed for any stresses in any of the tested samples.

The formation of stress induced post translational modifications (PTMs) was also tested. Stresses included incubation at 40° C. for 4 weeks, room temperature and high pH (Tris buffer pH 8) for 2 weeks, room temperature and low pH (glycine buffer pH 3) for 2 weeks and room temperature oxidative stress (0.02% hydrogen peroxide) for 24 hours. SEQ ID NO: 313 and SEQ ID NO: 87 did not show any stress induced modifications; SEQ ID NO: 90 and SEQ ID NO: 95 showed aspartic acid isomerization in the linker region; SEQ ID NO: 496 showed asparagine deamidation in the linker region; and SEQ ID NO: 95 and SEQ ID NO: 104 showed aspartic acid isomerization in the relaxin sequences.

During manufacturability assessments, color change and aggregation were observed with multiple molecules under certain stress conditions, which is common in oxidized proteins. The color change is typically driven by tryptophan oxidation, which drives a change in absorbance at 320 nm and 365 nm (Ambrogelly (2021) Antibodies 10 (2): 21). In order to probe light sensitivity of relaxin-2 fusion protein analogs SEQ ID NO: 522 and SEQ ID NO: 523, a light box was created using a tabletop 25° C. incubator with a transparent glass door. A photometer was attached to the inside of the incubator, a rectangular LED with an adjustable light intensity switch was placed on the outside of the incubator, and the light intensity was adjusted to 1000 lux, which is similar to the light intensity in standard manufacturing suite rooms. Samples were placed in transparent glass vials with 200 μL glass inserts to allow for maximum surface area for light exposure, while maintaining minimal sample volume requirements. Relaxin-2 fusion protein analogs SEQ ID NO: 522 and SEQ ID NO: 523 were incubated at 25° C., 1000 lux for 7 or 14 days. Samples were also subjected to thermal stress via incubation at 40° C. for 7 or 14 days. After light or heat exposure, samples were evaluated using size exclusion high-performance liquid chromatography (SEC) to assess aggregate formation and CE-SDS to assess purity. As shown in Table 25, under these conditions, SEQ ID NO: 522 and SEQ ID NO: 523 are both resistant to thermal and light stress as assessed by aggregate formation and purity.

TABLE 25
Light and Heat Stress Stability of Relaxin-2
Fusion Protein Analogs.

| | | | | CE-SDS | |
|---|---|---|---|---|---|
| | | SEC | | Reduced | Non- |
| | | Monomer | HMW | purity | reduced |
| Sample | Condition | (%) | (%) | (%) | purity (%) |
| SEQ ID | Control | 96.2 | 3.8 | 99.5 | 90.4 |
| NO: 522 | (unstressed) | | | | |
| | Light | 95.5 | 4.5 | 99.5 | 90.4 |
| | (7 days) | | | | |
| | Light | 95.7 | 4.3 | 99.5 | 90.6 |
| | (14 days) | | | | |
| | 40° C. | 95.6 | 4.4 | 99.5 | 90.1 |
| | (7 days) | | | | |
| | 40° C. | 95.2 | 4.8 | 99.5 | 89.9 |
| | (14 days) | | | | |
| SEQ ID | Control | 90.6 | 9.4 | 99.5 | 94.7 |
| NO: 523 | (unstressed) | | | | |
| | Light | 90.1 | 9.9 | 99.5 | 94.4 |
| | (7 days) | | | | |
| | Light | 90.5 | 9.5 | 99.5 | 94.6 |
| | (14 days) | | | | |
| | 40° C. | 90.4 | 9.6 | 99.5 | 94.7 |
| | (7 days) | | | | |
| | 40° C. | 90.3 | 9.7 | 99.5 | 94.9 |
| | (14 days) | | | | |

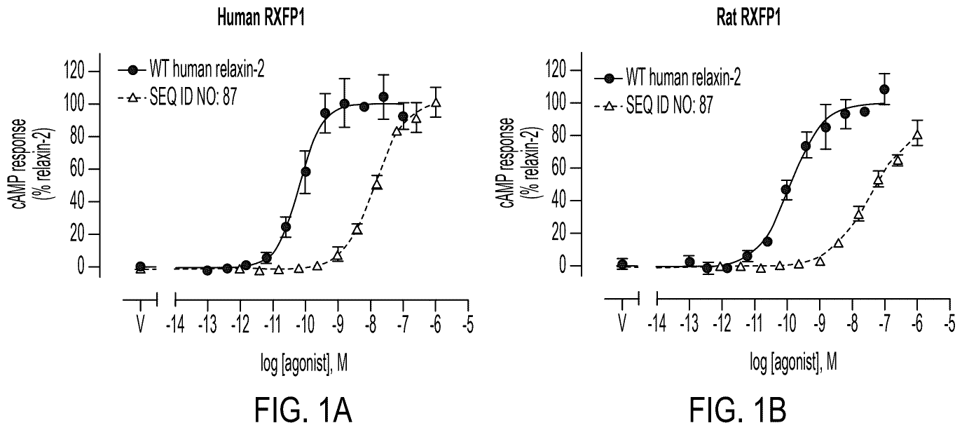

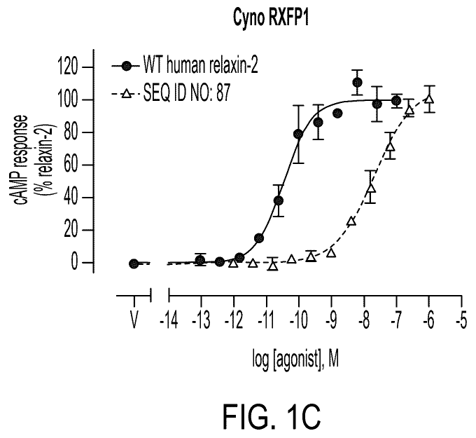

A separate study was performed to investigate the in vitro potency of SEQ ID NO: 87 as compared to wild-type (WT) human relaxin-2, using mammalian cell systems that either transiently, stably, or endogenously expressed human RXFP1 or orthologs from cynomolgus monkey and rat. Table 26 shows a summary of cAMP response induced by SEQ ID NO: 87 and wild-type human relaxin-2 on HEK293 cells transiently expressing human, monkey, and rat RXFP1. As shown in Table 26 and FIGS. 1A-1C, the average EC50 for SEQ ID NO: 87 for human (FIG. 1A), rat (FIG. 1B), and monkey (FIG. 1C) RXFP1 was 10±4 nM, 10±9 nM, and 30±20 nM, respectively.

TABLE 26
Summary of cAMP Response in Cells Transiently Expressing RXFP1.

| | Wild-type human relaxin-2 EC₅₀ | | SEQ ID NO: 87 EC₅₀ | |
|---|---|---|---|---|
| | nM | ng/mL | nM | ng/mL |
| | Mean ± SD (n) | Mean ± SD (n) | Mean ± SD (n) | Mean ± SD (n) |
| Human RXFP1 in | 0.05 ± 0.02 (6) | 0.3 ± 0.1 (6) | 10 ± 4 (6) | 800 ± 300 (6) |
| HEK293 | | | | |
| Rat RXFP1 in | 0.1 ± 0.02 (3) | 0.6 ± 0.1 (3) | 30 ± 20 (3) | 2000 ± 1000 (3) |
| HEK293 | | | | |
| Cynomolgus | 0.04 ± 0.01 (3) | 0.2 ± 0.1 (3) | 10 ± 9 (3) | 800 ± 600 (3) |
| monkey RXFP1 | | | | |
| in HEK293 | | | | |

To test the selectivity of SEQ ID NO: 87, CHO-K1 cells stably expressing human RXFP1 or human RXFP2 were used. As shown in Table 27, the EC₅₀ for SEQ ID NO: 87 for human RXFP1 was 40±20 nM, and for human RXFP2, was ˜100-fold less potent (EC₅₀ of ≥2000 nM), demonstrating that SEQ ID NO: 87 is selective for RXFP1.

TABLE 27
Summary of cAMP Response in Cells Stably or Endogenously Expressing
RXFP1.

| | Wild-type human relaxin-2 EC₅₀ | | SEQ ID NO: 87 EC₅₀ | |
|---|---|---|---|---|
| | nM | ng/mL | nM | ng/mL |
| | Mean ± SD (n) | Mean ± SD (n) | Mean ± SD (n) | Mean ± SD (n) |
| Human RXFP1 | 0.2 ± 0.1 (4) | 1 ± 0.6 (4) | 40 ± 20 (4) | 2000 ± 1000 (4) |
| (stable) in | | | | |
| CHO-K1 | | | | |
| Human RXFP2 | 10 ± 10 (4) | 90 ± 60 (4) | >2000 (4) | >10⁶ (4) |
| (stable) in | | | | |
| CHO-K1 | | | | |
| THP-1 | 0.07 ± 0.03 (5) | 0.4 ± 0.2 (5) | 10 ± 6 (5) | 900 ± 400 (5) |
| (endogenous | | | | |
| RXFP1) | | | | |

In some cases, transient or stable ectopic expression of proteins in cells can result in over-expression of targets, which may impact the potency and efficacy of test articles. To address this, the potency of SEQ ID NO: 87 was tested in the human leukemia monocytic cell line, THP-1, which endogenously expressed RXFP1. As shown in Table 27, the EC50 for SEQ ID NO: 87 was found to be 10±6 nM, consistent with what was found in the in vitro RXFP1 potency assays described above.

### Example 5. Pharmacokinetic (PK) and Pharmacodynamic (PD) Properties of Relaxin-2 Fusion Protein Analogs

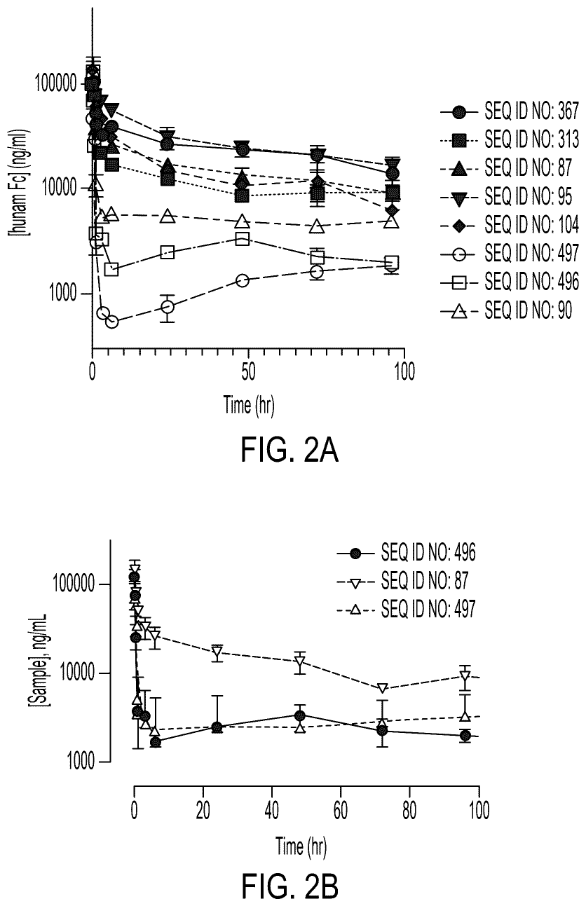

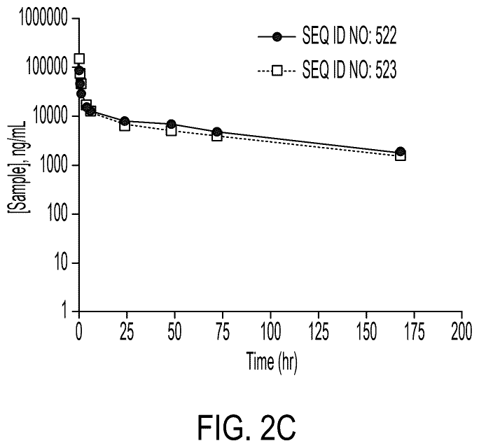

Pharmacokinetic (PK) values were determined by measuring the concentration of a relaxin-2 fusion protein analog in the plasma of rats following a 5 mg/kg intravenous (IV) injection of the respective protein analog over time. PK values were determined for a subset of relaxin-2 fusion protein analog (SEQ ID NO: 496, SEQ ID NO: 497, SEQ ID NO: 367, SEQ ID NO: 313, SEQ ID NO: 87, SEQ ID NO: 95, SEQ ID NO: 90, and SEQ ID NO: 104) samples described herein (FIG. 2A). Rat PK parameters are shown in Table 28, below.

TABLE 28
Summary of PK Parameters.

| | SEQ ID | SEQ ID | SEQ ID | SEQ ID | SEQ ID | SEQ ID | SEQ ID | SEQ ID |
|---|---|---|---|---|---|---|---|---|
| | NO: 496 | NO: 313 | NO: 87 | NO: 90 | NO: 95 | NO: 104 | NO: 367 | NO: 497 |
| Parameter | rat (mL/h or mL) | | | | | | | |
| Clearance CL | 2.76 | 0.69 | 0.52 | 0.29 | 0.32 | 0.52 | 0.356 | ND |
| Central | 6.73 | 11.5 | 11.28 | 13.46 | 9.45 | 16.63 | 15.798 | ND |
| Volume 1 | | | | | | | | |
| Peripheral | 28.24 | 16.8 | 8.42 | 32.18 | 1.12 | 2.28 | 12.014 | ND |
| Clearance | | | | | | | | |
| Peripheral | 413.8 | 56.3 | 47.82 | 249.95 | 17.41 | 50.22 | 21.655 | ND |
| Volume | | | | | | | | |
| Peripheral | ND | ND | 0.13 | ND | 3.95 | ND | ND | ND |
| Clearance 2 | | | | | | | | |
| Peripheral | ND | ND | 0.11 | ND | 6.07 | ND | ND | ND |
| Volume 2 | | | | | | | | |

ND—not determined.

FIG. 2B shows a separate experiment with PK values determined using the same methods described for FIG. 2A, for a subset of relaxin-2 fusion protein analog (SEQ ID NO: 87, SEQ ID NO: 496, and SEQ ID NO: 497) samples described herein.

FIG. 2C shows a separate experiment with PK values determined using the same methods described for FIG. 2A, for a subset of relaxin-2 fusion protein analog (SEQ ID NO: 522 and SEQ ID NO: 523) samples described herein.

### Example 6. Hemodynamics and Renal Blood Flow Effects of Relaxin-2 Fusion Protein Analogs

The high isoelectric point (pI) of relaxin and related molecules presents significant pharmacokinetic (PK) and biophysical challenges, reflected in the rapid decline observed in serum concentration for these molecules in the earliest timepoints of the PK curve. Without being bound by any theory, this high clearance phenomenon has been attributed to non-specific binding of high pI molecules to negatively charged heparin proteoglycans in the vasculature and in tissues. These issues were resolved through structure-guided engineering of the relaxin-2 fusion protein analogs to reduce their pI, as shown in Examples 1-5.

To assess the impact of the changes to lower pI, the PK and pharmacodynamic (PD) effects of the relaxin-2 fusion protein analogs were measured in rats. One of the most readily quantifiable activities of relaxin is to produce an observable increase in renal arterial blood flow (RABF) shortly following administration. This is a PD effect that has been shown to be observable in both rats and human patients with administration of serelaxin and can be modeled to establish a PK/PD relationship for test compounds.

Naïve male Sprague-Dawley rats (weight range: 0.308-0.399 kg) were anesthetized via 5% isoflurane driven by 100% oxygen in an induction chamber. Once consciousness was lost, animals were removed from the chamber and an endotracheal tube was inserted for mechanical positive-pressure ventilation. The ventilator was connected to a vaporizer that delivered approximately 1-2% isoflurane driven by 100% oxygen for the duration of the experiment. Anesthetic depth was assessed prior to surgery and approximately every 15 minutes during the experimental procedure. Animals were maintained at approximately 37±0.5° C. on a heating pad, and body temperature was monitored throughout the protocol with a rectal temperature probe.

A Millar pressure catheter was placed in the right carotid artery to measure systolic arterial pressure (SAP), diastolic arterial pressure (DAP) and heart rate (HR). Mean arterial pressure (MAP) was calculated. A small incision was made along the linea alba to access the abdominal cavity. The left renal artery was dissected and a doppler flow probe was placed around the artery. Renal artery blood flow (RABF) was continuously monitored throughout the experimental period, and renal vascular resistance (RVR=MAP/RABF) was calculated.

Following an approximately 10-15-minute equilibration period, baseline (BL) measurements were collected for 15 minutes. Individual animals were deemed acceptable for use in the study based on health status, body weight, hemodynamic parameters and renal blood flow. Following BL measurements, rats received a bolus intravenous (IV) administration (Dose 1) of either vehicle (10 mM histidine, 50 mM NaCl, 6.5% trehalose in deionized water pH 6.0) or a relaxin-2 fusion protein analog (test compound). Immediately following the bolus IV dose, rats in each group received a maintenance dose via IV infusion (Dose 2) of the respective test compound over a 180-minute dosing period.

Blood samples were collected prior to Dose 1 (before the end of BL) and at 5 minutes, 1 hour, 2 hours and 3 hours after the start of Dose 2. Samples were obtained from a jugular vein cannula into a K2EDTA tube. Tubes were stored on wet ice until centrifugation in a refrigerated centrifuge. The resultant plasma was frozen on dry ice and stored at −80° C. At the conclusion of the study, rats were euthanized by exsanguination.

Mean values taken from 15-minute blocks during BL and from the 180-minute dose period were used for analysis. Values from each individual animal were pooled to determine an average for each variable for each group (if applicable). Average percent change from baseline values were determined for each variable. The term “dosing period” will describe the period during bolus and maintenance dose infusion (180 minutes) and will be used for the remainder of this report.

In one experiment, the effect on renal blood flow in rats of relaxin-2 fusion protein analogs SEQ ID NO: 313 and SEQ ID NO: 87 were compared to prior fusion protein SEQ ID NO: 496. Table 29 below shows the potency of relaxin-2 fusion protein analogs in recombinant human and rat RXFP1 assays (as described in Example 4).

TABLE 29
Fusion Protein Analogs in Human and Rat RXFP1 Potency Assays

| | HEK293 cells expressed RXFP1 cAMP | |
|---|---|---|
| | assay (EC50 (nM) ± SD (n)) | |
| | Human RXFP1 | Rat RXFP1 |
| Wild-type human | 0.04 ± 0.01 (4) | 0.11 ± 0.06 (4) |
| relaxin-2 | | |
| SEQ ID NO: 496 | 0.2 ± 0.2 (3) | 1.2 ± 0.2 (3) |
| SEQ ID NO: 313 | 10 ± 4 (4) | 17 ± 5 (4) |
| SEQ ID NO: 87 | 7 ± 4 (4) | 10 ± 3 (4) |
| SEQ ID NO: 90 | 0.9 ± 0.5 (2) | 3 (1) |

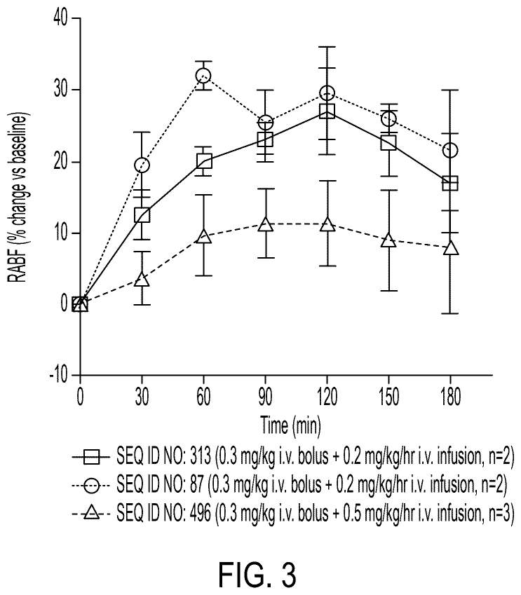

As shown in FIG. 3, administration of SEQ ID NO: 313 and SEQ ID NO: 87 at a bolus intravenous dose of 0.3 mg/kg and intravenous infusion of 0.2 mg/kg/hr caused a greater increase in rat RABF than administration of prior fusion protein SEQ ID NO: 496 at a bolus intravenous dose of 0.3 mg/kg and intravenous infusion of 0.5 mg/kg/hr. As such, despite the reduced in vitro potency observed for SEQ ID NO: 313 and SEQ ID NO: 87, these fusion proteins demonstrate greater increase in rat RABF.

Additional experiments were performed to further evaluate the effect of fusion protein analog SEQ ID NO: 87 and SEQ ID NO: 497 on renal blood flow. In these experiments, the average potency of relaxin-2 fusion protein analogs in recombinant human and rat RXFP1 assays are shown in Table 30, below.

TABLE 30
Fusion Protein Analogs in Human and Rat RXFP1 Potency Assays

| | EC50 for cAMP generation in | |
|---|---|---|
| | recombinant RXFP1 assay (nM) | |
| | Human RXFP1 | Rat RXFP1 |
| Wild-type human | 0.04 | 0.1 |
| relaxin-2 | | |
| SEQ ID NO: 496 | 0.2 | 1 |
| SEQ ID NO: 87 | 10 | 35 |
| SEQ ID NO: 497 | 0.3 | 1 |

SEQ ID NO: 87 was infused by cannulated femoral vein using a syringe pump. The dose was infused intravenously (1 ml/kg) as a bolus followed by continuous infusion of PBS at the rate of 0.5 mL/kh/hr to maintain the circulating fluid volume.

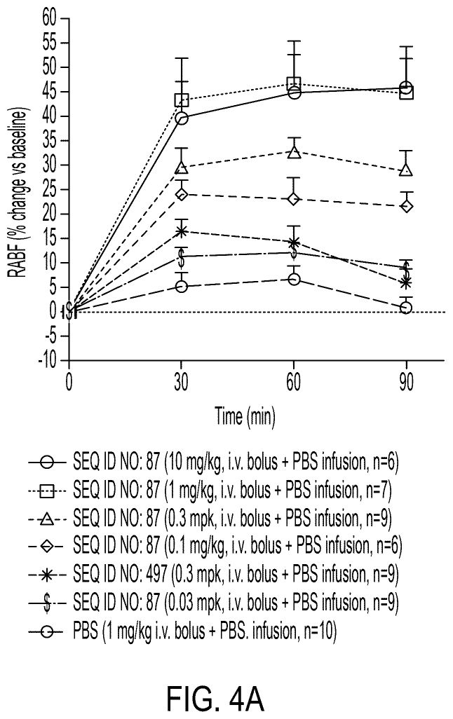

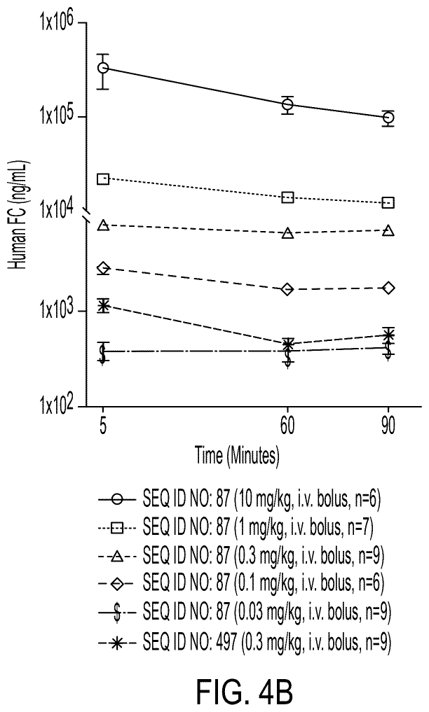

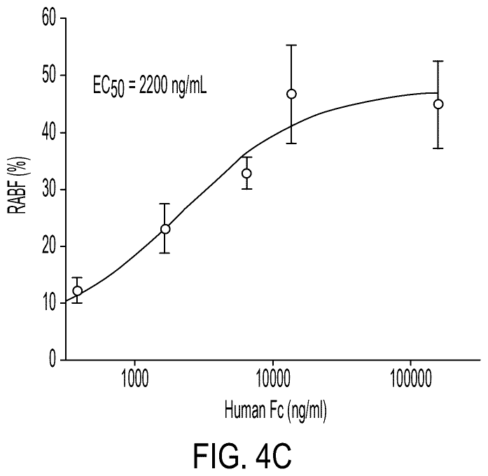

As shown in FIG. 4B, the measured serum concentration (using human Fc levels as proxy) for 0.3 mg/kg SEQ ID NO: 497 was ˜10 fold lower than that of 0.3 mg/kg SEQ ID NO: 87. Given the fact that the in vitro potency of SEQ ID NO: 497 in the rat RXFP1 signaling assay was found to be more than 30-fold higher than the in vitro potency of SEQ ID NO: 87 (see, Table 30), one would expect that the two molecules would give at least comparable increases in RABF when administered at the same dose. Instead, the efficacy of SEQ ID NO: 497 was comparable to a dose of SEQ ID NO: 87 that was 10-fold lower (0.03 mg/kg) (FIG. 4A), implying that the efficacy of SEQ ID NO: 87 was more than 10-fold higher than expected based on PK and in vitro potency data. The plasma concentration for 0.03 mg/kg SEQ ID NO: 87 was nearly identical to that for 0.3 mg/kg SEQ ID NO: 497.

The PBS data in FIG. 4A show that there is a small effect on renal blood flow from the volume expansion from the intravenous bolus that returned to baseline by 90 minutes. Using the 90-minute timepoint to fit the dose response for SEQ ID NO: 87 from 0.03 mg/kg to 10 mg/kg, the EC50 for SEQ ID NO: 87 in the rat in vivo was estimated to be ˜2200 ng/ml (FIG. 4C) which corresponds to about 34 nM, consistent with the average EC50 for SEQ ID NO: 87 signaling as shown in Table 30.

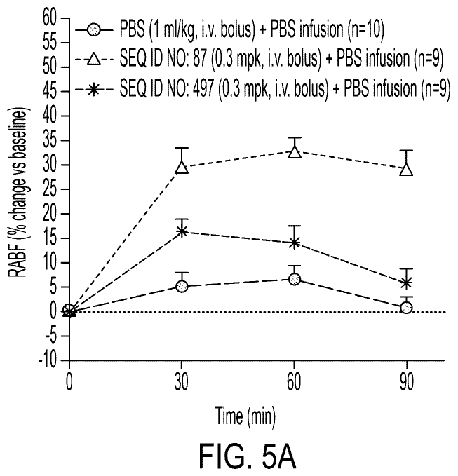

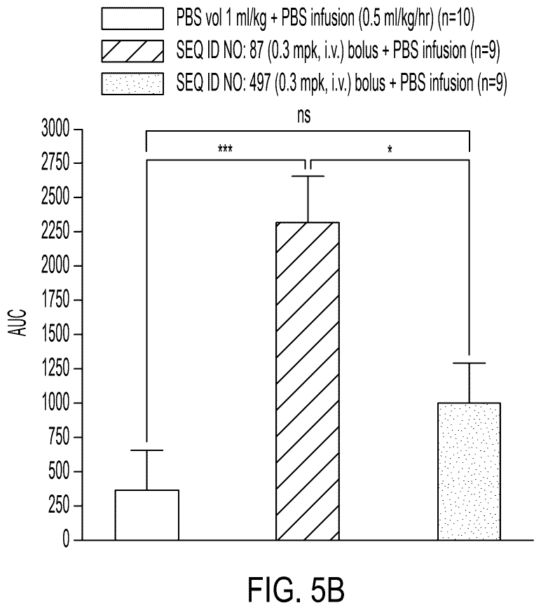

Additionally, as shown in FIGS. 5A and 5B, a low dose of SEQ ID NO: 87 increased and maintained RABF more than SEQ ID NO: 497. At 0.3 mg/kg, infusion of SEQ ID NO: 87 resulted in increased RABF by approximately 25% over baseline, which was maintained over the 90-minute experimental time course. At 0.3 mg/kg, infusion of SEQ ID NO: 497 resulted in increased RABF by approximately 15% over baseline, and RABF levels dropped close to baseline within 90 minutes post-infusion (FIG. 5A). Quantification of the area under the curve (AUC) showed that infusion of SEQ ID NO: 87 significantly increased RABF about 2-fold more than infusion of SEQ ID NO: 497 (FIG. 5B).

Without being bound by any theory, it is hypothesized that the enhanced effect of SEQ ID NO: 87 is due to an increase in distribution of SEQ ID NO: 87 to target tissues well above levels expected from plasma levels because of reduced heparin binding, as well as decreased non-specific cellular uptake, blood clearance and potentially higher bioavailability of SEQ ID NO: 87 compared to the more highly charged SEQ ID NO: 497 molecule.

### Example 7. Therapeutic Effect of a Relaxin-2 Fusion Protein Analog in a Rat Pulmonary Arterial Hypertension Model

Pulmonary arterial hypertension (PAH) is characterized by a progressive pulmonary vascular remodeling of the distal precapillary arteries that causes a significant increase of the right ventricle (RV) load, ultimately leading to right heart failure and premature death. Using monocrotaline (MCT) rat model as an in vivo PAH model, rats reproducibly develop pulmonary hypertension with a mean pulmonary pressure of ˜40 mmHg approximately 4 weeks after single MCT administration. MCT is an 11-membered macrocyclic pyrrolizidine alkaloid derived from the seeds of the Crotalaria spectabilis plant. The MCT alkaloid is activated to the reactive pyrrole metabolite dehydromonocrotaline (MCTP) in the liver, a reaction that is highly dependent on cytochrome P-450 (CYP3A4). When administered, MCT induces a syndrome characterized, among other manifestations, by pulmonary hypertension (PH), pulmonary mononuclear vasculitis, and right-ventricular hypertrophy through injury to pulmonary endothelial cells. The therapeutic potential of the SEQ ID NO: 87 relaxin-2 fusion protein analog was evaluated in a rat model of MCT-induced PAH.

On Study Day 1, naïve young (200-240 g) male Sprague Dawley rats in groups 1-5 received a 60 mg/kg dose (1 mL/kg s.c. in 100% DMSO) of MCT. Rats in Group 1 and 4 received anti-mouse CD20 antibody (20 mg/kg, i.p.) on days 8, 9, 10, and 17. Rats in Group 1 and 2 received SEQ ID NO: 87 (10 mg/kg, i.v.) on days 7, 10, 14, 17, 21 and 24. Beginning on study day 8, rats in Group 5 were dosed orally twice daily with sildenafil (positive control, 30 mg/kg, p.o., BID) with the last dose administered on study day 28. Rats in Group 6 were administered DMSO as control. 12 rats were tested in each of Groups 1 to 4, and 10 rats were tested in each of Group 5 and 6.

Cage side observations were done once daily for general health and appearance, mortality and signs of pain or distress. Body weights were recorded pre-dose on study day 0, and weekly throughout the study and on the day of the terminal procedure. On Study Day 28, animals were anesthetized with urethane (1.25 g/kg, i.p.). Blood samples were collected from the Retro-Orbital plexus for PK and biomarker analysis (e.g., N-terminal (NT)-pro hormone BNP (NT-proBNP)). Serum NT pro-BNP was analyzed using Rat NT-proBNP Assay Kit-Meso Scale Discovery, MD, USA (catalog no. K153JKD). Rats were ventilated using RoVent® Jr. Small Animal Ventilator from Kent Scientific Corporation. The mean right ventricular pressure and mean pulmonary artery pressure were measured via a 1.6 French solid-state catheter purchased from Transonic Inc. using an open chest approach. Data were recorded and analyzed using SP200 Pressure System and LabChart software (ADI instruments). The animals were then humanely euthanized via exsanguination while under deep anesthesia. At necropsy, the heart and lungs were collected from each animal for weighing. After weighing, both the right ventricle and lung samples, were fixed in formalin and then stored at room temperature. The brain was also collected and weighed to normalize with the rat body weight. Immunohistochemistry analysis was performed on the formalin fixed lungs and ventricles.

An assay was developed to quantify the presence of SEQ ID NO: 87 in mouse serum samples, coming from both serum and plasma. The assay utilizes a quantitative sandwich enzyme immunoassay, with an affinity-purified polyclonal antibody specific for Human Fc coated to a 96-well plate. Samples containing molecules with Human Fc were added to wells, incubated, and washed before the addition of an enzyme-linked polyclonal antibody specific for Human Fc. After incubation with the enzyme-linked antibody, wells were washed, and the enzyme-substrate was added and allowed to develop before quenching with acid. After quenching with an acid plate, read the plate at 450 and 570 nm within 30 minutes of color change. Values from each animal were pooled to determine an average of each variable for each group (if applicable). The mean right ventricular pressure (mRVP) and mean pulmonary artery pressure (mPAP) were recorded, and Right Ventricular Systolic Pressure (RVSP) was analyzed.

SEQ ID NO: 87 was administered in the MCT-induced PAH model at 10 mg/kg, i.v. twice weekly for 3 weeks with therapeutic dosing initiated one week after MCT injection. To mitigate efficacy reducing anti-drug antibody (ADA) generation to the test article, the study was performed with and without B-cell depletion by injecting anti-mouse CD20 antibody to rats. Cardiac hemodynamics including right ventricular systolic pressure (RVSP) and mean pulmonary arterial pressure (mPAP) were measured using an open chest approach. The Fulton Index, the ratio of right ventricular weight to the weight of the left ventricle and septum, was measured at week 4. Blood samples for serum NT pro-BNP, PK and ADA analysis were collected.

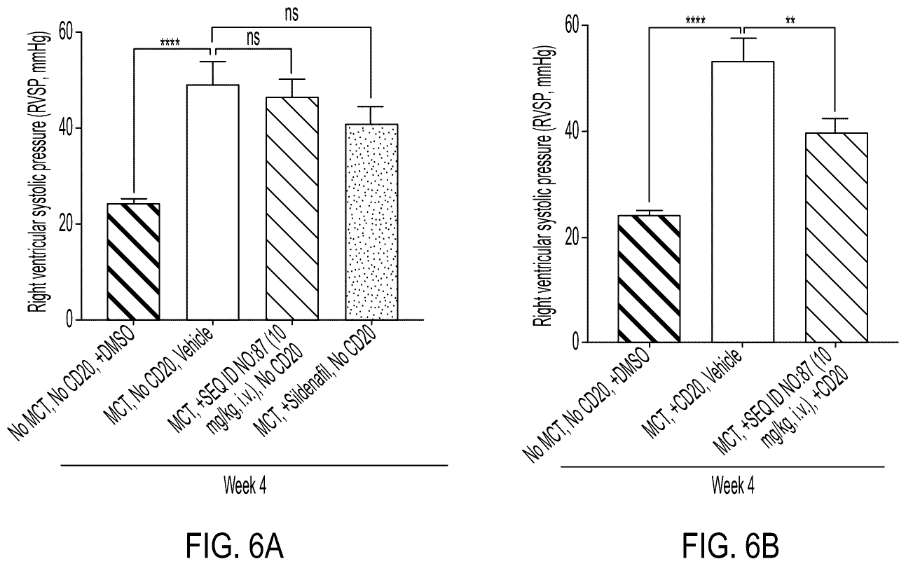

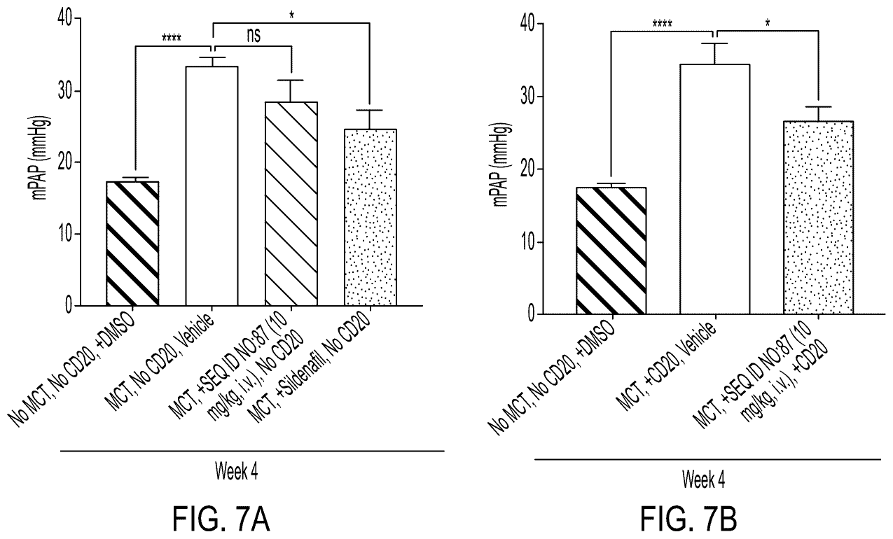

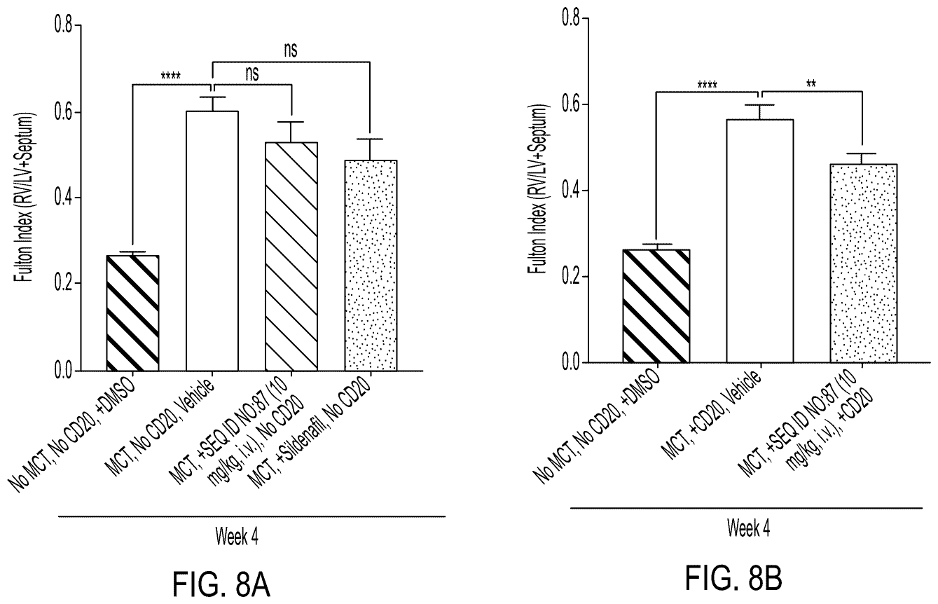

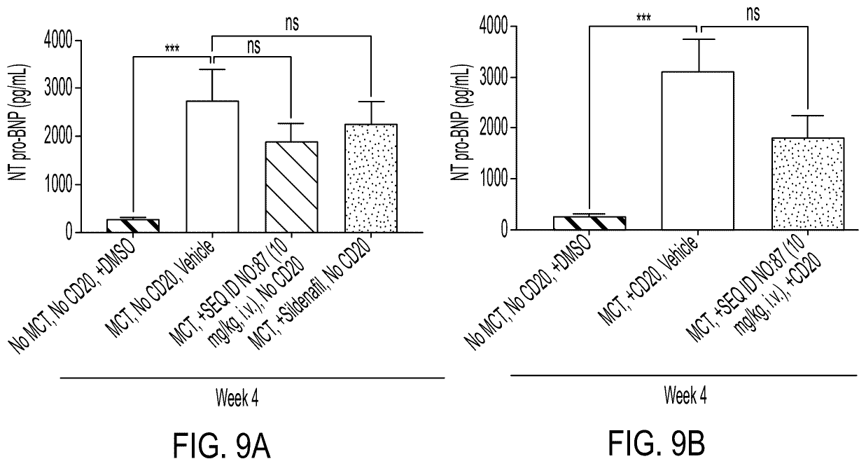

MCT treatment of rats significantly increased RVSP (FIGS. 6A and 6B), mPAP (FIGS. 7A and 7B), Fulton Index value (FIGS. 8A and 8B), and NT-pro-BNP (FIGS. 9A and 9B) while decreasing survival compared to the naïve group (no MCT) over a 4-week period. As shown, rats treated with SEQ ID NO: 87 demonstrated significant improvements in RVSP (FIG. 6B), mPAP (FIG. 7B), and Fulton Index value (FIG. 8B) in rats where B cells were depleted. In addition, there was a clear survival benefit following treatment with SEQ ID NO: 87, with treated rats being the only group showing 100% survival.

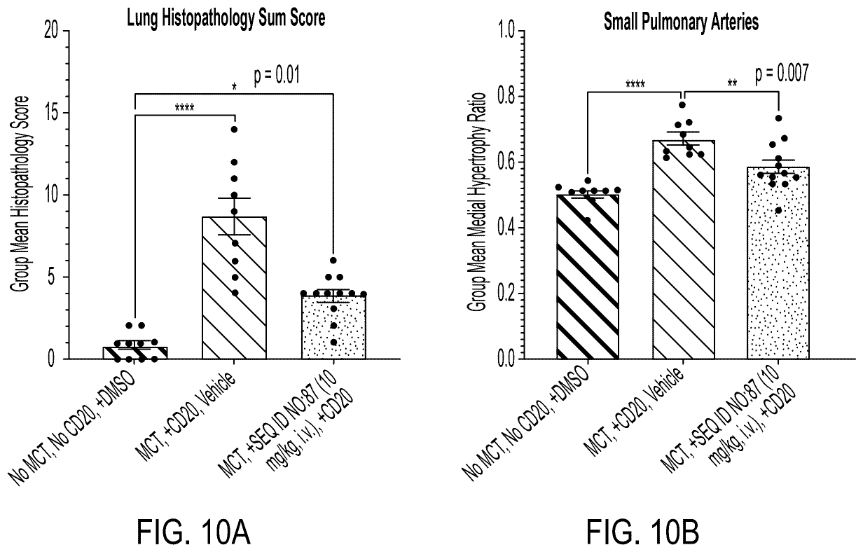

In addition, histopathology analysis revealed that SEQ ID NO: 87 significantly improved lung inflammation and reduced pulmonary arterial muscularization (FIGS. 10A and 10B, respectively). To assess lung inflammation, histopathology analysis was performed on tissues stained using hematoxylin and eosin. The lung histopathology sum score was calculated as the sum scores of parameters analyzed, including pulmonary arterial hypertrophy, vasculitis/necrosis, alveolar histiocytosis, perivascular and interstitial inflammation, hemorrhage, and fibrin deposition. Significant improvement was observed in the overall lung histopathology sum score as well as in the degree of vasculitis/necrosis, hemorrhage, and fibrin deposition in the lung tissues of rats, compared to rats that received vehicle control. To assess pulmonary arterial muscularization, histopathology analysis was performed using tissues stained with an anti-alpha smooth muscle actin (SMA) antibody to quantify muscularization, and Verhoeff stain to stain internal elastic laminae of arterioles. Thickening of the arterial wall, primarily by smooth muscle hypertrophy, was seen at all levels of the pulmonary arterial tree in animals exposed to MCT. This was associated with an increased arterial medial hypertrophy ratio across five vessel sizes quantified. Medial thickening relative to external diameter was best developed in the smallest caliber arteries and arterioles with less dramatic medial hypertrophy seen in the intermediate and larger caliber arteries. Lungs from rats treated with SEQ ID NO: 87 had lower arteriolar, small, and intermediate artery medial hypertrophy ratios than untreated animals. For small caliber arteries, rats treated with SEQ ID NO: 87 had significantly lower ratios than untreated animals.

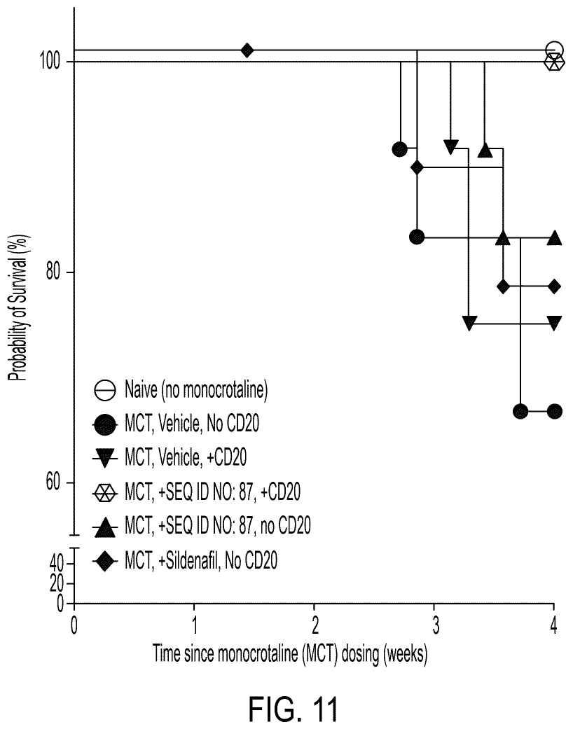

No mortality was observed in B cell depleted rats treated with SEQ ID NO: 87 compared to the vehicle and sildenafil treated groups (FIG. 11).

### Example 8. Anti-Fibrotic Effect of Relaxin-2 Fusion Protein Analogs in a Mouse Model of Kidney Fibrosis

This Example describes evaluation of the anti-fibrotic effect of relaxin-2 fusion protein analogs SEQ ID NO: 496, SEQ ID NO: 313, and SEQ ID NO: 87 in a mouse unilateral ureteral obstruction (UUO) model of kidney fibrosis. The UUO model induces renal fibrosis, where the primary feature of UUO is tubular injury because of obstructed urine flow. Furthermore, experimental UUO in rodents is believed to mimic human chronic obstructive nephropathy in an accelerated manner. Renal fibrosis is the common pathway for most forms of progressive renal disease. Because removal of the obstruction is generally not sufficient to reverse fibrosis, model animals may benefit from an accompanying treatment. UUO is a model widely used to study obstructive nephropathy.

An initial study evaluated the effect of SEQ ID NO: 496 and SEQ ID NO: 313. Sixty-five male C57BL/6 mice were used for the study (8-10 weeks of age at initiation). UUO was induced on Day 0 by ligation of a ureter of the left kidney, while the contra-lateral kidney served as a control. The UUO surgery was performed according to a standard procedure under deep anesthesia. Briefly, the kidney and ureter were exposed through midline abdominal incision after removal of hair from abdominal area. The left ureter was completely obstructed with two ligations. The first suture was placed 1 mm below the kidney and the other suture was placed 1 mm below the renal pelvis with silk or prolene suture. The wound was closed with 2 to 3 staples. The animal was returned to its cage and monitored until it started to move. On the day of surgical procedure and for 72 hours post-surgery, animals were provided with pain medication Buprenorphine. A control group (n=5) underwent sham surgery). UUO mouse groups were treated with 10 mg/kg SEQ ID NO: 496 (n=10), 20 mg/kg SEQ ID NO: 496 (n=10), 10 mg/kg SEQ ID NO: 313 (n=10), or 20 mg/kg SEQ ID NO: 313 (n=10). The treatment groups received intravenous injections 2 days before surgery (Day-2) and on Day 2 and Day 5 post-surgery. A positive control group (n=10) received enalapril, an ACE inhibitor medication used to treat high blood pressure, diabetic kidney disease, and heart failure, which was provided through drinking water at 200 mg/L beginning on Day-2 and continued through study termination. A negative control group (n=10) was treated with vehicle (PBS). The study was terminated on Day 7 post-surgery, and fibrosis symptoms as well as in-life parameters were assessed.

No adverse clinical symptoms were developed in any of the animals post-UUO surgery. The animals tolerated treatment with SEQ ID NO: 496, SEQ ID NO: 313, and enalapril well, and 100% survival was recorded. On Day 7 post-surgery, left kidneys were harvested, weighed, and fixed for histology.

All groups with surgically induced UUO showed reduced body weight relative to the sham surgery group. At Day 4 post-surgery, body weights began to trend toward recovery for all UUO groups. Additionally, all UUO groups showed a significant increase in kidney weight relative to the sham surgery group, and there was no statistically significant change in kidney weight across the treatment or control UUO groups.

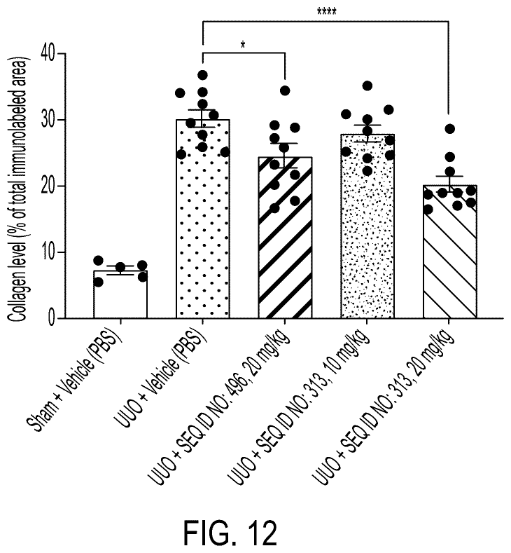

Histological analysis of kidney sections fixed on Day 7 post-surgery showed a significant increase in collagen deposition in renal parenchyma in all UUO groups relative to the sham surgery group (FIG. 12). Animals treated with SEQ ID NO: 496 and SEQ ID NO: 313 at 20 mg/kg showed significantly reduced collagen deposition relative to the vehicle-treated control group.

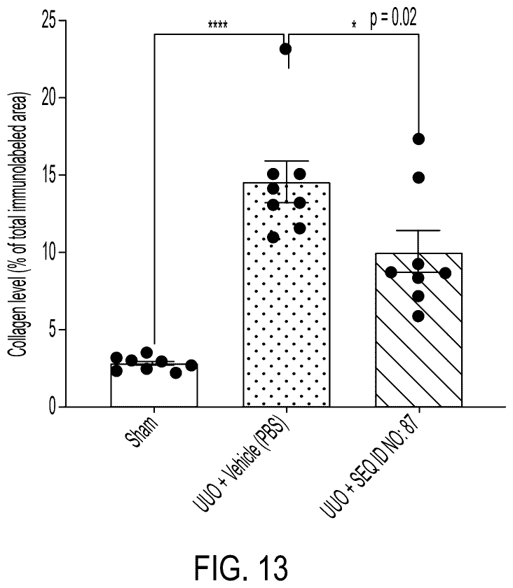

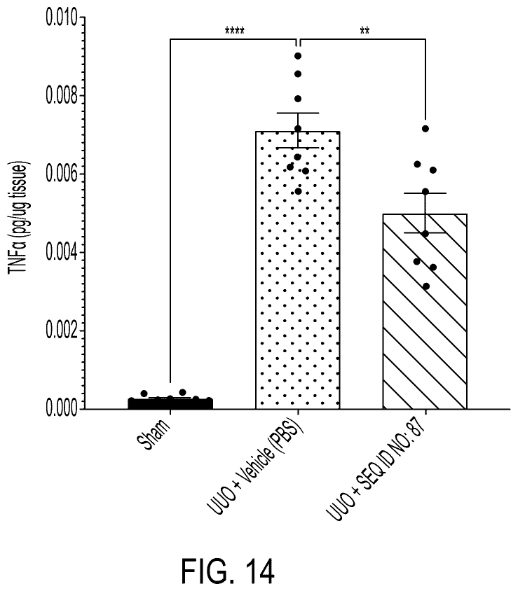

The anti-fibrotic effect of SEQ ID NO: 87 was observed following a similar procedure. Mice treated with SEQ ID NO: 87 showed a significant decrease in collagen deposition following UUO induction relative to vehicle-treated mice (FIG. 13). Tissue TNFα levels were evaluated via electrochemiluminescence, and mice treated with SEQ ID NO: 87 also showed a significant decrease in TNFα levels relative to vehicle-treated mice (FIG. 14). Reduction of IL-1β and IL-6 in fibrotic kidney was also observed in the SEQ ID NO: 87-treated mice relative to vehicle treated mice.

### Example 9. Effect of Relaxin-2 Fusion Protein Analogs on Isoproterenol-Induced Cardiac Hypertrophy and Fibrosis

This Example describes evaluation of the effect of relaxin-2 fusion protein analog SEQ ID NO: 87 on cardiac hypertrophy and fibrosis following isoproterenol challenge. Isoproterenol is a drug that increases heart rate and heart muscle contractions and can lead to cardiac hypertrophy and increased fibrosis.

All animals were treated and cared for in accordance with the Guide for the Care and Use of Laboratory Animals (National Institutes of Health, revised 2011), and protocols were approved by an Institutional Animal Care and Use Committee. Male C57BL/6J mice were obtained at 10-11 weeks of age with body weight ranging between 25 grams and 30 grams. The animals were housed in the vivarium on a conventional 12 hr light/dark cycle. After equilibration housing for 1 week, micro-osmotic minipump implantation was performed on mice. Briefly, mice were anesthetized using 1% to 3% isoflurane given by inhalation through a vaporizer. Osmotic minipumps with a flow rate of 0.25 μl/h were surgically implanted subcutaneously into the subscapular space of mice. Each pump delivered a constant dose (0.25 μl/h) of infused drug (isoproterenol in PBS containing 0.002% ascorbic acid at 15 mg/kg per day) or vehicle (PBS with 0.002% ascorbic acid) for 2 weeks. Postoperative analgesia was achieved with a single dose of meloxicam SR (2.5 mg/kg) during minipump implantation and another single dose of meloxicam SR (2.5 mg/kg) one day after the surgery. Mice were treated with either vehicle or 10 mg/kg of SEQ ID NO: 87 one day before minipump implantation. Mice were then dosed with relaxin-2 fusion protein analog SEQ ID NO: 87 bi-weekly for the duration of the study (total of 14 days). To reduce anti-drug activity, mice were injected intraperitoneally with 20 mg/kg of anti-mouse CD20 to deplete all B cells one day post minipump implantation.

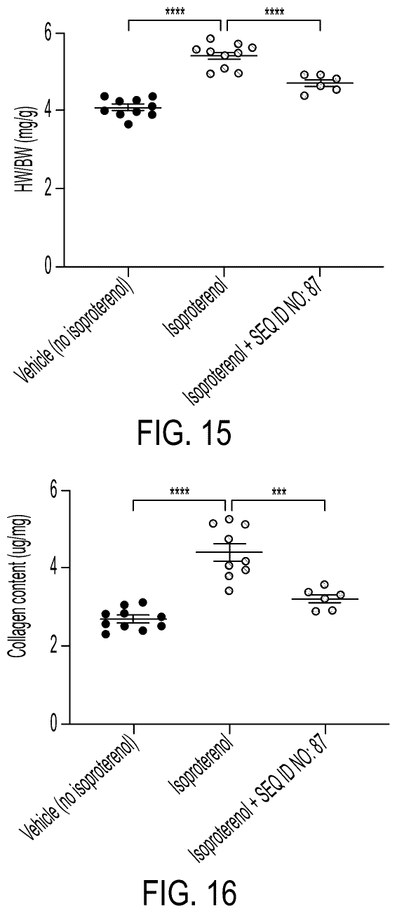

After 14 days of incubation, animals were euthanized via CO₂ inhalation followed by cervical dislocation. The body weights were measured. Whole hearts were explanted, washed in PBS, dried with paper towel, and weighed. The fresh heart tissues were immediately snap frozen in liquid nitrogen and stored for further collagen content analysis. Tibia was obtained by blunt limb dissection and tibia length was measurement using a digital caliper. Heart weights (HW) were normalized by body weight (BW) and by tibial length (TL). Data (HW/TL, and HW/BW) were analyzed using standard software. As shown in FIG. 15, isoproterenol administration caused a significant increase in cardiac hypertrophy, as measured via normalized heart weight (HW/BW), relative to vehicle treated mice. Coadministration of SEQ ID NO: 87 with isoproterenol significantly attenuated the isoproterenol-induced cardiac hypertrophy.

Collagen content in each heart ventricle was measured via a hydroxyproline assay kit. The snap frozen whole heart ventricle tissues were weighed, chopped, and transferred into screw-capped tubes. Tissue was hydrolysed at 100 mg/ml in 6M HCl and incubated for 20 hrs at 95° C. in a calibrated oven or thermoblock. The hydrolyzed samples were then diluted with 4M HCl before assay analysis. To perform the assay analysis, 35 μl hydrolysate or hydroxyproline standard were mixed with 75 μl assay buffer in each well of a 96-well plate. The plate was incubated 20 minutes at room temperature while shaking. After, 75 μl of detection reagent was added to each well, the plate was mixed well and incubated 60 minutes at 60° C. in an oven or incubator. The plate was cooled down to room temperature, read at 570 nm, and hydroxyproline concentrations of each sample were determined by the standard curve. Data were analyzed using standard software. As shown in FIG. 16, isoproterenol administration caused a significant increase in fibrosis, as measured via collagen content, relative to vehicle treated mice. Coadministration of SEQ ID NO: 87 with isoproterenol significantly attenuated the isoproterenol-induced fibrosis.

### Example 10. Evaluating Safety, Tolerability, Pharmacokinetics, and Pharmacodynamics of a Relaxin-2 Fusion Protein Analog

This Example describes a double-blind, randomized, placebo-controlled, single ascending dose study being performed to assess the safety, tolerability, pharmacokinetics, pharmacodynamics, and immunogenicity of SEQ ID NO: 87 in healthy subjects.

### Study Design

Approximately 48 participants are enrolled in the study. A single ascending dose of SEQ ID NO: 87 or matching placebo (i.e., identical buffer without addition of SEQ ID NO: 87) was administered to study participants via intravenous infusion (IV) or subcutaneous (SC) injection in the doses described below. In each cohort, 6 participants receive SEQ ID NO: 87 and 2 participants receive placebo. Infusions were between 30 and 60 minutes in duration. Cohort A receives 0.3 mg/kg IV of SEQ ID NO: 87 or matching placebo. Cohort B receives 1 mg/kg IV of SEQ ID NO: 87 or matching placebo. Cohort C receives 300 mg SC of SEQ ID NO: 87 or matching placebo. In some cases, Cohort C is dosed concurrently with Cohort B. Cohort D receives 3 mg/kg IV of SEQ ID NO: 87 or matching placebo. Cohort E receives 600 mg SC of SEQ ID NO: 87 or matching placebo. In some cases, Cohort E is dosed concurrently with Cohort D. Cohort F receives 10 mg/kg IV of SEQ ID NO: 87 or matching placebo.

A master randomization schedule and code break envelopes was created, and delivered to the site's unblinded pharmacy before dosing. The active and placebo products are infused through a line that is covered to conceal active from placebo. A printed randomization schedule is generated using a permuted blocked fixed method. The people receiving the treatment(s), the people administering the treatment(s), and the people analyzing the results and data are blinded.

### Primary Outcome

The primary outcome of the study is to evaluate the safety and tolerability of SEQ ID NO: 87 after single ascending doses. The primary outcome is assessed by monitoring: 1) the incidence of adverse events (AEs) and serious adverse events (SAEs); 2) changes in clinical laboratory safety parameters including blood test results for hematology, serum chemistry, and coagulation studies; 3) changes in vital signs measurements; and 4) changes in electrocardiogram (ECG) findings. Adverse events are assessed by clinical examination, review of patient data and self-reporting. Adverse events are collected via verbal interview by clinic staff during the study period either in person during confinement or clinic visits or via telephone during remote contacts. Vital signs measurements includes resting heart rate and systolic and diastolic blood pressure (BP) using standard manual or electronic clinical procedures. These procedures are completed using standard nursing practices or, in the case of ECG, the instructions provided by the manufacturer. These individual data are utilized to determine safety by physician assessment. Additionally, the totality of the data presented are also factored into the determination of safety.

Adverse events and serious adverse events are assessed at Screening, Day-1, Day 1, Day 2, Day 3, Day 6, Day 8, Day 15, Day 29, Day 43, and Day 57 post dose administration. Laboratory safety parameters are assessed at Screening, Day-1, Day 1, Day 2, Day 8, Day 15, Day 29, Day 43, and Day 57 post dose administration. Vital signs are assessed at Screening, Day-1, Day 1, Day 2, Day 3, Day 6, Day 8, Day 15, Day 29, Day 43, and Day 57 post dose administration. ECG is performed at Screening, Day-1, Day 1, and Day 2 post dose administration.

### Secondary Outcomes

A secondary outcome of the study is to characterize the pharmacokinetic (PK) profile of SEQ ID NO: 87 in healthy participants after single ascending doses of SEQ ID NO: 87. Standard PK parameters are assessed: Cₘₐₓ, Tₘₐₓ, AUC-last (AUC from 0 to the last measurable concentration), AUC-inf (AUC from 0 to infinity), t_{1/2}, CL (clearance), and Vz (terminal phase volume of distribution). Blood sampling for PK parameter assessment occurs at Predose, End of infusion, 6 hrs, and 12 hrs post dose on Day 1, 24 hrs post dose on Day 2, 48 hours post dose on Day 3, 120 hours post dose on Day 6, 168 hours post dose on Day 8, Day 15, Day 29, Day 43 and Day 57 post dose.

Another secondary outcome of the study is to evaluate the pharmacodynamic (PD) effect of single ascending doses of SEQ ID NO: 87 in healthy participants. The change from baseline to day 2 is assessed in the following PD parameters: renal plasma flow (RPF), as measured by change in plasma para-aminohippurate (PAH) over time; renal blood flow (RBF); and filtration fraction (FF), as calculated by glomerular filtration rate (GFR) divided by RPF. Blood sampling for RPF/GFR assessment occurs on Days 2, 8, and 15 for IV Cohorts A, B and D and on Days 15 and 29 for IV Cohort F. Blood sampling for RPF/GFR assessment occurs on Days 2, 15, and 29 for SC Cohorts C and E.

### Subject Eligibility

Participants meet the following criteria to be enrolled in this study:
is a male or a female of non-childbearing potential between the ages of 18 and 55 years;
is judged to be in good health based upon medical history, physical examination, vital signs, ECGs, and routine laboratory tests;
has a body mass index (BMI) between 18 and 32 kg per meter square at screening;
understands the study procedures and agrees to participate in the study by giving written informed consent; and
is at least 18 years of age and no more than 55 years of age.

Participants were excluded from the study if any of the following criteria apply:
is mentally or legally incapacitated, has significant emotional problems at the time of the study, or has a history of significant psychiatric disorders at the discretion of the investigator;
has any clinically significant physical examination abnormalities observed during the screening visit or would not be a good candidate for participation in the study in the opinion of the investigator;
has clinically significant abnormal complete blood count, clinical chemistry, or urine analysis at screening or Day-1 (in asymptomatic participants, any abnormal laboratory results, including creatine phosphokinase within 3 times the upper limit of normal with suspected cause due to rigorous physical activities, may be repeated once during the screening period);
was hospitalized for any reason within 30 days of the screening visit;
has any history of clinically significant renal, neurologic, gastrointestinal, hepatic, or respiratory disease (note that subjects with fully resolved childhood asthma with no recurrence in adulthood may be enrolled);
has a history of anaphylaxis or other significant allergy in the opinion of the investigator;
has a history of clinically significant cardiovascular disease including arrhythmias, conduction abnormalities, or clinically significant abnormal vital signs;
was previously administered relaxin or relaxin fusion proteins; or
was dosed in any clinical research study evaluating another investigational drug (including biologics) or therapy (including specific immunotherapy) within 90 days or less than or equal to 5 half-lives (whichever is longer) of an investigational biologic drug, or less than or equal to 4 weeks for other investigational products, before the screening visit.

### Partial Results

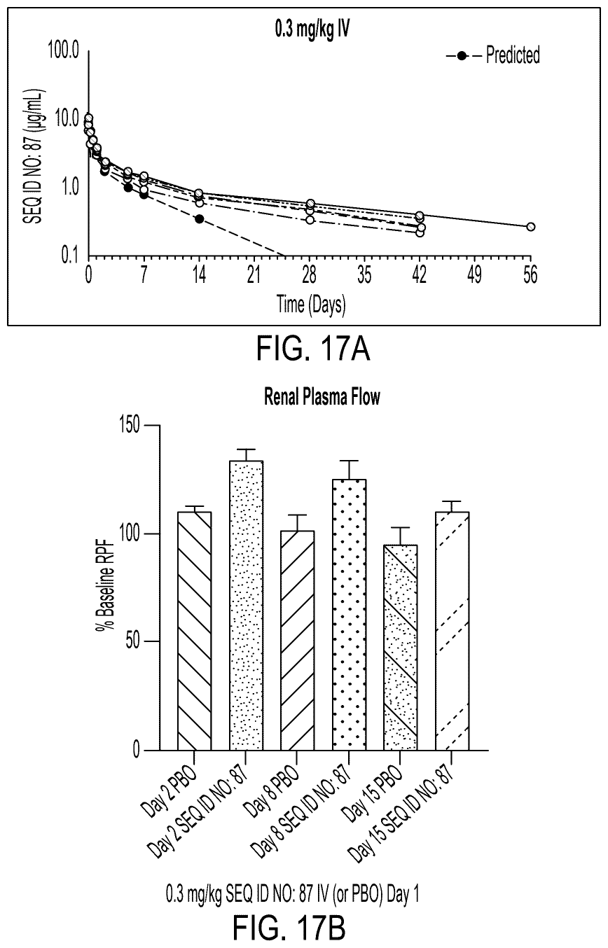

Preliminary PK and PD results were obtained from healthy human patients administered a single 0.3 mg/kg IV dose of SEQ ID NO: 87 (FIGS. 17A and 17B, respectively). As shown in FIG. 17A, SEQ ID NO: 87 demonstrated a desirable PK profile in four individual healthy patients after IV administration and a terminal half-life of 17 days, which is more than double the predicted terminal half-life of 6 days as obtained from simulations in non-human primate models (dashed line). The PD effect of SEQ ID NO: 87 in healthy human patients was investigated by assessing the change from baseline of renal plasma flow (RPF) at days 2, 8, and 15 (FIG. 17B). Briefly, patients were administered a bolus of para-aminohippurate (PAH) and plasma PAH was measured at days 2, 8, and 15. Effective RPF at each time point was calculated by using the formulas:

$$R P F ( ml / min ) = \frac{{Cl}_{P A H ml / min}}{extraction ratio} ,$$

$${Cl}_{P A H ml / min} = \frac{rate of P A H infusion}{{concentration}_{P A H mg / ml}} ,$$

As shown in FIG. 17B, a single 0.3 mg/kg IV dose of SEQ ID NO: 87 resulted in a substantial increase of at least about 30% in RPF on day 2 post dose compared to patients that received the placebo (PBO), and demonstrated persistence of effect for at least 8 days post dose.

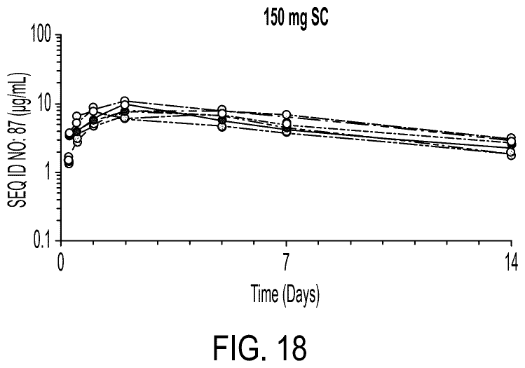

SEQ ID NO: 87 also demonstrated a desirable PK profile in healthy human patients administered a single 150 mg SC dose (FIG. 18), with terminal half-life not yet determined. Based on the data, SC bioavailability was in the range of 50-60%.

The preliminary data from healthy patients administered SEQ ID NO: 87 by IV and SC, supports a Q4W dosing schedule. The PK data showed low intersubject variability (≤20%) in serum concentrations, and no evidence of immune-mediated drug clearance. SEQ ID NO: 87 was found to be safe and well tolerated with minimal adverse events, and no drug-related SAEs. Expected on target effects were observed (e.g., mild orthostatic tachycardia without BP effect in some subjects), and no infusion or injection site reactions were reported.

### Example 11. Single-Dose, Open-Label Study to Evaluate the Safety, Tolerability, and Hemodynamic Effects of a Relaxin-2 Fusion Protein Analog

This prophetic Example describes a single-dose, open-label study to evaluate the safety, tolerability, and hemodynamics of SEQ ID NO: 87 in subjects with combined postcapillary and precapillary pulmonary hypertension (CpcPH) or isolated postcapillary pulmonary hypertension (IpcPH) and heart failure with preserved ejection fraction (HFpEF).

### Background and Rationale

SEQ ID NO: 87 is a relaxin peptide Fc-fusion biologic that is being developed for the treatment of combined precapillary and postcapillary pulmonary hypertension (CpcPH) and isolated postcapillary pulmonary hypertension (IpcPH). Relaxin is an insulin-like peptide, originally identified as a pregnancy-associated hormone, that has antifibrotic, anti-inflammatory, and vasodilatory properties in both men and women. It produces these effects by binding and activating its G-protein coupled receptor, relaxin family peptide receptor 1 (RXFP1), which then triggers a variety of signaling pathways including cAMP (cyclical adenosine monophosphate), cGMP (cyclical guanosine monophosphate), and MAPKs (mitogen activating protein kinases) as well as altering gene expression of transforming growth factor-beta (TGF-β), MMPs (matrix metalloproteinases), angiogenic growth factors, and endothelin-1 receptors.

Pulmonary hypertension (PH) is a condition that leads to dyspnea, reduced exercise capacity, and hypoxemia and has a high mortality risk. It is defined as a mean pulmonary arterial pressure (mPAP)≥20 mm Hg. Because this condition occurs in many different clinical settings, the World Health Organization (WHO) has divided PH into 5 different groups. CpcPH is a subgroup of WHO Group 2 pulmonary hypertension (PH), pH due to left-sided heart disease (PH-LHD). LHD includes the following: heart failure with preserved ejection fraction (EF), defined as heart failure with EF>50% (HFpEF); heart failure with reduced EF, defined as heart failure with EF<40% (HFrEF); and heart failure due to left heart valvular disease (either mitral or aortic valvular disease).

The unique hemodynamics seen in Group 2 PH-LHD that differentiates it from other forms of PH is an elevated pulmonary arterial wedge pressure (PAWP)≥15 mm Hg. The elevated wedge pressure reflects elevated left atrial pressure and is an indicator of abnormal left ventricular filling consistent with left heart disease. In group 2 PH, the elevation in mPAP may be due to primarily elevated post-capillary pressures due to elevations in PAWP. An additional subgroup of Group 2 PH has elevations in pre-capillary pressures, which are thought to be due to chronic elevations in mPAP leading to remodeling of the pulmonary arterial vasculature, similar to what is seen in pulmonary arterial hypertension (PAH; Group 1 PH). These 2 forms of PH-LHD are known as isolated post-capillary PH (IpcPH), and combined pre- and post-capillary PH (CpcPH).

CpcPH is defined hemodynamically by a pulmonary vascular resistance (PVR)≥3 Wood units, mean pulmonary artery pressure (mPAP)≥20 mm Hg, and pulmonary capillary wedge pressure (PCWP)≥15 mm Hg. In previous clinical trials, native human relaxin (serelaxin) was shown to modulate the hemodynamics of patients with acute heart failure associated with PH (Ponikowski et al. (2014) Eur. Heart J. 35 (7): 431-441). In one study, infusion of serelaxin for 20 hours resulted in a rapid and significant decline in mPAP, PVR, and PCWP. In a second trial, infusion of serelaxin for 24 hours in patients with stable heart failure and PH resulted in a sustained and significant increase in cardiac index (CI), and a sustained decrease in PCWP and systemic vascular resistance (SVR) [Group C in Dschietzig, et al. (2009) Ann. N Y Acad. Sci. 1160:387-392]. Clinical risk factors for CpcPH include obesity, hypertension, diabetes, and metabolic syndrome. Right ventricular (RV) dysfunction occurs more frequently in CpcPH than in IpcPH (Lteif, et al. (2021) J. Am. Heart Assoc. 10 (11): e020633).

IpcPH is a subtype of Group 2 PH (PH-LHD) where the elevated pulmonary artery pressure is due to passive backflow of elevated pressure from the left atrium and is not associated with major changes in the morphology of the pulmonary arteries and arterioles. It is defined hemodynamically by a PVR<3 Wood units, mPAP≥20 mm Hg, and PCWP≥15 mm Hg. Both IpcPH and CpcPH lead to an increase in right ventricular afterload, and this can lead to right heart dysfunction and death (Thenappan (2019) Advances in Pulmonary Hypertension. 18:87-91).

Patients with CpcPH have a worse prognosis than those with IpcPH. The 5-year survival rate for patients with CpcPH is 23% (Ibe et al. (2021) PLOS One. 16 (3): e0247987).

In this study, patients with CpcPH or IpcPH with HFpEF will be subjected to right heart catheterization (RHC) and will receive an intravenous (IV) dose of SEQ ID NO: 87. Hemodynamic measurements will be obtained for 8 hours after the dose of SEQ ID NO: 87 to determine the change in PVR and PCWP as well as other hemodynamic assessments over time.

### Study Design

Up to 25 participants may be enrolled in the study, across up to 3 cohorts, including a low-dose cohort (cohort A) with at least 2 participants with CpcPH, a cohort at the expected therapeutic dose (cohort B) with at least 6 participants with CpcPH and at least 3 participants with IpcPH, and a high dose cohort (cohort C) with at least 9 participants, including at least 6 participants with CpcPH and at least 3 participants with IpcPH.

During the screening period, participants will undergo assessments to ensure that they are appropriate for participation in the study.

Participants will require a 3-day admission to the clinical unit and then continue to be followed through Day 43. On Day-1, participants will be admitted to the clinical unit and have baseline assessments performed, including an echocardiogram. On Day 1, participants will have a right heart catheter (RHC) placed and baseline hemodynamic measurements will be obtained before dosing with SEQ ID NO: 87. Participants will be administered a dose of SEQ ID NO: 87 via at least a 30-minute intravenous (IV) infusion, and hemodynamic measures, heart rate (HR), and blood pressure (BP) will be obtained over an 8-hour period beginning after the end of the infusion. After the last hemodynamic measure is obtained, the RHC will be removed, and the participant will remain overnight for observation. Participants may be discharged from the clinical unit on Day 2 once all assessments are completed. The participant will return to the clinical unit on Days 8, 15, and 29 postdose for additional follow-up. Participants will return to the clinical unit for an end of study visit on Day 43.

### Treatment Plan

Each participant will receive a single open-label intravenous dose of SEQ ID NO: 87, based on a mg/kg body weight calculation (from Day-1), administered via at least a 30-minute infusion. SEQ ID NO: 87 will be administered with the participant in a semirecumbent position.

Up to 25 participants may be enrolled across up to 3 cohorts. Cohort A will enroll at least 2 participants with CpcPH at a proposed dose of 0.3 mg/kg SEQ ID NO: 87. Cohort B will enroll at least 6 participants with CpcPH and at least 3 participants with IpcPH at a dose of 1 mg/kg, and cohort C will enroll at least 6 participants with CpcPH and at least 3 participants with IpcPH at a dose of 3 mg/kg.

Because this is the initial clinical assessment of SEQ ID NO: 87 in patients with CpcPH or IpcPH, and the safety, PK, PD/efficacy, and immunogenicity profile of the compound are still being elucidated in healthy volunteers, this study is designed to have the flexibility to accommodate the inherent dynamic nature of Phase 1 clinical studies and the proposed doses may be adjusted. Participants will not receive a dose higher than 6 mg/kg.

Dosing of the first 2 participants in each cohort will be separated by at least 7 days. After the first 2 participants within a cohort are dosed, no stagger between dosing of participants within a cohort is required.

### Study Entry Criteria

Patients who meet the following inclusion criteria will be enrolled in the study:

1. Is a male or a female of non-childbearing potential between the ages of 18 and 80 years. A female of non-childbearing potential is a woman who meets either of the following criteria:
a. Is in a postmenopausal state defined as no menses for 12 months without an alternative medical cause and follicle-stimulating hormone (FSH) level consistent with post menopause (≥40 mIU/mL);
b. Has a documented hysterectomy, bilateral oophorectomy, or salpingectomy.
   2. Has one of the following:
a. Diagnosis of HFmrEF (heart failure with mid-range ejection fraction): defined as signs and symptoms of New York Heart Association (NYHA) class II-III heart failure and left ventricular ejection fraction (LVEF) 40% to 49%;
b. Diagnosis of HFpEF, defined as signs and symptoms of NYHA class II-III heart failure and LVEF≥50% and at least ONE of the following: i. Heart Failure Association-Pre-test assessment, Echocardiography and natriuretic peptide score, Functional testing in cases of uncertainty, Final etiology (HFA-PEFF) score ≥5 points; ii. HFA-PEFF score 2-4 points AND abnormal diastolic stress testing or invasive hemodynamic measurements.
   3. Has demonstrated CpcPH or IpcPH, based on RHC performed within 6 months of or during screening documenting the following:
a. For patients with CpcPH: PVR≥3 Wood units, or For patients with IpcPH: PVR<3 Wood units;
b. mPAP of >20 mm Hg;
c. PCWP>15 mm Hg or PCWP>12 mm Hg and ≤14 mm Hg with evidence on left atrial volume index (LAVI) on echocardiography of ≥34 mL/m².
   4. Is receiving chronic medication for heart failure or for any underlying condition, administered at a stable (per investigator) dose for ≥30 days before screening. Diuretics and/or anticoagulants are excepted from this rule but should not be newly started or stopped within 30 days of screening, and a prescribed dose change should not occur within 7 days of screening.
   5. Is able to adhere to the study visit schedule and understand and comply with all protocol requirements.
   6. Agrees not to participate in any other trials of investigational drugs/devices while enrolled in this study.
   7. Understands the study procedures and agrees to participate in the study by giving written informed consent

Patients are excluded from the study if any of the following exclusion criteria apply:

8. Has a confirmed diagnosis of PH in WHO Group 1, WHO Group 3, WHO Group 4, or WHO Group 5.
9. Has documented significant lung disease, defined as any of the following:
a. Chronic obstructive pulmonary disease (COPD) with post-bronchodilator forced expiratory volume in the first second (FEV1)<50% predicted;
b. Restrictive lung disease with total lung capacity <60% predicted;
c. More than mild interstitial lung disease (ILD), with forced vital capacity <70% or FEV1 <60% predicted (still appropriate if absence of more than mild ILD, fibrosis, or COPD on computed tomography imaging).
   10. Has cardiovascular comorbidities, which include any of the following:
a. Moderate to severe rheumatic heart disease with mitral stenosis;
b. Any history of greater than moderate mitral or aortic regurgitation valvular disease or greater than mild aortic or mitral stenosis. Severe tricuspid regurgitation may be included unless it is due to primary valvular disease, e.g., from endocarditis, carcinoid, or mechanical destruction or pacemaker lead;
c. Acute coronary syndrome, coronary artery bypass graft, or percutaneous coronary intervention within 3 months of screening;
d. Uncontrolled heart rate (HR) (>110 beats per minute [bpm] at screening) from atrial fibrillation or atrial flutter;
e. History of or anticipated heart transplant or ventricular assist device implantation;
f. Anticipated implantation of pacemaker, pacemaker implantation within 14 days of screening, or history of implantable cardioverter defibrillator placement;
g. Occurrence of myocardial infarction within 3 months of screening;
h. History of known pericardial constriction, hypertrophic cardiomyopathy, cardiac sarcoidosis, or amyloid cardiomyopathy and/or infectious or infiltrative myopathy;
i. Uncontrolled systemic hypertension as evidenced by sitting sBP>160 mm Hg or sitting diastolic blood pressure (dBP)>110 mm Hg during screening after a period of rest;
j. Sitting sBP<120 mm Hg or sitting dBP<50 mm Hg during screening;
k. Resting HR of <50 bpm or >115 bpm at screening;
l. Stroke within 90 days of screening;
m. Acutely decompensated heart failure that required hospitalization within 14 days of screening;
n. ECG during screening with heart rate-corrected QT interval (QTc) by Fridericia's method (QTcF)>470 milliseconds (ms) for males or >480 ms for females, or >500 ms if a ventricular conduction defect (right bundle branch block, left bundle branch block, or interventricular conduction delay) is present;
o. Personal or family history of Brugada syndrome;
p. Personal or family history of long QT syndrome unless the participant's ECG shows a normal QTc;
q. Arrhythmogenic right ventricular cardiomyopathy unless the participant has recent cardiac magnetic resonance imaging that shows no evidence of this diagnosis;
r. Any history of mitral or aortic valve replacement or repair (mechanical or biomechanical);
s. Complex congenital heart disease.
   11. Was hospitalized for any indication with discharge within 14 days of screening.
   12. Received any of the following classes of pulmonary arterial hypertension-specific therapies: endothelin receptor antagonists, prostacyclin analogs, soluble guanylate cyclase stimulators, and PDE5 (phosphodiesterase type 5) inhibitors within 30 days of screening.
   13. Received IV inotropes (e.g., dobutamine, dopamine, norepinephrine, vasopressin) within 30 days of screening.
   14. Has a known history of chronic liver disease, active hepatitis B infection (positive hepatitis B surface antigen and positive hepatitis B core antibodies), or hepatitis C infection (positive hepatitis C antibodies) at the screening visit. In patients with isolated positive hepatitis B core antibodies, a hepatitis B DNA test will be done to confirm the active hepatitis B. In patients with positive hepatitis C antibodies, hepatitis C RNA will be done to confirm the active hepatitis C.
   15. Has HIV infection or HIV seropositivity at the screening visit.
   16. Has active tuberculosis, as determined by Quantiferon Gold test and confirmed by chest x-ray.
   17. Has any of the following clinical laboratory values before screening as specified:
a. Serum alanine aminotransferase or aspartate aminotransferase levels >3× the upper limit of normal (ULN) or total bilirubin >3×ULN within 30 days of screening;
b. Estimated glomerular filtration rate <15 mL/min/1.73 m² (CKD EPI [Chronic Kidney Disease Epidemiology Collaboration] equation) within 30 days of screening or required renal replacement therapy within 90 days of screening;
c. Glycosylated hemoglobin (HbAlc)>9% within 30 days of screening;
d. Platelet count <50,000/mm³ within 30 days of screening.
   18. Has a history of severe allergic or anaphylactic reaction or hypersensitivity to recombinant proteins or excipients in the investigational product.
   19. Had major surgery within 60 days of screening. Participants must have completely recovered from any previous surgery before screening.
   20. Had a prior heart-lung transplant or has a life expectancy of <12 months.
   21. Is pregnant or breastfeeding.
   22. Has a history of active malignancy except for fully excised or treated basal cell carcinoma, cervical carcinoma in situ, or ≤2 squamous cell carcinomas of the skin.
   23. Has a history of clinically significant (as determined by the investigator) endocrine, hematologic, hepatic, (auto) immune, infectious (requiring chronic antibiotics), metabolic, urologic, pulmonary, neurologic, neuromuscular, dermatologic, psychiatric, renal, and/or another disease that may limit participation in the study.
   24. Has body mass index (BMI)<18 kg/m² or ≥40 kg/m².
   25. Has a history of drug or alcohol abuse or has a positive urine drug screen for prohibited drugs.
   26. Received any vaccination, including COVID-19 vaccination, within 2 weeks before dosing or plans to receive a vaccine during the study.
   27. Was previously administered relaxin or relaxin fusion proteins.
   28. Was dosed in any clinical research study evaluating another investigational drug (including biologics) or therapy (including specific immunotherapy) within 90 days or ≤5 half-lives (whichever is longer) of an investigational biologic drug, or ≤4 weeks for other investigational products, before the screening visit.
   29. Is mentally or legally incapacitated, has significant emotional problems at the time of the study, or has a history of significant psychiatric disorders at the discretion of the investigator.
   30. Has a history of any illness that, in the opinion of the investigator, might confound the results of the study or pose additional risk to the participant.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1-127. (canceled)

128. A polynucleotide comprising a nucleotide sequence encoding a fusion protein comprising, from N-terminus to C-terminus,
a first peptide;
a linker peptide; and
a second peptide,
wherein:
the first peptide comprises an amino acid sequence that has 0, 1, 2, 3, 4, or 5 amino acid modifications relative to the amino acid sequence of SEQ ID NO: 502 and the second peptide comprises an amino acid sequence that that has 0, 1, 2, 3, 4, or 5 amino acid modifications relative to the amino acid sequence of SEQ ID NO: 503 or 504; or
the first peptide comprises an amino acid sequence that has 0, 1, 2, 3, 4, or 5 amino acid modifications relative to the amino acid sequence of SEQ ID NO: 503 or 504 and the second peptide comprises an amino acid sequence that that has 0, 1, 2, 3, 4, or 5 amino acid modifications relative to the amino acid sequence of SEQ ID NO: 502.

129. The polynucleotide of claim 128, wherein:
(a) the first peptide comprises an amino acid sequence that has 0, 1, 2, 3, 4, or 5 amino acid modifications relative to the amino acid sequence of SEQ ID NO: 1, wherein the amino acid at at least one of positions 4 or 25 of the first peptide is not M; and the second peptide comprises an amino acid sequence that has 0, 1, 2, 3, 4, or 5 amino acid modifications relative to the amino acid sequence of SEQ ID NO: 8, wherein the amino acid at position 22 of the second peptide is not R; or
the first peptide comprises an amino acid sequence that has 0, 1, 2, 3, 4, or 5 amino acid modifications relative to the amino acid sequence of SEQ ID NO: 8, wherein the amino acid at position 22 of the second peptide is not R; and the second peptide comprises an amino acid sequence that has 0, 1, 2, 3, 4, or 5 amino acid modifications relative to the amino acid sequence of SEQ ID NO: 1, wherein the amino acid at at least one of positions 4 or 25 of the first peptide is not M.

130. The polynucleotide of claim 128, wherein the first peptide comprises the amino acid sequence set forth in SEQ ID NO: 1.

131. The polynucleotide of claim 128, wherein the amino acid sequence of the first peptide is 27, 28, or 29 amino acids in length, and/or consists of the amino acid sequence set forth in SEQ ID NO: 1.

132. The polynucleotide of claim 128, wherein:
the linker peptide comprises the amino acid sequence ASDAAGAX₈AX₉AGA (SEQ ID NO: 17), wherein:
X₈ is D, E, N, or Q; and
X₉ is D, E, N, or Q; or
the linker peptide comprises the amino acid sequence GGEGSGGEGX₁₀GGG (SEQ ID NO: 25), wherein:
X₁₀ is E or S.

133. The polynucleotide of claim 132, wherein X₈ is D, E, N, or Q, and X₉ is D, E, or Q; or X₈ is D, E, or Q, and X₉ is D, E, N, or Q.

134. The polynucleotide of claim 128, wherein the first linker peptide comprises the amino acid sequence set forth in SEQ ID NO: 21.

135. The polynucleotide of claim 128, wherein the amino acid sequence of the first linker peptide is 13, 14, or 15 amino acids in length, and/or consists of the amino acid sequence set forth in SEQ ID NO: 21.

136. The polynucleotide of claim 128, wherein the second peptide comprises the amino acid sequence set forth in SEQ ID NO: 8.

137. The polynucleotide of claim 128, wherein the amino acid sequence of the second peptide is 24 or 25 amino acids in length, and/or consists of the amino acid sequence set forth in SEQ ID NO: 8.

138. The polynucleotide of claim 128, wherein the amino acid sequence of the second peptide consists of the amino acid sequence set forth in SEQ ID NO: 260.

139. The polynucleotide of claim 128, wherein the fusion protein comprises or consists of the amino acid sequence set forth in SEQ ID NO: 31.

140. The polynucleotide of claim 128, further comprising an IgG Fc polypeptide, optionally wherein the IgG Fc polypeptide comprises:
the amino acid sequence of a human IgG1 Fc;
alanine at EU position 329;
alanine at each of EU positions 234 and 235;
alanine at each of EU positions 234, 235, and 329;
leucine and serine at EU positions 428 and 434, respectively; or
alanine, alanine, alanine, leucine, and serine at EU positions 234, 235, 329, 428, and 434, respectively.

141. The polynucleotide of claim 128, wherein the IgG Fc polypeptide comprises the amino acid sequence set forth in SEQ ID NO: 79 or 83.

142. The polynucleotide of claim 128, wherein the IgG Fc polypeptide is linked to the N-terminus of the first peptide, optionally via a second linker peptide, wherein the second linker peptide comprises the amino acid sequence GGS or EGGS (SEQ ID NO: 299).

143. The polynucleotide of claim 128, wherein the fusion protein comprises or consists of the amino acid sequence set forth in SEQ ID NO: 87.

144. An expression vector comprising the polynucleotide of claim 128.

145. A host cell comprising the polynucleotide of claim 128.

146. A method of producing the fusion protein encoded by the polynucleotide of claim 128, comprising culturing a host cell comprising the polynucleotide under conditions such that the fusion protein is produced.

147. A method of enhancing a relaxin-2-related activity in a cell or of enhancing activation of RXFP1 on a cell, comprising contacting the cell with a fusion protein comprising, from N-terminus to C-terminus,
a first peptide;
a linker peptide; and
a second peptide,
wherein:
the first peptide comprises an amino acid sequence that has 0, 1, 2, 3, 4, or 5 amino acid modifications relative to the amino acid sequence of SEQ ID NO: 502 and the second peptide comprises an amino acid sequence that that has 0, 1, 2, 3, 4, or 5 amino acid modifications relative to the amino acid sequence of SEQ ID NO: 503 or 504; or
the first peptide comprises an amino acid sequence that has 0, 1, 2, 3, 4, or 5 amino acid modifications relative to the amino acid sequence of SEQ ID NO: 503 or 504 and the second peptide comprises an amino acid sequence that that has 0, 1, 2, 3, 4, or 5 amino acid modifications relative to the amino acid sequence of SEQ ID NO: 502.

148. A method of treating a relaxin-associated disorder in a subject in need thereof, comprising administering to the subject an effective amount of:
(i) a fusion protein comprising, from N-terminus to C-terminus,
a first peptide;
a linker peptide; and
a second peptide,
wherein:
the first peptide comprises an amino acid sequence that has 0, 1, 2, 3, 4, or 5 amino acid modifications relative to the amino acid sequence of SEQ ID NO: 502 and the second peptide comprises an amino acid sequence that that has 0, 1, 2, 3, 4, or 5 amino acid modifications relative to the amino acid sequence of SEQ ID NO: 503 or 504; or
the first peptide comprises an amino acid sequence that has 0, 1, 2, 3, 4, or 5 amino acid modifications relative to the amino acid sequence of SEQ ID NO: 503 or 504 and the second peptide comprises an amino acid sequence that that has 0, 1, 2, 3, 4, or 5 amino acid modifications relative to the amino acid sequence of SEQ ID NO: 502;
(ii) a polynucleotide comprising a nucleotide sequence encoding the fusion protein;
(iii) an expression vector comprising the polynucleotide; or
(iv) a pharmaceutical composition comprising an effective amount of the fusion protein, the polynucleotide, or the expression vector.
